(19) United States
(12) Patent Application Publication
(10) Pub. No.: US 2026/0297575 A1
(43) Pub. Date: Oct. 1, 2026

(54) TREM COMPOSITIONS AND METHODS OF USE

(71) Applicant: Flagship Pioneering Innovations VI, LLC, Cambridge, MA (US)

(72) Inventors: Theonie Anastassiadis, Somerville, MA (US); William F. Kiesman, Wayland, MA (US); Hongchuan Yu, Chestnut Hill, MA (US); Stephen William Eichhorn, Medfork, MA (US); Christopher E. Arcadia, Barrington, RI (US); Daniel Abraham Glazier, Stoneham, MA (US); Christina Chung, Somerville, MA (US); Komal Sethia, Allston, MA (US)

(21) Appl. No.: 19/474,577

(22) PCT Filed: Apr. 12, 2024
(86) PCT No.: PCT/US2024/024492

§ 371 Date: Oct. 10, 2025

Related U.S. Application Data
(60) Provisional application No. 63/458,891, filed on Apr. 12, 2023.
(60) Provisional application No. 63/458,894, filed on Apr. 12, 2023.
(60) Provisional application No. 63/458,911, filed on Apr. 12, 2023.

(51) Int. Cl. C12N 15/113; A61K 9/51; C12N 15/88

(52) U.S. Cl. CPC C12N 15/113; A61K 9/5123; C12N 15/88; C12N 2310/315; C12N 2310/321; C12N 2310/322; C12N 2310/333; C12N 2310/334; C12N 2310/335; C12N 2310/336

(57) ABSTRACT

The invention relates generally to tRNA-based effector molecules having a non-naturally occurring modification and methods relating thereto.

### CLAIM OF PRIORITY

This application claims priority to U.S. Provisional Application No. 63/458,891, filed on Apr. 12, 2023; U.S. Provisional Application No. 63/458,894, filed on Apr. 12, 2023; and U.S. Provisional Application No. 63/458,911, filed on Apr. 12, 2023. The entire contents of each of the foregoing applications are incorporated herein by reference in their entirety.

### BACKGROUND

Transfer RNAs (tRNAs) are complex, naturally occurring RNA molecules that possess a number of functions including initiation and elongation of proteins.

### SUMMARY

The present disclosure features modified tRNA-based effector molecules (TREMs, e.g., a TREM or TREM fragment), as well as related compositions and uses thereof. TREMs are complex molecules which can mediate a variety of cellular processes. For example, the TREMs described herein may have the ability to: (i) support protein synthesis, (ii) be charged by a tRNA synthetase, (iii) be bound by an elongation factor, (iv) introduce an amino acid into a peptide chain, (v) support protein elongation, or (vi) support initiation of protein synthesis, e.g., in a cell.

Described herein are TREMs and related methods for optimizing the nucleotide sequence of a TREM, e.g., to improve a functional parameter of a TREM, e.g., to increase TREM stability. For example, the methods provided show that the nucleotide sequence of a TREM may be modified, e.g., by a substituting one nucleotide for another, to modulate a functional parameter of a TREM, for example to achieve an improvement in a functional parameter of a TREM. In an embodiment, the TREMs disclosed herein comprise at least one chemical modification (e.g., a non-naturally occurring modification), e.g., on a component nucleotide (e.g., a nucleobase or sugar) or within an internucleotide region, e.g., the TREM backbone. In another embodiment, the TREMs disclosed herein do not comprise a chemical modification (e.g., a non-naturally occurring modification), e.g., on a component nucleotide (e.g., a nucleobase or sugar) or within an internucleotide region, e.g., the TREM backbone). The present disclosure provides methods for tuning a functional parameter of a TREM by optimizing the nucleotide sequence, demonstrating a nucleotide sequence substitution (e.g., or further comprises a non-naturally occurring modification) may modulate a functional parameter of a TREM.

In one aspect, provided herein is a TREM comprising a sequence of Formula (A): [L1]x-[ASt Domain1]-[L2]x-[DH Domain]-[L3]x-[ACH Domain]-[VL Domain]-[TH Domain]-[L4]x-[ASt Domain2]-[L5]x, wherein independently, [L1] and [VL Domain], are optional; x, independently for every occurrence, is 0 or 1; and one of [L1], [ASt Domain1], [L2]-[DH Domain], [L3], [ACH Domain], [VL Domain], [TH Domain], [L4], and [ASt Domain2] comprises a nucleotide substitution relative to a reference sequence (e.g., a second TREM), wherein the TREM comprises a nucleotide substitution (e.g., a nucleotide mutation) in the TREM capable of modulating a functional parameter of the TREM. In an embodiment, a TREM described herein comprises a plurality of nucleotide substitutions (e.g., 1, 2, 3, 4, 5, 6, 7, 8, 9, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, or 80, e.g., relative to a reference sequence).

In another aspect, provided herein is a TREM comprising a sequence of Formula (A): [L1]x-[ASt Domain1]-[L2]x-[DH Domain]-[L3]x-[ACH Domain]-[VL Domain]-[TH Domain]-[L4]x-[ASt Domain2]-[L5]x, wherein independently, [L1] and [VL Domain], are optional; x, independently for every occurrence, is 0 or 1; and one of [L1], [ASt Domain1], [L2]-[DH Domain], [L3], [ACH Domain], [VL Domain], [TH Domain], [L4], and [ASt Domain2] comprises a nucleotide comprising a non-naturally occurring modification. In an embodiment, a TREM described herein comprises at least X contiguous nucleotides without a non-naturally occurring modification, wherein X is greater than 3, 4, 5, 6, 7, 8, 9, or 10; comprises at least 3, but less than all of the nucleotides of a type (e.g., A, T, C, G or U) comprise the same non-naturally occurring modification; comprises at least X nucleotides of a type (e.g., A, T, C, G or U) that do not comprise a non-naturally occurring modification, wherein X═ than 1, 2, 3, 4, 5, 6, 7, 8, 9, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, or 80; comprises no more than 1, 2, 3, 4, 5, 6, 7, 8, 9, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, or 80 nucleotides of a type (e.g., A, T, C, G or U) that comprise a non-naturally occurring modification; and/or comprises no more than 1, 2, 3, 4, 5, 6, 7, 8, 9, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, or 80 nucleotides of a type (e.g., A, T, C, G or U) that do not comprise a non-naturally occurring modification.

In an embodiment, the TREM Domain comprising the non-naturally occurring modification has a function, e.g., a domain function described herein.

In an aspect, provided herein is a TREM core fragment comprising a sequence of Formula B:

[L1]_{y}-[ASt Domain1]ₓ-[L2]_{y}-[DH Domain]_{y}-[L3]_{y}-[ACH Domain]ₓ-[VL Domain]_{y}-[TH Domain]_{y}-[L4]_{y}-[ASt Domain2]ₓ,
wherein x=1 and y=0 or 1; and one of [ASt Domain1], [ACH Domain], and [ASt Domain2] comprises a nucleotide having a non-naturally occurring modification.

In an embodiment, the TREM has the ability to support protein synthesis. In an embodiment, the TREM has the ability to be able to be charged by a synthetase. In an embodiment, the TREM has the ability to be bound by an elongation factor. In an embodiment, the TREM has the ability to introduce an amino acid into a peptide chain. In an embodiment, the TREM has the ability to support elongation. In an embodiment, the TREM has the ability to support initiation.

In an embodiment, the [ASt Domain 1] and/or [ASt Domain 2] comprising the non-naturally occurring modification has the ability to initiate or elongate a polypeptide chain.

In an embodiment, the [ACH Domain] comprising the non-naturally occurring modification has the ability to mediate pairing with a codon.

In an embodiment, y=1 for any one, two, three, four, five, six, all or a combination of [L1], [L2], [DH Domain], [L3], [VL Domain], [TH Domain], [L4].

In an embodiment, y=0 for any one, two, three, four, five, six, all or a combination of [L1], [L2], [DH Domain], [L3], [VL Domain], [TH Domain], [L4].

In an embodiment, y=1 for linker [L1], and L1 comprises a nucleotide having a non-naturally occurring modification.

In an embodiment, y=1 for linker [L2], and L2 comprises a nucleotide having a non-naturally occurring modification.

In an embodiment, y=1 for [DH Domain (DHD)], and DHD comprises a nucleotide having a non-naturally occurring modification. In an embodiment, the DHD comprising the non-naturally occurring modification has the ability to mediate recognition of aminoacyl-tRNA synthetase.

In an embodiment, y=1 for linker [L3], and L3 comprises a nucleotide having a non-naturally occurring modification.

In an embodiment, y=1 for [VL Domain (VLD)], and VLD comprises a nucleotide having a non-naturally occurring modification.

In an embodiment, y=1 for [TH Domain (THD)], and THD comprises a nucleotide having a non-naturally occurring modification. In an embodiment, the THD comprising the non-naturally occurring modification has the ability to mediate recognition of the ribosome.

In an embodiment, y=1 for linker [L4], and L4 comprises a nucleotide having a non-naturally occurring modification.

In another aspect, the disclosure provides a TREM fragment comprising a portion of a TREM, wherein the TREM comprises a sequence of Formula A: [L1]-[ASt Domain1]-[L2]-[DH Domain]-[L3]-[ACH Domain]-[VL Domain]-[TH Domain]-[L4]-[ASt Domain2], and wherein the TREM fragment comprises a non-naturally occurring modification.

In an embodiment, the TREM fragment comprises one, two, three or all or any combination of the following: (a) a TREM half (e.g., from a cleavage in the ACH Domain, e.g., in the anticodon sequence, e.g., a 5′half or a 3′ half); (b) a 5′ fragment (e.g., a fragment comprising the 5′ end, e.g., from a cleavage in a DH Domain or the ACH Domain); (c) a 3′ fragment (e.g., a fragment comprising the 3′ end, e.g., from a cleavage in the TH Domain); or (d) an internal fragment (e.g., from a cleavage in any one of the ACH Domain, DH Domain or TH Domain).

In an embodiment, the TREM fragment comprise (a) a TREM half which comprises a nucleotide having a non-naturally occurring modification.

In an embodiment, the TREM fragment comprise (b) a 5′ fragment which comprises a nucleotide having a non-naturally occurring modification.

In an embodiment, the TREM fragment comprise (c) a 3′ fragment which comprises a nucleotide having a non-naturally occurring modification.

In an embodiment, the TREM fragment comprise (d) an internal fragment which comprises a nucleotide having a non-naturally occurring modification.

In an embodiment of any of the TREMs, TREM core fragments, or TREM fragments disclosed herein, the TREM Domain comprises a plurality of nucleotides each having a non-naturally occurring modification. In an embodiment, the non-naturally occurring modification comprises a nucleobase modification, a sugar (e.g., ribose) modification, or a backbone modification. In an embodiment, the non-naturally occurring modification is a sugar (e.g., ribose) modification. In an embodiment, the non-naturally occurring modification is 2′-ribose modification, e.g., a 2′-OMe, 2′-halo (e.g., 2′-F), 2′-MOE, or 2′-deoxy modification. In an embodiment, the non-naturally occurring modification is a backbone modification, e.g., a phosphorothioate modification.

In an embodiment of any of the TREMs, TREM core fragments, or TREM fragments disclosed herein, the TREM sequence comprises a CCA sequence on a terminus, e.g., the 3′ terminus. In an embodiment, the TREM sequence does not comprise a CCA sequence on a terminus, e.g., the 3′ terminus.

In an embodiment of any of the TREMs, TREM core fragments, or TREM fragments disclosed herein, the non-naturally occurring modification is a modification in a base or a backbone of a nucleotide, e.g., a modification chosen from any one of Tables 5, 6, 7, 8 or or 9.

In an embodiment of any of the TREMs, TREM core fragments, or TREM fragments disclosed herein, the non-naturally occurring modification is a base modification chosen from a modification listed in Table 5.

In an embodiment of any of the TREMs, TREM core fragments, or TREM fragments disclosed herein, the TREM, TREM core fragment, or TREM fragment is encoded by a sequence provided in Table 1, e.g., any one of SEQ ID NOs 1-451.

In an embodiment of any of the TREMs, TREM core fragments, or TREM fragments disclosed herein, the TREM, TREM core fragment, or TREM fragment is encoded by a consensus sequence chosen from any one of SEQ ID NOs: 562-621.

In an embodiment of any of the TREMs, TREM core fragments, or TREM fragments disclosed herein, the TREM, TREM core fragment, or TREM fragment is encoded by a sequence provided in FIG. 3, e.g., any one of SEQ ID NOs: 625-1151. In an embodiment, the TREM, TREM core fragment, or TREM fragment comprises a TREM having at least 70%, 75%, 80%, 85%, 90%, 95%, or 99% sequence identity with a TREM provided in FIG. 3, e.g., any one of SEQ ID NOs: 625-1151. In an embodiment, the TREM, TREM core fragment, or TREM fragment comprises a sequence that differs by no more than 1, 2, 3, 4, 5, 6, 7, 8, 9, or 10 nucleotides from a TREM provided in FIG. 3, e.g., any one of SEQ ID NOs: 625-1151. In an embodiment, the TREM, TREM core fragment, or TREM fragment comprises 1, 2, 3, 4, 5, 6, 7, 8, 9, or 10 additional non-naturally occurring modifications compared with a TREM, TREM core fragment, or TREM fragment provided in FIG. 3, (e.g., 2′-ribose modifications or an internucleotide modification, e.g., 2′OMe, 2′-halo, 2′-MOE, 2′-deoxy, or phosphorothiorate modifications), e.g., any one of SEQ ID NOs: 625-1151.

In an embodiment of any of the TREMs, TREM core fragments, or TREM fragments disclosed herein, the TREM, TREM core fragment, or TREM fragment is a TREM provided in FIG. 3, e.g., any one of SEQ ID NOs: 625-1151. In an embodiment, the TREM, TREM core fragment, or TREM fragment comprises 1, 2, 3, 4, 5, 6, 7, 8, 9, or 10 additional non-naturally occurring modifications compared with a TREM provided in FIG. 3 (e.g., 2′-ribose modifications or an internucleotide modification, e.g., 2′OMe, 2′-halo, 2′-MOE, 2′-deoxy, or phosphorothiorate modifications), e.g., any one of SEQ ID NOs. 625-1151.

In another aspect, the disclosure provides a pharmaceutical composition comprising a TREM, a TREM core fragment, or a TREM fragment disclosed herein.

In another aspect, a TREM or a related composition thereof can be used, inter a/ia, to modulate a functional parameter (e.g., an expression parameter and/or a signaling parameter) of an RNA corresponding to, or a polypeptide encoded by, a nucleic acid sequence comprising an endogenous open reading frame (ORF) having a premature termination codon (PTC).

In another aspect, provided herein is a method of modulating a functional parameter of an mRNA corresponding to, or polypeptide encoded by, an endogenous open reading frame (ORF) in a subject, which ORF comprises a premature termination codon (PTC), contacting the subject with a TREM composition comprising a TREM, a TREM core fragment, or a TREM fragment disclosed herein in an amount and/or for a time sufficient to modulate the functional parameter of the mRNA or polypeptide, wherein the TREM, TREM core fragment or TREM fragment has an anticodon that pairs with the codon having the first sequence, thereby modulating the functional parameter in the subject. In an embodiment, the functional parameter comprises a signaling parameter and/or an expression parameter, e.g., as described herein.

In another aspect, disclosed herein is a method of modulating expression of a protein in a cell, wherein the protein is encoded by a nucleic acid comprising an endogenous open reading frame (ORF), which ORF comprises a premature termination codon (PTC), comprising contacting the cell with a TREM composition comprising a TREM, a TREM core fragment, or a TREM fragment disclosed herein in an amount and/or for a time sufficient to modulate expression of the encoded protein, wherein the TREM, TREM core fragment or TREM fragment has an anticodon that pairs with the PTC, thereby modulating expression of the protein in the cell. In an embodiment, the PTC comprises UAA, UGA or UAG.

In another aspect, provided herein is a method of increasing expression of a protein in a subject wherein the protein is encoded by a nucleic acid comprising an endogenous open reading frame (ORF), which ORF comprises a premature termination codon (PTC), comprising contacting the subject, in an amount and/or for a time sufficient to increase expression of the protein, with a TREM composition that (i) has an anticodon that pairs with the PTC, (ii) recognizes an aminoacyl-tRNA synthetase specific for Trp, Tyr, Cys, Glu, Lys, Gln, Ser, Leu, Arg, or Gly, (iii) comprises a sequence of Formula A, or (iv) comprises a non-naturally occurring modification. In an embodiment, the PTC comprises UAA, UGA or UAG. In an embodiment, the TREM composition comprises (i). In an embodiment, the TREM composition comprises (ii). In an embodiment, the TREM composition comprises (iii). In an embodiment, the TREM composition comprises (iv). In an embodiment, the TREM composition comprises two of (i)-(iv). In an embodiment, the TREM composition comprises three of (i)-(iv). In an embodiment, the TREM composition comprises each of (i)-(iv).

In another aspect, the disclosure provides a method of treating a subject having an endogenous open reading frame (ORF) which comprises a premature termination codon (PTC), comprising providing a TREM composition comprising a TREM, a TREM core fragment, or a TREM fragment disclosed herein, wherein the TREM comprises an anticodon that pairs with the PTC in the ORF; contacting the subject with the composition comprising a TREM, TREM core fragment or TREM fragment in an amount and/or for a time sufficient to treat the subject, thereby treating the subject. In an embodiment, the PTC comprises UAA, UGA or UAG.

In another aspect, the disclosure provides a method of treating a subject having an disease or disorder associated with a premature termination codon (PTC), comprising providing a TREM composition comprising a TREM, a TREM core fragment, or a TREM fragment disclosed herein; contacting the subject with the composition comprising a TREM, TREM core fragment or TREM fragment in an amount and/or for a time sufficient to treat the subject, thereby treating the subject. In an embodiment, the PTC comprises UAA, UGA or UAG. In an embodiment, the disease or disorder associated with a PTC is a disease or disorcer described herein, e.g., a cancer or a monogenic disease.

In an embodiment of any of the methods disclosed herein, the codon having the first sequence comprises a mutation (e.g., a point mutation, e.g., a nonsense mutation), resulting in a premature termination codon (PTC) chosen from UAA, UGA or UAG. In an embodiment, the codon having the first sequence or the PTC comprises a UAA mutation. In an embodiment, the codon having the first sequence or the PTC comprises a UGA mutation. In an embodiment, the codon having the first sequence or the PTC comprises a UAG mutation

In another aspect, the disclosure provides a method of making a TREM, a TREM core fragment, or a TREM fragment disclosed herein, comprising linking a first nucleotide to a second nucleotide to form the TREM.

In an embodiment, the TREM, TREM core fragment or TREM fragment is non-naturally occurring (e.g., synthetic).

In an embodiment, the TREM, TREM core fragment or TREM fragment is made by cell-free solid phase synthesis.

In another aspect, the disclosure provides a method of modulating a tRNA pool in a cell comprising: providing a TREM, a TREM core fragment, or a TREM fragment disclosed herein, and contacting the cell with the TREM, TREM core fragment or TREM fragment, thereby modulating the tRNA pool in the cell.

In an aspect, the disclosure provides a method of contacting a cell, tissue, or subject with a TREM, a TREM core fragment, or a TREM fragment disclosed herein, comprising: contacting the cell, tissue or subject with the TREM, TREM core fragment or TREM fragment, thereby contacting the cell, tissue, or subject with the TREM, TREM core fragment or TREM fragment.

In another aspect, the disclosure provides a method of delivering a TREM, TREM core fragment or TREM fragment to a cell, tissue, or subject, comprising: providing a cell, tissue, or subject, and contacting the cell, tissue, or subject, a TREM, a TREM core fragment, or a TREM fragment disclosed herein.

In an aspect, the disclosure provides a method of modulating a tRNA pool in a cell comprising an endogenous open reading frame (ORF), which ORF comprises a codon having a first sequence, comprising:
optionally, acquiring knowledge of the abundance of one or both of (i) and (ii), e.g., acquiring knowledge of the relative amounts of: (i) and (ii) in the cell, wherein (i) is a tRNA moiety having an anticodon that pairs with the codon of the ORF having a first sequence (the first tRNA moiety) and (ii) is an isoacceptor tRNA moiety having an anticodon that pairs with a codon other than the codon having the first sequence (the second tRNA moiety) in the cell;
contacting the cell with a TREM, a TREM core fragment, or a TREM fragment disclosed herein, wherein the TREM, TREM core fragment or TREM fragment has an anticodon that pairs with: the codon having the first sequence; or the codon other than the codon having the first sequence, in an amount and/or for a time sufficient to modulate the relative amounts of the first tRNA moiety and the second tRNA moiety in the cell,
thereby modulating the tRNA pool in the cell.

In another aspect, the disclosure provides a method of modulating a tRNA pool in a subject having an ORF, which ORF comprises a codon having a first sequence, comprising:
optionally, acquiring knowledge of the abundance of one or both of (i) and (ii), e.g., acquiring knowledge of the relative amounts of: (i) and (ii) in the subject, wherein (i) is a tRNA moiety having an anticodon that pairs with the codon of the ORF having a first sequence (the first tRNA moiety) and (ii) is an isoacceptor tRNA moiety having an anticodon that pairs with a codon other than the codon having the first sequence (the second tRNA moiety) in the subject;
contacting the subject with a TREM, a TREM core fragment, or a TREM fragment disclosed herein, wherein the TREM, TREM core fragment or TREM fragment has an anticodon that pairs with: the codon having the first sequence; or the codon other than the codon having the first sequence, in an amount and/or for a time sufficient to modulate the relative amounts of the first tRNA moiety and the second tRNA moiety in the subject,
thereby modulating the tRNA pool in the subject.

In an aspect, the disclosure provides a method of modulating a tRNA pool, e.g., a tRNA pool in a cell or subject having an endogenous ORF, comprising providing a composition comprising a TREM, a TREM core fragment, or a TREM fragment disclosed herein, wherein the TREM, TREM core fragment or TREM fragment comprises a nucleotide sequence modification and/or a non-naturally occurring modification; contacting the subject with the composition in an amount and/or for a time sufficient to modulate the tRNA pool in the subject, thereby modulating the tRNA pool in the subject.

In an aspect, the disclosure provides a method of modulating expression of a protein in a cell, wherein the protein is encoded by a nucleic acid comprising an ORF, which ORF comprises a codon having a mutation, comprising:
contacting the cell with a composition comprising a TREM, a TREM core fragment, or a TREM fragment disclosed herein in an amount and/or for a time sufficient to modulate expression of the encoded protein,
wherein the TREM, TREM core fragment or TREM fragment has an anticodon that pairs with the codon having the mutation,
thereby modulating expression of the protein in the cell.

In another aspect, the disclosure provides a method of modulating expression of a protein in a subject, wherein the protein is encoded by a nucleic acid comprising an endogenous ORF, which ORF comprises a codon having a mutation, comprising:
contacting the subject with a composition comprising a TREM, a TREM core fragment, or a TREM fragment disclosed herein, in an amount and/or for a time sufficient to modulate expression of the encoded protein,
wherein the TREM, TREM core fragment or TREM fragment has an anticodon that pairs with the codon having the mutation,
thereby modulating expression of the protein in the subject.

In an embodiment of any of the methods disclosed herein, the mutation in the ORF is a nonsense mutation, e.g., resulting in a premature stop codon chosen from UAA, UGA or UAG. In an embodiment, the stop codon is UAA. In an embodiment, the stop codon is UGA. In an embodiment, the stop codon is UAG.

In an embodiment of any of the methods disclosed herein, the TREM comprises an anticodon that pairs with a stop codon.

TREMs of the disclosure include TREMs, TREM core fragments and TREM fragments. TREMs, TREM core fragments or TREM fragments can be modified with non-naturally occurring modifications to, e.g., increase the level and/or activity (e.g., stability) of the TREM. Pharmaceutical TREM compositions, e.g., comprising TREMs having a non-naturally occurring modification, can be administered to cells, tissues or subjects to modulate these functions, e.g., in vitro or in vivo. Disclosed herein are TREMs, TREM core fragments or TREM fragments comprising non-naturally occurring modifications, TREM compositions, preparations, methods of making TREM compositions and preparations, and methods of using the same.

In an embodiment, the TREM, TREM core fragment, and TREM fragments comprise a non-naturally occurring modification that improves stability or enhances activity of the TREM, TREM core fragment, or TREM fragment.

Additional features of any of the aforesaid TREMs, TREM core fragments, TREM fragments, TREM compositions, preparations, methods of making TREM compositions and preparations, and methods of using TREM compositions and preparations include one or more of the features in the Enumerated Embodiments, Figures, Description, Examples, or Claims.

Those skilled in the art will recognize or be able to ascertain using no more than routine experimentation, many equivalents to the specific embodiments of the invention described herein. Such equivalents are intended to be encompassed by the following Enumerated Embodiments, Drawings, Description, Examples, or Claims.

### BRIEF DESCRIPTION OF DRAWINGS

FIGS. 1-2 are graphs showing activity of TREMs for read-though of a premature termination codon (PTC) engineered in a NanoLuc reporter protein to produce a functional NanoLuc protein in a cell line stably expressing the NanoLuc reporter protein upon transduction with TREM expression constructs. FIG. 1 is a graph comparing the log 2 PTC read-though activity of TREMs consisting of the nucleotide sequence scaffolds listed in Table 7 modified with each of the chemical modification patterns listed in Table 6. FIG. 2 is a graph comparing the log 2 PTC readthrough activity of TREMs consisting of the nucleotide sequence scaffolds listed in Table 7 comprising 0 to 41 chemically modified nucleotides.

FIG. 3 is a table listing exemplary TREMs described herein.

FIG. 4 shows the abundance of nucleotide substitutions at each position of various TREM scaffolds in a pool of hits resulting from a pooled screen for PTC read-through activity of TREMs comprising nucleotide substitutions. The SEQ ID NOs indicate reference TREMs from which the pool of TREMs having nucleotide substitutions is generated for screening. FIG. 4A is a schematic representation of a TREM indicating regions where nucleotide substitutions typically result in increased PTC read-through activity, e.g., typically positive, typically result in decreased PTC read-through activity, e.g., typically negative, or result in increased PTC read-through activity for certain scaffolds, e.g., scaffold-specific positive. FIG. 4B shows the normalized hit abundance for nucleotide substitutions at each position in various TREM scaffolds, e.g., the frequency at which each position within a TREM comprises a nucleotide substitution in the pool of hits from a screen for increased PTC read-through activity.

FIG. 5 is a heatmap showing the enrichment of nucleotide substitutions, e.g., mutated sites, at each position of various TREM scaffolds in a pool of hits resulting from a pooled screen for PTC read-through activity of TREMs comprising nucleotide substitutions, e.g., showing the frequency at which each position within a TREM comprises a nucleotide substitution in the pool of hits from a screen for increased PTC read-through activity. The SEQ ID NOs indicate reference TREMs from which the pool of TREMs comprising nucleotide substitutions is generated for screening. The boxed regions indicate positions where nucleotide substitutions result in increased activity of a TREM.

FIGS. 6-15 are graphs showing the abundance of nucleotide substitutions at each position of various TREM scaffolds in a pool of hits resulting from a pooled screen for PTC read-through activity of TREMs comprising nucleotide substitutions. The SEQ ID NOs indicate reference TREMs from which the pool of TREMs comprising nucleotide substitutions is generated for screening. FIG. 6 shows the normalized hit abundance for nucleotide substitutions at each position in an Arg-TGA reference scaffold (SEQ ID NO: 1638). FIG. 7 shows the normalized hit abundance for nucleotide substitutions at each position in a Gln-TAG reference scaffold (SEQ ID NO: 1835). FIG. 8 shows the normalized hit abundance for nucleotide substitutions at each position in two Gln-TAG reference scaffolds (SEQ ID NOs: 1660 and 1654) resulting from the first round of pooled screening shown in FIG. 7. FIG. 9 shows the aggregate normalized hit abundance for nucleotide substitutions at each position in three Glu-TAG reference scaffolds (SEQ ID NOs: 1867, 2000, and 2001). FIG. 10 shows the normalized hit abundance for nucleotide substitutions at each position in a Glu-TAG reference scaffold (SEQ ID NO: 1867). FIG. 11 shows the normalized hit abundance for nucleotide substitutions at each position in a Glu-TAG reference scaffold (SEQ ID NO: 2000). FIG. 12 shows the normalized hit abundance for nucleotide substitutions at each position in two Leu-TAG reference scaffolds (SEQ ID NOs: 2016 and 2017). FIG. 13 shows the normalized hit abundance for nucleotide substitutions at each position in two Tyr-TAG reference scaffolds (SEQ ID NOs: 2022 and 2023). FIG. 14 shows the normalized hit abundance for nucleotide substitutions at each position in two Ser-TAG reference scaffolds (SEQ ID NOs: 2020 and 2021). FIG. 15 shows the normalized hit abundance for nucleotide substitutions at each position in two Lys-TAG reference scaffolds (SEQ ID NOs: 2018 and 2019).

FIG. 16 is a heatmap showing enrichment for disrupted base-pairing at each position of various TREM scaffolds resulting from a pooled screen for PTC read-through activity of TREMs having nucleotide substitutions. The SEQ ID NOs indicate reference TREMs from which the pool of TREMs comprising nucleotide substitutions is generated for screening. The boxed regions indicate positions where nucleotide substitutions that disrupt base pairing result in increased activity of a TREM.

FIG. 17 is a scatter plot showing the enrichment score for TREMs in a Gln-TAG pooled screen. Candidates are colored in gray, negative control TREMs in black, and human-based tRNAs with anticodons cognate to the TAG PTC in as positive controls magenta. The hit threshold is indicated as vertical and horizontal dashed lines, with candidates in the upper right quadrant designated as hits.

FIG. 18 is a graph showing a luciferase reporter assay performed on candidates shown in FIG. 17 selected for hit validation. The starting sequence from which variants were then created is shown in magenta, and the activity of all tested sequences was evaluated at two concentrations.

FIGS. 19A-B show the enrichment score for TREMs in a Glu-TAG pooled screen. FIG. 19A shows candidate TREMs are colored in light gray, negative control TREMs in dark gray, and human-based tRNAs with anticodons cognate to the TAG PTC as positive controls in magenta. The hit threshold is indicated as vertical and horizontal dashed lines, with candidates in the upper right quadrant designated as hits. FIG. 19B shows Glu-TAG TREM hits resulting from the screen.

FIGS. 20A-B show a luciferase reporter assay performed on candidates shown in FIG. 19 selected for hit validation.

FIGS. 21A-C show schematics of TREMs from hits shown in FIG. 20. FIG. 21A shows the parent TREM. FIG. 21B shows a Glu-TAG TREM hit. FIG. 21C shows a Glu-TAG TREM hit.

FIG. 22 is a scatter plot showing the results of a luciferase reporter assay performed on candidate TREMs selected for hit validation as both synthesized oligonucleotides and as lentiviral particles. The parent sequence is shown in magenta, with hits shown in gray.

FIG. 23 is a table listing exemplary TREMs described herein.

FIG. 24 is a table listing the effect of nucleotide substitutions on the PTC read-through activity of TREMs listed in FIG. 23. “Sub.” indicates substitution; “compens.” indicates compensatory; “rel.” indicates relative.

FIG. 25 is a table comparing the PTC read-through activity of hits from a pooled screen of TREMs comprising nucleotide substitutions measured by validation as synthesized oligonucleotides or by lentiviral delivery.

### DETAILED DESCRIPTION OF CERTAIN EMBODIMENTS

The present disclosure features tRNA-based effector molecules (TREMs), compositions, and related methods useful for optimizing a functional parameter of the TREM, for example, by introducing a nucleotide sequence modification, a non-naturally occurring chemical modification, or both into the TREM sequence. As disclosed herein, TREMs are complex molecules which can mediate a variety of cellular processes. The inventors have discovered that the nucleotide sequence of a TREM can be optimized to modulate a functional parameter of a TREM; the methods described herein describe how a plurality of sequences (e.g., tens, hundreds, or thousands) may be screened in order to select for a TREM sequence bearing a different functional readout. For example, out of a pooled screen of TREM sequences in which each nucleotide position of the TREM has been substituted with another nucleotide sequence, TREM sequences may be analyzed to select those TREMs that contain certain nucleotide substitutions that may, e.g., enhance the TREM for a certain function, such as improved readthrough of a premature termination codon (PTC), in a transcript.

Pharmaceutical TREM compositions, e.g., the TREMs described herein, can be administered to a cell, a tissue, or to a subject to modulate certain cellular functions. Also disclosed herein are methods of modulating expression of a protein in a subject or cell, wherein the protein is encoded by a nucleic acid comprising an endogenous open reading frame (ORF) having a first sequence, e.g., a mutation, e.g., a premature termination codon (PTC), and methods of treating a subject having an endogenous open reading frame (ORF) which comprises a premature termination codon (PTC). Further disclosed herein are TREMs comprising a non-naturally occurring modification, methods of making the same and compositions thereof.

### Definitions

“Acquire” or “acquiring” as the terms are used herein, refer to obtaining possession of a value, e.g., a numerical value, by “directly acquiring” or “indirectly acquiring” the physical entity or value. “Directly acquiring” refers to performing a process (e.g., performing an analytical method) to obtain the value. “Indirectly acquiring” refers to receiving the value from another party or source (e.g., a third party laboratory that directly acquired the or value).

A “disease or disorder associated with a PTC” as that term is used herein includes, but is not limited to, a disease or disorder in which cells express, or at one time expressed, a polypeptide encoded by an ORF comprising a PTC. In some embodiments, a disease associated with a PTC is chosen from: a proliferative disorder (e.g., a cancer), a genetic disorder, a metabolic disorder, an immune disorder, an inflammatory disorder or a neurological disorder. Exemplary diseases or disorders associated with a PTC are provided in any one of Tables 15, 16 and 17. In an embodiment, the disease associated with a PTC is a cancer. In an embodiment, the disease associated with a PTC is a monogenic disease.

An “isoacceptor,” as that term is used herein, refers to a plurality of tRNA molecule or TREMs wherein each molecule of the plurality comprises a different naturally occurring anticodon sequence and each molecule of the plurality mediates the incorporation of the same amino acid and that amino acid is the amino acid that naturally corresponds to the anticodons of the plurality.

A “modification,” as that term is used herein in regard to a TREM, may refer to a sequence modification or a chemical modification of the TREM. As this term is used to in reference to a sequence modification, the modification may include a nucleotide addition, nucleotide deletion or nucleotide substitution. As this term is used in reference to a chemical modification, the modification may include a modification of the chemical structure, e.g., a covalent modification, of the subject nucleotide. The chemical modification can be naturally occurring or non-naturally occurring. In an embodiment, the modification is non-naturally occurring. In an embodiment, the modification is naturally occurring. In an embodiment, the modification is a synthetic modification. In an embodiment, the modification is a modification provided in Table 5.

A “naturally occurring nucleotide,” as that term is used herein, refers to a nucleotide that does not comprise a non-naturally occurring modification. In an embodiment, it includes a naturally occurring modification.

A “non-naturally occurring modification,” as that term is used herein with reference to a nucleotide, refers to a chemical modification that: (a) a cell, e.g., a human cell, does not make on an endogenous tRNA; or (b) a cell, e.g., a human cell, can make on an endogenous tRNA but wherein such modification is in a location in which it does not occur on a native tRNA, e.g., the modification is in a domain, linker or arm, or on a nucleotide and/or at a position within a domain, linker or arm, which does not have such modification in nature. In either case, the modification is added synthetically, e.g., in a cell free reaction, e.g., in a solid state or liquid phase synthetic reaction. In an embodiment, the non-naturally occurring modification is a modification that is not present (in identity, location or position) if a sequence of the TREM is expressed in a mammalian cell, e.g., a HEK293 cell line. Exemplary non-naturally occurring modifications are found in Table 5.

A “non-naturally modified nucleotide,” as that term is used herein, refers a nucleotide comprising a non-naturally occurring modification on or of a sugar, nucleobase, or phosphate moiety.

A “nucleotide,” as that term is used herein, refers to an entity comprising a sugar, typically a pentameric sugar; a nucleobase; and a phosphate linking group. In an embodiment, a nucleotide comprises a naturally occurring, e.g., naturally occurring in a human cell, nucleotide, e.g., an adenine, thymine, guanine, cytosine, or uracil nucleotide.

A “premature termination codon” or “PTC” as those terms are used herein, refer to a stop codon that occurs in an open reading frame (ORF) of a DNA or mRNA. In an embodiment, a PTC occurs at a position upstream of a naturally occurring stop codon in an ORF. In an embodiment, a PTC that occurs upstream of a naturally occurring stop codon, e.g., in an ORF, results in modulation of a production parameter of the corresponding mRNA or polypeptide encoded by the ORF. In an embodiment, a PTC can differ (or arise) from a pre-mutation sequence by a point mutation, e.g., a nonsense mutation. In an embodiment, a PTC can differ (or arise) from a pre-mutation sequence by a genetic change, e.g., abnormality, other than a point mutation, e.g., a frameshift, a deletion, an insertion, a rearrangement, an inversion, a translocation, a duplication, or a transversion. In an embodiment, a PTC results in the production of a truncated protein which lacks a native activity or which is associated with a mutant, disease, or other unwanted phenotype. In an embodiment, the ORF comprising the PTC is an ORF from a tumor suppressor gene. In an embodiment, the mutation giving rise to the PTC is a driver mutation, e.g., a mutation that provides a growth advantage to a tumor cell.

A “functional parameter,” refers to an expression parameter and/or a signaling parameter. In an embodiment a functional parameter is an expression parameter. An expression parameter includes an expression parameter of a polypeptide or protein encoded by the endogenous ORF having a first sequence or PTC; or an expression parameter of an RNA, e.g., messenger RNA, encoded by the endogenous ORF having a first sequence or PTC. In an embodiment, an expression parameter can include:
(a) protein translation;
(b) expression level (e.g., of polypeptide or protein, or mRNA);
(c) post-translational modification of polypeptide or protein;
(d) folding (e.g., of polypeptide or protein, or mRNA),
(e) structure (e.g., of polypeptide or protein, or mRNA),
(f) transduction (e.g., of polypeptide or protein),
(g) compartmentalization (e.g., of polypeptide or protein, or mRNA),
(h) incorporation (e.g., of polypeptide or protein, or mRNA) into a supermolecular structure, e.g., incorporation into a membrane, proteasome, or ribosome,
(i) incorporation into a multimeric polypeptide, e.g., a homo or heterodimer, and/or
(j) stability.

In an embodiment, a functional parameter is a signaling parameter. A signaling parameter can include:
(1) modulation of a signaling pathway, e.g., a cellular signaling pathway which is downstream or upstream of the protein encoded by the endogenous ORF having a first sequence or PTC;
(2) cell fate modulation;
(3) ribosome occupancy modulation;
(4) protein translation modulation;
(5) mRNA stability modulation;
(6) protein folding and structure modulation;
(7) protein transduction or compartmentalization modulation; and/or
(8) protein stability modulation.

An “ORF having a PTC” as that phrase is used herein, refers to an open reading frame (ORF) which comprises a premature termination codon (PTC). In an embodiment, the ORF having the PTC is associated with a disease or disorder associated with a PTC, e.g., as described herein, e.g., a disease or disorder listed in any one of Tables 15, 16 and 17. In an embodiment, the ORF having the PTC is not associated with a disease or disorder associated with a PTC.

A “stop codon” as that term is used herein, refers to a three nucleotide contiguous sequence within messenger RNA that specifies a termination of translation. For example, UAG, UAA, UGA (in RNA) and TAG, TAA or TGA (in DNA) are stop codons. The stop codons are also known as amber (UAG), ochre (UAA), and opal (UGA).

A “tRNA-based effector molecule” or “TREM,” as that term is used herein, refers to an RNA molecule comprising a structure or property from (a)-(v) below, and which is a recombinant TREM, a synthetic TREM, or a TREM expressed from a heterologous cell. The TREMs described in the present invention are synthetic molecules and are made, e.g., in a cell free reaction, e.g., in a solid state or liquid phase synthetic reaction. TREMs are chemically distinct, e.g., in terms of primary sequence, type or location of modifications from the endogenous tRNA molecules made in cells, e.g., in mammalian cells, e.g., in human cells. A TREM can have a plurality (e.g., 2, 3, 4, 5, 6, 7, 8, 9) of the structures and functions of (a)-(v).

In an embodiment, a TREM is non-native, as evaluated by structure or the way in which it was made.

In an embodiment, a TREM comprises one or more of the following structures or properties:
(a′) an optional linker region of a consensus sequence provided in the “Consensus Sequence” section, e.g., a Linker 1 region;
(a) an amino acid attachment domain that binds an amino acid, e.g., an acceptor stem domain (AStD), wherein an AStD comprises sufficient RNA sequence to mediate, e.g., when present in an otherwise wildtype tRNA, acceptance of an amino acid, e.g., its cognate amino acid or a non-cognate amino acid, and transfer of the amino acid (AA) in the initiation or elongation of a polypeptide chain. Typically, the AStD comprises a 3′-end adenosine (CCA) for acceptor stem charging which is part of synthetase recognition. In an embodiment the AStD has at least 75, 80, 85, 85, 90, 95, or 100% identity with a naturally occurring AStD, e.g., an AStD encoded by a nucleic acid in Table 1. In an embodiment, the TREM can comprise a fragment or analog of an AStD, e.g., an AStD encoded by a nucleic acid in Table 1, which fragment in embodiments has AStD activity and in other embodiments does not have AStD activity. (One of ordinary skill can determine the relevant corresponding sequence for any of the domains, stems, loops, or other sequence features mentioned herein from a sequence encoded by a nucleic acid in Table 1. E.g., one of ordinary skill can determine the sequence which corresponds to an AStD from a tRNA sequence encoded by a nucleic acid in Table 1.)

In an embodiment the AStD falls under the corresponding sequence of a consensus sequence provided in the “Consensus Sequence” section, or differs from the consensus sequence by no more than 1, 2, 5, or 10 positions;

In an embodiment, the AStD comprises residues R₁-R₂-R₃-R₄-R₅-R₆-R₇ and residues R₆₅-R₆₆-R₆₇-R₆₈-R₆₉-R₇₀-R₇₁ of Formula I _{ZZZ}, wherein ZZZ indicates any of the twenty amino acids;

In an embodiment, the AStD comprises residues R₁-R₂-R₃-R₄-R₅-R₆-R₇ and residues R₆₅-R₆₆-R₆₇-R₆₈-R₆₉-R₇₀-R₇₁ of Formula II _{ZZZ}, wherein ZZZ indicates any of the twenty amino acids;

In an embodiment, the AStD comprises residues R₁-R₂-R₃-R₄-R₅-R₆-R₇ and residues R₆₅-R₆₆-R₆₇-R₆₈-R₆₉-R₇₀-R₇₁ of Formula III _{ZZZ}, wherein ZZZ indicates any of the twenty amino acids;
(a′-1) a linker comprising residues R₈-R₉ of a consensus sequence provided in the “Consensus Sequence” section, e.g., a Linker 2 region;
(b) a dihydrouridine hairpin domain (DHD), wherein a DHD comprises sufficient RNA sequence to mediate, e.g., when present in an otherwise wildtype tRNA, recognition of aminoacyl-tRNA synthetase, e.g., acts as a recognition site for aminoacyl-tRNA synthetase for amino acid charging of the TREM. In embodiments, a DHD mediates the stabilization of the TREM's tertiary structure. In an embodiment the DHD has at least 75, 80, 85, 85, 90, 95, or 100% identity with a naturally occurring DHD, e.g., a DHD encoded by a nucleic acid in Table 1. In an embodiment, the TREM can comprise a fragment or analog of a DHD, e.g., a DHD encoded by a nucleic acid in Table 1, which fragment in embodiments has DHD activity and in other embodiments does not have DHD activity.

In an embodiment the DHD falls under the corresponding sequence of a consensus sequence provided in the “Consensus Sequence” section, or differs from the consensus sequence by no more than 1, 2, 5, or 10 positions;

In an embodiment, the DHD comprises residues R₁₀-R₁₁-R₁₂-R₁₃-R₁₄ R₁₅-R₁₆-R₁₇-R₁₈-R₁₉-R₂₀-R₂₁-R₂₂-R₂₃-R₂₄-R₂₅-R₂₆-R₂₇-R₂₈ of Formula I _{ZZZ}, wherein ZZZ indicates any of the twenty amino acids;

In an embodiment, the DHD comprises residues R₁₀-R₁₁-R₁₂-R₁₃-R₁₄ R₁₅-R₁₆-R₁₇-R₁₈-R₁₉-R₂₀-R₂₁-R₂₂-R₂₃-R₂₄-R₂₅-R₂₆-R₂₇-R₂₈ of Formula II _{ZZZ}, wherein ZZZ indicates any of the twenty amino acids;

In an embodiment, the DHD comprises residues R₁₀-R₁₁-R₁₂-R₁₃-R₁₄ R₁₅-R₁₆-R₁₇-R₁₈-R₁₉-R₂₀-R₂₁-R₂₂-R₂₃-R₂₄-R₂₅-R₂₆-R₂₇-R₂₈ of Formula III _{ZZZ}, wherein ZZZ indicates any of the twenty amino acids;
(b′-1) a linker comprising residue R₂₉ of a consensus sequence provided in the “Consensus Sequence” section, e.g., a Linker 3 region;
(c) an anticodon that binds a respective codon in an mRNA, e.g., an anticodon hairpin domain (ACHD), wherein an ACHD comprises sufficient sequence, e.g., an anticodon triplet, to mediate, e.g., when present in an otherwise wildtype tRNA, pairing (with or without wobble) with a codon; In an embodiment the ACHD has at least 75, 80, 85, 85, 90, 95, or 100% identity with a naturally occurring ACHD, e.g., an ACHD encoded by a nucleic acid in Table 1. In an embodiment, the TREM can comprise a fragment or analog of an ACHD, e.g., an ACHD encoded by a nucleic acid in Table 1, which fragment in embodiments has ACHD activity and in other embodiments does not have ACHD activity.

In an embodiment the ACHD falls under the corresponding sequence of a consensus sequence provided in the “Consensus Sequence” section, or differs from the consensus sequence by no more than 1, 2, 5, or 10 positions;

In an embodiment, the ACHD comprises residues —R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆ of Formula I _{ZZZ}, wherein ZZZ indicates any of the twenty amino acids;

In an embodiment, the ACHD comprises residues —R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆ of Formula II _{ZZZ}, wherein ZZZ indicates any of the twenty amino acids;

In an embodiment, the ACHD comprises residues —R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆ of Formula III _{ZZZ}, wherein ZZZ indicates any of the twenty amino acids;
(d) a variable loop domain (VLD), wherein a VLD comprises sufficient RNA sequence to mediate, e.g., when present in an otherwise wildtype tRNA, recognition of aminoacyl-tRNA synthetase, e.g., acts as a recognition site for aminoacyl-tRNA synthetase for amino acid charging of the TREM. In embodiments, a VLD mediates the stabilization of the TREM's tertiary structure. In an embodiment, a VLD modulates, e.g., increases, the specificity of the TREM, e.g., for its cognate amino acid, e.g., the VLD modulates the TREM's cognate adaptor function. In an embodiment the VLD has at least 75, 80, 85, 85, 90, 95, or 100% identity with a naturally occurring VLD, e.g., a VLD encoded by a nucleic acid in Table 1. In an embodiment, the TREM can comprise a fragment or analog of a VLD, e.g., a VLD encoded by a nucleic acid in Table 1, which fragment in embodiments has VLD activity and in other embodiments does not have VLD activity.

In an embodiment the VLD falls under the corresponding sequence of a consensus sequence provided in the “Consensus Sequence” section.

In an embodiment, the VLD comprises residue —[R₄₇]ₓ of a consensus sequence provided in the “Consensus Sequence” section, wherein x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271);
(e) a thymine hairpin domain (THD), wherein a THD comprises sufficient RNA sequence, to mediate, e.g., when present in an otherwise wildtype tRNA, recognition of the ribosome, e.g., acts as a recognition site for the ribosome to form a TREM-ribosome complex during translation. In an embodiment the THD has at least 75, 80, 85, 85, 90, 95, or 100% identity with a naturally occurring THD, e.g., a THD encoded by a nucleic acid in Table 1. In an embodiment, the TREM can comprise a fragment or analog of a THD, e.g., a THD encoded by a nucleic acid in Table 1, which fragment in embodiments has THD activity and in other embodiments does not have THD activity.

In an embodiment the THD falls under the corresponding sequence of a consensus sequence provided in the “Consensus Sequence” section, or differs from the consensus sequence by no more than 1, 2, 5, or 10 positions;

In an embodiment, the THD comprises residues —R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄ of Formula I _{ZZZ}, wherein ZZZ indicates any of the twenty amino acids;

In an embodiment, the THD comprises residues —R₄₈-R₄₉-Roo-R₅₁-R₅₂-R₅₃-R₅₄-R₅₅-R₅₆-R₅₇-R₅₈-R₅₉-R₆₀-R₆₁-R₆₂-R₆₃-R₆₄ of Formula II _{ZZZ}, wherein ZZZ indicates any of the twenty amino acids;

In an embodiment, the THD comprises residues —R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄ of Formula III _{ZZZ}, wherein ZZZ indicates any of the twenty amino acids;
(e′1) a linker comprising residue R₇₂ of a consensus sequence provided in the “Consensus Sequence” section, e.g., a Linker 4 region;
(f) under physiological conditions, it comprises a stem structure and one or a plurality of loop structures, e.g., 1, 2, or 3 loops. A loop can comprise a domain described herein, e.g., a domain selected from (a)-(e). A loop can comprise one or a plurality of domains. In an embodiment, a stem or loop structure has at least 75, 80, 85, 85, 90, 95, or 100% identity with a naturally occurring stem or loop structure, e.g., a stem or loop structure encoded by a nucleic acid in Table 1. In an embodiment, the TREM can comprise a fragment or analog of a stem or loop structure, e.g., a stem or loop structure encoded by a nucleic acid in Table 1, which fragment in embodiments has activity of a stem or loop structure, and in other embodiments does not have activity of a stem or loop structure;
(g) a tertiary structure, e.g., an L-shaped tertiary structure;
(h) adaptor function, i.e., the TREM mediates acceptance of an amino acid, e.g., its cognate amino acid and transfer of the AA in the initiation or elongation of a polypeptide chain;
(i) cognate adaptor function wherein the TREM mediates acceptance and incorporation of an amino acid (e.g., cognate amino acid) associated in nature with the anti-codon of the TREM to initiate or elongate a polypeptide chain;
(j) non-cognate adaptor function, wherein the TREM mediates acceptance and incorporation of an amino acid (e.g., non-cognate amino acid) other than the amino acid associated in nature with the anti-codon of the TREM in the initiation or elongation of a polypeptide chain;
(k) a regulatory function, e.g., an epigenetic function (e.g., gene silencing function or signaling pathway modulation function), cell fate modulation function, mRNA stability modulation function, protein stability modulation function, protein transduction modulation function, or protein compartmentalization function;
(l) a structure which allows for ribosome binding;
(m) a post-transcriptional modification, e.g., a naturally occurring post-trasncriptional modification;
(n) the ability to inhibit a functional property of a tRNA, e.g., any of properties (h)-(k) possessed by a tRNA;
(o) the ability to modulate cell fate;
(p) the ability to modulate ribosome occupancy;
(q) the ability to modulate protein translation;
(r) the ability to modulate mRNA stability;
(s) the ability to modulate protein folding and structure;
(t) the ability to modulate protein transduction or compartmentalization;
(u) the ability to modulate protein stability; or
(v) the ability to modulate a signaling pathway, e.g., a cellular signaling pathway.

In an embodiment, a TREM comprises a full-length tRNA molecule or a fragment thereof.

In an embodiment, a TREM comprises the following properties: (a)-(e).

In an embodiment, a TREM comprises the following properties: (a) and (c).

In an embodiment, a TREM comprises the following properties: (a), (c) and (h).

In an embodiment, a TREM comprises the following properties: (a), (c), (h) and (b).

In an embodiment, a TREM comprises the following properties: (a), (c), (h) and (e).

In an embodiment, a TREM comprises the following properties: (a), (c), (h), (b) and (e).

In an embodiment, a TREM comprises the following properties: (a), (c), (h), (b), (e) and (g).

In an embodiment, a TREM comprises the following properties: (a), (c), (h) and (m).

In an embodiment, a TREM comprises the following properties: (a), (c), (h), (m), and (g).

In an embodiment, a TREM comprises the following properties: (a), (c), (h), (m) and (b).

In an embodiment, a TREM comprises the following properties: (a), (c), (h), (m) and (e).

In an embodiment, a TREM comprises the following properties: (a), (c), (h), (m), (g), (b) and (e).

In an embodiment, a TREM comprises the following properties: (a), (c), (h), (m), (g), (b), (e) and (q).

In an embodiment, a TREM comprises:
(i) an amino acid attachment domain that binds an amino acid (e.g., an AStD, as described in (a) herein; and
(ii) an anticodon that binds a respective codon in an mRNA (e.g., an ACHD, as described in (c) herein).

In an embodiment the TREM comprises a flexible RNA linker which provides for covalent linkage of (i) to (ii).

In an embodiment, the TREM mediates protein translation.

In an embodiment a TREM comprises a linker, e.g., an RNA linker, e.g., a flexible RNA linker, which provides for covalent linkage between a first and a second structure or domain. In an embodiment, an RNA linker comprises at least 1, 2, 3, 4, 5, 6, 7, 8, 9, 10, 11, 12, 13, 14 or 15 ribonucleotides. A TREM can comprise one or a plurality of linkers, e.g., in embodiments a TREM comprising (a), (b), (c), (d) and (e) can have a first linker between a first and second domain, and a second linker between a third domain and another domain.

In an embodiment, the TREM comprises a sequence of Formula A: [L1]-[ASt Domain1]-[L2]-[DH Domain]-[L3]-[ACH Domain]-[VL Domain]-[TH Domain]-[L4]-[ASt Domain2].

In an embodiment, a TREM comprises an RNA sequence at least 60, 65, 70, 75, 80, 85, 90, 95, 96, 97, 98 or 99% identical with, or which differs by no more than 1, 2, 3, 4, 5, 10, 15, 20, 25, or 30 ribonucleotides from, an RNA sequence encoded by a DNA sequence listed in Table 1, or a fragment or functional fragment thereof. In an embodiment, a TREM comprises an RNA sequence encoded by a DNA sequence listed in Table 1, or a fragment or functional fragment thereof. In an embodiment, a TREM comprises an RNA sequence encoded by a DNA sequence at least 60, 65, 70, 75, 80, 85, 90, 95, 96, 97, 98 or 99% identical with a DNA sequence listed in Table 1, or a fragment or functional fragment thereof. In an embodiment, a TREM comprises a TREM domain, e.g., a domain described herein, comprising at least 60, 65, 70, 75, 80, 85, 90, 95, 96, 97, 98, or 99% identical with, or which differs by no more than 1, 2, 3, 4, 5, 10, or 15, ribonucleotides from, an RNA encoded by a DNA sequence listed in Table 1, or a fragment or a functional fragment thereof. In an embodiment, a TREM comprises a TREM domain, e.g., a domain described herein, comprising an RNA sequence encoded by DNA sequence listed in Table 1, or a fragment or functional fragment thereof. In an embodiment, a TREM comprises a TREM domain, e.g., a domain described herein, comprising an RNA sequence encoded by DNA sequence at least 60, 65, 70, 75, 80, 85, 90, 95, 96, 97, 98 or 99% identical with a DNA sequence listed in Table 1, or a fragment or functional fragment thereof.

In an embodiment, a TREM is 76-90 nucleotides in length. In embodiments, a TREM or a fragment or functional fragment thereof is between 10-90 nucleotides, between 10-80 nucleotides, between 10-70 nucleotides, between 10-60 nucleotides, between 10-50 nucleotides, between 10-40 nucleotides, between 10-30 nucleotides, between 10-20 nucleotides, between 20-90 nucleotides, between 20-80 nucleotides, 20-70 nucleotides, between 20-60 nucleotides, between 20-50 nucleotides, between 20-40 nucleotides, between 30-90 nucleotides, between 30-80 nucleotides, between 30-70 nucleotides, between 30-60 nucleotides, or between 30-50 nucleotides.

In an embodiment, a TREM is aminoacylated, e.g., charged, with an amino acid by an aminoacyl tRNA synthetase.

In an embodiment, a TREM is not charged with an amino acid, e.g., an uncharged TREM (uTREM).

In an embodiment, a TREM comprises less than a full length tRNA. In embodiments, a TREM can correspond to a naturally occurring fragment of a tRNA, or to a non-naturally occurring fragment. Exemplary fragments include: TREM halves (e.g., from a cleavage in the ACHD, e.g., in the anticodon sequence, e.g., 5′halves or 3′ halves); a 5′ fragment (e.g., a fragment comprising the 5′ end, e.g., from a cleavage in a DHD or the ACHD); a 3′ fragment (e.g., a fragment comprising the 3′ end, e.g., from a cleavage in the THD); or an internal fragment (e.g., from a cleavage in one or more of the ACHD, DHD or THD).

A “TREM core fragment,” as that term is used herein, refers to a portion of the sequence of Formula B: [L1]_{y}-[ASt Domain1]_{y}-[L2]_{y}-[DH Domain]_{y}-[L3]_{y}-[ACH Domain]ₓ-[VL Domain]_{y}-[TH Domain]_{y}-[L4]_{y}-[ASt Domain2]ₓ, wherein: x=1 and y=0 or 1.

A “TREM fragment,” as used herein, refers to a portion of a TREM, wherein the TREM comprises a sequence of Formula A: [L1]-[ASt Domain1]-[L2]-[DH Domain]-[L3]-[ACH Domain]-[VL Domain]-[TH Domain]-[L4]-[ASt Domain2].

A “cognate adaptor function TREM,” as that term is used herein, refers to a TREM which mediates initiation or elongation with the AA (the cognate AA) associated in nature with the anti-codon of the TREM.

“Decreased expression,” as that term is used herein, refers to a decrease in comparison to a reference, e.g., in the case where altered control region, or addition of an agent, results in a decreased expression of the subject product, it is decreased relative to an otherwise similar cell without the alteration or addition.

An “exogenous nucleic acid,” as that term is used herein, refers to a nucleic acid sequence that is not present in or differs by at least one nucleotide from the closest sequence in a reference cell, e.g., a cell into which the exogenous nucleic acid is introduced. In an embodiment, an exogenous nucleic acid comprises a nucleic acid that encodes a TREM.

An “exogenous TREM,” as that term is used herein, refers to a TREM that:
(a) differs by at least one nucleotide or one post transcriptional modification from the closest sequence tRNA in a reference cell, e.g., a cell into which the exogenous nucleic acid is introduced;
(b) has been introduced into a cell other than the cell in which it was transcribed;
(c) is present in a cell other than one in which it naturally occurs; or
(d) has an expression profile, e.g., level or distribution, that is non-wildtype, e.g., it is expressed at a higher level than wildtype. In an embodiment, the expression profile can be mediated by a change introduced into a nucleic acid that modulates expression or by addition of an agent that modulates expression of the RNA molecule. In an embodiment an exogenous TREM comprises 1, 2, 3 or 4 of properties (a)-(d).

A “GMP-grade composition,” as that term is used herein, refers to a composition in compliance with current good manufacturing practice (cGMP) guidelines, or other similar requirements. In an embodiment, a GMP-grade composition can be used as a pharmaceutical product.

As used herein, the terms “increasing” and “decreasing” refer to modulating that results in, respectively, greater or lesser amounts of function, expression, or activity of a particular metric relative to a reference. For example, subsequent to administration to a cell, tissue or subject of a TREM described herein, the amount of a marker of a metric (e.g., protein translation, mRNA stability, protein folding) as described herein may be increased or decreased by at least 5%, 10%, 15%, 20%, 25%, 30%, 35%, 40%, 45%, 50%, 55%, 60%, 65%, 70%, 75%, 80%, 85%, 90%, 95% or 98%, 2×, 3×, 5×, 10× or more relative to the amount of the marker prior to administration or relative to the effect of a negative control agent. The metric may be measured subsequent to administration at a time that the administration has had the recited effect, e.g., at least 12 hours, 24 hours, one week, one month, 3 months, or 6 months, after a treatment has begun.

“Increased expression,” as that term is used herein, refers to an increase in comparison to a reference, e.g., in the case where altered control region, or addition of an agent, results in an increased expression of the subject product, it is increased relative to an otherwise similar cell without the alteration or addition.

A “non-cognate adaptor function TREM,” as that term is used herein, refers to a TREM which mediates initiation or elongation with an AA (a non-cognate AA) other than the AA associated in nature with the anti-codon of the TREM. In an embodiment, a non-cognate adaptor function TREM is also referred to as a mischarged TREM (mTREM).

A “non-naturally occurring sequence,” as that term is used herein, refers to a sequence wherein an Adenine is replaced by a residue other than an analog of Adenine, a Cytosine is replaced by a residue other than an analog of Cytosine, a Guanine is replaced by a residue other than an analog of Guanine, and a Uracil is replaced by a residue other than an analog of Uracil. An analog refers to any possible derivative of the ribonucleotides, A, G, C or U. In an embodiment, a sequence having a derivative of any one of ribonucleotides A, G, C or U is a non-naturally occurring sequence.

A “pharmaceutical TREM composition,” as that term is used herein, refers to a TREM composition that is suitable for pharmaceutical use. Typically, a pharmaceutical TREM composition comprises a pharmaceutical excipient. In an embodiment the TREM will be the only active ingredient in the pharmaceutical TREM composition. In embodiments the pharmaceutical TREM composition is free, substantially free, or has less than a pharmaceutically acceptable amount, of host cell proteins, DNA, e.g., host cell DNA, endotoxins, and bacteria.

A “post-transcriptional processing,” as that term is used herein, with respect to a subject molecule, e.g., a TREM, RNA or tRNAs, refers to a covalent modification of the subject molecule. In an embodiment, the covalent modification occurs post-transcriptionally. In an embodiment, the covalent modification occurs co-transcriptionally. In an embodiment the modification is made in vivo, e.g., in a cell used to produce a TREM. In an embodiment the modification is made ex vivo, e.g., it is made on a TREM isolated or obtained from the cell which produced the TREM. In an embodiment, the post-transcriptional modification is selected from a post-transcriptional modification listed in Table 2.

A “tRNA”, as that term is used herein, refers to a naturally occurring transfer ribonucleic acid in its native state.

A “TREM composition,” as that term is used herein, refers to a composition comprising a plurality of TREMs, a plurality of TREM core fragments and/or a plurality of TREM fragments. A TREM composition can comprise one or more species of TREMs, TREM core fragments or TREM fragments. In an embodiment, the composition comprises only a single species of TREM, TREM core fragment or TREM fragment. In an embodiment, the TREM composition comprises a first TREM, TREM core fragment or TREM fragment species; and a second TREM, TREM core fragment or TREM fragment species. In an embodiment, the TREM composition comprises X TREM, TREM core fragment or TREM fragment species, wherein X=2, 3, 4, 5, 6, 7, 8, 9, or 10. In an embodiment, the TREM, TREM core fragment or TREM fragment has at least 70, 75, 80, 85, 90, or 95, or has 100%, identity with a sequence encoded by a nucleic acid in Table 1. A TREM composition can comprise one or more species of TREMs, TREM core fragments or TREM fragments. In an embodiment, the TREM composition is at least 10, 20, 30, 40, 50, 60, 70, 80, 90, 95 or 99% dry weight TREMs (for a liquid composition dry weight refers to the weight after removal of substantially all liquid, e.g., after lyophilization). In an embodiment, the composition is a liquid. In an embodiment, the composition is dry, e.g., a lyophilized material. In an embodiment, the composition is a frozen composition. In an embodiment, the composition is sterile. In an embodiment, the composition comprises at least 0.5 g, 1.0 g, 5.0 g, 10 g, 15 g, 25 g, 50 g, 100 g, 200 g, 400 g, or 500 g (e.g., as determined by dry weight) of TREM. In an embodiment, at least X % of the TREMs in a TREM composition has a non-naturally occurring modification at a selected position, and X is 80, 90, 95, 96, 97, 98, 99, or 99.5.

In an embodiment, at least X % of the TREMs in a TREM composition has a non-naturally occurring modification at a first position and a non-naturally occurring modification at a second position, and X, independently, is 80, 90, 95, 96, 97, 98, 99, or 99.5. In embodiments, the modification at the first and second position is the same. In embodiments, the modification at the first and second position are different. In embodiments, the nucleiotide at the first and second position is the same, e.g., both are adenine. In embodiments, the nucleiotide at the first and second position are different, e.g., one is adenine and one is thymine.

In an embodiment, at least X % of the TREMs in a TREM composition has a non-naturally occurring modification at a first position and less than Y % have a non-naturally occurring modification at a second position, wherein X is 80, 90, 95, 96, 97, 98, 99, or 99.5 and Y is 20, 20, 5, 2, 1, 0.1, or 0.01. In embodiments, the nucleotide at the first and second position is the same, e.g., both are adenine. In embodiments the nucleotide at the first and second position are different, e.g., one is adenine and one is thymine.

### TREM, TREM Core Fragment and TREM Fragment

A “tRNA-based effector molecule” or “TREM” refers to an RNA molecule comprising one or more of the properties described herein. A TREM can comprise a non-naturally occurring modification, e.g., as provided in Table 5. A TREM may further comprise a nucleotide modification, for example, a nucleotide substitution, nucleotide deletion, or nucleotide addition, relative to a second TREM.

In an embodiment, a TREM includes a TREM comprising a sequence of Formula A; a TREM core fragment comprising a sequence of Formula B; or a TREM fragment comprising a portion of a TREM which TREM comprises a sequence of Formula A.

In an embodiment, a TREM comprises a sequence of Formula A: [L1]-[ASt Domain1]-[L2]-[DH Domain]-[L3]-[ACH Domain]-[VL Domain]-[TH Domain]-[L4]-[ASt Domain2]. In an embodiment, [VL Domain] is optional. In an embodiment, [L1] is optional.

In an embodiment, a TREM core fragment comprises a sequence of Formula B: [L1]_{y}-[ASt Domain1]ₓ-[L2]_{y}-[DH Domain]_{y}-[L3]_{y}-[ACH Domain]ₓ-[VL Domain]_{y}-[TH Domain]_{y}-[L4]_{y}-[ASt Domain2]ₓ, wherein: x=1 and y=0 or 1. In an embodiment, y=0. In an embodiment, y=1.

In an embodiment, a TREM fragment comprises a portion of a TREM, wherein the TREM comprises a sequence of Formula A: [L1]-[ASt Domain1]-[L2]-[DH Domain]-[L3]-[ACH Domain]-[VL Domain]-[TH Domain]-[L4]-[ASt Domain2], and wherein the TREM fragment comprises: one, two, three or all or any combination of the following: a TREM half (e.g., from a cleavage in the ACH Domain, e.g., in the anticodon sequence, e.g., a 5′half or a 3′ half); a 5′ fragment (e.g., a fragment comprising the 5′ end, e.g., from a cleavage in a DH Domain or the ACH Domain); a 3′ fragment (e.g., a fragment comprising the 3′ end, e.g., from a cleavage in the TH Domain); or an internal fragment (e.g., from a cleavage in any one of the ACH Domain, DH Domain or TH Domain). Exemplary TREM fragments include TREM halves (e.g., from a cleavage in the ACHD, e.g., 5′TREM halves or 3′ TREM halves), a 5′ fragment (e.g., a fragment comprising the 5′ end, e.g., from a cleavage in a DHD or the ACHD), a 3′ fragment (e.g., a fragment comprising the 3′ end of a TREM, e.g., from a cleavage in the THD), or an internal fragment (e.g., from a cleavage in one or more of the ACHD, DHD or THD).

In an embodiment, a TREM, a TREM core fragment or a TREM fragment can be charged with an amino acid (e.g., a cognate amino acid); charged with a non-cognate amino acid (e.g., a mischarged TREM (mTREM)); or not charged with an amino acid (e.g., an uncharged TREM (uTREM)). In an embodiment, a TREM, a TREM core fragment or a TREM fragment can be charged with an amino acid selected from alanine, arginine, asparagine, aspartate, cysteine, glutamine, glutamate, glycine, histidine, isoleucine, methionine, leucine, lysine, phenylalanine, proline, serine, threonine, tryptophan, tyrosine, or valine.

In some embodiments, a non-extended anticodon is an anticodon of no more than three nucleotides. In an embodiment, a non-extended codon pairs with no more than three codon nucleotides on a nucleic acid being translated.

In an embodiment, the TREM, TREM core fragment or TREM fragment is a cognate TREM. In an embodiment, the TREM, TREM core fragment or TREM fragment is a non-cognate TREM. In an embodiment, the TREM, TREM core fragment or TREM fragment recognizes a codon provided in Table 2 or Table 3.

TABLE 2
List of codons
AAA
AAC
AAG
AAU
ACA
ACC
ACG
ACU
AGA
AGC
AGG
AGU
AUA
AUC
AUG
AUU
CAA
CAC
CAG
CAU
CCA
CCC
CCG
CCU
CGA
CGC
CGG
CGU
CUA
CUC
CUG
CUU
GAA
GAC
GAG
GAU
GCA
GCC
GCG
GCU
GGA
GGC
GGG
GGU
GUA
GUC
GUG
GUU
UAA
UAC
UAG
UAU
UCA
UCC
UCG
UCU
UGA
UGC
UGG
UGU
UUA
UUC
UUG
UUU

TABLE 3
Amino acids and corresponding codons

| Amino Acid | mRNA codons |
|---|---|
| Alanine | GCU, GCC, GCA, GCG |
| Arginine | CGU, CGC, CGA, CGG, AGA, AGG |
| Asparagine | AAU, AAC |
| Aspartate | GAU, GAC |
| Cysteine | UGU, UGC |
| Glutamate | GAA, GAG |
| Glutamine | CAA, CAG |
| Glycine | GGU, GGC, GGA, GGG |
| Histidine | CAU, CAC |
| Isoleucine | AUU, AUC, AUA |
| Leucine | UUA, UUG, CUU, CUC, CUA, CUG |
| Lysine | AAA, AAG |
| Methionine | AUG |
| Phenylalanine | UUU, UUC |
| Proline | CCU, CCC, CCA, CCG |
| Serine | UCU, UCC, UCA, UCG, AGU, AGC |
| Stop | UAA, UAG, UGA |
| Threonine | ACU, ACC, ACA, ACG |
| Tryptophan | UGG |
| Tyrosine | UAU, UAC |
| Valine | GUU, GUC, GUA, GUG |

In an embodiment, a TREM comprises a ribonucleic acid (RNA) sequence encoded by a deoxyribonucleic acid (DNA) sequence disclosed in Table 1, e.g., any one of SEQ ID NOs: 1-451 disclosed in Table 1. In an embodiment, a TREM comprises an RNA sequence at least 60%, 65%, 70%, 75%, 80%, 82%, 85%, 87%, 88%, 90%, 92%, 95%, 96%, 97%, 98%, or 99% identical to an RNA sequence encoded by a DNA sequence provided in Table 1, e.g., any one of SEQ ID NOs: 1-451 disclosed in Table 1. In an embodiment, a TREM comprises an RNA sequence encoded by a DNA sequence at least 60%, 65%, 70%, 75%, 80%, 82%, 85%, 87%, 88%, 90%, 92%, 95%, 96%, 97%, 98%, or 99% identical to a DNA sequence provided in Table 1, e.g., any one of SEQ ID NOs: 1-451 disclosed in Table 1.

In an embodiment, a TREM, a TREM core fragment, or TREM fragment comprises at least 5, 10, 15, 20, 25, or 30 consecutive nucleotides of an RNA sequence encoded by a DNA sequence disclosed in Table 1, e.g., at least 5, 10, 15, 20, 25, or 30 consecutive nucleotides of an RNA sequence encoded by any one of SEQ ID NOs: 1-451 disclosed in Table 1. In an embodiment, a TREM, a TREM core fragment, or TREM fragment comprises at least 5, 10, 15, 20, 25, or 30 consecutive nucleotides of an RNA sequence at least 60%, 65%, 70%, 75%, 80%, 82%, 85%, 87%, 88%, 90%, 92%, 95%, 96%, 97%, 98%, or 99% identical to an RNA sequence encoded by a DNA sequence provided in Table 1, e.g., any one of SEQ ID NOs: 1-451 disclosed in Table 1. In an embodiment, a TREM, a TREM core fragment, or TREM fragment comprises at least 5, 10, 15, 20, 25, or 30 consecutive nucleotides of an RNA sequence encoded by a DNA sequence at least 60%, 65%, 70%, 75%, 80%, 82%, 85%, 87%, 88%, 90%, 92%, 95%, 96%, 97%, 98%, or 99% identical to a DNA sequence provided in Table 1, e.g., any one of SEQ ID NOs: 1-451 disclosed in Table 1.

In an embodiment, a TREM core fragment or a TREM fragment comprises at least 5%, 10%, 15%, 20%, 25%, 0%, 35%, 40%, 50%, 55%, 60%, 65%, 70%, 75%, 80%, 85%, 90%, 95%, 96%, 97%, 98% or 99% of an RNA sequence encoded by a DNA sequence provided in Table 1, e.g., any one of SEQ ID NOs: 1-451 disclosed in Table 1. In an embodiment, a TREM core fragment or a TREM fragment comprises at least 5%, 10%, 15%, 20%, 25%, 30%, 35%, 40%, 50%, 55%, 60%, 65%, 70%, 75%, 80%, 85%, 90%, 95%, 96%, 97%, 98% or 99% of an RNA sequence at least 80%, 85%, 90%, 95%, 96%, 97%, 98%, or 99% identical to an RNA sequence encoded by a DNA sequence provided in Table 1, e.g., any one of SEQ ID NOs: 1-451 disclosed in Table 1. In an embodiment, a TREM core fragment or a TREM fragment comprises at least 5%, 10%, 15%, 20%, 25%, 30%, 35%, 40%, 50%, 55%, 60%, 65%, 70%, 75%, 80%, 85%, 90%, 95%, 96%, 97%, 98% or 99% of an RNA sequence encoded by a DNA sequence at least 80%, 85%, 90%, 95%, 96%, 97%, 98%, or 99% identical to a DNA sequence provided in Table 1, e.g., any one of SEQ ID NOs: 1-451 disclosed in Table 1.

In an embodiment, a TREM core fragment or a TREM fragment comprises at least 5 ribonucleotides (nt), 10 nt, 15 nt, 20 nt, 25 nt, 30 nt, 35 nt, 40 nt, 45 nt, 50 nt, 55 nt or 60 nt (but less than the full length) of an RNA sequence encoded by a DNA sequence disclosed in Table 1, e.g., any one of SEQ ID NOs: 1-451 disclosed in Table 1. In an embodiment, a TREM core fragment or a TREM fragment comprises at least 5 ribonucleotides (nt), 10 nt, 15 nt, 20 nt, 25 nt, nt, 35 nt, 40 nt, 45 nt, 50 nt, 55 nt or 60 nt (but less than the full length) of an RNA sequence which is at least 80%, 85%, 90%, 95%, 96%, 97%, 98%, 99% or 100% identical to an RNA sequence encoded by a DNA sequence provided in Table 1, e.g., any one of SEQ ID NOs: 1-451 disclosed in Table 1. In an embodiment, a TREM core fragment or a TREM fragment comprises at least 5 ribonucleotides (nt), 10 nt, 15 nt, 20 nt, 25 nt, 30 nt, 35 nt, 40 nt, 45 nt, 50 nt, 55 nt or 60 nt (but less than the full length) of an RNA sequence encoded by a DNA sequence with at least 80%, 82%, 85%, 87%, 88%, 90%, 92%, 95%, 96%, 97%, 98%, 99% or 100% identity to a DNA sequence provided in Table 1, e.g., any one of SEQ TD NOs: 1-451 disclosed in Table 1.

In an embodiment, a TREM core fragment or a TREM fragment comprises a sequence of a length of between 10-90 ribonucleotides (rnt), between 10-80 rnt, between 10-70 rnt, between 10-60 rnt, between 10-50 rnt, between 10-40 rnt, between 10-30 rnt, between 10-20 rnt, between 20-90 rnt, between 20-80 rnt, 20-70 rnt, between 20-60 rnt, between 20-50 rut, between 20-40 rnt, between 30-90 rnt, between 30-80 rnt, between 30-70 rnt, between 30-60 rnt, or between 30-50 rnt

TABLE 1
List of tRNA Sequences

| SEQ ID | | |
|---|---|---|
| NO | tRNA name | tRNA sequence |
| 1 | Ala_AGC_chr6: | GGGGGTATAGCTCAGTGGTAGAGCGCGTGC |
| | 28763741-28763812 | TTAGCATGCACGAGGTCCTGGGTTCGATCC |
| | (−) | CCAGTACCTCCA |
| 2 | Ala_AGC_chr6: | GGGGAATTAGCTCAAGTGGTAGAGCGCTTG |
| | 26687485-26687557 | CTTAGCACGCAAGAGGTAGTGGGATCGATG |
| | (+) | CCCACATTCTCCA |
| 3 | Ala_AGC_chr6: | GGGGAATTAGCTCAAATGGTAGAGCGCTCG |
| | 26572092-26572164 | CTTAGCATGCGAGAGGTAGCGGGATCGATG |
| | (−) | CCCGCATTCTCCA |
| 4 | Ala_AGC_chr6: | GGGGAATTAGCTCAAGTGGTAGAGCGCTTG |
| | 26682715-26682787 | CTTAGCATGCAAGAGGTAGTGGGATCGATG |
| | (+) | CCCACATTCTCCA |
| 5 | Ala_AGC_chr6: | GGGGAATTAGCTCAAGCGGTAGAGCGCTTG |
| | 26705606-26705678 | CTTAGCATGCAAGAGGTAGTGGGATCGATG |
| | (+) | CCCACATTCTCCA |
| 6 | Ala_AGC_chr6: | GGGGAATTAGCTCAAGTGGTAGAGCGCTTG |
| | 26673590-26673662 | CTTAGCATGCAAGAGGTAGTGGGATCAATG |
| | (+) | CCCACATTCTCCA |
| 7 | Ala_AGC_chr14: | GGGGAATTAGCTCAAGTGGTAGAGCGCTCG |
| | 89445442-89445514 | CTTAGCATGCGAGAGGTAGTGGGATCGATG |
| | (+) | CCCGCATTCTCCA |
| 8 | Ala_AGC_chr6: | GGGGAATTAGCCCAAGTGGTAGAGCGCTTG |
| | 58196623-58196695 | CTTAGCATGCAAGAGGTAGTGGGATCGATG |
| | (−) | CCCACATTCTCCA |
| 9 | Ala_AGC_chr6: | GGGGGTGTAGCTCAGTGGTAGAGCGCGTGC |
| | 28806221-28806292 | TTAGCATGCACGAGGCCCCGGGTTCAATCC |
| | (−) | CCGGCACCTCCA |
| 10 | Ala_AGC_chr6: | GGGGGTGTAGCTCAGTGGTAGAGCGCGTGC |
| | 28574933-28575004 | TTAGCATGTACGAGGTCCCGGGTTCAATCC |
| | (+) | CCGGCACCTCCA |
| 11 | Ala_AGC_chr6: | GGGGATGTAGCTCAGTGGTAGAGCGCATGC |
| | 28626014-28626085 | TTAGCATGCATGAGGTCCCGGGTTCGATCC |
| | (−) | CCAGCATCTCCA |
| 12 | Ala_AGC_chr6: | GGGGGTGTAGCTCAGTGGTAGAGCGCGTGC |
| | 28678366-28678437 | TTAGCATGCACGAGGCCCTGGGTTCAATCC |
| | (+) | CCAGCACCTCCA |
| 13 | Ala_AGC_chr6: | GGGGGTATAGCTCAGCGGTAGAGCGCGTGC |
| | 28779849-28779920 | TTAGCATGCACGAGGTCCTGGGTTCAATCC |
| | (−) | CCAATACCTCCA |
| 14 | Ala_AGC_chr6: | GGGGGTGTAGCTCAGTGGTAGAGCGCGTGC |
| | 28687481-28687552 | TTAGCATGCACGAGGCCCCGGGTTCAATCC |
| | (+) | CTGGCACCTCCA |
| 15 | Ala_AGC_chr2: | GGGGGATTAGCTCAAATGGTAGAGCGCTCG |
| | 27274082-27274154 | CTTAGCATGCGAGAGGTAGCGGGATCGATG |
| | (+) | CCCGCATCCTCCA |
| 16 | Ala_AGC_chr6: | GGGGAATTAGCTCAGGCGGTAGAGCGCTCG |
| | 26730737-26730809 | CTTAGCATGCGAGAGGTAGCGGGATCGACG |
| | (+) | CCCGCATTCTCCA |
| 17 | Ala_CGC_chr6: | GGGGATGTAGCTCAGTGGTAGAGCGCATGC |
| | 26553731-26553802 | TTCGCATGTATGAGGTCCCGGGTTCGATCC |
| | (+) | CCGGCATCTCCA |
| 18 | Ala_CGC_chr6: | GGGGATGTAGCTCAGTGGTAGAGCGCATGC |
| | 28641613-28641684 | TTCGCATGTATGAGGCCCCGGGTTCGATCC |
| | (−) | CCGGCATCTCCA |
| 19 | Ala_CGC_chr2: | GGGGATGTAGCTCAGTGGTAGAGCGCGCGC |
| | 157257281-157257352 | TTCGCATGTGTGAGGTCCCGGGTTCAATCC |
| | (+) | CCGGCATCTCCA |
| 20 | Ala_CGC_chr6: | GGGGGTGTAGCTCAGTGGTAGAGCGCGTGC |
| | 28697092-28697163 | TTCGCATGTACGAGGCCCCGGGTTCGACCC |
| | (+) | CCGGCTCCTCCA |
| 21 | Ala_TGC_chr6: | GGGGGTGTAGCTCAGTGGTAGAGCGCATGC |
| | 28757547-28757618 | TTTGCATGTATGAGGTCCCGGGTTCGATCC |
| | (−) | CCGGCACCTCCA |
| 22 | Ala_TGC_chr6: | GGGGATGTAGCTCAGTGGTAGAGCGCATGC |
| | 28611222-28611293 | TTTGCATGTATGAGGTCCCGGGTTCGATCC |
| | (+) | CCGGCATCTCCA |
| 23 | Ala_TGC_chr5: | GGGGATGTAGCTCAGTGGTAGAGCGCATGC |
| | 180633868-180633939 | TTTGCATGTATGAGGCCCCGGGTTCGATCC |
| | (+) | CCGGCATCTCCA |
| 24 | Ala_TGC_chr12: | GGGGATGTAGCTCAGTGGTAGAGCGCATGC |
| | 125424512-125424583 | TTTGCACGTATGAGGCCCCGGGTTCAATCC |
| | (+) | CCGGCATCTCCA |
| 25 | Ala_TGC_chr6: | GGGGGTGTAGCTCAGTGGTAGAGCGCATGC |
| | 28785012-28785083 | TTTGCATGTATGAGGCCTCGGGTTCGATCC |
| | (−) | CCGACACCTCCA |
| 26 | Ala_TGC_chr6: | GGGGGTGTAGCTCAGTGGTAGAGCACATGC |
| | 28726141-28726212 | TTTGCATGTGTGAGGCCCCGGGTTCGATCC |
| | (−) | CCGGCACCTCCA |
| 27 | Ala_TGC_chr6: | GGGGGTGTAGCTCAGTGGTAGAGCGCATGC |
| | 28770577-28770647 | TTTGCATGTATGAGGCCTCGGTTCGATCCC |
| | (−) | CGACACCTCCA |
| 28 | Arg_ACG_chr6: | GGGCCAGTGGCGCAATGGATAACGCGTCTG |
| | 26328368-26328440 | ACTACGGATCAGAAGATTCCAGGTTCGACT |
| | (+) | CCTGGCTGGCTCG |
| 29 | Arg_ACG_chr3: | GGGCCAGTGGCGCAATGGATAACGCGTCTG |
| | 45730491-45730563 | ACTACGGATCAGAAGATTCTAGGTTCGACT |
| | (−) | CCTGGCTGGCTCG |
| 30 | Arg_CCG_chr6: | GGCCGCGTGGCCTAATGGATAAGGCGTCTG |
| | 28710729-28710801 | ATTCCGGATCAGAAGATTGAGGGTTCGAGT |
| | (−) | CCCTTCGTGGTCG |
| 31 | Arg_CCG_chr17: | GACCCAGTGGCCTAATGGATAAGGCATCAG |
| | 66016013-66016085 | CCTCCGGAGCTGGGGATTGTGGGTTCGAGT |
| | (−) | CCCATCTGGGTCG |
| 32 | Arg_CCT_chr17: | GCCCCAGTGGCCTAATGGATAAGGCACTGG |
| | 73030001-73030073 | CCTCCTAAGCCAGGGATTGTGGGTTCGAGT |
| | (+) | CCCACCTGGGGTA |
| 33 | Arg_CCT_chr17: | GCCCCAGTGGCCTAATGGATAAGGCACTGG |
| | 73030526-73030598 | CCTCCTAAGCCAGGGATTGTGGGTTCGAGT |
| | (−) | CCCACCTGGGGTG |
| 34 | Arg_CCT_chr16: | GCCCCGGTGGCCTAATGGATAAGGCATTGG |
| | 3202901-3202973 | CCTCCTAAGCCAGGGATTGTGGGTTCGAGT |
| | (+) | CCCACCCGGGGTA |
| 35 | Arg_CCT_chr7: | GCCCCAGTGGCCTAATGGATAAGGCATTGG |
| | 139025446-139025518 | CCTCCTAAGCCAGGGATTGTGGGTTCGAGT |
| | (+) | CCCATCTGGGGTG |
| 36 | Arg_CCT_chr16: | GCCCCAGTGGCCTGATGGATAAGGTACTGG |
| | 3243918-3243990 | CCTCCTAAGCCAGGGATTGTGGGTTCGAGT |
| | (+) | TCCACCTGGGGTA |
| 37 | Arg_TCG_chr15: | GGCCGCGTGGCCTAATGGATAAGGCGTCTG |
| | 89878304-89878376 | ACTTCGGATCAGAAGATTGCAGGTTCGAGT |
| | (+) | CCTGCCGCGGTCG |
| 38 | Arg_TCG_chr6: | GACCACGTGGCCTAATGGATAAGGCGTCTG |
| | 26323046-26323118 | ACTTCGGATCAGAAGATTGAGGGTTCGAAT |
| | (+) | CCCTCCGTGGTTA |
| 39 | Arg_TCG_chr17: | GACCGCGTGGCCTAATGGATAAGGCGTCTG |
| | 73031208-73031280 | ACTTCGGATCAGAAGATTGAGGGTTCGAGT |
| | (+) | CCCTTCGTGGTCG |
| 40 | Arg_TCG_chr6: | GACCACGTGGCCTAATGGATAAGGCGTCTG |
| | 26299905-26299977 | ACTTCGGATCAGAAGATTGAGGGTTCGAAT |
| | (+) | CCCTTCGTGGTTA |
| 41 | Arg_TCG_chr6: | GACCACGTGGCCTAATGGATAAGGCGTCTG |
| | 28510891-28510963 | ACTTCGGATCAGAAGATTGAGGGTTCGAAT |
| | (−) | CCCTTCGTGGTTG |
| 42 | Arg_TCG_chr9: | GGCCGTGTGGCCTAATGGATAAGGCGTCTG |
| | 112960803-112960875 | ACTTCGGATCAAAAGATTGCAGGTTTGAGT |
| | (+) | TCTGCCACGGTCG |
| 43 | Arg_TCT_chr1: | GGCTCCGTGGCGCAATGGATAGCGCATTGG |
| | 94313129-94313213 | ACTTCTAGAGGCTGAAGGCATTCAAAGGTT |
| | (+) | CCGGGTTCGAGTCCCGGCGGAGTCG |
| 44 | Arg_TCT_chr17: | GGCTCTGTGGCGCAATGGATAGCGCATTGG |
| | 8024243-8024330 | ACTTCTAGTGACGAATAGAGCAATTCAAAG |
| | (+) | GTTGTGGGTTCGAATCCCACCAGAGTCG |
| 45 | Arg_TCT_chr9: | GGCTCTGTGGCGCAATGGATAGCGCATTGG |
| | 131102355-131102445 | ACTTCTAGCTGAGCCTAGTGTGGTCATTCA |
| | (−) | AAGGTTGTGGGTTCGAGTCCCACCAGAGTC |
| | | G |
| 46 | Arg_TCT_chr11: | GGCTCTGTGGCGCAATGGATAGCGCATTGG |
| | 59318767-59318852 | ACTTCTAGATAGTTAGAGAAATTCAAAGGT |
| | (+) | TGTGGGTTCGAGTCCCACCAGAGTCG |
| 47 | Arg_TCT_chr1: | GTCTCTGTGGCGCAATGGACGAGCGCGCTG |
| | 159111401-159111474 | GACTTCTAATCCAGAGGTTCCGGGTTCGAG |
| | (−) | TCCCGGCAGAGATG |
| 48 | Arg_TCT_chr6: | GGCTCTGTGGCGCAATGGATAGCGCATTGG |
| | 27529963-27530049 | ACTTCTAGCCTAAATCAAGAGATTCAAAGG |
| | (+) | TTGCGGGTTCGAGTCCCTCCAGAGTCG |
| 49 | Asn_GTT_chr1: | GTCTCTGTGGCGCAATCGGTTAGCGCGTTC |
| | 161510031-161510104 | GGCTGTTAACCGAAAGGTTGGTGGTTCGAT |
| | (+) | CCCACCCAGGGACG |
| 50 | Asn_GTT_chr1: | GTCTCTGTGGCGCAATCGGCTAGCGCGTTT |
| | 143879832-143879905 | GGCTGTTAACTAAAAGGTTGGCGGTTCGAA |
| | (−) | CCCACCCAGAGGCG |
| 51 | Asn_GTT_chr1: | GTCTCTGTGGTGCAATCGGTTAGCGCGTTC |
| | 144301611-144301684 | CGCTGTTAACCGAAAGCTTGGTGGTTCGAG |
| | (+) | CCCACCCAGGGATG |
| 52 | Asn_GTT_chr1: | GTCTCTGTGGCGCAATCGGCTAGCGCGTTT |
| | 149326272-149326345 | GGCTGTTAACTAAAAAGTTGGTGGTTCGAA |
| | (−) | CACACCCAGAGGCG |
| 53 | Asn_GTT_chr1: | GTCTCTGTGGCGCAATCGGTTAGCGCGTTC |
| | 148248115-148248188 | GGCTGTTAACCGAAAGGTTGGTGGTTCGAG |
| | (+) | CCCACCCAGGGACG |
| 54 | Asn_GTT_chr1: | GTCTCTGTGGCGCAATCGGTTAGCGCATTC |
| | 148598314-148598387 | GGCTGTTAACCGAAAGGTTGGTGGTTCGAG |
| | (−) | CCCACCCAGGGACG |
| 55 | Asn_GTT_chr1: | GTCTCTGTGGCGCAATCGGTTAGCGCGTTC |
| | 17216172-17216245 | GGCTGTTAACCGAAAGATTGGTGGTTCGAG |
| | (+) | CCCACCCAGGGACG |
| 56 | Asn_GTT_chr1: | GTCTCTGTGGCGCAATCGGTTAGCGCGTTC |
| | 16847080-16847153 | GGCTGTTAACTGAAAGGTTGGTGGTTCGAG |
| | (−) | CCCACCCAGGGACG |
| 57 | Asn_GTT_chr1: | GTCTCTGTGGCGCAATGGGTTAGCGCGTTC |
| | 149230570-149230643 | GGCTGTTAACCGAAAGGTTGGTGGTTCGAG |
| | (−) | CCCATCCAGGGACG |
| 58 | Asn_GTT_chr1: | GTCTCTGTGGCGTAGTCGGTTAGCGCGTTC |
| | 148000805-148000878 | GGCTGTTAACCGAAAAGTTGGTGGTTCGAG |
| | (+) | CCCACCCAGGAACG |
| 59 | Asn_GTT_chr1: | GTCTCTGTGGCGCAATCGGCTAGCGCGTTT |
| | 149711798-149711871 | GGCTGTTAACTAAAAGGTTGGTGGTTCGAA |
| | (−) | CCCACCCAGAGGCG |
| 60 | Asn_GTT_chr1: | GTCTCTGTGGCGCAATCGGTTAGCGCGTTC |
| | 145979034-145979107 | GGCTGTTAACTGAAAGGTTAGTGGTTCGAG |
| | (−) | CCCACCCGGGGACG |
| 61 | Asp_GTC_chr12: | TCCTCGTTAGTATAGTGGTTAGTATCCCCG |
| | 98897281-98897352 | CCTGTCACGCGGGAGACCGGGGTTCAATTC |
| | (+) | CCCGACGGGGAG |
| 62 | Asp_GTC_chr1: | TCCTCGTTAGTATAGTGGTGAGTATCCCCG |
| | 161410615-161410686 | CCTGTCACGCGGGAGACCGGGGTTCGATTC |
| | (−) | CCCGACGGGGAG |
| 63 | Asp_GTC_chr6: | TCCTCGTTAGTATAGTGGTGAGTGTCCCCG |
| | 27551236-27551307 | TCTGTCACGCGGGAGACCGGGGTTCGATTC |
| | (−) | CCCGACGGGGAG |
| 64 | Cys_GCA_chr7: | GGGGGCATAGCTCAGTGGTAGAGCATTTGA |
| | 149007281-149007352 | CTGCAGATCAAGAGGTCCCTGGTTCAAATC |
| | (+) | CAGGTGCCCCCT |
| 65 | Cys_GCA_chr7: | GGGGGTATAGCTCAGGGGTAGAGCATTTGA |
| | 149074601-149074672 | CTGCAGATCAAGAGGTCCCTGGTTCAAATC |
| | (−) | CAGGTGCCCCCC |
| 66 | Cys_GCA_chr7: | GGGGGTATAGCTTAGCGGTAGAGCATTTGA |
| | 149112229-149112300 | CTGCAGATCAAGAGGTCCCCGGTTCAAATC |
| | (−) | CGGGTGCCCCCT |
| 67 | Cys_GCA_chr7: | GGGGGTATAGCTTAGGGGTAGAGCATTTGA |
| | 149344046-149344117 | CTGCAGATCAAAAGGTCCCTGGTTCAAATC |
| | (−) | CAGGTGCCCCTT |
| 68 | Cys_GCA_chr7: | GGGGGTATAGCTCAGGGGTAGAGCATTTGA |
| | 149052766-149052837 | CTGCAGATCAAGAGGTCCCCAGTTCAAATC |
| | (−) | TGGGTGCCCCCT |
| 69 | Cys_GCA_chr17: | GGGGGTATAGCTCAGGGGTAGAGCATTTGA |
| | 37017937-37018008 | CTGCAGATCAAGAAGTCCCCGGTTCAAATC |
| | (−) | CGGGTGCCCCCT |
| 70 | Cys_GCA_chr7: | GGGGGTATAGCTCAGGGGTAGAGCATTTGA |
| | 149281816-149281887 | CTGCAGATCAAGAGGTCTCTGGTTCAAATC |
| | (+) | CAGGTGCCCCCT |
| 71 | Cys_GCA_chr7: | GGGGGTATAGCTCAGGGGTAGAGCACTTGA |
| | 149243631-149243702 | CTGCAGATCAAGAAGTCCTTGGTTCAAATC |
| | (+) | CAGGTGCCCCCT |
| 72 | Cys_GCA_chr7: | GGGGATATAGCTCAGGGGTAGAGCATTTGA |
| | 149388272-149388343 | CTGCAGATCAAGAGGTCCCCGGTTCAAATC |
| | (−) | CGGGTGCCCCCC |
| 73 | Cys_GCA_chr7: | GGGGGTATAGTTCAGGGGTAGAGCATTTGA |
| | 149072850-149072921 | CTGCAGATCAAGAGGTCCCTGGTTCAAATC |
| | (−) | CAGGTGCCCCCT |
| 74 | Cys_GCA_chr7: | GGGGGTATAGCTCAGGGGTAGAGCATTTGA |
| | 149310156-149310227 | CTGCAAATCAAGAGGTCCCTGATTCAAATC |
| | (−) | CAGGTGCCCCCT |
| 75 | Cys_GCA_chr4: | GGGGGTATAGCTCAGTGGTAGAGCATTTGA |
| | 124430005-124430076 | CTGCAGATCAAGAGGTCCCCGGTTCAAATC |
| | (−) | CGGGTGCCCCCT |
| 76 | Cys_GCA_chr7: | GGGCGTATAGCTCAGGGGTAGAGCATTTGA |
| | 149295046-149295117 | CTGCAGATCAAGAGGTCCCCAGTTCAAATC |
| | (+) | TGGGTGCCCCCT |
| 77 | Cys_GCA_chr7: | GGGGGTATAGCTCACAGGTAGAGCATTTGA |
| | 149361915-149361986 | CTGCAGATCAAGAGGTCCCCGGTTCAAATC |
| | (+) | TGGGTGCCCCCT |
| 78 | Cys_GCA_chr7: | GGGCGTATAGCTCAGGGGTAGAGCATTTGA |
| | 149253802-149253871 | CTGCAGATCAAGAGGTCCCCAGTTCAAATC |
| | (+) | TGGGTGCCCA |
| 79 | Cys_GCA_chr7: | GGGGGTATAGCTCACAGGTAGAGCATTTGA |
| | 149292305-149292376 | CTGCAGATCAAGAGGTCCCCGGTTCAAATC |
| | (−) | CGGTTACTCCCT |
| 80 | Cys_GCA_chr7: | GGGGGTATAGCTCAGGGGTAGAGCACTTGA |
| | 149286164-149286235 | CTGCAGATCAAGAGGTCCCTGGTTCAAATC |
| | (−) | CAGGTGCCCCCT |
| 81 | Cys_GCA_chr17: | GGGGGTATAGCTCAGTGGTAGAGCATTTGA |
| | 37025545-37025616 | CTGCAGATCAAGAGGTCCCTGGTTCAAATC |
| | (−) | CGGGTGCCCCCT |
| 82 | Cys_GCA_chr15: | GGGGGTATAGCTCAGTGGGTAGAGCATTTG |
| | 80036997-80037069 | ACTGCAGATCAAGAGGTCCCCGGTTCAAAT |
| | (+) | CCGGGTGCCCCCT |
| 83 | Cys_GCA_chr3: | GGGGGTGTAGCTCAGTGGTAGAGCATTTGA |
| | 131947944-131948015 | CTGCAGATCAAGAGGTCCCTGGTTCAAATC |
| | (−) | CAGGTGCCCCCT |
| 84 | Cys_GCA_chr1: | GGGGGTATAGCTCAGGTGGTAGAGCATTTG |
| | 93981834-93981906 | ACTGCAGATCAAGAGGTCCCCGGTTCAAAT |
| | (−) | CCGGGTGCCCCCT |
| 85 | Cys_GCA_chr14: | GGGGGTATAGCTCAGGGGTAGAGCATTTGA |
| | 73429679-73429750 | CTGCAGATCAAGAGGTCCCCGGTTCAAATC |
| | (+) | CGGGTGCCCCCT |
| 86 | Cys_GCA_chr3: | GGGGGTATAGCTCAGGGGTAGAGCATTTGA |
| | 131950642-131950713 | CTGCAGATCAAGAGGTCCCTGGTTCAAATC |
| | (−) | CAGGTGCCCCCT |
| 87 | Gln_CTG_chr6: | GGTTCCATGGTGTAATGGTTAGCACTCTGG |
| | 18836402-18836473 | ACTCTGAATCCAGCGATCCGAGTTCAAATC |
| | (+) | TCGGTGGAACCT |
| 88 | Gln_CTG_chr6: | GGTTCCATGGTGTAATGGTTAGCACTCTGG |
| | 27515531-27515602 | ACTCTGAATCCAGCGATCCGAGTTCAAGTC |
| | (−) | TCGGTGGAACCT |
| 89 | Gln_CTG_chr1: | GGTTCCATGGTGTAATGGTGAGCACTCTGG |
| | 145963304-145963375 | ACTCTGAATCCAGCGATCCGAGTTCGAGTC |
| | (+) | TCGGTGGAACCT |
| 90 | Gln_CTG_chr1: | GGTTCCATGGTGTAATGGTAAGCACTCTGG |
| | 147737382-147737453 | ACTCTGAATCCAGCGATCCGAGTTCGAGTC |
| | (−) | TCGGTGGAACCT |
| 91 | Gln_CTG_chr6: | GGTTCCATGGTGTAATGGTTAGCACTCTGG |
| | 27263212-27263283 | ACTCTGAATCCGGTAATCCGAGTTCAAATC |
| | (+) | TCGGTGGAACCT |
| 92 | Gln_CTG_chr6: | GGCCCCATGGTGTAATGGTCAGCACTCTGG |
| | 27759135-27759206 | ACTCTGAATCCAGCGATCCGAGTTCAAATC |
| | (−) | TCGGTGGGACCC |
| 93 | Gln_CTG_chr1: | GGTTCCATGGTGTAATGGTAAGCACTCTGG |
| | 147800937-147801008 | ACTCTGAATCCAGCCATCTGAGTTCGAGTC |
| | (+) | TCTGTGGAACCT |
| 94 | Gln_TTG_chr17: | GGTCCCATGGTGTAATGGTTAGCACTCTGG |
| | 47269890-47269961 | ACTTTGAATCCAGCGATCCGAGTTCAAATC |
| | (+) | TCGGTGGGACCT |
| 95 | Gln_TTG_chr6: | GGTCCCATGGTGTAATGGTTAGCACTCTGG |
| | 28557156-28557227 | ACTTTGAATCCAGCAATCCGAGTTCGAATC |
| | (+) | TCGGTGGGACCT |
| 96 | Gln_TTG_chr6: | GGCCCCATGGTGTAATGGTTAGCACTCTGG |
| | 26311424-26311495 | ACTTTGAATCCAGCGATCCGAGTTCAAATC |
| | (−) | TCGGTGGGACCT |
| 97 | Gln_TTG_chr6: | GGTCCCATGGTGTAATGGTTAGCACTCTGG |
| | 145503859-145503930 | GCTTTGAATCCAGCAATCCGAGTTCGAATC |
| | (+) | TTGGTGGGACCT |
| 98 | Glu_CTC_chr1: | TCCCTGGTGGTCTAGTGGTTAGGATTCGGC |
| | 145399233-145399304 | GCTCTCACCGCCGCGGCCCGGGTTCGATTC |
| | (−) | CCGGTCAGGGAA |
| 99 | Glu_CTC_chr1: | TCCCTGGTGGTCTAGTGGTTAGGATTCGGC |
| | 249168447-249168518 | GCTCTCACCGCCGCGGCCCGGGTTCGATTC |
| | (+) | CCGGTCAGGAAA |
| 100 | Glu_TTC_chr2: | TCCCATATGGTCTAGCGGTTAGGATTCCTG |
| | 131094701-131094772 | GTTTTCACCCAGGTGGCCCGGGTTCGACTC |
| | (−) | CCGGTATGGGAA |
| 101 | Glu_TTC_chr13: | TCCCACATGGTCTAGCGGTTAGGATTCCTG |
| | 45492062-45492133 | GTTTTCACCCAGGCGGCCCGGGTTCGACTC |
| | (−) | CCGGTGTGGGAA |
| 102 | Glu_TTC_chr1: | TCCCTGGTGGTCTAGTGGCTAGGATTCGGC |
| | 17199078-17199149 | GCTTTCACCGCCGCGGCCCGGGTTCGATTC |
| | (+) | CCGGCCAGGGAA |
| 103 | Glu_TTC_chr1: | TCCCTGGTGGTCTAGTGGCTAGGATTCGGC |
| | 16861774-16861845 | GCTTTCACCGCCGCGGCCCGGGTTCGATTC |
| | (−) | CCGGTCAGGGAA |
| 104 | Gly_CCC_chr1: | GCATTGGTGGTTCAGTGGTAGAATTCTCGC |
| | 16872434-16872504 | CTCCCACGCGGGAGACCCGGGTTCAATTCC |
| | (−) | CGGCCAATGCA |
| 105 | Gly_CCC_chr2: | GCGCCGCTGGTGTAGTGGTATCATGCAAGA |
| | 70476123-70476193 | TTCCCATTCTTGCGACCCGGGTTCGATTCC |
| | (−) | CGGGCGGCGCA |
| 106 | Gly_CCC_chr17: | GCATTGGTGGTTCAATGGTAGAATTCTCGC |
| | 19764175-19764245 | CTCCCACGCAGGAGACCCAGGTTCGATTCC |
| | (+) | TGGCCAATGCA |
| 107 | Gly_GCC_chr1: | GCATGGGTGGTTCAGTGGTAGAATTCTCGC |
| | 161413094-161413164 | CTGCCACGCGGGAGGCCCGGGTTCGATTCC |
| | (+) | CGGCCCATGCA |
| 108 | Gly_GCC_chr1: | GCATTGGTGGTTCAGTGGTAGAATTCTCGC |
| | 161493637-161493707 | CTGCCACGCGGGAGGCCCGGGTTCGATTCC |
| | (−) | CGGCCAATGCA |
| 109 | Gly_GCC_chr16: | GCATTGGTGGTTCAGTGGTAGAATTCTCGC |
| | 70812114-70812184 | CTGCCACGCGGGAGGCCCGGGTTTGATTCC |
| | (−) | CGGCCAGTGCA |
| 110 | Gly_GCC_chr1: | GCATAGGTGGTTCAGTGGTAGAATTCTTGC |
| | 161450356-161450426 | CTGCCACGCAGGAGGCCCAGGTTTGATTCC |
| | (+) | TGGCCCATGCA |
| 111 | Gly_GCC_chr16: | GCATTGGTGGTTCAGTGGTAGAATTCTCGC |
| | 70822597-70822667 | CTGCCATGCGGGCGGCCGGGCTTCGATTCC |
| | (+) | TGGCCAATGCA |
| 112 | Gly_TCC_chr19: | GCGTTGGTGGTATAGTGGTTAGCATAGCTG |
| | 4724082-4724153 | CCTTCCAAGCAGTTGACCCGGGTTCGATTC |
| | (+) | CCGGCCAACGCA |
| 113 | Gly_TCC_chr1: | GCGTTGGTGGTATAGTGGTGAGCATAGCTG |
| | 145397864-145397935 | CCTTCCAAGCAGTTGACCCGGGTTCGATTC |
| | (−) | CCGGCCAACGCA |
| 114 | Gly_TCC_chr17: | GCGTTGGTGGTATAGTGGTAAGCATAGCTG |
| | 8124866-8124937 | CCTTCCAAGCAGTTGACCCGGGTTCGATTC |
| | (+) | CCGGCCAACGCA |
| 115 | Gly_TCC_chr1: | GCGTTGGTGGTATAGTGGTGAGCATAGTTG |
| | 161409961-161410032 | CCTTCCAAGCAGTTGACCCGGGCTCGATTC |
| | (−) | CCGCCCAACGCA |
| 116 | His_GTG_chr1: | GCCGTGATCGTATAGTGGTTAGTACTCTGC |
| | 145396881-145396952 | GTTGTGGCCGCAGCAACCTCGGTTCGAATC |
| | (−) | CGAGTCACGGCA |
| 117 | His_GTG_chr1: | GCCATGATCGTATAGTGGTTAGTACTCTGC |
| | 149155828-149155899 | GCTGTGGCCGCAGCAACCTCGGTTCGAATC |
| | (−) | CGAGTCACGGCA |
| 118 | Ile_AAT_chr6: | GGCCGGTTAGCTCAGTTGGTTAGAGCGTGG |
| | 58149254-58149327 | CGCTAATAACGCCAAGGTCGCGGGTTCGAT |
| | (+) | CCCCGTACGGGCCA |
| 119 | Ile_AAT_chr6: | GGCCGGTTAGCTCAGTTGGTTAGAGCGTGG |
| | 27655967-27656040 | TGCTAATAACGCCAAGGTCGCGGGTTCGAT |
| | (+) | CCCCGTACTGGCCA |
| 120 | Ile_AAT_chr6: | GGCTGGTTAGCTCAGTTGGTTAGAGCGTGG |
| | 27242990-27243063 | TGCTAATAACGCCAAGGTCGCGGGTTCGAT |
| | (−) | CCCCGTACTGGCCA |
| 121 | Ile_AAT_chr17: | GGCCGGTTAGCTCAGTTGGTTAGAGCGTGG |
| | 8130309-8130382 | TGCTAATAACGCCAAGGTCGCGGGTTCGAA |
| | (−) | CCCCGTACGGGCCA |
| 122 | Ile_AAT_chr6: | GGCCGGTTAGCTCAGTTGGTTAGAGCGTGG |
| | 26554350-26554423 | TGCTAATAACGCCAAGGTCGCGGGTTCGAT |
| | (+) | CCCCGTACGGGCCA |
| 123 | Ile_AAT_chr6: | GGCCGGTTAGCTCAGTTGGTTAGAGCGTGG |
| | 26745255-26745328 | TGCTAATAACGCTAAGGTCGCGGGTTCGAT |
| | (−) | CCCCGTACTGGCCA |
| 124 | Ile_AAT_chr6: | GGCCGGTTAGCTCAGTTGGTCAGAGCGTGG |
| | 26721221-26721294 | TGCTAATAACGCCAAGGTCGCGGGTTCGAT |
| | (−) | CCCCGTACGGGCCA |
| 125 | Ile_AAT_chr6: | GGCCGGTTAGCTCAGTCGGCTAGAGCGTGG |
| | 27636362-27636435 | TGCTAATAACGCCAAGGTCGCGGGTTCGAT |
| | (+) | CCCCGTACGGGCCA |
| 126 | Ile_AAT_chr6: | GGCTGGTTAGTTCAGTTGGTTAGAGCGTGG |
| | 27241739-27241812 | TGCTAATAACGCCAAGGTCGTGGGTTCGAT |
| | (+) | CCCCATATCGGCCA |
| 127 | Ile_GAT_chrX: | GGCCGGTTAGCTCAGTTGGTAAGAGCGTGG |
| | 3756418-3756491 | TGCTGATAACACCAAGGTCGCGGGCTCGAC |
| | (−) | TCCCGCACCGGCCA |
| 128 | Ile_TAT_chr19: | GCTCCAGTGGCGCAATCGGTTAGCGCGCGG |
| | 39902808-39902900 | TACTTATATGACAGTGCGAGCGGAGCAATG |
| | (−) | CCGAGGTTGTGAGTTCGATCCTCACCTGGA |
| | | GCA |
| 129 | Ile_TAT_chr2: | GCTCCAGTGGCGCAATCGGTTAGCGCGCGG |
| | 43037676-43037768 | TACTTATACAGCAGTACATGCAGAGCAATG |
| | (+) | CCGAGGTTGTGAGTTCGAGCCTCACCTGGA |
| | | GCA |
| 130 | Ile_TAT_chr6: | GCTCCAGTGGCGCAATCGGTTAGCGCGCGG |
| | 26988125-26988218 | TACTTATATGGCAGTATGTGTGCGAGTGAT |
| | (+) | GCCGAGGTTGTGAGTTCGAGCCTCACCTGG |
| | | AGCA |
| 131 | Ile_TAT_chr6: | GCTCCAGTGGCGCAATCGGTTAGCGCGCGG |
| | 27599200-27599293 | TACTTATACAACAGTATATGTGCGGGTGAT |
| | (+) | GCCGAGGTTGTGAGTTCGAGCCTCACCTGG |
| | | AGCA |
| 132 | Ile_TAT_chr6: | GCTCCAGTGGCGCAATCGGTTAGCGCGCGG |
| | 28505367-28505460 | TACTTATAAGACAGTGCACCTGTGAGCAAT |
| | (+) | GCCGAGGTTGTGAGTTCAAGCCTCACCTGG |
| | | AGCA |
| 133 | Leu_AAG_chr5: | GGTAGCGTGGCCGAGCGGTCTAAGGCGCTG |
| | 180524474-180524555 | GATTAAGGCTCCAGTCTCTTCGGAGGCGTG |
| | (−) | GGTTCGAATCCCACCGCTGCCA |
| 134 | Leu_AAG_chr5: | GGTAGCGTGGCCGAGCGGTCTAAGGCGCTG |
| | 180614701-180614782 | GATTAAGGCTCCAGTCTCTTCGGGGGCGTG |
| | (+) | GGTTCGAATCCCACCGCTGCCA |
| 135 | Leu_AAG_chr6: | GGTAGCGTGGCCGAGCGGTCTAAGGCGCTG |
| | 28956779-28956860 | GATTAAGGCTCCAGTCTCTTCGGGGGCGTG |
| | (+) | GGTTCAAATCCCACCGCTGCCA |
| 136 | Leu_AAG_chr6: | GGTAGCGTGGCCGAGTGGTCTAAGACGCTG |
| | 28446400-28446481 | GATTAAGGCTCCAGTCTCTTCGGGGGCGTG |
| | (−) | GGTTTGAATCCCACCGCTGCCA |
| 137 | Leu_CAA_chr6: | GTCAGGATGGCCGAGTGGTCTAAGGCGCCA |
| | 28864000-28864105 | GACTCAAGCTAAGCTTCCTCCGCGGTGGGG |
| | (−) | ATTCTGGTCTCCAATGGAGGCGTGGGTTCG |
| | | AATCCCACTTCTGACA |
| 138 | Leu_CAA_chr6: | GTCAGGATGGCCGAGTGGTCTAAGGCGCCA |
| | 28908830-28908934 | GACTCAAGCTTGGCTTCCTCGTGTTGAGGA |
| | (+) | TTCTGGTCTCCAATGGAGGCGTGGGTTCGA |
| | | ATCCCACTTCTGACA |
| 139 | Leu_CAA_chr6: | GTCAGGATGGCCGAGTGGTCTAAGGCGCCA |
| | 27573417-27573524 | GACTCAAGCTTACTGCTTCCTGTGTTCGGG |
| | (−) | TCTTCTGGTCTCCGTATGGAGGCGTGGGTT |
| | | CGAATCCCACTTCTGACA |
| 140 | Leu_CAA_chr6: | GTCAGGATGGCCGAGTGGTCTAAGGCGCCA |
| | 27570348-27570454 | GACTCAAGTTGCTACTTCCCAGGTTTGGGG |
| | (−) | CTTCTGGTCTCCGCATGGAGGCGTGGGTTC |
| | | GAATCCCACTTCTGACA |
| 141 | Leu_CAA_chr1: | GTCAGGATGGCCGAGTGGTCTAAGGCGCCA |
| | 249168054-249168159 | GACTCAAGGTAAGCACCTTGCCTGCGGGCT |
| | (+) | TTCTGGTCTCCGGATGGAGGCGTGGGTTCG |
| | | AATCCCACTTCTGACA |
| 142 | Leu_CAA_chr11: | GCCTCCTTAGTGCAGTAGGTAGCGCATCAG |
| | 9296790-9296863 | TCTCAAAATCTGAATGGTCCTGAGTTCAAG |
| | (+) | CCTCAGAGGGGGCA |
| 143 | Leu_CAA_chr1: | GTCAGGATGGCCGAGCAGTCTTAAGGCGCT |
| | 161581736-161581819 | GCGTTCAAATCGCACCCTCCGCTGGAGGCG |
| | (−) | TGGGTTCGAATCCCACTTTTGACA |
| 144 | Leu_CAG_chr1: | GTCAGGATGGCCGAGCGGTCTAAGGCGCTG |
| | 161411323-161411405 | CGTTCAGGTCGCAGTCTCCCCTGGAGGCGT |
| | (+) | GGGTTCGAATCCCACTCCTGACA |
| 145 | Leu_CAG_chr16: | GTCAGGATGGCCGAGCGGTCTAAGGCGCTG |
| | 57333863-57333945 | CGTTCAGGTCGCAGTCTCCCCTGGAGGCGT |
| | (+) | GGGTTCGAATCCCACTTCTGACA |
| 146 | Leu_TAA_chr6: | ACCAGGATGGCCGAGTGGTTAAGGCGTTGG |
| | 144537684-144537766 | ACTTAAGATCCAATGGACATATGTCCGCGT |
| | (+) | GGGTTCGAACCCCACTCCTGGTA |
| 147 | Leu_TAA_chr6: | ACCGGGATGGCCGAGTGGTTAAGGCGTTGG |
| | 27688898-27688980 | ACTTAAGATCCAATGGGCTGGTGCCCGCGT |
| | (−) | GGGTTCGAACCCCACTCTCGGTA |
| 148 | Leu_TAA_chr11: | ACCAGAATGGCCGAGTGGTTAAGGCGTTGG |
| | 59319228-59319310 | ACTTAAGATCCAATGGATTCATATCCGCGT |
| | (+) | GGGTTCGAACCCCACTTCTGGTA |
| 149 | Leu_TAA_chr6: | ACCGGGATGGCTGAGTGGTTAAGGCGTTGG |
| | 27198334-27198416 | ACTTAAGATCCAATGGACAGGTGTCCGCGT |
| | (−) | GGGTTCGAGCCCCACTCCCGGTA |
| 150 | Leu_TAG_chr17: | GGTAGCGTGGCCGAGCGGTCTAAGGCGCTG |
| | 8023632-8023713 | GATTTAGGCTCCAGTCTCTTCGGAGGCGTG |
| | (−) | GGTTCGAATCCCACCGCTGCCA |
| 151 | Leu_TAG_chr14: | GGTAGTGTGGCCGAGCGGTCTAAGGCGCTG |
| | 21093529-21093610 | GATTTAGGCTCCAGTCTCTTCGGGGGCGTG |
| | (+) | GGTTCGAATCCCACCACTGCCA |
| 152 | Leu_TAG_chr16: | GGTAGCGTGGCCGAGTGGTCTAAGGCGCTG |
| | 22207032-22207113 | GATTTAGGCTCCAGTCATTTCGATGGCGTG |
| | (−) | GGTTCGAATCCCACCGCTGCCA |
| 153 | Lys_CTT_chr14: | GCCCGGCTAGCTCAGTCGGTAGAGCATGGG |
| | 58706613-58706685 | ACTCTTAATCCCAGGGTCGTGGGTTCGAGC |
| | (−) | CCCACGTTGGGCG |
| 154 | Lys_CTT_chr19: | GCCCAGCTAGCTCAGTCGGTAGAGCATAAG |
| | 36066750-36066822 | ACTCTTAATCTCAGGGTTGTGGATTCGTGC |
| | (+) | CCCATGCTGGGTG |
| 155 | Lys_CTT_chr19: | GCAGCTAGCTCAGTCGGTAGAGCATGAGAC |
| | 52425393-52425466 | TCTTAATCTCAGGGTCATGGGTTCGTGCCC |
| | (−) | CATGTTGGGTGCCA |
| 156 | Lys_CTT_chr1: | GCCCGGCTAGCTCAGTCGGTAGAGCATGAG |
| | 145395522-145395594 | ACTCTTAATCTCAGGGTCGTGGGTTCGAGC |
| | (−) | CCCACGTTGGGCG |
| 157 | Lys_CTT_chr16: | GCCCGGCTAGCTCAGTCGGTAGAGCATGAG |
| | 3207406-3207478 | ACCCTTAATCTCAGGGTCGTGGGTTCGAGC |
| | (−) | CCCACGTTGGGCG |
| 158 | Lys_CTT_chr16: | GCCCGGCTAGCTCAGTCGGTAGAGCATGGG |
| | 3241501-3241573 | ACTCTTAATCTCAGGGTCGTGGGTTCGAGC |
| | (+) | CCCACGTTGGGCG |
| 159 | Lys_CTT_chr16: | GCCCGGCTAGCTCAGTCGATAGAGCATGAG |
| | 3230555-3230627 | ACTCTTAATCTCAGGGTCGTGGGTTCGAGC |
| | (−) | CGCACGTTGGGCG |
| 160 | Lys_CTT_chr1: | GCCCAGCTAGCTCAGTCGGTAGAGCATGAG |
| | 55423542-55423614 | ACTCTTAATCTCAGGGTCATGGGTTTGAGC |
| | (−) | CCCACGTTTGGTG |
| 161 | Lys_CTT_chr16: | GCCTGGCTAGCTCAGTCGGCAAAGCATGAG |
| | 3214939-3215011 | ACTCTTAATCTCAGGGTCGTGGGCTCGAGC |
| | (+) | TCCATGTTGGGCG |
| 162 | Lys_CTT_chr5: | GCCCGACTACCTCAGTCGGTGGAGCATGGG |
| | 26198539-26198611 | ACTCTTCATCCCAGGGTTGTGGGTTCGAGC |
| | (−) | CCCACATTGGGCA |
| 163 | Lys_TTT_chr16: | GCCTGGATAGCTCAGTTGGTAGAGCATCAG |
| | 73512216-73512288 | ACTTTTAATCTGAGGGTCCAGGGTTCAAGT |
| | (−) | CCCTGTTCAGGCA |
| 164 | Lys_TTT_chr12: | ACCCAGATAGCTCAGTCAGTAGAGCATCAG |
| | 27843306-27843378 | ACTTTTAATCTGAGGGTCCAAGGTTCATGT |
| | (+) | CCCTTTTTGGGTG |
| 165 | Lys_TTT_chr11: | GCCTGGATAGCTCAGTTGGTAGAGCATCAG |
| | 122430655-122430727 | ACTTTTAATCTGAGGGTCCAGGGTTCAAGT |
| | (+) | CCCTGTTCAGGCG |
| 166 | Lys_TTT_chr1: | GCCCGGATAGCTCAGTCGGTAGAGCATCAG |
| | 204475655-204475727 | ACTTTTAATCTGAGGGTCCAGGGTTCAAGT |
| | (+) | CCCTGTTCGGGCG |
| 167 | Lys_TTT_chr6: | GCCTGGATAGCTCAGTCGGTAGAGCATCAG |
| | 27559593-27559665 | ACTTTTAATCTGAGGGTCCAGGGTTCAAGT |
| | (−) | CCCTGTTCAGGCG |
| 168 | Lys_TTT_chr11: | GCCCGGATAGCTCAGTCGGTAGAGCATCAG |
| | 59323902-59323974 | ACTTTTAATCTGAGGGTCCGGGGTTCAAGT |
| | (+) | CCCTGTTCGGGCG |
| 169 | Lys_TTT_chr6: | GCCTGGGTAGCTCAGTCGGTAGAGCATCAG |
| | 27302769-27302841 | ACTTTTAATCTGAGGGTCCAGGGTTCAAGT |
| | (−) | CCCTGTCCAGGCG |
| 170 | Lys_TTT_chr6: | GCCTGGATAGCTCAGTTGGTAGAACATCAG |
| | 28715521-28715593 | ACTTTTAATCTGACGGTGCAGGGTTCAAGT |
| | (+) | CCCTGTTCAGGCG |
| 171 | Met_CAT_chr8: | GCCTCGTTAGCGCAGTAGGTAGCGCGTCAG |
| | 124169470-124169542 | TCTCATAATCTGAAGGTCGTGAGTTCGATC |
| | (−) | CTCACACGGGGCA |
| 172 | Met_CAT_chr16: | GCCCTCTTAGCGCAGTGGGCAGCGCGTCAG |
| | 71460396-71460468 | TCTCATAATCTGAAGGTCCTGAGTTCGAGC |
| | (+) | CTCAGAGAGGGCA |
| 173 | Met_CAT_chr6: | GCCTCCTTAGCGCAGTAGGCAGCGCGTCAG |
| | 28912352-28912424 | TCTCATAATCTGAAGGTCCTGAGTTCGAAC |
| | (+) | CTCAGAGGGGGCA |
| 174 | Met_CAT_chr6: | GCCCTCTTAGCGCAGCGGGCAGCGCGTCAG |
| | 26735574-26735646 | TCTCATAATCTGAAGGTCCTGAGTTCGAGC |
| | (−) | CTCAGAGAGGGCA |
| 175 | Met_CAT_chr6: | GCCCTCTTAGCGCAGCTGGCAGCGCGTCAG |
| | 26701712-26701784 | TCTCATAATCTGAAGGTCCTGAGTTCAAGC |
| | (+) | CTCAGAGAGGGCA |
| 176 | Met_CAT_chr16: | GCCTCGTTAGCGCAGTAGGCAGCGCGTCAG |
| | 87417628-87417700 | TCTCATAATCTGAAGGTCGTGAGTTCGAGC |
| | (−) | CTCACACGGGGCA |
| 177 | Met_CAT_chr6: | GCCCTCTTAGTGCAGCTGGCAGCGCGTCAG |
| | 58168492-58168564 | TTTCATAATCTGAAAGTCCTGAGTTCAAGC |
| | (−) | CTCAGAGAGGGCA |
| 178 | Phe_GAA_chr6: | GCCGAAATAGCTCAGTTGGGAGAGCGTTAG |
| | 28758499-28758571 | ACTGAAGATCTAAAGGTCCCTGGTTCGATC |
| | (−) | CCGGGTTTCGGCA |
| 179 | Phe_GAA_chr11: | GCCGAAATAGCTCAGTTGGGAGAGCGTTAG |
| | 59333853-59333925 | ACTGAAGATCTAAAGGTCCCTGGTTCAATC |
| | (−) | CCGGGTTTCGGCA |
| 180 | Phe_GAA_chr6: | GCCGAGATAGCTCAGTTGGGAGAGCGTTAG |
| | 28775610-28775682 | ACTGAAGATCTAAAGGTCCCTGGTTCAATC |
| | (−) | CCGGGTTTCGGCA |
| 181 | Phe_GAA_chr6: | GCCGAAATAGCTCAGTTGGGAGAGCGTTAG |
| | 28791093-28791166 | ACCGAAGATCTTAAAGGTCCCTGGTTCAAT |
| | (−) | CCCGGGTTTCGGCA |
| 182 | Phe_GAA_chr6: | GCTGAAATAGCTCAGTTGGGAGAGCGTTAG |
| | 28731374-28731447 | ACTGAAGATCTTAAAGTTCCCTGGTTCAAC |
| | (−) | CCTGGGTTTCAGCC |
| 183 | Pro_AGG_chr16: | GGCTCGTTGGTCTAGGGGTATGATTCTCGC |
| | 3241989-3242060 | TTAGGATGCGAGAGGTCCCGGGTTCAAATC |
| | (+) | CCGGACGAGCCC |
| 184 | Pro_AGG_chr1: | GGCTCGTTGGTCTAGGGGTATGATTCTCGC |
| | 167684725-167684796 | TTAGGGTGCGAGAGGTCCCGGGTTCAAATC |
| | (−) | CCGGACGAGCCC |
| 185 | Pro_CGG_chr1: | GGCTCGTTGGTCTAGGGGTATGATTCTCGC |
| | 167683962-167684033 | TTCGGGTGCGAGAGGTCCCGGGTTCAAATC |
| | (+) | CCGGACGAGCCC |
| 186 | Pro_CGG_chr6: | GGCTCGTTGGTCTAGGGGTATGATTCTCGC |
| | 27059521-27059592 | TTCGGGTGTGAGAGGTCCCGGGTTCAAATC |
| | (+) | CCGGACGAGCCC |
| 187 | Pro_TGG_chr14: | GGCTCGTTGGTCTAGTGGTATGATTCTCGC |
| | 21101165-21101236 | TTTGGGTGCGAGAGGTCCCGGGTTCAAATC |
| | (+) | CCGGACGAGCCC |
| 188 | Pro_TGG_chr11: | GGCTCGTTGGTCTAGGGGTATGATTCTCGG |
| | 75946869-75946940 | TTTGGGTCCGAGAGGTCCCGGGTTCAAATC |
| | (−) | CCGGACGAGCCC |
| 189 | Pro_TGG_chr5: | GGCTCGTTGGTCTAGGGGTATGATTCTCGC |
| | 180615854-180615925 | TTTGGGTGCGAGAGGTCCCGGGTTCAAATC |
| | (−) | CCGGACGAGCCC |
| 190 | SeC_TCA_chr19: | GCCCGGATGATCCTCAGTGGTCTGGGGTGC |
| | 45981859-45981945 | AGGCTTCAAACCTGTAGCTGTCTAGCGACA |
| | (−) | GAGTGGTTCAATTCCACCTTTCGGGCG |
| 191 | SeC_TCA_chr22: | GCTCGGATGATCCTCAGTGGTCTGGGGTGC |
| | 44546537-44546620 | AGGCTTCAAACCTGTAGCTGTCTAGTGACA |
| | (+) | GAGTGGTTCAATTCCACCTTTGTA |
| 192 | Ser_AGA_chr6: | GTAGTCGTGGCCGAGTGGTTAAGGCGATGG |
| | 27509554-27509635 | ACTAGAAATCCATTGGGGTTTCCCCGCGCA |
| | (−) | GGTTCGAATCCTGCCGACTACG |
| 193 | Ser_AGA_chr6: | GTAGTCGTGGCCGAGTGGTTAAGGCGATGG |
| | 26327817-26327898 | ACTAGAAATCCATTGGGGTCTCCCCGCGCA |
| | (+) | GGTTCGAATCCTGCCGACTACG |
| 194 | Ser_AGA_chr6: | GTAGTCGTGGCCGAGTGGTTAAGGCGATGG |
| | 27499987-27500068 | ACTAGAAATCCATTGGGGTTTCCCCACGCA |
| | (+) | GGTTCGAATCCTGCCGACTACG |
| 195 | Ser_AGA_chr6: | GTAGTCGTGGCCGAGTGGTTAAGGTGATGG |
| | 27521192-27521273 | ACTAGAAACCCATTGGGGTCTCCCCGCGCA |
| | (−) | GGTTCGAATCCTGCCGACTACG |
| 196 | Ser_CGA_chr17: | GCTGTGATGGCCGAGTGGTTAAGGCGTTGG |
| | 8042199-8042280 | ACTCGAAATCCAATGGGGTCTCCCCGCGCA |
| | (−) | GGTTCGAATCCTGCTCACAGCG |
| 197 | Ser_CGA_chr6: | GCTGTGATGGCCGAGTGGTTAAGGCGTTGG |
| | 27177628-27177709 | ACTCGAAATCCAATGGGGTCTCCCCGCGCA |
| | (+) | GGTTCAAATCCTGCTCACAGCG |
| 198 | Ser_CGA_chr6: | GCTGTGATGGCCGAGTGGTTAAGGTGTTGG |
| | 27640229-27640310 | ACTCGAAATCCAATGGGGGTTCCCCGCGCA |
| | (−) | GGTTCAAATCCTGCTCACAGCG |
| 199 | Ser_CGA_chr12: | GTCACGGTGGCCGAGTGGTTAAGGCGTTGG |
| | 56584148-56584229 | ACTCGAAATCCAATGGGGTTTCCCCGCACA |
| | (+) | GGTTCGAATCCTGTTCGTGACG |
| 200 | Ser_GCT_chr6: | GACGAGGTGGCCGAGTGGTTAAGGCGATGG |
| | 27065085-27065166 | ACTGCTAATCCATTGTGCTCTGCACGCGTG |
| | (+) | GGTTCGAATCCCACCCTCGTCG |
| 201 | Ser_GCT_chr6: | GACGAGGTGGCCGAGTGGTTAAGGCGATGG |
| | 27265775-27265856 | ACTGCTAATCCATTGTGCTCTGCACGCGTG |
| | (+) | GGTTCGAATCCCACCTTCGTCG |
| 202 | Ser_GCT_chr11: | GACGAGGTGGCCGAGTGGTTAAGGCGATGG |
| | 66115591-66115672 | ACTGCTAATCCATTGTGCTTTGCACGCGTG |
| | (+) | GGTTCGAATCCCATCCTCGTCG |
| 203 | Ser_GCT_chr6: | GACGAGGTGGCCGAGTGGTTAAGGCGATGG |
| | 28565117-28565198 | ACTGCTAATCCATTGTGCTCTGCACGCGTG |
| | (−) | GGTTCGAATCCCATCCTCGTCG |
| 204 | Ser_GCT_chr6: | GACGAGGTGGCCGAGTGGTTAAGGCGATGG |
| | 28180815-28180896 | ACTGCTAATCCATTGTGCTCTGCACACGTG |
| | (+) | GGTTCGAATCCCATCCTCGTCG |
| 205 | Ser_GCT_chr6: | GGAGAGGCCTGGCCGAGTGGTTAAGGCGAT |
| | 26305718-26305801 | GGACTGCTAATCCATTGTGCTCTGCACGCG |
| | (−) | TGGGTTCGAATCCCATCCTCGTCG |
| 206 | Ser_TGA_chr10: | GCAGCGATGGCCGAGTGGTTAAGGCGTTGG |
| | 69524261-69524342 | ACTTGAAATCCAATGGGGTCTCCCCGCGCA |
| | (+) | GGTTCGAACCCTGCTCGCTGCG |
| 207 | Ser_TGA_chr6: | GTAGTCGTGGCCGAGTGGTTAAGGCGATGG |
| | 27513468-27513549 | ACTTGAAATCCATTGGGGTTTCCCCGCGCA |
| | (+) | GGTTCGAATCCTGCCGACTACG |
| 208 | Ser_TGA_chr6: | GTAGTCGTGGCCGAGTGGTTAAGGCGATGG |
| | 26312824-26312905 | ACTTGAAATCCATTGGGGTCTCCCCGCGCA |
| | (−) | GGTTCGAATCCTGCCGACTACG |
| 209 | Ser_TGA_chr6: | GTAGTCGTGGCCGAGTGGTTAAGGCGATGG |
| | 27473607-27473688 | ACTTGAAATCCATTGGGGTTTCCCCGCGCA |
| | (−) | GGTTCGAATCCTGTCGGCTACG |
| 210 | Thr_AGT_chr17: | GGCGCCGTGGCTTAGTTGGTTAAAGCGCCT |
| | 8090478-8090551 | GTCTAGTAAACAGGAGATCCTGGGTTCGAA |
| | (+) | TCCCAGCGGTGCCT |
| 211 | Thr_AGT_chr6: | GGCTCCGTGGCTTAGCTGGTTAAAGCGCCT |
| | 26533145-26533218 | GTCTAGTAAACAGGAGATCCTGGGTTCGAA |
| | (−) | TCCCAGCGGGGCCT |
| 212 | Thr_AGT_chr6: | GGCTCCGTAGCTTAGTTGGTTAAAGCGCCT |
| | 28693795-28693868 | GTCTAGTAAACAGGAGATCCTGGGTTCGAC |
| | (+) | TCCCAGCGGGGCCT |
| 213 | Thr_AGT_chr6: | GGCTTCGTGGCTTAGCTGGTTAAAGCGCCT |
| | 27694473-27694546 | GTCTAGTAAACAGGAGATCCTGGGTTCGAA |
| | (+) | TCCCAGCGAGGCCT |
| 214 | Thr_AGT_chr17: | GGCGCCGTGGCTTAGCTGGTTAAAGCGCCT |
| | 8042770-8042843 | GTCTAGTAAACAGGAGATCCTGGGTTCGAA |
| | (−) | TCCCAGCGGTGCCT |
| 215 | Thr_AGT_chr6: | GGCCCTGTGGCTTAGCTGGTCAAAGCGCCT |
| | 27130050-27130123 | GTCTAGTAAACAGGAGATCCTGGGTTCGAA |
| | (+) | TCCCAGCGGGGCCT |
| 216 | Thr_CGT_chr6: | GGCTCTATGGCTTAGTTGGTTAAAGCGCCT |
| | 28456770-28456843 | GTCTCGTAAACAGGAGATCCTGGGTTCGAC |
| | (−) | TCCCAGTGGGGCCT |
| 217 | Thr_CGT_chr16: | GGCGCGGTGGCCAAGTGGTAAGGCGTCGGT |
| | 14379750-14379821 | CTCGTAAACCGAAGATCACGGGTTCGAACC |
| | (+) | CCGTCCGTGCCT |
| 218 | Thr_CGT_chr6: | GGCTCTGTGGCTTAGTTGGCTAAAGCGCCT |
| | 28615984-28616057 | GTCTCGTAAACAGGAGATCCTGGGTTCGAA |
| | (−) | TCCCAGCGGGGCCT |
| 219 | Thr_CGT_chr17: | GGCGCGGTGGCCAAGTGGTAAGGCGTCGGT |
| | 29877093-29877164 | CTCGTAAACCGAAGATCGCGGGTTCGAACC |
| | (+) | CCGTCCGTGCCT |
| 220 | Thr_CGT_chr6: | GGCCCTGTAGCTCAGCGGTTGGAGCGCTGG |
| | 27586135-27586208 | TCTCGTAAACCTAGGGGTCGTGAGTTCAAA |
| | (+) | TCTCACCAGGGCCT |
| 221 | Thr_TGT_chr6: | GGCTCTATGGCTTAGTTGGTTAAAGCGCCT |
| | 28442329-28442402 | GTCTTGTAAACAGGAGATCCTGGGTTCGAA |
| | (−) | TCCCAGTAGAGCCT |
| 222 | Thr_TGT_chr1: | GGCTCCATAGCTCAGTGGTTAGAGCACTGG |
| | 222638347-222638419 | TCTTGTAAACCAGGGGTCGCGAGTTCGATC |
| | (+) | CTCGCTGGGGCCT |
| 223 | Thr_TGT_chr14: | GGCTCCATAGCTCAGGGGTTAGAGCGCTGG |
| | 21081949-21082021 | TCTTGTAAACCAGGGGTCGCGAGTTCAATT |
| | (−) | CTCGCTGGGGCCT |
| 224 | Thr_TGT_chr14: | GGCTCCATAGCTCAGGGGTTAGAGCACTGG |
| | 21099319-21099391 | TCTTGTAAACCAGGGGTCGCGAGTTCAAAT |
| | (−) | CTCGCTGGGGCCT |
| 225 | Thr_TGT_chr14: | GGCCCTATAGCTCAGGGGTTAGAGCACTGG |
| | 21149849-21149921 | TCTTGTAAACCAGGGGTCGCGAGTTCAAAT |
| | (+) | CTCGCTGGGGCCT |
| 226 | Thr_TGT_chr5: | GGCTCCATAGCTCAGGGGTTAGAGCACTGG |
| | 180618687-180618758 | TCTTGTAAACCAGGGTCGCGAGTTCAAATC |
| | (−) | TCGCTGGGGCCT |
| 227 | Trp_CCA_chr17: | GGCCTCGTGGCGCAACGGTAGCGCGTCTGA |
| | 8124187-8124258 | CTCCAGATCAGAAGGTTGCGTGTTCAAATC |
| | (−) | ACGTCGGGGTCA |
| 228 | Trp_CCA_chr17: | GACCTCGTGGCGCAATGGTAGCGCGTCTGA |
| | 19411494-19411565 | CTCCAGATCAGAAGGTTGCGTGTTCAAGTC |
| | (+) | ACGTCGGGGTCA |
| 229 | Trp_CCA_chr6: | GACCTCGTGGCGCAACGGTAGCGCGTCTGA |
| | 26319330-26319401 | CTCCAGATCAGAAGGTTGCGTGTTCAAATC |
| | (−) | ACGTCGGGGTCA |
| 230 | Trp_CCA_chr12: | GACCTCGTGGCGCAACGGTAGCGCGTCTGA |
| | 98898030-98898101 | CTCCAGATCAGAAGGCTGCGTGTTCGAATC |
| | (+) | ACGTCGGGGTCA |
| 231 | Trp_CCA_chr7: | GACCTCGTGGCGCAACGGCAGCGCGTCTGA |
| | 99067307-99067378 | CTCCAGATCAGAAGGTTGCGTGTTCAAATC |
| | (+) | ACGTCGGGGTCA |
| 232 | Tyr_ATA_chr2: | CCTTCAATAGTTCAGCTGGTAGAGCAGAGG |
| | 219110549-219110641 | ACTATAGCTACTTCCTCAGTAGGAGACGTC |
| | (+) | CTTAGGTTGCTGGTTCGATTCCAGCTTGAA |
| | | GGA |
| 233 | Tyr_GTA_chr6: | CCTTCGATAGCTCAGTTGGTAGAGCGGAGG |
| | 26569086-26569176 | ACTGTAGTTGGCTGTGTCCTTAGACATCCT |
| | (+) | TAGGTCGCTGGTTCGAATCCGGCTCGAAGG |
| | | A |
| 234 | Tyr_GTA_chr2: | CCTTCGATAGCTCAGTTGGTAGAGCGGAGG |
| | 27273650-27273738 | ACTGTAGTGGATAGGGCGTGGCAATCCTTA |
| | (+) | GGTCGCTGGTTCGATTCCGGCTCGAAGGA |
| 235 | Tyr_GTA_chr6: | CCTTCGATAGCTCAGTTGGTAGAGCGGAGG |
| | 26577332-26577420 | ACTGTAGGCTCATTAAGCAAGGTATCCTTA |
| | (+) | GGTCGCTGGTTCGAATCCGGCTCGGAGGA |
| 236 | Tyr_GTA_chr14: | CCTTCGATAGCTCAGCTGGTAGAGCGGAGG |
| | 21125623-21125716 | ACTGTAGATTGTATAGACATTTGCGGACAT |
| | (−) | CCTTAGGTCGCTGGTTCGATTCCAGCTCGA |
| | | AGGA |
| 237 | Tyr_GTA_chr8: | CCTTCGATAGCTCAGCTGGTAGAGCGGAGG |
| | 67025602-67025694 | ACTGTAGCTACTTCCTCAGCAGGAGACATC |
| | (+) | CTTAGGTCGCTGGTTCGATTCCGGCTCGAA |
| | | GGA |
| 238 | Tyr_GTA_chr8: | CCTTCGATAGCTCAGCTGGTAGAGCGGAGG |
| | 67026223-67026311 | ACTGTAGGCGCGCGCCCGTGGCCATCCTTA |
| | (+) | GGTCGCTGGTTCGATTCCGGCTCGAAGGA |
| 239 | Tyr_GTA_chr14: | CCTTCGATAGCTCAGCTGGTAGAGCGGAGG |
| | 21121258-21121351 | ACTGTAGCCTGTAGAAACATTTGTGGACAT |
| | (−) | CCTTAGGTCGCTGGTTCGATTCCGGCTCGA |
| | | AGGA |
| 240 | Tyr_GTA_chr14: | CCTTCGATAGCTCAGCTGGTAGAGCGGAGG |
| | 21131351-21131444 | ACTGTAGATTGTACAGACATTTGCGGACAT |
| | (−) | CCTTAGGTCGCTGGTTCGATTCCGGCTCGA |
| | | AGGA |
| 241 | Tyr_GTA_chr14: | CCTTCGATAGCTCAGCTGGTAGAGCGGAGG |
| | 21151432-21151520 | ACTGTAGTACTTAATGTGTGGTCATCCTTA |
| | (+) | GGTCGCTGGTTCGATTCCGGCTCGAAGGA |
| 242 | Tyr_GTA_chr6: | CCTTCGATAGCTCAGCTGGTAGAGCGGAGG |
| | 26595102-26595190 | ACTGTAGGGGTTTGAATGTGGTCATCCTTA |
| | (+) | GGTCGCTGGTTCGAATCCGGCTCGGAGGA |
| 243 | Tyr_GTA_chr14: | CCTTCGATAGCTCAGCTGGTAGAGCGGAGG |
| | 21128117-21128210 | ACTGTAGACTGCGGAAACGTTTGTGGACAT |
| | (−) | CCTTAGGTCGCTGGTTCAATTCCGGCTCGA |
| | | AGGA |
| 244 | Tyr_GTA_chr6: | CTTTCGATAGCTCAGTTGGTAGAGCGGAGG |
| | 26575798-26575887 | ACTGTAGGTTCATTAAACTAAGGCATCCTT |
| | (+) | AGGTCGCTGGTTCGAATCCGGCTCGAAGGA |
| 245 | Tyr_GTA_chr8: | TCTTCAATAGCTCAGCTGGTAGAGCGGAGG |
| | 66609532-66609619 | ACTGTAGGTGCACGCCCGTGGCCATTCTTA |
| | (−) | GGTGCTGGTTTGATTCCGACTTGGAGAG |
| 246 | Val_AAC_chr3: | GTTTCCGTAGTGTAGTGGTTATCACGTTCG |
| | 169490018-169490090 | CCTAACACGCGAAAGGTCCCCGGTTCGAAA |
| | (+) | CCGGGCGGAAACA |
| 247 | Val_AAC_chr5: | GTTTCCGTAGTGTAGTGGTCATCACGTTCG |
| | 180615416-180615488 | CCTAACACGCGAAAGGTCCCCGGTTCGAAA |
| | (−) | CCGGGCGGAAACA |
| 248 | Val_AAC_chr6: | GTTTCCGTAGTGTAGTGGTTATCACGTTCG |
| | 27618707-27618779 | CCTAACACGCGAAAGGTCCCTGGATCAAAA |
| | (−) | CCAGGCGGAAACA |
| 249 | Val_AAC_chr6: | GTTTCCGTAGTGTAGTGGTTATCACGTTCG |
| | 27648885-27648957 | CCTAACACGCGAAAGGTCCGCGGTTCGAAA |
| | (−) | CCGGGCGGAAACA |
| 250 | Val_AAC_chr6: | GTTTCCGTAGTGTAGTGGTTATCACGTTTG |
| | 27203288-27203360 | CCTAACACGCGAAAGGTCCCCGGTTCGAAA |
| | (+) | CCGGGCAGAAACA |
| 251 | Val_AAC_chr6: | GGGGGTGTAGCTCAGTGGTAGAGCGTATGC |
| | 28703206-28703277 | TTAACATTCATGAGGCTCTGGGTTCGATCC |
| | (−) | CCAGCACTTCCA |
| 252 | Val_CAC_chr1: | GTTTCCGTAGTGTAGTGGTTATCACGTTCG |
| | 161369490-161369562 | CCTCACACGCGAAAGGTCCCCGGTTCGAAA |
| | (−) | CCGGGCGGAAACA |
| 253 | Val_CAC_chr6: | GCTTCTGTAGTGTAGTGGTTATCACGTTCG |
| | 27248049-27248121 | CCTCACACGCGAAAGGTCCCCGGTTCGAAA |
| | (−) | CCGGGCAGAAGCA |
| 254 | Val_CAC_chr19: | GTTTCCGTAGTGTAGCGGTTATCACATTCG |
| | 4724647-4724719 | CCTCACACGCGAAAGGTCCCCGGTTCGATC |
| | (−) | CCGGGCGGAAACA |
| 255 | Val_CAC_chr1: | GTTTCCGTAGTGTAGTGGTTATCACGTTCG |
| | 149298555-149298627 | CCTCACACGCGAAAGGTCCCCGGTTCGAAA |
| | (−) | CTGGGCGGAAACA |
| 256 | Val_CAC_chr1: | GTTTCCGTAGTGTAGTGGTTATCACGTTCG |
| | 149684088-149684161 | CCTCACACGCGTAAAGGTCCCCGGTTCGAA |
| | (−) | ACCGGGCGGAAACA |
| 257 | Val_CAC_chr6: | GTTTCCGTAGTGGAGTGGTTATCACGTTCG |
| | 27173867-27173939 | CCTCACACGCGAAAGGTCCCCGGTTTGAAA |
| | (−) | CCAGGCGGAAACA |
| 258 | Val_TAC_chr11: | GGTTCCATAGTGTAGTGGTTATCACGTCTG |
| | 59318102-59318174 | CTTTACACGCAGAAGGTCCTGGGTTCGAGC |
| | (−) | CCCAGTGGAACCA |
| 259 | Val_TAC_chr11: | GGTTCCATAGTGTAGCGGTTATCACGTCTG |
| | 59318460-59318532 | CTTTACACGCAGAAGGTCCTGGGTTCGAGC |
| | (−) | CCCAGTGGAACCA |
| 260 | Val_TAC_chr10: | GGTTCCATAGTGTAGTGGTTATCACATCTG |
| | 5895674-5895746 | CTTTACACGCAGAAGGTCCTGGGTTCAAGC |
| | (−) | CCCAGTGGAACCA |
| 261 | Val_TAC_chr6: | GTTTCCGTGGTGTAGTGGTTATCACATTCG |
| | 27258405-27258477 | CCTTACACGCGAAAGGTCCTCGGGTCGAAA |
| | (+) | CCGAGCGGAAACA |
| 262 | iMet_CAT_chr1: | AGCAGAGTGGCGCAGCGGAAGCGTGCTGGG |
| | 153643726-153643797 | CCCATAACCCAGAGGTCGATGGATCGAAAC |
| | (+) | CATCCTCTGCTA |
| 263 | iMet_CAT_chr6: | AGCAGAGTGGCGCAGCGGAAGCGTGCTGGG |
| | 27745664-27745735 | CCCATAACCCAGAGGTCGATGGATCTAAAC |
| | (+) | CATCCTCTGCTA |
| 264 | Glu_TTC_chr1: | TCCCTGGTGGTCTAGTGGCTAGGATTCGGC |
| | 16861773-16861845 | GCTTTCACCGCCGCGGCCCGGGTTCGATTC |
| | (−) | CCGGTCAGGGAAT |
| 265 | Gly_CCC_chr1: | GCGTTGGTGGTTTAGTGGTAGAATTCTCGC |
| | 17004765-17004836 | CTCCCATGCGGGAGACCCGGGTTCAATTCC |
| | (−) | CGGCCACTGCAC |
| 266 | Gly_CCC_chr1: | GGCCTTGGTGGTGCAGTGGTAGAATTCTCG |
| | 17053779-17053850 | CCTCCCACGTGGGAGACCCGGGTTCAATTC |
| | (+) | CCGGCCAATGCA |
| 267 | Glu_TTC_chr1: | GTCCCTGGTGGTCTAGTGGCTAGGATTCGG |
| | 17199077-17199149 | CGCTTTCACCGCCGCGGCCCGGGTTCGATT |
| | (+) | CCCGGCCAGGGAA |
| 268 | Asn_GTT_chr1: | TGTCTCTGTGGCGCAATCGGTTAGCGCGTT |
| | 17216171-17216245 | CGGCTGTTAACCGAAAGATTGGTGGTTCGA |
| | (+) | GCCCACCCAGGGACG |
| 269 | Arg_TCT_chr1: | TGGCTCCGTGGCGCAATGGATAGCGCATTG |
| | 94313128-94313213 | GACTTCTAGAGGCTGAAGGCATTCAAAGGT |
| | (+) | TCCGGGTTCGAGTCCCGGCGGAGTCG |
| 270 | Lys_CTT_chr1: | GCCCGGCTAGCTCAGTCGGTAGAGCATGAG |
| | 145395521-145395594 | ACTCTTAATCTCAGGGTCGTGGGTTCGAGC |
| | (−) | CCCACGTTGGGCGC |
| 271 | His_GTG_chr1: | GCCGTGATCGTATAGTGGTTAGTACTCTGC |
| | 145396880-145396952 | GTTGTGGCCGCAGCAACCTCGGTTCGAATC |
| | (−) | CGAGTCACGGCAG |
| 272 | Gly_TCC_chr1: | GCGTTGGTGGTATAGTGGTGAGCATAGCTG |
| | 145397863-145397935 | CCTTCCAAGCAGTTGACCCGGGTTCGATTC |
| | (−) | CCGGCCAACGCAG |
| 273 | Glu_CTC_chr1: | TCCCTGGTGGTCTAGTGGTTAGGATTCGGC |
| | 145399232-145399304 | GCTCTCACCGCCGCGGCCCGGGTTCGATTC |
| | (−) | CCGGTCAGGGAAA |
| 274 | Gln_CTG_chr1: | AGGTTCCATGGTGTAATGGTGAGCACTCTG |
| | 145963303-145963375 | GACTCTGAATCCAGCGATCCGAGTTCGAGT |
| | (+) | CTCGGTGGAACCT |
| 275 | Asn_GTT_chr1: | TGTCTCTGTGGCGTAGTCGGTTAGCGCGTT |
| | 148000804-148000878 | CGGCTGTTAACCGAAAAGTTGGTGGTTCGA |
| | (+) | GCCCACCCAGGAACG |
| 276 | Asn_GTT_chr1: | TGTCTCTGTGGCGCAATCGGTTAGCGCGTT |
| | 148248114-148248188 | CGGCTGTTAACCGAAAGGTTGGTGGTTCGA |
| | (+) | GCCCACCCAGGGACG |
| 277 | Asn_GTT_chr1: | GTCTCTGTGGCGCAATCGGTTAGCGCATTC |
| | 148598313-148598387 | GGCTGTTAACCGAAAGGTTGGTGGTTCGAG |
| | (−) | CCCACCCAGGGACGC |
| 278 | Asn_GTT_chr1: | GTCTCTGTGGCGCAATGGGTTAGCGCGTTC |
| | 149230569-149230643 | GGCTGTTAACCGAAAGGTTGGTGGTTCGAG |
| | (−) | CCCATCCAGGGACGC |
| 279 | Val_CAC_chrl: | GCACTGGTGGTTCAGTGGTAGAATTCTCGC |
| | 149294665-149294736 | CTCACACGCGGGACACCCGGGTTCAATTCC |
| | (−) | CGGTCAAGGCAA |
| 280 | Val_CAC_chr1: | GTTTCCGTAGTGTAGTGGTTATCACGTTCG |
| | 149298554-149298627 | CCTCACACGCGAAAGGTCCCCGGTTCGAAA |
| | (−) | CTGGGCGGAAACAG |
| 281 | Gly_CCC_chr1: | GCACTGGTGGTTCAGTGGTAGAATTCTCGC |
| | 149680209-149680280 | CTCCCACGCGGGAGACCCGGGTTTAATTCC |
| | (−) | CGGTCAAGATAA |
| 282 | Val_CAC_chr1: | GTTTCCGTAGTGTAGTGGTTATCACGTTCG |
| | 149684087-149684161 | CCTCACACGCGTAAAGGTCCCCGGTTCGAA |
| | (−) | ACCGGGCGGAAACAT |
| 283 | Met_CAT_chr1: | TAGCAGAGTGGCGCAGCGGAAGCGTGCTGG |
| | 153643725-153643797 | GCCCATAACCCAGAGGTCGATGGATCGAAA |
| | (+) | CCATCCTCTGCTA |
| 284 | Val_CAC_chr1: | GTTTCCGTAGTGTAGTGGTTATCACGTTCG |
| | 161369489-161369562 | CCTCACACGCGAAAGGTCCCCGGTTCGAAA |
| | (−) | CCGGGCGGAAACAA |
| 285 | Asp_GTC_chr1: | TCCTCGTTAGTATAGTGGTGAGTATCCCCG |
| | 161410614-161410686 | CCTGTCACGCGGGAGACCGGGGTTCGATTC |
| | (−) | CCCGACGGGGAGG |
| 286 | Gly_GCC_chr1: | TGCATGGGTGGTTCAGTGGTAGAATTCTCG |
| | 161413093-161413164 | CCTGCCACGCGGGAGGCCCGGGTTCGATTC |
| | (+) | CCGGCCCATGCA |
| 287 | Glu_CTC_chr1: | TCCCTGGTGGTCTAGTGGTTAGGATTCGGC |
| | 161417017-161417089 | GCTCTCACCGCCGCGGCCCGGGTTCGATTC |
| | (−) | CCGGTCAGGGAAG |
| 288 | Asp_GTC_chr1: | ATCCTTGTTACTATAGTGGTGAGTATCTCT |
| | 161492934-161493006 | GCCTGTCATGCGTGAGAGAGGGGGTCGATT |
| | (+) | CCCCGACGGGGAG |
| 289 | Gly_GCC_chr1: | GCATTGGTGGTTCAGTGGTAGAATTCTCGC |
| | 161493636-161493707 | CTGCCACGCGGGAGGCCCGGGTTCGATTCC |
| | (−) | CGGCCAATGCAC |
| 290 | Leu_CAG_chr1: | GTCAGGATGGCCGAGCGGTCTAAGGCGCTG |
| | 161500131-161500214 | CGTTCAGGTCGCAGTCTCCCCTGGAGGCGT |
| | (−) | GGGTTCGAATCCCACTCCTGACAA |
| 291 | Gly_TCC_chr1: | CGCGTTGGTGGTATAGTGGTGAGCATAGCT |
| | 161500902-161500974 | GCCTTCCAAGCAGTTGACCCGGGTTCGATT |
| | (+) | CCCGGCCAACGCA |
| 292 | Asn_GTT_chr1: | CGTCTCTGTGGCGCAATCGGTTAGCGCGTT |
| | 161510030-161510104 | CGGCTGTTAACCGAAAGGTTGGTGGTTCGA |
| | (+) | TCCCACCCAGGGACG |
| 293 | Glu_TTC_chr1: | CGCGTTGGTGGTGTAGTGGTGAGCACAGCT |
| | 161582507-161582579 | GCCTTTCAAGCAGTTAACGCGGGTTCGATT |
| | (+) | CCCGGGTAACGAA |
| 294 | Pro_CGG_chr1: | CGGCTCGTTGGTCTAGGGGTATGATTCTCG |
| | 167683961-167684033 | CTTCGGGTGCGAGAGGTCCCGGGTTCAAAT |
| | (+) | CCCGGACGAGCCC |
| 295 | Pro_AGG_chr1: | GGCTCGTTGGTCTAGGGGTATGATTCTCGC |
| | 167684724-167684796 | TTAGGGTGCGAGAGGTCCCGGGTTCAAATC |
| | (−) | CCGGACGAGCCCT |
| 296 | Lys_TTT_chr1: | CGCCCGGATAGCTCAGTCGGTAGAGCATCA |
| | 204475654-204475727 | GACTTTTAATCTGAGGGTCCAGGGTTCAAG |
| | (+) | TCCCTGTTCGGGCG |
| 297 | Lys_TTT_chr1: | GCCCGGATAGCTCAGTCGGTAGAGCATCAG |
| | 204476157-204476230 | ACTTTTAATCTGAGGGTCCAGGGTTCAAGT |
| | (−) | CCCTGTTCGGGCGT |
| 298 | Leu_CAA_chr1: | TGTCAGGATGGCCGAGTGGTCTAAGGCGCC |
| | 249168053-249168159 | AGACTCAAGGTAAGCACCTTGCCTGCGGGC |
| | (+) | TTTCTGGTCTCCGGATGGAGGCGTGGGTTC |
| | | GAATCCCACTTCTGACA |
| 299 | Glu_CTC_chr1: | TTCCCTGGTGGTCTAGTGGTTAGGATTCGG |
| | 249168446-249168518 | CGCTCTCACCGCCGCGGCCCGGGTTCGATT |
| | (+) | CCCGGTCAGGAAA |
| 300 | Tyr_GTA_chr2: | GCCTTCGATAGCTCAGTTGGTAGAGCGGAG |
| | 27273649-27273738 | GACTGTAGTGGATAGGGCGTGGCAATCCTT |
| | (+) | AGGTCGCTGGTTCGATTCCGGCTCGAAGGA |
| 301 | Ala_AGC_chr2: | CGGGGGATTAGCTCAAATGGTAGAGCGCTC |
| | 27274081-27274154 | GCTTAGCATGCGAGAGGTAGCGGGATCGAT |
| | (+) | GCCCGCATCCTCCA |
| 302 | Ile_TAT_chr2: | AGCTCCAGTGGCGCAATCGGTTAGCGCGCG |
| | 43037675-43037768 | GTACTTATACAGCAGTACATGCAGAGCAAT |
| | (+) | GCCGAGGTTGTGAGTTCGAGCCTCACCTGG |
| | | AGCA |
| 303 | Gly_CCC_chr2: | GCGCCGCTGGTGTAGTGGTATCATGCAAGA |
| | 70476122-70476193 | TTCCCATTCTTGCGACCCGGGTTCGATTCC |
| | (−) | CGGGCGGCGCAT |
| 304 | Glu_TTC_chr2: | TCCCATATGGTCTAGCGGTTAGGATTCCTG |
| | 131094700-131094772 | GTTTTCACCCAGGTGGCCCGGGTTCGACTC |
| | (−) | CCGGTATGGGAAC |
| 305 | Ala_CGC_chr2: | GGGGGATGTAGCTCAGTGGTAGAGCGCGCG |
| | 157257280-157257352 | CTTCGCATGTGTGAGGTCCCGGGTTCAATC |
| | (+) | CCCGGCATCTCCA |
| 306 | Gly_GCC_chr2: | GCATTGGTGGTTCAGTGGTAGAATTCTCGC |
| | 157257658-157257729 | CTGCCACGCGGGAGGCCCGGGTTCGATTCC |
| | (−) | CGGCCAATGCAA |
| 307 | Arg_ACG_chr3: | GGGCCAGTGGCGCAATGGATAACGCGTCTG |
| | 45730490-45730563 | ACTACGGATCAGAAGATTCTAGGTTCGACT |
| | (−) | CCTGGCTGGCTCGC |
| 308 | Val_AAC_chr3: | GGTTTCCGTAGTGTAGTGGTTATCACGTTC |
| | 169490017-169490090 | GCCTAACACGCGAAAGGTCCCCGGTTCGAA |
| | (+) | ACCGGGCGGAAACA |
| 309 | Val_AAC_chr5: | AGTTTCCGTAGTGTAGTGGTTATCACGTTC |
| | 180596609-180596682 | GCCTAACACGCGAAAGGTCCCCGGTTCGAA |
| | (+) | ACCGGGCGGAAACA |
| 310 | Leu_AAG_chr5: | AGGTAGCGTGGCCGAGCGGTCTAAGGCGCT |
| | 180614700-180614782 | GGATTAAGGCTCCAGTCTCTTCGGGGGCGT |
| | (+) | GGGTTCGAATCCCACCGCTGCCA |
| 311 | Val_AAC_chr5: | GTTTCCGTAGTGTAGTGGTCATCACGTTCG |
| | 180615415-180615488 | CCTAACACGCGAAAGGTCCCCGGTTCGAAA |
| | (−) | CCGGGCGGAAACAT |
| 312 | Pro_TGG_chr5: | GGCTCGTTGGTCTAGGGGTATGATTCTCGC |
| | 180615853-180615925 | TTTGGGTGCGAGAGGTCCCGGGTTCAAATC |
| | (−) | CCGGACGAGCCCA |
| 313 | Thr_TGT_chr5: | GGCTCCATAGCTCAGGGGTTAGAGCACTGG |
| | 180618686-180618758 | TCTTGTAAACCAGGGTCGCGAGTTCAAATC |
| | (−) | TCGCTGGGGCCTG |
| 314 | Ala_TGC_chr5: | TGGGGATGTAGCTCAGTGGTAGAGCGCATG |
| | 180633867-180633939 | CTTTGCATGTATGAGGCCCCGGGTTCGATC |
| | (+) | CCCGGCATCTCCA |
| 315 | Lys_CTT_chr5: | CGCCCGGCTAGCTCAGTCGGTAGAGCATGA |
| | 180634754-180634827 | GACTCTTAATCTCAGGGTCGTGGGTTCGAG |
| | (+) | CCCCACGTTGGGCG |
| 316 | Val_AAC_chr5: | GTTTCCGTAGTGTAGTGGTTATCACGTTCG |
| | 180645269-180645342 | CCTAACACGCGAAAGGTCCCCGGTTCGAAA |
| | (−) | CCGGGCGGAAACAA |
| 317 | Lys_CTT_chr5: | GCCCGGCTAGCTCAGTCGGTAGAGCATGAG |
| | 180648978-180649051 | ACTCTTAATCTCAGGGTCGTGGGTTCGAGC |
| | (−) | CCCACGTTGGGCGT |
| 318 | Val_CAC_chr5: | GTTTCCGTAGTGTAGTGGTTATCACGTTCG |
| | 180649394-180649467 | CCTCACACGCGAAAGGTCCCCGGTTCGAAA |
| | (−) | CCGGGCGGAAACAC |
| 319 | Met_CAT_chr6: | CAGCAGAGTGGCGCAGCGGAAGCGTGCTGG |
| | 26286753-26286825 | GCCCATAACCCAGAGGTCGATGGATCGAAA |
| | (+) | CCATCCTCTGCTA |
| 320 | Ser_GCT_chr6: | GGAGAGGCCTGGCCGAGTGGTTAAGGCGAT |
| | 26305717-26305801 | GGACTGCTAATCCATTGTGCTCTGCACGCG |
| | (−) | TGGGTTCGAATCCCATCCTCGTCGC |
| 321 | Gln_TTG_chr6: | GGCCCCATGGTGTAATGGTTAGCACTCTGG |
| | 26311423-26311495 | ACTTTGAATCCAGCGATCCGAGTTCAAATC |
| | (−) | TCGGTGGGACCTG |
| 322 | Gln_TTG_chr6: | GGCCCCATGGTGTAATGGTTAGCACTCTGG |
| | 26311974-26312046 | ACTTTGAATCCAGCGATCCGAGTTCAAATC |
| | (−) | TCGGTGGGACCTA |
| 323 | Ser_TGA_chr6: | GTAGTCGTGGCCGAGTGGTTAAGGCGATGG |
| | 26312823-26312905 | ACTTGAAATCCATTGGGGTCTCCCCGCGCA |
| | (−) | GGTTCGAATCCTGCCGACTACGG |
| 324 | Met_CAT_chr6: | AGCAGAGTGGCGCAGCGGAAGCGTGCTGGG |
| | 26313351-26313423 | CCCATAACCCAGAGGTCGATGGATCGAAAC |
| | (−) | CATCCTCTGCTAT |
| 325 | Arg_TCG_chr6: | GGACCACGTGGCCTAATGGATAAGGCGTCT |
| | 26323045-26323118 | GACTTCGGATCAGAAGATTGAGGGTTCGAA |
| | (+) | TCCCTCCGTGGTTA |
| 326 | Ser_AGA_chr6: | TGTAGTCGTGGCCGAGTGGTTAAGGCGATG |
| | 26327816-26327898 | GACTAGAAATCCATTGGGGTCTCCCCGCGC |
| | (+) | AGGTTCGAATCCTGCCGACTACG |
| 327 | Met_CAT_chr6: | AGCAGAGTGGCGCAGCGGAAGCGTGCTGGG |
| | 26330528-26330600 | CCCATAACCCAGAGGTCGATGGATCGAAAC |
| | (−) | CATCCTCTGCTAG |
| 328 | Leu_CAG_chr6: | CGTCAGGATGGCCGAGCGGTCTAAGGCGCT |
| | 26521435-26521518 | GCGTTCAGGTCGCAGTCTCCCCTGGAGGCG |
| | (+) | TGGGTTCGAATCCCACTCCTGACA |
| 329 | Thr_AGT_chr6: | GGCTCCGTGGCTTAGCTGGTTAAAGCGCCT |
| | 26533144-26533218 | GTCTAGTAAACAGGAGATCCTGGGTTCGAA |
| | (−) | TCCCAGCGGGGCCTG |
| 330 | Arg_ACG_chr6: | AGGGCCAGTGGCGCAATGGATAACGCGTCT |
| | 26537725-26537798 | GACTACGGATCAGAAGATTCCAGGTTCGAC |
| | (+) | TCCTGGCTGGCTCG |
| 331 | Val_CAC_chr6: | GGTTTCCGTAGTGTAGTGGTTATCACGTTC |
| | 26538281-26538354 | GCCTCACACGCGAAAGGTCCCCGGTTCGAA |
| | (+) | ACCGGGCGGAAACA |
| 332 | Ala_CGC_chr6: | AGGGGATGTAGCTCAGTGGTAGAGCGCATG |
| | 26553730-26553802 | CTTCGCATGTATGAGGTCCCGGGTTCGATC |
| | (+) | CCCGGCATCTCCA |
| 333 | Ile_AAT_chr6: | TGGCCGGTTAGCTCAGTTGGTTAGAGCGTG |
| | 26554349-26554423 | GTGCTAATAACGCCAAGGTCGCGGGTTCGA |
| | (+) | TCCCCGTACGGGCCA |
| 334 | Pro_AGG_chr6: | CGGCTCGTTGGTCTAGGGGTATGATTCTCG |
| | 26555497-26555569 | CTTAGGGTGCGAGAGGTCCCGGGTTCAAAT |
| | (+) | CCCGGACGAGCCC |
| 335 | Lys_CTT_chr6: | AGCCCGGCTAGCTCAGTCGGTAGAGCATGA |
| | 26556773-26556846 | GACTCTTAATCTCAGGGTCGTGGGTTCGAG |
| | (+) | CCCCACGTTGGGCG |
| 336 | Tyr_GTA_chr6: | TCCTTCGATAGCTCAGTTGGTAGAGCGGAG |
| | 26569085-26569176 | GACTGTAGTTGGCTGTGTCCTTAGACATCC |
| | (+) | TTAGGTCGCTGGTTCGAATCCGGCTCGAAG |
| | | GA |
| 337 | Ala_AGC_chr6: | GGGGAATTAGCTCAAATGGTAGAGCGCTCG |
| | 26572091-26572164 | CTTAGCATGCGAGAGGTAGCGGGATCGATG |
| | (−) | CCCGCATTCTCCAG |
| 338 | Met_CAT_chr6: | CGCCCTCTTAGCGCAGCGGGCAGCGCGTCA |
| | 26766443-26766516 | GTCTCATAATCTGAAGGTCCTGAGTTCGAG |
| | (+) | CCTCAGAGAGGGCA |
| 339 | Ile_TAT_chr6: | TGCTCCAGTGGCGCAATCGGTTAGCGCGCG |
| | 26988124-26988218 | GTACTTATATGGCAGTATGTGTGCGAGTGA |
| | (+) | TGCCGAGGTTGTGAGTTCGAGCCTCACCTG |
| | | GAGCA |
| 340 | His_GTG_chr6: | TGCCGTGATCGTATAGTGGTTAGTACTCTG |
| | 27125905-27125977 | CGTTGTGGCCGCAGCAACCTCGGTTCGAAT |
| | (+) | CCGAGTCACGGCA |
| 341 | Ile_AAT_chr6: | GGCCGGTTAGCTCAGTTGGTTAGAGCGTGG |
| | 27144993-27145067 | TGCTAATAACGCCAAGGTCGCGGGTTCGAT |
| | (−) | CCCCGTACGGGCCAC |
| 342 | Val_AAC_chr6: | AGTTTCCGTAGTGTAGTGGTTATCACGTTT |
| | 27203287-27203360 | GCCTAACACGCGAAAGGTCCCCGGTTCGAA |
| | (+) | ACCGGGCAGAAACA |
| 343 | Val_CAC_chr6: | GCTTCTGTAGTGTAGTGGTTATCACGTTCG |
| | 27248048-27248121 | CCTCACACGCGAAAGGTCCCCGGTTCGAAA |
| | (−) | CCGGGCAGAAGCAA |
| 344 | Asp_GTC_chr6: | TTCCTCGTTAGTATAGTGGTGAGTATCCCC |
| | 27447452-27447524 | GCCTGTCACGCGGGAGACCGGGGTTCGATT |
| | (+) | CCCCGACGGGGAG |
| 345 | Ser_TGA_chr6: | GTAGTCGTGGCCGAGTGGTTAAGGCGATGG |
| | 27473606-27473688 | ACTTGAAATCCATTGGGGTTTCCCCGCGCA |
| | (−) | GGTTCGAATCCTGTCGGCTACGG |
| 346 | Gln_CTG_chr6: | AGGTTCCATGGTGTAATGGTTAGCACTCTG |
| | 27487307-27487379 | GACTCTGAATCCAGCGATCCGAGTTCAAAT |
| | (+) | CTCGGTGGAACCT |
| 347 | Asp_GTC_chr6: | TCCTCGTTAGTATAGTGGTGAGTGTCCCCG |
| | 27551235-27551307 | TCTGTCACGCGGGAGACCGGGGTTCGATTC |
| | (−) | CCCGACGGGGAGA |
| 348 | Val_AAC_chr6: | GTTTCCGTAGTGTAGTGGTTATCACGTTCG |
| | 27618706-27618779 | CCTAACACGCGAAAGGTCCCTGGATCAAAA |
| | (−) | CCAGGCGGAAACAA |
| 349 | Ile_AAT_chr6: | CGGCCGGTTAGCTCAGTTGGTTAGAGCGTG |
| | 27655966-27656040 | GTGCTAATAACGCCAAGGTCGCGGGTTCGA |
| | (+) | TCCCCGTACTGGCCA |
| 350 | Gln_CTG_chr6: | GGCCCCATGGTGTAATGGTCAGCACTCTGG |
| | 27759134-27759206 | ACTCTGAATCCAGCGATCCGAGTTCAAATC |
| | (−) | TCGGTGGGACCCA |
| 351 | Gln_TTG_chr6: | GGCCCCATGGTGTAATGGTTAGCACTCTGG |
| | 27763639-27763711 | ACTTTGAATCCAGCGATCCGAGTTCAAATC |
| | (−) | TCGGTGGGACCTT |
| 352 | Ala_AGC_chr6: | TGGGGGTGTAGCTCAGTGGTAGAGCGCGTG |
| | 28574932-28575004 | CTTAGCATGTACGAGGTCCCGGGTTCAATC |
| | (+) | CCCGGCACCTCCA |
| 353 | Ala_AGC_chr6: | GGGGATGTAGCTCAGTGGTAGAGCGCATGC |
| | 28626013-28626085 | TTAGCATGCATGAGGTCCCGGGTTCGATCC |
| | (−) | CCAGCATCTCCAG |
| 354 | Ala_CGC_chr6: | AGGGGGTGTAGCTCAGTGGTAGAGCGCGTG |
| | 28697091-28697163 | CTTCGCATGTACGAGGCCCCGGGTTCGACC |
| | (+) | CCCGGCTCCTCCA |
| 355 | Ala_AGC_chr6: | GGGGGTGTAGCTCAGTGGTAGAGCGCGTGC |
| | 28806220-28806292 | TTAGCATGCACGAGGCCCCGGGTTCAATCC |
| | (−) | CCGGCACCTCCAT |
| 356 | Ala_AGC_chr6: | GGGGGTGTAGCTCAGTGGTAGAGCGCGTGC |
| | 28831461-28831533 | TTAGCATGCACGAGGCCCCGGGTTCAATCC |
| | (−) | CCGGCACCTCCAG |
| 357 | Leu_CAA_chr6: | GTCAGGATGGCCGAGTGGTCTAAGGCGCCA |
| | 28863999-28864105 | GACTCAAGCTAAGCTTCCTCCGCGGTGGGG |
| | (−) | ATTCTGGTCTCCAATGGAGGCGTGGGTTCG |
| | | AATCCCACTTCTGACAC |
| 358 | Leu_CAA_chr6: | TGTCAGGATGGCCGAGTGGTCTAAGGCGCC |
| | 28908829-28908934 | AGACTCAAGCTTGGCTTCCTCGTGTTGAGG |
| | (+) | ATTCTGGTCTCCAATGGAGGCGTGGGTTCG |
| | | AATCCCACTTCTGACA |
| 359 | Gln_CTG_chr6: | GGTTCCATGGTGTAATGGTTAGCACTCTGG |
| | 28909377-28909449 | ACTCTGAATCCAGCGATCCGAGTTCAAATC |
| | (−) | TCGGTGGAACCTT |
| 360 | Leu_AAG_chr6: | GGTAGCGTGGCCGAGCGGTCTAAGGCGCTG |
| | 28911398-28911480 | GATTAAGGCTCCAGTCTCTTCGGGGGCGTG |
| | (−) | GGTTCGAATCCCACCGCTGCCAG |
| 361 | Met_CAT_chr6: | TGCCTCCTTAGCGCAGTAGGCAGCGCGTCA |
| | 28912351-28912424 | GTCTCATAATCTGAAGGTCCTGAGTTCGAA |
| | (+) | CCTCAGAGGGGGCA |
| 362 | Lys_TTT_chr6: | AGCCCGGATAGCTCAGTCGGTAGAGCATCA |
| | 28918805-28918878 | GACTTTTAATCTGAGGGTCCAGGGTTCAAG |
| | (+) | TCCCTGTTCGGGCG |
| 363 | Met_CAT_chr6: | GCCTCCTTAGCGCAGTAGGCAGCGCGTCAG |
| | 28921041-28921114 | TCTCATAATCTGAAGGTCCTGAGTTCGAAC |
| | (−) | CTCAGAGGGGGCAG |
| 364 | Glu_CTC_chr6: | TTCCCTGGTGGTCTAGTGGTTAGGATTCGG |
| | 28949975-28950047 | CGCTCTCACCGCCGCGGCCCGGGTTCGATT |
| | (+) | CCCGGTCAGGGAA |
| 365 | Leu_TAA_chr6: | CACCAGGATGGCCGAGTGGTTAAGGCGTTG |
| | 144537683-144537766 | GACTTAAGATCCAATGGACATATGTCCGCG |
| | (+) | TGGGTTCGAACCCCACTCCTGGTA |
| 366 | Pro_AGG_chr7: | TGGCTCGTTGGTCTAGGGGTATGATTCTCG |
| | 128423503-128423575 | CTTAGGGTGCGAGAGGTCCCGGGTTCAAAT |
| | (+) | CCCGGACGAGCCC |
| 367 | Arg_CCT_chr7: | AGCCCCAGTGGCCTAATGGATAAGGCATTG |
| | 139025445-139025518 | GCCTCCTAAGCCAGGGATTGTGGGTTCGAG |
| | (+) | TCCCATCTGGGGTG |
| 368 | Cys_GCA_chr7: | GGGGATATAGCTCAGGGGTAGAGCATTTGA |
| | 149388271-149388343 | CTGCAGATCAAGAGGTCCCCGGTTCAAATC |
| | (−) | CGGGTGCCCCCCC |
| 369 | Tyr_GTA_chr8: | CCCTTCGATAGCTCAGCTGGTAGAGCGGAG |
| | 67025601-67025694 | GACTGTAGCTACTTCCTCAGCAGGAGACAT |
| | (+) | CCTTAGGTCGCTGGTTCGATTCCGGCTCGA |
| | | AGGA |
| 370 | Tyr_GTA_chr8: | CCCTTCGATAGCTCAGCTGGTAGAGCGGAG |
| | 67026222-67026311 | GACTGTAGGCGCGCGCCCGTGGCCATCCTT |
| | (+) | AGGTCGCTGGTTCGATTCCGGCTCGAAGGA |
| 371 | Ala_AGC_chr8: | TGGGGGATTAGCTCAAATGGTAGAGCGCTC |
| | 67026423-67026496 | GCTTAGCATGCGAGAGGTAGCGGGATCGAT |
| | (+) | GCCCGCATCCTCCA |
| 372 | Ser_AGA_chr8: | GTAGTCGTGGCCGAGTGGTTAAGGCGATGG |
| | 96281884-96281966 | ACTAGAAATCCATTGGGGTCTCCCCGCGCA |
| | (−) | GGTTCGAATCCTGCCGACTACGG |
| 373 | Met_CAT_chr8: | GCCTCGTTAGCGCAGTAGGTAGCGCGTCAG |
| | 124169469-124169542 | TCTCATAATCTGAAGGTCGTGAGTTCGATC |
| | (−) | CTCACACGGGGCAC |
| 374 | Arg_TCT_chr9: | GGCTCTGTGGCGCAATGGATAGCGCATTGG |
| | 131102354-131102445 | ACTTCTAGCTGAGCCTAGTGTGGTCATTCA |
| | (−) | AAGGTTGTGGGTTCGAGTCCCACCAGAGTC |
| | | GA |
| 375 | Asn_GTT_chr10: | GTCTCTGTGGCGCAATCGGTTAGCGCGTTC |
| | 22518437-22518511 | GGCTGTTAACCGAAAGGTTGGTGGTTCGAG |
| | (−) | CCCACCCAGGGACGC |
| 376 | Ser_TGA_chr10: | GGCAGCGATGGCCGAGTGGTTAAGGCGTTG |
| | 69524260-69524342 | GACTTGAAATCCAATGGGGTCTCCCCGCGC |
| | (+) | AGGTTCGAACCCTGCTCGCTGCG |
| 377 | Val_TAC_chr11: | GGTTCCATAGTGTAGTGGTTATCACGTCTG |
| | 59318101-59318174 | CTTTACACGCAGAAGGTCCTGGGTTCGAGC |
| | (−) | CCCAGTGGAACCAT |
| 378 | Val_TAC_chr11: | GGTTCCATAGTGTAGCGGTTATCACGTCTG |
| | 59318459-59318532 | CTTTACACGCAGAAGGTCCTGGGTTCGAGC |
| | (−) | CCCAGTGGAACCAC |
| 379 | Arg_TCT_chr11: | TGGCTCTGTGGCGCAATGGATAGCGCATTG |
| | 59318766-59318852 | GACTTCTAGATAGTTAGAGAAATTCAAAGG |
| | (+) | TTGTGGGTTCGAGTCCCACCAGAGTCG |
| 380 | Leu_TAA_chr11: | TACCAGAATGGCCGAGTGGTTAAGGCGTTG |
| | 59319227-59319310 | GACTTAAGATCCAATGGATTCATATCCGCG |
| | (+) | TGGGTTCGAACCCCACTTCTGGTA |
| 381 | Lys_TTT_chr11: | GGCCCGGATAGCTCAGTCGGTAGAGCATCA |
| | 59323901-59323974 | GACTTTTAATCTGAGGGTCCGGGGTTCAAG |
| | (+) | TCCCTGTTCGGGCG |
| 382 | Phe_GAA_chr11: | GCCGAAATAGCTCAGTTGGGAGAGCGTTAG |
| | 59324969-59325042 | ACTGAAGATCTAAAGGTCCCTGGTTCGATC |
| | (−) | CCGGGTTTCGGCAG |
| 383 | Lys_TTT_chr11: | GCCCGGATAGCTCAGTCGGTAGAGCATCAG |
| | 59327807-59327880 | ACTTTTAATCTGAGGGTCCAGGGTTCAAGT |
| | (−) | CCCTGTTCGGGCGG |
| 384 | Phe_GAA_chr11: | GCCGAAATAGCTCAGTTGGGAGAGCGTTAG |
| | 59333852-59333925 | ACTGAAGATCTAAAGGTCCCTGGTTCAATC |
| | (−) | CCGGGTTTCGGCAG |
| 385 | Ser_GCT_chr11: | GGACGAGGTGGCCGAGTGGTTAAGGCGATG |
| | 66115590-66115672 | GACTGCTAATCCATTGTGCTTTGCACGCGT |
| | (+) | GGGTTCGAATCCCATCCTCGTCG |
| 386 | Pro_TGG_chr11: | GGCTCGTTGGTCTAGGGGTATGATTCTCGG |
| | 75946868-75946940 | TTTGGGTCCGAGAGGTCCCGGGTTCAAATC |
| | (−) | CCGGACGAGCCCC |
| 387 | Ser_CGA_chr12: | AGTCACGGTGGCCGAGTGGTTAAGGCGTTG |
| | 56584147-56584229 | GACTCGAAATCCAATGGGGTTTCCCCGCAC |
| | (+) | AGGTTCGAATCCTGTTCGTGACG |
| 388 | Asp_GTC_chr12: | CTCCTCGTTAGTATAGTGGTTAGTATCCCC |
| | 98897280-98897352 | GCCTGTCACGCGGGAGACCGGGGTTCAATT |
| | (+) | CCCCGACGGGGAG |
| 389 | Trp_CCA_chr12: | GGACCTCGTGGCGCAACGGTAGCGCGTCTG |
| | 98898029-98898101 | ACTCCAGATCAGAAGGCTGCGTGTTCGAAT |
| | (+) | CACGTCGGGGTCA |
| 390 | Ala_TGC_chr12: | GGGGATGTAGCTCAGTGGTAGAGCGCATGC |
| | 125406300-125406372 | TTTGCATGTATGAGGCCCCGGGTTCGATCC |
| | (−) | CCGGCATCTCCAT |
| 391 | Phe_GAA_chr12: | GCCGAAATAGCTCAGTTGGGAGAGCGTTAG |
| | 125412388-125412461 | ACTGAAGATCTAAAGGTCCCTGGTTCGATC |
| | (−) | CCGGGTTTCGGCAC |
| 392 | Ala_TGC_chr12: | AGGGGATGTAGCTCAGTGGTAGAGCGCATG |
| | 125424511-125424583 | CTTTGCACGTATGAGGCCCCGGGTTCAATC |
| | (+) | CCCGGCATCTCCA |
| 393 | Asn_GTT_chr13: | GTCTCTGTGGCGCAATCGGTTAGCGCGTTC |
| | 31248100-31248174 | GGCTGTTAACCGAAAGGTTGGTGGTTCGAG |
| | (−) | CCCACCCAGGGACGG |
| 394 | Glu_TTC_chr13: | TCCCACATGGTCTAGCGGTTAGGATTCCTG |
| | 45492061-45492133 | GTTTTCACCCAGGCGGCCCGGGTTCGACTC |
| | (−) | CCGGTGTGGGAAC |
| 395 | Thr_TGT_chr14: | GGCTCCATAGCTCAGGGGTTAGAGCGCTGG |
| | 21081948-21082021 | TCTTGTAAACCAGGGGTCGCGAGTTCAATT |
| | (−) | CTCGCTGGGGCCTG |
| 396 | Leu_TAG_chr14: | TGGTAGTGTGGCCGAGCGGTCTAAGGCGCT |
| | 21093528-21093610 | GGATTTAGGCTCCAGTCTCTTCGGGGGCGT |
| | (+) | GGGTTCGAATCCCACCACTGCCA |
| 397 | Thr_TGT_chr14: | GGCTCCATAGCTCAGGGGTTAGAGCACTGG |
| | 21099318-21099391 | TCTTGTAAACCAGGGGTCGCGAGTTCAAAT |
| | (−) | CTCGCTGGGGCCTC |
| 398 | Pro_TGG_chr14: | TGGCTCGTTGGTCTAGTGGTATGATTCTCG |
| | 21101164-21101236 | CTTTGGGTGCGAGAGGTCCCGGGTTCAAAT |
| | (+) | CCCGGACGAGCCC |
| 399 | Tyr_GTA_chr14: | CCTTCGATAGCTCAGCTGGTAGAGCGGAGG |
| | 21131350-21131444 | ACTGTAGATTGTACAGACATTTGCGGACAT |
| | (−) | CCTTAGGTCGCTGGTTCGATTCCGGCTCGA |
| | | AGGAA |
| 400 | Thr_TGT_chr14: | AGGCCCTATAGCTCAGGGGTTAGAGCACTG |
| | 21149848-21149921 | GTCTTGTAAACCAGGGGTCGCGAGTTCAAA |
| | (+) | TCTCGCTGGGGCCT |
| 401 | Tyr_GTA_chr14: | TCCTTCGATAGCTCAGCTGGTAGAGCGGAG |
| | 21151431-21151520 | GACTGTAGTACTTAATGTGTGGTCATCCTT |
| | (+) | AGGTCGCTGGTTCGATTCCGGCTCGAAGGA |
| 402 | Pro_TGG_chr14: | TGGCTCGTTGGTCTAGGGGTATGATTCTCG |
| | 21152174-21152246 | CTTTGGGTGCGAGAGGTCCCGGGTTCAAAT |
| | (+) | CCCGGACGAGCCC |
| 403 | Lys_CTT_chr14: | GCCCGGCTAGCTCAGTCGGTAGAGCATGGG |
| | 58706612-58706685 | ACTCTTAATCCCAGGGTCGTGGGTTCGAGC |
| | (−) | CCCACGTTGGGCGC |
| 404 | Ile_AAT_chr14: | CGGCCGGTTAGCTCAGTTGGTTAGAGCGTG |
| | 102783428-102783502 | GTGCTAATAACGCCAAGGTCGCGGGTTCGA |
| | (+) | TCCCCGTACGGGCCA |
| 405 | Glu_TTC_chr15: | TCCCACATGGTCTAGCGGTTAGGATTCCTG |
| | 26327380-26327452 | GTTTTCACCCAGGCGGCCCGGGTTCGACTC |
| | (−) | CCGGTGTGGGAAT |
| 406 | Ser_GCT_chr15: | GACGAGGTGGCCGAGTGGTTAAGGCGATGG |
| | 40886022-40886104 | ACTGCTAATCCATTGTGCTCTGCACGCGTG |
| | (−) | GGTTCGAATCCCATCCTCGTCGA |
| 407 | His_GTG_chr15: | GCCGTGATCGTATAGTGGTTAGTACTCTGC |
| | 45490803-45490875 | GTTGTGGCCGCAGCAACCTCGGTTCGAATC |
| | (−) | CGAGTCACGGCAT |
| 408 | His_GTG_chr15: | CGCCGTGATCGTATAGTGGTTAGTACTCTG |
| | 45493348-45493420 | CGTTGTGGCCGCAGCAACCTCGGTTCGAAT |
| | (+) | CCGAGTCACGGCA |
| 409 | Gln_CTG_chr15: | GGTTCCATGGTGTAATGGTTAGCACTCTGG |
| | 66161399-66161471 | ACTCTGAATCCAGCGATCCGAGTTCAAATC |
| | (−) | TCGGTGGAACCTG |
| 410 | Lys_CTT_chr15: | TGCCCGGCTAGCTCAGTCGGTAGAGCATGG |
| | 79152903-79152976 | GACTCTTAATCCCAGGGTCGTGGGTTCGAG |
| | (+) | CCCCACGTTGGGCG |
| 411 | Arg_TCG_chr15: | GGGCCGCGTGGCCTAATGGATAAGGCGTCT |
| | 89878303-89878376 | GACTTCGGATCAGAAGATTGCAGGTTCGAG |
| | (+) | TCCTGCCGCGGTCG |
| 412 | Gly_CCC_chr16: | GCGCCGCTGGTGTAGTGGTATCATGCAAGA |
| | 686735-686806 | TTCCCATTCTTGCGACCCGGGTTCGATTCC |
| | (−) | CGGGCGGCGCAC |
| 413 | Arg_CCG_chr16: | GGGCCGCGTGGCCTAATGGATAAGGCGTCT |
| | 3200674-3200747 | GATTCCGGATCAGAAGATTGAGGGTTCGAG |
| | (+) | TCCCTTCGTGGTCG |
| 414 | Arg_CCT_chr16: | CGCCCCGGTGGCCTAATGGATAAGGCATTG |
| | 3202900-3202973 | GCCTCCTAAGCCAGGGATTGTGGGTTCGAG |
| | (+) | TCCCACCCGGGGTA |
| 415 | Lys_CTT_chr16: | GCCCGGCTAGCTCAGTCGGTAGAGCATGAG |
| | 3207405-3207478 | ACCCTTAATCTCAGGGTCGTGGGTTCGAGC |
| | (−) | CCCACGTTGGGCGT |
| 416 | Thr_CGT_chr16: | AGGCGCGGTGGCCAAGTGGTAAGGCGTCGG |
| | 14379749-14379821 | TCTCGTAAACCGAAGATCACGGGTTCGAAC |
| | (+) | CCCGTCCGTGCCT |
| 417 | Leu_TAG_chr16: | GGTAGCGTGGCCGAGTGGTCTAAGGCGCTG |
| | 22207031-22207113 | GATTTAGGCTCCAGTCATTTCGATGGCGTG |
| | (−) | GGTTCGAATCCCACCGCTGCCAC |
| 418 | Leu_AAG_chr16: | GGGTAGCGTGGCCGAGCGGTCTAAGGCGCT |
| | 22308460-22308542 | GGATTAAGGCTCCAGTCTCTTCGGGGGCGT |
| | (+) | GGGTTCGAATCCCACCGCTGCCA |
| 419 | Leu_CAG_chr16: | AGTCAGGATGGCCGAGCGGTCTAAGGCGCT |
| | 57333862-57333945 | GCGTTCAGGTCGCAGTCTCCCCTGGAGGCG |
| | (+) | TGGGTTCGAATCCCACTTCTGACA |
| 420 | Leu_CAG_chr16: | GTCAGGATGGCCGAGCGGTCTAAGGCGCTG |
| | 57334391-57334474 | CGTTCAGGTCGCAGTCTCCCCTGGAGGCGT |
| | (−) | GGGTTCGAATCCCACTTCTGACAG |
| 421 | Met_CAT_chr16: | GCCTCGTTAGCGCAGTAGGCAGCGCGTCAG |
| | 87417627-87417700 | TCTCATAATCTGAAGGTCGTGAGTTCGAGC |
| | (−) | CTCACACGGGGCAG |
| 422 | Leu_TAG_chr17: | GGTAGCGTGGCCGAGCGGTCTAAGGCGCTG |
| | 8023631-8023713 | GATTTAGGCTCCAGTCTCTTCGGAGGCGTG |
| | (−) | GGTTCGAATCCCACCGCTGCCAG |
| 423 | Arg_TCT_chr17: | TGGCTCTGTGGCGCAATGGATAGCGCATTG |
| | 8024242-8024330 | GACTTCTAGTGACGAATAGAGCAATTCAAA |
| | (+) | GGTTGTGGGTTCGAATCCCACCAGAGTCG |
| 424 | Gly_GCC_chr17: | CGCATTGGTGGTTCAGTGGTAGAATTCTCG |
| | 8029063-8029134 | CCTGCCACGCGGGAGGCCCGGGTTCGATTC |
| | (+) | CCGGCCAATGCA |
| 425 | Ser_CGA_chr17: | GCTGTGATGGCCGAGTGGTTAAGGCGTTGG |
| | 8042198-8042280 | ACTCGAAATCCAATGGGGTCTCCCCGCGCA |
| | (−) | GGTTCGAATCCTGCTCACAGCGT |
| 426 | Thr_AGT_chr17: | GGCGCCGTGGCTTAGCTGGTTAAAGCGCCT |
| | 8042769-8042843 | GTCTAGTAAACAGGAGATCCTGGGTTCGAA |
| | (−) | TCCCAGCGGTGCCTG |
| 427 | Trp_CCA_chr17: | CGACCTCGTGGCGCAACGGTAGCGCGTCTG |
| | 8089675-8089747 | ACTCCAGATCAGAAGGTTGCGTGTTCAAAT |
| | (+) | CACGTCGGGGTCA |
| 428 | Ser_GCT_chr17: | AGACGAGGTGGCCGAGTGGTTAAGGCGATG |
| | 8090183-8090265 | GACTGCTAATCCATTGTGCTCTGCACGCGT |
| | (+) | GGGTTCGAATCCCATCCTCGTCG |
| 429 | Thr_AGT_chr17: | CGGCGCCGTGGCTTAGTTGGTTAAAGCGCC |
| | 8090477-8090551 | TGTCTAGTAAACAGGAGATCCTGGGTTCGA |
| | (+) | ATCCCAGCGGTGCCT |
| 430 | Trp_CCA_chr17: | GGCCTCGTGGCGCAACGGTAGCGCGTCTGA |
| | 8124186-8124258 | CTCCAGATCAGAAGGTTGCGTGTTCAAATC |
| | (−) | ACGTCGGGGTCAA |
| 431 | Gly_TCC_chr17: | AGCGTTGGTGGTATAGTGGTAAGCATAGCT |
| | 8124865-8124937 | GCCTTCCAAGCAGTTGACCCGGGTTCGATT |
| | (+) | CCCGGCCAACGCA |
| 432 | Asp_GTC_chr17: | TCCTCGTTAGTATAGTGGTGAGTATCCCCG |
| | 8125555-8125627 | CCTGTCACGCGGGAGACCGGGGTTCGATTC |
| | (−) | CCCGACGGGGAGA |
| 433 | Pro_CGG_chr17: | GGCTCGTTGGTCTAGGGGTATGATTCTCGC |
| | 8126150-8126222 | TTCGGGTGCGAGAGGTCCCGGGTTCAAATC |
| | (−) | CCGGACGAGCCCT |
| 434 | Thr_AGT_chr17: | GGCGCCGTGGCTTAGTTGGTTAAAGCGCCT |
| | 8129552-8129626 | GTCTAGTAAACAGGAGATCCTGGGTTCGAA |
| | (−) | TCCCAGCGGTGCCTT |
| 435 | Ser_AGA_chr17: | GTAGTCGTGGCCGAGTGGTTAAGGCGATGG |
| | 8129927-8130009 | ACTAGAAATCCATTGGGGTCTCCCCGCGCA |
| | (−) | GGTTCGAATCCTGCCGACTACGT |
| 436 | Trp_CCA_chr17: | TGACCTCGTGGCGCAATGGTAGCGCGTCTG |
| | 19411493-19411565 | ACTCCAGATCAGAAGGTTGCGTGTTCAAGT |
| | (+) | CACGTCGGGGTCA |
| 437 | Thr_CGT_chr17: | AGGCGCGGTGGCCAAGTGGTAAGGCGTCGG |
| | 29877092-29877164 | TCTCGTAAACCGAAGATCGCGGGTTCGAAC |
| | (+) | CCCGTCCGTGCCT |
| 438 | Cys_GCA_chr17: | AGGGGGTATAGCTCAGTGGTAGAGCATTTG |
| | 37023897-37023969 | ACTGCAGATCAAGAGGTCCCCGGTTCAAAT |
| | (+) | CCGGGTGCCCCCT |
| 439 | Cys_GCA_chr17: | GGGGGTATAGCTCAGTGGTAGAGCATTTGA |
| | 37025544-37025616 | CTGCAGATCAAGAGGTCCCTGGTTCAAATC |
| | (−) | CGGGTGCCCCCTC |
| 440 | Cys_GCA_chr17: | GGGGGTATAGCTCAGTGGTAGAGCATTTGA |
| | 37309986-37310058 | CTGCAGATCAAGAGGTCCCCGGTTCAAATC |
| | (−) | CGGGTGCCCCCTC |
| 441 | Gln_TTG_chr17: | AGGTCCCATGGTGTAATGGTTAGCACTCTG |
| | 47269889-47269961 | GACTTTGAATCCAGCGATCCGAGTTCAAAT |
| | (+) | CTCGGTGGGACCT |
| 442 | Arg_CCG_chr17: | GACCCAGTGGCCTAATGGATAAGGCATCAG |
| | 66016012-66016085 | CCTCCGGAGCTGGGGATTGTGGGTTCGAGT |
| | (−) | CCCATCTGGGTCGC |
| 443 | Arg_CCT_chr17: | AGCCCCAGTGGCCTAATGGATAAGGCACTG |
| | 73030000-73030073 | GCCTCCTAAGCCAGGGATTGTGGGTTCGAG |
| | (+) | TCCCACCTGGGGTA |
| 444 | Arg_CCT_chr17: | GCCCCAGTGGCCTAATGGATAAGGCACTGG |
| | 73030525-73030598 | CCTCCTAAGCCAGGGATTGTGGGTTCGAGT |
| | (−) | CCCACCTGGGGTGT |
| 445 | Arg_TCG_chr17: | AGACCGCGTGGCCTAATGGATAAGGCGTCT |
| | 73031207-73031280 | GACTTCGGATCAGAAGATTGAGGGTTCGAG |
| | (+) | TCCCTTCGTGGTCG |
| 446 | Asn_GTT_chr19: | CGTCTCTGTGGCGCAATCGGTTAGCGCGTT |
| | 1383561-1383635 | CGGCTGTTAACCGAAAGGTTGGTGGTTCGA |
| | (+) | GCCCACCCAGGGACG |
| 447 | Gly_TCC_chr19: | GGCGTTGGTGGTATAGTGGTTAGCATAGCT |
| | 4724081-4724153 | GCCTTCCAAGCAGTTGACCCGGGTTCGATT |
| | (+) | CCCGGCCAACGCA |
| 448 | Val_CAC_chr19: | GTTTCCGTAGTGTAGCGGTTATCACATTCG |
| | 4724646-4724719 | CCTCACACGCGAAAGGTCCCCGGTTCGATC |
| | (−) | CCGGGCGGAAACAG |
| 449 | Thr_AGT_chr19: | TGGCGCCGTGGCTTAGTTGGTTAAAGCGCC |
| | 33667962-33668036 | TGTCTAGTAAACAGGAGATCCTGGGTTCGA |
| | (+) | ATCCCAGCGGTGCCT |
| 450 | Ile_TAT_chr19: | GCTCCAGTGGCGCAATCGGTTAGCGCGCGG |
| | 39902807-39902900 | TACTTATATGACAGTGCGAGCGGAGCAATG |
| | (−) | CCGAGGTTGTGAGTTCGATCCTCACCTGGA |
| | | GCAC |
| 451 | Gly_GCC_chr21: | GCATGGGTGGTTCAGTGGTAGAATTCTCGC |
| | 18827106-18827177 | CTGCCACGCGGGAGGCCCGGGTTCGATTCC |
| | (−) | CGGCCCATGCAG |

### Nucleotide Sequence Modifications

The present disclosure describes TREMs comprising a nucleotide sequence modification, e.g., relative to a first TREM nucleotide sequence, that may modulate a functional parameter of the TREM. These TREMs may further comprise a non-naturally occurring chemical modification, or may not. For example, a TREM comprising a nucleotide sequence modification may exhibit improved activity or stability, e.g., in vitro or in a cell. The nucleotide sequence modification may be a nucleotide substitution, e.g., a change of a nucleotide at a given position in the TREM sequence to a different nucleotide. In an embodiment, the nucleotide substitution is an A to U substitution. In an embodiment, the nucleotide substitution is an A to G substitution. In an embodiment, the nucleotide substitution is an A to C substitution. In an embodiment, the nucleotide substitution is a U to A substitution. In an embodiment, the nucleotide substitution is a U to G substitution. In an embodiment, the nucleotide substitution is a U to C substitution. In an embodiment, the nucleotide substitution is a G to A substitution. In an embodiment, the nucleotide substitution is a G to U substitution. In an embodiment, the nucleotide substitution is a G to C substitution. In an embodiment, the nucleotide substitution is a C to A substitution. In an embodiment, the nucleotide substitution is a C to U substitution. In an embodiment, the nucleotide substitution is a C to G substitution.

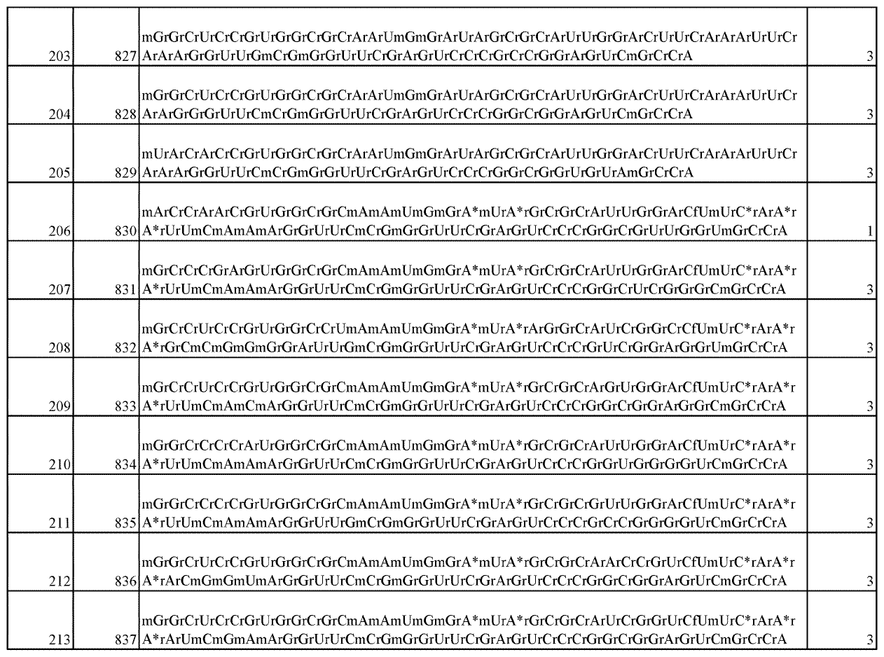

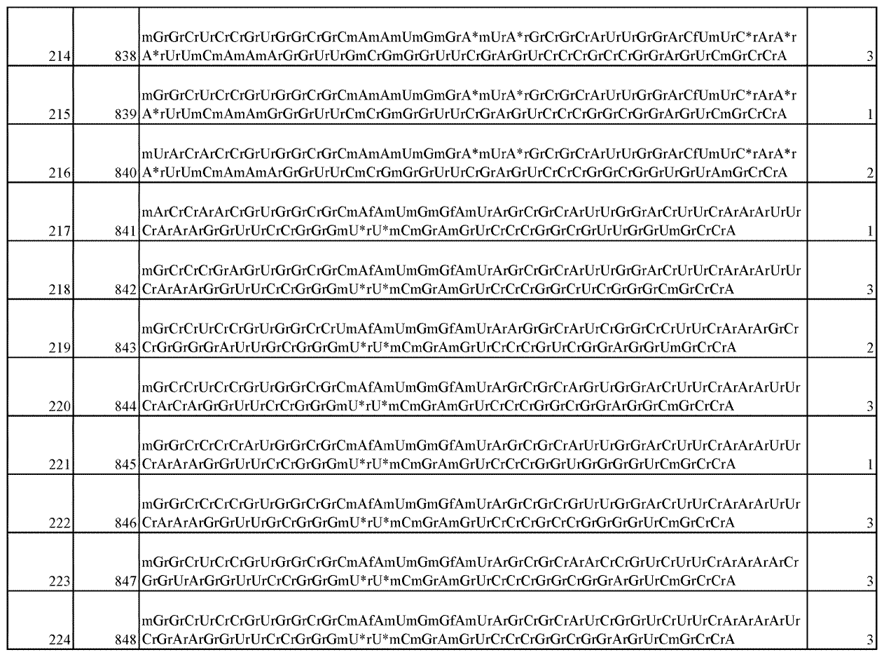

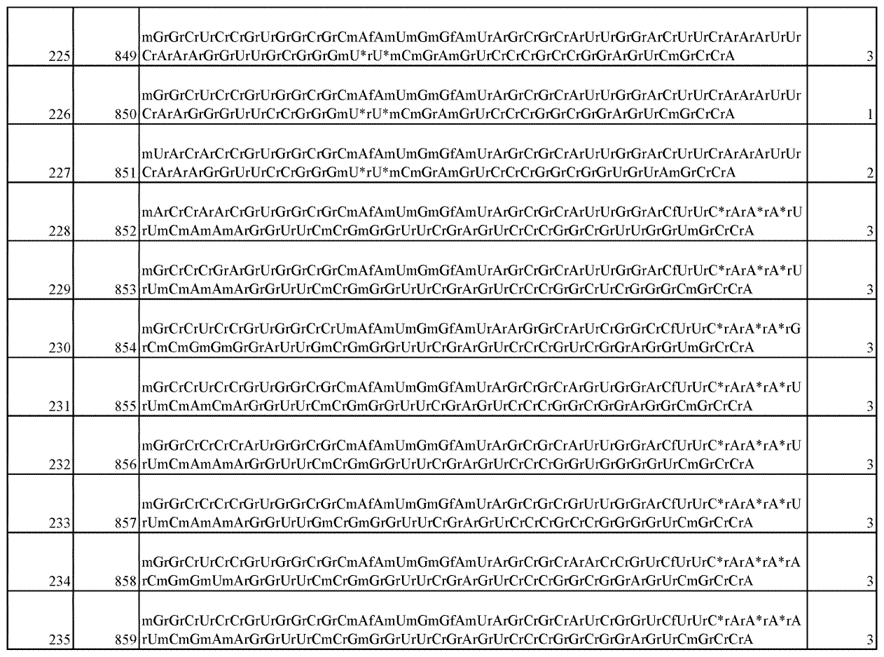

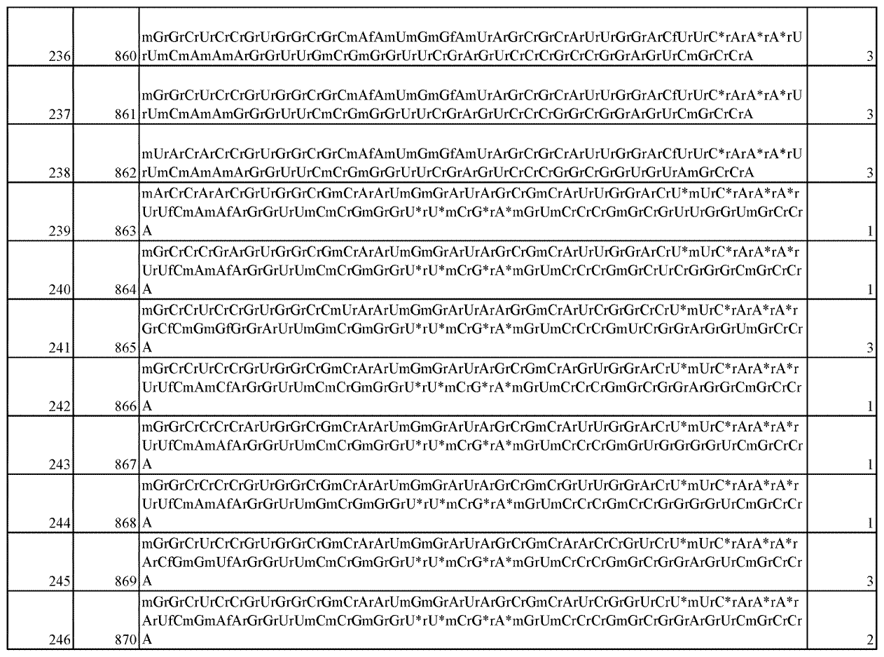

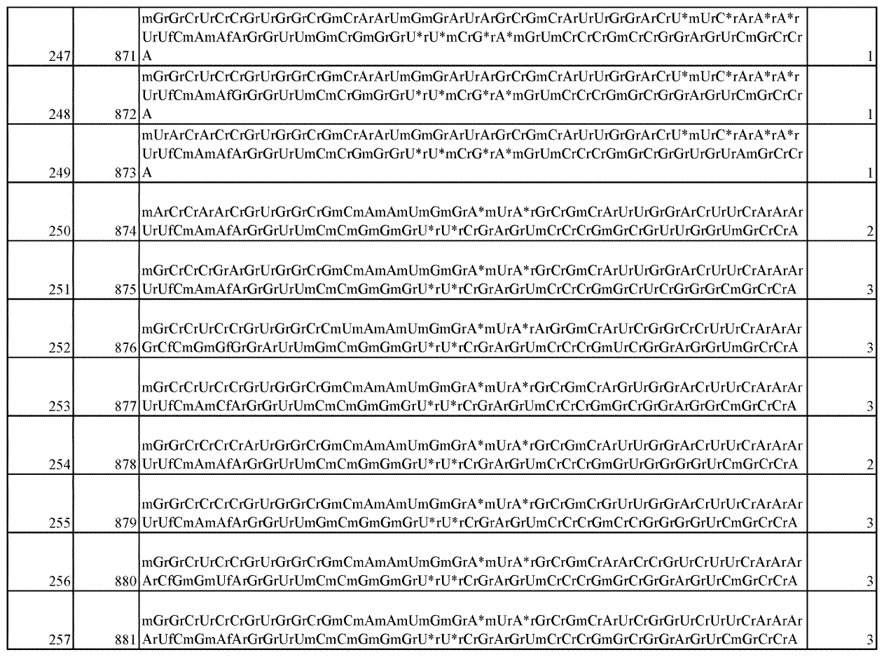

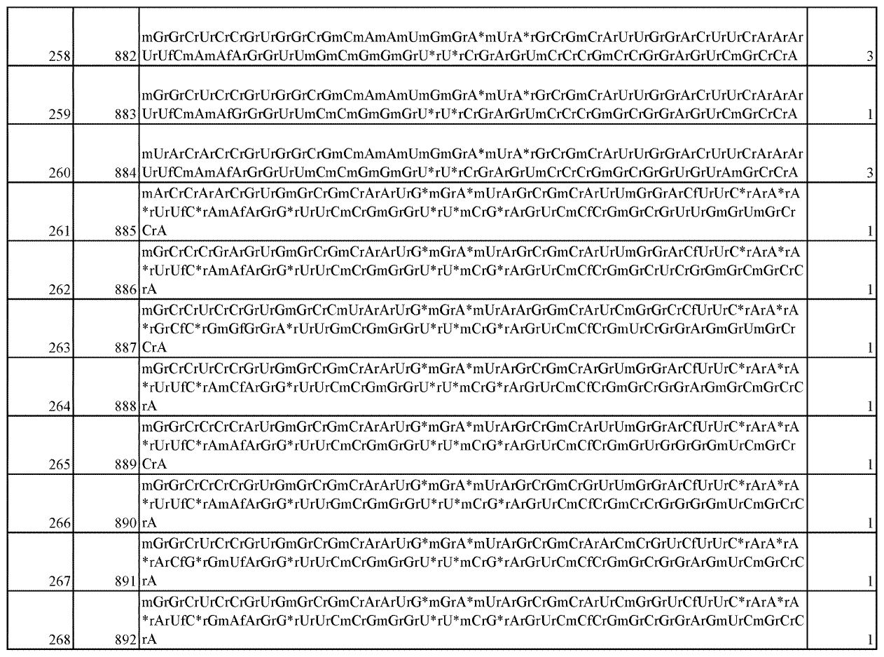

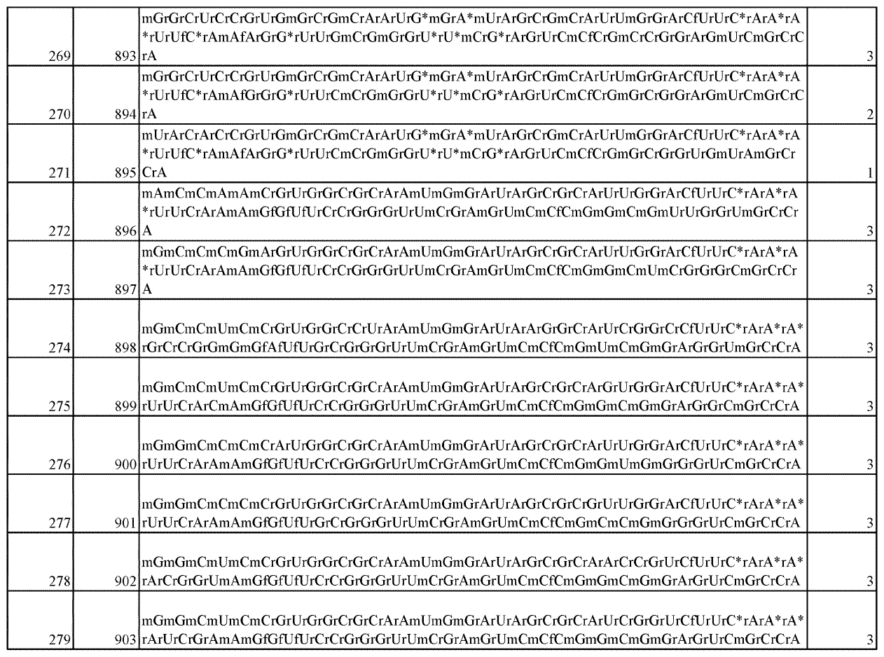

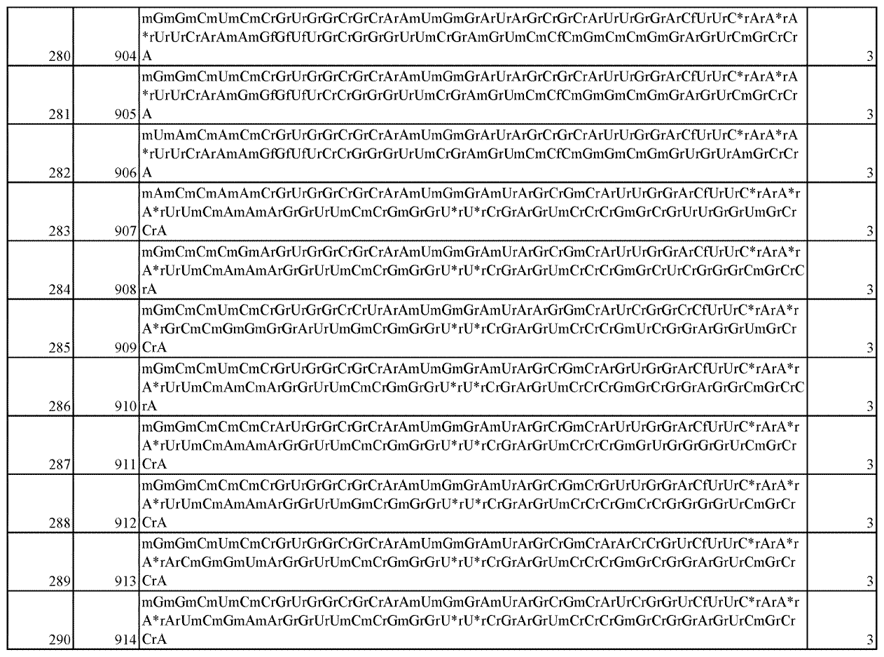

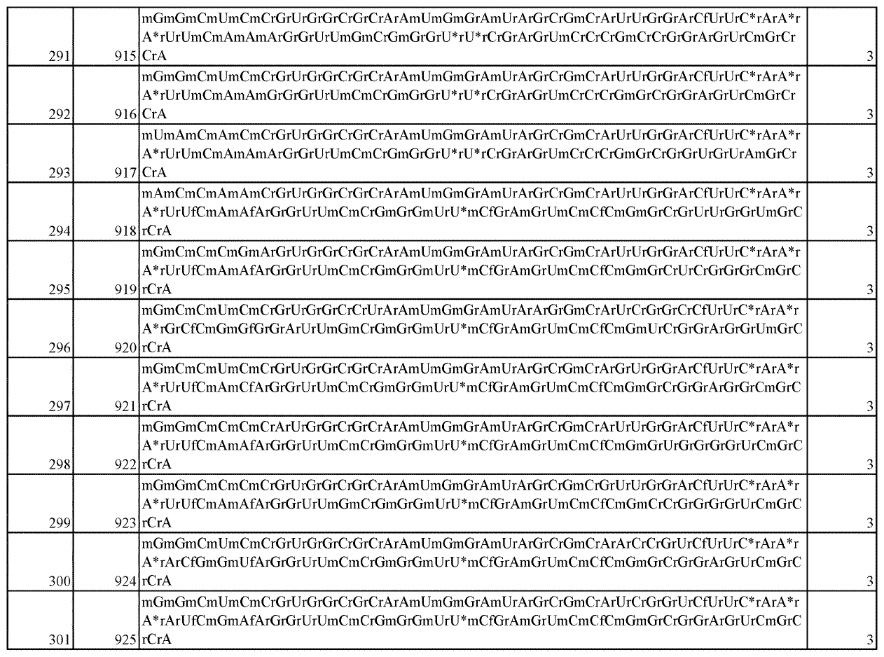

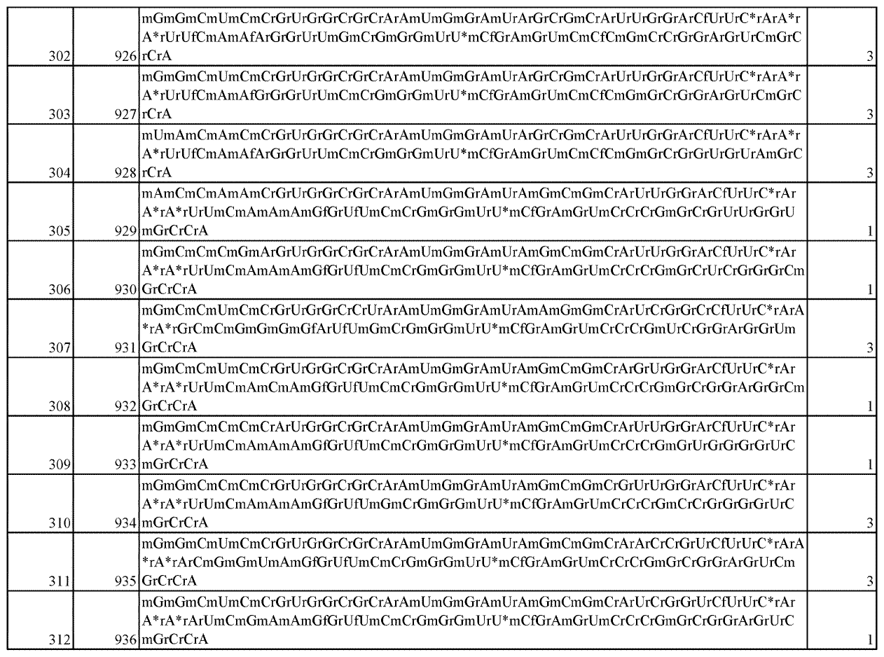

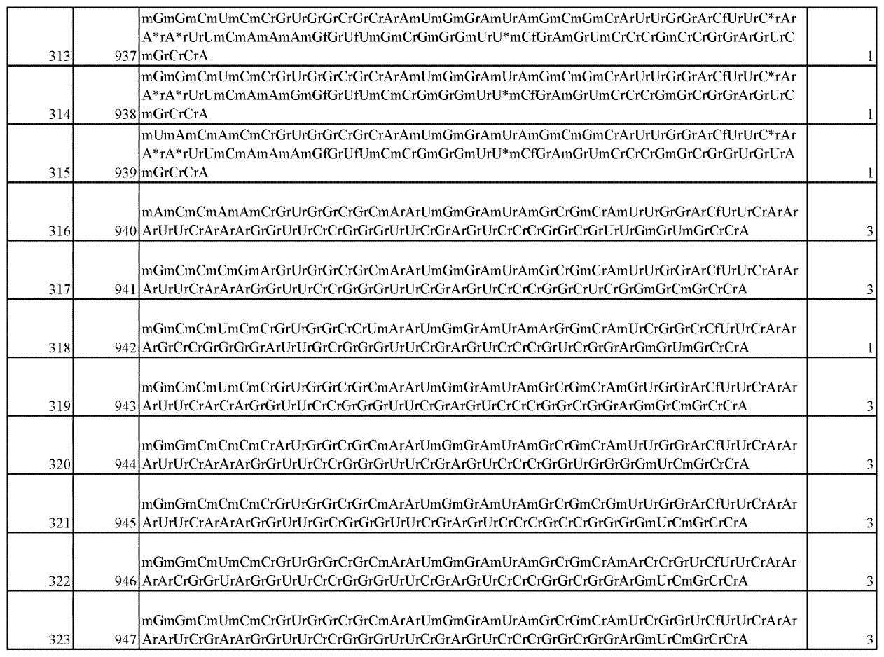

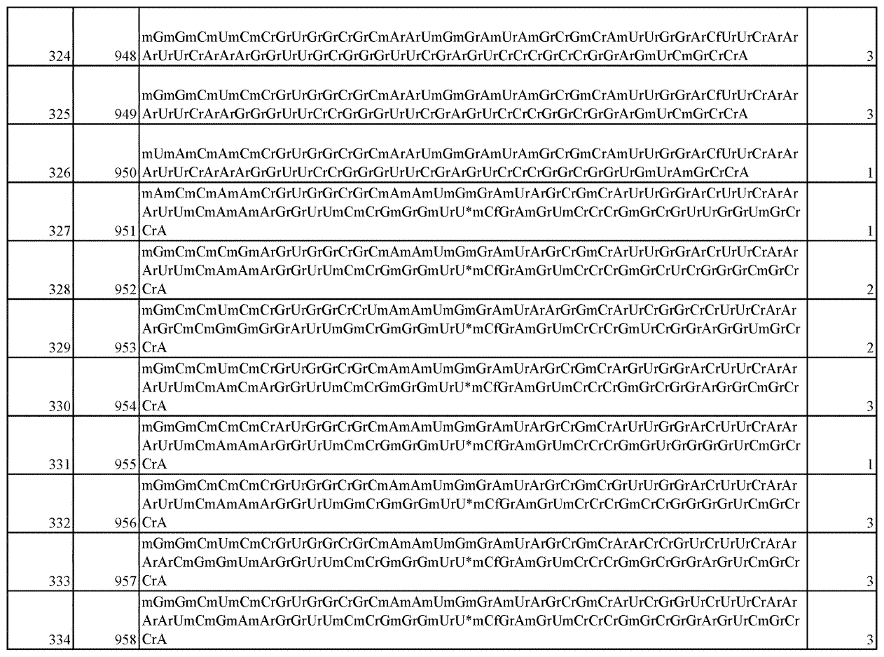

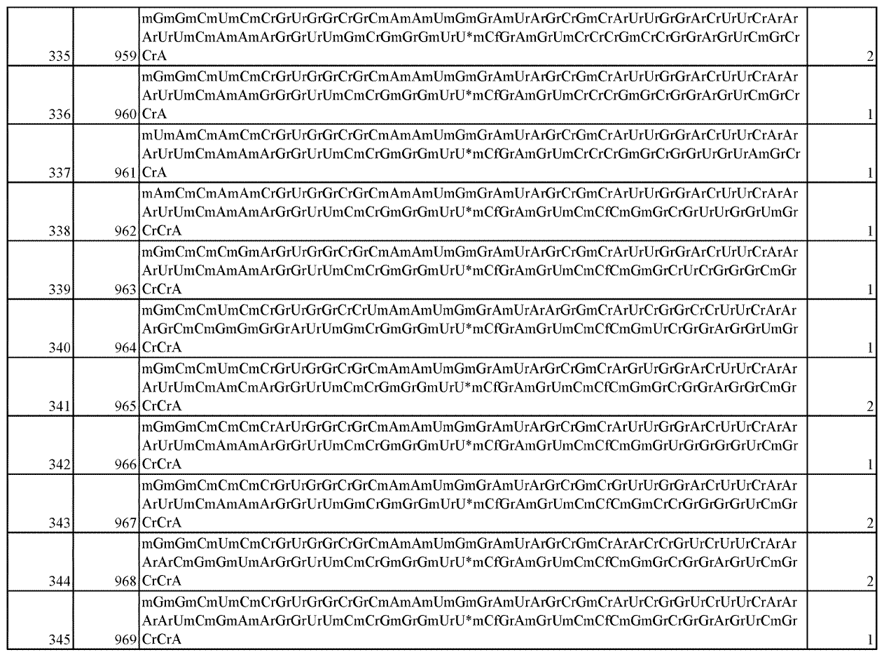

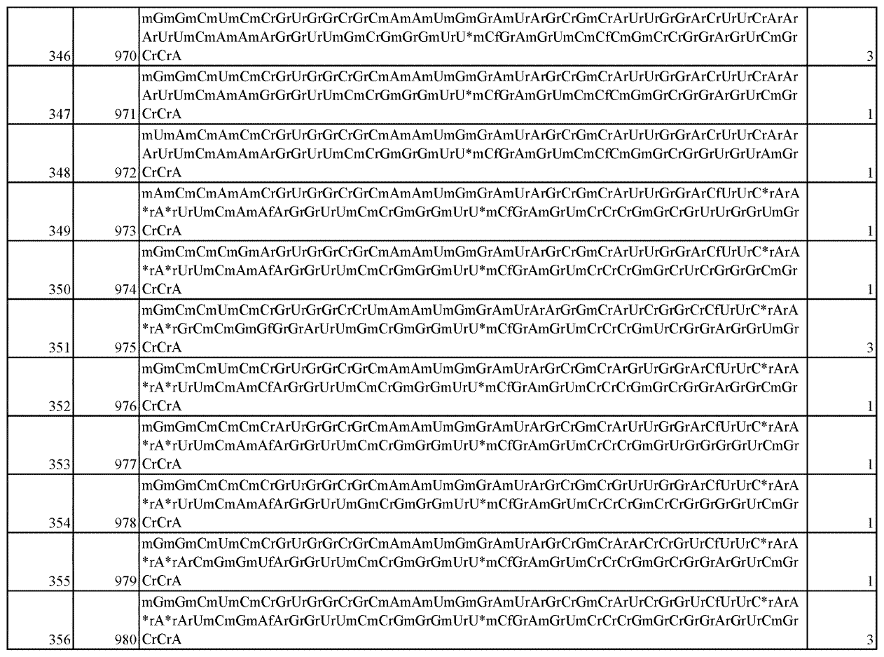

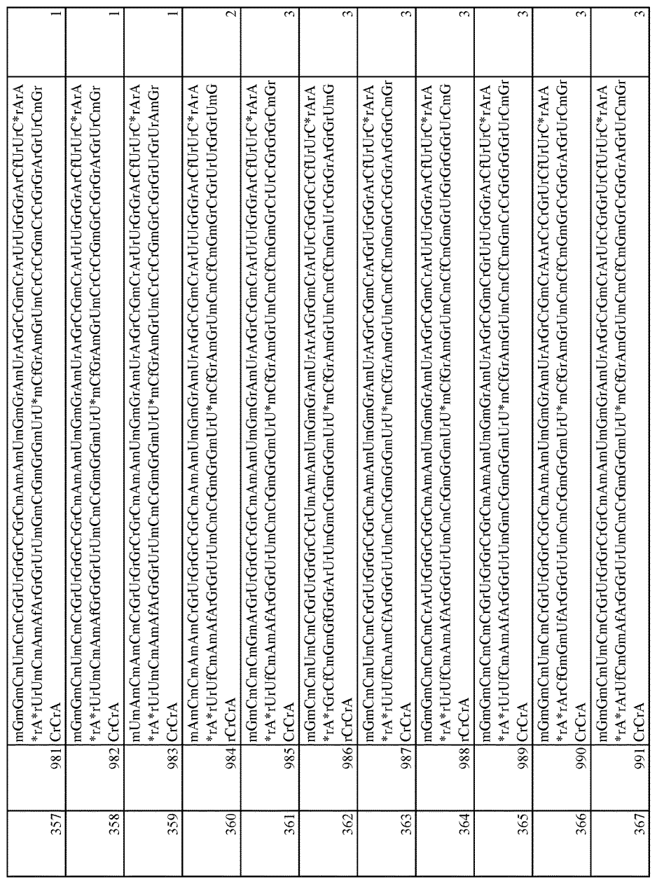

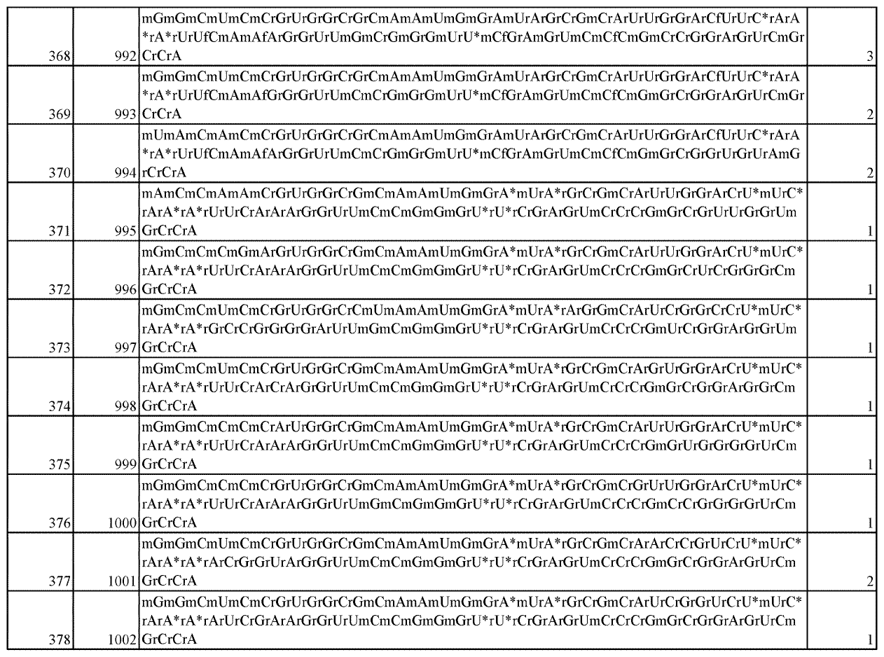

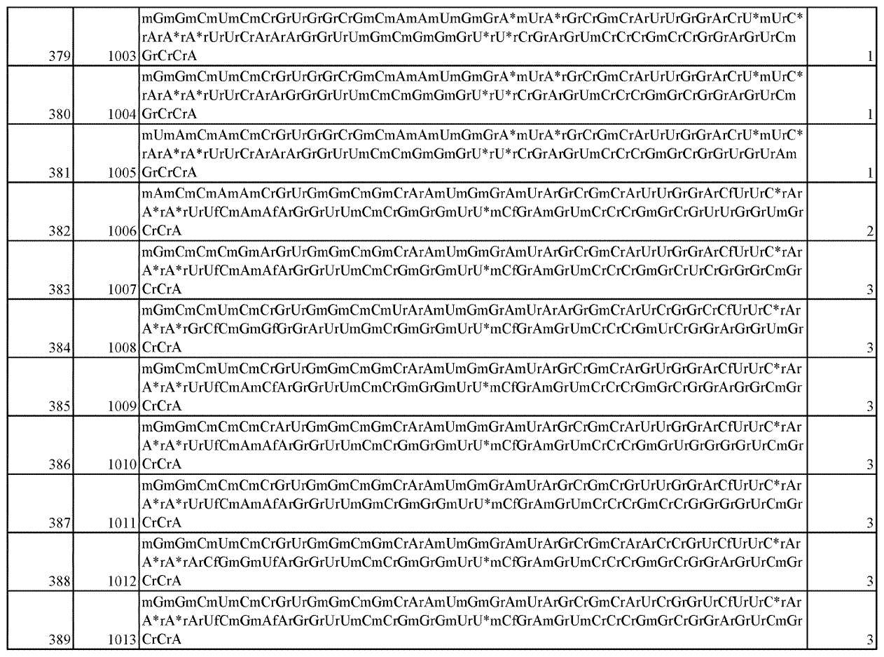

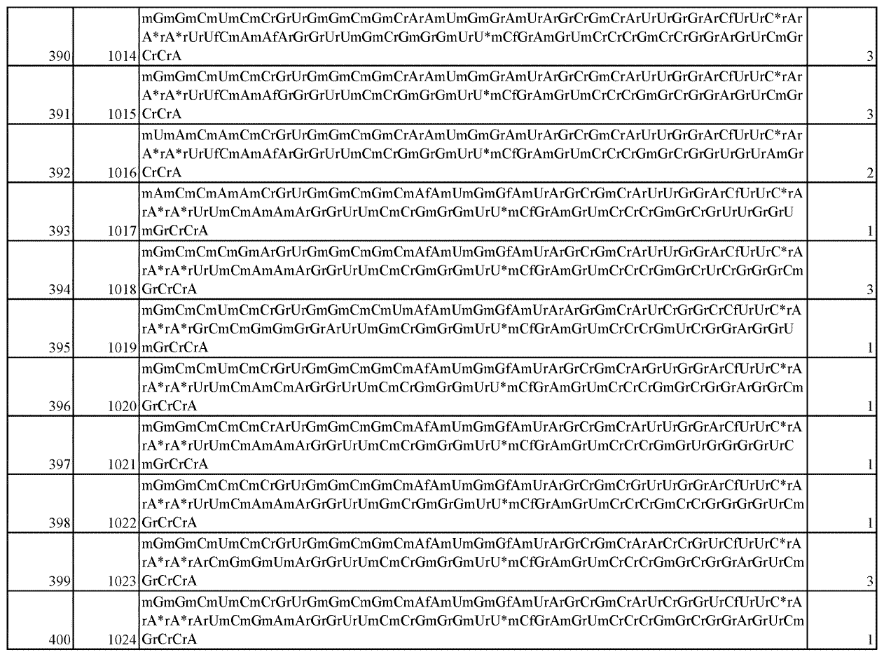

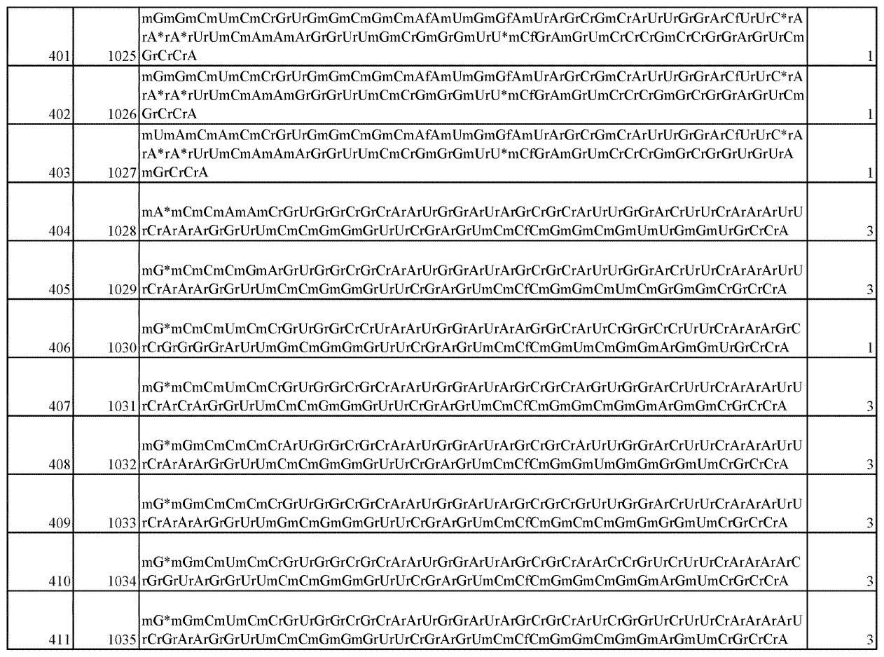

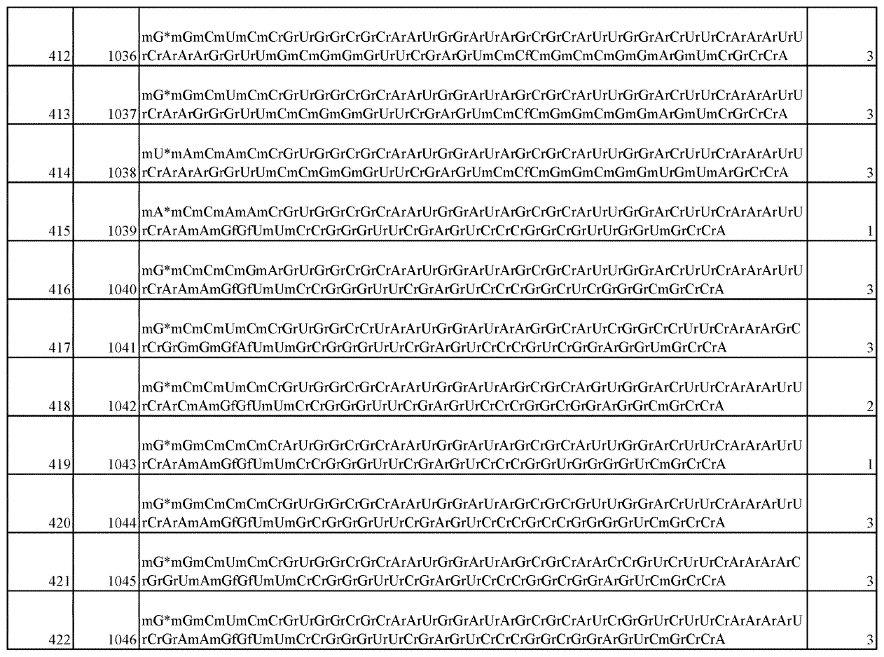

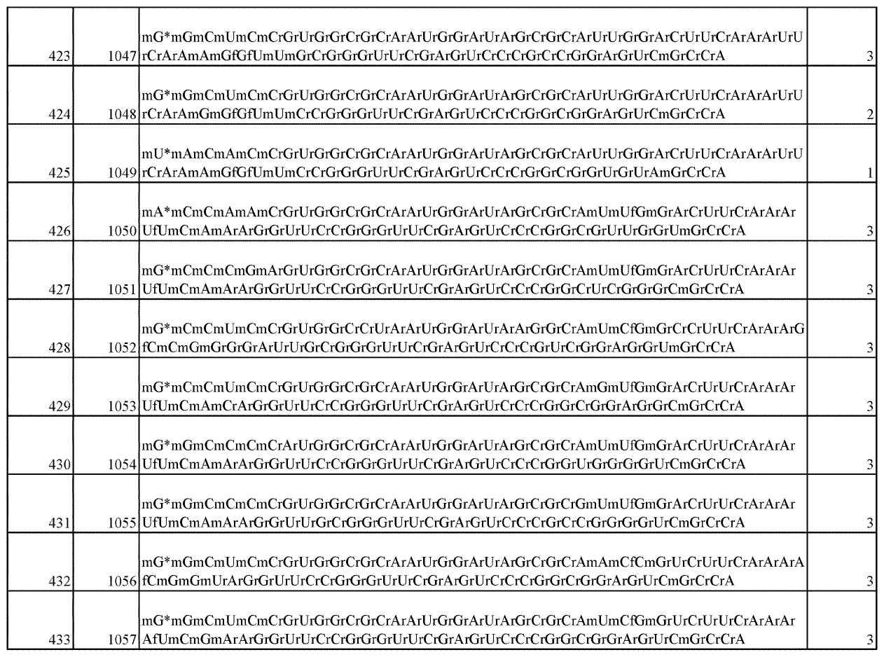

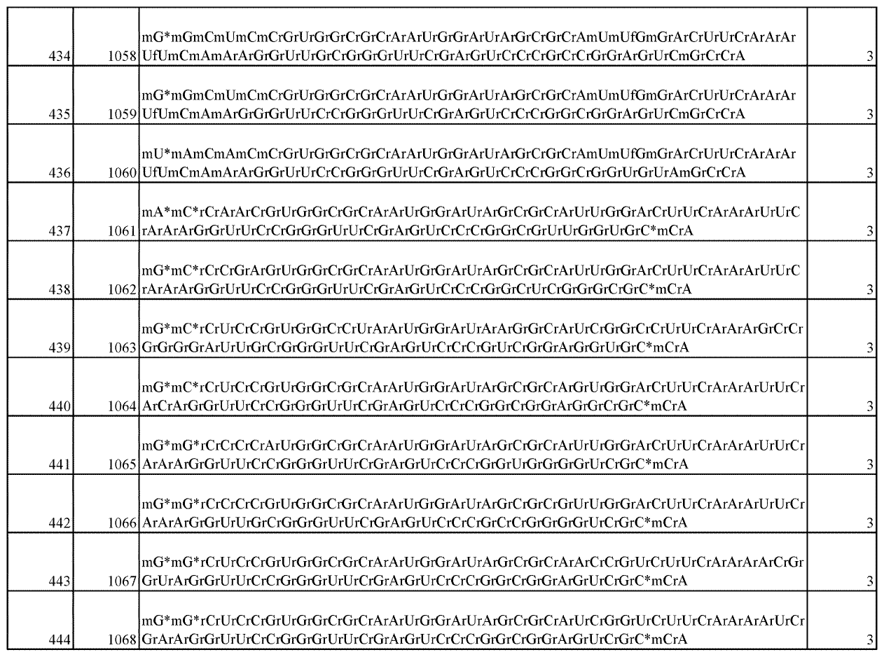

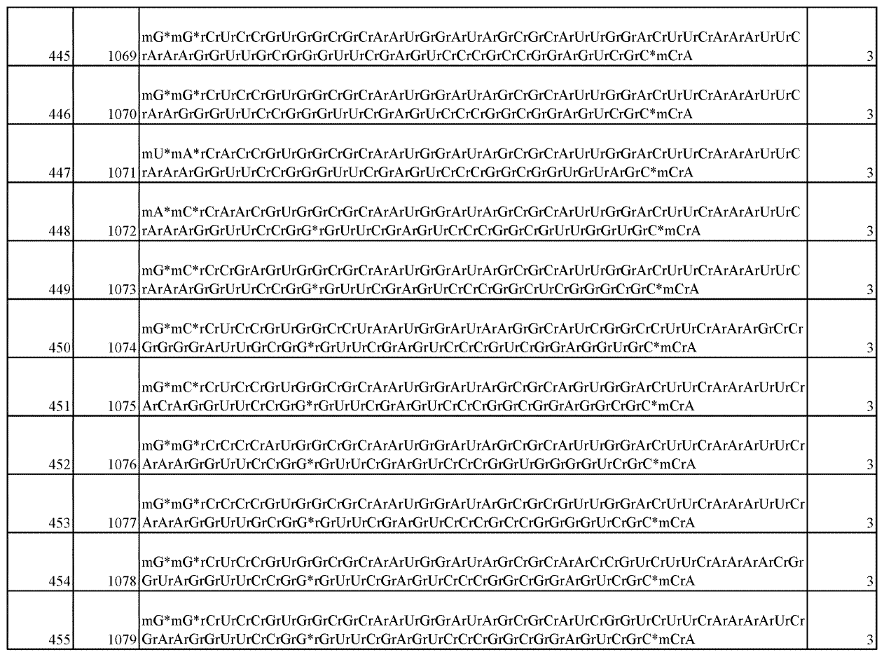

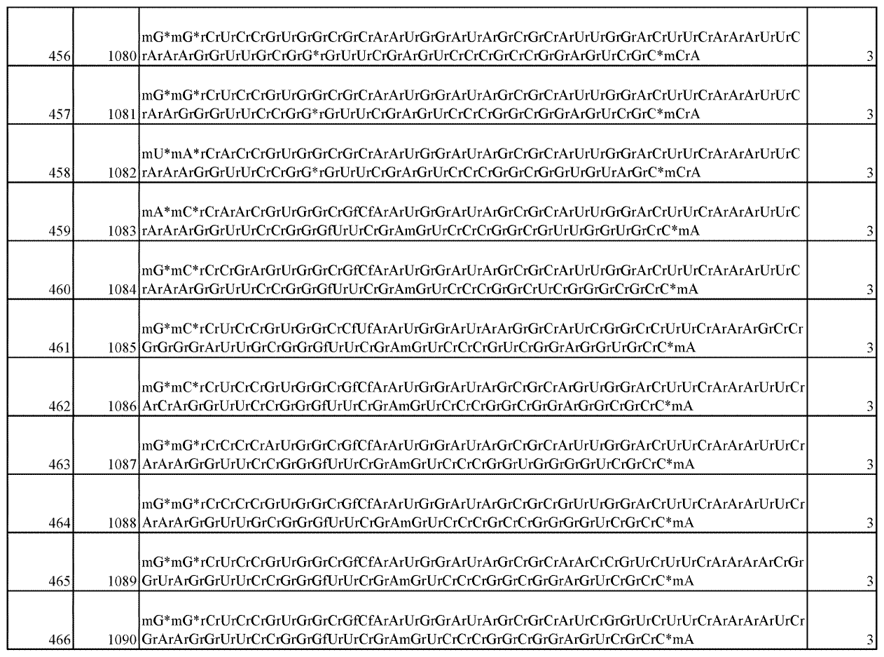

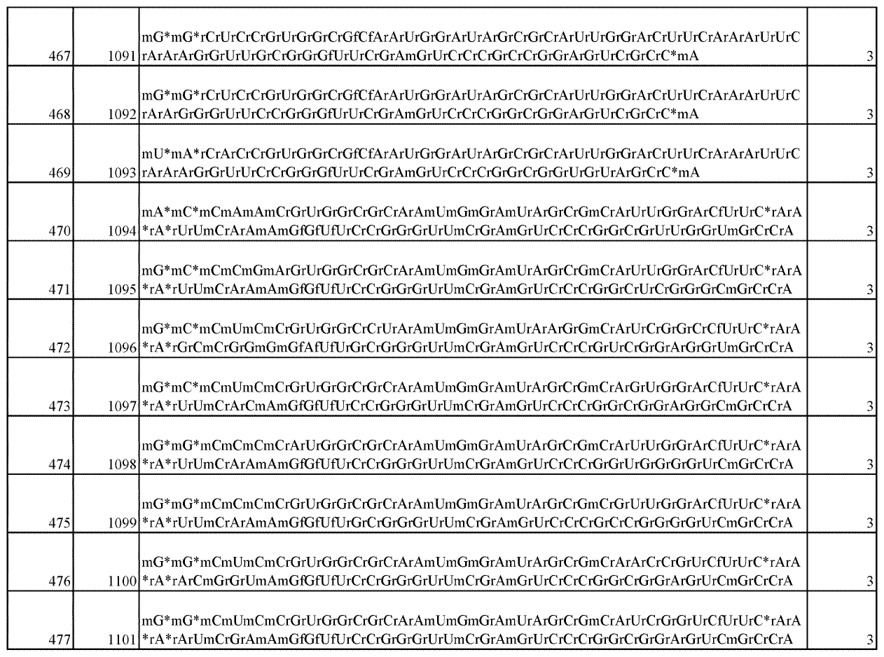

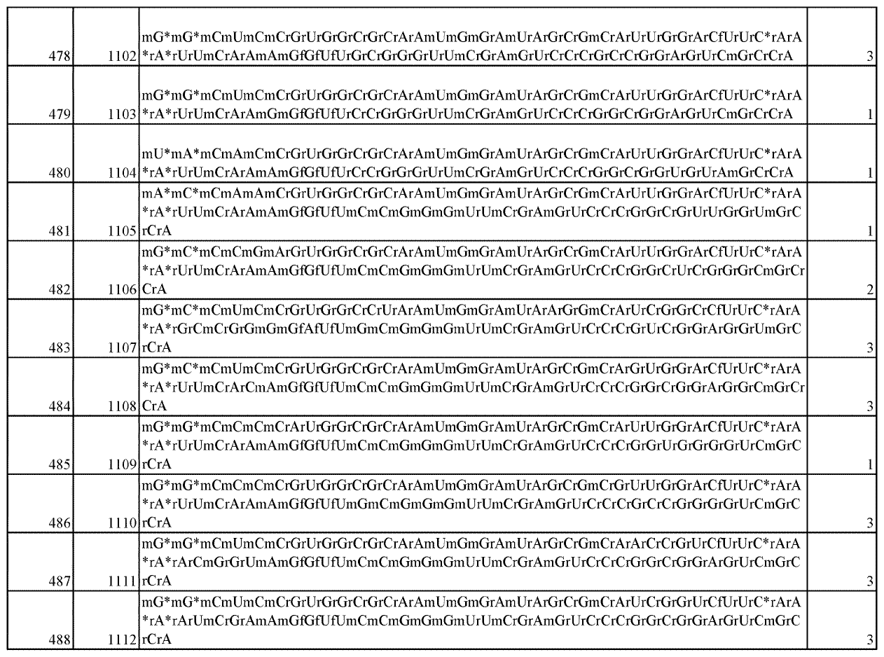

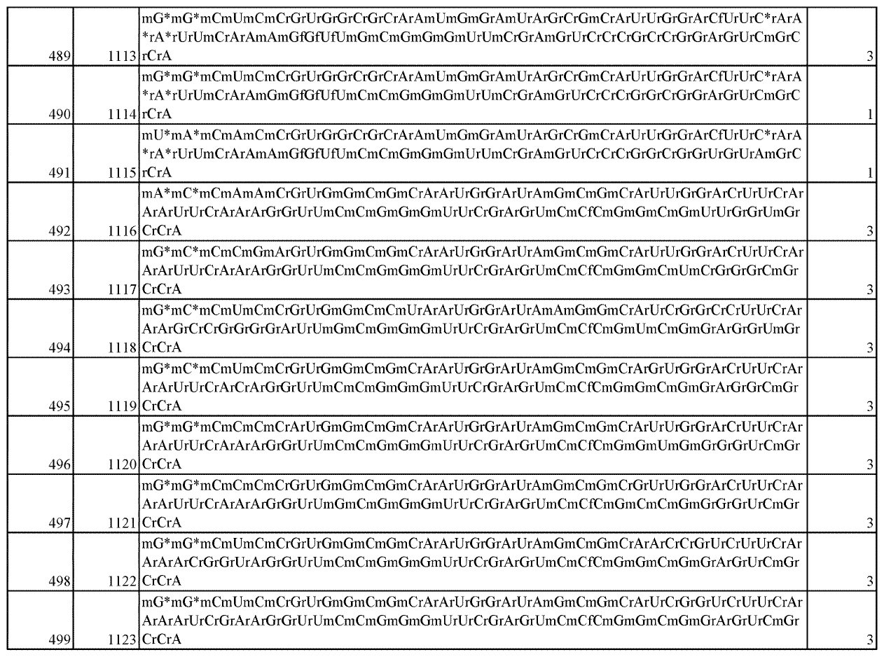

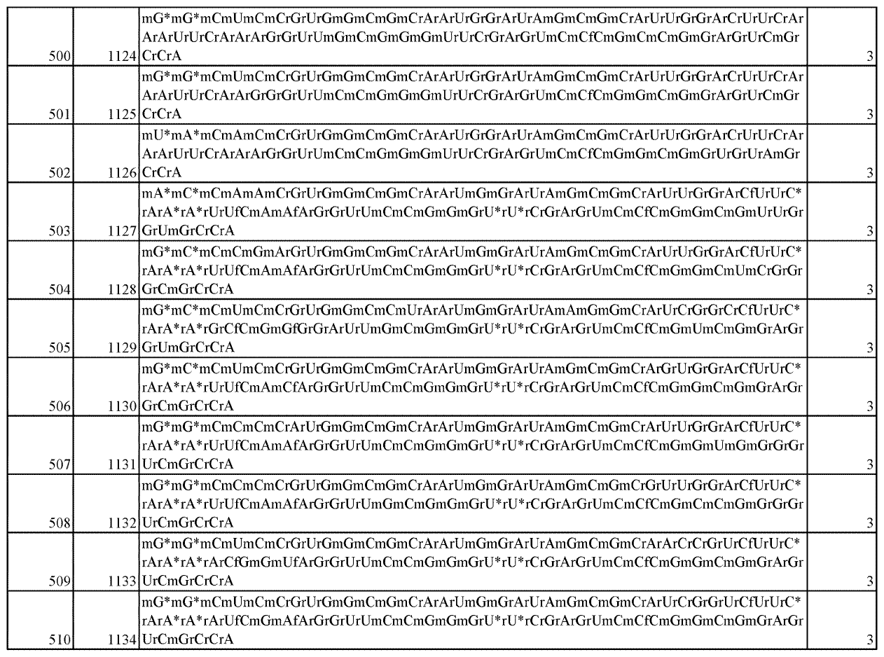

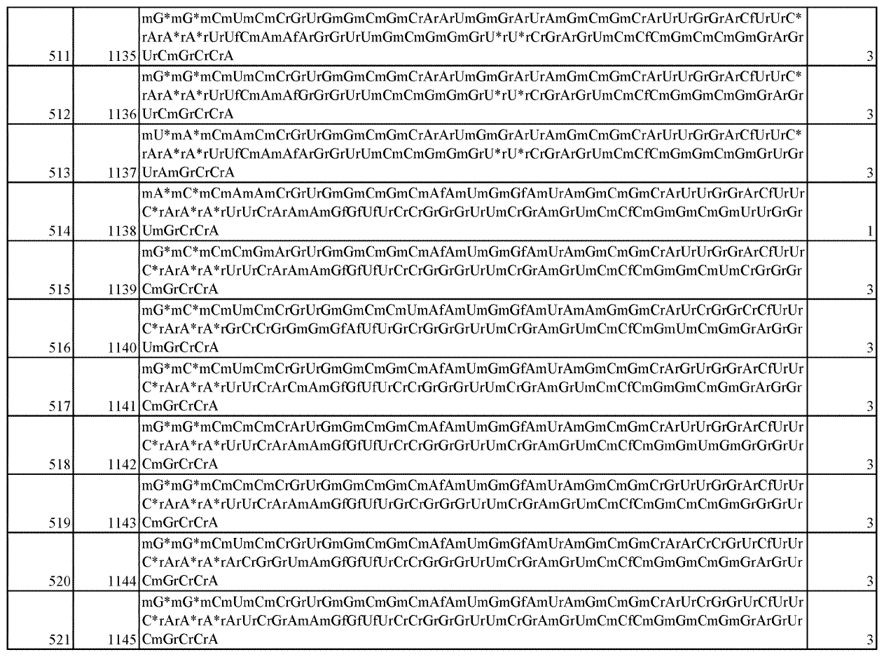

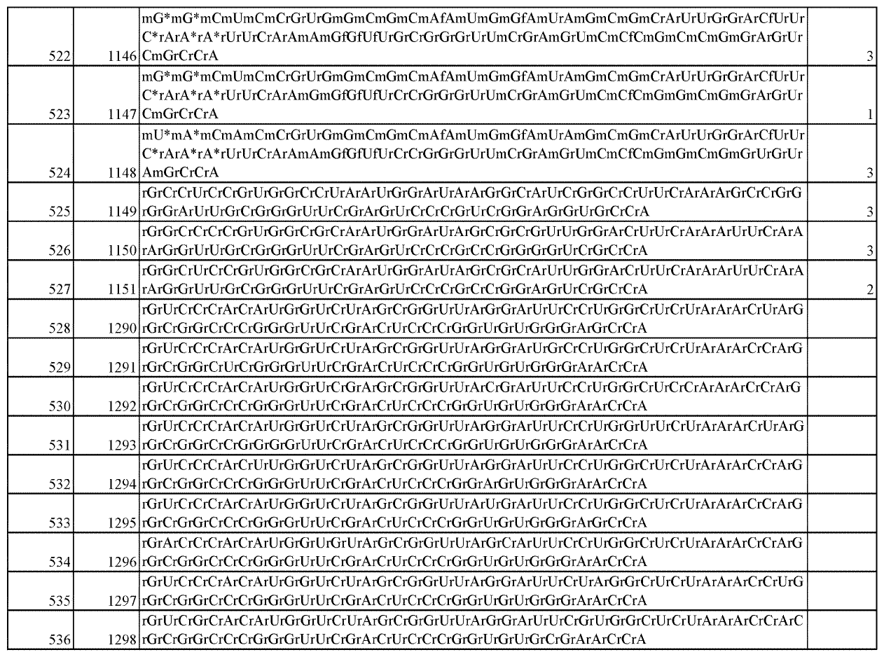

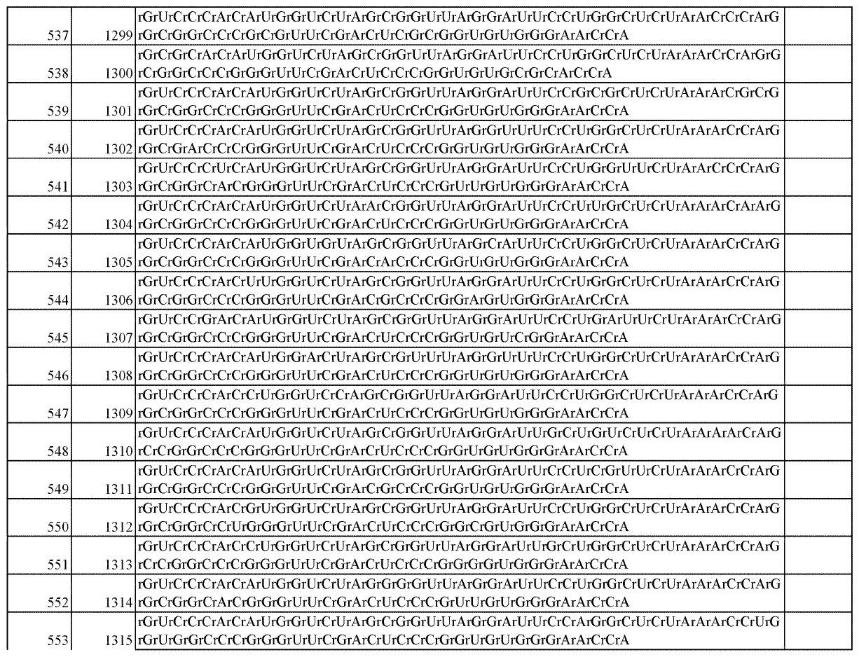

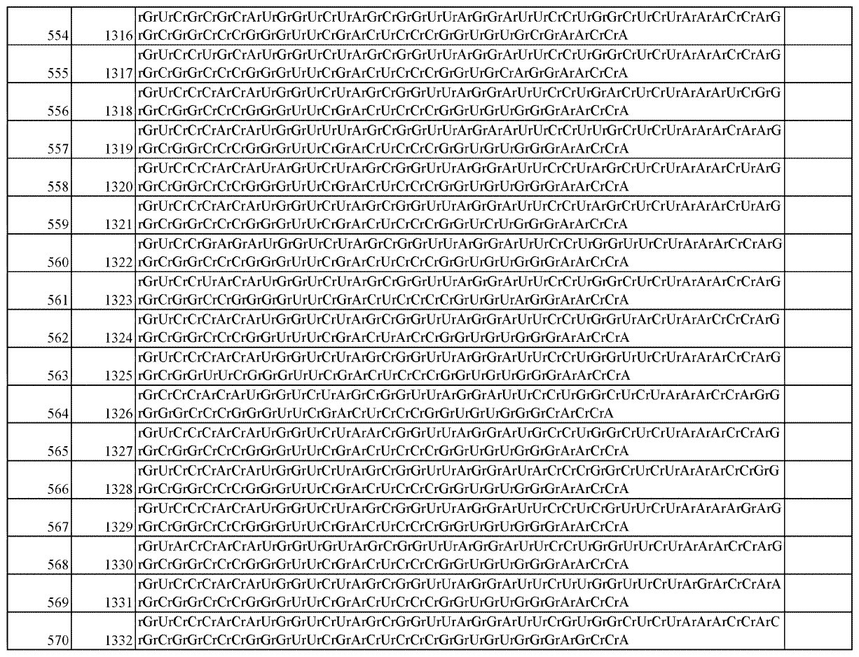

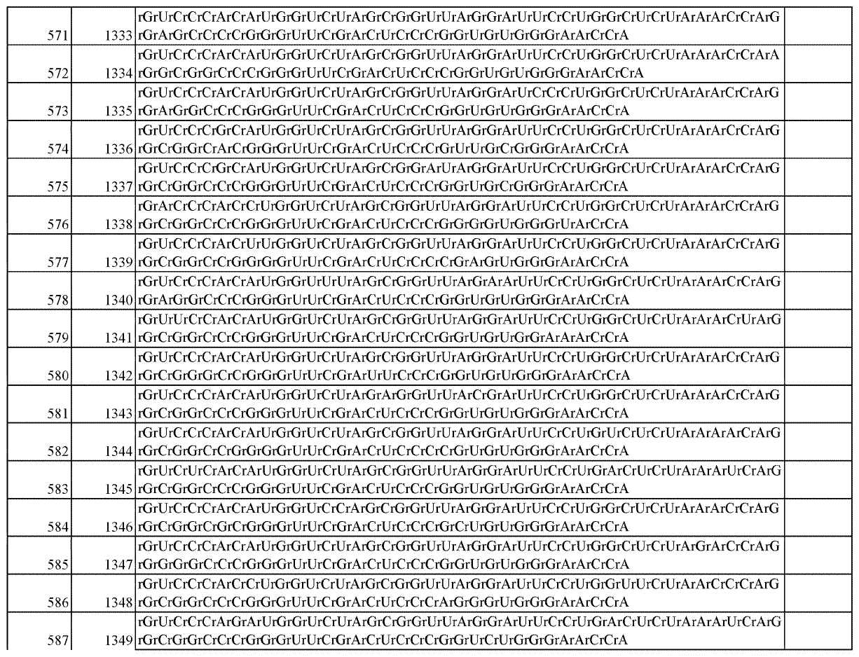

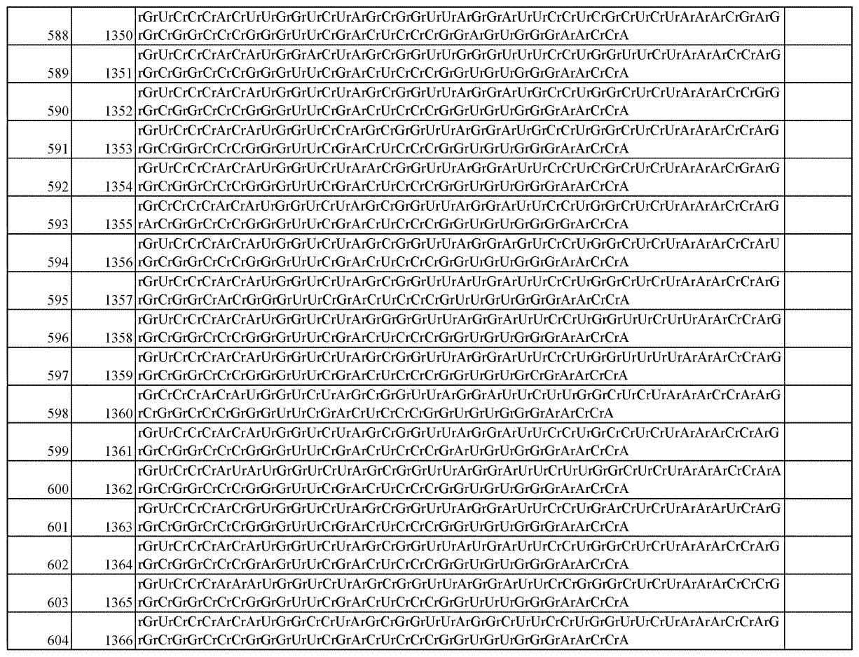

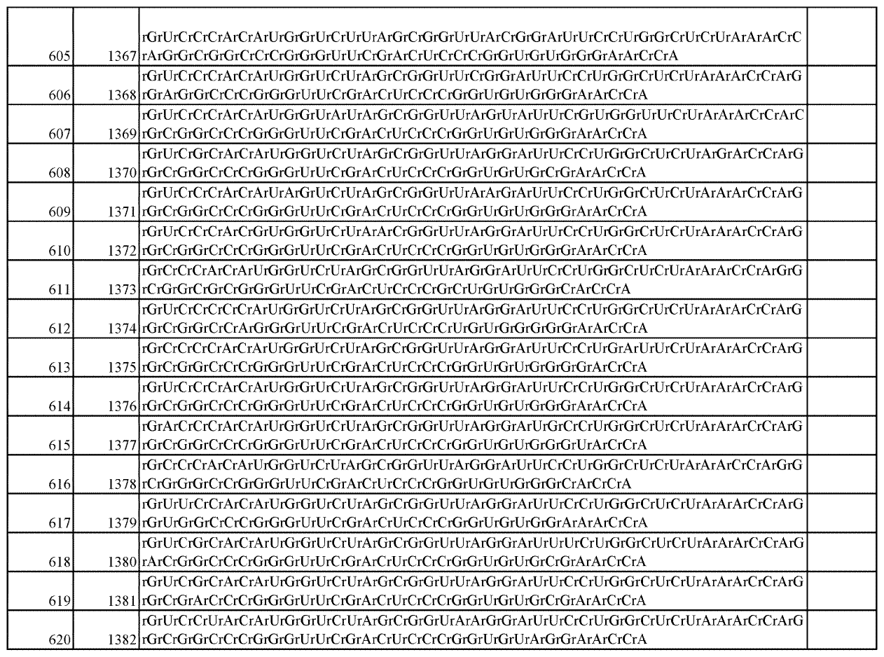

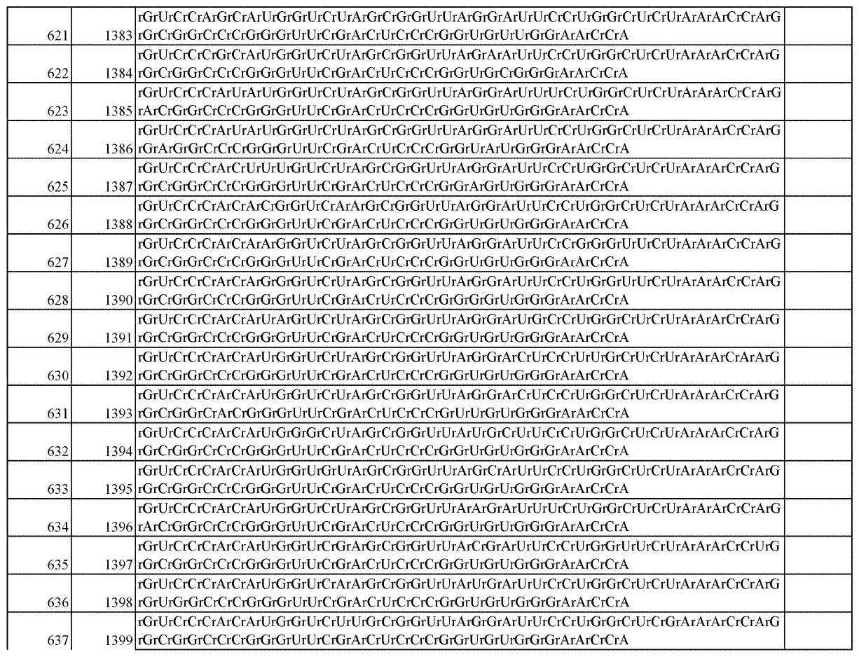

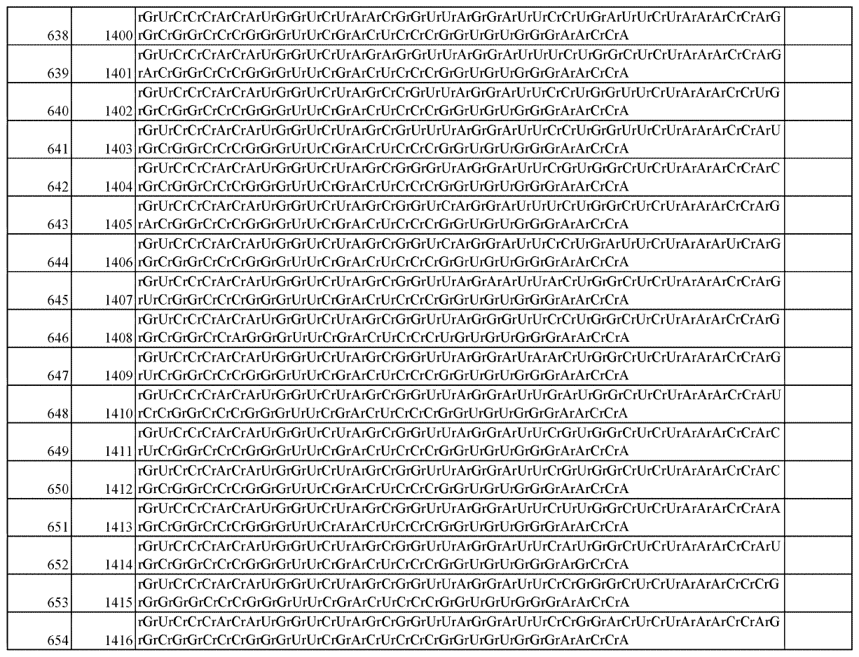

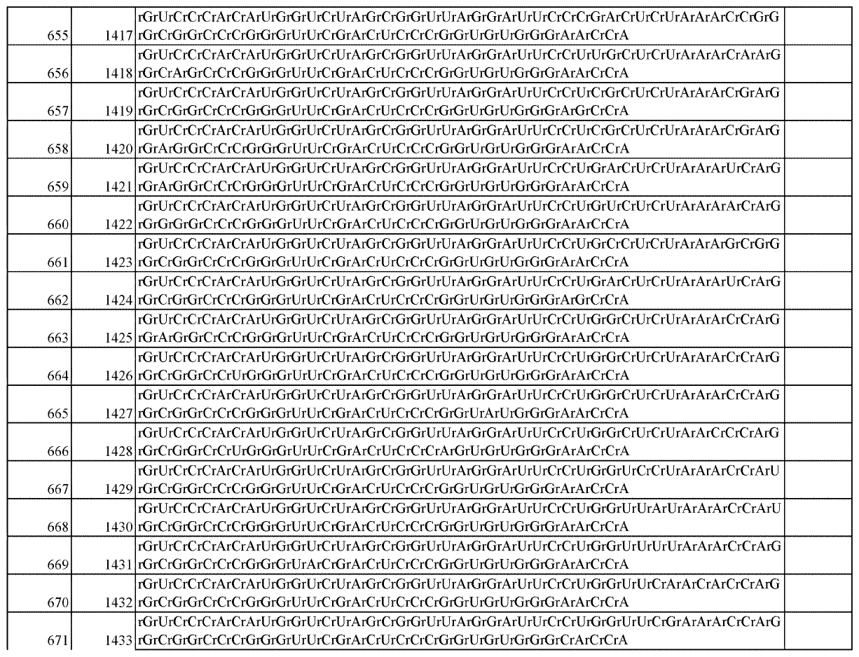

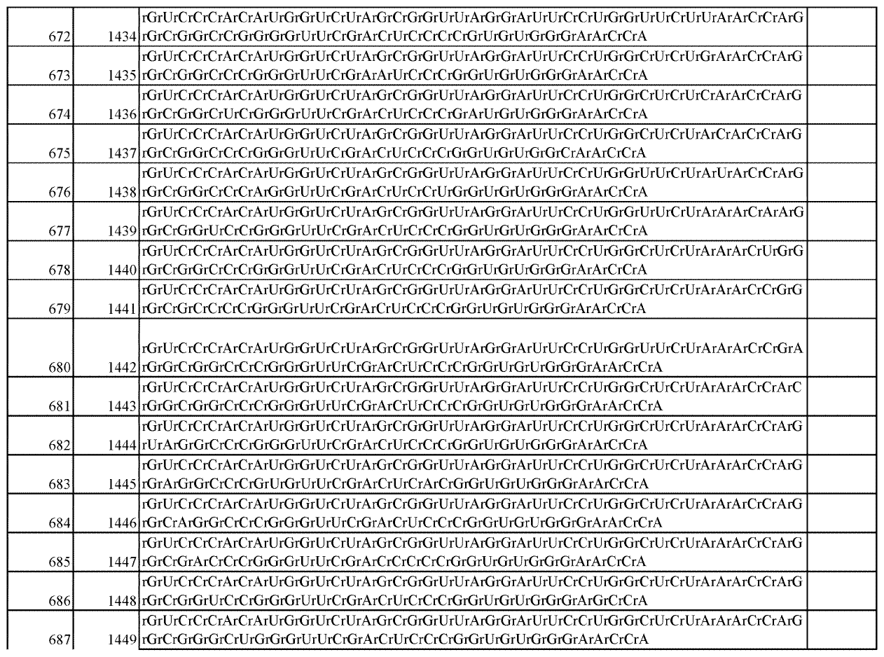

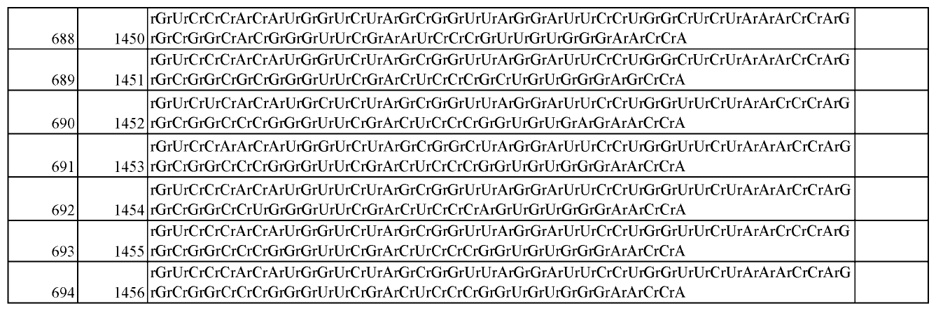

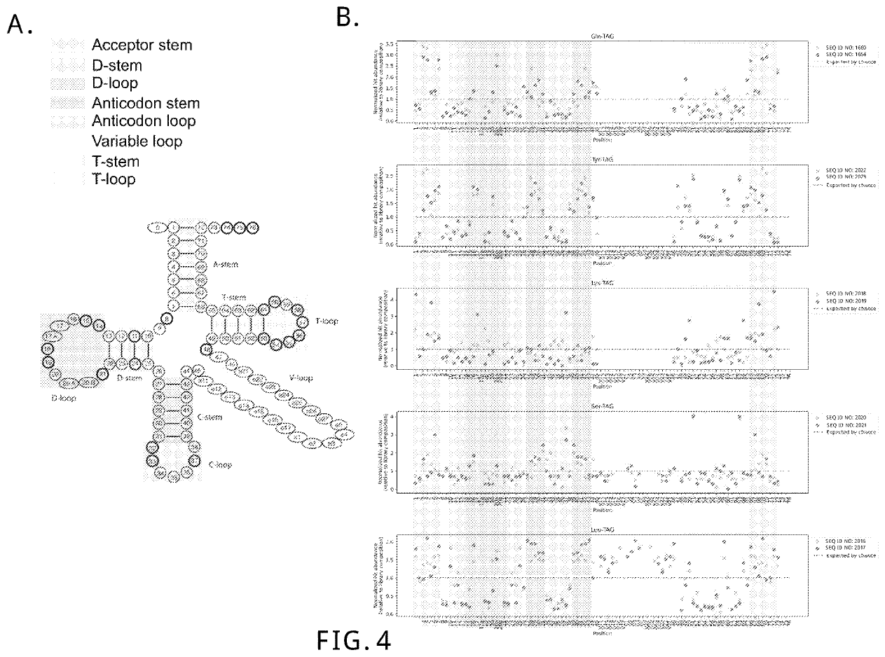

In one aspect, the present disclosure features a TREM comprising a nucleotide substitution at a nucleotide within the TREM sequence. In an embodiment, the nucleotide substitution is within a domain of the TREM, e.g., the [ASt Domain1], the [DH Domain], the [ACH Domain], the [VL Domain], the [TH Domain], or the [ASt Domain2]. In an embodiment, the nucleotide substitution is within the [ASt Domain1]. In an embodiment, the nucleotide substitution is within the [DH Domain]. In an embodiment, the nucleotide substitution is within the [ACH Domain]. In an embodiment, the nucleotide substitution is within the [VL Domain]. In an embodiment, the nucleotide substitution is within the [TH Domain]. In an embodiment, the nucleotide substitution is within the [ASt Domain2]. In an embodiment, the nucleotide substitution is at any of positions 1, 2, 3, 4, 5, 6, 7, 8, or 9 within the [ASt Domain1]. In an embodiment, the nucleotide substitution is at any of positions 10, 11, 12, 13, 14, 15, 16, 17, 18, 19, 20, 21, 22, 23, 24, 25, or 26 within the [DH Domain]. In an embodiment, the nucleotide substitution is at any of positions 27, 28, 29, 30, 31, 32, 33, 34, 35, 36, 37, 38, 39, 40, 41, 42, or 43 within the [ACH Domain]. In an embodiment, the nucleotide substitution is at any of positions 44, 45, 46, 47, 48, or 49 within the [VL Domain]. In an embodiment, the nucleotide substitution is at any of positions 50, 51, 52, 53, 54, 55, 56, 57, 58, 59, 60, 61, 62, 63, or 64 within the [TH Domain]. In an embodiment, the nucleotide substitution is at any of positions 65, 66, 67, 68, 69, 70, 71, 72, 73, 74, 75, or 76 within the [ASt Domain2]. In an embodiment, a nucleotide substitution is at a position within the TREM sequence, e.g., nucleotide position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 within the TREM sequence. In an embodiment, the nucleotide substitution is at position 1 within the TREM sequence. In an embodiment, the nucleotide substitution is at position 2 within the TREM sequence. In an embodiment, the nucleotide substitution is at position 3 within the TREM sequence. In an embodiment, the nucleotide substitution is at position 4 within the TREM sequence. In an embodiment, the nucleotide substitution is at position 5 within the TREM sequence. In an embodiment, the nucleotide substitution is at position 6 within the TREM sequence. In an embodiment, the nucleotide substitution is at position 7 within the TREM sequence. In an embodiment, the nucleotide substitution is at position 8 within the TREM sequence. In an embodiment, the nucleotide substitution is at position 9 within the TREM sequence. In an embodiment, the nucleotide substitution is at position 10 within the TREM sequence. In an embodiment, the nucleotide substitution is at position 11 within the TREM sequence. In an embodiment, the nucleotide substitution is at position 12 within the TREM sequence. In an embodiment, the nucleotide substitution is at position 13 within the TREM sequence. In an embodiment, the nucleotide substitution is at position 14 within the TREM sequence. In an embodiment, the nucleotide substitution is at position 15 within the TREM sequence. In an embodiment, the nucleotide substitution is at position 16 within the TREM sequence. In an embodiment, the nucleotide substitution is at position 17 within the TREM sequence. In an embodiment, the nucleotide substitution is at position 18 within the TREM sequence. In an embodiment, the nucleotide substitution is at position 19 within the TREM sequence. In an embodiment, the nucleotide substitution is at position 20 within the TREM sequence. In an embodiment, the nucleotide substitution is at position 21 within the TREM sequence. In an embodiment, the nucleotide substitution is at position 22 within the TREM sequence. In an embodiment, the nucleotide substitution is at position 23 within the TREM sequence. In an embodiment, the nucleotide substitution is at position 24 within the TREM sequence. In an embodiment, the nucleotide substitution is at position 25 within the TREM sequence. In an embodiment, the nucleotide substitution is at position 26 within the TREM sequence. In an embodiment, the nucleotide substitution is at position 27 within the TREM sequence. In an embodiment, the nucleotide substitution is at position 28 within the TREM sequence. In an embodiment, the nucleotide substitution is at position 29 within the TREM sequence. In an embodiment, the nucleotide substitution is at position 30 within the TREM sequence. In an embodiment, the nucleotide substitution is at position 31 within the TREM sequence. In an embodiment, the nucleotide substitution is at position 32 within the TREM sequence. In an embodiment, the nucleotide substitution is at position 33 within the TREM sequence. In an embodiment, the nucleotide substitution is at position 34 within the TREM sequence. In an embodiment, the nucleotide substitution is at position 35 within the TREM sequence. In an embodiment, the nucleotide substitution is at position 36 within the TREM sequence. In an embodiment, the nucleotide substitution is at position 37 within the TREM sequence. In an embodiment, the nucleotide substitution is at position 38 within the TREM sequence. In an embodiment, the nucleotide substitution is at position 39 within the TREM sequence. In an embodiment, the nucleotide substitution is at position 40 within the TREM sequence. In an embodiment, the nucleotide substitution is at position 41 within the TREM sequence. In an embodiment, the nucleotide substitution is at position 42 within the TREM sequence. In an embodiment, the nucleotide substitution is at position 43 within the TREM sequence. In an embodiment, the nucleotide substitution is at position 44 within the TREM sequence. In an embodiment, the nucleotide substitution is at position 45 within the TREM sequence. In an embodiment, the nucleotide substitution is at position 46 within the TREM sequence. In an embodiment, the nucleotide substitution is at position 47 within the TREM sequence. In an embodiment, the nucleotide substitution is at position 48 within the TREM sequence. In an embodiment, the nucleotide substitution is at position 49 within the TREM sequence. In an embodiment, the nucleotide substitution is at position 50 within the TREM sequence. In an embodiment, the nucleotide substitution is at position 51 within the TREM sequence. In an embodiment, the nucleotide substitution is at position 52 within the TREM sequence. In an embodiment, the nucleotide substitution is at position 53 within the TREM sequence. In an embodiment, the nucleotide substitution is at position 54 within the TREM sequence. In an embodiment, the nucleotide substitution is at position 55 within the TREM sequence. In an embodiment, the nucleotide substitution is at position 56 within the TREM sequence. In an embodiment, the nucleotide substitution is at position 57 within the TREM sequence. In an embodiment, the nucleotide substitution is at position 58 within the TREM sequence. In an embodiment, the nucleotide substitution is at position 59 within the TREM sequence. In an embodiment, the nucleotide substitution is at position 60 within the TREM sequence. In an embodiment, the nucleotide substitution is at position 61 within the TREM sequence. In an embodiment, the nucleotide substitution is at position 62 within the TREM sequence. In an embodiment, the nucleotide substitution is at position 63 within the TREM sequence. In an embodiment, the nucleotide substitution is at position 64 within the TREM sequence. In an embodiment, the nucleotide substitution is at position 65 within the TREM sequence. In an embodiment, the nucleotide substitution is at position 66 within the TREM sequence. In an embodiment, the nucleotide substitution is at position 67 within the TREM sequence. In an embodiment, the nucleotide substitution is at position 68 within the TREM sequence. In an embodiment, the nucleotide substitution is at position 69 within the TREM sequence. In an embodiment, the nucleotide substitution is at position 70 within the TREM sequence. In an embodiment, the nucleotide substitution is at position 71 within the TREM sequence. In an embodiment, the nucleotide substitution is at position 72 within the TREM sequence. In an embodiment, the nucleotide substitution is at position 73 within the TREM sequence. In an embodiment, the nucleotide substitution is at position 74 within the TREM sequence. In an embodiment, the nucleotide substitution is at position 75 within the TREM sequence. In an embodiment, the nucleotide substitution is at position 76 within the TREM sequence. In an embodiment, the nucleotide substitution is at position 77 within the TREM sequence. In an embodiment, the nucleotide substitution is at position 78 within the TREM sequence. In an embodiment, the nucleotide substitution is at position 79 within the TREM sequence. In an embodiment, the nucleotide substitution is at position 80 within the TREM sequence. In an embodiment, the nucleotide substitution is at position 81 within the TREM sequence. In an embodiment, the nucleotide substitution is at position 82 within the TREM sequence. In an embodiment, the nucleotide substitution is at position 83 within the TREM sequence. In an embodiment, the nucleotide substitution is at position 84 within the TREM sequence. In an embodiment, the nucleotide substitution is at position 85 within the TREM sequence. In an embodiment, the nucleotide substitution is at position 86 within the TREM sequence. In an embodiment, the nucleotide substitution is at position 87 within the TREM sequence. In an embodiment, the nucleotide substitution is at position 88 within the TREM sequence. In an embodiment, the nucleotide substitution is at position 89 within the TREM sequence. In an embodiment, the nucleotide substitution is at position 90 within the TREM sequence. In an embodiment, the nucleotide substitution is at position 91 within the TREM sequence. In an embodiment, the nucleotide substitution is at position 92 within the TREM sequence. In an embodiment, the nucleotide substitution is at position 93 within the TREM sequence. In an embodiment, the nucleotide substitution is at position 94 within the TREM sequence. In an embodiment, the nucleotide substitution is at position 95 within the TREM sequence. In an embodiment, the nucleotide substitution is at position 96 within the TREM sequence. In an embodiment, the nucleotide substitution is at position 97 within the TREM sequence. In an embodiment, the nucleotide substitution is at position 98 within the TREM sequence. In an embodiment, the nucleotide substitution is at position 99 within the TREM sequence. In an embodiment, the nucleotide substitution is at position 100 within the TREM sequence. In an embodiment, a TREM comprises multiple nucleotide substitutions, e.g., 1, 2, 3, 4, 5, 6, 7, 8, 9, 10, 11, 12, 13, 14, 15, 16, 17, 18, 19, 20 or more nucleotide substitutions. In an embodiment, a TREM comprises 1 nucleotide substitution. In an embodiment, a TREM comprises 2 nucleotide substitutions. In an embodiment, a TREM comprises 3 nucleotide substitutions. In an embodiment, a TREM comprises 4 nucleotide substitutions. In an embodiment, a TREM comprises 5 nucleotide substitutions. In an embodiment, a TREM comprises 6 nucleotide substitutions. In an embodiment, a TREM comprises 7 nucleotide substitutions. In an embodiment, a TREM comprises 8 nucleotide substitutions. In an embodiment, a TREM comprises 9 nucleotide substitutions. In an embodiment, a TREM comprises 10 nucleotide substitutions. In an embodiment, a TREM comprises 11 nucleotide substitutions. In an embodiment, a TREM comprises 12 nucleotide substitutions. In an embodiment, a TREM comprises 13 nucleotide substitutions. In an embodiment, a TREM comprises 14 nucleotide substitutions. In an embodiment, a TREM comprises 15 nucleotide substitutions. In an embodiment, a TREM comprises 16 nucleotide substitutions. In an embodiment, a TREM comprises 17 nucleotide substitutions. In an embodiment, a TREM comprises 18 nucleotide substitutions. In an embodiment, a TREM comprises 19 nucleotide substitutions. In an embodiment, a TREM comprises 20 nucleotide substitutions. In an embodiment, a TREM comprises more than 20 nucleotide substitutions. In an embodiment, a TREM comprises nucleotide substitutions in more than one TREM domain, e.g., more than one of [ASt Domain1], [DH Domain], [ACH Domain], [VL Domain], [TH Domain], or [ASt Domain2]. In an embodiment, a TREM comprises nucleotide substitutions in one TREM domain. In an embodiment, a TREM comprises nucleotide substitutions in two TREM domains. In an embodiment, a TREM comprises nucleotide substitutions in three TREM domains. In an embodiment, a TREM comprises nucleotide substitutions in four TREM domains. In an embodiment, a TREM comprises nucleotide substitutions in five TREM domains. In an embodiment, a TREM comprises nucleotide substitutions in six TREM domains. In an embodiment, a TREM comprises nucleotide substitutions in the [ASt Domain1] and the [DH Domain]. In an embodiment, a TREM comprises nucleotide substitutions in the [ASt Domain1] and the [ACH Domain]. In an embodiment, a TREM comprises nucleotide substitutions in the [ASt Domain1] and the [VL Domain]. In an embodiment, a TREM comprises nucleotide substitutions in the [ASt Domain1] and the [TH Domain]. In an embodiment, a TREM comprises nucleotide substitutions in the [ASt Domain1] and the [ASt Domain2]. In an embodiment, a TREM comprises nucleotide substitutions in the [DH Domain] and the [ACH Domain]. In an embodiment, a TREM comprises nucleotide substitutions in the [DH Domain] and the [ACH Domain]. In an embodiment, a TREM comprises nucleotide substitutions in the [DH Domain] and the [VL Domain]. In an embodiment, a TREM comprises nucleotide substitutions in the [DH Domain] and the [TH Domain]. In an embodiment, a TREM comprises nucleotide substitutions in the [DH Domain] and the [ASt Domain2]. In an embodiment, a TREM comprises nucleotide substitutions in the [ACH Domain] and the [VL Domain]. In an embodiment, a TREM comprises nucleotide substitutions in the [ACH Domain] and the [TH Domain]. In an embodiment, a TREM comprises nucleotide substitutions in the [ACH Domain] and the [ASt Domain2]. In an embodiment, a TREM comprises nucleotide substitutions in the [VL Domain] and the [TH Domain]. In an embodiment, a TREM comprises nucleotide substitutions in the [VL Domain] and the [ASt Domain2]. In an embodiment, a TREM comprises nucleotide substitutions in the [TH Domain] and the [ASt Domain2]. In an embodiment, a TREM comprises nucleotide substitutions in the [ASt Domain1] and the [ASt Domain1]. In an embodiment, a TREM comprises nucleotide substitutions in the [DH Domain] and the [DH Domain]. In an embodiment, a TREM comprises nucleotide substitutions in the [ACH Domain] and the [ACH Domain]. In an embodiment, a TREM comprises nucleotide substitutions in the [VL Domain] and the [VL Domain]. In an embodiment, a TREM comprises nucleotide substitutions in the [TH Domain] and the [TH Domain]. In an embodiment, a TREM comprises nucleotide substitutions in the [ASt Domain2] and the [ASt Domain2]. In an embodiment, the nucleotide substitution is within a loop of the TREM secondary structure, e.g., an unpaired region of the TREM. In an embodiment, the nucleotide substitution is at any of unpaired positions 8, 9, 14, 15, 16, 17, 17A, 18, 19, 20, 20A, 20B, 21, 32, 33, 34, 35, 36, 37, 38, 45, e11, e12, e13, e14, e15, e16, e17, e1, e2, e3, e4, e5, e27, e26, e25, e24, e23, e22, e21, 46, 47, 48, 54, 55, 56, 57, 58, 59, 60, 73, 74, 75, or 76 according to the universal tRNA numbering scheme, e.g., the nucleotide position numbering as provided in FIG. 4. In an embodiment, the nucleotide substitution is within a stem of the TREM secondary structure, e.g., within a base-paired region of the TREM. In an embodiment, the nucleotide substitution is at any of base-paired positions 1, 2, 3, 4, 5, 6, 7, 10, 11, 12, 13, 22, 23, 24, 25, 27, 28, 29, 30, 31, 39, 40, 41, 42, 43, 49, 50, 51, 52, 53, 61, 62, 63, 64, 65, 66, 67, 68, 69, 70, 71, or 72 according to the universal tRNA numbering scheme, e.g., the nucleotide position numbering as provided in FIG. 4. In an embodiment, a nucleotide substitution maintains the secondary structure, e.g., base-pairing, of the TREM. In an embodiment, a nucleotide substitution does not maintain the secondary structure, e.g., base-pairing, of the TREM. In an embodiment, a TREM comprising a first nucleotide substitution at a base-paired position further comprises a second compensatory nucleotide substitution, e.g., a nucleotide substitution at the second position that maintains base-pairing with the first nucleotide substitution. In an embodiment, the compensatory nucleotide substitution for a first A nucleotide substitution is a U. In an embodiment, the compensatory nucleotide substitution for a first U nucleotide substitution is an A. In an embodiment, the compensatory nucleotide substitution for a first G nucleotide substitution is a C. In an embodiment, the compensatory nucleotide substitution for a first C nucleotide substitution is a G. In an embodiment, both nucleotide positions of a base-pair comprise nucleotide substitutions, e.g., both nucleotide positions of any of base-pairs 1:72, 2:71, 3:70, 4:69, 5:68, 6:67, 7:66, 10:25, 11:24, 12:23, 13:22, 27:43, 28:42, 29:41, 30:40, 31:39, 49:65, 50:64, 51:63, 52:62, or 53:61, wherein the number preceding the colon represents the first nucleotide position of the base-pair and the number following the colon represents the second nucleotide position of the base-pair. In an embodiment, both nucleotides of the base-pair at positions 1 and 72 comprise nucleotide substitutions. In an embodiment, both nucleotides of the base-pair at positions 2 and 71 comprise nucleotide substitutions. In an embodiment, both nucleotides of the base-pair at positions 3 and 70 comprise nucleotide substitutions. In an embodiment, both nucleotides of the base-pair at positions 4 and 69 comprise nucleotide substitutions. In an embodiment, both nucleotides of the base-pair at positions 5 and 68 comprise nucleotide substitutions. In an embodiment, both nucleotides of the base-pair at positions 6 and 67 comprise nucleotide substitutions. In an embodiment, both nucleotides of the base-pair at positions 7 and 68 comprise nucleotide substitutions. In an embodiment, both nucleotides of the base-pair at positions 10 and 25 comprise nucleotide substitutions. In an embodiment, both nucleotides of the base-pair at positions 11 and 24 comprise nucleotide substitutions. In an embodiment, both nucleotides of the base-pair at positions 12 and 23 comprise nucleotide substitutions. In an embodiment, both nucleotides of the base-pair at positions 13 and 22 comprise nucleotide substitutions. In an embodiment, both nucleotides of the base-pair at positions 27 and 43 comprise nucleotide substitutions. In an embodiment, both nucleotides of the base-pair at positions 28 and 42 comprise nucleotide substitutions. In an embodiment, both nucleotides of the base-pair at positions 29 and 41 comprise nucleotide substitutions. In an embodiment, both nucleotides of the base-pair at positions 30 and 40 comprise nucleotide substitutions. In an embodiment, both nucleotides of the base-pair at positions 31 and 39 comprise nucleotide substitutions. In an embodiment, both nucleotides of the base-pair at positions 49 and 65 comprise nucleotide substitutions. In an embodiment, both nucleotides of the base-pair at positions 50 and 64 comprise nucleotide substitutions. In an embodiment, both nucleotides of the base-pair at positions 51 and 63 comprise nucleotide substitutions. In an embodiment, both nucleotides of the base-pair at positions 52 and 63 comprise nucleotide substitutions. In an embodiment, both nucleotides of the base-pair at positions 53 and 61 comprise nucleotide substitutions. In an embodiment, a TREM comprises multiple pairs of nucleotide substitutions and compensatory nucleotide substitutions, e.g., multiple pairs of nucleotide substitutions that maintain base pairing, e.g., 1, 2, 3, 4, 5, 6, 7, 8, 9, 10, or more pairs of nucleotide substitutions and compensatory nucleotide substitutions. In an embodiment, a TREM comprises 1 pair of a nucleotide substitution and a compensatory nucleotide substitution. In an embodiment, a TREM comprises 2 pairs of a nucleotide substitution and a compensatory nucleotide substitution. In an embodiment, a TREM comprises 3 pairs of a nucleotide substitution and a compensatory nucleotide substitution. In an embodiment, a TREM comprises 4 pairs of a nucleotide substitution and a compensatory nucleotide substitution. In an embodiment, a TREM comprises 5 pairs of a nucleotide substitution and a compensatory nucleotide substitution. In an embodiment, a TREM comprises 6 pairs of a nucleotide substitution and a compensatory nucleotide substitution. In an embodiment, a TREM comprises 7 pairs of a nucleotide substitution and a compensatory nucleotide substitution. In an embodiment, a TREM comprises 8 pairs of a nucleotide substitution and a compensatory nucleotide substitution. In an embodiment, a TREM comprises 9 pairs of a nucleotide substitution and a compensatory nucleotide substitution. In an embodiment, a TREM comprises 10 pairs of a nucleotide substitution and a compensatory nucleotide substitution. In an embodiment, a TREM comprises more than 10 pairs of a nucleotide substitution and a compensatory nucleotide substitution. In an embodiment, the nucleotide substitutions at a first position and a second compensatory position result in a lower energy base pair, e.g., a change from a G:C or C:G base pair to an A:U or U:A base pair. In an embodiment, the nucleotide substitutions at a first position and a second compensatory position result in a higher energy base pair, e.g., a change from an A:U or U:A base pair to a G:C or C:G base pair. In an embodiment, the nucleotide substitutions at a first position and a second compensatory position result in a neutral energy base pair, e.g., a change from an A:U base pair to a U:A base pair or a C:G base pair to a G:C base pair. In an embodiment, a nucleotide substitution results in a wobble base pair, e.g., a G:U base pair. In an embodiment, the nucleotide substitutions at a first position and a second compensatory position result in a wobble base pair, e.g., a G:U base pair. In an embodiment, a nucleotide substitution disrupts base-pairing.

In an embodiment, a nucleotide substitution results in modulation of the activity of the TREM, e.g., results in an increase or a decrease in activity of the TREM. For example, a nucleotide substitution may modulate the ability of the TREM to (i) support protein synthesis, (ii) be charged by a tRNA synthetase, (iii) be bound by an elongation factor, (iv) introduce an amino acid into a peptide chain, (v) support protein elongation, (vi) support initiation of protein synthesis, or (vii) read-through a premature termination codon (PTC), e.g., in a cell. In an embodiment, a nucleotide substitution modulates the ability of a TREM to support protein synthesis. In an embodiment, a nucleotide substitution modulates the ability of a TREM to be charged by a tRNA synthetase. In an embodiment, a nucleotide substitution modulates the ability of a TREM to be bound by an elongation factor. In an embodiment, a nucleotide substitution modulates the ability of a TREM to introduce an amino acid into a peptide chain. In an embodiment, a nucleotide substitution modulates the ability of a TREM to support protein elongation. In an embodiment, a nucleotide substitution modulates the ability of a TREM to support initiation of protein synthesis. In an embodiment, a nucleotide substitution modulates the ability of a TREM to read-through a PTC. In an embodiment, a nucleotide substitution at a particular position in the TREM results in an increase in the capability of the TREM to support protein synthesis, e.g., in a cell, e.g., by about 1%, 5%, 10%, 15%, 20%, 25%, 30%, 35%, 40%, 45%, 50%, 55%, 60%, 65%, 70%, 75%, 80%, 85%⁰, 90%, 95%, 99%, 99.5%, 99.9%, or more, e.g., relative to a reference standard. In an embodiment, a nucleotide substitution at a particular position in the TREM results in an increase in the capability of the TREM to be charged by a tRNA synthetase, e.g., in a cell, e.g., by about 1%, 5%, 10%, 15%, 20%, 25%, 30%, 35%, 40%, 45%, 50%, 55%, 60%, 65%, 70%, 75%, 80%, 85%, 90%, 95%, 99%, 99.5%, 99.9%, or more, e.g., relative to a reference standard. In an embodiment, a nucleotide substitution at a particular position in the TREM results in an increase in the capability of the TREM to be bound by an elongation factor, e.g., in a cell, e.g., by about 1%, 5%, 10%, 15%, 20%, 25%, 30%, 35%, 40%, 45%, 0%, %55%, 60%, 65%, 70%, 75%, 80%, 85%, 90%, 95%, 99%, 99.5%, 99.9%, or more, e.g., relative to a reference standard. In an embodiment, a nucleotide substitution at a particular position in the TREM results in an increase in the capability of the TREM to introduce an amino acid into a peptide chain, e.g., in a cell, e.g., by about 1%, 5%, 10%, 15%, 20%, 25%, 30%, 35%, 40%, 45%, 50%, 55%, 60%, 65%, 70%, 75%, 80%, 85%, 90%, 95%, 99%, 99.5%, 99.9%, or more, e.g., relative to a reference standard. In an embodiment, a nucleotide substitution at a particular position in the TREM results in an increase in the capability of the TREM to support protein elongation, e.g., in a cell, e.g., by about 1%, 5%, 10%, 15%, 20%, 25%, 30%, 35%, 40%, 45%, 50%, 55%, 60%, 65%, 70%, 75%, 80%, 85%, 90%, 95%, 99%, 99.5%, 99.9%, or more, e.g., relative to a reference standard. In an embodiment, a nucleotide substitution at a particular position in the TREM results in an increase in the capability of the TREM to support initiation of protein synthesis, e.g., in a cell, e.g., by about 1%, 5%, 10%, 15%, 20%, 25%, 30%, 35%, 40%, 45%, 50%, 55%, 60%, 65%, 70%, 75%, 80%, 85%, 90%, 95%, 99%, 99.5%, 99.9%, or more, e.g., relative to a reference standard. In an embodiment, a nucleotide substitution at a particular position in the TREM results in an increase in the capability of the TREM to read-through a premature termination codon, e.g., in a cell, e.g., by about 1%, 5%, 10%, 15%, 20%, 25%, 30%, 35%, 40%, 45%, 50%, 55%, 60%, 65%, 70%, 75%, 80%, 85%, 90%, 95%, 99%, 99.5%, 99.9% or more, e.g., relative to a reference standard. In an embodiment, a nucleotide substitution at a particular position in the TREM results in a decrease in the capability of the TREM to support protein synthesis, e.g., in a cell, e.g., by about 1%, 5%, 10%, 15%, 20%, 25%, 30%, 35%, 40%, 45%, 50%, 55%, 60%, 65%, 70%, 75%, 80%, 85%, 90%, 95%, 99%, 99.5%, 99.9% or more, e.g., relative to a reference standard. In an embodiment, a nucleotide substitution at a particular position in the TREM results in a decrease in the capability of the TREM to be charged by a tRNA synthetase, e.g., in a cell, e.g., by about 1%, 5%, 10%, 15%, 20%, 25%, 30%, 35%, 40%, 45%, 50%, 55%, 60%, 65%, 70%, 75%, 80%, 85%, 90%, 95%, 99%, 99.5%, 99.9% or more, e.g., relative to a reference standard. In an embodiment, a nucleotide substitution at a particular position in the TREM results in a decrease in the capability of the TREM to be bound by an elongation factor, e.g., in a cell, e.g., by about 1%, 5%, 10%, 15%, 20%, 25%, 30%, 35%, 40%, 45%, 50%, 55%, 60%, 65%, 70%, 75%, 80%, 85%, 90%, 95%, 99%, 99.5%, 99.9%, or more, e.g., relative to a reference standard. In an embodiment, a nucleotide substitution at a particular position in the TREM results in a decrease in the capability of the TREM to introduce an amino acid into a peptide chain, e.g., in a cell, e.g., by about 1%, 5%, 10%, 15%, 20%, 25%, 30%, 35%, 40%, 45%, 50%, 55%, 60%, 65%, 70%, 75%, 80%, 85%, 90%, 95%, 99%, 99.5%, 99.9%, or more, e.g., relative to a reference standard. In an embodiment, a nucleotide substitution at a particular position in the TREM results in a decrease in the capability of the TREM to support protein elongation, e.g., in a cell, e.g., by about 1%, 5%, 10%, 15%, 20%, 25%, 30%, 35%, 40%, 45%, 50%, 55%, 60%, 65%, 70%, 75%, 80%, 85%, 90%, 95%, 99%, 99.5%, 99.9%, or more, e.g., relative to a reference standard. In an embodiment, a nucleotide substitution at a particular position in the TREM results in a decrease in the capability of the TREM to support initiation of protein synthesis, e.g., in a cell, e.g., by about 1%, 5%, 10%, 15%, 20%, 25%, 30%, 35%, 40%, 45%, 50%, 55%, 60%, 65%, 70%, 75%, 80%, 85%, 90%, 95%, 99%, 99.5%, 99.9%, or more, e.g., relative to a reference standard. In an embodiment, a nucleotide substitution at a particular position in the TREM results in a decrease in the capability of the TREM to read-through a premature termination codon, e.g., in a cell, e.g., by about 1%, 5%, 10%, 15%, 20%, 25%, 30%, 35%, 40%, 45%, 50%, 55%, 60%, 65%, 70%, 75%, 80%, 85%, 90%, 95%, 99%, 99.5%, 99.9%, or more, e.g., relative to a reference standard.

In an embodiment, a nucleotide substitution results in modulation of the stability of the TREM, e.g., results in an increase or a decrease in stability of the TREM. For example, a nucleotide substitution may modulate the (i) localization of the TREM, (ii) the amount of time before the TREM is degraded, (iii) the amount of time the TREM is charged with an amino acid, (iv) the amount of time the TREM interacts with a ribosome, (v) the amount of time the TREM interacts with an elongation factor, (vi) modification of the TREM, or (vii) interaction of the TREM with tRNA degradation proteins, e.g., within a cell. In an embodiment, a nucleotide substitution modulates the localization of a TREM. In an embodiment, a nucleotide substitution modulates the amount of time before the TREM is degraded. In an embodiment, a nucleotide substitution modulates the amount of time the TREM is charged with an amino acid. In an embodiment, a nucleotide substitution modulates the amount of time the TREM interacts with a ribosome. In an embodiment, a nucleotide substitution modulates the amount of time the TREM interacts with an elongation factor. In an embodiment, a nucleotide substitution modulates modification of a TREM. In an embodiment, a nucleotide substitution modulates interaction of the TREM with tRNA degradation proteins. In an embodiment, a nucleotide substitution at a particular position in the TREM results in an increase in proper localization of a TREM, e.g., within a cell, e.g., by about 1%, 5%, 10%, 15%, 20%, 25%, 30%, 35%, 40%, 45%, 50%, 55%, 60%, 65%, 70%, 75%, 80%, 85%, 90%, 95%, 99%, 99.5%, 99.9%, or more, e.g., relative to a reference standard. In an embodiment, a nucleotide substitution at a particular position in the TREM results in an increase in the amount of time before a TREM is degraded, e.g., within a cell, e.g., by about 1%, 5%, 10%, 15%, 20%, 25%, 30%, 35%, 40%, 45%, 50%, 55%, 60%, 65%, 70%, 75%, 80%, 85%, 90%, 95%, 99%, 99.5%, 99.9%, or more, e.g., relative to a reference standard. In an embodiment, a nucleotide substitution at a particular position in the TREM results in an increase in the amount of time a TREM is charged with an amino acid, e.g., within a cell, e.g., by about 1%, 5%, 10%, 15%, 20%, 25%, 30%, 35%, 40%, 45%, 50%, 55%, 60%, 65%, 70%, 75%, 80%, 85%, 90%, 95%, 99%, 99.5%, 99.9%, or more, e.g., relative to a reference standard. In an embodiment, a nucleotide substitution at a particular position in the TREM results in an increase in the amount of time a TREM interacts with a ribosome, e.g., within a cell, e.g., by about 1%, 5%, 10%, 15%, 20%, 25%, 30%, 35%, 40%, 45%, 50%, 55%, 60%, 65%, 70%, 75%, 80%, 85%, 90%, 95%, 99%, 99.5%, 99.9%, or more, e.g., relative to a reference standard. In an embodiment, a nucleotide substitution at a particular position in the TREM results in an increase in the amount of time a TREM interacts with an elongation factor, e.g., within a cell, e.g., by about 1%, 5%, 10%, 15%, 20%, 25%, 30%, 35%, 40%, 45%, 50%, 55%, 60%, 65%, 70%, 75%, 80%, 85%, 90%, 95%, 99%, 99.5%, 99.9%, or more, e.g., relative to a reference standard. In an embodiment, a nucleotide substitution at a particular position in the TREM results in an increase in modification of a TREM, e.g., within a cell, e.g., by about 1%, 5%, 10%, 15%, 20%, 25%, 30%, 35%, 40%, 45%, 50%, 55%, 60%, 65%, 70%, 75%, 80%, 85%, 90%, 95%, 99%, 99.5%, 99.9%, or more, e.g., relative to a reference standard. In an embodiment, a nucleotide substitution at a particular position in the TREM results in an increase in interaction of the TREM with tRNA degradation proteins, e.g., within a cell, e.g., by about 1%, 5%, 10%, 15%, 20%, 25%, 30%, 35%, 40%, 45%, 50%, 55%, 60%, 65%, 70%, 75%, 80%, 85%, 90%, 95%, 99%, 99.5%, 99.9%, or more, e.g., relative to a reference standard. In an embodiment, a nucleotide substitution at a particular position in the TREM results in a decrease in proper localization of a TREM, e.g., within a cell, e.g., by about 1%, 5%, 10%, 15%, 20%, 25%, 30%, 35%, 40%, 45%, 50%, 55%, 60%, 65%, 70%, 75%, 80%, 85%, 90%, 95%, 99%, 99.5%, 99.9%, or more, e.g., relative to a reference standard. In an embodiment, a nucleotide substitution at a particular position in the TREM results in a decrease in the amount of time before a TREM is degraded, e.g., within a cell, e.g., by about 1%, 5%, 10%, 15%, 20%, 25%, 30%, 35%, 40%, 45%, 50%, 55%, 60%, 65%, 70%, 75%, 80%, 85%, 90%, 95%, 99%, 99.5%, 99.9%, or more, e.g., relative to a reference standard. In an embodiment, a nucleotide substitution at a particular position in the TREM results in a decrease in the amount of time a TREM is charged with an amino acid, e.g., within a cell, e.g., by about 1%, 5%, 10%, 15%, 20%, 25%, 30%, 35%, 40%, 45%, 50%, 55%, 60%, 65%, 70%, 75%, 80%, 85%, 90%, 95%, 99%, 99.5%, 99.9%, or more, e.g., relative to a reference standard. In an embodiment, a nucleotide substitution at a particular position in the TREM results in a decrease in the amount of time a TREM interacts with a ribosome, e.g., within a cell, e.g., by about 1%, 5%, 10%, 15%, 20%, 25%, 30%, 35%, 40%, 45%, 50%, 55%, 60%, 65%, 70%, 75%, 80%, 85%, 90%, 95%, 99%, 99.5%, 99.9%, or more, e.g., relative to a reference standard. In an embodiment, a nucleotide substitution at a particular position in the TREM results in a decrease in the amount of time a TREM interacts with an elongation factor, e.g., within a cell, e.g., by about 1%, 5%, 10%, 15%, 20%, 25%, 30%, 35%, 40%, 45%, 50%, 55%, 60%, 65%, 70%, 75%, 80%, 85%, 90%, 95%, 99%, 99.5%, 99.9%, or more, e.g., relative to a reference standard. In an embodiment, a nucleotide substitution at a particular position in the TREM results in a decrease in modification of a TREM, e.g., within a cell, e.g., by about 1%, 5%, 10%, 15%, 20%, 25%, 30%, 35%, 40%, 45%, 50%, 55%, 60%, 65%, 70%, 75%, 80%, 85%, 90%, 95%, 99%, 99.5%, 99.9%, or more, e.g., relative to a reference standard. In an embodiment, a nucleotide substitution at a particular position in the TREM results in a decrease in interaction of the TREM with tRNA degradation proteins, e.g., within a cell, e.g., by about 1%, 5%, 10%, 5%, 20%, 25%, 30%, 35%, 40%, 45%, 50%, 55%, 60%, 65%, 70%, 75%, 80%, 85%, 90%, 95%, 99%, 99.5%, 99.9%, or more, e.g., relative to a reference standard.

Exemplary nucleotide sequence modifications are outlined below.

A TREM may comprise a nucleotide mutation relative to the nucleotide sequence of SEQ ID NO: 734. In an embodiment, the nucleotide mutation relative to the nucleotide sequence of SEQ ID NO: 734 is a nucleotide substitution. In an embodiment, the TREM comprises a nucleotide substitution relative to the nucleotide sequence of SEQ ID NO: 734 in the [VL Domain]. In an embodiment, the TREM comprises one nucleotide substitution. In an embodiment, the nucleotide substitution is at a nucleotide position between positions 40 and 50. In an embodiment, the nucleotide substitution is at nucleotide position 40, 41, 42, 43, 44, 45, 46, 47, 48, 49, or 50. In an embodiment, the nucleotide substitution is at position 44. In an embodiment, the nucleotide substitution is an A to G substitution. In an embodiment, the nucleotide substitution is A44G. In an embodiment, the TREM has the nucleotide sequence of SEQ ID NO: 743. In an embodiment, the TREM having SEQ ID NO: 743 comprises a non-naturally occurring modification. In an embodiment, the TREM having SEQ ID NO: 743 has a non-naturally occurring modification listed in Table 6, e.g., one of Pattern Nos: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, and 32.

A TREM may comprise a nucleotide substitution relative to the nucleotide sequence of SEQ ID NO: 734, wherein the nucleotide substitution relative to the nucleotide sequence of SEQ ID NO: 734 is present in each of [ASt Domain1] and the [ASt Domain2]. In an embodiment, the TREM comprises eight nucleotide substitutions. In an embodiment, the nucleotide substitution is at a nucleotide position between positions 1 and 10. In an embodiment, the nucleotide substitution is at a nucleotide position between positions 60 and 70. In an embodiment, the nucleotide substitution is at a nucleotide position between positions 70 and 80. In an embodiment, the nucleotide substitution is at nucleotide position 1, 2, 3, 4, 5, 6, 7, 8, 9, or 10. In an embodiment, the nucleotide substitution is at nucleotide position 60, 61, 62, 63, 64, 65, 66, 67, 68, 69, or 70. In an embodiment, the nucleotide substitution is at nucleotide position 70, 71, 72, 73, 74, 75, 76, 77, 78, 79, or 80. In an embodiment, the nucleotide substitution is at position 2. In an embodiment, the nucleotide substitution is at position 4. In an embodiment, the nucleotide substitution is at position 5. In an embodiment, the nucleotide substitution is at position 6. In an embodiment, the nucleotide substitution is at position 67. In an embodiment, the nucleotide substitution is at position 68. In an embodiment, the nucleotide substitution is at position 69. In an embodiment, the nucleotide substitution is at position 71. In an embodiment, the nucleotide substitution is a G to C substitution. In an embodiment, the nucleotide substitution is a U to C substitution. In an embodiment, the nucleotide substitution is a C to G substitution. In an embodiment, the nucleotide substitution is a C to A substitution. In an embodiment, the nucleotide substitution is a G to U substitution. In an embodiment, the nucleotide substitution is an A to G substitution. In an embodiment, the nucleotide substitution is a U to G substitution. In an embodiment, the nucleotide substitution is G2C. In an embodiment, the nucleotide substitution is U4C. In an embodiment, the nucleotide substitution is C5G. In an embodiment, the nucleotide substitution is C6A. In an embodiment, the nucleotide substitution is G67U. In an embodiment, the nucleotide substitution is G68C. In an embodiment, the nucleotide substitution is A69G. In an embodiment, the nucleotide substitution is U71G. In an embodiment, the TREM has the nucleotide sequence of SEQ ID NO: 639. In an embodiment, the TREM having SEQ ID NO: 639 comprises a non-naturally occurring modification. In an embodiment, the TREM having SEQ ID NO: 639 has a non-naturally occurring modification listed in Table 6, e.g., one of Pattern Nos: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, and 32.

A TREM may comprise a nucleotide substitution relative to the nucleotide sequence of SEQ ID NO: 734, wherein the nucleotide substitution is present in each of the [ASt Domain1], the [DH Domain], the [ACH Domain], the [VL Domain], and the [ASt Domain2]. In an embodiment, the TREM comprises seventeen nucleotide substitutions. In an embodiment, the nucleotide substitution is at a nucleotide position between positions 1 and 10. In an embodiment, the nucleotide substitution is at a nucleotide position between positions 10 and 20. In an embodiment, the nucleotide substitution is at a nucleotide position between positions 20 and 30. In an embodiment, the nucleotide substitution is at a nucleotide position between positions 30 and 40. In an embodiment, the nucleotide substitution is at a nucleotide position between positions 40 and 50. In an embodiment, the nucleotide substitution is at a nucleotide position between positions 60 and 70. In an embodiment, the nucleotide substitution is at a nucleotide position between positions 70 and 80. In an embodiment, the nucleotide substitution is at nucleotide position 1, 2, 3, 4, 5, 6, 7, 8, 9, or 10. In an embodiment, the nucleotide substitution is at nucleotide position 10, 11, 12, 13, 14, 15, 16, 17, 18, 19, or 20. In an embodiment, the nucleotide substitution is at nucleotide position 20, 21, 22, 23, 24, 25, 26, 27, 28, 29, or 30. In an embodiment, the nucleotide substitution is at nucleotide position 30, 31, 32, 33, 34, 35, 36, 37, 38, 39, or 40. In an embodiment, the nucleotide substitution is at nucleotide position 40, 41, 41, 43, 44, 45, 46, 47, 48, 49, or 50. In an embodiment, the nucleotide substitution is at nucleotide position 60, 61, 62, 63, 64, 65, 66, 67, 68, 69, and 70. In an embodiment, the nucleotide substitution is at nucleotide position 70, 71, 72, 73, 74, 75, 76, 77, 78, 79, or 80. In an embodiment, the nucleotide substitution is at position 2. In an embodiment, the nucleotide substitution is at position 12. In an embodiment, the nucleotide substitution is at position 13. In an embodiment, the nucleotide substitution is at position 22. In an embodiment, the nucleotide substitution is at position 23. In an embodiment, the nucleotide substitution is at position 28. In an embodiment, the nucleotide substitution is at position 31. In an embodiment, the nucleotide substitution is at position 39. In an embodiment, the nucleotide substitution is at position 40. In an embodiment, the nucleotide substitution is at position 42. In an embodiment, the nucleotide substitution is at position 43. In an embodiment, the nucleotide substitution is at position 44. In an embodiment, the nucleotide substitution is at position 46. In an embodiment, the nucleotide substitution is at position 49. In an embodiment, the nucleotide substitution is at position 65. In an embodiment, the nucleotide substitution is at position 71. In an embodiment, the nucleotide substitution is at position 72. In an embodiment, the nucleotide substitution is a G to C substitution. In an embodiment, the nucleotide substitution is a C to U substitution. In an embodiment, the nucleotide substitution is a G to A substitution. In an embodiment, the nucleotide substitution is a C to G substitution. In an embodiment, the nucleotide substitution is a U to C substitution. In an embodiment, the nucleotide substitution is an A to C substitution. In an embodiment, the nucleotide substitution is a U to G substitution. In an embodiment, the nucleotide substitution is an A to G substitution. In an embodiment, the nucleotide substitution is a C to G substitution. In an embodiment, the nucleotide substitution is a G to U substitution. In an embodiment, the nucleotide substitution is G2C. In an embodiment, the nucleotide substitution is G12C. In an embodiment, the nucleotide substitution is C13U. In an embodiment, the nucleotide substitution is G22A. In an embodiment, the nucleotide substitution is C23G. In an embodiment, the nucleotide substitution is U28C. In an embodiment, the nucleotide substitution is A3TC. In an embodiment, the nucleotide substitution is U39G. In an embodiment, the nucleotide substitution is U40C. In an embodiment, the nucleotide substitution is A42G. In an embodiment, the nucleotide substitution is A43G. In an embodiment, the nucleotide substitution is A44G. In an embodiment, the nucleotide substitution is G46A. In an embodiment, the nucleotide substitution is C49G. In an embodiment, the nucleotide substitution is G65U. In an embodiment, the nucleotide substitution is U71G. In an embodiment, the nucleotide substitution is C72U. In an embodiment, the TREM has the nucleotide sequence of SEQ ID NO: 1149. In an embodiment, the TREM having SEQ ID NO: 1149 comprises a non-naturally occurring modification. In an embodiment, the TREM having SEQ ID NO: 1149 has a non-naturally occurring modification listed in Table 6, e.g., one of Pattern Nos: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, and 32.

A TREM may comprise a nucleotide substitution relative to the nucleotide sequence of SEQ ID NO: 734, wherein the nucleotide substitution is present in each of the [ASt Domain1] and the [ASt Domain2]. In an embodiment, the TREM comprises four nucleotide substitutions. In an embodiment, the nucleotide substitution is at a nucleotide position between positions 1 and 10. In an embodiment, the nucleotide substitution is at a nucleotide position between positions 60 and 70. In an embodiment, the nucleotide substitution is at nucleotide position 1, 2, 3, 4, 5, 6, 7, 8, 9, or 10. In an embodiment, the nucleotide substitution is at nucleotide position 60, 61, 62, 63, 64, 64, 65, 66, 67, 68, 69, or 70. In an embodiment, the nucleotide substitution is at position 4. In an embodiment, the nucleotide substitution is at position 7. In an embodiment, the nucleotide substitution is at position 66. In an embodiment, the nucleotide substitution is at position 69. In an embodiment, the nucleotide substitution is a U to C substitution. In an embodiment, the nucleotide substitution is a G to A substitution. In an embodiment, the nucleotide substitution is a C to U substitution. In an embodiment, the nucleotide substitution is an A to G substitution. In an embodiment, the nucleotide substitution is U4C. In an embodiment, the nucleotide substitution is G7A. In an embodiment, the nucleotide substitution is C66U. In an embodiment, the nucleotide substitution is A69G. In an embodiment, the TREM has the nucleotide sequence of SEQ ID NO: 652. In an embodiment, the TREM having SEQ ID NO: 652 comprises a non-naturally occurring modification. In an embodiment, the TREM having SEQ ID NO: 652 has a non-naturally occurring modification listed in Table 6, e.g., one of Pattern Nos: 1, 2, 3, 4, 5, 6, 7, 8,9, 10, 11, 12, 13, 14, 15, 16, 17, 18, 19, 20, 21, 22, 23, 24, 25, 26, 27, 28, 29, 30, 31, and 32. A TREM may comprise a nucleotide substitution relative to the nucleotide sequence of SEQ ID NO: 734, wherein the nucleotide substitution is present in the [ACH Domain]. In an embodiment, the TREM comprises nine nucleotide substitutions. In an embodiment, the nucleotide substitution is at a nucleotide position between positions 20 and 30. In an embodiment, the nucleotide substitution is at a nucleotide position between positions 30 and 40. In an embodiment, the nucleotide substitution is at a nucleotide position between positions 40 and 50. In an embodiment, the nucleotide substitution is at nucleotide position 20, 21, 22, 23, 24, 25, 26, 27, 28, 29, or 30. In an embodiment, the nucleotide substitution is at nucleotide position 30, 31, 32, 33, 34, 35, 36, 37, 38, 39, or 40. In an embodiment, the nucleotide substitution is at nucleotide position 40, 41, 42, 43, 44, 45, 46, 47, 48, 49, or 50. In an embodiment, the nucleotide substitution is at position 27. In an embodiment, the nucleotide substitution is at position 28. In an embodiment, the nucleotide substitution is at position 29. In an embodiment, the nucleotide substitution is at position 31. In an embodiment, the nucleotide substitution is at position 39. In an embodiment, the nucleotide substitution is at position 40. In an embodiment, the nucleotide substitution is at position 41. In an embodiment, the nucleotide substitution is at position 42. In an embodiment, the nucleotide substitution is at position 43. In an embodiment, the nucleotide substitution is a U to A substitution. In an embodiment, the nucleotide substitution is a U to C substitution. In an embodiment, the nucleotide substitution is a G to C substitution. In an embodiment, the nucleotide substitution is an A to U substitution. In an embodiment, the nucleotide substitution is a C to G substitution. In an embodiment, the nucleotide substitution is an A to G substitution. In an embodiment, the nucleotide substitution is U27A. In an embodiment, the nucleotide substitution is U28C. In an embodiment, the nucleotide substitution is G29C. In an embodiment, the nucleotide substitution is A31U. In an embodiment, the nucleotide substitution is U39A. In an embodiment, the nucleotide substitution is U40C. In an embodiment, the nucleotide substitution is C41G. In an embodiment, the nucleotide substitution is A42G. In an embodiment, the nucleotide substitution is A43U. In an embodiment, the TREM has the nucleotide sequence of SEQ ID NO: 702. In an embodiment, the TREM having SEQ ID NO: 702 comprises a non-naturally occurring modification. In an embodiment, the TREM having SEQ ID NO: 702 has a non-naturally occurring modification listed in Table 6, e.g., one of Pattern Nos: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, and 32.

A TREM may comprise a nucleotide substitution relative to the nucleotide sequence of SEQ ID NO: 734, wherein the nucleotide substitution is present in each of the [ASt Domain1] and the [ASt Domain2]. In an embodiment, the TREM comprises eight nucleotide substitutions. In an embodiment, the nucleotide substitution is at a nucleotide position between positions 1 and 10. In an embodiment, the nucleotide substitution is at a nucleotide position between positions 60 and 70. In an embodiment, the nucleotide substitution is at a nucleotide position between positions 70 and 80. In an embodiment, the nucleotide substitution is at nucleotide position 1, 2, 3, 4, 5, 6, 7, 8, 9, or 10. In an embodiment, the nucleotide substitution is at nucleotide position 60, 61, 62, 63, 64, 65, 66, 67, 68, 69, or 70. In an embodiment, the nucleotide substitution is at nucleotide position 70, 71, 72, 73, 74, 75, 76, 77, 78, 79, or 80. In an embodiment, the nucleotide substitution is at position 1. In an embodiment, the nucleotide substitution is at position 2. In an embodiment, the nucleotide substitution is at position 4. In an embodiment, the nucleotide substitution is at position 5. In an embodiment, the nucleotide substitution is at position 68. In an embodiment, the nucleotide substitution is at position 69. In an embodiment, the nucleotide substitution is at position 71. In an embodiment, the nucleotide substitution is at position 72. In an embodiment, the nucleotide substitution is a G to A substitution. In an embodiment, the nucleotide substitution is a G to C substitution. In an embodiment, the nucleotide substitution is a U to A substitution. In an embodiment, the nucleotide substitution is a C to A substitution. In an embodiment, the nucleotide substitution is a G to U substitution. In an embodiment, the nucleotide substitution is an A to U substitution. In an embodiment, the nucleotide substitution is a U to G substitution. In an embodiment, the nucleotide substitution is a C to U substitution. In an embodiment, the nucleotide substitution is G1A. In an embodiment, the nucleotide substitution is G2C. In an embodiment, the nucleotide substitution is U4A. In an embodiment, the nucleotide substitution is C5A. In an embodiment, the nucleotide substitution is G68U. In an embodiment, the nucleotide substitution is A69U. In an embodiment, the nucleotide substitution is U71G. In an embodiment, the nucleotide substitution is C72U. In an embodiment, the TREM has the nucleotide sequence of SEQ ID NO: 627. In an embodiment, the TREM having SEQ ID NO: 627 comprises a non-naturally occurring modification. In an embodiment, the TREM having SEQ ID NO: 627 has a non-naturally occurring modification listed in Table 6, e.g., one of Pattern Nos: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, and 32.

A TREM may comprise a nucleotide substitution relative to the nucleotide sequence of SEQ ID NO: 734, wherein the nucleotide substitution is present in each of the [ASt Domain1], the [DH Domain], the [VL Domain], and the [ASt Domain2]. In an embodiment, the TREM comprises five nucleotide substitutions. In an embodiment, the nucleotide substitution is at a nucleotide position between positions 1 and 10. In an embodiment, the nucleotide substitution is at a nucleotide position between positions 20 and 30. In an embodiment, the nucleotide substitution is at a nucleotide position between positions 40 and 50. In an embodiment, the nucleotide substitution is at a nucleotide position between positions 60 and 70. In an embodiment, the nucleotide substitution is at nucleotide position 1, 2, 3, 4, 5, 6, 7, 8, 9, or 10. In an embodiment, the nucleotide substitution is at nucleotide position 20, 21, 22, 23, 24, 25, 26, 27, 28, 29, or 30. In an embodiment, the nucleotide substitution is at nucleotide position 40, 41, 42, 43, 44, 45, 46, 47, 48, 49, or 50. In an embodiment, the nucleotide substitution is at nucleotide position 60, 61, 62, 63, 64, 65, 66, 67, 68, 69, or 70. In an embodiment, the nucleotide substitution is at position 4. In an embodiment, the nucleotide substitution is at position 26. In an embodiment, the nucleotide substitution is at position 49. In an embodiment, the nucleotide substitution is at position 65. In an embodiment, the nucleotide substitution is at position 69. In an embodiment, the nucleotide substitution is a U to C substitution. In an embodiment, the nucleotide substitution is an A to G substitution. In an embodiment, the nucleotide substitution is a C to G substitution. In an embodiment, the nucleotide substitution is a G to C substitution. In an embodiment, the nucleotide substitution is U4C. In an embodiment, the nucleotide substitution is A26G. In an embodiment, the nucleotide substitution is C49G. In an embodiment, the nucleotide substitution is G65C. In an embodiment, the nucleotide substitution is A69G. In an embodiment, the TREM has the nucleotide sequence of SEQ ID NO: 1150. In an embodiment, the TREM having SEQ ID NO: 1150 comprises a non-naturally occurring modification. In an embodiment, the TREM having SEQ ID NO: 1150 has a non-naturally occurring modification listed in Table 6, e.g., one of Pattern Nos: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, and 32.

A TREM may comprise a nucleotide substitution relative to the nucleotide sequence of SEQ ID NO: 734, wherein the nucleotide substitution is present in each of the [VL Domain] and the [ASt Domain2]. In an embodiment, the TREM comprises two nucleotide substitutions. In an embodiment, the nucleotide substitution is at a nucleotide position between positions 40 and 50. In an embodiment, the nucleotide substitution is at a nucleotide position between positions 60 and 70. In an embodiment, the nucleotide substitution is at nucleotide position 40, 41, 42, 43, 44, 45, 46, 47, 48, 49, or 50. In an embodiment, the nucleotide substitution is at nucleotide position 60, 61, 62, 63, 64, 65, 66, 67, 68, 69, or 70. In an embodiment, the nucleotide substitution is at position 49. In an embodiment, the nucleotide substitution is at position 65. In an embodiment, the nucleotide substitution is a C to G substitution. In an embodiment, the nucleotide substitution is a G to C substitution. In an embodiment, the nucleotide substitution is C49G. In an embodiment, the nucleotide substitution is G65C. In an embodiment, the TREM has the nucleotide sequence of SEQ ID NO: 1151. In an embodiment, the TREM having SEQ ID NO: 1151 comprises a non-naturally occurring modification. In an embodiment, the TREM having SEQ ID NO: 1151 has a non-naturally occurring modification listed in Table 6, e.g., one of Pattern Nos: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, and 32.

A TREM may comprise a nucleotide substitution relative to the nucleotide sequence of SEQ ID NO: 734, wherein the nucleotide substitution is present in each of the [ASt Domain1] and the [ASt Domain2]. In an embodiment, the TREM comprises five nucleotide substitutions. In an embodiment, the nucleotide substitution is at a nucleotide position between positions 1 and 10. In an embodiment, the nucleotide substitution is at a nucleotide position between positions 60 and 70. In an embodiment, the nucleotide substitution is at a nucleotide position between positions 70 and 80. In an embodiment, the nucleotide substitution is at nucleotide position 1, 2, 3, 4, 5, 6, 7, 8, 9, or 10. In an embodiment, the nucleotide substitution is at nucleotide position 60, 61, 62, 63, 64, 65, 66, 67, 68, 69, or 70. In an embodiment, the nucleotide substitution is at nucleotide position 70, 71, 72, 73, 74, 75, 76, 77, 78, 79, or 80. In an embodiment, the nucleotide substitution is at position 1. In an embodiment, the nucleotide substitution is at position 2. In an embodiment, the nucleotide substitution is at position 4. In an embodiment, the nucleotide substitution is at position 69. In an embodiment, the nucleotide substitution is at position 72. In an embodiment, the nucleotide substitution is a G to U substitution. In an embodiment, the nucleotide substitution is a G to A substitution. In an embodiment, the nucleotide substitution is a U to A substitution. In an embodiment, the nucleotide substitution is an A to U substitution. In an embodiment, the nucleotide substitution is a C to A substitution. In an embodiment, the nucleotide substitution is G1U. In an embodiment, the nucleotide substitution is G2A. In an embodiment, the nucleotide substitution is U4A. In an embodiment, the nucleotide substitution is A69U. In an embodiment, the nucleotide substitution is C72A. In an embodiment, the TREM has the nucleotide sequence of SEQ ID NO: 794. In an embodiment, the TREM having SEQ ID NO: 794 comprises a non-naturally occurring modification. In an embodiment, the TREM having SEQ ID NO: 794 has a non-naturally occurring modification listed in Table 6, e.g., one of Pattern Nos: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, and 32.

A TREM may comprise a nucleotide substitution relative to the nucleotide sequence of SEQ ID NO: 734, wherein the nucleotide substitution is present in each of the [ASt Domain1], the [ACH Domain], and the [ASt Domain2]. In an embodiment, the TREM comprises four nucleotide substitutions. In an embodiment, the nucleotide substitution is at a nucleotide position between positions 1 and 10. In an embodiment, the nucleotide substitution is at a nucleotide position between positions 20 and 30. In an embodiment, the nucleotide substitution is at a nucleotide position between positions 40 and 50. In an embodiment, the nucleotide substitution is at a nucleotide position between positions 70 and 80. In an embodiment, the nucleotide substitution is at nucleotide position 1, 2, 3, 4, 5, 6, 7, 8, 9, or 10. In an embodiment, the nucleotide substitution is at nucleotide position 20, 21, 22, 23, 24, 25, 26, 27, 28, 29, or 30. In an embodiment, the nucleotide substitution is at nucleotide position 40, 41, 42, 43, 44, 45, 46, 47, 48, 49, or 50. In an embodiment, the nucleotide substitution is at nucleotide position 70, 71, 72, 73, 74, 75, 76, 77, 78, 79, or 80. In an embodiment, the nucleotide substitution is at position 2. In an embodiment, the nucleotide substitution is at position 27. In an embodiment, the nucleotide substitution is at position 43. In an embodiment, the nucleotide substitution is at position 71. In an embodiment, the nucleotide substitution is a G to C substitution. In an embodiment, the nucleotide substitution is a U to G substitution. In an embodiment, the nucleotide substitution is an A to C substitution. In an embodiment, the nucleotide substitution is G2C. In an embodiment, the nucleotide substitution is U27G. In an embodiment, the nucleotide substitution is A43C. In an embodiment, the nucleotide substitution is U71G. In an embodiment, the TREM has the nucleotide sequence of SEQ ID NO: 641. In an embodiment, the TREM having SEQ ID NO: 641 comprises a non-naturally occurring modification. In an embodiment, the TREM having SEQ ID NO: 641 has a non-naturally occurring modification listed in Table 6, e.g., one of Pattern Nos: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, and 32.

A TREM may comprise a nucleotide substitution relative to the nucleotide sequence of SEQ ID NO: 734, wherein the nucleotide substitution is present in the [ACH Domain]. In an embodiment, the TREM comprises four nucleotide substitutions. In an embodiment, the nucleotide substitution is at a nucleotide position between positions 20 and 30. In an embodiment, the nucleotide substitution is at a nucleotide position between positions 30 and 40. In an embodiment, the nucleotide substitution is at a nucleotide position between positions 40 and 50. In an embodiment, the nucleotide substitution is at nucleotide position 20, 21, 22, 23, 24, 25, 26, 27, 28, 29, or 30. In an embodiment, the nucleotide substitution is at nucleotide position 30, 31, 32, 33, 34, 35, 36, 37, 38, 39, or 40. In an embodiment, the nucleotide substitution is at nucleotide position 40, 41, 42, 43, 44, 45, 46, 47, 48, 49, or 50. In an embodiment, the nucleotide substitution is at position 28. In an embodiment, the nucleotide substitution is at position 31. In an embodiment, the nucleotide substitution is at position 39. In an embodiment, the nucleotide substitution is at position 42. In an embodiment, the nucleotide substitution is a U to C substitution. In an embodiment, the nucleotide substitution is an A to U substitution. In an embodiment, the nucleotide substitution is a U to A substitution. In an embodiment, the nucleotide substitution is an A to G substitution. In an embodiment, the nucleotide substitution is U28C. In an embodiment, the nucleotide substitution is A31U. In an embodiment, the nucleotide substitution is U39A. In an embodiment, the nucleotide substitution is A42G. In an embodiment, the TREM has the nucleotide sequence of SEQ ID NO: 710. In an embodiment, the TREM having SEQ ID NO: 710 comprises a non-naturally occurring modification. In an embodiment, the TREM having SEQ ID NO: 710 has a non-naturally occurring modification listed in Table 6, e.g., one of Pattern Nos: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31 and 32.

TABLE 7
Exemplary TREM sequences

| | | Substitutions | |
|---|---|---|---|
| | SEQ | Relative to | |
| Scaffold | ID | SEQ ID NO: | |
| No. | NO. | 734 | Nucleotide Mutation Pattern |
| 1 | 743 | 1 | A44G |
| 2 | 639 | 8 | G2C, U4C, C5G, C6A, G67U, G68C, |
| | | | A69G, U71G |
| 3 | 1149 | 17 | G2C, G12C, C13U, G22A, C23G, U28C, |
| | | | A31C, U39G, U40C, A42G, A43G, A44G, |
| | | | G46A, C49G, G65U, U71G, C72U |
| 4 | 652 | 4 | U4C, G7A, C66U, A69G |
| 5 | 702 | 9 | U27A, U28C, G29C, A31U, U39A, U40C, |
| | | | C41G, A42G, A43U |
| 6 | 627 | 8 | GIA, G2C, U4A, C5A, G68U, A69U, |
| | | | U71G, C72U |
| 7 | 1150 | 5 | U4C, A26G, C49G, G65C, A69G |
| 8 | 1151 | 2 | C49G, G65C |
| 9 | 794 | 5 | GIU, G2A, U4A, A69U, C72A |
| 10 | 641 | 4 | G2C, U27G, A43C, U71G |
| 11 | 710 | 4 | U28C, A31U, U39A, A42G |
| 12 | 734 | 0 | Reference sequence. |

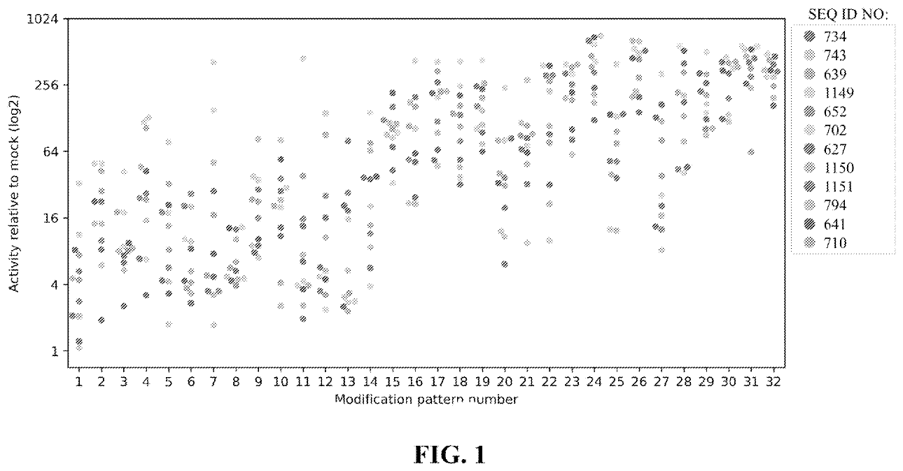

In an embodiment, a TREM may comprise a nucleotide sequence scaffold, e.g., any one of Scaffold Nos: 1, 2, 3, 4, 5, 6, 7, 8, 9, 10, 11, and 12. In an embodiment, a TREM may comprise a chemical modification pattern, e.g., any one of Pattern Nos: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, and 32. In an embodiment, a TREM may comprise a nucleotide sequence scaffold, e.g., any one of Scaffold Nos: 1, 2, 3, 4, 5, 6, 7, 8, 9, 10, 11, and 12, and a chemical modification pattern, e.g., any one of Pattern Nos: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, and 32. In an embodiment, a functional parameter of a TREM may be modulated by the sequence scaffold. In an embodiment, a functional parameter of a TREM may be modulated by the chemical modification pattern. In an embodiment, a functional parameter of a TREM may be modulated by both the sequence scaffold and the modification pattern. In an embodiment, the functional parameter of the TREM that is modulated by the nucleotide sequence scaffold, the chemical modification pattern, or both the nucleotide sequence scaffold and the chemical modification pattern is the log 2 activity of the TREM relative to mock, e.g., log 2 activity of the TREM as provided in FIG. 1. In an embodiment, the log 2 activity of a TREM can be modulated to be between 1 and 1024. In an embodiment, the log 2 activity of a TREM can be modulated to be between 1-4, 4-16, 16-64, 64-256, or 256-1024. In an embodiment, the log 2 activity of a TREM can be modulated to be between about 1 to about 40. In an embodiment, the log 2 activity of a TREM can be modulated to be between about 2 to about 60. In an embodiment, the log 2 activity of a TREM can be modulated to be between about 3 to about 60. In an embodiment, the log 2 activity of a TREM can be modulated to be between about 3 to about 50. In an embodiment, the log 2 activity of a TREM can be modulated to be between about 2 to about 128. In an embodiment, the log 2 activity of a TREM can be modulated to be between about 3 to about 30. In an embodiment, the log 2 activity of a TREM can be modulated to be between about 3 to about 512. In an embodiment, the log 2 activity of a TREM can be modulated to be between about 4 to about 15. In an embodiment, the log 2 activity of a TREM can be modulated to be between about 8 to about 100. In an embodiment, the log 2 activity of a TREM can be modulated to be between about 3 to about 70. In an embodiment, the log 2 activity of a TREM can be modulated to be between about 3 to about 512. In an embodiment, the log 2 activity of a TREM can be modulated to be between about 3 to about 128. In an embodiment, the log 2 activity of a TREM can be modulated to be between about 3 to about 70. In an embodiment, the log 2 activity of a TREM can be modulated to be between about 4 to about 128. In an embodiment, the log 2 activity of a TREM can be modulated to be between about 32 to about 256. In an embodiment, the log 2 activity of a TREM can be modulated to be between about 30 to about 512. In an embodiment, the log 2 activity of a TREM can be modulated to be between about 60 to about 512. In an embodiment, the log 2 activity of a TREM can be modulated to be between about 32 to about 512. In an embodiment, the log 2 activity of a TREM can be modulated to be between about 64 to about 512. In an embodiment, the log 2 activity of a TREM can be modulated to be between about 8 to about 256. In an embodiment, the log 2 activity of a TREM can be modulated to be between about 10 to about 256. In an embodiment, the log 2 activity of a TREM can be modulated to be between about 10 to about 400. In an embodiment, the log 2 activity of a TREM can be modulated to be between about 64 to about 500. In an embodiment, the log 2 activity of a TREM can be modulated to be between about 128 to about 800. In an embodiment, the log 2 activity of a TREM can be modulated to be between about 14 to about 400. In an embodiment, the log 2 activity of a TREM can be modulated to be between about 128 to about 700. In an embodiment, the log 2 activity of a TREM can be modulated to be between about 8 to about 300. In an embodiment, the log 2 activity of a TREM can be modulated to be between about 40 to about 512. In an embodiment, the log 2 activity of a TREM can be modulated to be between about 100 to about 512. In an embodiment, the log 2 activity of a TREM can be modulated to be between about 128 to about 400. In an embodiment, the log 2 activity of a TREM can be modulated to be between about 64 to about 512. In an embodiment, the log 2 activity of a TREM can be modulated to be between about 200 to about 512. In an embodiment, the log 2 activity of a TREM can be modulated by Pattern No: 1 to be between about 1 to about 40. In an embodiment, the log 2 activity of a TREM can be modulated by Pattern No: 2 to be between about 2 to about 60. In an embodiment, the log 2 activity of a TREM can be modulated by Pattern No: 3 to be between about 3 to about 60. In an embodiment, the log 2 activity of a TREM can be modulated by Pattern No: 4 to be between about 3 to about 50. In an embodiment, the log 2 activity of a TREM can be modulated by Pattern No: 5 to be between about 2 to about 128. In an embodiment, the log 2 activity of a TREM can be modulated by Pattern No: 6 to be between about 3 to about 30. In an embodiment, the log 2 activity of a TREM can be modulated by Pattern No: 7 to be between about 3 to about 512. In an embodiment, the log 2 activity of a TREM can be modulated by Pattern No: 8 to be between about 4 to about 15. In an embodiment, the log 2 activity of a TREM can be modulated by Pattern No: 9 to be between about 8 to about 100. In an embodiment, the log 2 activity of a TREM can be modulated by Pattern No: 10 to be between about 3 to about 70. In an embodiment, the log 2 activity of a TREM can be modulated by Pattern No: 11 to be between about 3 to about 512. In an embodiment, the log 2 activity of a TREM can be modulated by Pattern No: 12 to be between about 3 to about 128. In an embodiment, the log 2 activity of a TREM can be modulated by Pattern No: 13 to be between about 3 to about 70. In an embodiment, the log 2 activity of a TREM can be modulated by Pattern No: 14 to be between about 4 to about 128. In an embodiment, the log 2 activity of a TREM can be modulated by Pattern No: 15 to be between about 32 to about 256. In an embodiment, the log 2 activity of a TREM can be modulated by Pattern No: 16 to be between about 30 to about 512. In an embodiment, the log 2 activity of a TREM can be modulated by Pattern No: 17 to be between about 60 to about 512. In an embodiment, the log 2 activity of a TREM can be modulated by Pattern No: 18 to be between about 32 to about 512. In an embodiment, the log 2 activity of a TREM can be modulated by Pattern No: 19 to be between about 64 to about 512. In an embodiment, the log 2 activity of a TREM can be modulated by Pattern No: 20 to be between about 8 to about 256. In an embodiment, the log 2 activity of a TREM can be modulated by Pattern No: 21 to be between about 10 to about 256. In an embodiment, the log 2 activity of a TREM can be modulated by Pattern No: 22 to be between about 10 to about 400. In an embodiment, the log 2 activity of a TREM can be modulated by Pattern No: 23 to be between about 64 to about 500. In an embodiment, the log 2 activity of a TREM can be modulated by Pattern No: 24 to be between about 128 to about 800. In an embodiment, the log 2 activity of a TREM can be modulated by Pattern No: 25 to be between about 14 to about 400. In an embodiment, the log 2 activity of a TREM can be modulated by Pattern No: 26 to be between about 128 to about 700. In an embodiment, the log 2 activity of a TREM can be modulated by Pattern No: 27 to be between about 8 to about 300. In an embodiment, the log 2 activity of a TREM can be modulated by Pattern No: 28 to be between about 40 to about 512. In an embodiment, the log 2 activity of a TREM can be modulated by Pattern No: 29 to be between about 100 to about 512. In an embodiment, the log 2 activity of a TREM can be modulated by Pattern No: 30 to be between about 128 to about 400. In an embodiment, the log 2 activity of a TREM can be modulated by Pattern No: 31 to be between about 64 to about 512. In an embodiment, the log 2 activity of a TREM can be modulated by Pattern No: 32 to be between about 200 to about 512. In an embodiment, the log 2 activity of a TREM can be modulated by Scaffold No: 1 to be between about 1 to about 800. In an embodiment, the log 2 activity of a TREM can be modulated by Scaffold No: 2 to be between about 4 to about 400. In an embodiment, the log 2 activity of a TREM can be modulated by Scaffold No: 3 to be between about 3 to about 512. In an embodiment, the log 2 activity of a TREM can be modulated by Scaffold No: 4 to be between about 3 to about 400. In an embodiment, the log 2 activity of a TREM can be modulated by Scaffold No: 5 to be between about 3 to about 512. In an embodiment, the log 2 activity of a TREM can be modulated by Scaffold No: 6 to be between about 2 to about 256. In an embodiment, the log 2 activity of a TREM can be modulated by Scaffold No: 7 to be between about 3 to about 700. In an embodiment, the log 2 activity of a TREM can be modulated by Scaffold No: 8 to be between about 2 to about 600. In an embodiment, the log 2 activity of a TREM can be modulated by Scaffold No: 9 to be between about 3 to about 200. In an embodiment, the log 2 activity of a TREM can be modulated by Scaffold No: 10 to be between about 3 to about 800. In an embodiment, the log 2 activity of a TREM can be modulated by Scaffold No: 11 to be between about 3 to about 300. In an embodiment, the log 2 activity of a TREM can be modulated by Scaffold No: 12 to be between about 1 to about 400.

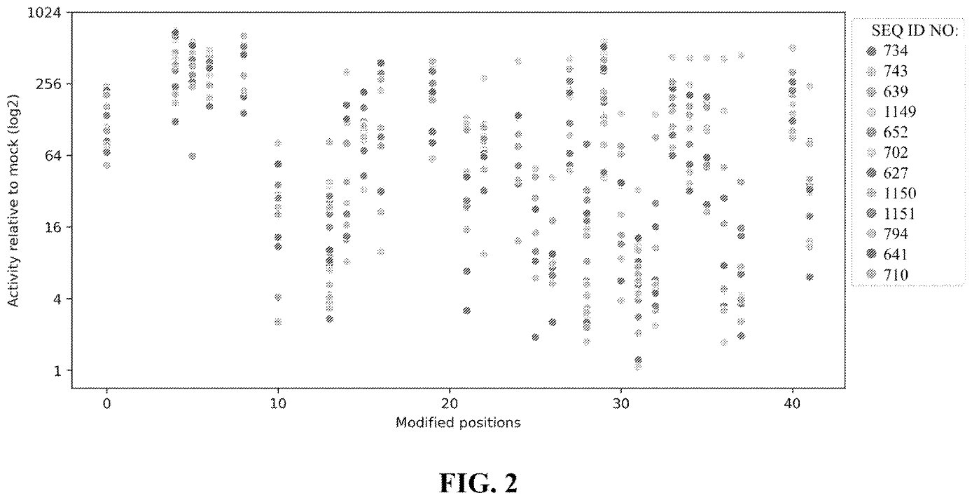

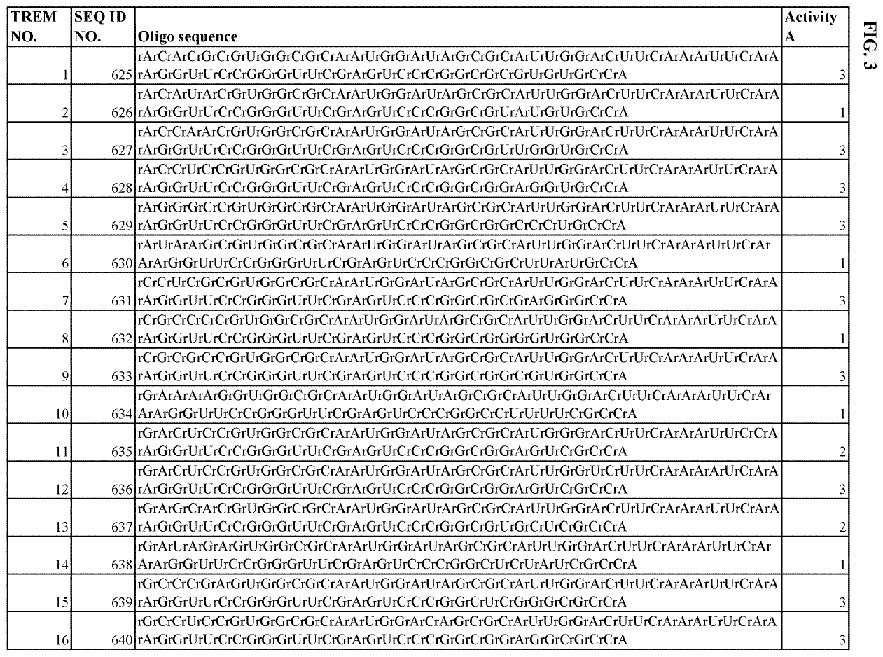

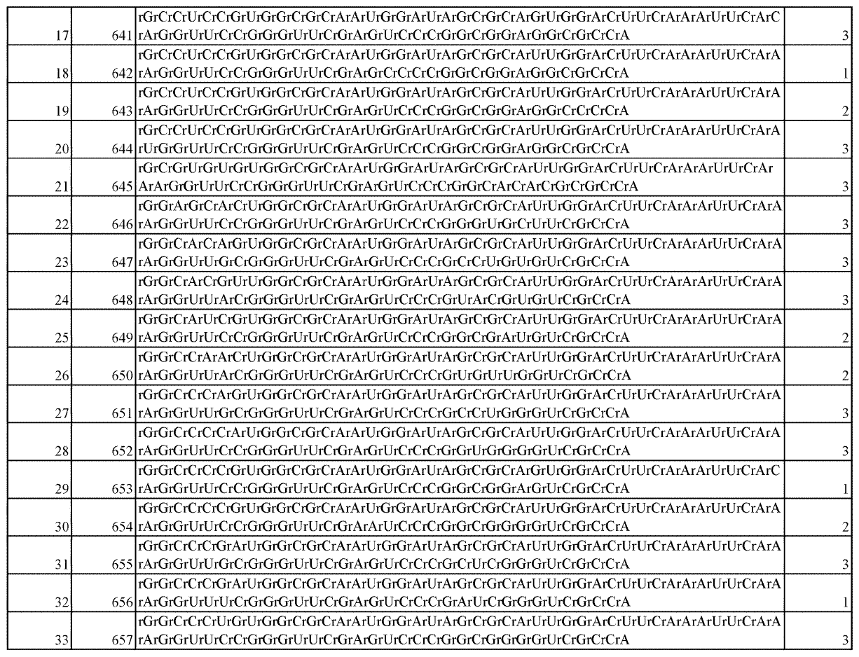

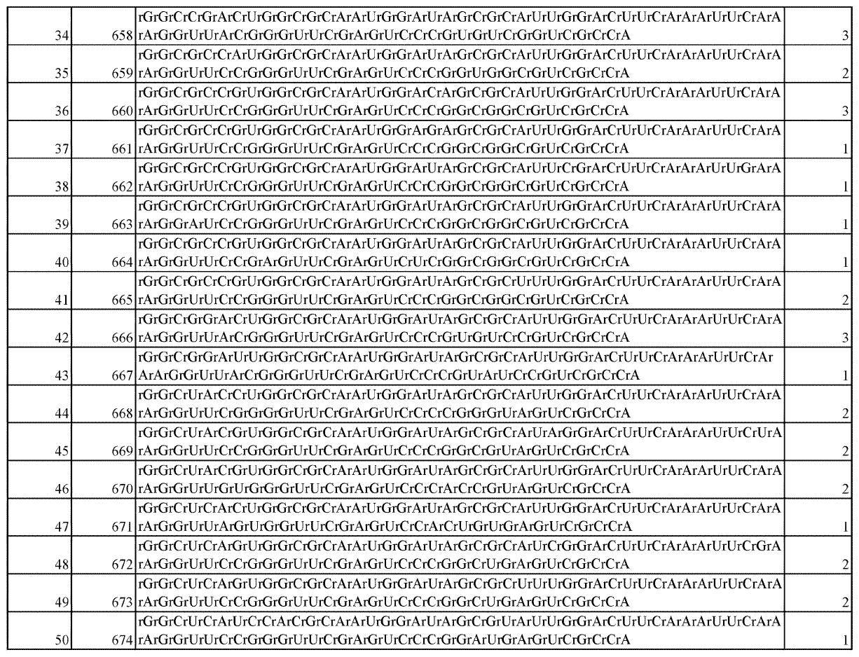

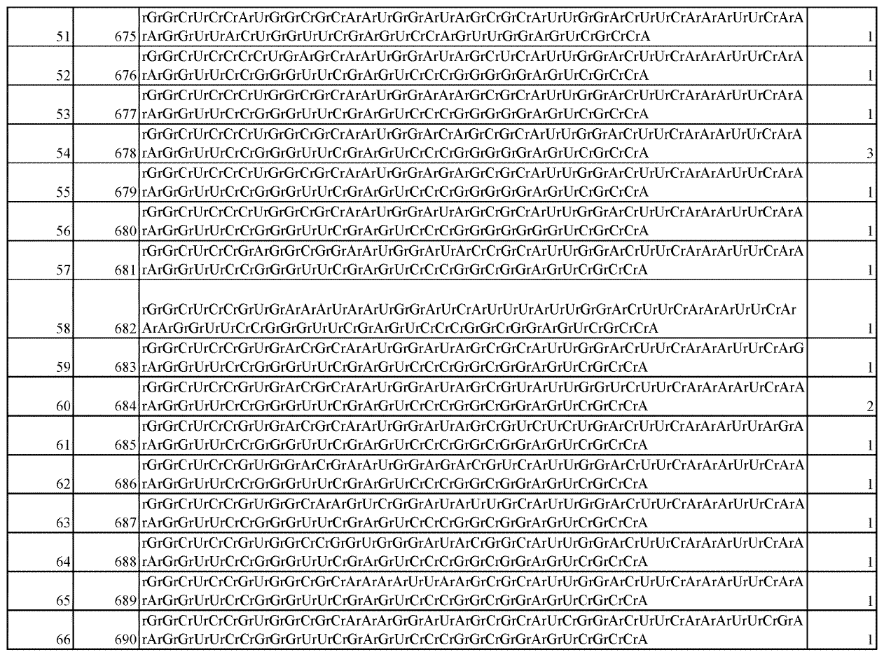

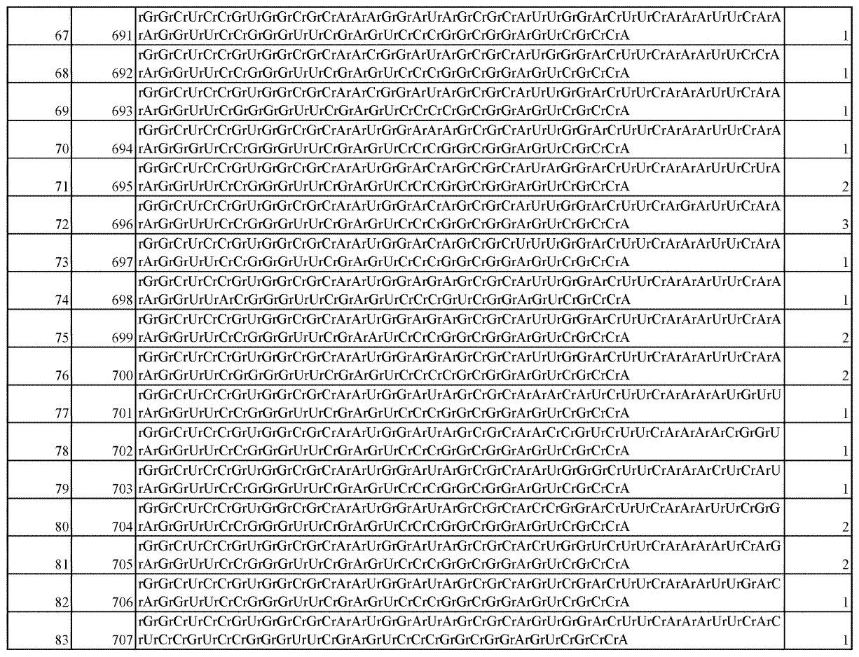

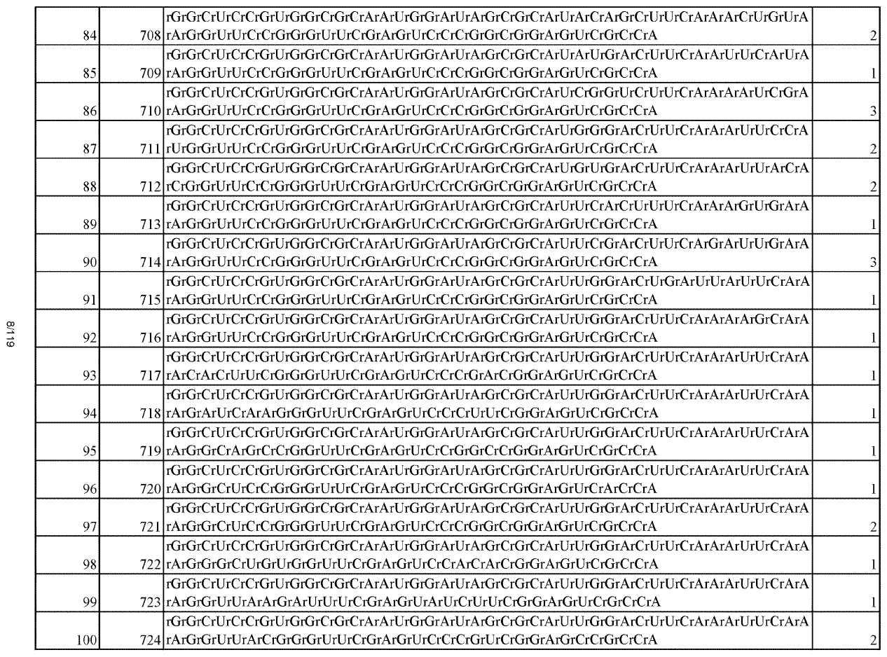

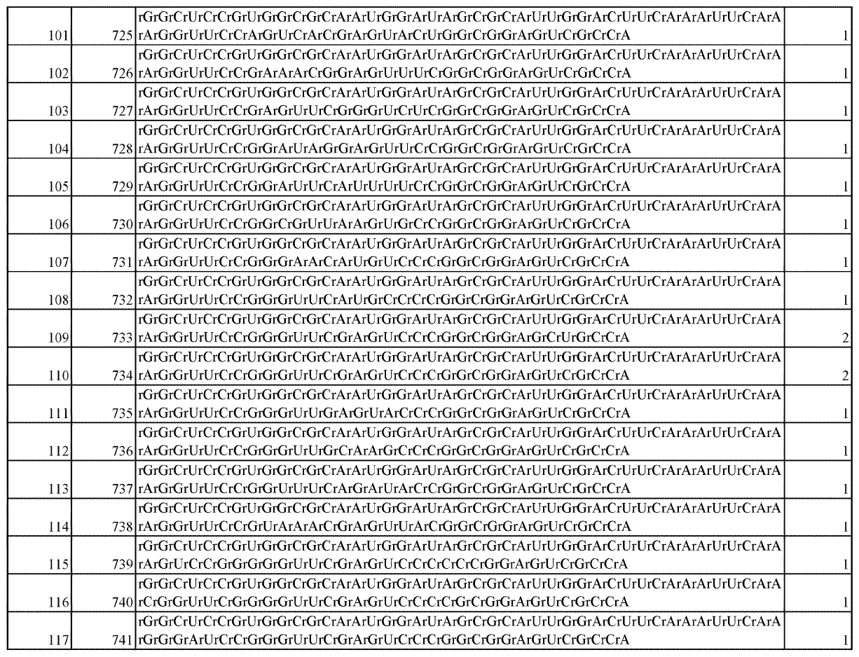

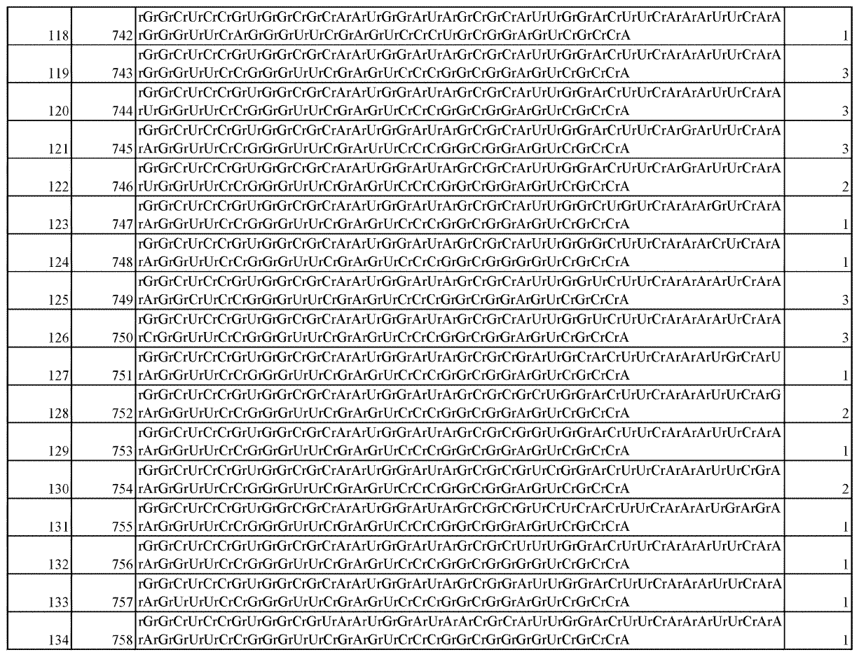

In an embodiment, a TREM may comprise a nucleotide sequence scaffold, e.g., any one of Scaffold Nos: 1, 2, 3, 4, 5, 6, 7, 8, 9, 10, 11, and 12. In an embodiment, a TREM may comprise non-naturally occurring modifications, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or more non-naturally occurring modifications. In an embodiment, a TREM may comprise a nucleotide sequence scaffold, e.g., any one of Scaffold Nos: 1, 2, 3, 4, 5, 6, 7, 8, 9, 10, 11, and 12, and non-naturally occurring modifications, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or more non-naturally occurring modifications. In an embodiment, a functional parameter of a TREM may be modulated by the sequence scaffold. In an embodiment, a functional parameter of a TREM may be modulated by the number of non-naturally occurring modifications. In an embodiment, a functional parameter of a TREM may be modulated by both the sequence scaffold and the number of non-naturally occurring modifications. In an embodiment, the functional parameter of the TREM that is modulated by the nucleotide sequence scaffold, the number of non-naturally occurring modifications, or both the nucleotide sequence scaffold and the number of non-naturally occurring modifications is the log 2 activity of the TREM relative to mock, e.g., log 2 activity of the TREM as provided in FIG. 2. In an embodiment, the log 2 activity of a TREM can be modulated by zero non-naturally occurring modifications to be between about 60 and about 256. In an embodiment, the log 2 activity of a TREM can be modulated by four non-naturally occurring modifications to be between about 128 and about 800. In an embodiment, the log 2 activity of a TREM can be modulated by five non-naturally occurring modifications to be between about 256 and about 600. In an embodiment, the log 2 activity of a TREM can be modulated by six non-naturally occurring modifications to be between about 200 and about 512. In an embodiment, the log 2 activity of a TREM can be modulated by eight non-naturally occurring modifications to be between about 150 and about 700. In an embodiment, the log 2 activity of a TREM can be modulated by ten non-naturally occurring modifications to be between about 3 and about 100. In an embodiment, the log 2 activity of a TREM can be modulated by thirteen non-naturally occurring modifications to be between about 3 and about 100. In an embodiment, the log 2 activity of a TREM can be modulated by fourteen non-naturally occurring modifications to be between about 8 and about 400. In an embodiment, the log 2 activity of a TREM can be modulated by fifteen non-naturally occurring modifications to be between about 32 and about 250. In an embodiment, the log 2 activity of a TREM can be modulated by sixteen non-naturally occurring modifications to be between about 8 and about 400. In an embodiment, the log 2 activity of a TREM can be modulated by nineteen non-naturally occurring modifications to be between about 60 and about 400. In an embodiment, the log 2 activity of a TREM can be modulated by twenty-one non-naturally occurring modifications to be between about 3 and about 128. In an embodiment, the log 2 activity of a TREM can be modulated by twenty-two non-naturally occurring modifications to be between about 8 and about 256. In an embodiment, the log 2 activity of a TREM can be modulated by twenty-four non-naturally occurring modifications to be between about 12 and about 500. In an embodiment, the log 2 activity of a TREM can be modulated by twenty-five non-naturally occurring modifications to be between about 2 and about 60. In an embodiment, the log 2 activity of a TREM can be modulated by twenty-six non-naturally occurring modifications to be between about 3 and about 55. In an embodiment, the log 2 activity of a TREM can be modulated by twenty-seven non-naturally occurring modifications to be between about 60 and about 500. In an embodiment, the log 2 activity of a TREM can be modulated by twenty-eight non-naturally occurring modifications to be between about 2 and about 100. In an embodiment, the log 2 activity of a TREM can be modulated by twenty-nine non-naturally occurring modifications to be between about 55 and about 600. In an embodiment, the log 2 activity of a TREM can be modulated by thirty non-naturally occurring modifications to be between about 4 and about 128. In an embodiment, the log 2 activity of a TREM can be modulated by thirty-one non-naturally occurring modifications to be between about 1 and about 32. In an embodiment, the log 2 activity of a TREM can be modulated by thirty-two non-naturally occurring modifications to be between about 3 and about 128. In an embodiment, the log 2 activity of a TREM can be modulated by thirty-three non-naturally occurring modifications to be between about 64 and about 512. In an embodiment, the log 2 activity of a TREM can be modulated by thirty-four non-naturally occurring modifications to be between about 32 and about 512. In an embodiment, the log 2 activity of a TREM can be modulated by thirty-five non-naturally occurring modifications to be between about 20 and about 512. In an embodiment, the log 2 activity of a TREM can be modulated by thirty-six non-naturally occurring modifications to be between about 2 and about 512. In an embodiment, the log 2 activity of a TREM can be modulated by thirty-seven non-naturally occurring modifications to be between about 3 and about 512. In an embodiment, the log 2 activity of a TREM can be modulated by forty non-naturally occurring modifications to be between about 100 and about 512. In an embodiment, the log 2 activity of a TREM can be modulated by forty-one non-naturally occurring modifications to be between about 6 and about 256. In an embodiment, the log 2 activity of a TREM can be modulated by Scaffold No: 1 to be between about 1 to about 400. In an embodiment, the log 2 activity of a TREM can be modulated by Scaffold No: 2 to be between about 4 to about 512. In an embodiment, the log 2 activity of a TREM can be modulated by Scaffold No: 3 to be between about 4 to about 512. In an embodiment, the log 2 activity of a TREM can be modulated by Scaffold No: 4 to be between about 3 to about 400. In an embodiment, the log 2 activity of a TREM can be modulated by Scaffold No: 5 to be between about 3 to about 700. In an embodiment, the log 2 activity of a TREM can be modulated by Scaffold No: 6 to be between about 3 to about 256. In an embodiment, the log 2 activity of a TREM can be modulated by Scaffold No: 7 to be between about 3 to about 800. In an embodiment, the log 2 activity of a TREM can be modulated by Scaffold No: 8 to be between about 2 to about 600. In an embodiment, the log 2 activity of a TREM can be modulated by Scaffold No: 9 to be between about 3 to about 256. In an embodiment, the log 2 activity of a TREM can be modulated by Scaffold No: 10 to be between about 3 to about 800. In an embodiment, the log 2 activity of a TREM can be modulated by Scaffold No: 11 to be between about 3 to about 500. In an embodiment, the log 2 activity of a TREM can be modulated by Scaffold No: 12 to be between about 1 to about 256.

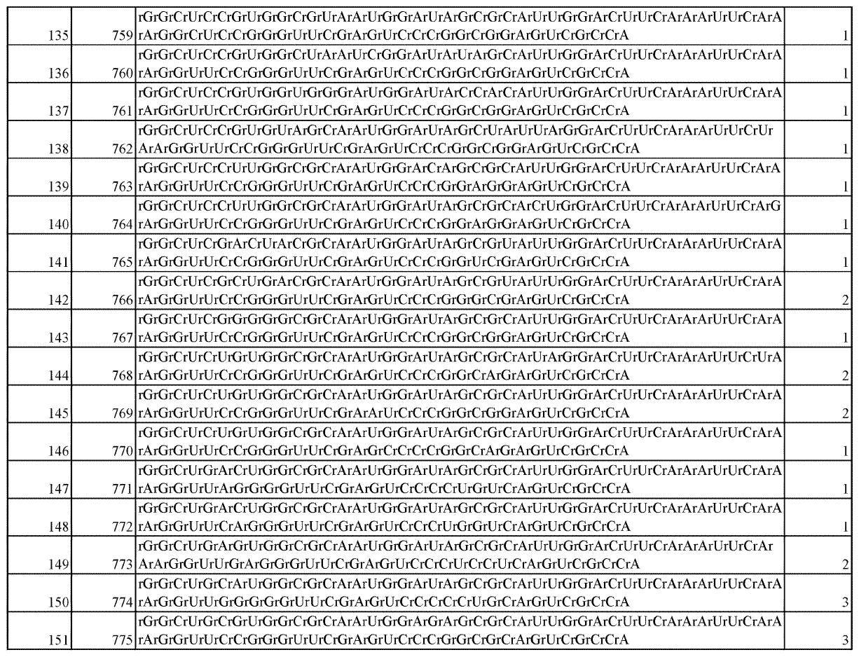

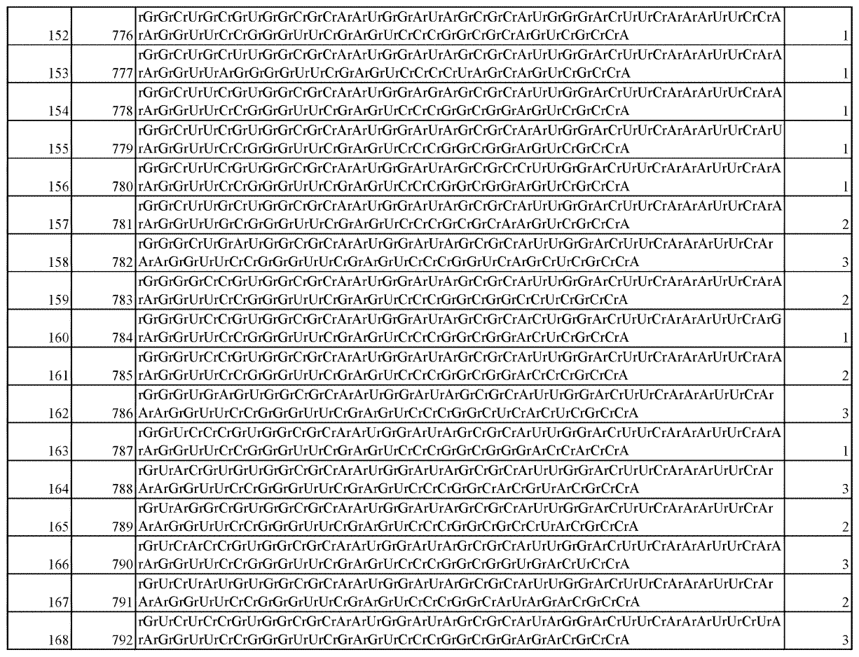

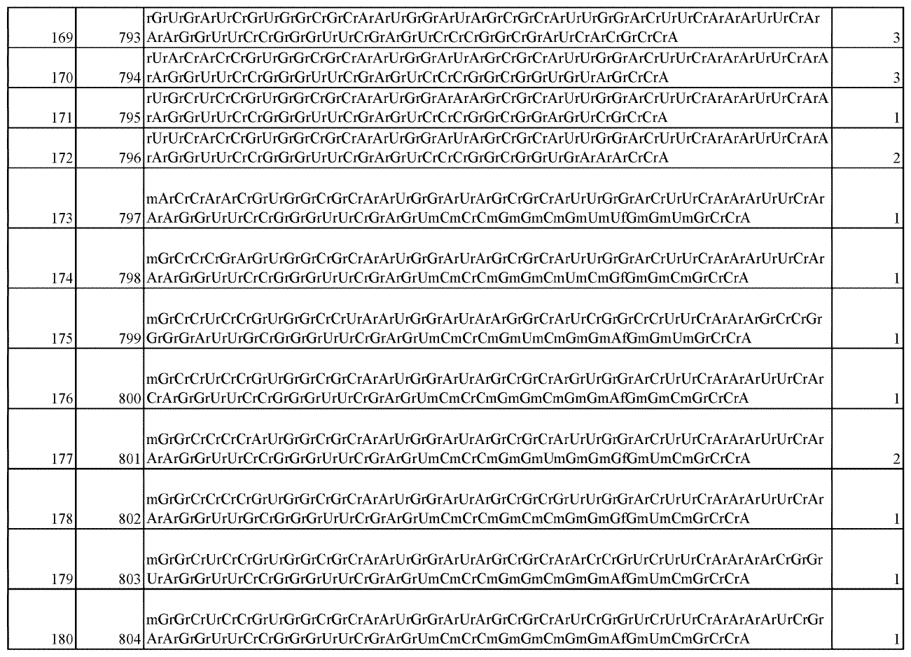

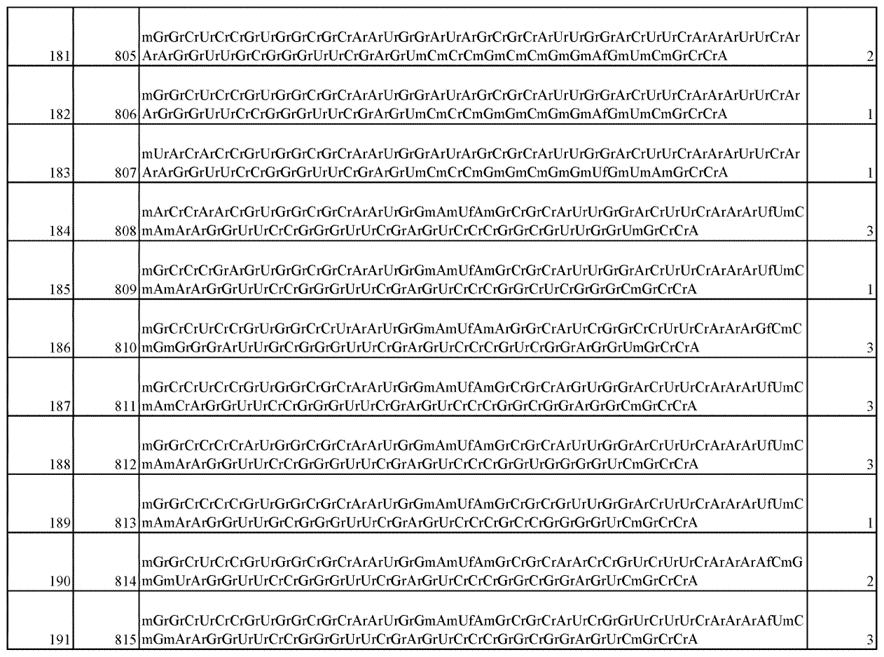

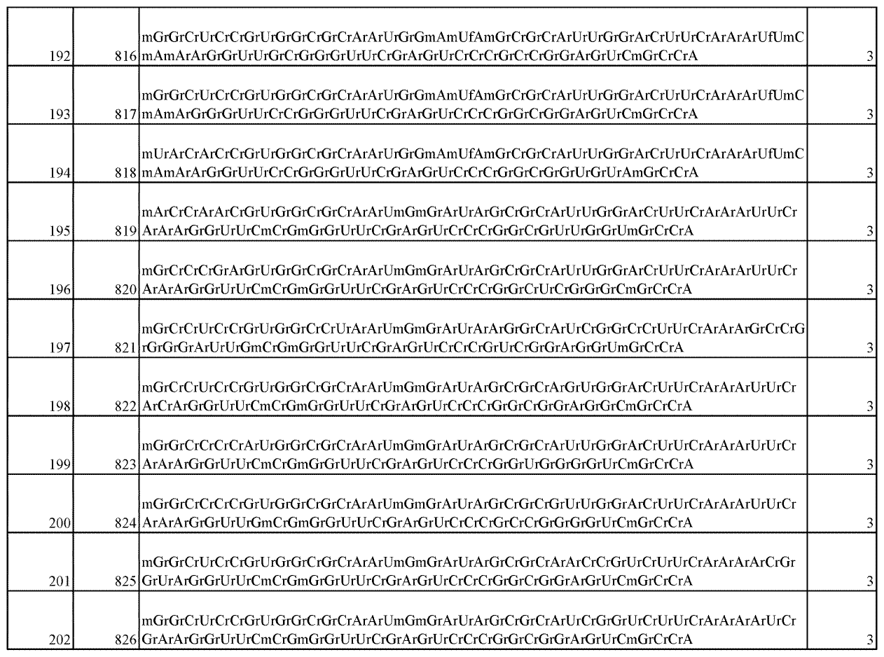

In an embodiment, the TREM is not a TREM provided in FIG. 3. In an embodiment, the TREM does not have the nucleotide sequence of a TREM provided in FIG. 3. In an embodiment, the TREM does not comprise the nucleotide sequence of SEQ ID NO: 743. In an embodiment, the TREM does not comprise the nucleotide sequence of SEQ ID NO: 652. In an embodiment, the TREM does not comprise the nucleotide sequence of SEQ ID NO: 702. In an embodiment, the TREM does not comprise the nucleotide sequence of SEQ ID NO: 627. In an embodiment, the TREM does not comprise the nucleotide sequence of SEQ ID NO: 1150. In an embodiment, the TREM does not comprise the nucleotide sequence of SEQ ID NO: 1151. In an embodiment, the TREM does not comprise the nucleotide sequence of SEQ ID NO: 794. In an embodiment, the TREM does not comprise the nucleotide sequence of SEQ ID NO: 641. In an embodiment, the TREM does not comprise the nucleotide sequence of SEQ ID NO: 710.

In an embodiment, the TREM does not comprise a pattern of non-naturally occurring modifications according to a pattern in Table 6 (e.g., a pattern selected from Pattern Nos. 1-32). In an embodiment, the TREM does not comprise the pattern of non-naturally occurring modifications according to Pattern No. 1 in Table 6. In an embodiment, the TREM does not comprise the pattern of non-naturally occurring modifications according to Pattern No. 2 in Table 6. In an embodiment, the TREM does not comprise the pattern of non-naturally occurring modifications according to Pattern No. 3 in Table 6. In an embodiment, the TREM does not comprise the pattern of non-naturally occurring modifications according to Pattern No. 4 in Table 6. In an embodiment, the TREM does not comprise the pattern of non-naturally occurring modifications according to Pattern No. 5 in Table 6. In an embodiment, the TREM does not comprise the pattern of non-naturally occurring modifications according to Pattern No. 6 in Table 6. In an embodiment, the TREM does not comprise the pattern of non-naturally occurring modifications according to Pattern No. 7 in Table 6. In an embodiment, the TREM does not comprise the pattern of non-naturally occurring modifications according to Pattern No. 8 in Table 6. In an embodiment, the TREM does not comprise the pattern of non-naturally occurring modifications according to Pattern No. 9 in Table 6. In an embodiment, the TREM does not comprise the pattern of non-naturally occurring modifications according to Pattern No. 10 in Table 6. In an embodiment, the TREM does not comprise the pattern of non-naturally occurring modifications according to Pattern No. 11 in Table 6. In an embodiment, the TREM does not comprise the pattern of non-naturally occurring modifications according to Pattern No. 12 in Table 6. In an embodiment, the TREM does not comprise the pattern of non-naturally occurring modifications according to Pattern No. 13 in Table 6. In an embodiment, the TREM does not comprise the pattern of non-naturally occurring modifications according to Pattern No. 14 in Table 6. In an embodiment, the TREM does not comprise the pattern of non-naturally occurring modifications according to Pattern No. 15 in Table 6. In an embodiment, the TREM does not comprise the pattern of non-naturally occurring modifications according to Pattern No. 16 in Table 6. In an embodiment, the TREM does not comprise the pattern of non-naturally occurring modifications according to Pattern No. 17 in Table 6. In an embodiment, the TREM does not comprise the pattern of non-naturally occurring modifications according to Pattern No. 18 in Table 6. In an embodiment, the TREM does not comprise the pattern of non-naturally occurring modifications according to Pattern No. 19 in Table 6. In an embodiment, the TREM does not comprise the pattern of non-naturally occurring modifications according to Pattern No. 20 in Table 6. In an embodiment, the TREM does not comprise the pattern of non-naturally occurring modifications according to Pattern No. 21 in Table 6. In an embodiment, the TREM does not comprise the pattern of non-naturally occurring modifications according to Pattern No. 22 in Table 6. In an embodiment, the TREM does not comprise the pattern of non-naturally occurring modifications according to Pattern No. 23 in Table 6. In an embodiment, the TREM does not comprise the pattern of non-naturally occurring modifications according to Pattern No. 24 in Table 6. In an embodiment, the TREM does not comprise the pattern of non-naturally occurring modifications according to Pattern No. 25 in Table 6. In an embodiment, the TREM does not comprise the pattern of non-naturally occurring modifications according to Pattern No. 26 in Table 6. In an embodiment, the TREM does not comprise the pattern of non-naturally occurring modifications according to Pattern No. 27 in Table 6. In an embodiment, the TREM does not comprise the pattern of non-naturally occurring modifications according to Pattern No. 28 in Table 6. In an embodiment, the TREM does not comprise the pattern of non-naturally occurring modifications according to Pattern No. 29 in Table 6. In an embodiment, the TREM does not comprise the pattern of non-naturally occurring modifications according to Pattern No. 30 in Table 6. In an embodiment, the TREM does not comprise the pattern of non-naturally occurring modifications according to Pattern No. 31 in Table 6. In an embodiment, the TREM does not comprise the pattern of non-naturally occurring modifications according to Pattern No. 32 in Table 6.

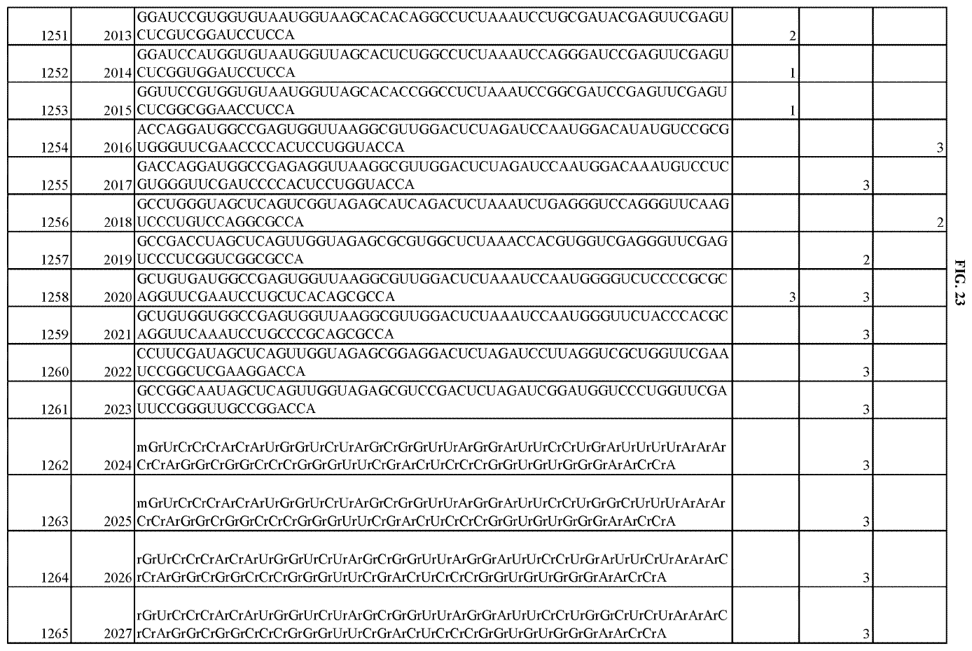

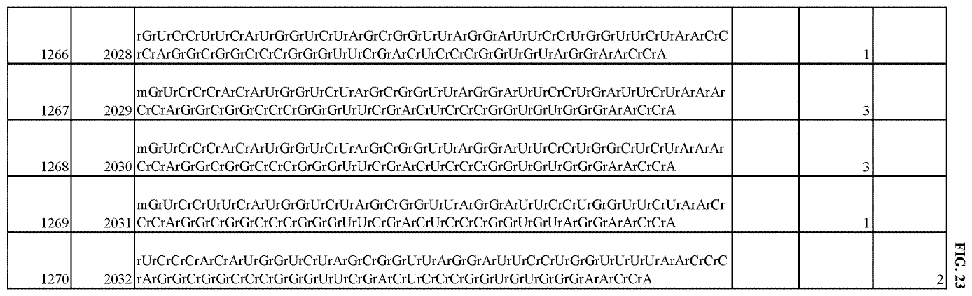

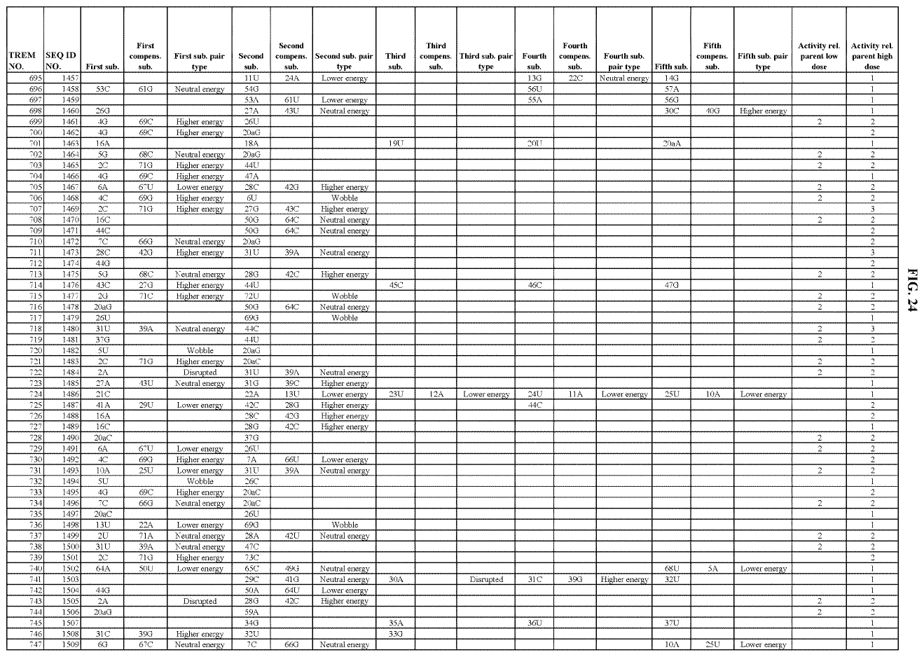

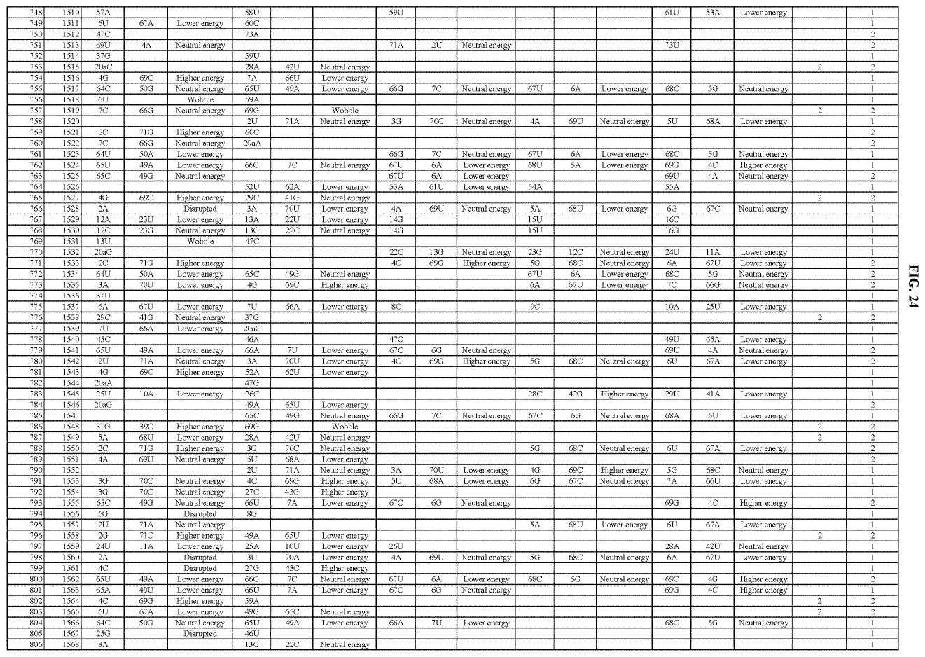

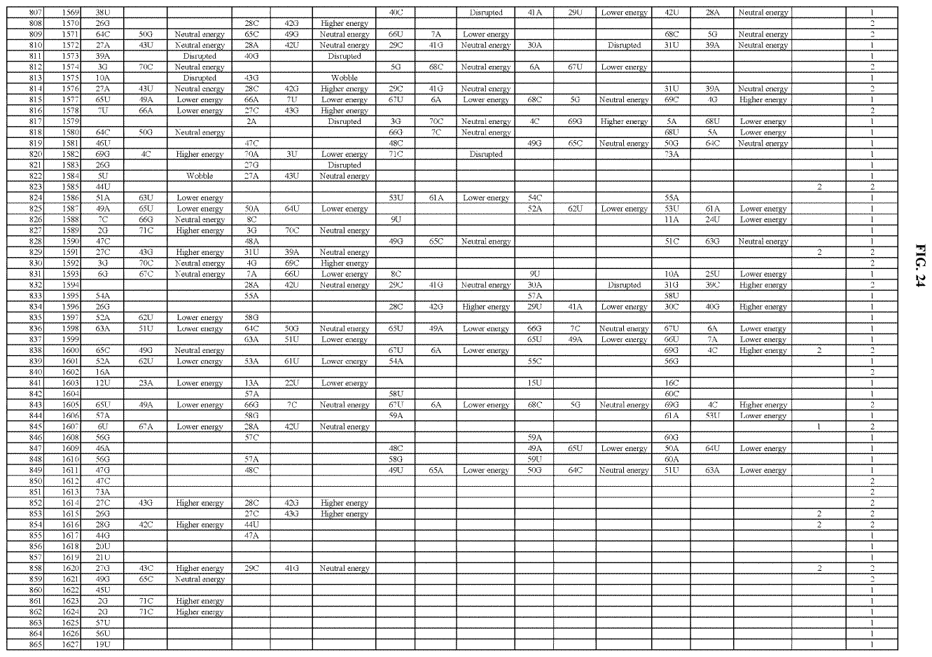

In an embodiment, a TREM comprises a nucleotide sequence, e.g., any one of the sequences provided in FIG. 3 or 23. In an embodiment, a functional parameter of a TREM is modulated by a nucleotide substitution, e.g., a nucleotide substitution relative to a reference TREM sequence. In an embodiment, the functional parameter of the TREM that is modulated by a nucleotide substitution is the premature termination codon (PTC) read-through activity of the TREM, e.g., the ability of a TREM to decode a premature termination codon and continue translation within the coding sequence of a protein. For example, a nucleotide substitution may modulate the relative abundance of a TREM in a pool of hits resulting from a screen of a library of TREMs comprising nucleotide substitutions for PTC readthrough activity. In an embodiment, a nucleotide substitution at a given position in a TREM sequence increases the hit abundance of TREMs comprising said nucleotide substitution relative to a TREM library composition.

In an embodiment, a TREM comprises a nucleotide substitution in a particular region of the TREM. In an embodiment, the nucleotide substitution in a particular region of a TREM modulates a functional parameter of the TREM, e.g., the activity or stability of the TREM. In an embodiment, a nucleotide substitution in a particular region of a TREM modulates activity, e.g., PTC read-through activity, of a TREM. For example, a nucleotide substitution in a particular region of a TREM may increase activity of the TREM and a nucleotide substitution in a second region of a TREM may decrease activity of the TREM. In an embodiment, the activity that is modulated by the nucleotide substitution is the PTC read-through activity of the TREM, e.g., the ability of the TREM to support translation of a premature termination codon (PTC) in a protein coding sequence. In an embodiment, the PTC read-through activity of a TREM modulates the abundance of the TREM in a pool of hits resulting from a pooled screen of a plurality of TREMs comprising nucleotide substitutions. In an embodiment, a TREM having increased PTC read-through activity has greater abundance, e.g., relative to a reference standard, in a pool of hits resulting from a pooled screen of TREMs comprising nucleotide substitutions. In an embodiment, a TREM having decreased PTC read-through activity has lesser abundance, e.g., relative to a reference standard, in a pool of hits resulting from a pooled screen of TREMs comprising nucleotide substitutions. In an embodiment, a nucleotide substitution in a particular region of a TREM modulates the abundance of the TREM in a pool of hits resulting from a pooled screen for PTC read-through activity. In an embodiment, a nucleotide substitution in a particular region of a TREM increases the abundance of the TREM in a pool of hits resulting from a pooled screen. In an embodiment, a nucleotide substitution in the stem, e.g., base-paired region, formed between the [ASt Domain1] and the [ASt Domain2], e.g., the stem formed between positions 1-7 and 66-72, increases the abundance of a TREM in a pool of hits resulting from a pooled screen. In an embodiment, a nucleotide substitution in the stem formed between base pairs at positions 1 and 72, 2 and 71, 3 and 70, 4 and 69, 5 and 68, 6 and 67, and 7 and 66 increases the abundance of a TREM in a pool of hits resulting from a pooled screen, e.g., as shown in FIG. 4A. In an embodiment, a nucleotide substitution in the stem of the [ACH Domain], e.g., the stem formed between positions 27-31 and 39-43, increases the abundance of a TREM in a pool of hits resulting from a pooled screen. In an embodiment, a nucleotide substitution in the stem formed between base pairs at positions 27 and 43, 28 and 42, 29 and 41, and 40, and 31 and 39 increases the abundance of a TREM in a pool of hits resulting from a pooled screen, e.g., as shown in FIG. 4A. In an embodiment, a nucleotide substitution in a particular region of a TREM decreases the abundance of a TREM in a pool of hits resulting from a pooled screen. In an embodiment, a nucleotide substitution in the loop region of the [ACH Domain], e.g., positions 32-38 decreases the abundance of a TREM in a pool of hits resulting from a pooled screen. In an embodiment, a nucleotide substitution in the loop formed at positions 32, 33, 34, 35, 36, 37, and 38 decreases the abundance of a TREM in a pool of hits resulting from a pooled screen, e.g., as shown in FIG. 4A. In an embodiment, a nucleotide substitution in a particular region of a TREM modulates the abundance of various TREM scaffolds, e.g., TREMs specific to various amino acids, e.g., TREMs having the ability to be charged with various amino acids, in a pool of hits resulting from a pooled screen. In an embodiment, a nucleotide substitution in a particular position of a TREM increases the abundance of certain TREM scaffolds in a pool of hits resulting from a pooled screen, e.g., nucleotide substitutions in the base-pair formed between positions 51 and 63. In an embodiment, a nucleotide substitution in the base pair formed between positions 51 and 63 increases the abundance of certain TREM scaffolds in a pool of hits resulting from a pooled screen, e.g., as shown in FIG. 4A.

In an embodiment, the abundance of a nucleotide substitution at a particular position in a TREM in a pool of hits resulting from a pooled screen is modulated by the activity of the TREM, e.g., the PTC read-through activity of the TREM. In an embodiment, the normalized abundance of a nucleotide substitution in a pool of hits is about 0 and about 4.5, e.g., as provided in FIG. 4B. In an embodiment, the normalized abundance of a nucleotide substitution in a pool of hits is about 0 and about 3.5. In an embodiment, the normalized abundance of a nucleotide substitution in a pool of hits is about 0 and about 2.5. In an embodiment, the normalized abundance of a nucleotide substitution in a pool of hits is about 0 and about 2.

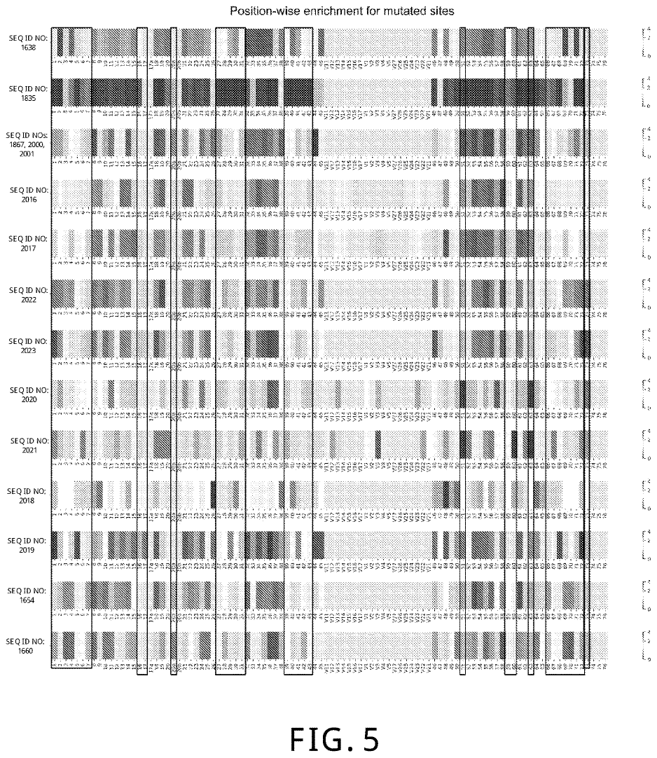

In an embodiment, the enrichment, e.g., abundance, of a nucleotide substitution at a particular position in a TREM in a pool of hits resulting from a pooled screen is modulated by the activity of the TREM, e.g., the PTC read-through activity of the TREM. In an embodiment, a nucleotide substitution at a particular position in a TREM is enriched in a pool of hits resulting from a pooled screen, e.g., is more abundant in the pool of hits than the starting pool composition. In an embodiment, a nucleotide substitution at a particular position in a TREM is depleted in a pool of hits resulting from a pooled screen, e.g., is more abundant in the pool of hits than the starting pool composition. In an embodiment, a nucleotide substitution at a particular position in a TREM is enriched to be between about 0 and about 4, e.g., as provided in FIG. 5. In an embodiment, a nucleotide substitution at any of positions 1, 2, 3, 4, 5, 6, 7, 16, 17, 20a, 27, 28, 29, 30, 31, 39, 40, 41, 42, 43, 51, 59, 60, 63, 66, 67, 68, 69, 70, 71, 72, or 73 increases the abundance of a TREM in a pool of hits resulting from a pooled screen, e.g., as provided in FIG. 5. In an embodiment, a nucleotide substitution at any of positions 1, 2, 3, 4, 5, 6, 7, 16, 17, 20a, 27, 28, 29, 30, 31, 39, 40, 41, 42, 43, 51, 59, 60, 63, 66, 67, 68, 69, 70, 71, 72, or 73 increases premature termination codon (PTC) read-through activity of a TREM, e.g., as provided in FIG. 5. In an embodiment, the activity of a TREM corresponding to a reference TREM, e.g., a reference TREM of any of SEQ ID NOs: 1638, 1835, 1867, 2000, 2001, 2016, 2017, 2022, 2023, 2020, 2021, 2018, 2019, 1654, or 1660, e.g., the PTC read-through activity of a TREM, is increased by a nucleotide substitution at any of positions 1, 2, 3, 4, 5, 6, or 7, e.g., within the [ASt Domain1], e.g., as shown in FIG. 5. In an embodiment, the activity of a TREM corresponding to a reference TREM, e.g., a reference TREM of any of SEQ ID NOs: 1638, 1835, 1867, 2000, 2001, 2016, 2017, 2022, 2023, 2020, 2021, 2018, 2019, 1654, or 1660, e.g., the PTC read-through activity of a TREM, is increased by a nucleotide substitution at any of positions 16 or 17, e.g., within the [DH Domain], e.g., as shown in FIG. 5. In an embodiment, the activity of a TREM corresponding to a reference TREM, e.g., a reference TREM of any of SEQ ID NOs: 1638, 1835, 1867, 2000, 2001, 2016, 2017, 2022, 2023, 2020, 2021, 2018, 2019, 1654, or 1660, e.g., the PTC read-through activity of a TREM, is increased by a nucleotide substitution at position 20a, e.g., within the [DH Domain], e.g., as shown in FIG. 5. In an embodiment, the activity of a TREM corresponding to a reference TREM, e.g., a reference TREM of any of SEQ ID NOs: 1638, 1835, 1867, 2000, 2001, 2016, 2017, 2022, 2023, 2020, 2021, 2018, 2019, 1654, or 1660, e.g., the PTC read-through activity of a TREM, is increased by a nucleotide substitution at any of positions 27, 28, 29, 30, or 31, e.g., within the [ACH Domain], e.g., as shown in FIG. 5. In an embodiment, the activity of a TREM corresponding to a reference TREM, e.g., a reference TREM of any of SEQ ID NOs: 1638, 1835, 1867, 2000, 2001, 2016, 2017, 2022, 2023, 2020, 2021, 2018, 2019, 1654, or 1660, e.g., the PTC read-through activity of a TREM, is increased by a nucleotide substitution at any of positions 39, 40, 41, 42, or 43, e.g., within the [ACH Domain], e.g., as shown in FIG. 5. In an embodiment, the activity of a TREM corresponding to a reference TREM, e.g., a reference TREM of any of SEQ ID NOs: 1638, 1835, 1867, 2000, 2001, 2016, 2017, 2022, 2023, 2020, 2021, 2018, 2019, 1654, or 1660, e.g., the PTC read-through activity of a TREM, is increased by a nucleotide substitution at position 51, e.g., within the [TH Domain], e.g., as shown in FIG. 5. In an embodiment, the activity of a TREM corresponding to a reference TREM, e.g., a reference TREM of any of SEQ ID NOs: 1638, 1835, 1867, 2000, 2001, 2016, 2017, 2022, 2023, 2020, 2021, 2018, 2019, 1654, or 1660, e.g., the PTC read-through activity of a TREM, is increased by a nucleotide substitution at any of positions 59 or 60, e.g., within the [TH Domain], e.g., as shown in FIG. 5. In an embodiment, the activity of a TREM corresponding to a reference TREM, e.g., a reference TREM of any of SEQ ID NOs: 1638, 1835, 1867, 2000, 2001, 2016, 2017, 2022, 2023, 2020, 2021, 2018, 2019, 1654, or 1660, e.g., the PTC read-through activity of a TREM, is increased by a nucleotide substitution at position 63, e.g., within the [TH Domain], e.g., as shown in FIG. 5. In an embodiment, the activity of a TREM corresponding to a reference TREM, e.g., a reference TREM of any of SEQ ID NOs: 1638, 1835, 1867, 2000, 2001, 2016, 2017, 2022, 2023, 2020, 2021, 2018, 2019, 1654, or 1660, e.g., the PTC read-through activity of a TREM, is increased by a nucleotide substitution at any of positions 66, 67, 68, 69, 70, 71, 72, or 73, e.g., within the [ASt Domain2], e.g., as shown in FIG. 5. In an embodiment, the activity of a TREM corresponding to a reference TREM, e.g., a reference TREM of any of SEQ ID NOs: 1638, 1835, 1867, 2000, 2001, 2016, 2017, 2022, 2023, 2020, 2021, 2018, 2019, 1654, or 1660, e.g., the PTC read-through activity of a TREM, is decreased by a nucleotide substitution at any of positions 32, 33, 34, 35, 36, 37, or 38, e.g., within the [ACH Domain], e.g., as shown in FIG. 5.

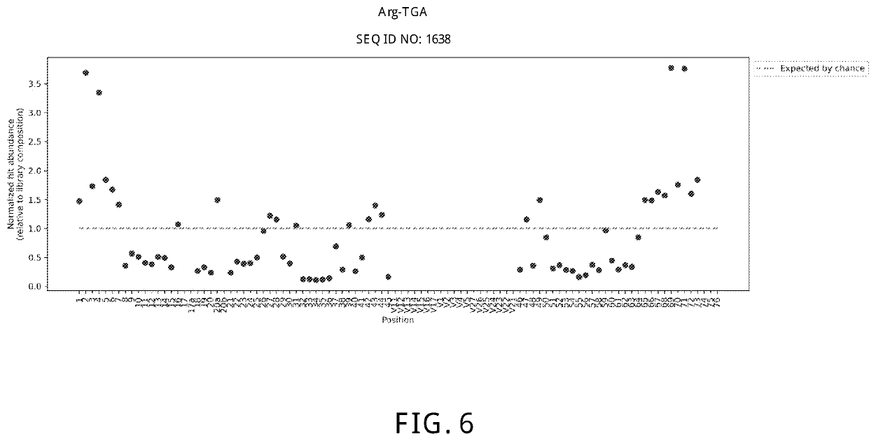

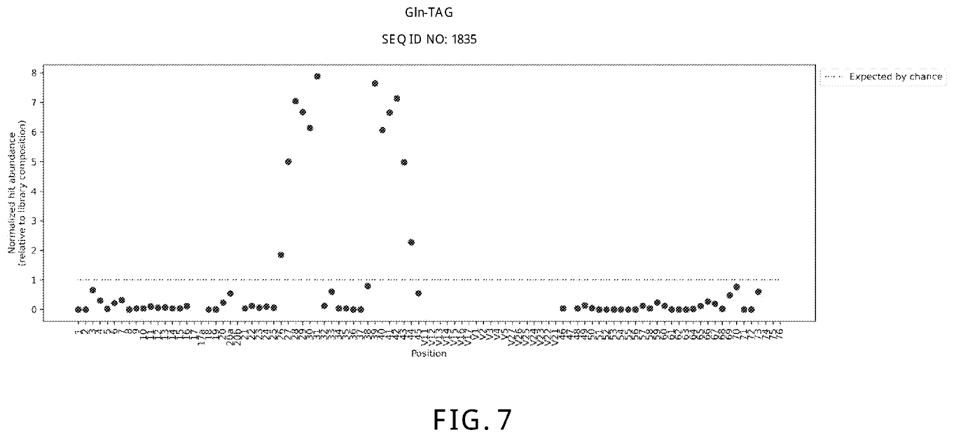

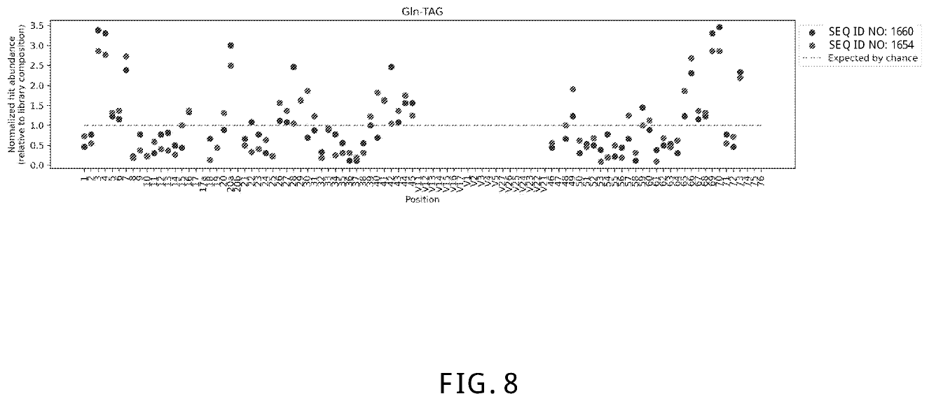

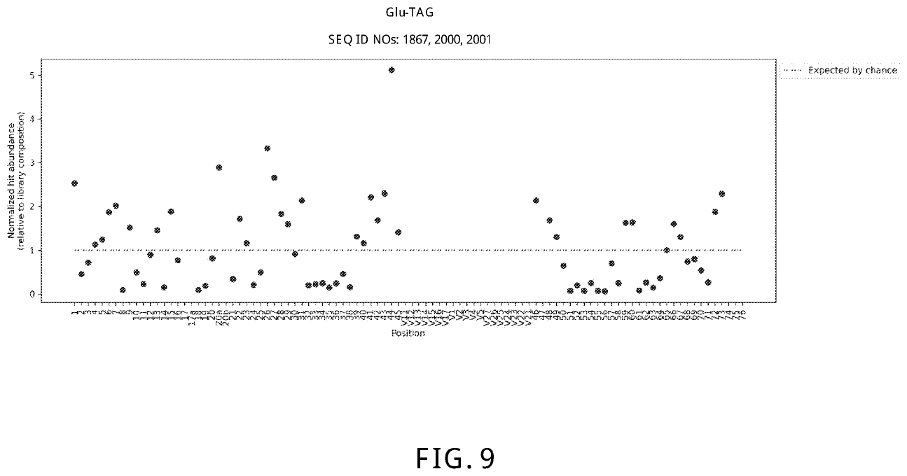

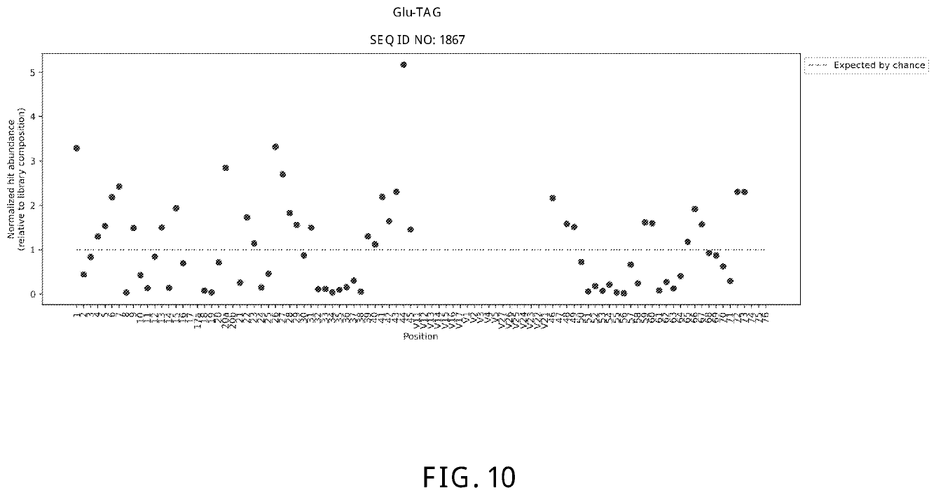

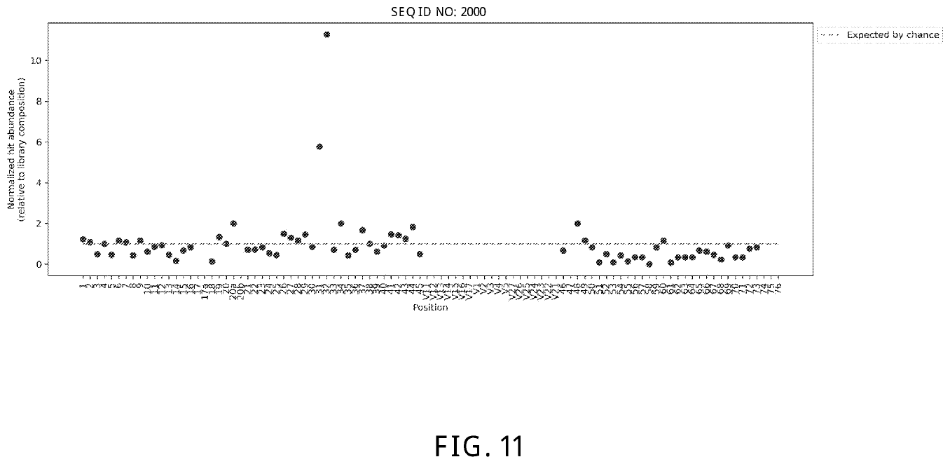

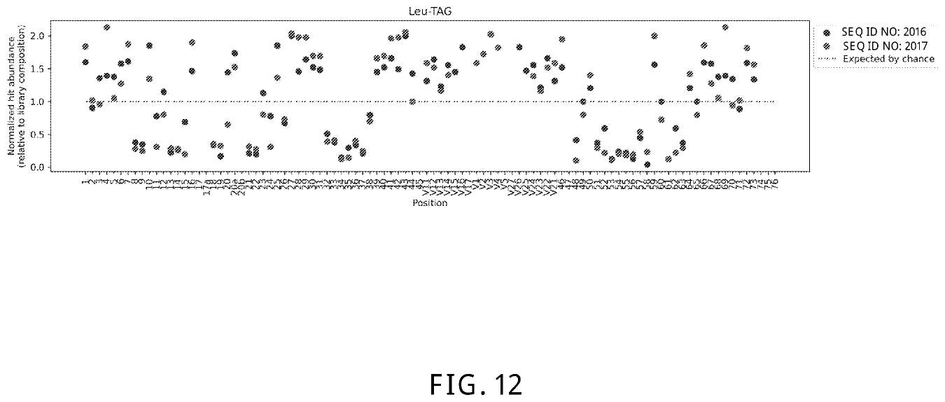

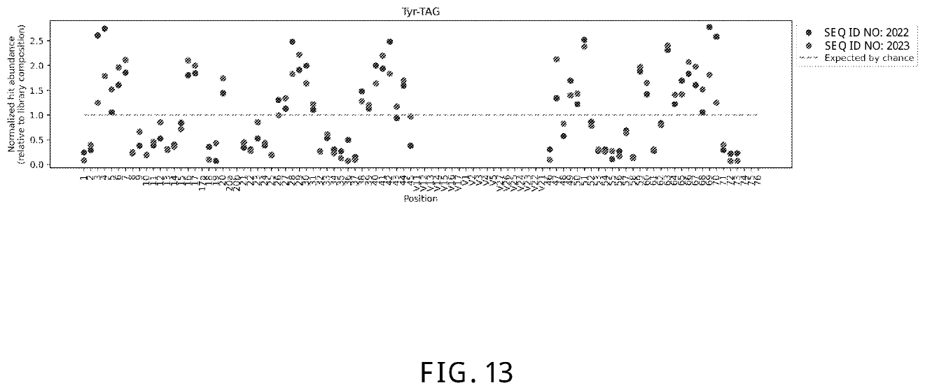

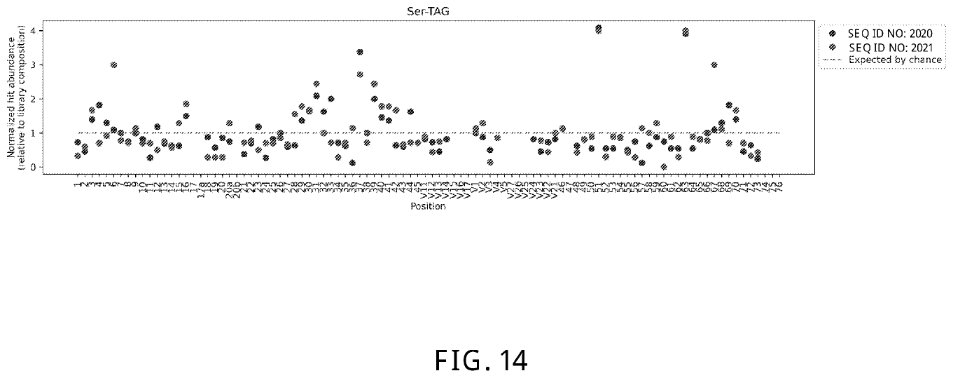

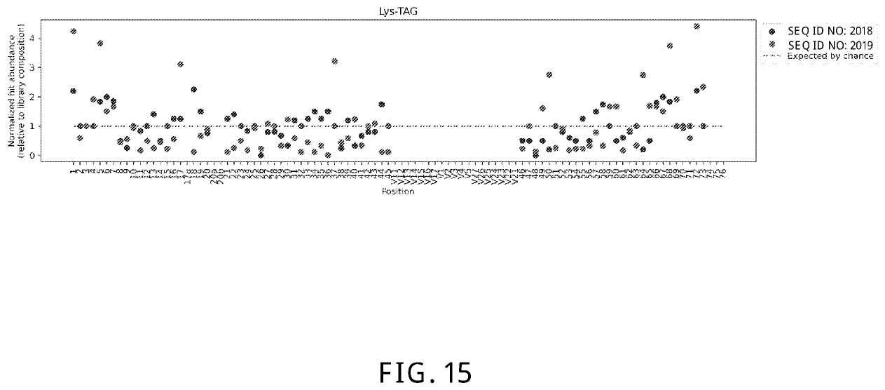

In an embodiment, the abundance of a nucleotide substitution at a particular position in a TREM in a pool of hits resulting from a pooled screen is modulated by the activity of the TREM, e.g., the PTC read-through activity of the TREM. In an embodiment, the normalized abundance of a nucleotide substitution in a pool of hits is about 0 and about 3.6, e.g., as provided in FIG. 6. In an embodiment, the normalized abundance of a nucleotide substitution in a pool of hits is about 0 and about 8, e.g., as provided in FIG. 7. In an embodiment, the normalized abundance of a nucleotide substitution in a pool of hits is about 0 and about 3.5, e.g., as provided in FIG. 8. In an embodiment, the normalized abundance of a nucleotide substitution in a pool of hits is about 0 and about 5, e.g., as provided in FIG. 9. In an embodiment, the normalized abundance of a nucleotide substitution in a pool of hits is about 0 and about 5, e.g., as provided in FIG. 10. In an embodiment, the normalized abundance of a nucleotide substitution in a pool of hits is about 0 and about 12, e.g., as provided in FIG. 11. In an embodiment, the normalized abundance of a nucleotide substitution in a pool of hits is about 0 and about 2.1, e.g., as provided in FIG. 12. In an embodiment, the normalized abundance of a nucleotide substitution in a pool of hits is about 0 and about 2.4, e.g., as provided in FIG. 13. In an embodiment, the normalized abundance of a nucleotide substitution in a pool of hits is about 0 and about 4, e.g., as provided in FIG. 14. In an embodiment, the normalized abundance of a nucleotide substitution in a pool of hits is about 0 and about 4.5, e.g., as provided in FIG. 15.

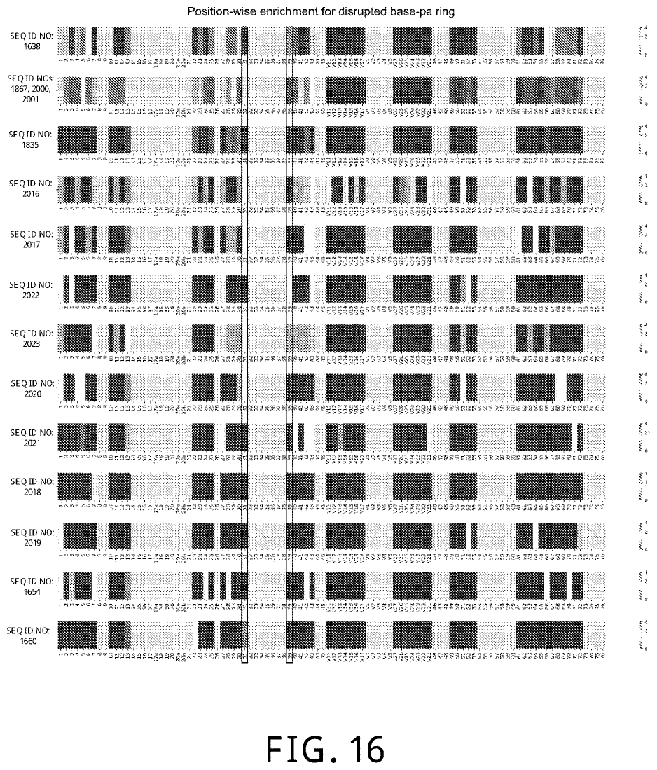

In an embodiment, the enrichment, e.g., abundance, of a nucleotide substitution at a particular position in a TREM in a pool of hits resulting from a pooled screen is modulated by the activity of the TREM, e.g., the PTC read-through activity of the TREM. In an embodiment, the nucleotide substitution disrupts base-pairing at a particular position in a TREM. In an embodiment, a nucleotide substitution that disrupts base-pairing is enriched in a pool of hits resulting from a pooled screen. In an embodiment, a nucleotide substitution that disrupts base-pairing is depleted in a pool of hits resulting from a pooled screen. In an embodiment, a nucleotide substitution that disrupts base-pairing is enriched to be about 0 toa bout 4, e.g., as provided in FIG. 16.

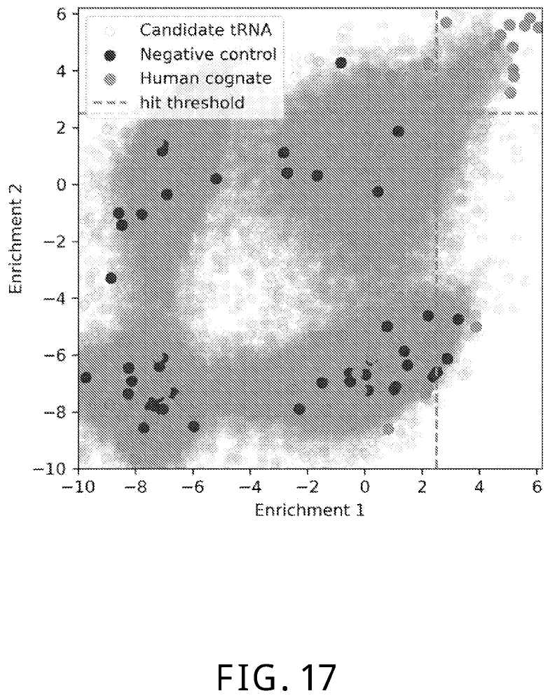

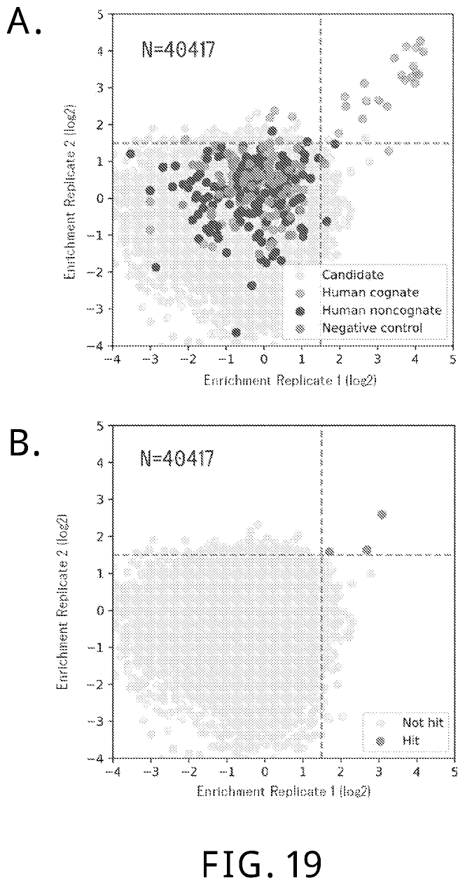

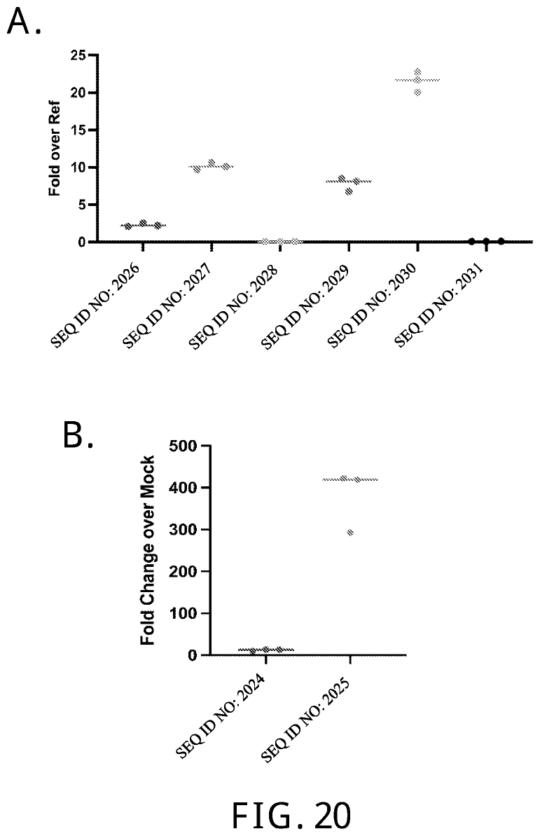

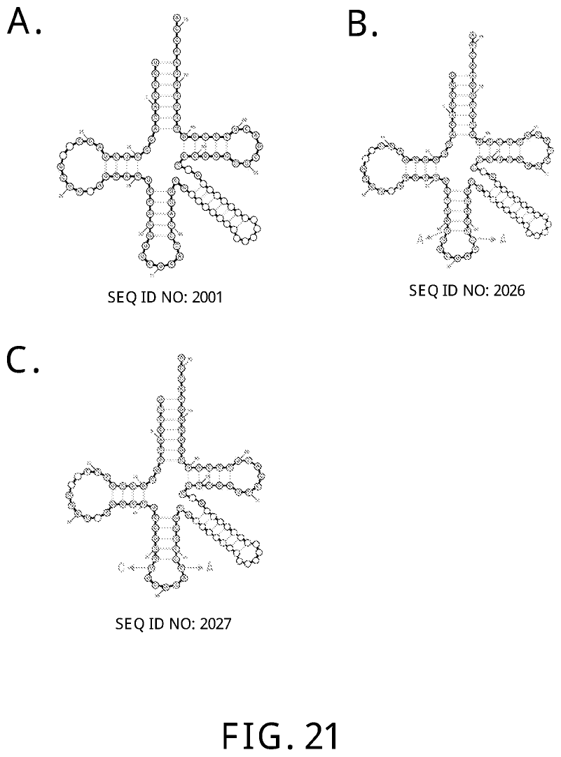

In an embodiment, the enrichment of a TREM comprising a nucleotide substitution in a pool of hits, e.g., a pool of TREMs having increased activity, e.g., increased PTC read-through activity, is reproducible between replicate screens, e.g., as shown in FIGS. 17 and 19A-B.

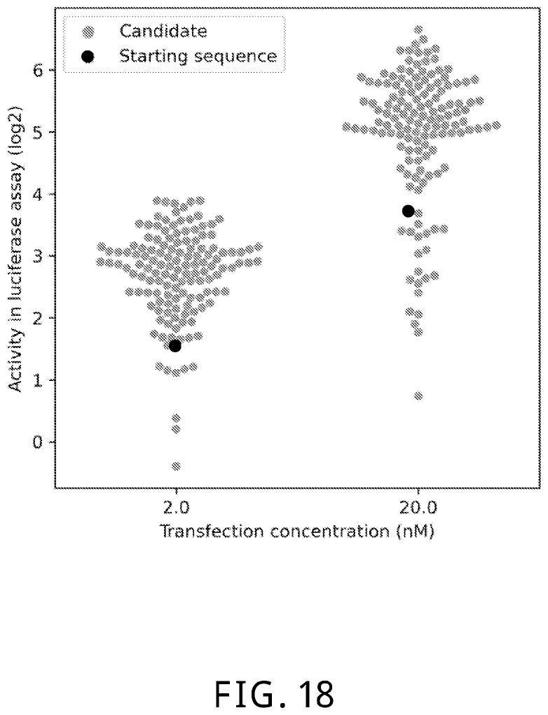

In an embodiment, the log 2 PTC read-through activity of a TREM comprising a nucleotide substitution, e.g., relative to a parent TREM sequence, e.g., starting TREM sequence, is modulated by the nucleotide substitution. In an embodiment, the log 2 PTC read-through activity of a TREM is modulated by a nucleotide substitution to be between about −0.5 to about 6.8, e.g., as provided in FIG. 18. In an embodiment, the log 2 PTC read-through activity of a TREM is modulated by a nucleotide substitution to be between about −0.5 to about 4 at a low TREM dose. In an embodiment, the log 2 PTC read-through activity of a TREM is modulated by a nucleotide substitution to be between about 0.5 to about 6.8 at a high TREM dose.

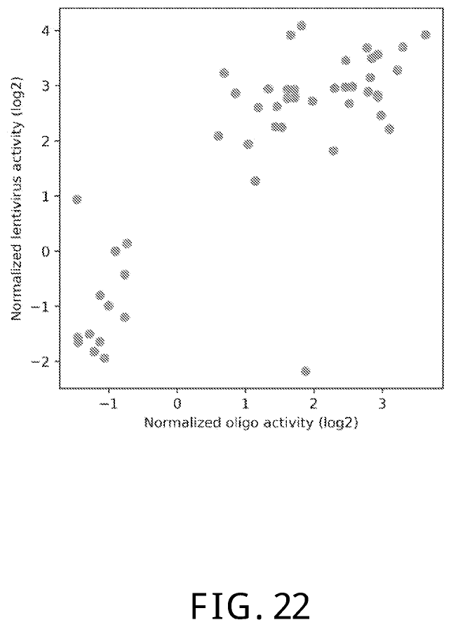

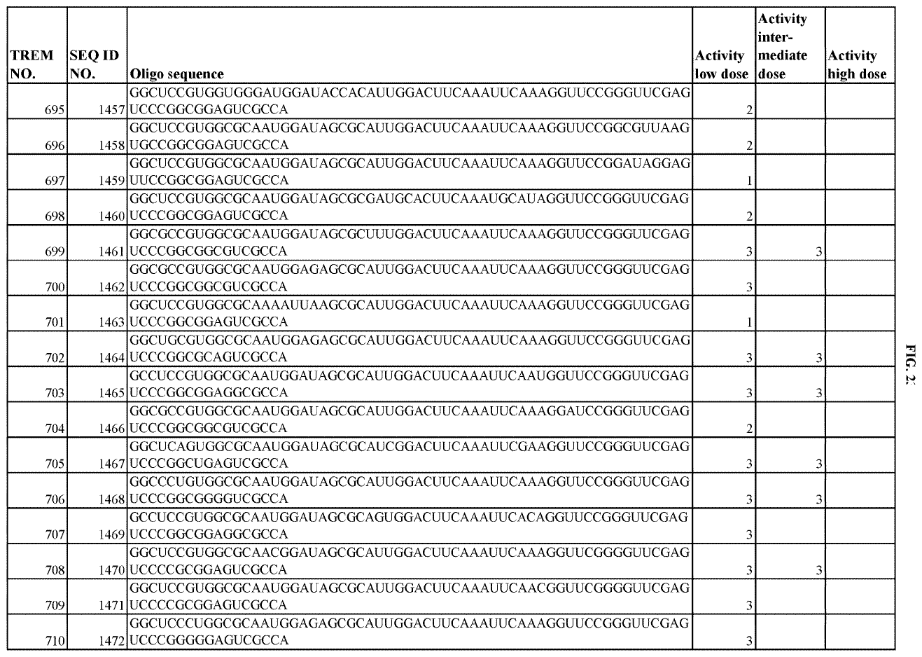

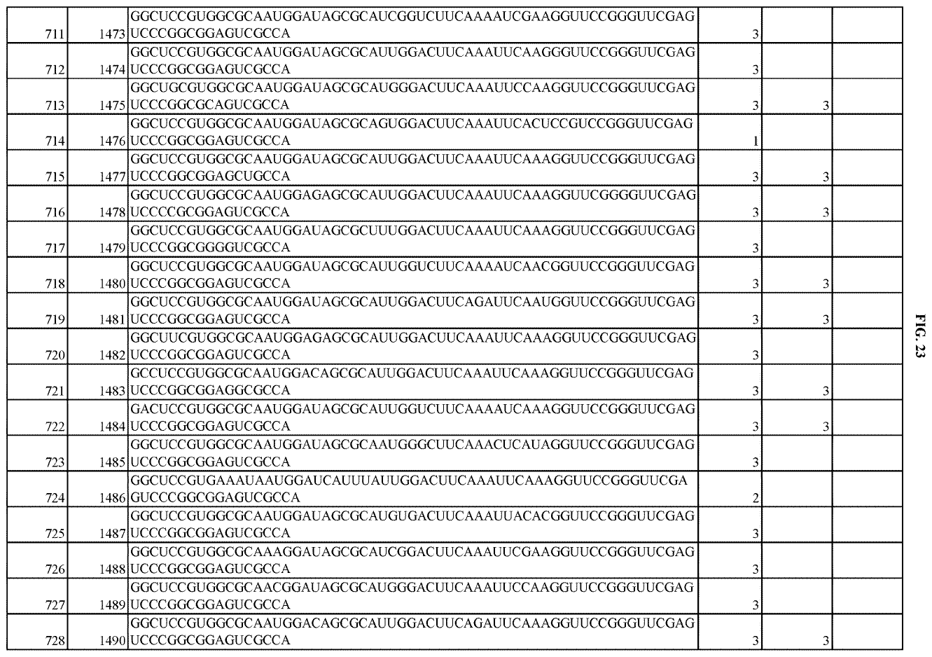

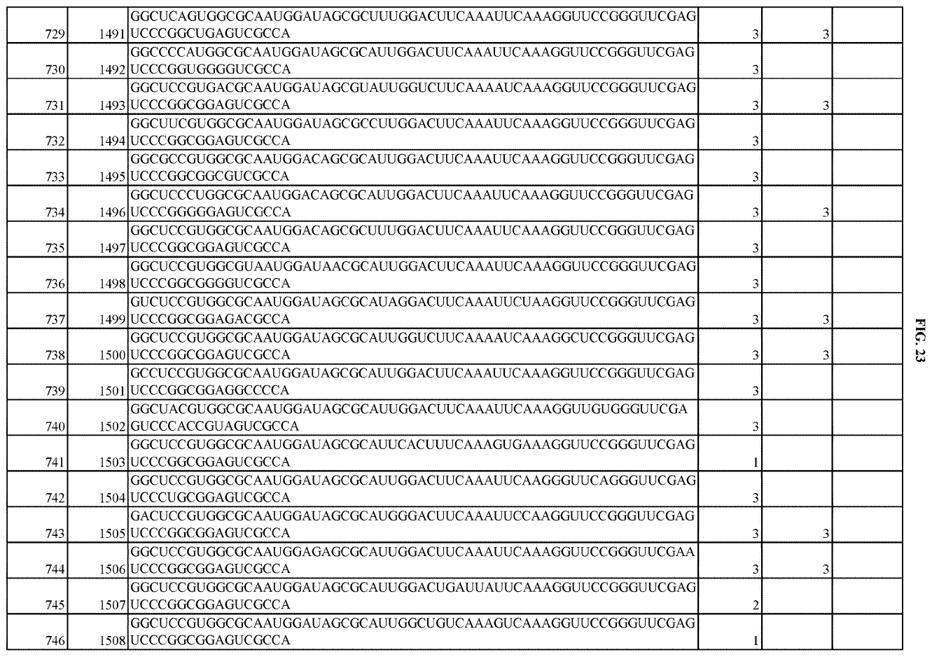

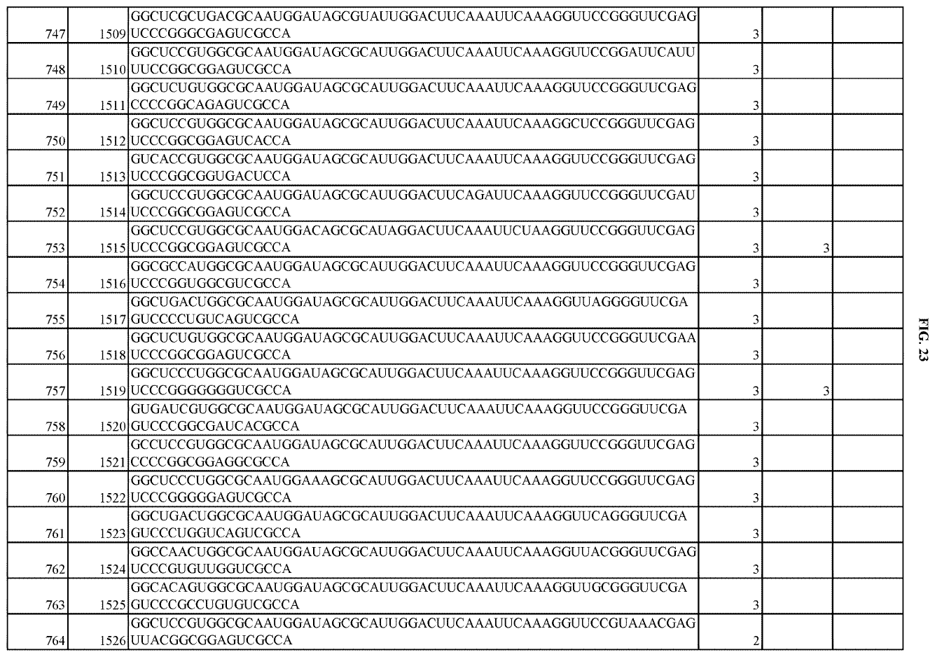

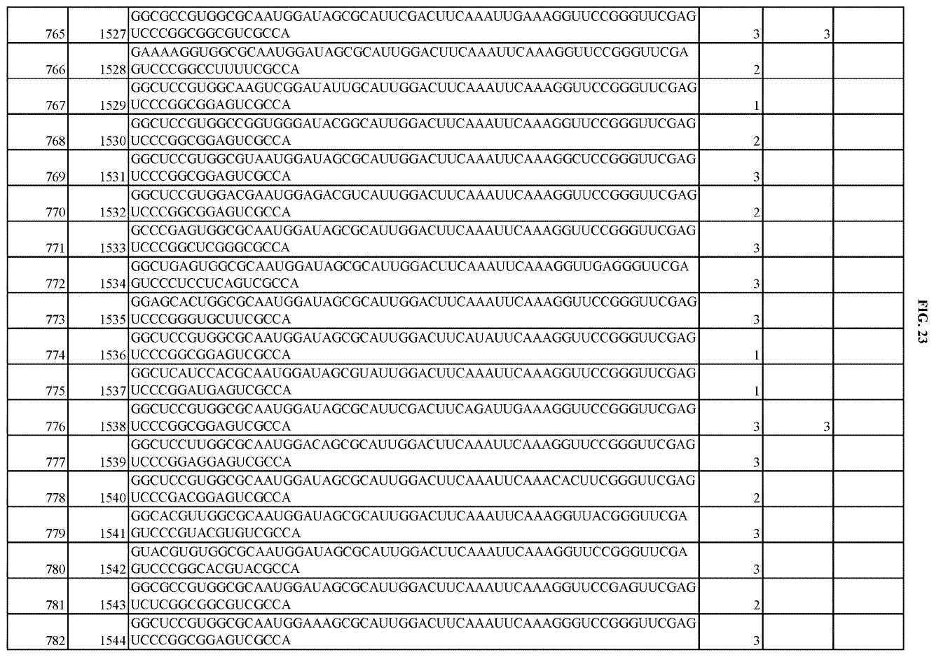

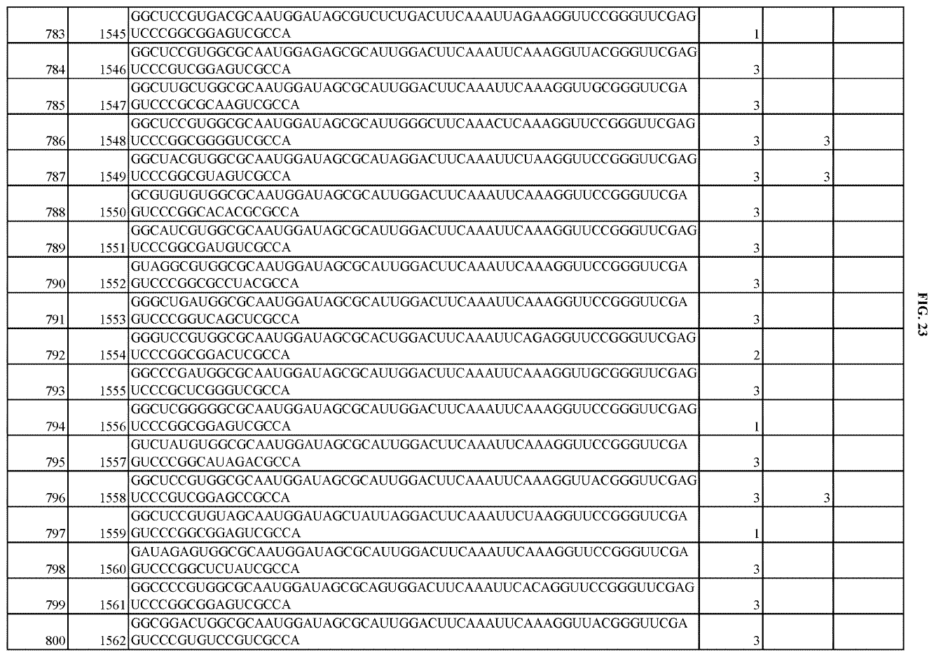

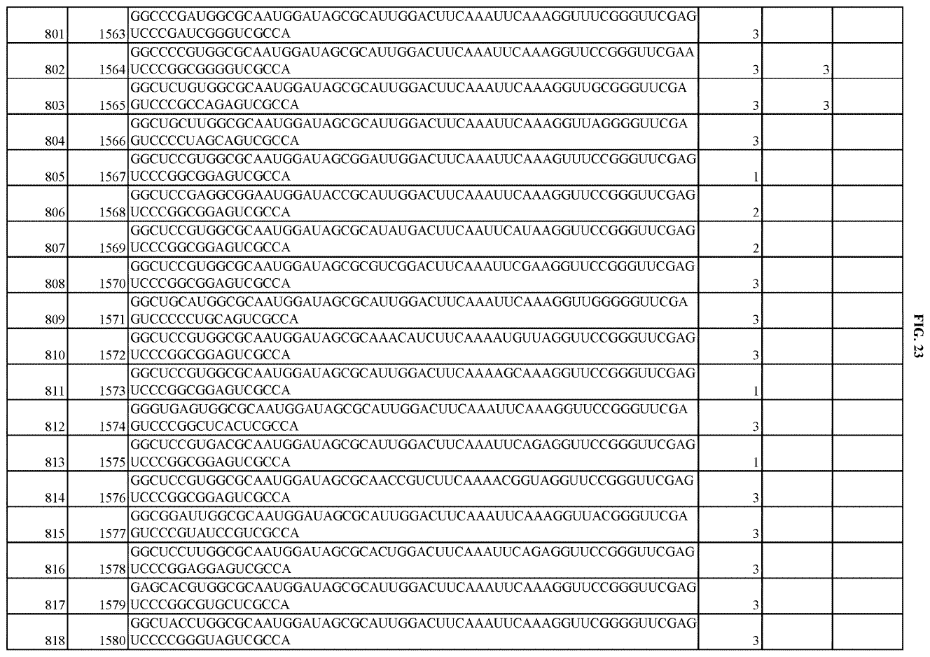

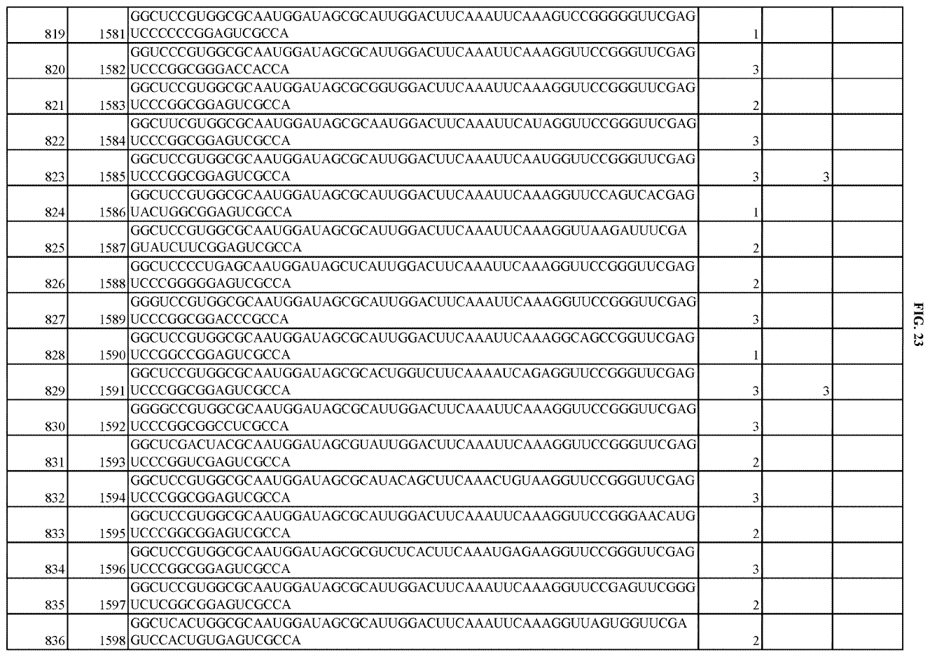

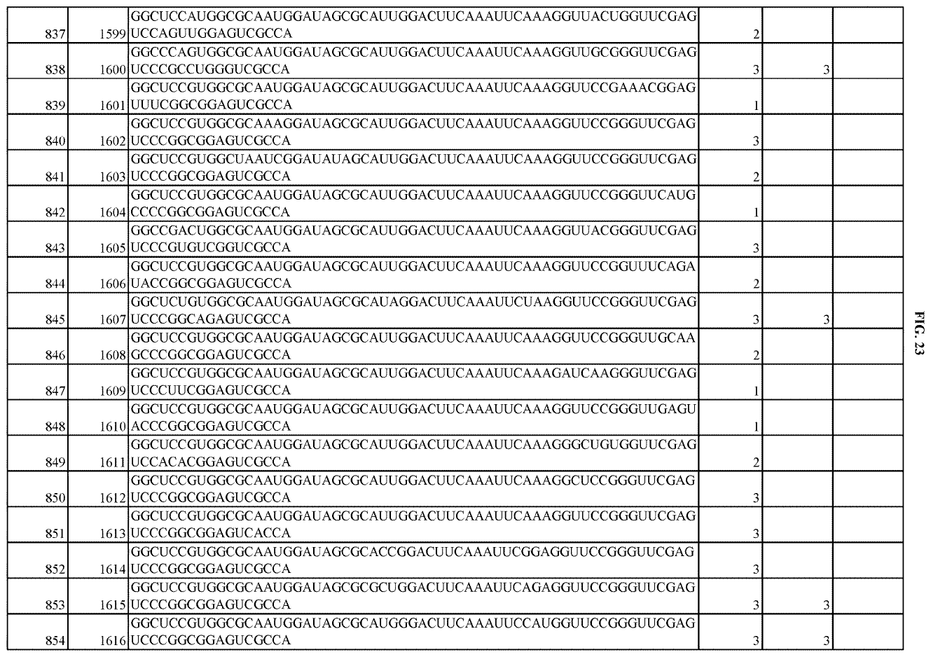

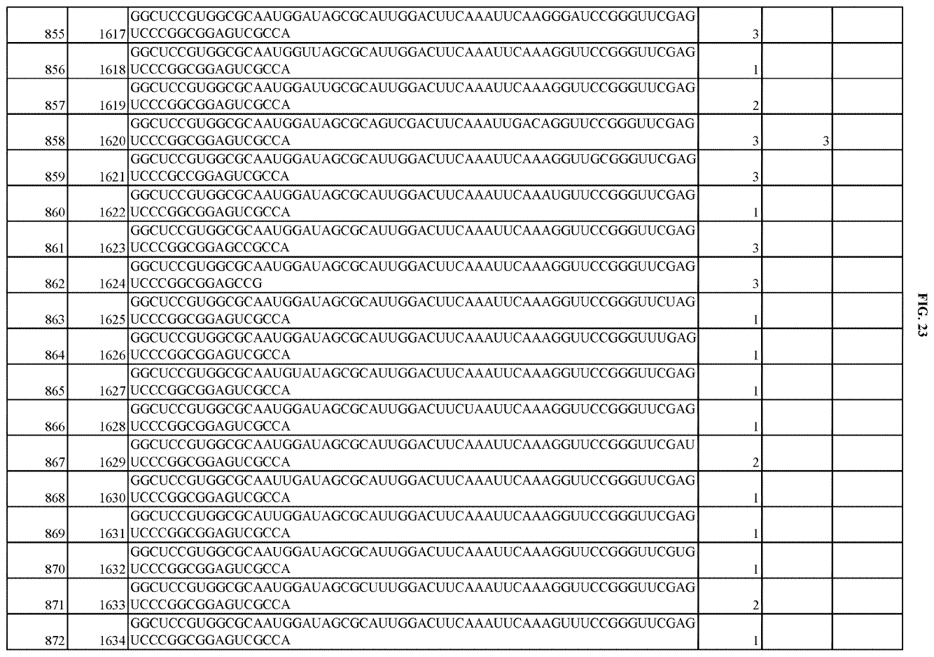

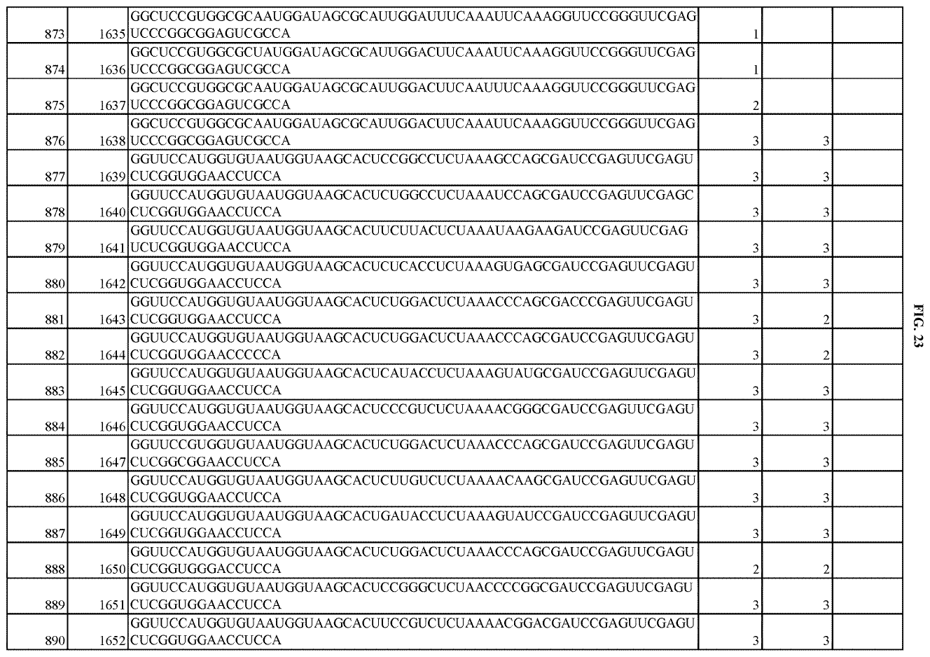

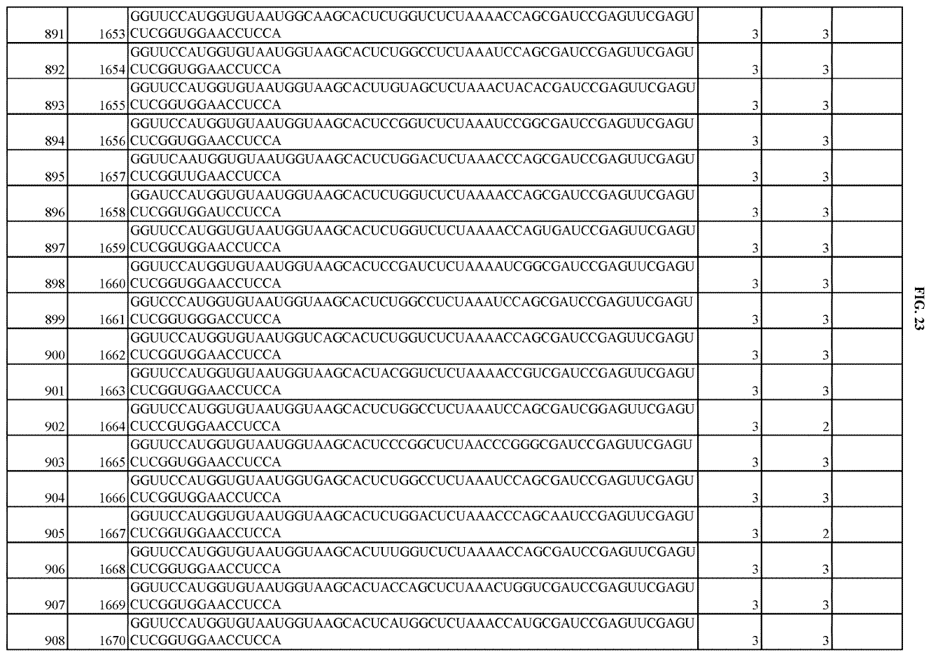

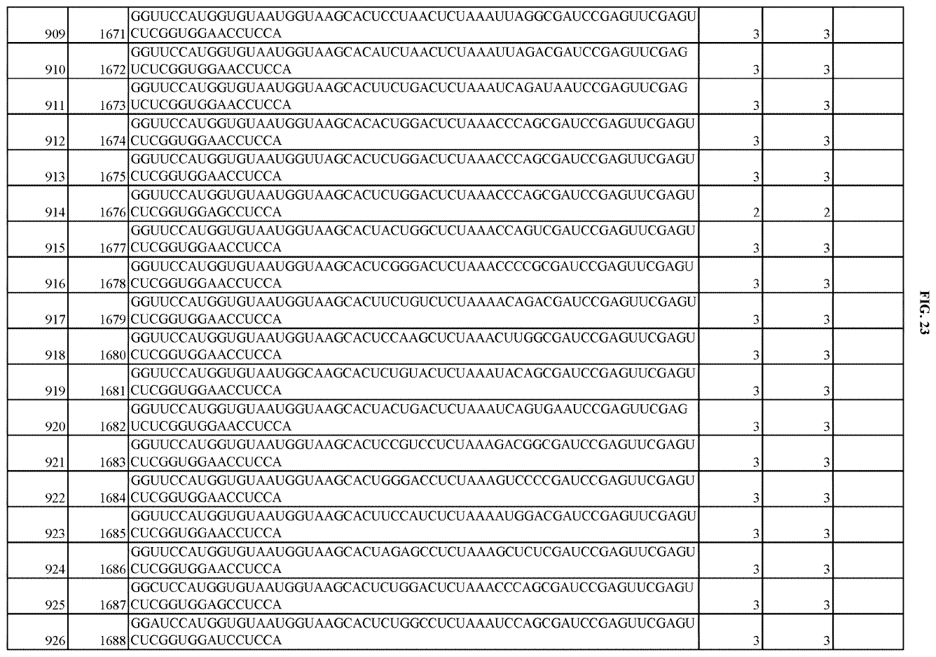

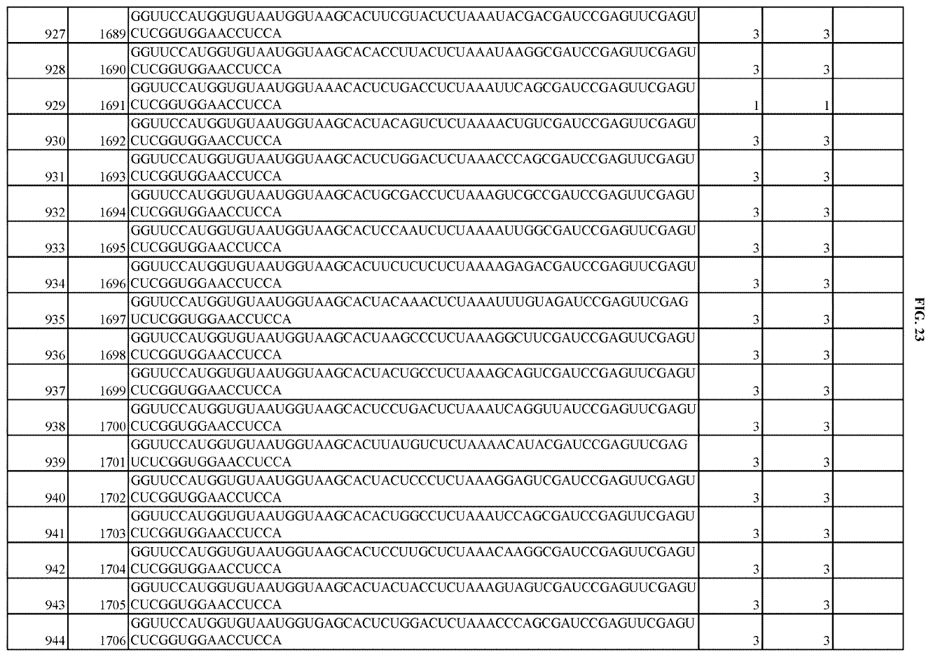

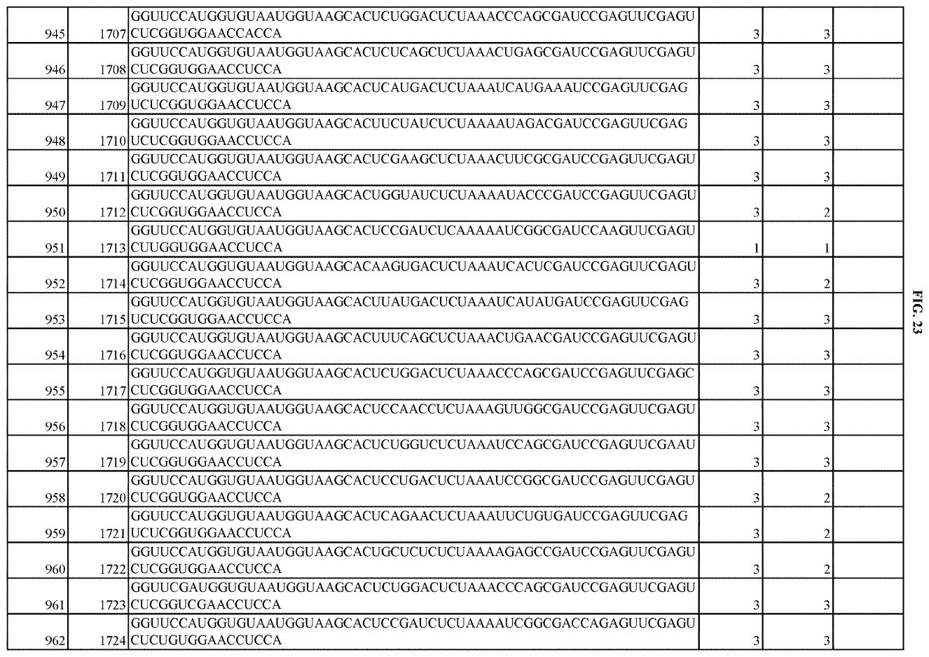

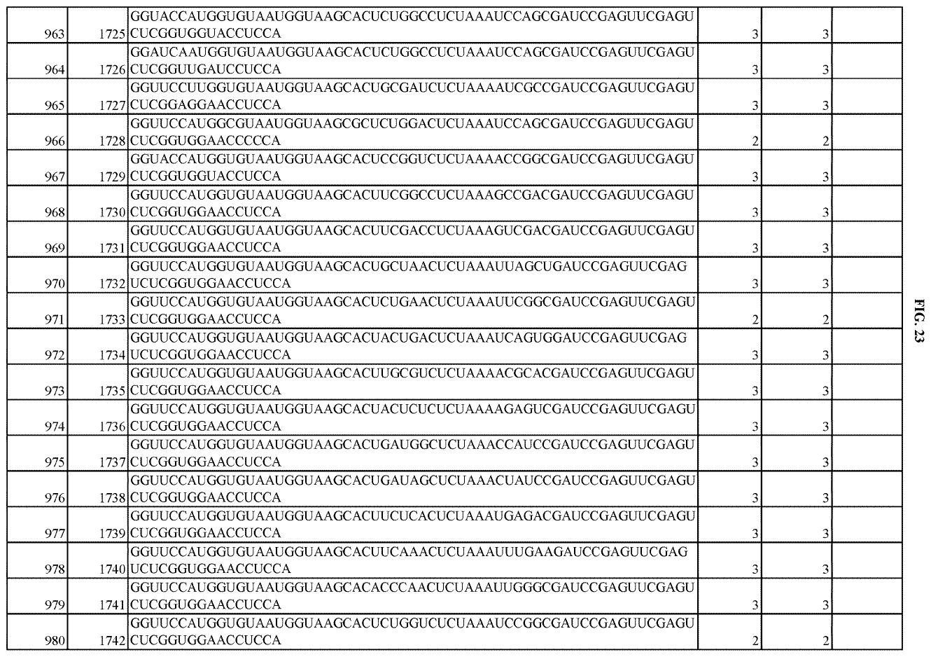

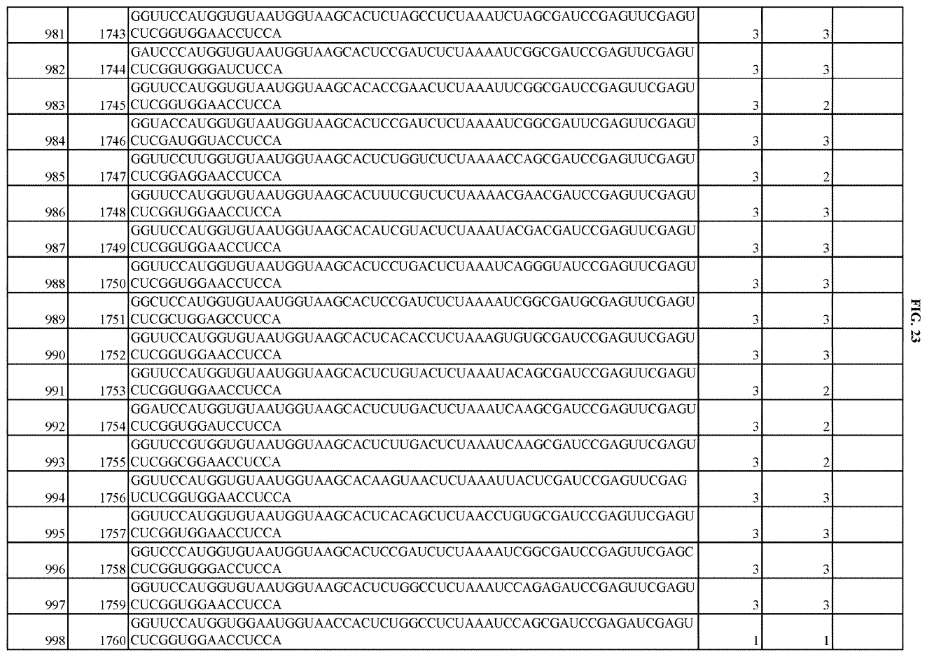

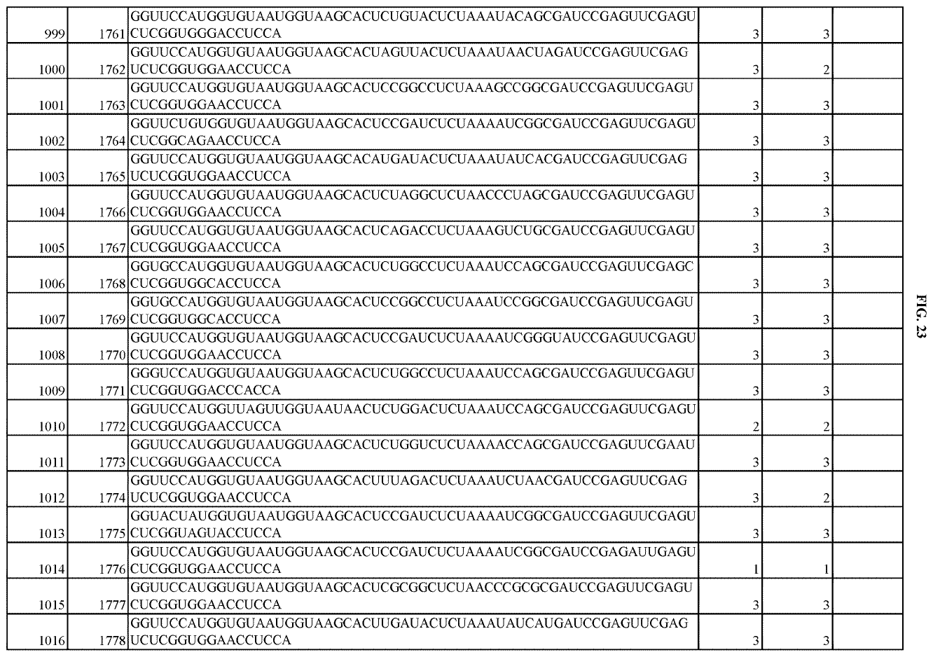

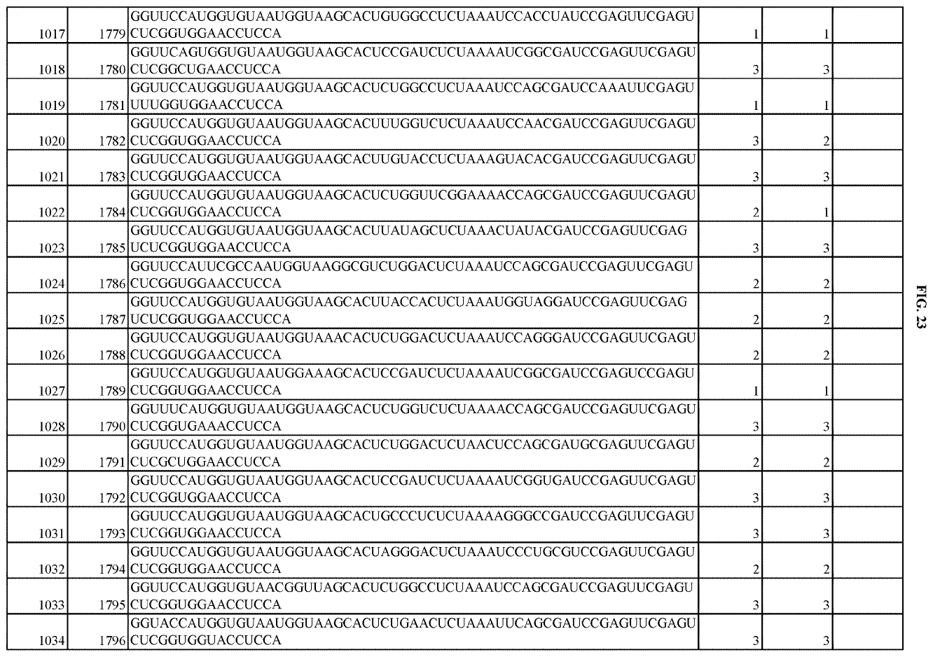

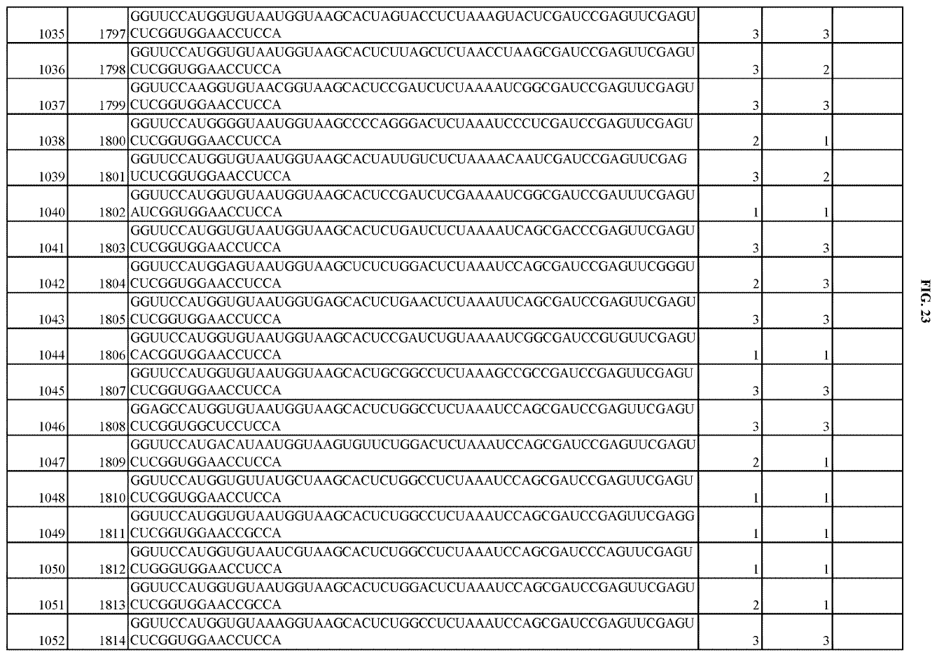

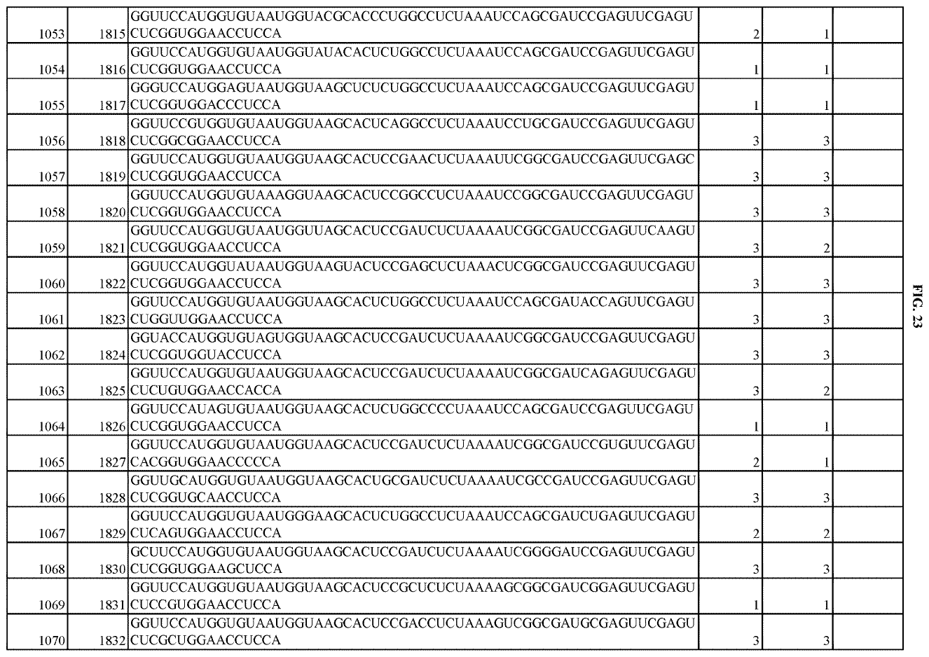

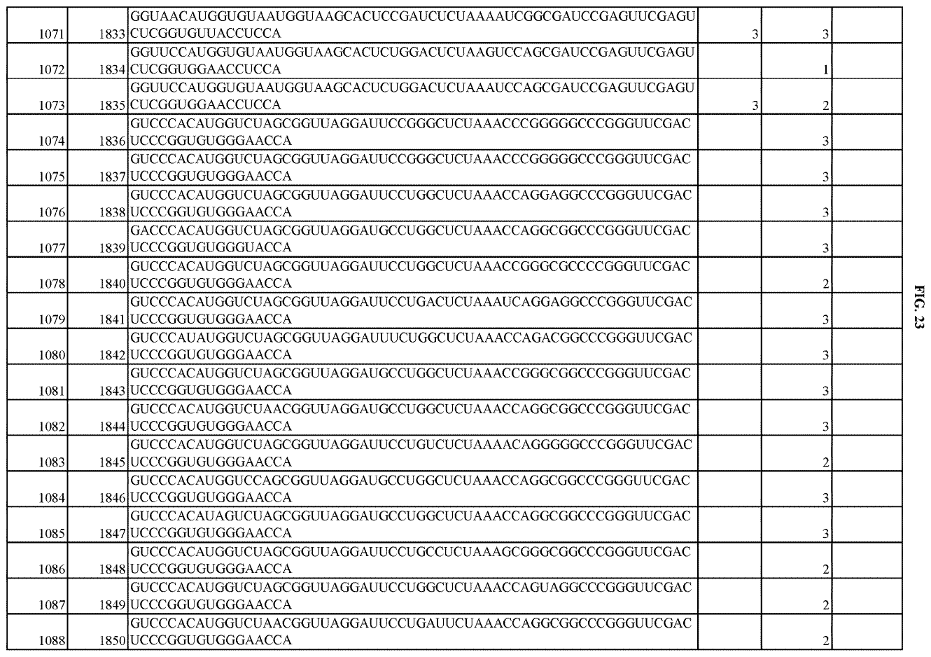

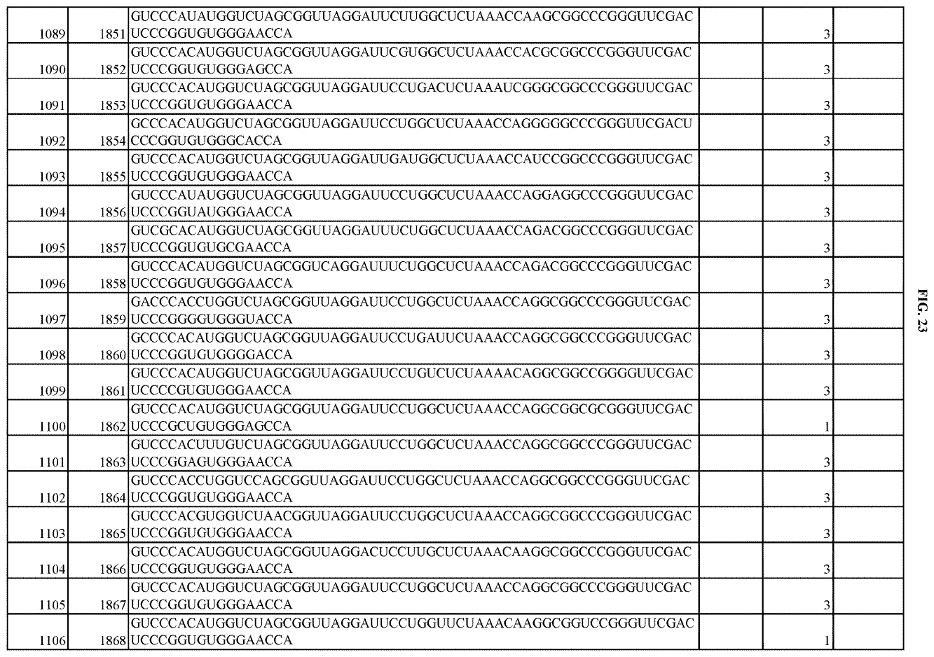

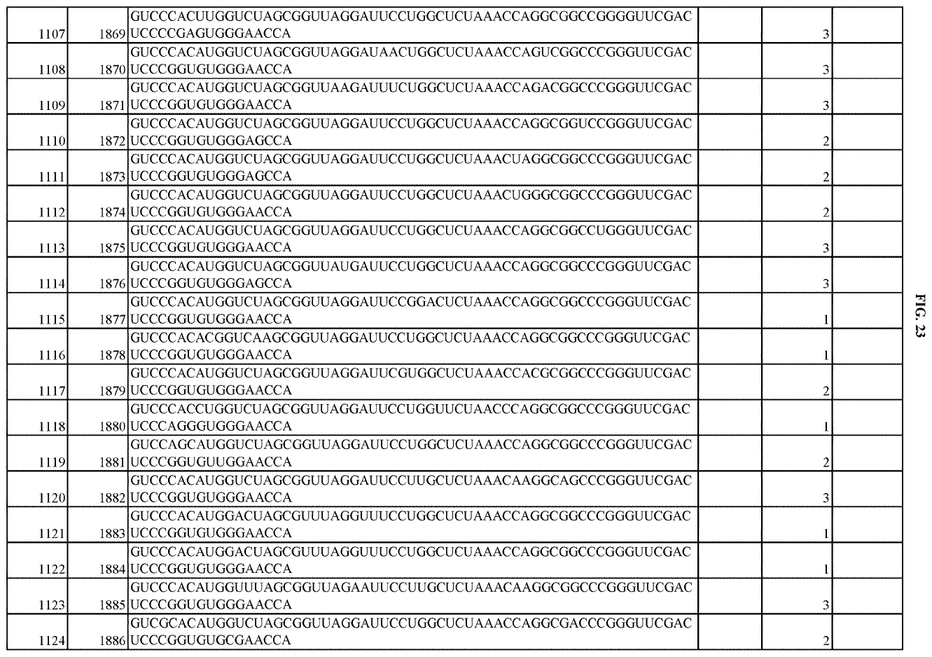

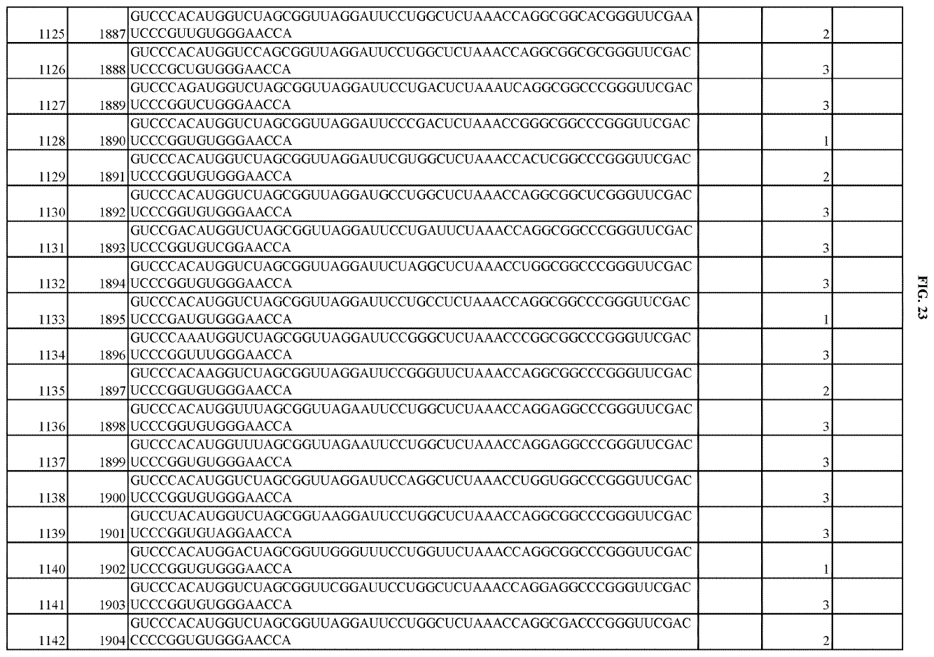

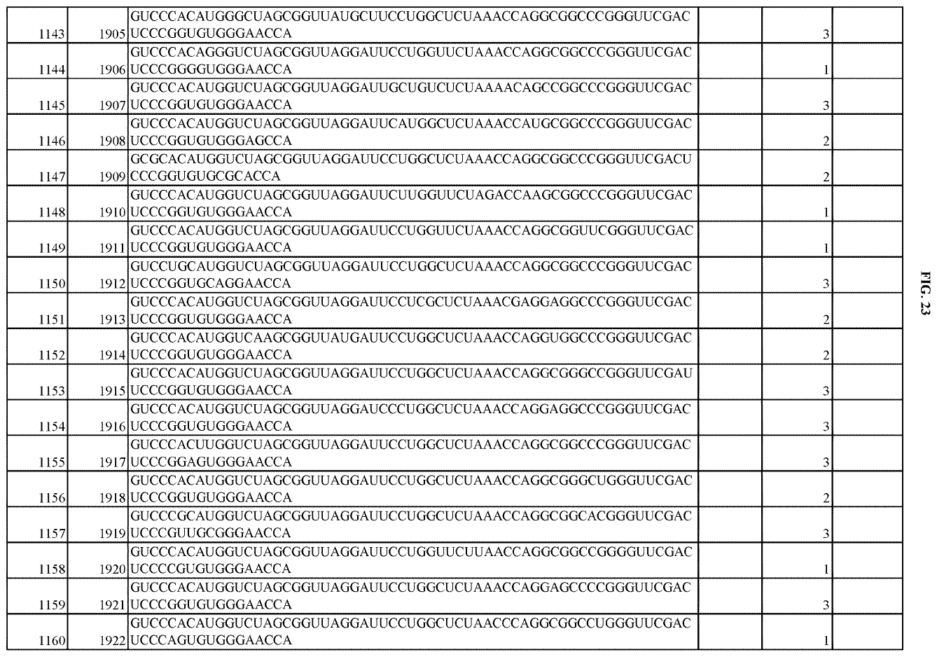

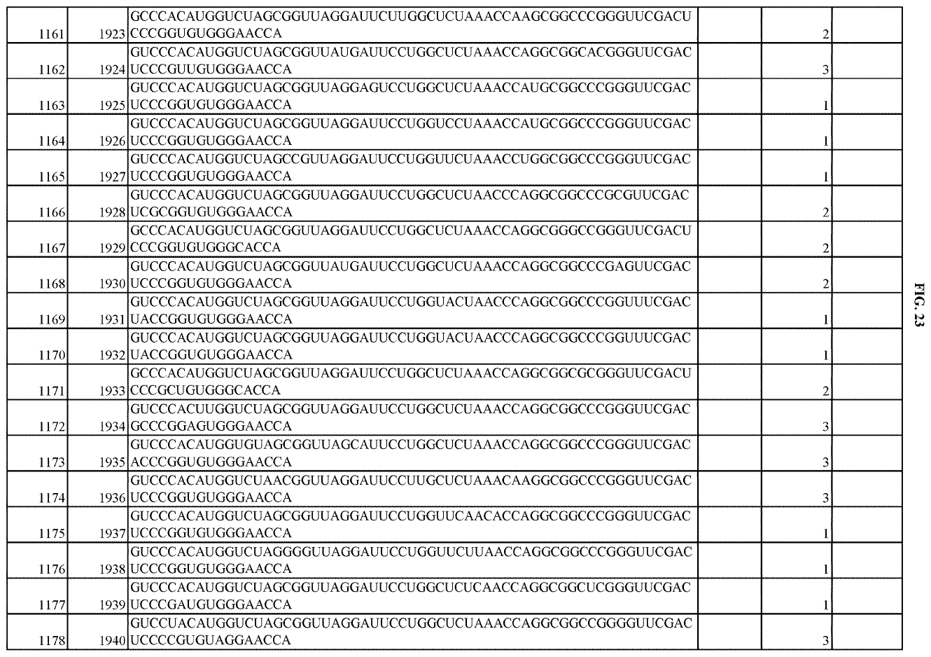

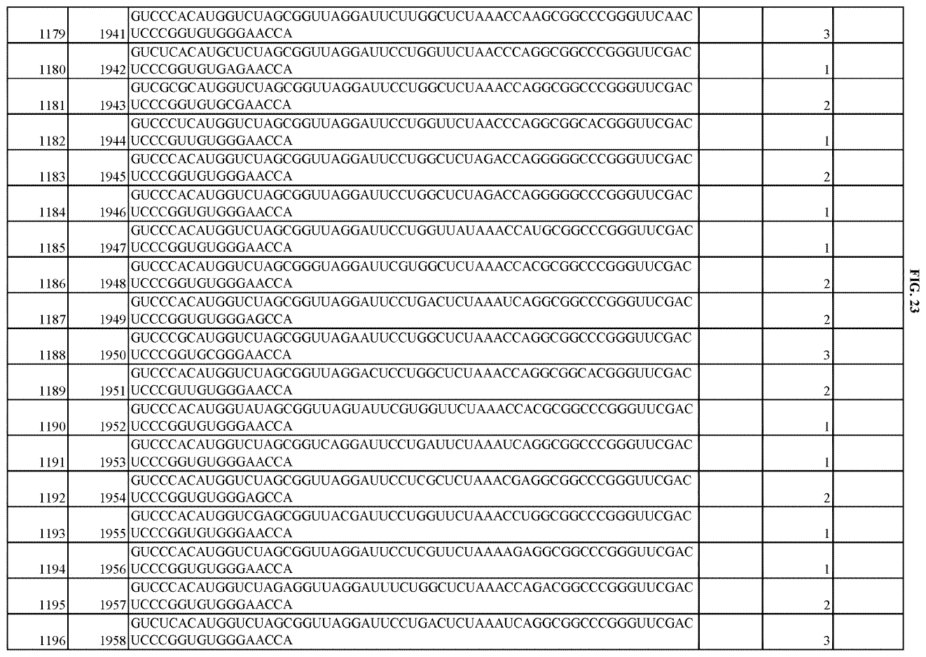

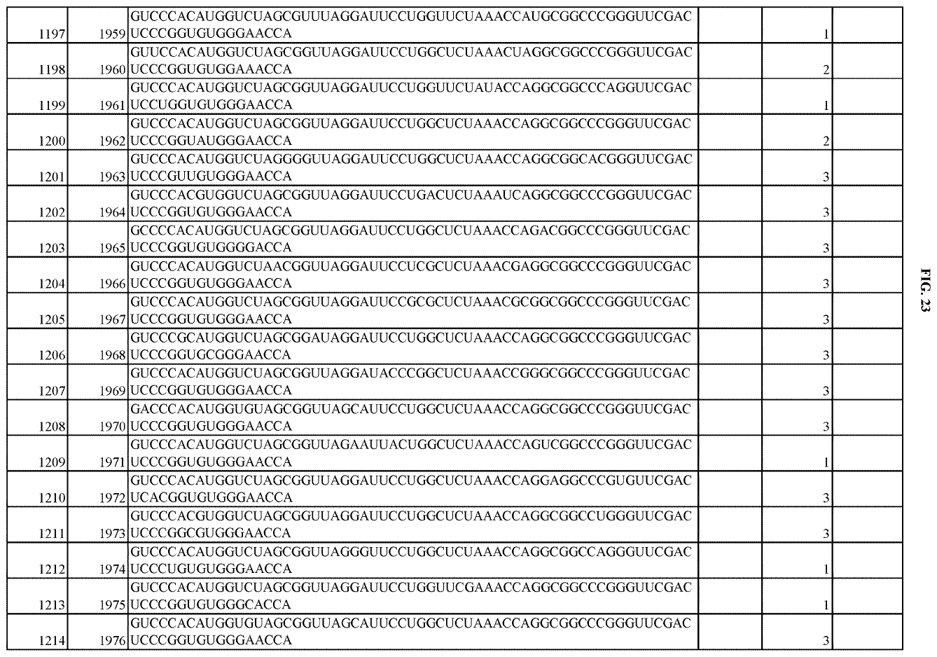

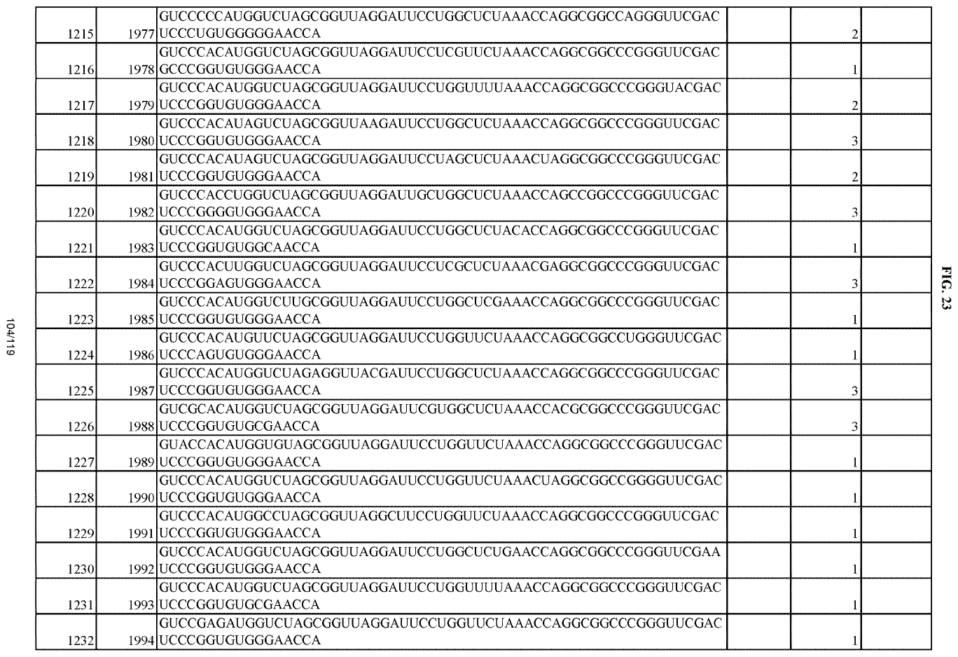

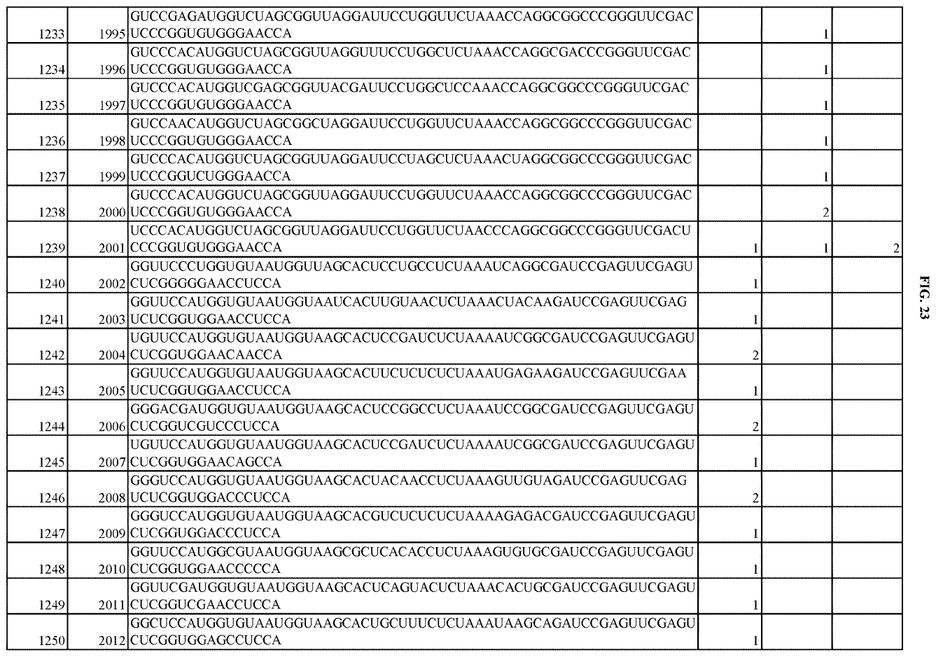

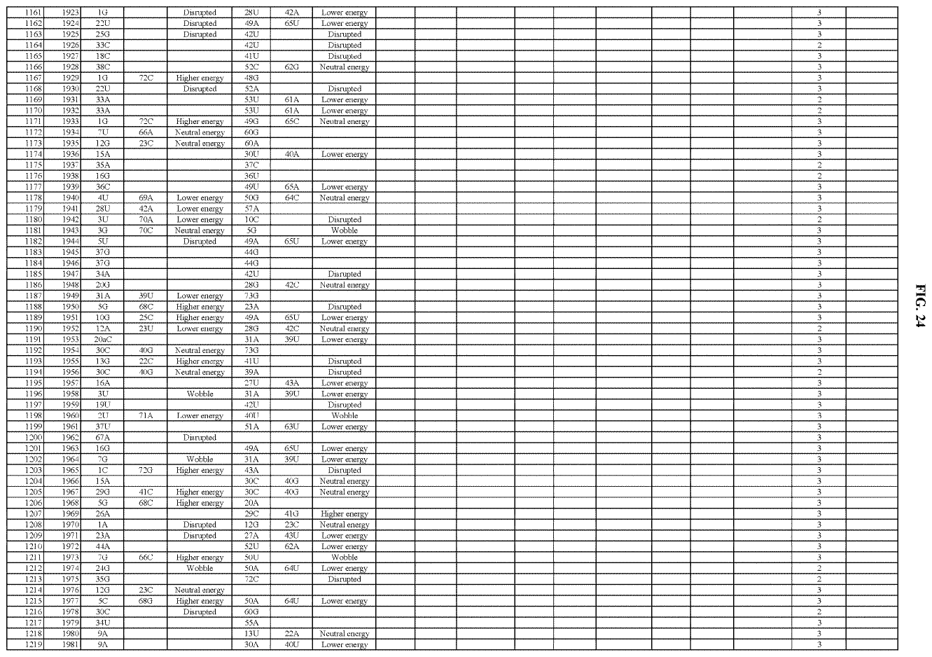

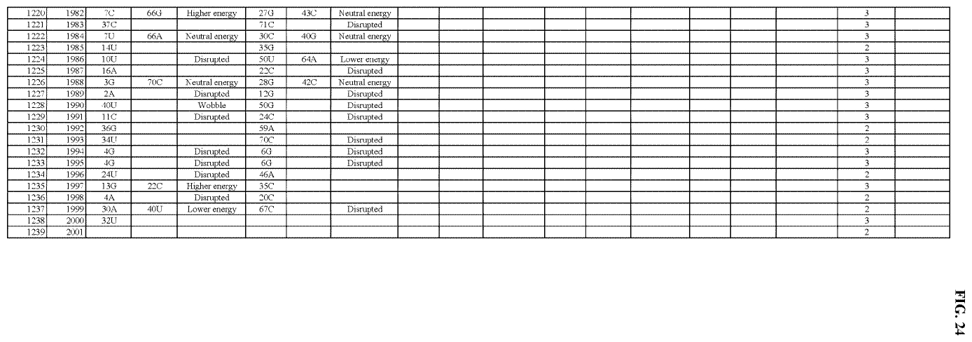

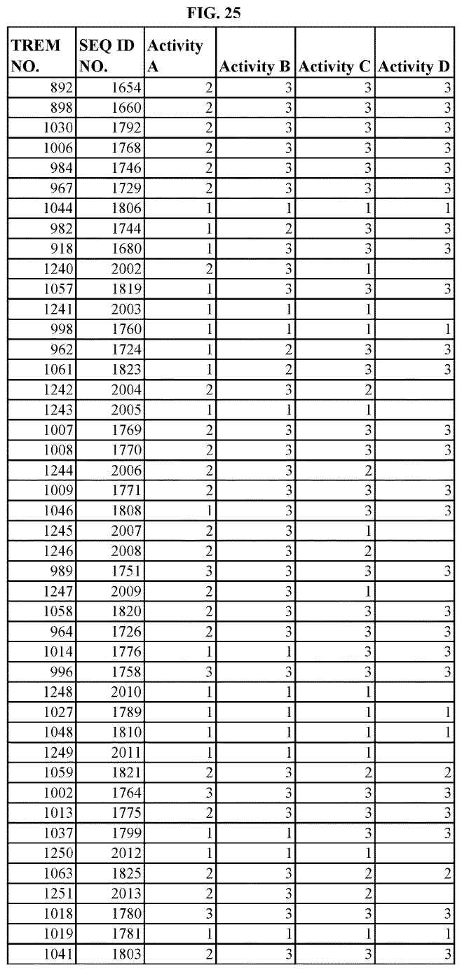

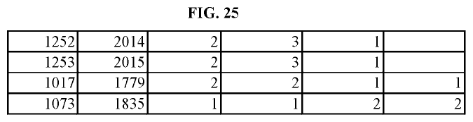

In an embodiment, delivery of a TREM comprising a nucleotide substitution does not affect the activity of the TREM, e.g., the PTC read-through activity of the TREM. In an embodiment, the PTC read-through activity of a TREM delivered as a synthesized oligonucleotide or by lentivirus is about equivalent, e.g., as provided in FIG. 22 and FIG. 25. In an embodiment, the normalized log 2 activity of a TREM comprising nucleotide substitutions delivered by lentivirus is between about −2 and about 4. In an embodiment, the normalized log 2 activity of a TREM comprising nucleotide substitutions delivered as a synthesized oligonucleotide, e.g., oligo, is between about −2.1 and about 4.

In an embodiment, a TREM comprises a nucleotide substitution at a nucleotide position relative to a nucleotide position within reference sequence, e.g., a reference TREM. In an embodiment, the TREM comprising the nucleotide substitution modulates, e.g., increases or decreases, a functional parameter of a TREM, e.g., the activity or stability of the TREM, relative to a reference TREM. In an embodiment, the functional parameter of a TREM that is modulated by nucleotide substitution relative to a reference TREM is the premature termination codon (PTC) read-through activity of the TREM. In an embodiment, the PTC read-through activity of a TREM is modulated by a nucleotide substitution relative to a reference TREM, e.g., a reference TREM of any of SEQ ID NOs: 1638, 1654, 1660, 1835, 1867, 2000, 2001, 2016, 2017, 2018, 2019, 2020, 2021, 2022, or 2023. In an embodiment, the PTC read-through activity of a TREM is modulated by a nucleotide substitution relative to SEQ ID NO: 1638. In an embodiment, the PTC read-through activity of a TREM is modulated by a nucleotide substitution relative to SEQ ID NO: 1654. In an embodiment, the PTC read-through activity of a TREM is modulated by a nucleotide substitution relative to SEQ ID NO: 1660. In an embodiment, the PTC read-through activity of a TREM is modulated by a nucleotide substitution relative to SEQ ID NO: 1835. In an embodiment, the PTC read-through activity of a TREM is modulated by a nucleotide substitution relative to SEQ ID NO: 1867. In an embodiment, the PTC read-through activity of a TREM is modulated by a nucleotide substitution relative to SEQ ID NO: 2000. In an embodiment, the PTC read-through activity of a TREM is modulated by a nucleotide substitution relative to SEQ ID NO: 2001. In an embodiment, the PTC read-through activity of a TREM is modulated by a nucleotide substitution relative to SEQ ID NO: 2016. In an embodiment, the PTC read-through activity of a TREM is modulated by a nucleotide substitution relative to SEQ ID NO: 2017. In an embodiment, the PTC read-through activity of a TREM is modulated by a nucleotide substitution relative to SEQ ID NO: 2018. In an embodiment, the PTC read-through activity of a TREM is modulated by a nucleotide substitution relative to SEQ ID NO: 2019. In an embodiment, the PTC read-through activity of a TREM is modulated by a nucleotide substitution relative to SEQ ID NO: 2020. In an embodiment, the PTC read-through activity of a TREM is modulated by a nucleotide substitution relative to SEQ ID NO: 2021. In an embodiment, the PTC read-through activity of a TREM is modulated by a nucleotide substitution relative to SEQ ID NO: 2022. In an embodiment, the PTC read-through activity of a TREM is modulated by a nucleotide substitution relative to SEQ ID NO: 2023. In an embodiment, the PTC read-through activity of a TREM is increased by a nucleotide substitution relative to a parent TREM, e.g., a parent TREM of any of SEQ ID NOs: 1638, 1654, 1660, 1835, 1867, 2000, 2001, 2016, 2017, 2018, 2019, 2020, 2021, 2022, or 2023. In an embodiment, the PTC read-through activity of a TREM is increased by a nucleotide substitution relative to SEQ ID NO: 1638. In an embodiment, the PTC read-through activity of a TREM is increased by a nucleotide substitution relative to SEQ ID NO: 1654. In an embodiment, the PTC read-through activity of a TREM is increased by a nucleotide substitution relative to SEQ ID NO: 1660. In an embodiment, the PTC read-through activity of a TREM is increased by a nucleotide substitution relative to SEQ ID NO: 1835. In an embodiment, the PTC read-through activity of a TREM is increased by a nucleotide substitution relative to SEQ ID NO: 1867. In an embodiment, the PTC read-through activity of a TREM is increased by a nucleotide substitution relative to SEQ ID NO: 2000. In an embodiment, the PTC read-through activity of a TREM is increased by a nucleotide substitution relative to SEQ ID NO: 2001. In an embodiment, the PTC read-through activity of a TREM is increased by a nucleotide substitution relative to SEQ ID NO: 2016. In an embodiment, the PTC read-through activity of a TREM is increased by a nucleotide substitution relative to SEQ ID NO: 2017. In an embodiment, the PTC read-through activity of a TREM is increased by a nucleotide substitution relative to SEQ ID NO: 2018. In an embodiment, the PTC read-through activity of a TREM is increased by a nucleotide substitution relative to SEQ ID NO: 2019. In an embodiment, the PTC read-through activity of a TREM is increased by a nucleotide substitution relative to SEQ ID NO: 2020. In an embodiment, the PTC read-through activity of a TREM is increased by a nucleotide substitution relative to SEQ ID NO: 2021. In an embodiment, the PTC read-through activity of a TREM is increased by a nucleotide substitution relative to SEQ ID NO: 2022. In an embodiment, the PTC read-through activity of a TREM is increased by a nucleotide substitution relative to SEQ ID NO: 2023.

In an embodiment, a nucleotide substitution relative to a reference TREM, e.g., a reference TREM of any of SEQ ID NOs: 1638, 1654, 1660, 1835, 1867, 2000, 2001, 2016, 2017, 2018, 2019, 2020, 2021, 2022, or 2023, increases the PTC read-through of a TREM by about 1% to about 500% or more, e.g., about 1%, 5%, 10%, 20%, 30%, 40%, 50%, 60%, 70%, 80%, 90%, 100%, 150%, 200%, 250%, 300%, 350%, 400%, 450%, 500%, or more than 500%. In an embodiment, a nucleotide substitution relative to a reference TREM, e.g., a reference TREM of any of SEQ ID NOs: 1638, 1654, 1660, 1835, 1867, 2000, 2001, 2016, 2017, 2018, 2019, 2020, 2021, 2022, or 2023, increases the PTC read-through of a TREM by about 1%. In an embodiment, a nucleotide substitution relative to a reference TREM, e.g., a reference TREM of any of SEQ ID NOs: 1638, 1654, 1660, 1835, 1867, 2000, 2001, 2016, 2017, 2018, 2019, 2020, 2021, 2022, or 2023, increases the PTC read-through of a TREM by about 5%. In an embodiment, a nucleotide substitution relative to a reference TREM, e.g., a parent TREM of any of SEQ ID NOs: 1638, 1654, 1660, 1835, 1867, 2000, 2001, 2016, 2017, 2018, 2019, 2020, 2021, 2022, or 2023, increases the PTC read-through of a TREM by about 10%. In an embodiment, a nucleotide substitution relative to a reference TREM, e.g., a reference TREM of any of SEQ ID NOs: 1638, 1654, 1660, 1835, 1867, 2000, 2001, 2016, 2017, 2018, 2019, 2020, 2021, 2022, or 2023, increases the PTC read-through of a TREM by about 20%. In an embodiment, a nucleotide substitution relative to a reference TREM, e.g., a reference TREM of any of SEQ ID NOs: 1638, 1654, 1660, 1835, 1867, 2000, 2001, 2016, 2017, 2018, 2019, 2020, 2021, 2022, or 2023, increases the PTC read-through of a TREM by about 30%. In an embodiment, a nucleotide substitution relative to a reference TREM, e.g., a reference TREM of any of SEQ ID NOs: 1638, 1654, 1660, 1835, 1867, 2000, 2001, 2016, 2017, 2018, 2019, 2020, 2021, 2022, or 2023, increases the PTC read-through of a TREM by about 40%. In an embodiment, a nucleotide substitution relative to a reference TREM, e.g., a reference TREM of any of SEQ ID NOs: 1638, 1654, 1660, 1835, 1867, 2000, 2001, 2016, 2017, 2018, 2019, 2020, 2021, 2022, or 2023, increases the PTC read-through of a TREM by about 50%. In an embodiment, a nucleotide substitution relative to a reference TREM, e.g., a reference TREM of any of SEQ ID NOs: 1638, 1654, 1660, 1835, 1867, 2000, 2001, 2016, 2017, 2018, 2019, 2020, 2021, 2022, or 2023, increases the PTC read-through of a TREM by about 60%. In an embodiment, a nucleotide substitution relative to a reference TREM, e.g., a reference TREM of any of SEQ ID NOs: 1638, 1654, 1660, 1835, 1867, 2000, 2001, 2016, 2017, 2018, 2019, 2020, 2021, 2022, or 2023, increases the PTC read-through of a TREM by about 70%. In an embodiment, a nucleotide substitution relative to a reference TREM, e.g., a reference TREM of any of SEQ ID NOs: 1638, 1654, 1660, 1835, 1867, 2000, 2001, 2016, 2017, 2018, 2019, 2020, 2021, 2022, or 2023, increases the PTC read-through of a TREM by about 80%. In an embodiment, a nucleotide substitution relative to a reference TREM, e.g., a reference TREM of any of SEQ ID NOs: 1638, 1654, 1660, 1835, 1867, 2000, 2001, 2016, 2017, 2018, 2019, 2020, 2021, 2022, or 2023, increases the PTC read-through of a TREM by about 90%. In an embodiment, a nucleotide substitution relative to a reference TREM, e.g., a reference TREM of any of SEQ ID NOs: 1638, 1654, 1660, 1835, 1867, 2000, 2001, 2016, 2017, 2018, 2019, 2020, 2021, 2022, or 2023, increases the PTC read-through of a TREM by about 100%. In an embodiment, a nucleotide substitution relative to a reference TREM, e.g., a reference TREM of any of SEQ ID NOs: 1638, 1654, 1660, 1835, 1867, 2000, 2001, 2016, 2017, 2018, 2019, 2020, 2021, 2022, or 2023, increases the PTC read-through of a TREM by about 150%. In an embodiment, a nucleotide substitution relative to a reference TREM, e.g., a reference TREM of any of SEQ ID NOs: 1638, 1654, 1660, 1835, 1867, 2000, 2001, 2016, 2017, 2018, 2019, 2020, 2021, 2022, or 2023, increases the PTC read-through of a TREM by about 200%. In an embodiment, a nucleotide substitution relative to a reference TREM, e.g., a reference TREM of any of SEQ ID NOs: 1638, 1654, 1660, 1835, 1867, 2000, 2001, 2016, 2017, 2018, 2019, 2020, 2021, 2022, or 2023, increases the PTC read-through of a TREM by about 250%. In an embodiment, a nucleotide substitution relative to a reference TREM, e.g., a reference TREM of any of SEQ ID NOs: 1638, 1654, 1660, 1835, 1867, 2000, 2001, 2016, 2017, 2018, 2019, 2020, 2021, 2022, or 2023, increases the PTC read-through of a TREM by about 300%. In an embodiment, a nucleotide substitution relative to a reference TREM, e.g., a reference TREM of any of SEQ ID NOs: 1638, 1654, 1660, 1835, 1867, 2000, 2001, 2016, 2017, 2018, 2019, 2020, 2021, 2022, or 2023, increases the PTC read-through of a TREM by about 350%. In an embodiment, a nucleotide substitution relative to a reference TREM, e.g., a reference TREM of any of SEQ ID NOs: 1638, 1654, 1660, 1835, 1867, 2000, 2001, 2016, 2017, 2018, 2019, 2020, 2021, 2022, or 2023, increases the PTC read-through of a TREM by about 400%. In an embodiment, a nucleotide substitution relative to a reference TREM, e.g., a reference TREM of any of SEQ ID NOs: 1638, 1654, 1660, 1835, 1867, 2000, 2001, 2016, 2017, 2018, 2019, 2020, 2021, 2022, or 2023, increases the PTC read-through of a TREM by about 450%. In an embodiment, a nucleotide substitution relative to a reference TREM, e.g., a reference TREM of any of SEQ ID NOs: 1638, 1654, 1660, 1835, 1867, 2000, 2001, 2016, 2017, 2018, 2019, 2020, 2021, 2022, or 2023, increases the PTC read-through of a TREM by about 500%. In an embodiment, a nucleotide substitution relative to a reference TREM, e.g., a reference TREM of any of SEQ ID NOs: 1638, 1654, 1660, 1835, 1867, 2000, 2001, 2016, 2017, 2018, 2019, 2020, 2021, 2022, or 2023, increases the PTC read-through of a TREM by more than about 500%.

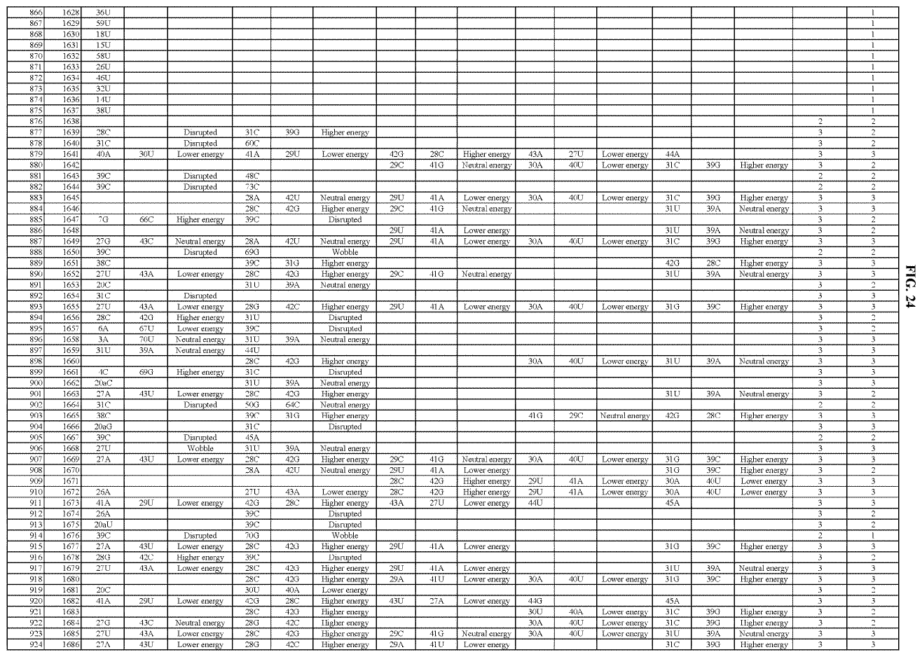

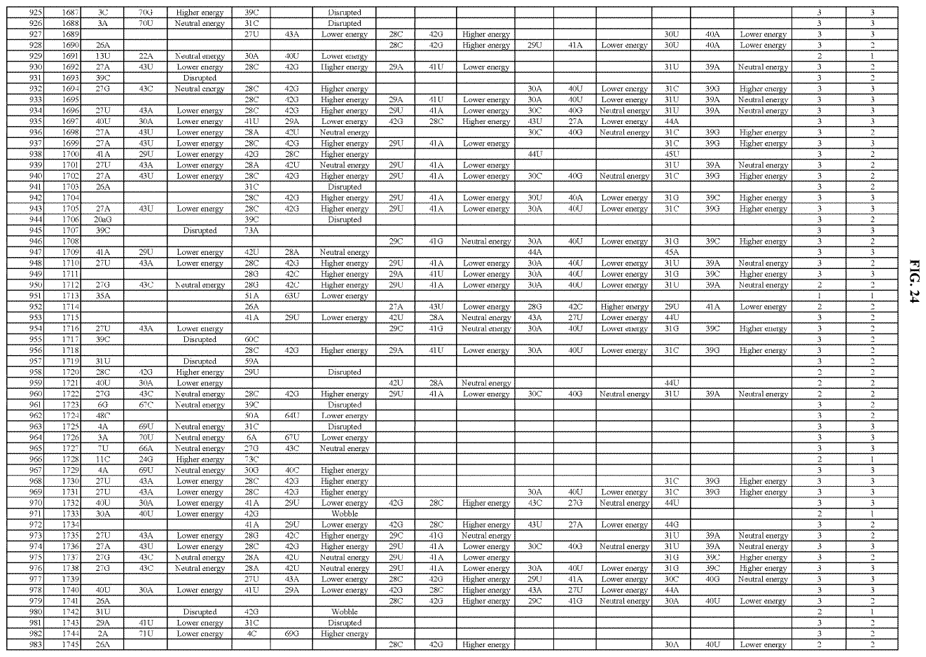

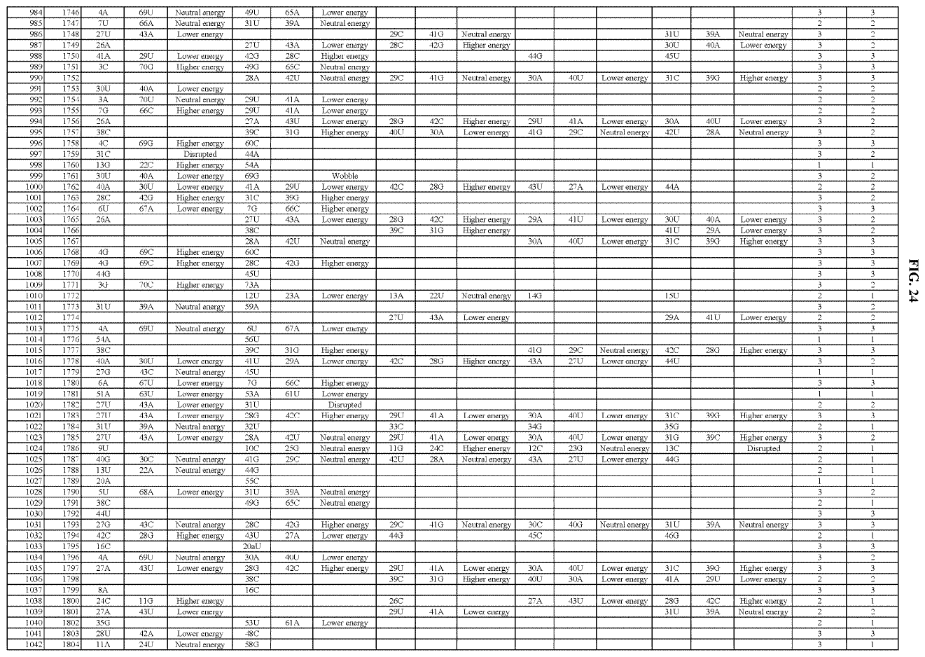

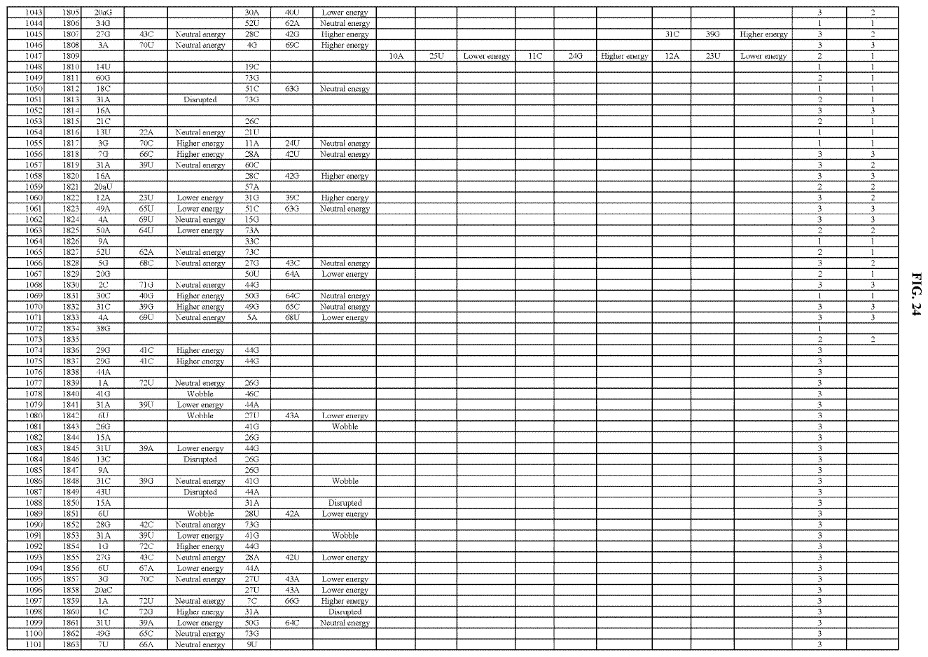

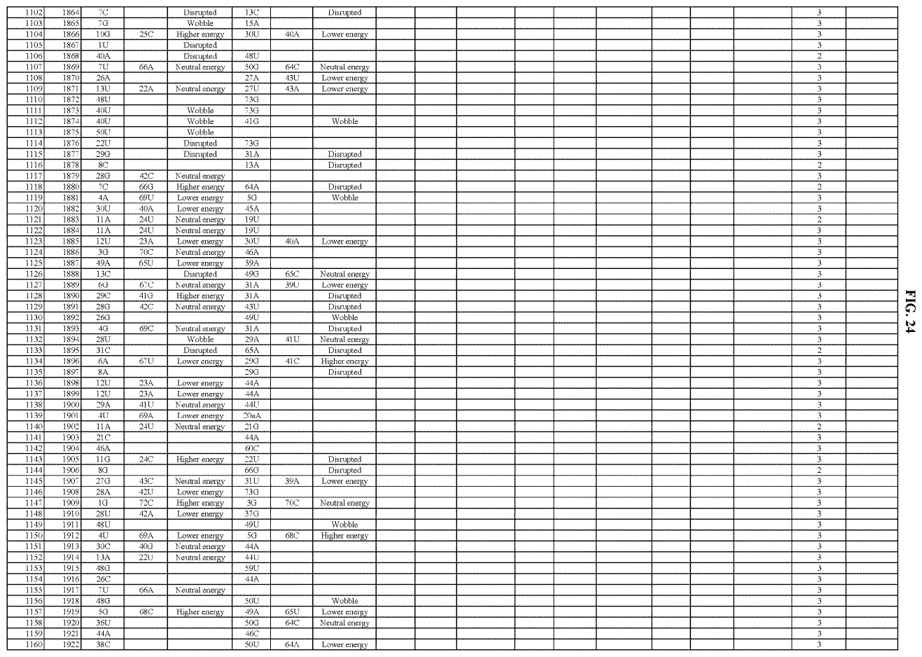

In an embodiment, a nucleotide substitution relative to a reference TREM, e.g., a reference TREM of any of SEQ ID NOs: 1638, 1654, 1660, 1835, 1867, 2000, 2001, 2016, 2017, 2018, 2019, 2020, 2021, 2022, or 2023, increases the PTC read-through of a TREM by about 2-fold to about 500-fold or more, e.g., about 2-fold, 5-fold, 10-fold, 20-fold, 30-fold, 40-fold, 50-fold, 60-fold, 70-fold, 80-fold, 90-fold, 100-fold, 150-fold, 200-fold, 250-fold, 300-fold, 350-fold, 400-fold, 450-fold, 500-fold, or more than 500-fold. In an embodiment, a nucleotide substitution relative to a reference TREM, e.g., a reference TREM of any of SEQ ID NOs: 1638, 1654, 1660, 1835, 1867, 2000, 2001, 2016, 2017, 2018, 2019, 2020, 2021, 2022, or 2023, increases the PTC read-through of a TREM by about 2-fold. In an embodiment, a nucleotide substitution relative to a reference TREM, e.g., a reference TREM of any of SEQ ID NOs: 1638, 1654, 1660, 1835, 1867, 2000, 2001, 2016, 2017, 2018, 2019, 2020, 2021, 2022, or 2023, increases the PTC read-through of a TREM by about 5-fold. In an embodiment, a nucleotide substitution relative to a reference TREM, e.g., a reference TREM of any of SEQ ID NOs: 1638, 1654, 1660, 1835, 1867, 2000, 2001, 2016, 2017, 2018, 2019, 2020, 2021, 2022, or 2023, increases the PTC read-through of a TREM by about 10-fold. In an embodiment, a nucleotide substitution relative to a reference TREM, e.g., a reference TREM of any of SEQ ID NOs: 1638, 1654, 1660, 1835, 1867, 2000, 2001, 2016, 2017, 2018, 2019, 2020, 2021, 2022, or 2023, increases the PTC read-through of a TREM by about 20-fold. In an embodiment, a nucleotide substitution relative to a reference TREM, e.g., a reference TREM of any of SEQ ID NOs: 1638, 1654, 1660, 1835, 1867, 2000, 2001, 2016, 2017, 2018, 2019, 2020, 2021, 2022, or 2023, increases the PTC read-through of a TREM by about 30-fold. In an embodiment, a nucleotide substitution relative to a reference TREM, e.g., a reference TREM of any of SEQ ID NOs: 1638, 1654, 1660, 1835, 1867, 2000, 2001, 2016, 2017, 2018, 2019, 2020, 2021, 2022, or 2023, increases the PTC read-through of a TREM by about 40-fold. In an embodiment, a nucleotide substitution relative to a reference TREM, e.g., a reference TREM of any of SEQ ID NOs: 1638, 1654, 1660, 1835, 1867, 2000, 2001, 2016, 2017, 2018, 2019, 2020, 2021, 2022, or 2023, increases the PTC read-through of a TREM by about 50-fold. In an embodiment, a nucleotide substitution relative to a reference TREM, e.g., a reference TREM of any of SEQ ID NOs: 1638, 1654, 1660, 1835, 1867, 2000, 2001, 2016, 2017, 2018, 2019, 2020, 2021, 2022, or 2023, increases the PTC read-through of a TREM by about 60-fold. In an embodiment, a nucleotide substitution relative to a reference TREM, e.g., a reference TREM of any of SEQ ID NOs: 1638, 1654, 1660, 1835, 1867, 2000, 2001, 2016, 2017, 2018, 2019, 2020, 2021, 2022, or 2023, increases the PTC read-through of a TREM by about 70-fold. In an embodiment, a nucleotide substitution relative to a reference TREM, e.g., a reference TREM of any of SEQ ID NOs: 1638, 1654, 1660, 1835, 1867, 2000, 2001, 2016, 2017, 2018, 2019, 2020, 2021, 2022, or 2023, increases the PTC read-through of a TREM by about 80-fold. In an embodiment, a nucleotide substitution relative to a reference TREM, e.g., a reference TREM of any of SEQ ID NOs: 1638, 1654, 1660, 1835, 1867, 2000, 2001, 2016, 2017, 2018, 2019, 2020, 2021, 2022, or 2023, increases the PTC read-through of a TREM by about 90-fold. In an embodiment, a nucleotide substitution relative to a reference TREM, e.g., a reference TREM of any of SEQ ID NOs: 1638, 1654, 1660, 1835, 1867, 2000, 2001, 2016, 2017, 2018, 2019, 2020, 2021, 2022, or 2023, increases the PTC read-through of a TREM by about 100-fold. In an embodiment, a nucleotide substitution relative to a reference TREM, e.g., a reference TREM of any of SEQ ID NOs: 1638, 1654, 1660, 1835, 1867, 2000, 2001, 2016, 2017, 2018, 2019, 2020, 2021, 2022, or 2023, increases the PTC read-through of a TREM by about 150-fold. In an embodiment, a nucleotide substitution relative to a reference TREM, e.g., a reference TREM of any of SEQ ID NOs: 1638, 1654, 1660, 1835, 1867, 2000, 2001, 2016, 2017, 2018, 2019, 2020, 2021, 2022, or 2023, increases the PTC read-through of a TREM by about 200-fold. In an embodiment, a nucleotide substitution relative to a reference TREM, e.g., a reference TREM of any of SEQ ID NOs: 1638, 1654, 1660, 1835, 1867, 2000, 2001, 2016, 2017, 2018, 2019, 2020, 2021, 2022, or 2023, increases the PTC read-through of a TREM by about 250-fold. In an embodiment, a nucleotide substitution relative to a reference TREM, e.g., a reference TREM of any of SEQ ID NOs: 1638, 1654, 1660, 1835, 1867, 2000, 2001, 2016, 2017, 2018, 2019, 2020, 2021, 2022, or 2023, increases the PTC read-through of a TREM by about 300-fold. In an embodiment, a nucleotide substitution relative to a reference TREM, e.g., a reference TREM of any of SEQ ID NOs: 1638, 1654, 1660, 1835, 1867, 2000, 2001, 2016, 2017, 2018, 2019, 2020, 2021, 2022, or 2023, increases the PTC read-through of a TREM by about 350-fold. In an embodiment, a nucleotide substitution relative to a reference TREM, e.g., a reference TREM of any of SEQ ID NOs: 1638, 1654, 1660, 1835, 1867, 2000, 2001, 2016, 2017, 2018, 2019, 2020, 2021, 2022, or 2023, increases the PTC read-through of a TREM by about 400-fold. In an embodiment, a nucleotide substitution relative to a reference TREM, e.g., a reference TREM of any of SEQ ID NOs: 1638, 1654, 1660, 1835, 1867, 2000, 2001, 2016, 2017, 2018, 2019, 2020, 2021, 2022, or 2023, increases the PTC read-through of a TREM by about 450-fold. In an embodiment, a nucleotide substitution relative to a reference TREM, e.g., a reference TREM of any of SEQ ID NOs: 1638, 1654, 1660, 1835, 1867, 2000, 2001, 2016, 2017, 2018, 2019, 2020, 2021, 2022, or 2023, increases the PTC read-through of a TREM by about 500-fold. In an embodiment, a nucleotide substitution relative to a reference TREM, e.g., a reference TREM of any of SEQ ID NOs: 1638, 1654, 1660, 1835, 1867, 2000, 2001, 2016, 2017, 2018, 2019, 2020, 2021, 2022, or 2023, increases the PTC read-through of a TREM by more than about 500-fold. In an embodiment, a nucleotide substitution, e.g., a nucleotide substitution listed in FIG. 24, modulates activity of a TREM relative to a reference TREM. In an embodiment, a nucleotide substitution listed in FIG. 24 increases the activity of a TREM relative to a reference TREM. In an embodiment, a nucleotide substitution listed in FIG. 24 decreases the activity of a TREM relative to a reference TREM.

In an embodiment, a TREM having a nucleotide substitution is a TREM listed in FIG. 24, e.g., any of SEQ ID NOs: 1457-2001. In an embodiment, the nucleotide substitution is a nucleotide substitution listed in FIG. 24. In an embodiment, the nucleotide substitution increases premature termination codon (PTC) read-through activity of the TREM, e.g., as provided in FIG. 24. In an embodiment, the nucleotide substitution that increases PTC read-through activity of a TREM is a nucleotide substitution comprised by any of SEQ ID NOs: 1469, 1473, 1480, 1639, 1640, 1641, 1642, 1645, 1646, 1649, 1651, 1652, 1654, 1655, 1658, 1659, 1660, 1661, 1662, 1665, 1666, 1668, 1669, 1671, 1672, 1673, 1677, 1679, 1680, 1682, 1685, 1686, 1687, 1688, 1689, 1694, 1695, 1696, 1697, 1699, 1704, 1705, 1707, 1709, 1711, 1725, 1726, 1727, 1729, 1730, 1731, 1732, 1736, 1738, 1739, 1740, 1746, 1750, 1751, 1752, 1758, 1764, 1767, 1768, 1769, 1770, 1775, 1777, 1780, 1783, 1792, 1793, 1795, 1797, 1799, 1803, 1808, 1814, 1818, 1820, 1823, 1824, 1830, 1832, 1833.

In an embodiment, a TREM comprising an Arg-TGA scaffold, e.g., a TREM corresponding to a reference sequence, e.g., SEQ ID NO: 1638, comprises a nucleotide substitution at any one of the positions shown in FIG. 6. In an embodiment, a TREM comprising an Arg-TGA scaffold, e.g., a TREM corresponding to a reference sequence, e.g., SEQ ID NO: 1638, comprises a nucleotide substitution at any of nucleotide positions 1, 2, 3, 4, 5, 6, or 7, e.g., in the [ASt Domain1], e.g., as shown in FIG. 6. In an embodiment, a TREM comprising an Arg-TGA scaffold, e.g., a TREM corresponding to a reference sequence, e.g., SEQ ID NO: 1638, comprises a nucleotide substitution at any of nucleotide positions 32, 33, 34, 35, 36, 37, or 38, e.g., in the [ACH Domain], e.g., as shown in FIG. 6. In an embodiment, a TREM comprising an Arg-TGA scaffold, e.g., a TREM corresponding to a reference sequence, e.g., SEQ ID NO: 1638, comprises a nucleotide substitution at any of nucleotide positions 65, 66, 67, 68, 69, 70, 71, 72, or 73, e.g., in the [ASt Domain2], e.g., as shown in FIG. 6. In an embodiment, the activity of a TREM comprising an Arg-TGA scaffold, e.g., a TREM corresponding to a reference sequence, e.g., SEQ ID NO: 1638, comprising a nucleotide substitution at any of nucleotide positions 1, 2, 3, 4, 5, 6, or 7 is increased, e.g., relative to a reference sequence, e.g., increased by about 1.5-fold, about 2-fold, about 3-fold, or about 3.6-fold, e.g., as shown in FIG. 6. In an embodiment, the activity of a TREM comprising an Arg-TGA scaffold, e.g., a TREM corresponding to a reference sequence, e.g., SEQ ID NO: 1638, comprising a nucleotide substitution at any of nucleotide positions 32, 33, 34, 35, 36, 37, or 38 is decreased, e.g., relative to a reference sequence, e.g., decreased by about 2-fold, e.g., as shown in FIG. 6. In an embodiment, the activity of a TREM comprising an Arg-TGA scaffold, e.g., a TREM corresponding to a reference sequence, e.g., SEQ ID NO: 1638, comprising a nucleotide substitution at any of nucleotide positions 65, 66, 67, 68, 69, 70, 71, 72, or 73 is increased, e.g., relative to a reference sequence, e.g., increased by about 1.5-fold, about 2-fold, or about 3.6-fold, e.g., as shown in FIG. 6.

In an embodiment, a TREM comprising a Gln-TAG scaffold, e.g., a TREM corresponding to a reference sequence, e.g., SEQ ID NO: 1835, comprises a nucleotide substitution at any one of the positions shown in FIG. 7. In an embodiment, a TREM comprising a Gln-TAG scaffold, e.g., a TREM corresponding to a reference sequence, e.g., SEQ ID NO: 1835, comprises a nucleotide substitution at any of nucleotide positions 26, 27, 28, 29, 30, or 31, e.g., in the [ACH Domain], e.g., as shown in FIG. 7. In an embodiment, a TREM comprising a Gln-TAG scaffold, e.g., a TREM corresponding to a reference sequence, e.g., SEQ ID NO: 1835, comprises a nucleotide substitution at any of nucleotide positions 32, 33, 34, 35, 36, 37, or 38, e.g., in the [ACH Domain], e.g., as shown in FIG. 7. In an embodiment, a TREM comprising a Gln-TAG scaffold, e.g., a TREM corresponding to a reference sequence, e.g., SEQ ID NO: 1835, comprises a nucleotide substitution at any of nucleotide positions 39, 40, 41, 42, 43, or 44, e.g., in the [ACH Domain], e.g., as shown in FIG. 7. In an embodiment, the activity of a TREM comprising a Gln-TAG scaffold, e.g., a TREM corresponding to a reference sequence, e.g., SEQ ID NO: 1835, comprising a nucleotide substitution at any of nucleotide positions 26, 27, 28, 29, 30, or 31 is increased, e.g., relative to a reference sequence, e.g., increased by about 1.5-fold, about 5-fold, about 6-fold, about 7-fold, or about 8-fold, e.g., as shown in FIG. 7. In an embodiment, the activity of a TREM comprising a Gln-TAG scaffold, e.g., a TREM corresponding to a reference sequence, e.g., SEQ ID NO: 1835, comprising a nucleotide substitution at any of nucleotide positions 32, 33, 34, 35, 36, 37, or 38 is decreased, e.g., relative to a reference sequence, e.g., decreased by about 2-fold, e.g., as shown in FIG. 7. In an embodiment, the activity of a TREM comprising a Gln-TAG scaffold, e.g., a TREM corresponding to a reference sequence, e.g., SEQ ID NO: 1835, comprising a nucleotide substitution at any of nucleotide positions 39, 40, 41, 42, 43, or 44 is increased, e.g., relative to a reference sequence, e.g., increased by about 2-fold, about 5-fold, about 6-fold, about 7-fold, or about 8-fold, e.g., as shown in FIG. 7.

In an embodiment, a TREM comprising a Gln-TAG scaffold, e.g., a TREM corresponding to a reference sequence, e.g., SEQ ID NO: 1660 or 1654, comprises a nucleotide substitution at any one of the positions shown in FIG. 8. In an embodiment, a TREM comprising a Gln-TAG scaffold, e.g., a TREM corresponding to a reference sequence, e.g., SEQ ID NO: 1660 or 1654, comprises a nucleotide substitution at any of nucleotide positions 3, 4, 5, 6, or 7, e.g., in the [ASt Domain1], e.g., as shown in FIG. 8. In an embodiment, a TREM comprising a Gln-TAG scaffold, e.g., a TREM corresponding to a reference sequence, e.g., SEQ ID NO: 1660 or 1654, comprises a nucleotide substitution at nucleotide position 20a, e.g., in the [DH Domain], e.g., as shown in FIG. 8. In an embodiment, a TREM comprising a Gln-TAG scaffold, e.g., a TREM corresponding to a reference sequence, e.g., SEQ ID NO: 1660 or 1654, comprises a nucleotide substitution at any of nucleotide positions 26, 27, 28, 29, or 30, e.g., in the [ACH Domain], e.g., as shown in FIG. 8. In an embodiment, a TREM comprising a Gln-TAG scaffold, e.g., a TREM corresponding to a reference sequence, e.g., SEQ ID NO: 1660 or 1654, comprises a nucleotide substitution at any of nucleotide positions 32, 33, 34, 35, 36, 37, or 38, e.g., in the [ACH Domain], e.g., as shown in FIG. 8. In an embodiment, a TREM comprising a Gln-TAG scaffold, e.g., a TREM corresponding to a reference sequence, e.g., SEQ ID NO: 1660 or 1654, comprises a nucleotide substitution at any of nucleotide positions 40, 41, 42, 43, 44, or 45, e.g., in the [ACH Domain], e.g., as shown in FIG. 8. In an embodiment, a TREM comprising a Gln-TAG scaffold, e.g., a TREM corresponding to a reference sequence, e.g., SEQ ID NO: 1660 or 1654, comprises a nucleotide substitution at nucleotide position 49, e.g., in the [TH Domain], e.g., as shown in FIG. 8. In an embodiment, a TREM comprising a Gln-TAG scaffold, e.g., a TREM corresponding to a reference sequence, e.g., SEQ ID NO: 1660 or 1654, comprises a nucleotide substitution at any of nucleotide positions 65, 66, 67, 68, 69, 70, 71, 72, or 73, e.g., in the [ASt Domain2], e.g., as shown in FIG. 8. In an embodiment, the activity of a TREM comprising a Gln-TAG scaffold, e.g., a TREM corresponding to a reference sequence, e.g., SEQ ID NO: 1660 or 1654, comprising a nucleotide substitution at any of nucleotide positions 3, 4, 5, 6, or 7 is increased, e.g., relative to a reference sequence, e.g., increased by about 2.5-fold, about 3-fold, or about 3.5-fold, e.g., as shown in FIG. 8. In an embodiment, the activity of a TREM comprising a Gln-TAG scaffold, e.g., a TREM corresponding to a reference sequence, e.g., SEQ ID NO: 1660 or 1654, comprising a nucleotide substitution at nucleotide position 20a is increased, e.g., relative to a reference sequence, e.g., increased by about 2.5-fold or about 3-fold, e.g., as shown in FIG. 8. In an embodiment, the activity of a TREM comprising a Gln-TAG scaffold, e.g., a TREM corresponding to a reference sequence, e.g., SEQ ID NO: 1660 or 1654, comprises a nucleotide substitution at any of nucleotide positions 26, 27, 28, 29, or 30 is increased, e.g., relative to a reference sequence, e.g., increased by about 1.5-fold, about 2-fold, or about 2.5-fold, e.g., as shown in FIG. 8. In an embodiment, the activity of a TREM comprising a Gln-TAG scaffold, e.g., a TREM corresponding to a reference sequence, e.g., SEQ ID NO: 1660 or 1654, comprising a nucleotide substitution at any of nucleotide positions 32, 33, 34, 35, 36, 37, or 38 is decreased, e.g., relative to a reference sequence, e.g., decreased by about 2-fold, e.g., as shown in FIG. 8. In an embodiment, the activity of a TREM comprising a Gln-TAG scaffold, e.g., a TREM corresponding to a reference sequence, e.g., SEQ ID NO: 1660 or 1654, comprising a nucleotide substitution at any of nucleotide positions 40, 41, 42, 43, 44, or 45 is increased, e.g., relative to a reference sequence, e.g., increased by about 1.5-fold, about 2-fold, or about 2.5-fold, e.g., as shown in FIG. 8. In an embodiment, the activity of a TREM comprising a Gln-TAG scaffold, e.g., a TREM corresponding to a reference sequence, e.g., SEQ ID NO: 1660 or 1654, comprising a nucleotide substitution at nucleotide position 49 is increased, e.g., relative to a reference sequence, e.g., increased by about 2-fold, e.g., as shown in FIG. 8. In an embodiment, the activity of a TREM comprising a Gln-TAG scaffold, e.g., a TREM corresponding to a reference sequence, e.g., SEQ ID NO: 1660 or 1654, comprising a nucleotide substitution at any of nucleotide positions 65, 66, 67, 68, 69, 70, 71, 72, or 73 is increased, e.g., relative to a reference sequence, e.g., increased by about 1.5-fold, about 2-fold, about 2.5-fold, about 3-fold, or about 3.5-fold, e.g., as shown in FIG. 8.

In an embodiment, a TREM comprising a Glu-TAG scaffold, e.g., a TREM corresponding to a reference sequence, e.g., SEQ ID NO: 1867, 2000, or 2001, comprises a nucleotide substitution at any one of the positions shown in FIG. 9. In an embodiment, a TREM comprising a Glu-TAG scaffold, e.g., a TREM corresponding to a reference sequence, e.g., SEQ ID NO: 1867, 2000, or 2001, comprises a nucleotide substitution at any of nucleotide positions 1, 2, 3, 4, 5, 6, 7, 8, 9, 10, 11, 12, 13, 14, or 15, e.g., in the [ASt Domain1] or the [DH Domain], e.g., as shown in FIG. 9. In an embodiment, a TREM comprising a Glu-TAG scaffold, e.g., a TREM corresponding to a reference sequence, e.g., SEQ ID NO: 1867, 2000, or 2001, comprises a nucleotide substitution at any of nucleotide positions 20a, 21, 22, 23, 24, 25, 26, 27, 28, 29, 30, or 31, e.g., in the [DH Domain] or the [ACH Domain], e.g., as shown in FIG. 9. In an embodiment, a TREM comprising a Glu-TAG scaffold, e.g., a TREM corresponding to a reference sequence, e.g., SEQ ID NO: 1867, 2000, or 2001, comprises a nucleotide substitution at any of nucleotide positions 32, 33, 34, 35, 36, 37, or 38, e.g., in the [ACH Domain], e.g., as shown in FIG. 9. In an embodiment, a TREM comprising a Glu-TAG scaffold, e.g., a TREM corresponding to a reference sequence, e.g., SEQ ID NO: 1867, 2000, or 2001, comprises a nucleotide substitution at any of nucleotide positions 40, 41, 42, 43, 44, or 45, e.g., in the [ACH Domain], e.g., as shown in FIG. 9. In an embodiment, a TREM comprising a Glu-TAG scaffold, e.g., a TREM corresponding to a reference sequence, e.g., SEQ ID NO: 1867, 2000, or 2001, comprises a nucleotide substitution at any of nucleotide positions 46 or 48, e.g., in the [VL Domain], e.g., as shown in FIG. 9. In an embodiment, a TREM comprising a Glu-TAG scaffold, e.g., a TREM corresponding to a reference sequence, e.g., SEQ ID NO: 1867, 2000, or 2001, comprises a nucleotide substitution at any of nucleotide positions 59 or 60, e.g., in the [TH Domain], e.g., as shown in FIG. 9. In an embodiment, a TREM comprising a Glu-TAG scaffold, e.g., a TREM corresponding to a reference sequence, e.g., SEQ ID NO: 1867, 2000, or 2001, comprises a nucleotide substitution at any of nucleotide positions 66, 67, 68, 69, 70, 71, 72, or 73, e.g., in the [ASt Domain2], e.g., as shown in FIG. 9. In an embodiment, the activity of a TREM comprising a Glu-TAG scaffold, e.g., a TREM corresponding to a reference sequence, e.g., SEQ ID NO: 1867, 2000, or 2001, comprising a nucleotide substitution at any of nucleotide positions 1, 2, 3, 4, 5, 6, 7, 8, 9, 10, 11, 12, 13, 14, or 15 increases activity, e.g., relative to a reference sequence, e.g., increases by about 1.5-fold, about 2-fold, or about 2.5-fold, e.g., as shown in FIG. 9. In an embodiment, the activity of a TREM comprising a Glu-TAG scaffold, e.g., a TREM corresponding to a reference sequence, e.g., SEQ ID NO: 1867, 2000, or 2001, comprising a nucleotide substitution at any of nucleotide positions 20a, 21, 22, 23, 24, 25, 26, 27, 28, 29, 30, or 31 is increased, e.g., relative to a reference sequence, e.g., increased by about 1.5-fold, about 2-fold, about 2.5-fold, or about 3-fold, e.g., as shown in FIG. 9. In an embodiment, the activity of a TREM comprising a Glu-TAG scaffold, e.g., a TREM corresponding to a reference sequence, e.g., SEQ ID NO: 1867, 2000, or 2001, comprising a nucleotide substitution at any of nucleotide positions 32, 33, 34, 35, 36, 37, or 38 is decreased, e.g., relative to a reference sequence, e.g., decreased by about 2-fold, e.g., as shown in FIG. 9. In an embodiment, the activity of a TREM comprising a Glu-TAG scaffold, e.g., a TREM corresponding to a reference sequence, e.g., SEQ ID NO: 1867, 2000, or 2001, comprising a nucleotide substitution at any of nucleotide positions 40, 41, 42, 43, 44, or 45 is increased, e.g., relative to a reference sequence, e.g., increased by about 1.5-fold or about 2-fold, e.g., as shown in FIG. 9. In an embodiment, the activity of a TREM comprising a Glu-TAG scaffold, e.g., a TREM corresponding to a reference sequence, e.g., SEQ ID NO: 1867, 2000, or 2001, comprising a nucleotide substitution at any of nucleotide positions 46 or 48 is increased, e.g., relative to a reference sequence, e.g., increased by about 1.5-fold or about 2-fold, e.g., as shown in FIG. 9. In an embodiment, the activity of a TREM comprising a Glu-TAG scaffold, e.g., a TREM corresponding to a reference sequence, e.g., SEQ ID NO: 1867, 2000, or 2001, comprising a nucleotide substitution at any of nucleotide positions 59 or 60 is increased, e.g., relative to a reference sequence, e.g., increased by about 1.5-fold, e.g., as shown in FIG. 9. In an embodiment, the activity of a TREM comprising a Glu-TAG scaffold, e.g., a TREM corresponding to a reference sequence, e.g., SEQ ID NO: 1867, 2000, or 2001, comprising a nucleotide substitution at any of nucleotide positions 66, 67, 68, 69, 70, 71, 72, or 73 is increased, e.g., relative to a reference sequence, e.g., increased by about 1.5-fold, about 2-fold, or about 2.5-fold, e.g., as shown in FIG. 9.

In an embodiment, a TREM comprising a Glu-TAG scaffold, e.g., a TREM corresponding to a reference sequence, e.g., SEQ ID NO: 1867, comprises a nucleotide substitution at any one of the positions shown in FIG. 10. In an embodiment, a TREM comprising a Glu-TAG scaffold, e.g., a TREM corresponding to a reference sequence, e.g., SEQ ID NO: 1867, comprises a nucleotide substitution at any of nucleotide positions 1, 2, 3, 4, 5, 6, 7, 8, 9, 10, 11, 12, 13, 14, or 15, e.g., in the [ASt Domain1] or the [DH Domain], e.g., as shown in FIG. 10. In an embodiment, a TREM comprising a Glu-TAG scaffold, e.g., a TREM corresponding to a reference sequence, e.g., SEQ ID NO: 1867, comprises a nucleotide substitution at any of nucleotide positions 20a, 21, 22, 23, 24, 25, 26, 27, 28, 29, 30, or 31, e.g., in the [DH Domain] or the [ACH Domain], e.g., as shown in FIG. 10. In an embodiment, a TREM comprising a Glu-TAG scaffold, e.g., a TREM corresponding to a reference sequence, e.g., SEQ ID NO: 1867, comprises a nucleotide substitution at any of nucleotide positions 32, 33, 34, 35, 36, 37, or 38, e.g., in the [ACH Domain], e.g., as shown in FIG. 10. In an embodiment, a TREM comprising a Glu-TAG scaffold, e.g., a TREM corresponding to a reference sequence, e.g., SEQ ID NO: 1867, comprises a nucleotide substitution at any of nucleotide positions 40, 41, 42, 43, 44, or 45, e.g., in the [ACH Domain], e.g., as shown in FIG. 10. In an embodiment, a TREM comprising a Glu-TAG scaffold, e.g., a TREM corresponding to a reference sequence, e.g., SEQ ID NO: 1867, comprises a nucleotide substitution at any of nucleotide positions 46, 48, or 49, e.g., in the [VL Domain], e.g., as shown in FIG. 10. In an embodiment, a TREM comprising a Glu-TAG scaffold, e.g., a TREM corresponding to a reference sequence, e.g., SEQ ID NO: 1867, comprises a nucleotide substitution at any of nucleotide positions 59 or 60, e.g., in the [TH Domain], e.g., as shown in FIG. 10. In an embodiment, a TREM comprising a Glu-TAG scaffold, e.g., a TREM corresponding to a reference sequence, e.g., SEQ ID NO: 1867, comprises a nucleotide substitution at any of nucleotide positions 66, 67, 68, 69, 70, 71, 72, or 73, e.g., in the [ASt Domain2], e.g., as shown in FIG. 10. In an embodiment, the activity of a TREM comprising a Glu-TAG scaffold, e.g., a TREM corresponding to a reference sequence, e.g., SEQ ID NO: 1867, comprising a nucleotide substitution at any of nucleotide positions 1, 2, 3, 4, 5, 6, 7, 8, 9, 10, 11, 12, 13, 14, or 15 increases activity, e.g., relative to a reference sequence, e.g., increases by about 1.5-fold, about 2-fold, about 2.5-fold, or about 3-fold, e.g., as shown in FIG. 10. In an embodiment, the activity of a TREM comprising a Glu-TAG scaffold, e.g., a TREM corresponding to a reference sequence, e.g., SEQ ID NO: 1867, comprising a nucleotide substitution at any of nucleotide positions 20a, 21, 22, 23, 24, 25, 26, 27, 28, 29, 30, or 31 is increased, e.g., relative to a reference sequence, e.g., increased by about 1.5-fold, about 2-fold, about 2.5-fold, or about 3-fold, e.g., as shown in FIG. 10. In an embodiment, the activity of a TREM comprising a Glu-TAG scaffold, e.g., a TREM corresponding to a reference sequence, e.g., SEQ ID NO: 1867, comprising a nucleotide substitution at any of nucleotide positions 32, 33, 34, 35, 36, 37, or 38 is decreased, e.g., relative to a reference sequence, e.g., decreased by about 2-fold, e.g., as shown in FIG. 10. In an embodiment, the activity of a TREM comprising a Glu-TAG scaffold, e.g., a TREM corresponding to a reference sequence, e.g., SEQ ID NO: 1867, comprising a nucleotide substitution at any of nucleotide positions 40, 41, 42, 43, 44, or 45 is increased, e.g., relative to a reference sequence, e.g., increased by about 1.5-fold or about 2-fold, e.g., as shown in FIG. 10. In an embodiment, the activity of a TREM comprising a Glu-TAG scaffold, e.g., a TREM corresponding to a reference sequence, e.g., SEQ ID NO: 1867, comprising a nucleotide substitution at any of nucleotide positions 46 or 48 is increased, e.g., relative to a reference sequence, e.g., increased by about 1.5-fold or about 2-fold, e.g., as shown in FIG. 10. In an embodiment, the activity of a TREM comprising a Glu-TAG scaffold, e.g., a TREM corresponding to a reference sequence, e.g., SEQ ID NO: 1867, comprising a nucleotide substitution at any of nucleotide positions 59 or 60 is increased, e.g., relative to a reference sequence, e.g., increased by about 1.5-fold, e.g., as shown in FIG. 10. In an embodiment, the activity of a TREM comprising a Glu-TAG scaffold, e.g., a TREM corresponding to a reference sequence, e.g., SEQ ID NO: 1867, comprising a nucleotide substitution at any of nucleotide positions 66, 67, 68, 69, 70, 71, 72, or 73 is increased, e.g., relative to a reference sequence, e.g., increased by about 1.5-fold, or about 2-fold, e.g., as shown in FIG. 10.

In an embodiment, a TREM comprising a Glu-TAG scaffold, e.g., a TREM corresponding to a reference sequence, e.g., SEQ ID NO: 2000, comprises a nucleotide substitution at any one of the positions shown in FIG. 11. In an embodiment, a TREM comprising a Glu-TAG scaffold, e.g., a TREM corresponding to a reference sequence, e.g., SEQ ID NO: 2000, comprises a nucleotide substitution at any of nucleotide positions 31 or 32, e.g., in the [ACH Domain], e.g., as shown in FIG. 11. In an embodiment, a TREM comprising a Glu-TAG scaffold, e.g., a TREM corresponding to a reference sequence, e.g., SEQ ID NO: 2000, comprises a nucleotide substitution at any of nucleotide positions 32, 33, 34, 35, 36, 37, or 38, e.g., in the [ACH Domain], e.g., as shown in FIG. 11. In an embodiment, the activity of a TREM comprising a Glu-TAG scaffold, e.g., a TREM corresponding to a reference sequence, e.g., SEQ ID NO: 2000, comprising a nucleotide substitution at any of nucleotide positions 31 or 32 is increased, e.g., relative to a reference sequence, e.g., increased by about 5-fold or about 11-fold, e.g., as shown in FIG. 11. In an embodiment, the activity of a TREM comprising a Glu-TAG scaffold, e.g., a TREM corresponding to a reference sequence, e.g., SEQ ID NO: 2000, comprising a nucleotide substitution at any of nucleotide positions 32, 33, 34, 35, 36, 37, or 38 is decreased, e.g., relative to a reference sequence, e.g., decreased by about 2-fold, e.g., as shown in FIG. 11.

In an embodiment, a TREM comprising a Leu-TAG scaffold, e.g., a TREM corresponding to a reference sequence, e.g., SEQ ID NO: 2016 or 2017, comprises a nucleotide substitution at any one of the positions shown in FIG. 12. In an embodiment, a TREM comprising a Leu-TAG scaffold, e.g., a TREM corresponding to a reference sequence, e.g., SEQ ID NO: 2016 or 2017, comprises a nucleotide substitution at any of nucleotide positions 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30, e.g., in the [ASt Domain1], the [DH Domain], or the [ACH Domain], e.g., as shown in FIG. 12. In an embodiment, a TREM comprising a Leu-TAG scaffold, e.g., a TREM corresponding to a reference sequence, e.g., SEQ ID NO: 2016 or 2017, comprises a nucleotide substitution at any of nucleotide positions 32, 33, 34, 35, 36, 37, or 38, e.g., in the [ACH Domain], e.g., as shown in FIG. 12. In an embodiment, a TREM comprising a Leu-TAG scaffold, e.g., a TREM corresponding to a reference sequence, e.g., SEQ ID NO: 2016 or 2017, comprises a nucleotide substitution at any of nucleotide positions 39, 40, 41, 42, 43, 44, 45, Vi1, V12, V13, V14, V15, V16, V17, V1, V2, V3, V4, V5, V27, V26, V25, V24, V23, V22, V21, 46, 47, 48, 49, or 50, e.g., in the [ACH Domain], the [VL Domain], or the [TH Domain], e.g., as shown in FIG. 12. In an embodiment, a TREM comprising a Leu-TAG scaffold, e.g., a TREM corresponding to a reference sequence, e.g., SEQ ID NO: 2016 or 2017, comprises a nucleotide substitution at nucleotide positions 59, e.g., in the [TH Domain], e.g., as shown in FIG. 12. In an embodiment, a TREM comprising a Leu-TAG scaffold, e.g., a TREM corresponding to a reference sequence, e.g., SEQ ID NO: 2016 or 2017, comprises a nucleotide substitution at any of nucleotide positions 64, 65, 66, 67, 68, 69, 70, 71, 72, or 73, e.g., in the [TH Domain] or the [ASt Domain2], e.g., as shown in FIG. 12. In an embodiment, the activity of a TREM comprising a Leu-TAG scaffold, e.g., a TREM corresponding to a reference sequence, e.g., SEQ ID NO: 2016 or 2017, comprising a nucleotide substitution at any of nucleotide positions 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 is increased, e.g., relative to a reference sequence, e.g., increased by about 1.5-fold or about 2-fold, e.g., as shown in FIG. 12. In an embodiment, the activity of a TREM comprising a Leu-TAG scaffold, e.g., a TREM corresponding to a reference sequence, e.g., SEQ ID NO: 2016 or 2017, comprising a nucleotide substitution at any of nucleotide positions 32, 33, 34, 35, 36, 37, or 38 is decreased, e.g., relative to a reference sequence, e.g., decreased by about 2-fold, e.g., as shown in FIG. 12. In an embodiment, the activity of a TREM comprising a Leu-TAG scaffold, e.g., a TREM corresponding to a reference sequence, e.g., SEQ ID NO: 2016 or 2017, comprising a nucleotide substitution at any of nucleotide positions 39, 40, 41, 42, 43, 44, 45, V11, V12, V13, V14, V15, V16, V17, V1, V2, V3, V4, V5, V27, V26, V25, V24, V23, V22, V21, 46, 47, 48, 49, or 50 is increased, e.g., relative to a reference sequence, e.g., increased by about 1.5-fold or about 2-fold, e.g., as shown in FIG. 12. In an embodiment, the activity of a TREM comprising a Leu-TAG scaffold, e.g., a TREM corresponding to a reference sequence, e.g., SEQ ID NO: 2016 or 2017, comprising a nucleotide substitution at nucleotide position 59 is increased, e.g., relative to a reference sequence, e.g., increased by about 1.5-fold or about 2-fold, e.g., as shown in FIG. 12. In an embodiment, the activity of a TREM comprising a Leu-TAG scaffold, e.g., a TREM corresponding to a reference sequence, e.g., SEQ ID NO: 2016 or 2017, comprising a nucleotide substitution at any of nucleotide positions 64, 65, 66, 67, 68, 69, 70, 71, 72, or 73 is increased, e.g., relative to a reference sequence, e.g., increased by about 1.5-fold or about 2-fold, e.g., as shown in FIG. 12.

In an embodiment, a TREM comprising a Tyr-TAG scaffold, e.g., a TREM corresponding to a reference sequence, e.g., SEQ ID NO: 2022 or 2023, comprises a nucleotide substitution at any one of the positions shown in FIG. 13. In an embodiment, a TREM comprising a Tyr-TAG scaffold, e.g., a TREM corresponding to a reference sequence, e.g., SEQ ID NO: 2022 or 2023, comprises a nucleotide substitution at any of nucleotide positions 4, 5, 6, or 7, e.g., in the [ASt Domain1], e.g., as shown in FIG. 13. In an embodiment, a TREM comprising a Tyr-TAG scaffold, e.g., a TREM corresponding to a reference sequence, e.g., SEQ ID NO: 2022 or 2023, comprises a nucleotide substitution at any of nucleotide positions 16, 17, or 20, e.g., in the [DH Domain], e.g., as shown in FIG. 13. In an embodiment, a TREM comprising a Tyr-TAG scaffold, e.g., a TREM corresponding to a reference sequence, e.g., SEQ ID NO: 2022 or 2023, comprises a nucleotide substitution at any of nucleotide positions 28, 29, or 30, e.g., in the [ACH Domain], e.g., as shown in FIG. 13. In an embodiment, a TREM comprising a Tyr-TAG scaffold, e.g., a TREM corresponding to a reference sequence, e.g., SEQ ID NO: 2022 or 2023, comprises a nucleotide substitution at any of nucleotide positions 32, 33, 34, 35, 36, 37, or 38, e.g., in the [ACH Domain], e.g., as shown in FIG. 13. In an embodiment, a TREM comprising a Tyr-TAG scaffold, e.g., a TREM corresponding to a reference sequence, e.g., SEQ ID NO: 2022 or 2023, comprises a nucleotide substitution at any of nucleotide positions 40, 41, 42, 43, or 44, e.g., in the [ACH Domain], e.g., as shown in FIG. 13. In an embodiment, a TREM comprising a Tyr-TAG scaffold, e.g., a TREM corresponding to a reference sequence, e.g., SEQ ID NO: 2022 or 2023, comprises a nucleotide substitution at any of nucleotide positions 47, 48, 49, 50, or 51, e.g., in the [VL Domain] or the [TH Domain], e.g., as shown in FIG. 13. In an embodiment, a TREM comprising a Tyr-TAG scaffold, e.g., a TREM corresponding to a reference sequence, e.g., SEQ ID NO: 2022 or 2023, comprises a nucleotide substitution at any of nucleotide positions 59, 60, 61, 62, 63, 64, 65, 66, 67, 68, 69, or 70, e.g., in the [TH Domain] or the [ASt Domain2], e.g., as shown in FIG. 13. In an embodiment, the activity of a TREM comprising a Tyr-TAG scaffold, e.g., a TREM corresponding to a reference sequence, e.g., SEQ ID NO: 2022 or 2023, comprising a nucleotide substitution at any of nucleotide positions 4, 5, 6, or 7 is increased, e.g., relative to a reference sequence, e.g., increased by about 1.5-fold, about 2-fold, or about 2.5-fold, e.g., as shown in FIG. 13. In an embodiment, the activity of a TREM comprising a Tyr-TAG scaffold, e.g., a TREM corresponding to a reference sequence, e.g., SEQ ID NO: 2022 or 2023, comprising a nucleotide substitution at any of nucleotide positions 16, 17, or 20 is increased, e.g., relative to a reference sequence, e.g., increased by about 1.5-fold or about 2-fold, e.g., as shown in FIG. 13. In an embodiment, the activity of a TREM comprising a Tyr-TAG scaffold, e.g., a TREM corresponding to a reference sequence, e.g., SEQ ID NO: 2022 or 2023, comprising a nucleotide substitution at any of nucleotide positions 28, 29, or 30 is increased, e.g., relative to a reference sequence, e.g., increased by about 1.5-fold, about 2-fold, or about 2.5-fold, e.g., as shown in FIG. 13. In an embodiment, the activity of a TREM comprising a Tyr-TAG scaffold, e.g., a TREM corresponding to a reference sequence, e.g., SEQ ID NO: 2022 or 2023, comprising a nucleotide substitution at any of nucleotide positions 32, 33, 34, 35, 36, 37, or 38 is decreased, e.g., relative to a reference sequence, e.g., decreased by about 2-fold, e.g., as shown in FIG. 13. In an embodiment, the activity of a TREM comprising a Tyr-TAG scaffold, e.g., a TREM corresponding to a reference sequence, e.g., SEQ ID NO: 2022 or 2023, comprising a nucleotide substitution at any of nucleotide positions 40, 41, 42, 43, or 44 is increased, e.g., relative to a reference sequence, e.g., increased by about 1.5-fold, about 2-fold, or about 2.5-fold, e.g., as shown in FIG. 13. In an embodiment, the activity of a TREM comprising a Tyr-TAG scaffold, e.g., a TREM corresponding to a reference sequence, e.g., SEQ ID NO: 2022 or 2023, comprising a nucleotide substitution at any of nucleotide positions 47, 48, 49, 50, or 51 is increased, e.g., relative to a reference sequence, e.g., increased by about 1.5-fold, about 2-fold, or about 2.5-fold, e.g., as shown in FIG. 13. In an embodiment, the activity of a TREM comprising a Tyr-TAG scaffold, e.g., a TREM corresponding to a reference sequence, e.g., SEQ ID NO: 2022 or 2023, comprising a nucleotide substitution at any of nucleotide positions 59, 60, 61, 62, 63, 64, 65, 66, 67, 68, 69, or 70 is increased, e.g., relative to a reference sequence, e.g., increased by about 1.5-fold, about 2-fold, or about 2.5-fold, e.g., as shown in FIG. 13.

In an embodiment, a TREM comprising a Ser-TAG scaffold, e.g., a TREM corresponding to a reference sequence, e.g., SEQ ID NO: 2020 or 2021, comprises a nucleotide substitution at any one of the positions shown in FIG. 14. In an embodiment, a TREM comprising a Ser-TAG scaffold, e.g., a TREM corresponding to a reference sequence, e.g., SEQ ID NO: 2020 or 2021, comprises a nucleotide substitution at any of nucleotide positions 3, 4, 5, or 6, e.g., in the [ASt Domain1], e.g., as shown in FIG. 14. In an embodiment, a TREM comprising a Ser-TAG scaffold, e.g., a TREM corresponding to a reference sequence, e.g., SEQ ID NO: 2020 or 2021, comprises a nucleotide substitution at any of nucleotide positions 29, 30 31, 32, or 33, e.g., in the [ACH Domain], e.g., as shown in FIG. 14. In an embodiment, a TREM comprising a Ser-TAG scaffold, e.g., a TREM corresponding to a reference sequence, e.g., SEQ ID NO: 2020 or 2021, comprises a nucleotide substitution at any of nucleotide positions 34, 35, or 36, e.g., in the [ACH Domain], e.g., as shown in FIG. 14. In an embodiment, a TREM comprising a Ser-TAG scaffold, e.g., a TREM corresponding to a reference sequence, e.g., SEQ ID NO: 2020 or 2021, comprises a nucleotide substitution at any of nucleotide positions 37, 38, 39, 40, or 41, e.g., in the [ACH Domain], e.g., as shown in FIG. 14. In an embodiment, a TREM comprising a Ser-TAG scaffold, e.g., a TREM corresponding to a reference sequence, e.g., SEQ ID NO: 2020 or 2021, comprises a nucleotide substitution at nucleotide position 51, e.g., in the [TH Domain], e.g., as shown in FIG. 14. In an embodiment, a TREM comprising a Ser-TAG scaffold, e.g., a TREM corresponding to a reference sequence, e.g., SEQ ID NO: 2020 or 2021, comprises a nucleotide substitution at nucleotide position 63, e.g., in the [TH Domain], e.g., as shown in FIG. 14. In an embodiment, a TREM comprising a Ser-TAG scaffold, e.g., a TREM corresponding to a reference sequence, e.g., SEQ ID NO: 2020 or 2021, comprises a nucleotide substitution at nucleotide position 67, e.g., in the [ASt Domain2], e.g., as shown in FIG. 14. In an embodiment, the activity of a TREM comprising a Ser-TAG scaffold, e.g., a TREM corresponding to a reference sequence, e.g., SEQ ID NO: 2020 or 2021, comprising a nucleotide substitution at any of nucleotide positions 3, 4, 5, or 6 is increased, e.g., relative to a reference sequence, e.g., increased by about 2-fold or about 3-fold, e.g., as shown in FIG. 14. In an embodiment, the activity of a TREM comprising a Ser-TAG scaffold, e.g., a TREM corresponding to a reference sequence, e.g., SEQ ID NO: 2020 or 2021, comprising a nucleotide substitution at any of nucleotide positions 29, 30 31, 32, or 33 is increased, e.g., relative to a reference sequence, e.g., increased by about 1.5-fold or about 2-fold, e.g., as shown in FIG. 14. In an embodiment, the activity of a TREM comprising a Ser-TAG scaffold, e.g., a TREM corresponding to a reference sequence, e.g., SEQ ID NO: 2020 or 2021, comprising a nucleotide substitution at any of nucleotide positions 34, 35, or 36 is decreased, e.g., relative to a reference sequence, e.g., decreased by about 2-fold, e.g., as shown in FIG. 14. In an embodiment, the activity of a TREM comprising a Ser-TAG scaffold, e.g., a TREM corresponding to a reference sequence, e.g., SEQ ID NO: 2020 or 2021, comprising a nucleotide substitution at any of nucleotide positions 37, 38, 39, 40, or 41 is increased, e.g., relative to a reference sequence, e.g., increased by about 1.5-fold, about 2-fold, about 2.5-fold, or about 3-fold, e.g., as shown in FIG. 14. In an embodiment, the activity of a TREM comprising a Ser-TAG scaffold, e.g., a TREM corresponding to a reference sequence, e.g., SEQ ID NO: 2020 or 2021, comprising a nucleotide substitution at nucleotide position 51 is increased, e.g., relative to a reference sequence, e.g., increased by about 4-fold, e.g., as shown in FIG. 14. In an embodiment, the activity of a TREM comprising a Ser-TAG scaffold, e.g., a TREM corresponding to a reference sequence, e.g., SEQ ID NO: 2020 or 2021, comprising a nucleotide substitution at nucleotide position 63 is increased, e.g., relative to a reference sequence, e.g., increased by about 4-fold, e.g., as shown in FIG. 14. In an embodiment, the activity of a TREM comprising a Ser-TAG scaffold, e.g., a TREM corresponding to a reference sequence, e.g., SEQ ID NO: 2020 or 2021, comprising a nucleotide substitution at nucleotide position 67 is increased, e.g., relative to a reference sequence, e.g., increased by about 3-fold, e.g., as shown in FIG. 14.

In an embodiment, a TREM comprising a Lys-TAG scaffold, e.g., a TREM corresponding to a reference sequence, e.g., SEQ ID NO: 2018 or 2019, comprises a nucleotide substitution at any one of the positions shown in FIG. 15. In an embodiment, a TREM comprising a Lys-TAG scaffold, e.g., a TREM corresponding to a reference sequence, e.g., SEQ ID NO: 2018 or 2019, comprises a nucleotide substitution at any of nucleotide positions 1, 2, 3, 4, 5, 6, or 7, e.g., in the [ASt Domain1], e.g., as shown in FIG. 15. In an embodiment, a TREM comprising a Lys-TAG scaffold, e.g., a TREM corresponding to a reference sequence, e.g., SEQ ID NO: 2018 or 2019, comprises a nucleotide substitution at any of nucleotide positions 17 or 18, e.g., in the [DH Domain], e.g., as shown in FIG. 15. In an embodiment, a TREM comprising a Lys-TAG scaffold, e.g., a TREM corresponding to a reference sequence, e.g., SEQ ID NO: 2018 or 2019, comprises a nucleotide substitution at any of nucleotide positions 32, 33, 34, 35, 36, 37, or 38, e.g., in the [ACH Domain], e.g., as shown in FIG. 15. In an embodiment, a TREM comprising a Lys-TAG scaffold, e.g., a TREM corresponding to a reference sequence, e.g., SEQ ID NO: 2018 or 2019, comprises a nucleotide substitution at nucleotide position 37, e.g., in the [ACH Domain], e.g., as shown in FIG. 15. In an embodiment, a TREM comprising a Lys-TAG scaffold, e.g., a TREM corresponding to a reference sequence, e.g., SEQ ID NO: 2018 or 2019, comprises a nucleotide substitution at nucleotide position 50, e.g., in the [TH Domain], e.g., as shown in FIG. 15. In an embodiment, a TREM comprising a Lys-TAG scaffold, e.g., a TREM corresponding to a reference sequence, e.g., SEQ ID NO: 2018 or 2019, comprises a nucleotide substitution at any of nucleotide positions 64, 65, 66, 67, 68, 69, 70, 71, 72, or 73, e.g., in the [DH Domain] or the [ASt Domain2], e.g., as shown in FIG. 15. In an embodiment, the activity of a TREM comprising a Lys-TAG scaffold, e.g., a TREM corresponding to a reference sequence, e.g., SEQ ID NO: 2018 or 2019, comprising a nucleotide substitution at any of nucleotide positions 1, 2, 3, 4, 5, 6, or 7 is increased, e.g., relative to a reference sequence, e.g., increased by about 1.5-fold, about 2-fold, or about 4-fold, e.g., as shown in FIG. 15. In an embodiment, the activity of a TREM comprising a Lys-TAG scaffold, e.g., a TREM corresponding to a reference sequence, e.g., SEQ ID NO: 2018 or 2019, comprising a nucleotide substitution at any of nucleotide positions 17 or 18 is increased, e.g., relative to a reference sequence, e.g., increased by about 2-fold or about 3-fold, e.g., as shown in FIG. 15. In an embodiment, the activity of a TREM comprising a Lys-TAG scaffold, e.g., a TREM corresponding to a reference sequence, e.g., SEQ ID NO: 2018 or 2019, comprising a nucleotide substitution at any of nucleotide positions 32, 33, 34, 35, 36, 37, or 38 is decreased, e.g., relative to a reference sequence, e.g., decreased by about 2-fold, e.g., as shown in FIG. 15. In an embodiment, the activity of a TREM comprising a Lys-TAG scaffold, e.g., a TREM corresponding to a reference sequence, e.g., SEQ ID NO: 2018 or 2019, comprising a nucleotide substitution at nucleotide position 37 is increased, e.g., relative to a reference sequence, e.g., increased by about 3-fold, e.g., as shown in FIG. 15. In an embodiment, the activity of a TREM comprising a Lys-TAG scaffold, e.g., a TREM corresponding to a reference sequence, e.g., SEQ ID NO: 2018 or 2019, comprising a nucleotide substitution at nucleotide position 50 is increased, e.g., relative to a reference sequence, e.g., increased by about 3-fold, e.g., as shown in FIG. 15. In an embodiment, the activity of a TREM comprising a Lys-TAG scaffold, e.g., a TREM corresponding to a reference sequence, e.g., SEQ ID NO: 2018 or 2019, comprising a nucleotide substitution at any of nucleotide positions 64, 65, 66, 67, 68, 69, 70, 71, 72, or 73 is increased, e.g., relative to a reference sequence, e.g. increased by about 1.5-fold, about 2-fold, about 2.5-fold, about 3.5-fold, or about 4-fold, e.g., as shown in FIG. 15.

### Non-Naturally Occurring Modification

A TREM, a TREM core fragment or a TREM fragment described herein may comprise a non-naturally occurring modification, e.g., a modification described in Table 5. A non-naturally occurring modification can be made according to methods known in the art. In an embodiment, a non-naturally occurring modification is a modification that a cell, e.g., a human cell, does not make on an endogenous tRNA. In an embodiment, a non-naturally occurring modification is a modification that a cell, e.g., a human cell, can make on an endogenous tRNA, but wherein such modification is in a location in which it does not occur on a native tRNA. In an embodiment, the non-naturally occurring modification is in a domain, linker or arm which does not have such modification in nature. In an embodiment, the non-naturally occurring modification is at a position within a domain, linker or arm, which does not have such modification in nature. In an embodiment, the non-naturally occurring modification is on a nucleotide which does not have such modification in nature. In an embodiment, the non-naturally occurring modification is on a nucleotide at a position within a domain, linker or arm, which does not have such modification in nature.

In an embodiment, a TREM, a TREM core fragment or a TREM fragment described herein comprises a modification provided in Table 5, or a combination thereof. The modifications provided in Table 5 are non-naturally occurring or occur naturally in RNAs, and are used herein on a synthetic TREM, a TREM core fragment or a TREM fragment at a position that does not occur in nature.

TABLE 5
Exemplary modifications
Chemical Modification

| | |
|---|---|
| (S)-constrained ethyl (cEt) | 5-(methoxycarbonyl-methyl)uracil |
| (±)1-(2-Hydroxypropyl)pseudouridine | 5-(methyl) 2(thio)uracil |
| (2R)-1-(2-Hydroxypropyl)pseudouridine | 5-(methyl) 2,4 (dithio)uracil |
| (2S)-1-(2-Hydroxypropyl)pseudouridine | 5-(methyl) 4 (thio)uracil |
| (3-(3-amino-3-carboxypropyl)uridine | 5-(methyl)-2-(thio)pseudouracil |
| (E)-5-(2-Bromo-vinyl)ara-uridine | 5-(methyl)-2-(thio)uracil |
| (E)-5-(2-Bromo-vinyl)cytidine | 5-(methyl)-2,4 (dithio)pseudouracil |
| (E)-5-(2-Bromo-vinyl)uridine | 5-(methyl)-2,4-(dithio)uracil |
| (E)-vinylphosphonate | 5-(methyl)-4 (thio)pseudouracil |
| (R) 5′-C-methyl | 5-(methyl)isocarbostyrilyl |
| (R) 5′-C-methyl with phosphate | 5-(methyl)pseudouracil |
| (S) 5′-C-methyl | 5-(methylaminomethyl)-2 (thio)uracil |
| (S) 5′-C-methyl with phosphate | 5-(methylaminomethyl)-2,4(dithio)uracil |
| (Z)-5-(2-Bromo-vinyl)ara-uridine | 5-(methylaminomethyl)-4-(thio)uracil |
| (Z)-5-(2-Bromo-vinyl)uridine | 5-(propynyl)uracil |
| 1(4-Nitro-phenyl)pseudouridine | 5-(propynyl)cytosine |
| 1-(aminocarbonylethylenyl)-2(thio)- | 5-(trifluoromethyl)cytosine |
| pseudouracil | |
| 1-(aminocarbonylethylenyl)-2,4- | 5-(trifluoromethyl)uracil |
| (dithio)pseudouracil | |
| 1-(2,2,2-Trifluoroethyl)-pseudouridine | 5,2′-O-dimethylcytidine |
| 1-(2,2,3,3,3-Pentafluoropropyl)pseudouridine | 5,2′-O-dimethyluridine |
| 1-(2,2-Diethoxyethyl)pseudouridine | 5,6-dihydro-uridine |
| 1-(2,4,6-Trimethylbenzyl)pseudouridine | 5-Aminoallyl-cytosine |
| 1-(2,4,6-Trimethyl-benzyl)pseudo-uridine | 5-aminoallyl-uridine |
| 1-(2,4,6-Trimethyl-phenyl)pseudo-uridine | 5-aminomethyl-2-thiouridine |
| 1-(2-Amino-2-carboxyethyl)pseudo-uridine | 5-aza-2-thio-zebularine |
| 1-(2-Amino-ethyl)pseudouridine | 5-aza-cytidine |
| 1-(2-Hydroxyethyl)pseudouridine | 5-aza-uridine |
| 1-(2-Methoxyethyl)pseudouridine | 5-aza-zebularine |
| 1-(3,4-Bis- | 5-bromo-cytidine |
| trifluoromethoxvbenzyl)pseudouridine | |
| 1-(3,4-Dimethoxybenzyl)pseudouridine | 5-bromo-uridine |
| 1-(3-Amino-3-carboxypropyl)pseudo-uridine | 5-carbamoylmethyl-2′-O-methyluridine |
| 1-(3-Amino-propyl)pseudouridine | 5-carbamoylmethyluridine |
| 1-(3-Cyclopropyl-prop-2-ynyl)pseudouridine | 5-carboxyhydroxymethyluridine |
| TP | |
| 1-(4-Amino-4-carboxybutyl)pseudouridine | 5-carboxyhydroxymethyluridine methyl ester |
| 1-(4-Amino-benzyl)pseudouridine | 5-carboxymethylaminomethyl-2-thiouridine |
| 1-(4-Amino-butyl)pseudouridine | 5-carboxymethylaminomethyl-2′-O- |
| | methyluridine |
| 1-(4-Amino-phenyl)pseudouridine | 5-carboxymethylaminomethyl-2-thiouridine |
| 1-(4-Azidobenzyl)pseudouridine | 5-carboxymethylaminomethyluridine |
| 1-(4-Bromobenzyl)pseudouridine | 5-carboxymethyluridine |
| 1-(4-Chlorobenzyl)pseudouridine | 5-Cyanocytidine |
| 1-(4-Fluorobenzyl)pseudouridine | 5-Cyanouridine |
| 1-(4-Iodobenzyl)pseudouridine | 5-Dimethylaminouridine |
| 1-(4-Methanesulfonylbenzyl)pseudouridine | 5-Ethynylara-cytidine |
| 1-(4-Methoxybenzyl)pseudouridine | 5-Ethynylcytidine |
| 1-(4-Methoxy-phenyl)pseudouridine | 5-formyl-2′-O-methylcytidine |
| 1-(4-Methylbenzyl)pseudouridine | 5-formylcytidine |
| 1-(4-Nitrobenzyl)pseudouridine | 5′-Homo-adenosine |
| 1-(4-Thiomethoxybenzyl)pseudouridine | 5′-Homo-cytidine |
| 1-(4-Trifluoromethoxybenzyl)pseudouridine | 5′-Homo-guanosine |
| 1-(4-Trifluoromethylbenzyl)pseudouridine | 5′-Homo-uridine |
| 1-(5-Amino-pentyl)pseudouridine | 5-hydroxymethylcytidine |
| 1-(6-Amino-hexyl)pseudouridine | 5-hydroxyuridine |
| 1-(aminoalkylamino-carbonylethylenyl)- | 5-iodo-2′-fluoro-deoxyuridine |
| 2(thio)-pseudouracil | |
| 1-(aminoalkylaminocarbonylethylenyl)-2,4- | 5-iodo-cytidine |
| (dithio)pseudouracil | |
| 1-(aminoalkylaminocarbonylethylenyl)- | 5-iodo-uridine |
| pseudouracil | |
| 1-(aminoalkylaminocarbonylethylenyl)-4- | 5-methoxycarbonylmethy1-2-thiouridine |
| (thio)pseudouracil | |
| 1-(aminocarbonylethylenyl)-4- | 5-methoxycarbonylmethyl-2′-O- |
| (thio)pseudouracil | methyluridine |
| 1-(aminocarbonylethylenyl)-pseudouracil | 5-methoxycarbonylmethyluridine |
| 1-(aza)-2-(thio)-3-(aza)-phenoxazin-1-yl | 5-Methoxycytidine |
| 1,2′-O-dimethyladenosine | 5-methoxyuridine |
| 1,2′-O-dimethylguanosine | 5-methyl-2-thiouridine |
| 1,2′-O-dimethylinosine | 5-methylaminomethyl-2-selenouridine |
| 1,3-(diaza)-2-(oxo)-phenthiazin-1-yl | 5-methylaminomethyl-2-thiouridine |
| 1,3-(diaza)-2-(oxo)-phenoxazin-1-yl | 5-methylaminomethyluridine |
| 1,3,5-(triaza)-2,6-(dioxa)-naphthalene | 5-methylcytidine |
| 1,6-Dimethyl-pseudouridine | 5-Methyldihydrouridine |
| 1-{3-[2-(2-Aminoethoxy)-ethoxy]- | 5-methyluridine |
| propionvl}pseudouridine | |
| 1-Acetylpseudouridine | 5-methyl-zebularine |
| 1-Allylpseudouridine | 5-nitroindole |
| 1-Aminomethyl-pseudo-uridine | 5-Oxyacetic acid-Uridine |
| 1-Benzoylpseudouridine | 5-Oxyacetic acid-methyl ester-Uridin Nl- |
| | methyl-pseudouridine |
| 1-Benzyloxymethylpseudouridine | 5-Phenylethynyluridine |
| 1-Benzyl-pseudo-uridine | 5′-phosphorothioate |
| 1-Biotinylpseudouridine | 5-propynyl cytosine |
| 1-Butyl-pseudo-uridine | 5-propynyl uracil |
| 1-carboxymethyl-pseudouridine | 5-taurinomethyl-2-thiouridine |
| 1-Cyanomethylpseudouridine | 5-taurinomethyluridine |
| 1-Cyclobutylmethyl-pseudo-uridine | 5-Trideuteromethyl-6-deuterouridine |
| 1-Cyclobutyl-pseudo-uridine | 5-Trifluoromethyl-Cytidine |
| 1-Cycloheptylmethyl-pseudo-uridine | 5-Trifluoromethyl-Uridine |
| 1-Cycloheptyl-pseudo-uridine | 5-uracil |
| 1-Cyclohexylmethyl-pseudo-uridine | 5-Vinylarauridine |
| 1-Cyclohexyl-pseudo-uridine | 6 (azo)uracil |
| 1-Cyclooctylmethyl-pseudo-uridine | 6-(2,2,2-Trifluoroethyl)-pseudo-uridine |
| 1-Cyclooctyl-pseudo-uridine | 6-(4-Morpholino)-pseudo-uridine |
| 1-Cyclopentylmethyl-pseudo-uridine | 6-(4-Thiomorpholino)-pseudo-uridine |
| 1-Cyclopentyl-pseudo-uridine | 6-(alkyl)guanine |
| 1-Cyclopropylmethyl-pseudo-uridine | 6-(alkyl)adenine |
| 1-Cyclopropyl-pseudo-uridine | 6-(aza)pyrimidine |
| 1-deazaadenosine | 6-(azo)cytosine |
| 1-Ethyl-pseudo-uridine | 6-(azo)thymine |
| 1-Hexyl-pseudo-uridine | 6-(azo)uracil |
| 1-Homoallylpseudouridine | 6-(methyl)-7-(aza)indolyl |
| 1-Hydroxymethylpseudouridine | 6-(methyl)adenine |
| 1-iso-propyl-pseudo-uridine | 6-(methyl)guanine |
| 1-Me-2-thio-pseudo-uridine | 6-(Substituted-Phenyl)-pseudo-uridine |
| 1-Me-4-thio-pseudo-uridine | 6-Amino-pseudo-uridine |
| 1-Me-alpha-thio-pseudo-uridine | 6-aza-cytidine |
| 1-Me-guanosine | 6-aza-uridine |
| 1-Methanesulfonylmethylpseudouridine | 6-Azido-pseudo-uridine |
| 1-Methoxymethylpseudouridine | 6-Bromo-pseudo-uridine |
| 1-Methyl-6-amino-pseudo-uridine | 6-Butyl-pseudo-uridine |
| 1-Methyl-6-bromo-pseudo-uridine | 6-Chloro-pseudo-uridine |
| 1-Methyl-6-cyano-pseudo-uridine | 6-chloro-purine |
| 1-Methyl-6-hydroxyamino-pseudo-uridine | 6-Cyano-pseudo-uridine |
| 1-Methyl-6-trifluoromethoxy-pseudo-uridine | 6-Dimethylamino-pseudo-uridine |
| 1-methyladenosine | 6-Ethoxy-pseudo-uridine |
| 1-methylguanosine | 6-Ethylcarboxylate-pseudo-uridine |
| 1-methylinosine | 6-Ethyl-pseudo-uridine |
| 1-methylpseduouridine | 6-Fluoro-pseudo-uridine |
| 1-methyl-pseudoisocytidine | 6-Formyl-pseudo-uridine |
| 1-methyl-pseudouridine | 6-Hydroxyamino-pseudo-uridine |
| 1-Methyl-pseudo-UTP | 6-Hydroxy-pseudo-uridine |
| 1-Morpholinomethylpseudouridine | 6-Iodo-pseudo-uridine |
| 1-Pentyl-pseudo-uridine | 6-iso-Propyl-pseudo-uridine |
| 1-Phenyl-pseudo-uridine | 6-methoxy-guanosine |
| 1-Pivaloylpseudouridine | 6-Methoxy-pseudo-uridine |
| 1-Propargylpseudouridine | 6-Methylamino-pseudo-uridine |
| 1-Propyl-pseudo-uridine | 6-methyl-guanosine |
| 1-propynyl-pseudouridine | 6-Methyl-pseudo-uridine |
| 1-propynyl-uridine | 6-Phenyl-pseudo-uridine |
| 1-p-tolyl-pseudo-uridine | 6-phenyl-pyrrolo-pyrimidin-2-on-3-yl |
| 1-substituted 2-(thio)-pseudouracil | 6-Propyl-pseudo-uridine |
| 1-substituted 2,4-(dithio)pseudouracil | 6-tert-Butyl-pseudo-uridine |
| 1-substituted 4-(thio)pseudouracil | 6-thio-7-deaza-8-aza-guanosine |
| 1-substituted pseudouracil | 6-thio-7-deaza-guanosine |
| 1-taurinomethyl-pseudouridine | 6-thio-7-methyl-guanosine |
| 1-tert-Butyl-pseudo-uridine | 6-thio-guanosine |
| 1-Thiomethoxymethylpseudouridine | 6-Trifluoromethoxy-pseudo-uridine |
| 1-Thiomorpholinomethylpseudouridine | 6-Trifluoromethyl-pseudo-uridine |
| 1-Trifluoroacetylpseudouridine | 7-(alkyl)guanine |
| 1-Trifluoromethylpseudouridine | 7-(aminoalkylhydroxy)-1-(aza)-2-(thio)-3- |
| | (aza)-phenthiazin-1-yl |
| 1-Vinylpseudouridine | 7-(aminoalkylhydroxy)-1-(aza)-2-(thio)-3- |
| | (aza)-phenoxazin-1-yl |
| 2-(amino)purine | 7-(aminoalkylhydroxy)-1,3-(diaza)-2-(oxo)- |
| | phenthiazin-1-yl |
| 2-(thio)pseudouracil | 7-(aminoalkylhydroxy)-1,3-(diaza)-2-(oxo)- |
| | phenoxazin-1-yl |
| 2′-alpha-Ethynylcytidine | 7-(aza)indolyl |
| 2′-alpha-Ethynylguanosine | 7-(deaza)adenine |
| 2′-alpha-Ethynyluridine | 7-(deaza)guanine |
| 2′-alpha-Trifluoromethyladenosine | 7-(guanidiniumalkylhydroxy)-1-(aza)-2- |
| | (thio)-3-(aza)-phenoxazinl-yl |
| 2′-alpha-Trifluoromethylguanosine | 7-(guanidiniumalkylhydroxy)-1-(aza)-2- |
| | (thio)-3-(aza)-phenthiazin-1-yl |
| 2′-alpha-Trifluoromethyluridine | 7-(guanidiniumalkylhydroxy)-1-(aza)-2- |
| | (thio)-3-(aza)-phenoxazin-1-yl |
| 2′-Amino-2′-deoxycytosine | 7-(guanidiniumalkyl-hydroxy)-1,3-(diaza)-2- |
| | (oxo)-phenthiazin-1-yl |
| 2′-amino-2′-deoxyribose | 7-(guanidiniumalkylhydroxy)-1,3-(diaza)-2- |
| | (oxo)-phenoxazin-1-yl |
| 2′-alpha-Trifluoromethylcytidine | 7-(methyl)guanine |
| 2′-Azido-2′-deoxycytosine | 7-(propynyl)isocarbostyrilyl |
| 2′-azido-2′-deoxyribose | 7-(propynyl)isocarbostyrilyl |
| 2′-Azido-deoxyuridine | 7-propynyl(aza)indolyl |
| 2′-beta-Ethynyladenosine | 7-aminomethyl-7-deazaguanosine |
| 2′-beta-Ethynylguanosine | 7-cyano-7-deazaguanosine |
| 2′-beta-Ethynyluridine | 7-deaza-2-aminopurine |
| 2′-beta-Trifluoromethyluridine | 7-deaza-2,6-diaminopurine |
| 2′-beta-Ethynylcytidine | 7-deaza-2-amino-purine |
| 2′-bromo-deoxyuridine | 7-deaza-8-aza-2,6-diaminopurine |
| 2′-deoxyuridine | 7-deaza-8-aza-2-aminopurine |
| 2′-Deoxy-2′,2′-difluoroadenosine | 7-deaza-8-aza-adenine |
| 2′-Deoxy-2′,2′-difluorocytidine | 7-deaza-8-aza-adenosine |
| 2′-Deoxy-2′,2′-difluoroguanosine | 7-deaza-8-aza-guanosine |
| 2′-Deoxy-2′,2′-difluorouridine | 7-deaza-adenosine |
| 2′-Deoxy-2′-alpha-aminocytidine | 7-deaza-guanosine |
| 2′-Deoxy-2′-alpha-aminouridine TP | 7-deaza-inosinyl |
| 2′-Deoxy-2′-alpha-azidocytidine | 7-methyl-8-oxo-guanosine |
| 2′-Deoxy-2′-alpha-azidouridine TP | 7-methyladenine |
| 2′-Deoxy-2′-alpha-mercaptoadenosine | 7-methylguanosine |
| 2′-Deoxy-2′-alpha-mercaptocytidine | 7-methylinosine |
| 2′-Deoxy-2′-alpha-mercaptoguanosine | 7-substituted 1-(aza)-2-(thio)-3-(aza)- |
| | phenoxazin-1-yl |
| 2′-Deoxy-2′-alpha-thiomethoxyadenosine | 7-substituted 1,3-(diaza)-2-(oxo)-phenoxazin- |
| | 1-yl |
| 2′-Deoxy-2′-alpha-thiomethoxycytidine | 8-(alkenyl)adenine |
| 2′-Deoxy-2′-alpha-thiomethoxyguanosine | 8-(alkenyl)guanine |
| 2′-Deoxy-2′-alpha-thiomethoxyuridine | 8-(alkyl)adenine |
| 2′-Deoxy-2′-alpha-mercaptouridine | 8-(alkyl)guanine |
| 2′-Deoxy-2′-beta-aminoadenosine | 8-(alkynyl)adenine |
| 2′-Deoxy-2′-beta-aminoguanosine | 8-(alkynyl)guanine |
| 2′-Deoxy-2′-beta-aminouridine | 8-(amino)adenine |
| 2′-Deoxy-2′-beta-azidoadenosine | 8-(amino)guanine |
| 2′-Deoxy-2′-beta-azidocytidine | 8-(halo)adenine |
| 2′-Deoxy-2′-beta-azidoguanosine | 8-(halo)guanine |
| 2′-Deoxy-2′-beta-azidouridine | 8-(hydroxyl)adenine |
| 2′-Deoxy-2′-beta-aminocytidine | 8-(hydroxyl)guanine |
| 2′-Deoxy-2′-beta-bromoadenosine | 8-(thioalkyl)adenine |
| 2′-Deoxy-2′-beta-bromocytidine | 8-(thioalkyl)guanine |
| 2′-Deoxy-2′-beta-bromoguanosine | 8-(thiol)adenine |
| 2′-Deoxy-2′-beta-bromouridine | 8-(thiol)guanine |
| 2′-Deoxy-2′-beta-chloroadenosine | 8-Aza-adenosine |
| 2′-Deoxy-2′-beta-chlorocytidine | 8-azido-adenosine |
| 2′-Deoxy-2′-beta-chloroguanosine | 8-bromo-adenosine |
| 2′-Deoxy-2′-beta-chlorouridine | 8-bromo-guanosine |
| 2′-Deoxy-2′-beta-fluoroadenosine | 8-oxo-guanosine |
| 2′-Deoxy-2′-beta-fluorocytidine | 8-Trifluoromethyladenosine |
| 2′-Deoxy-2′-beta-fluoroguanosine | 9-(methyl)-imidizopyridinyl |
| 2′-Deoxy-2′-beta-fluorouridine | 9-Deazaadenosine |
| 2′-Deoxy-2′-beta-iodoadenosine | 9-Deazaguanosine |
| 2′-Deoxy-2′-beta-iodocytidine | alkene containing backbones |
| 2′-Deoxy-2′-beta-iodoguanosine | alkyl phosphonates |
| 2′-Deoxy-2′-beta-iodouridine | allyamino-thymidine |
| 2′-Deoxy-2′-beta-mercaptoadenosine | allyamino-uracil |
| 2′-Deoxy-2′-beta-mercaptocytidine | alpha-thio-cytidine |
| 2′-Deoxy-2′-beta-mercaptoguanosine | alpha-thio-guanosine |
| 2′-Deoxy-2′-beta-mercaptouridine | alpha-thio-pseudo-uridine |
| 2′-Deoxy-2′-beta-thiomethoxyadenosine | alpha-thio-uridine |
| 2′-Deoxy-2′-beta-thiomethoxycytidine TP | altriol |
| 2′-Deoxy-2′-beta-thiomethoxyuridine | aminoalkylphosphoramidates |
| 2′-deoxyuridine | aminoalkylphosphotriesters |
| 2′-F-5-Methyl-2′-deoxyuridine | aminoindolyl |
| 2′-Fluoro | anthracenyl |
| 2′-fluoro-modified bases | archaeosine |
| 2′-fluorouridine | aza cytosine |
| 2′-methyl, 2′-amino, 2′-azido, 2′-fluoro- | aza thymidine |
| adenine | |
| 2′-methyl, 2′-amino, 2′-azido, 2′-fluroo- | aza uracil |
| cytidine | |
| 2′-OH-ara-adenosine | aza adenine |
| 2′-OH-ara-cytidine | azaguanine |
| 2′-OH-ara-guanosine | bis-ortho-(aminoalkylhydroxy)-6-phenyl- |
| | pyrrolo-nvrimidin-2-on-3-yl |
| 2′-OH-ara-uridine | bis-ortho-substituted-6-phenyl-pyrrolo- |
| | pyrimidin-2-on-3-yl |
| 2′-OMe-2-Aminoadenosine | boranophosphates |
| 2′-OMe-5-Me-uridine | —CH2—O—N(CH3)—CH2— |
| 2′-OMe-pseudouridine | —CH2—N(CH3)—N(CH3)—CH2— |
| 2′-O-Methyl-5-(1-propynyl)cytidine | —CH2—NH—CH2— |
| 2′-O-Methyl-5-(1-propynyl)uridine | chiral phosphonates |
| 2′-O-methyladenosine | chiral phosphorothioates |
| 2′-O-methylation | Constrained nucleic acid (CNA) |
| 2′-O-methylcytidine | deaza cytosine |
| 2′-O-methylguanosine | deaza guanine |
| 2′-O-methylinosine | deaza thymidine |
| 2′-O-methyl-ribose | deaza uracil |
| 2′-O-methyluridine | deazaadenine |
| 2′-O-ribosyladenosine (phosphate) | deoxy-thymidine |
| 2-(alkyl)guanine | difluorotolyl |
| 2-(alkyl)adenine | dihydropseudouridine |
| 2-(amino)adenine | dihydrouridine |
| 2-(aminoalkyl)adenine | DNA |
| 2-(aminopropyl)adenine | epoxyqueuosine |
| 2-(halo)adenine | Fluoro hexitol nucleic acid (FHNA) |
| 2-(methylthio) N6 (isopentenyl)adenine | formacetyl and thioformacetyl backbones |
| 2-(propyl)adenine | Formycin A |
| 2-(propyl)guanine | Formycin B |
| 2-(thio)cytosine | galactosyl-queuosine |
| 2-(thio)uracil | GNA (glycol nucleic acid) |
| 2,2′-anhydro-cytidine | hydroxywybutosine |
| 2,2′-anhydro-uridine | hypoxanthine |
| 2,4-(dithio)pseudouracil | imidizopyridinyl |
| 2,4,5-(trimethyl)phenyl | inosinyl |
| 2,6-(diamino)purine | isocarbostyrilyl |
| 2,6-diaminopurine | isoguanosine |
| 2′-alpha-ethynyladenosine | isopentenyladenosine |
| 2′-Amino-2′-deoxy-guanosine | isowyosme |
| 2′-Amino-2′-deoxy-uridine | 1-Alkyl-6-homoallyl-pseudo-uridine |
| 2-amino-6-Chloro-purine | 1-Methyl-6-(2,2,2-Trifluoroethyl)pseudo- |
| | uridine |
| 2-aminoadenine | 1-Methyl-6-(4-thiomorpholino)-pseudo- |
| | uridine |
| 2-Aminoadenosine | 1-Methyl-6-azido-pseudo-uridine |
| 2-aminopurine | 1-Methyl-6-chloro-pseudo-uridine |
| 2-Amino-riboside | 1-Methyl-6-dimethylamino-pseudo-uridine |
| 2-aza-inosinyl | 1-Methyl-6-ethoxy-pseudo-uridine |
| 2′-azido-2′-deoxyadenosine | 1-Methyl-6-ethylcarboxylate-pseudo-uridine |
| 2′-Azido-2′-deoxy-guanosine | 1-Methyl-6-fluoro-pseudo-uridine |
| 2′-Azido-2′-deoxy-uridine | 1-Methyl-6-hydroxy-pseudo-uridine |
| 2-Azidoadenosine | 1-Methyl-6-iodo-pseudo-uridine |
| 2′-beta-Trifluoromethyladenosine | 1-Methyl-6-methylamino-pseudo-uridine |
| 2′-beta-Trifluoromethylguanosine | 1-[3-(2-{2-[2-(2-Aminoethoxy)-ethoxy]- |
| | ethoxy}-ethoxy)-propionyl]pseudouridine |
| 2-Bromoadenosine | 1-Alkyl-6-(1-propynyl)-pseudo-uridine |
| 2′-beta-Trifluoromethylcytidine | 1-Alkyl-6-(2-propynyl)-pseudo-uridine |
| 2-Chloroadenosine | 1-Alkyl-6-allyl-pseudo-uridine |
| 2′-Deoxy-2′-alpha-aminoadenosine | 1-Alkyl-6-ethynyl-pseudo-uridine |
| 2′-Deoxy-2′-alpha-aminoguanosine | 1-Alkyl-6-vinyl-pseudo-uridine |
| 2′-Deoxy-2′-alpha-azidoadenosine | 1-Biotinyl-PEG2-pseudouridine |
| 2′-Deoxy-2′-alpha-azidoguanosine | 1-methyl-1-deaza-pseudoisocytidine |
| 2′-Deoxy-2′-beta-thiomethoxyguanosine | 1-methyl-1-deaza-pseudouridine |
| 2′-Fluor-N4-Bz-cytidine | 1-Methyl-3-(3-amino-3- |
| | carboxyproovl)pseudo-Uridine |
| 2′-fluoro-2′-deoxyribose | 1-Methyl-3-(3-amino-3-carboxypropyl) |
| | pseudouridine |
| 2-Fluoroadenosine | 1-methyl-3-(3-amino-5- |
| | carboxypropyl)pseudouridine |
| 2′-Fluoro-N2-isobutyl-guanosine | 1-Methyl-6-(4-morpholino)-pseudo-uridine |
| 2′-Fluoro-N4-Acetyl-cytidine | 1-Methyl-6-(substituted phenyl)pseudo- |
| | uridine |
| 2′-Fluoro-N6-Bz-deoxyadenosine | 1-Methyl-6-butyl-pseudo-uridine |
| 2-Iodoadenosine | 1-Methyl-6-ethyl-pseudo-uridine |
| 2-Mercaptoadenosine | 1-Methyl-6-formyl-pseudo-uridine |
| 2-methoxy-4-thio-pseudouridine | 1-Methyl-6-iso-propyl-pseudo-uridine |
| 2-methoxy-4-thio-uridine | 1-Methyl-6-methoxy-pseudo-uridine |
| 2-methoxy-5-methyl-cytidine | 1-Methyl-6-phenyl-pseudo-uridine |
| 2-methoxy-adenine | 1-Methyl-6-propyl-pseudo-uridine |
| 2-methoxy-cytidine | 1-Methyl-6-tert-butyl-pseudo-uridine |
| 2-methoxyuridine | 1-methyl-6-thio-guanosine |
| 2′-methyl, 2′-amino, 2′-azido, 2′-fluro- | 1-Methyl-6-trifluoromethyl-pseudo-uridine |
| guanosine | |
| 2′-methyl, 2′-amino, 2′-azido, 2′fluro-uridine | Locked nucleic acid (LNA) |
| 2-methyladenosine | 1-taurinomethyl-1-methyl-uridine |
| 2-methylpseudouridine | 1-taurinomethyl-4-thio-uridine |
| 2-methylthioadenine | lysidine |
| 2-methylthio-N6 isopentenyladenosine | mannosyl-queuosine |
| 2-methylthio-N6-(cis- | Methyl phosphonate |
| hydroxyisopentenyl)adenosine | |
| 2-methylthio-N6-hydroxynorvalyl | methylene (methylimino) |
| carbamoyladenosine | |
| 2-methylthio-N6-isopentenyladenosine | methylene formacetyl and thioformacetyl |
| | backbones |
| 2-methylthio-N6-methyladenosine | methyleneimino and methylenehydrazino |
| | backbones |
| 2-methylthio-N6-threonyl | methylphosphonates |
| carbamoyladenosine | |
| 2′-O-methoxyethyl (MOE) | methylwyosine |
| 2′-O-methoxyethylribose (MOE) | morpholino linkages |
| 2′-O-methyl | mosme |
| 2′-O-methyladenosine | N (methyl)guanine |
| 2′-O-methylcytidine | —N(CH3)—CH2—CH2— |
| 2′-O-methylguanosine | N-(methyl)guanine |
| 2′-O-methylinosine | N2,7,2′-O-trimethylguanosine |
| 2′O-methyl-N2-isobutyl-guanosine | N2,2′-O-dimethylguanosine |
| 2′-O-Methyl-N4-Acetyl-cytidine | N2,7-dimethylguanosine |
| 2′-O-methyl-N4-Bz-cytidine | N2,N2,2′-O-trimethylguanosine |
| 2′-O-methyl-N6-Bz-deoxyadenosine | N2,N2,7-trimethylguanosine |
| 2′-O-methylpseudouridine | N2,N2-dimethyl-6-thio-guanosine |
| 2′-O-methyluridine | N2,N2-dimethylguanosine |
| 2′-O-ribosyladenosine (phosphate) | N2-isobutyl-guanosine |
| 2′-O-ribosylguanosine (phosphate) | N2-methyl-6-thio-guanosine |
| 2-oxo-7-aminopyridopyrimidin-3-yl | N2-methylguanosine |
| 2-oxo-pyridopyrimidine-3-yl | N2-substituted purines |
| 2-pyridinone | N3 (methyl)uracil |
| 2-thio-1-methyl-1-deaza-pseudouridine | N4 (acetyl)cytosine |
| 2-thio-1-methyl-pseudouridine | N4,2′-O-dimethylcytidine |
| 2-thio-2′-O-methyluridine | N4,N4-Dimethyl-2′-OMe-Cytidine |
| 2-thio-5-aza-uridine | N4-acetyl-2′-O-methylcytidine |
| 2-thio-5-methyl-cytidine | N4-acetylcytidine |
| 2-thiocytidine | N4-Amino-cytidine |
| 2-thio-dihydropseudouridine | N4-Benzoyl-cytidine |
| 2-thio-dihydrouridine | N4-methylcytidine |
| 2-thio-pseudouridine | N6-(19-Amino-pentaoxanonadecyl)adenosine |
| 2-thiouridine | N6-(cis-hydroxyisopentenyl)adenosine |
| 2-thio-zebularine | N6-(isopentyl)adenine |
| 2-Trifluoromethyladenosine | N6-(methyl)adenine |
| 3-(deaza)-5-(aza)cytosine | N6,N6 (dimethyl)adenine |
| 3-(methyl)cytosine | N6,2′-O-dimethyladenosine |
| 3-nitropyrrole | N6,N6,2′-O-trimethyladenosine |
| 3-(3-amino-3-carboxypropyl)uracil | N6,N6-dimethyladenosine |
| 3-(3-amino-3-carboxypropyl)uridine | N6-acetyladenosine |
| 3-(alkyl)cytosine | N6-cis-hydroxy-isopentenyl-adenosine |
| 3-(methyl)-7-(propynyl)isocarbostyrilyl | N6-glycinylcarbamoyladenosine |
| 3-(methyl)cytidine | N6-hydroxynorvalylcarbamoyladenosine |
| 3-(methyl)isocarbostyrilyl | N6-isopentenyladenosine |
| 3,2′-O-dimethyluridine | N6-methyl-2-amino-purine |
| 3′-alkylene phosphonates | N6-methyladenosine |
| 3-alkyl-pseudouridine | N6-methyl-N6-threonylcarbamoyladenosine |
| 3′-aminophosphoramidate | N6-substituted purines |
| 3-deaza-3-bromoadenosine | N6-threonylcarbamoyladenosine |
| 3-deaza-3-chloroadenosine | N-alkylated derivative |
| 3-deaza-3-fluoroadenosine | napthalenyl |
| 3-deaza-3-iodoadenosine | nitrobenzimidazolyl |
| 3-deazaadenosine | nitroimidazolyl |
| 3′-ethynylcytidine | nitroindazolyl |
| 3-methylcytidine | nitropyrazolyl |
| 3-methyl-pseudouridine | N1-methyl-adenosine |
| 3-methyluridine | N1-methyl-guanosine |
| 4′-azidoadenosine | nubularine |
| 4′-azidouridine | 06-substituted purines |
| 4′-ethynyladenosine | O-alkylated derivative |
| 4′-ethynylcytidine | oligonucleosides with heteroatom |
| | internucleoside linkage |
| 4′-ethynylguanosine | ortho-(aminoalkylhydroxy)-6-phenyl-pyrrolo- |
| | pyrimidin-2-on-3-yl |
| 4′-ethynyluridine | ortho-substituted-6-phenyl-pyrrolo-pyrimidin- |
| | 2-on-3-yl |
| 4-(fluoro)-6-(methyl)benzimidazole | Oxoformycin TP |
| 4-(methyl)benzimidazole | para-(aminoalkylhydroxy)-6-phenyl-pyrrolo- |
| | pyrimidin-2-on-3-yl |
| 4-(methyl)indolyl | para-substituted-6-phenyl-pyrrolo-pyrimidin- |
| | 2-on-3-yl |
| 4-(thio)pseudouracil | pentacenyl |
| 4-(thio)uracil | peroxywybutosine |
| 4,2′-O-dimethylcytidine | phenanthracenyl |
| 4,6-(dimethyl)indolyl | phenyl |
| 4′-azidocytidine | phosphinates |
| 4′-azidoguanosine | phosphonoacetates |
| 4′-carbocyclic adenosine | phosphoramidates |
| 4′-carbocyclic cytidine | Phosphorodiamidate Morpholino Oligomer |
| | (PMO) |
| 4′-carbocyclic guanosine | phosphorodithioates |
| 4′-carbocyclic uridine | Phosphorothioate |
| 4-demethylwyosine | phosphorothioate internucleoside linkages |
| 4-methoxy-1-methyl-pseudoisocytidine | phosphorothioates |
| 4-methoxy-2-thio-pseudouridine | phosphotriesters |
| 4-methoxy-pseudoisocytidine | PNA |
| 4-methoxy-pseudouridine | propynyl-7-(aza)indolyl |
| 4-methylcytidine | pseudoisocytidine |
| 4-thio-1-methyl-1-deaza-pseudoisocytidine | Pseudo-iso-cytidine |
| 4-thio-1-methyl-pseudoisocytidine | pseudouracil |
| 4-thio-1-methyl-pseudouridine | pseudouridine |
| 4-thio-pseudoisocytidine | Pseudouridine 1-(4-methylbenzenesulfonic |
| | acid) |
| 4-thio-pseudouridine | Pseudouridine 1-(4-methylbenzoic acid) TP |
| 4-thiouracil | Pseudouridine 1-methylphosphonic acid |
| 4-thiouridine | Pseudouridine 1-[3-(2-ethoxy)]propionic acid |
| 5 (halo)cytosine | Pseudouridine 1-[3-{2-(2-[2-(2-ethoxy)- |
| | ethoxy]-ethoxy)-ethoxy}]propionic acid |
| 5 (methyl) 4 (thio)uracil | Pseudouridine 1-[3-{2-(2-[2-{2(2-ethoxy)- |
| | ethoxy}-ethoxy]-ethoxy)-ethoxy}]propionic |
| | acid |
| 5 (methyl)cytosine | Pseudouridine 1-[3-{2-(2-[2-ethoxy]-ethoxy)- |
| | ethoxv}]propionic acid |
| 5 (methylaminomethyl)-2 (thio)uracil | Pseudouridine 1-[3-{2-(2-ethoxy)-ethoxv}] |
| | propionic acid |
| 5 (methylaminomethyl)-2,4 (dithio)uracil | Pseudouridine TP 1-methylphosphonic acid |
| | diethyl ester |
| 5 (methylaminomethyl)-4 (thio)uracil | Pseudo-uridine-1-2-ethanoic acid |
| 5 (propynyl)cytosine | Pseudo-uridine-N1-5-pentanoic acid |
| 5 (propynyl)uracil | Pseudo-uridine-N1-3-propionic acid |
| 5 (trifluoromethyl)cytosine | Pseudo-uridine-N1-4-butanoic acid |
| 5 (trifluoromethyl)uracil | Pseudo-uridine-N1-6-hexanoic acid |
| 5 nitroindole | Pseudo-uridine-N1-methyl-p-benzoic acid |
| 5 substituted pyrimidines | Pseudo-uridine-N1-p-benzoic acid |
| 5-(1,3-diazole-1-alkyl)uracil | Pseudo-uridine-N1-7-heptanoic acid |
| 5-(1-Propynyl)ara-cytidine | pyrenyl |
| 5-(1-Propynyl)ara-uridine | pyridin-4-one ribonucleoside |
| 5-(2-aminopropyl)uracil | pyridopyrimidin-3-yl |
| 5-(2-carbomethoxyvinyl)uridine | pyridopyrimidin-3-yl, 2-oxo-7-amino- |
| | pyridopyrimidin-3-yl |
| 5-(2-Chloro-phenyl)-2-thiocytidine | pyrrolo-cytidine |
| 5-(2-Furanyl)uridine | pyrrolo-pseudoisocytidine |
| 5-(4-Amino-phenyl)-2-thiocytidine | pyrrolo-pyrimidin-2-on-3-yl |
| 5-(alkyl)-2-(thio)pseudouracil | pyrrolopyrimidinyl |
| 5-(alkyl)-4 (thio)pseudouracil | pyrrolopyrizinyl |
| 5-(alkyl)-2,4 (dithio)pseudouracil | Pyrrolosine |
| 5-(alkyl)cytosine | siloxane backbones |
| 5-(alkyl)pseudouracil | stilbenzyl |
| 5-(alkyl)uracil | substituted 1,2,4-triazoles |
| 5-(alkynyl)cytosine | sulfamate backbones |
| 5-(alkynyl)uracil | sulfide sulfoxide and sulfone backbones |
| 5-(allylamino)uracil | sulfonate and sulfonamide backbones |
| 5-(aminoalkyl)uracil | tetracenyl |
| 5-(carboxyhydroxymethyl)uridine | thio-adenosine |
| 5-(carboxyhydroxymethyl)uridine methyl | thionoalkylphosphonates |
| ester | |
| 5-(cyanoalkyl)uracil | thionoalkylphosphotriesters |
| 5-(dialkylaminoalkyl)uracil | thionophosphoramidates |
| 5-(dimethylaminoalkyl)uracil | Tricyclo-DNA (tcDNA) |
| 5-(guanidiniumalkyl)uracil | tubercidine |
| 5-(halo)cytosine | undermodified hydroxywybutosine |
| 5-(halo)uracil | uridine 5-oxyacetic acid |
| 5-(iso-Pentenylaminomethyl)-2-thiouridine | uridine 5-oxyacetic acid methyl ester |
| 5-(iso-Pentenylaminomethyl)-2′-O- | wybutosine |
| methyluridine | |
| 5-(iso-Pentenylaminomethyl)uridine | wyosme |
| 5-(1,3-diazole-1-alkyl)uracil | xanthine |
| 5-(methoxy)uracil | Xanthosine |
| 5-(methoxycarbonylmethyl)-2-(thio)uracil | zebularine |

A TREM may comprise a non-naturally occurring modification in each of the [ASt Domain1], the [DH Domain], the [ACH Domain], the [VL Domain], the [TH Domain], and the [ASt Domain2]. In some embodiments, the TREM comprises a nucleotide sugar modification in each of the [ASt Domain1], the [DH Domain], the [ACH Domain], the [VL Domain], the [TH Domain], and the [ASt Domain2]. In some embodiments, the TREM comprises an internucleotide modification in each of the [DH Domain] and the [ACH Domain]. In some embodiments, the TREM comprises twenty-one non-naturally occurring modifications. In some embodiments, the TREM comprises fifteen 2′-O-methyl modifications. In some embodiments, the TREM comprises one 2′-fluoro modification. In some embodiments, the TREM comprises five phosphorothioate modifications. In some embodiments, the TREM comprises fifteen 2′-O-methyl modifications in the [ASt Domain1], the [DH Domain], the [ACH Domain], the [VL Domain], the [TH Domain], and the [ASt Domain2], one 2′-fluoro modification in the [ACH Domain], and five phosphorothioate modifications in the [DH Domain] and the [ACH Domain]. In some embodiments, the TREM comprises the non-naturally occurring modification pattern of Pattern No: 1 in Table 6: 1-m, 14-m, 15-m, 16-m, 17-m, 18-m, 19-*, 20-m, 21-*, 33-f, 34-m, 35-*, 37-*, 38-*, 41-m, 42-m, 43-m, 44-m, 50-m, 52-m, 73-m.

A TREM may comprise a non-naturally occurring modification in each of the [ASt Domain1] and the [ASt Domain2]. In some embodiments, the TREM comprises a nucleotide sugar modification and an internucleotide modification in each of the [ASt Domain1] and the [ASt Domain2]. In some embodiments, the TREM comprises six non-naturally occurring modifications. In some embodiments, the TREM comprises three 2′-O-methyl modifications. In some embodiments, the TREM comprises three phosphorothioate modifications. In some embodiments, the TREM comprises three 2′-O-methyl modifications in the [ASt Domain1] and the [ASt Domain2] and three phosphorothioate modifications in the [ASt Domain1] and the [ASt Domain2]. In some embodiments, the TREM comprises the non-naturally occurring modification pattern of Pattern No: 2 in Table 6: 1-m*, 2-m*, 74-*, 75-m.

A TREM may comprise a non-naturally occurring modification in each of the [ASt Domain1], the [TH Domain], and the [ASt Domain2]. In some embodiments, the TREM comprises a nucleotide sugar modification in each of the [ASt Domain1] and the [ASt Domain2]. In some embodiments, the TREM comprises an internucleotide modification in each of the [ASt Domain1], the [TH Domain], and the [ASt Domain2]. In some embodiments, the TREM comprises seven non-naturally occurring modifications. In some embodiments, the TREM comprises three 2′-O-methyl modifications. In some embodiments, the TREM comprises four phosphorothioate modifications. In some embodiments, the TREM comprises three 2′-O-methyl modifications in the [ASt Domain1] and the [ASt Domain2] and four phosphorothioate modifications in the [ASt Domain1], the [TH Domain], and the [ASt Domain2]. In some embodiments, the TREM comprises the non-naturally occurring modification pattern of Pattern No: 3 in Table 6: 1-m*, 2-m*, 52-*, 74-*, 75-m.

A TREM may comprise a non-naturally occurring modification in each of the [ASt Domain1], the [DH domain], the [ACH Domain], the [VL Domain], the [TH Domain], and the [ASt Domain2]. In some embodiments, the TREM comprises a nucleotide sugar modification in each of the [ASt Domain1], the [DH Domain], the [ACH Domain], the [VL Domain], the [TH Domain], and the [ASt Domain2]. In some embodiments, the TREM comprises an internucleotide modification in each of the [DH Domain], the [ACH Domain], and the [TH Domain]. In some embodiments, the TREM comprises thirty-one non-naturally occurring modifications. In some embodiments, the TREM comprises twenty-three 2′-O-methyl modifications. In some embodiments, the TREM comprises eight phosphorothioate modifications. In some embodiments, the TREM comprises twenty-three 2′-O-methyl modifications in the [ASt Domain1], the [DH Domain], the [ACH Domain], the [VL Domain], the [TH Domain], and the [ASt Domain2] and eight phosphorothioate modifications in the [DH Domain], the [ACH Domain], and the [TH Domain]. In some embodiments, the TREM comprises the non-naturally occurring modification pattern of Pattern No: 4 in Table 6: 1-m, 2-m, 3-m, 4-m, 5-m, 6-m, 13-m, 14-m, 15-m, 16-m, 17-m, 18-m, 19-*, 20-m, 21-*, 25-m, 33-*, 34-m, 35-*, 37-*, 38-*, 49-m, 50-m, 51-m, 52-m, 53-m, 54-*, 55-*, 61-m, 65-m, 73-m.

A TREM may comprise a non-naturally occurring modification in each of the [ASt Domain1], the [DH Domain], the [TH Domain], and the [ASt Domain2]. In some embodiments, the TREM comprises a nucleotide sugar modification in each of the [ASt Domain1], the [DH Domain], the [TH Domain], and the [ASt Domain2]. In some embodiments, the TREM comprises an internucleotide modification in each of the [ASt Domain1] and the [ASt Domain2]. In some embodiments, the TREM comprises ten non-naturally occurring modifications. In some embodiments, the TREM comprises four 2′-O-methyl modifications. In some embodiments, the TREM comprises three 2′-fluoro modifications. In some embodiments, the TREM comprises three phosphorothioate modifications. In some embodiments, the TREM comprises four 2′-O-methyl modifications in the [ASt Domain1], the [TH Domain], and the [ASt Domain2], three 2′-fluoro modifications in the [DH Domain] and the [TH Domain], and three phosphorothioate modifications in the [ASt Domain1] and the [ASt Domain2]. In some embodiments, the TREM comprises the non-naturally occurring modification pattern of Pattern No: 5 in Table 6: 1-m*, 2-m*, 13-f, 14-f, 54-f, 59-m, 75-*, 76-m.

A TREM may comprise a non-naturally occurring modification in each of the [ASt Domain1], the [DH Domain], the [ACH Domain], the [VL Domain], the [TH Domain], and the [ASt Domain2]. In some embodiments, the TREM comprises a nucleotide sugar modification in each of the [ASt Domain1], the [DH Domain], the [ACH Domain], the [VL Domain], the [TH Domain] and the [ASt Domain2]. In some embodiments, the TREM comprises an internucleotide modification in each of the [ACH Domain] and the [TH Domain]. In some embodiments, the TREM comprises twenty-six non-naturally occurring modifications. In some embodiments, the TREM comprises sixteen 2′-O-methyl modifications. In some embodiments, the TREM comprises two 2′-fluoro modifications. In some embodiments, the TREM comprises eight phosphorothioate modifications. In some embodiments, the TREM comprises sixteen 2′-O-methyl modifications in the [ASt Domain1], the [DH Domain], the [ACH Domain], the [VL Domain], the [TH Domain], and the [ASt Domain2], two 2′-fluoro modifications in the [ACH Domain] and the [VL Domain], and eight phosphorothioate modifications in the [ACH Domain] and the [TH Domain]. In some embodiments, the TREM comprises the non-naturally occurring modification pattern of Pattern No: 6 in Table 6: 1-m, 13-m, 17-m, 18-m, 25-m, 33-*, 34-m, 35-*, 37-*, 38-*, 41-f, 42-m, 43-m, 44-f, 49-m, 50-m, 52-m, 54-*, 55-*, 56-m, 57-*, 58-*, 59-m, 61-m, 65-m, 73-m.

A TREM may comprise a non-naturally occurring modification in each of the [ASt Domain1], the [DH Domain], the [ACH Domain], the [VL Domain], the [TH Domain], and the [ASt Domain2]. In some embodiments, the TREM comprises a nucleotide sugar modification in each of the [ASt Domain1], the [DH Domain], the [ACH Domain], the [VL Domain], the [TH Domain], and the [ASt Domain2]. In some embodiments, the TREM comprises an internucleotide modification in each of the [DH Domain] and the [TH Domain]. In some embodiments, the TREM comprises twenty-five non-naturally occurring modifications. In some embodiments, the TREM comprises nineteen 2′-O-methyl modifications. In some embodiments, the TREM comprises two 2′-fluoro modifications. In some embodiments, the TREM comprises four phosphorothioate modifications. In some embodiments, the TREM comprises nineteen 2′-O-methyl modifications in the [ASt Domain1], the [DH Domain], the [ACH Domain], the [VL Domain], the [TH Domain], and the [ASt Domain2], two 2′-fluoro modifications in the [ACH Domain] and the [VL Domain], and four phosphorothioate modifications in the [DH Domain] and the [TH Domain]. In some embodiments, the TREM comprises the non-naturally occurring modification pattern of Pattern No: 7 in Table 6: 1-m, 13-m, 14-m, 15-m, 16-m, 17-m, 18-m, 19-*, 20-m, 21-*, 25-m, 41-f, 42-m, 43-m, 44-f, 49-m, 50-m, 51-m, 52-m, 53-m, 54-*, 55-*, 61-m, 65-m, 73-m.

A TREM may comprise a non-naturally occurring modification in each of the [ASt Domain1], the [DH Domain], the [ACH Domain], and the [ASt Domain2]. In some embodiments, the TREM comprises a nucleotide sugar modification in each of the [ASt Domain1], the [DH Domain], the [ACH Domain], and the [ASt Domain2]. In some embodiments, the TREM comprises ten non-naturally occurring modifications. In some embodiments, the TREM comprises eight 2′-O-methyl modifications. In some embodiments, the TREM comprises two 2′-fluoro modifications. In some embodiments, the TREM comprises eight 2′-O-methyl modifications in the [ASt Domain1], the [DH Domain], the [ACH Domain], and the [ASt Domain2] and two 2′-fluoro modifications in the [DH Domain] and the [ACH Domain]. In some embodiments, the TREM comprises the non-naturally occurring modification pattern of Pattern No: 8 in Table 6: 1-m, 19-m, 20-m, 21-f, 22-m, 40-f, 41-m, 42-m, 43-m, 73-m.

A TREM may comprise a non-naturally occurring modification in each of the [ASt Domain1], the [ACH Domain], and the [ASt Domain2]. In some embodiments, the TREM comprises a nucleotide sugar modification in each of the [ASt Domain1], the [ACH Domain], and the [ASt Domain2]. In some embodiments, the TREM comprises an internucleotide modification in the [ASt Domain1]. In some embodiments, the TREM comprises sixteen non-naturally occurring modifications. In some embodiments, the TREM comprises thirteen 2′-O-methyl modifications. In some embodiments, the TREM comprises two 2′-fluoro modifications. In some embodiments, the TREM comprises one phosphorothioate modification. In some embodiments, the TREM comprises thirteen 2′-O-methyl modifications in the [ASt Domain1], the [ACH Domain], and the [ASt Domain2], two 2′-fluoro modifications in the [ACH Domain], and one phosphorothioate modification in the [ASt Domain1]. In some embodiments, the TREM comprises the non-naturally occurring modification pattern of Pattern No: 9 in Table 6: 1-m*, 2-m, 3-m, 4-m, 5-m, 6-m, 27-m, 28-m, 29-f, 30-m, 40-f, 41-m, 42-m, 43-m, 73-m.

A TREM may comprise a non-naturally occurring modification in each of the [ASt Domain1], the [VL Domain], the [TH Domain], and the [ASt Domain2]. In some embodiments, the TREM comprises a nucleotide sugar modification in each of the [ASt Domain1], the [VL Domain], the [TH Domain], and the [ASt Domain2]. In some embodiments, the TREM comprises an internucleotide modification in the [ASt Domain1]. In some embodiments, the TREM comprises twenty-three non-naturally occurring modifications. In some embodiments, the TREM comprises twenty-one 2′-O-methyl modifications. In some embodiments, the TREM comprises one 2′-fluoro modification. In some embodiments, the TREM comprises one phosphorothioate modification. In some embodiments, the TREM comprises twenty-one 2′-O-methyl modifications in the [ASt Domain1], the [VL Domain], the [TH Domain], and the [ASt Domain2], one 2′-fluoro modification in the [TH Domain], and one phosphorothioate modification in the [ASt Domain1]. In some embodiments, the TREM comprises the non-naturally occurring modification pattern of Pattern No: 10 in Table 6: 1-m*, 2-m, 3-m, 4-m, 5-m, 6-m, 49-m, 50-m, 51-m, 52-m, 53-m, 61-m, 62-m, 63-f, 64-m, 65-m, 66-m, 67-m, 68-m, 69-m, 71-m, 72-m.

A TREM may comprise a non-naturally occurring modification in each of the [ASt Domain1], the [DH Domain], the [VL Domain], the [TH Domain], and the [ASt Domain2]. In some embodiments, the TREM comprises a nucleotide sugar modification in each of the [ASt Domain1], the [DH Domain], the [VL Domain], the [TH Domain], and the [ASt Domain2]. In some embodiments, the TREM comprises an internucleotide modification in the [ASt Domain1]. In some embodiments, the TREM comprises thirty-one non-naturally occurring modifications. In some embodiments, the TREM comprises twenty-eight 2′-O-methyl modifications. In some embodiments, the TREM comprises one 2′-fluoro modification. In some embodiments, the TREM comprises two phosphorothioate modifications. In some embodiments, the TREM comprises twenty-eight 2′-O-methyl modifications in the [ASt Domain1], the [DH Domain], the [VL Domain], the [TH Domain], and the [ASt Domain2], one 2′-fluoro modification in the [TH Domain], and two phosphorothioate modifications in the [ASt Domain1]. In some embodiments, the TREM comprises the non-naturally occurring modification pattern of Pattern No: 11 in Table 6: 1-m*, 2-m*, 3-m, 4-m, 5-m, 6-m, 10-m, 11-m, 12-m, 13-m, 22-m, 23-m, 24-m, 25-m, 49-m, 50-m, 51-m, 52-m, 53-m, 54-m, 61-m, 62-m, 63-f, 64-m, 65-m, 66-m, 67-m, 68-m, 73-m.

A TREM may comprise a non-naturally occurring modification in each of the [ASt Domain1], the [DH Domain], the [ACH Domain], the [VL Domain], the [TH Domain], and the [ASt Domain2]. In some embodiments, the TREM comprises a nucleotide sugar modification in each of the [ASt Domain1], the [DH Domain], the [ACH Domain], the [VL Domain], the [TH Domain], and the [ASt Domain2]. In some embodiments, the TREM comprises an internucleotide modification in each of the [ASt Domain1] and the [ACH Domain]. In some embodiments, the TREM comprises thirty-two non-naturally occurring modifications. In some embodiments, the TREM comprises twenty-three 2′-O-methyl modifications. In some embodiments, the TREM comprises four 2′-fluoro modifications. In some embodiments, the TREM comprises five phosphorothioate modifications. In some embodiments, the TREM comprises twenty-three 2′-O-methyl modifications in the [ASt Domain1], the [DH Domain], the [ACH Domain], the [VL Domain], the [TH Domain], and the [ASt Domain2], four 2′-fluoro modifications in the [ACH Domain] and the [VL Domain], and five phosphorothioate modifications in the [ASt Domain1] and the [ACH Domain]. In some embodiments, the TREM comprises the non-naturally occurring modification pattern of Pattern No: 12 in Table 6: 1-m*, 2-m*, 3-m, 4-m, 5-m, 6-m, 16-m, 17-m, 18-m, 20-m, 25-m, 33-f, 35-*, 37-*, 38-*, 41-m, 44-m, 45-m, 46-f, 47-f, 48-f, 49-m, 50-m, 51-m, 52-m, 53-m, 54-m, 56-m, 59-m, 73-m.

A TREM may comprise a non-naturally occurring modification in each of the [ASt Domain1], the [DH Domain], the [ACH Domain], the [VL Domain], the [TH Domain], and the [ASt Domain2]. In some embodiments, the TREM comprises a nucleotide sugar modification in each of the [ASt Domain1], the [DH Domain], the [ACH Domain], the [VL Domain], the [TH Domain], and the [ASt Domain2]. In some embodiments, the TREM comprises an internucleotide modification in the [ACH Domain]. In some embodiments, the TREM comprises twenty-nine non-naturally occurring modifications. In some embodiments, the TREM comprises twenty-one 2′-O-methyl modifications. In some embodiments, the TREM comprises five 2′-fluoro modifications. In some embodiments, the TREM comprises three phosphorothioate modifications. In some embodiments, the TREM comprises twenty-one 2′-O-methyl modifications in the [ASt Domain1], the [DH Domain], the [VL Domain], the [TH Domain], and the [ASt Domain2], five 2′-fluoro modifications in the [ACH Domain], the [VL Domain], and the [TH Domain], and three phosphorothioate modifications in the [ACH Domain]. In some embodiments, the TREM comprises the non-naturally occurring modification pattern of Pattern No: 13 in Table 6: 1-m, 2-m, 3-m, 4-m, 5-m, 6-m, 16-m, 17-m, 18-m, 33-f, 35-*, 37-*, 38-*, 44-m, 45-m, 46-f, 47-f, 48-f, 56-m, 59-m, 61-m, 62-m, 63-f, 64-m, 65-m, 66-m, 67-m, 68-m, 73-m.

A TREM may comprise a non-naturally occurring modification in each of the [ASt Domain1], the [DH Domain], the [ACH domain], the [VL Domain], the [TH Domain], and the [ASt Domain2]. In some embodiments, the TREM comprises a nucleotide sugar modification in each of the [ASt Domain1], the [DH Domain], the [ACH domain], the [VL Domain], the [TH Domain], and the [ASt Domain2]. In some embodiments, the TREM comprises an internucleotide modification in each of the [ASt Domain1] and the [ACH Domain]. In some embodiments, the TREM comprises forty-three non-naturally occurring modifications. In some embodiments, the TREM comprises thirty-one 2′-O-methyl modifications. In some embodiments, the TREM comprises seven 2′-fluoro modifications. In some embodiments, the TREM comprises five phosphorothioate modifications. In some embodiments, the TREM comprises forty-three 2′-O-methyl modifications in the [ASt Domain1], the [DH Domain], the [VL Domain], the [TH Domain], and the [ASt Domain2], seven 2′-fluoro modifications in the [DH Domain], the [ACH Domain], the [VL Domain], and the [TH Domain], and five phosphorothioate modifications in the [ASt Domain1] and the [ACH Domain]. In some embodiments, the TREM comprises the non-naturally occurring modification pattern of Pattern No: 14 in Table 6: 1-m*, 2-m*, 3-m, 4-m, 5-m, 6-m, 10-m, 11-m, 12-m, 13-m, 14-m, 15-f, 16-m, 17-m, 18-m, 19-f, 20-m, 22-m, 23-m, 24-m, 25-m, 33-f, 35-*, 37-*, 38-*, 44-m, 45-m, 46-f, 47-f, 48-f, 56-m, 59-m, 61-m, 62-m, 63-f, 64-m, 65-m, 66-m, 67-m, 68-m, 73-m.

A TREM may comprise a non-naturally occurring modification in each of the [ASt Domain1], the [DH Domain], the [ACH Domain], the [VL Domain], the [TH Domain], and the [ASt Domain2]. In some embodiments, the TREM comprises a nucleotide sugar modification in each of the [ASt Domain1], the [DH Domain], the [ACH Domain], the [VL Domain], the [TH Domain], and the [ASt Domain2]. In some embodiments, the TREM comprises an internucleotide modification in each of the [ASt Domain1] and the [ACH Domain]. In some embodiments, the TREM comprises twenty-six non-naturally occurring modifications. In some embodiments, the TREM comprises seventeen 2′-O-methyl modifications. In some embodiments, the TREM comprises four 2′-fluoro modifications. In some embodiments, the TREM comprises five phosphorothioate modifications. In some embodiments, the TREM comprises seventeen 2′-O-methyl modifications in the [ASt Domain1], the [DH Domain], the [ACH Domain], the [VL Domain], the [TH Domain], and the [ASt Domain2], four 2′-fluoro modifications in the [ACH Domain] and the [VL Domain], and five phosphorothioate modifications in the [ASt Domain1] and the [ACH Domain]. In some embodiments, the TREM comprises the non-naturally occurring modification pattern of Pattern No: 15 in Table 6: 1-m*, 2-m*, 3-m, 4-m, 5-m, 6-m, 16-m, 17-m, 18-m, 20-m, 25-m, 33-f, 35-*, 37-*, 38-*, 41-m, 44-m, 45-m, 46-f, 47-f, 48-f, 56-m, 59-m, 73-m.

A TREM may comprise a non-naturally occurring modification in each of the [ASt Domain1], the [DH Domain], the [TH Domain], and the [ASt Domain2]. In some embodiments, the TREM comprises a nucleotide sugar modification in each of the [ASt Domain1], the [DH Domain], the [TH Domain], and the [ASt Domain2]. In some embodiments, the TREM comprises six non-naturally occurring modifications. In some embodiments, the TREM comprises six 2′-O-methyl modifications. In some embodiments, the TREM comprises six 2′-O-methyl modifications in the [ASt Domain1], the [DH Domain], the [TH Domain], and the [ASt Domain2]. In some embodiments, the TREM comprises the non-naturally occurring modification pattern of Pattern No: 16 in Table 6: 1-m, 17-m, 18-m, 50-m, 52-m, 73-m.

A TREM may comprise a non-naturally occurring modification in each of the [ASt Domain1], the [DH Domain], the [TH Domain], and the [ASt Domain2]. In some embodiments, the TREM comprises a nucleotide sugar modification in each of the [ASt Domain1], the [DH Domain], the [TH Domain], and the [ASt Domain2]. In some embodiments, the TREM comprises an internucleotide modification in the [TH Domain]. In some embodiments, the TREM comprises fifteen non-naturally occurring modifications. In some embodiments, the TREM comprises eleven 2′-O-methyl modifications. In some embodiments, the TREM comprises two 2′-fluoro modifications. In some embodiments, the TREM comprises two phosphorothioate modifications. In some embodiments, the TREM comprises eleven 2′-O-methyl modifications in the [ASt Domain1], the [DH Domain], the [TH Domain], and the [ASt Domain2], two 2′-fluoro modifications in the [DH Domain], and two phosphorothioate modifications in the [TH Domain]. In some embodiments, the TREM comprises the non-naturally occurring modification pattern of Pattern No: 17 in Table 6: 1-m, 14-m, 15-f, 16-m, 17-m, 18-m, 19-f, 20-m, 54-m*, 55-*, 56-m, 57-m, 59-m, 73-m.

A TREM may comprise a non-naturally occurring modification in each of the [ASt Domain1], the [DH Domain], the [ACH Domain], the [VL Domain], the [TH Domain], and the [ASt Domain2]. In some embodiments, the TREM comprises a nucleotide sugar modification in each of the [ASt Domain1], the [DH Domain], the [ACH Domain], the [VL Domain], the [TH Domain], and the [ASt Domain2]. In some embodiments, the TREM comprises an internucleotide modification in each of the [DH Domain], the [ACH Domain], the [VL Domain], and the [TH Domain]. In some embodiments, the TREM comprises twenty-nine non-naturally occurring modifications. In some embodiments, the TREM comprises fifteen 2′-O-methyl modifications. In some embodiments, the TREM comprises four 2′-fluoro modifications. In some embodiments, the TREM comprises ten phosphorothioate modifications. In some embodiments, the TREM comprises fifteen 2′-O-methyl modifications in the [ASt Domain1], the [DH Domain], the [ACH Domain], the [TH Domain], and the [ASt Domain2], four 2′-fluoro modifications in the [ACH Domain], the [VL Domain], and the [TH Domain], and ten phosphorothioate modifications in the [DH Domain], the [ACH Domain], the [VL Domain], and the [TH Domain]. In some embodiments, the TREM comprises the non-naturally occurring modification pattern of Pattern No: 18 in Table 6: 1-m, 10-m, 13-m, 17-*, 18-m, 19-*, 20-m, 25-m, 29-m, 33-f, 35-*, 37-*, 38-*, 41-f*, 43-m, 44-f, 46-*, 50-m, 52-m, 54-*, 55-*, 56-m, 57-*, 62-m, 63-f, 65-m, 71-m, 73-m.

A TREM may comprise a non-naturally occurring modification in each of the [ASt Domain1], the [TH Domain], and the [ASt Domain2]. In some embodiments, the TREM comprises a nucleotide sugar modification in each of the [ASt Domain1], the [TH Domain], and the [ASt Domain2]. In some embodiments, the TREM comprises thirteen non-naturally occurring modifications. In some embodiments, the TREM comprises twelve 2′-O-methyl modifications. In some embodiments, the TREM comprises one 2′-fluoro modification. In some embodiments, the TREM comprises twelve 2′-O-methyl modifications in the [ASt Domain1], the [TH Domain], and the [ASt Domain2] and one 2′-fluoro modification in the [ASt Domain2]. In some embodiments, the TREM comprises the non-naturally occurring modification pattern of Pattern No: 19 in Table 6: 1-m, 61-m, 62-m, 64-m, 65-m, 66-m, 67-m, 68-m, 69-m, 70-f, 71-m, 72-m, 73-m.

A TREM may comprise a non-naturally occurring modification in each of the [ASt Domain1], the [DH Domain], the [ACH Domain], the [VL Domain], the [TH Domain], and the [ASt Domain2]. In some embodiments, the TREM comprises a nucleotide sugar modification in each of the [ASt Domain1], the [DH Domain], the [ACH Domain], the [VL Domain], the [TH Domain], and the [ASt Domain2]. In some embodiments, the TREM comprises an internucleotide modification in the [ACH Domain]. In some embodiments, the TREM comprises nineteen non-naturally occurring modifications. In some embodiments, the TREM comprises thirteen 2′-O-methyl modifications. In some embodiments, the TREM comprises three 2′-fluoro modifications. In some embodiments, the TREM comprises three phosphorothioate modifications. In some embodiments, the TREM comprises thirteen 2′-O-methyl modifications in the [ASt Domain1], the [DH Domain], the [ACH Domain], the [VL Domain], the [TH Domain], and the [ASt Domain2], three 2′-fluoro modifications in the [ACH Domain], and three phosphorothioate modifications in the [ACH Domain]. In some embodiments, the TREM comprises the non-naturally occurring modification pattern of Pattern No: 20 in Table 6: 1-m, 14-m, 15-f, 16-m, 17-m, 18-m, 19-f, 20-m, 33-f, 35-*, 37-*, 38-*, 41-m, 42-m, 43-m, 44-m, 50-m, 52-m, 73-m.

A TREM may comprise a non-naturally occurring modification in each of the [ASt Domain1], the [DH Domain], the [ACH Domain], the [VL Domain], the [TH Domain], and the [ASt Domain2]. In some embodiments, the TREM comprises a nucleotide sugar modification in each of the [ASt Domain1], the [DH Domain], the [ACH Domain], the [VL Domain], the [TH Domain], and the [ASt Domain2]. In some embodiments, the TREM comprises an internucleotide modification in the [TH Domain]. In some embodiments, the TREM comprises twenty-eight non-naturally occurring modifications. In some embodiments, the TREM comprises twenty-six 2′-O-methyl modifications. In some embodiments, the TREM comprises one 2′-fluoro modification. In some embodiments, the TREM comprises one phosphorothioate modification. In some embodiments, the TREM comprises twenty-six 2′-O-methyl modifications in the [ASt Domain1], the [DH Domain], the [ACH Domain], the [VL Domain], the [TH Domain], and the [ASt Domain2], one 2′-fluoro modification in the [TH Domain], and one phosphorothioate modification in the [TH Domain]. In some embodiments, the TREM comprises the non-naturally occurring modification pattern of Pattern No: 21 in Table 6: 1-m, 2-m, 3-m, 4-m, 5-m, 6-m, 14-m, 15-m, 16-m, 17-m, 18-m, 20-m, 25-m, 41-m, 42-m, 43-m, 44-m, 49-m, 50-m, 52-m, 54-m, 55-*, 56-m, 57-f, 59-m, 61-m, 65-m, 73-m.

A TREM may comprise a non-naturally occurring modification in each of the [ASt Domain1], the [DH Domain], the [ACH Domain], the [VL Domain], the [TH Domain], and the [ASt Domain2]. In some embodiments, the TREM comprises a nucleotide sugar modification in each of the [ASt Domain1], the [DH Domain], the [ACH Domain], the [VL Domain], the [TH Domain], and the [ASt Domain2]. In some embodiments, the TREM comprises an internucleotide modification in each of the [ACH Domain] and the [TH Domain]. In some embodiments, the TREM comprises thirty-two non-naturally occurring modifications. In some embodiments, the TREM comprises twenty-five 2′-O-methyl modifications. In some embodiments, the TREM comprises three 2′-fluoro modifications. In some embodiments, the TREM comprises four phosphorothioate modifications. In some embodiments, the TREM comprises twenty-five 2′-O-methyl modifications in the [ASt Domain1], the [DH Domain], the [ACH Domain], the [VL Domain], the [TH Domain], and the [ASt Domain2], three 2′-fluoro modifications in the [ACH Domain], the [VL Domain], and the [TH Domain], and four phosphorothioate modifications in the [ACH Domain] and the [TH Domain]. In some embodiments, the TREM comprises the non-naturally occurring modification pattern of Pattern No: 22 in Table 6: 1-m, 2-m, 3-m, 4-m, 5-m, 6-m, 14-m, 15-m, 16-m, 17-m, 18-m, 20-m, 25-m, 33-f, 35-*, 37-*, 38-*, 41-m, 42-m, 43-m, 44-f, 49-m, 50-m, 52-m, 54-m, 55-*, 56-m, 57-f, 59-m, 61-m, 65-m, 73-m.

A TREM may comprise a non-naturally occurring modification in each of the [ASt Domain1], the [DH Domain], the [ACH Domain], the [VL Domain], the [TH Domain], and the [ASt Domain2]. In some embodiments, the TREM comprises a nucleotide sugar modification in each of the [ASt Domain1], the [DH Domain], the [ACH Domain], the [VL Domain], the [TH Domain], and the [ASt Domain2]. In some embodiments, the TREM comprises an internucleotide modification in each of the [ACH Domain] and the [TH Domain]. In some embodiments, the TREM comprises twenty-seven non-naturally occurring modifications. In some embodiments, the TREM comprises twenty-one 2′-O-methyl modifications. In some embodiments, the TREM comprises one 2′-fluoro modification. In some embodiments, the TREM comprises five phosphorothioate modifications. In some embodiments, the TREM comprises twenty-one 2′-O-methyl modifications in the [ASt Domain1], the [DH Domain], the [ACH Domain], the [VL Domain], the [TH Domain], and the [ASt Domain2], one 2′-fluoro modification in the [ACH Domain], and five phosphorothioate modifications in the [ACH Domain] and the [TH Domain]. In some embodiments, the TREM comprises the non-naturally occurring modification pattern of Pattern No: 23 in Table 6: 1-m, 2-m, 3-m, 4-m, 5-m, 6-m, 16-m, 17-m, 18-m, 20-m, 25-m, 33-f, 35-*, 37-*, 38-*, 41-m, 42-m, 43-m, 44-m, 49-m, 50-m, 52-m, 54-*, 55-*, 61-m, 65-m, 73-m.

A TREM may comprise a non-naturally occurring modification in each of the [ASt Domain1], the [DH Domain], the [ACH Domain], the [VL Domain], the [TH Domain], and the [ASt Domain2]. In some embodiments, the TREM comprises a nucleotide sugar modification in each of the [ASt Domain1], the [DH Domain], the [ACH Domain], the [VL Domain], the [TH Domain], and the [ASt Domain2]. In some embodiments, the TREM comprises an internucleotide modification in each of the [ACH Domain] and the [TH Domain]. In some embodiments, the TREM comprises thirty-four non-naturally occurring modifications. In some embodiments, the TREM comprises twenty-six 2′-O-methyl modifications. In some embodiments, the TREM comprises four 2′-fluoro modifications. In some embodiments, the TREM comprises four phosphorothioate modifications. In some embodiments, the TREM comprises twenty-six 2′-O-methyl modifications in the [ASt Domain1], the [DH Domain], the [ACH Domain], the [VL Domain], the [TH Domain], and the [ASt Domain2], four 2′-fluoro modifications in the [ACH Domain], the [VL Domain], and the [TH Domain], and four phosphorothioate modifications in the [ACH Domain] and the [TH Domain]. In some embodiments, the TREM comprises the non-naturally occurring modification pattern of Pattern No: 24 in Table 6: 1-m, 2-m, 3-m, 4-m, 5-m, 6-m, 10-m, 11-m, 12-m, 13-m, 16-m, 17-m, 18-m, 20-m, 25-m, 33-f, 35-*, 37-*, 38-*, 41-f, 42-m, 43-m, 44-f, 49-m, 50-m, 52-m, 54-m, 55-*, 56-in, 57-f, 59-m, 61-m, 65-m, 73-m.

A TREM may comprise a non-naturally occurring modification in each of the [ASt Domain1], the [DH Domain], the [ACH Domain], the [VL Domain], the [TH Domain], and the [ASt Domain2]. In some embodiments, the TREM comprises a nucleotide sugar modification in each of the [ASt Domain1], the [DH Domain], the [ACH Domain], the [VL Domain], the [TH Domain], and the [ASt Domain2]. In some embodiments, the TREM comprises an internucleotide modification in each of the [ACH Domain] and the [TH Domain]. In some embodiments, the TREM comprises thirty-seven non-naturally occurring modifications. In some embodiments, the TREM comprises twenty-nine 2′-O-methyl modifications. In some embodiments, the TREM comprises four 2′-fluoro modifications. In some embodiments, the TREM comprises four phosphorothioate modifications. In some embodiments, the TREM comprises twenty-nine 2′-O-methyl modifications in the [ASt Domain1], the [DH Domain], the [ACH Domain], the [VL Domain], the [TH Domain], and the [ASt Domain2], four 2′-fluoro modifications in the [DH Domain], the [ACH Domain], and the [TH Domain], and four phosphorothioate modifications in the [ACH Domain] and the [TH Domain]. In some embodiments, the TREM comprises the non-naturally occurring modification pattern of Pattern No: 25 in Table 6: 1-m, 2-m, 3-m, 4-m, 5-m, 6-m, 10-m, 11-m, 12-m, 13-m, 14-m, 15-f, 16-m, 17-m, 18-m, 19-f, 20-m, 25-m, 33-f, 35-*, 37-*, 38-*, 41-m, 42-m, 43-m, 44-m, 49-m, 50-m, 52-m, 54-m, 55-*, 56-m, 57-f, 59-m, 61-m, 65-m, 73-m.

A TREM may comprise a non-naturally occurring modification in each of the [ASt Domain1], the [DH Domain], the [ACH Domain], the [VL Domain], the [TH Domain], and the [ASt Domain2]. In some embodiments, the TREM comprises a nucleotide sugar modification in each of the [ASt Domain1], the [DH Domain], the [ACH Domain], the [VL Domain], the [TH Domain], and the [ASt Domain2]. In some embodiments, the TREM comprises an internucleotide modification in each of the [ACH Domain] and the [TH Domain]. In some embodiments, the TREM comprises thirty-six non-naturally occurring modifications. In some embodiments, the TREM comprises twenty-eight 2′-O-methyl modifications. In some embodiments, the TREM comprises four 2′-fluoro modifications. In some embodiments, the TREM comprises four phosphorothioate modifications. In some embodiments, the TREM comprises twenty-eight 2′-O-methyl modifications in the [ASt Domain1], the [DH Domain], the [ACH Domain], the [VL Domain], the [TH Domain], and the [ASt Domain2], four 2′-fluoro modifications in the [ACH Domain], the [VL Domain], and the [TH Domain], and four phosphorothioate modifications in the [ACH Domain] and the [TH Domain]. In some embodiments, the TREM comprises the non-naturally occurring modification pattern of Pattern No: 26 in Table 6: 1-m, 2-m, 3-m, 4-m, 5-m, 6-m, 16-m, 17-m, 18-m, 20-m, 22-m, 23-m, 24-m, 25-m, 33-f, 35-*, 37-*, 38-*, 41-m, 42-m, 43-m, 44-m, 45-m, 46-f, 48-f, 49-m, 50-m, 52-m, 54-m, 55-*, 56-m, 57-f, 59-m, 61-m, 65-m, 73-m.

A TREM may comprise a non-naturally occurring modification in each of the [ASt Domain1], the [DH Domain], the [ACH Domain], the [VL Domain], the [TH Domain], and the [ASt Domain2]. In some embodiments, the TREM comprises a nucleotide sugar modification in each of the [ASt Domain1], the [DH Domain], the [ACH Domain], the [VL Domain], the [TH Domain], and the [ASt Domain2]. In some embodiments, the TREM comprises an internucleotide modification in the [TH Domain]. In some embodiments, the TREM comprises thirty-one non-naturally occurring modifications. In some embodiments, the TREM comprises twenty-eight 2′-O-methyl modifications. In some embodiments, the TREM comprises two 2′-fluoro modifications. In some embodiments, the TREM comprises one phosphorothioate modification. In some embodiments, the TREM comprises twenty-eight 2′-O-methyl modifications in the [ASt Domain1], the [DH Domain], the [ACH Domain], the [VL Domain], the [TH Domain], and the [ASt Domain2], two 2′-fluoro modifications in the [TH Domain], and one phosphorothioate modification in the [TH Domain]. In some embodiments, the TREM comprises the non-naturally occurring modification pattern of Pattern No: 27 in Table 6: 1-m, 2-m, 3-m, 4-m, 5-m, 6-m, 14-m, 15-m, 16-m, 17-m, 18-m, 20-m, 25-m, 41-m, 42-m, 43-m, 44-m, 49-m, 50-m, 52-m, 54-m, 55-*, 56-m, 57-f, 59-m, 61-m, 62-m, 63-f, 64-m, 65-m, 73-m.

A TREM may comprise a non-naturally occurring modification in each of the [ASt Domain1], the [DH Domain], the [ACH Domain], the [VL Domain], the [TH Domain], and the [ASt Domain2]. In some embodiments, the TREM comprises a nucleotide sugar modification in each of the [ASt Domain1], the [DH Domain], the [ACH Domain], the [VL Domain], the [TH Domain], and the [ASt Domain2]. In some embodiments, the TREM comprises an internucleotide modification in each of the [ACH Domain] and the [TH Domain]. In some embodiments, the TREM comprises thirty-three non-naturally occurring modifications. In some embodiments, the TREM comprises twenty-four 2′-O-methyl modifications. In some embodiments, the TREM comprises five 2′-fluoro modifications. In some embodiments, the TREM comprises four phosphorothioate modifications. In some embodiments, the TREM comprises twenty-four 2′-O-methyl modifications in the [ASt Domain1], the [DH Domain], the [ACH Domain], the [VL Domain], the [TH Domain], and the [ASt Domain2], five 2′-fluoro modifications in the [ACH Domain], the [VL Domain], and the [TH Domain], and four phosphorothioate modifications in the [ACH Domain] and the [TH Domain]. In some embodiments, the TREM comprises the non-naturally occurring modification pattern of Pattern No: 28 in Table 6: 1-m, 2-m, 3-m, 4-m, 5-m, 6-m, 16-m, 17-m, 18-m, 20-m, 25-m, 33-f, 35-*, 37-*, 38-*, 41-f, 42-m, 43-m, 44-f, 49-m, 50-m, 52-m, 54-m, 55-*, 56-m, 57-f, 59-m, 61-m, 62-m, 63-f, 64-m, 65-m, 73-m.

A TREM may comprise a non-naturally occurring modification in each of the [ASt Domain1], the [DH Domain], the [ACH Domain], the [VL Domain], the [TH Domain], and the [ASt Domain2]. In some embodiments, the TREM comprises a nucleotide sugar modification in each of the [ASt Domain1], the [DH Domain], the [ACH Domain], the [VL Domain], the [TH Domain], and the [ASt Domain2]. In some embodiments, the TREM comprises an internucleotide modification in each of the [ACH Domain] and the [TH Domain]. In some embodiments, the TREM comprises thirty-five non-naturally occurring modifications. In some embodiments, the TREM comprises twenty-six 2′-O-methyl modifications. In some embodiments, the TREM comprises five 2′-fluoro modifications. In some embodiments, the TREM comprises four phosphorothioate modifications. In some embodiments, the TREM comprises twenty-six 2′-O-methyl modifications in the [ASt Domain1], the [DH Domain], the [ACH Domain], the [VL Domain], the [TH Domain], and the [ASt Domain2], five 2′-fluoro modifications in the [ACH Domain], the [VL Domain], and the [TH Domain], and four phosphorothioate modifications in the [ACH Domain] and the [TH Domain]. In some embodiments, the TREM comprises the non-naturally occurring modification pattern of Pattern No: 29 in Table 6: 1-m, 2-m, 3-m, 4-m, 5-m, 6-m, 14-m, 15-m, 16-m, 17-m, 18-m, 20-m, 25-m, 33-f, 35-*, 37-*, 38-*, 41-f, 42-m, 43-m, 44-f, 49-m, 50-m, 52-m, 54-m, 55-*, 56-m, 57-f, 59-m, 61-m, 62-m, 63-f, 64-m, 65-m, 73-m.

A TREM may comprise a non-naturally occurring modification in each of the [ASt Domain1], the [DH Domain], the [ACH Domain], and the [ASt Domain2]. In some embodiments, the TREM comprises a nucleotide sugar modification in each of the [ASt Domain1], the [DH Domain], the [ACH Domain], and the [ASt Domain2]. In some embodiments, the TREM comprises sixteen non-naturally occurring modifications. In some embodiments, the TREM comprises fifteen 2′-O-methyl modifications. In some embodiments, the TREM comprises one 2′-fluoro modification. In some embodiments, the TREM comprises fifteen 2′-O-methyl modifications in the [ASt Domain1], the [DH Domain], the [ACH Domain], and the [ASt Domain2] and one 2′-fluoro modification in the [ACH Domain]. In some embodiments, the TREM comprises the non-naturally occurring modification pattern of Pattern No: 30 in Table 6: 1-m, 2-m, 3-m, 4-m, 5-m, 6-m, 14-m, 17-m, 18-m, 20-m, 22-m, 25-m, 27-m, 33-f, 71-m, 73-m.

A TREM may comprise a non-naturally occurring modification in each of the [ASt Domain1], the [VL Domain], and the [ASt Domain2]. In some embodiments, the TREM comprises a nucleotide sugar modification in each of the [ASt Domain1], the [VL Domain], and the [ASt Domain2]. In some embodiments, the TREM comprises an internucleotide modification in the [ASt Domain1]. In some embodiments, the TREM comprises fourteen non-naturally occurring modifications. In some embodiments, the TREM comprises eleven 2′-O-methyl modifications. In some embodiments, the TREM comprises two 2′-fluoro modifications. In some embodiments, the TREM comprises one phosphorothioate modification. In some embodiments, the TREM comprises eleven 2′-O-methyl modifications in the [ASt Domain1], the [VL Domain], and the [ASt Domain2], two 2′-fluoro modifications in the [VL Domain], and one phosphorothioate modification in the [ASt Domain1]. In some embodiments, the TREM comprises the non-naturally occurring modification pattern of Pattern No: 31 in Table 6: 1-m*, 2-m, 3-m, 4-m, 5-m, 6-m, 44-m, 45-m, 46-f, 47-f, 48-m, 49-m, 73-m.

A TREM may comprise a non-naturally occurring modification in each of the [ASt Domain1], the [DH Domain], the [ACH Domain], the [VL Domain], the [TH Domain], and the [ASt Domain2]. In some embodiments, the TREM comprises a nucleotide sugar modification in each of the [ASt Domain1], the [DH Domain], the [ACH Domain], the [VL Domain], the [TH Domain], and the [ASt Domain2]. In some embodiments, the TREM comprises an internucleotide modification in each of the [ASt Domain1], the [ACH Domain], and the [TH Domain]. In some embodiments, the TREM comprises forty-two non-naturally occurring modifications. In some embodiments, the TREM comprises thirty-one 2′-O-methyl modifications. In some embodiments, the TREM comprises four 2′-fluoro modifications. In some embodiments, the TREM comprises seven phosphorothioate modifications. In some embodiments, the TREM comprises thirty-one 2′-O-methyl modifications in the [ASt Domain1], the [DH Domain], the [ACH Domain], the [VL Domain], the [TH Domain], and the [ASt Domain2], four 2′-fluoro modifications in the [ACH Domain], the [VL Domain], and the [TH Domain], and seven phosphorothioate modifications in the [ASt Domain1], the [ACH Domain], and the [TH Domain]. In some embodiments, the TREM comprises the non-naturally occurring modification pattern of Pattern No: 32 in Table 6: 1-m*, 2-m*, 3-m, 4-m, 5-m, 6-m, 10-m, 11-m, 12-m, 13-m, 17-m, 18-m, 22-m, 23-m, 24-m, 25-m, 33-f, 35-*, 37-*, 38-*, 41-f, 42-m, 43-m, 44-f, 49-m, 50-m, 51-m, 52-m, 53-m, 54-*, 55-*, 61-m, 62-m, 63-f, 64-m, 65-m, 66-m, 67-m, 68-m, 73-m.

Table 6: Exemplary non-naturally occurring modification patterns of TREMs

TABLE 6
Exemplary non-naturally occurring modification patterns of TREMs

| Pattern | |
|---|---|
| No. | Modification Pattern |
| 1 | 1-m, 14-m, 15-m, 16-m, 17-m, 18-m, 19-*, 20-m, 21-*, 33-f, 34-m, 35-*, 37-*, 38-*, |
| | 41-m, 42-m, 43-m, 44-m, 50-m, 52-m, 73-m |
| 2 | 1-m*, 2-m*, 74-*, 75-m |
| 3 | 1-m*, 2-m*, 52-*, 74-*, 75-m |
| 4 | 1-m, 2-m, 3-m, 4-m, 5-m, 6-m, 13-m, 14-m, 15-m, 16-m, 17-m, 18-m, 19-*, 20-m, |
| | 21-*, 25-m, 33-*, 34-m, 35-*, 37-*, 38-*, 49-m, 50-m, 51-m, 52-m, 53-m, 54-*, 55- |
| | *, 61-m, 65-m, 73-m |
| 5 | 1-m*, 2-m*, 13-f, 14-f, 54-f, 59-m, 75-*, 76-m |
| 6 | 1-m, 13-m, 17-m, 18-m, 25-m, 33-*, 34-m, 35-*, 37-*, 38-*, 41-f, 42-m, 43-m, 44- |
| | f, 49-m, 50-m, 52-m, 54-*, 55-*, 56-m, 57-*, 58-*, 59-m, 61-m, 65-m, 73-m |
| 7 | 1-m, 13-m, 14-m, 15-m, 16-m, 17-m, 18-m, 19-*, 20-m, 21-*, 25-m, 41-f, 42-m, 43- |
| | m, 44-f, 49-m, 50-m, 51-m, 52-m, 53-m, 54-*, 55-*, 61-m, 65-m, 73-m |
| 8 | 1-m, 19-m, 20-m, 21-f, 22-m, 40-f, 41-m, 42-m, 43-m, 73-m |
| 9 | 1-m*, 2-m, 3-m, 4-m, 5-m, 6-m, 27-m, 28-m, 29-f, 30-m, 40-f, 41-m, 42-m, 43-m, |
| | 73-m |
| 10 | 1-m*, 2-m, 3-m, 4-m, 5-m, 6-m, 49-m, 50-m, 51-m, 52-m, 53-m, 61-m, 62-m, 63-f, |
| | 64-m, 65-m, 66-m, 67-m, 68-m, 69-m, 71-m, 72-m |
| 11 | 1-m*, 2-m*, 3-m, 4-m, 5-m, 6-m, 10-m, 11-m, 12-m, 13-m, 22-m, 23-m, 24-m, 25-m, |
| | 49-m, 50-m, 51-m, 52-m, 53-m, 54-m, 61-m, 62-m, 63-f, 64-m, 65-m, 66-m, 67-m, |
| | 68-m, 73-m |
| 12 | 1-m*, 2-m*, 3-m, 4-m, 5-m, 6-m, 16-m, 17-m, 18-m, 20-m, 25-m, 33-f, 35-*, 37-*, |
| | 38-*, 41-m, 44-m, 45-m, 46-f, 47-f, 48-f, 49-m, 50-m, 51-m, 52-m, 53-m, 54-m, 56- |
| | m, 59-m, 73-m |
| 13 | 1-m, 2-m, 3-m, 4-m, 5-m, 6-m, 16-m, 17-m, 18-m, 33-f, 35-*, 37-*, 38-*, 44-m, 45- |
| | m, 46-f, 47-f, 48-f, 56-m, 59-m, 61-m, 62-m, 63-f, 64-m, 65-m, 66-m, 67-m, 68-m, |
| | 73-m |
| 14 | 1-m*, 2-m*, 3-m, 4-m, 5-m, 6-m, 10-m, 11-m, 12-m, 13-m, 14-m, 15-f, 16-m, 17-m, |
| | 18-m, 19-f, 20-m, 22-m, 23-m, 24-m, 25-m, 33-f, 35-*, 37-*, 38-*, 44-m, 45-m, 46-f, |
| | 47-f, 48-f, 56-m, 59-m, 61-m, 62-m, 63-f, 64-m, 65-m, 66-m, 67-m, 68-m, 73-m |
| 15 | 1-m*, 2-m*, 3-m, 4-m, 5-m, 6-m, 16-m, 17-m, 18-m, 20-m, 25-m, 33-f, 35-*, 37-*, |
| | 38-*, 41-m, 44-m, 45-m, 46-f, 47-f, 48-f, 56-m, 59-m, 73-m |
| 16 | 1-m, 17-m, 18-m, 50-m, 52-m, 73-m |
| 17 | 1-m, 14-m, 15-f, 16-m, 17-m, 18-m, 19-f, 20-m, 54-m*, 55-*, 56-m, 57-m, 59-m, 73- |
| | m |
| 18 | 1-m, 10-m, 13-m, 17-*, 18-m, 19-*, 20-m, 25-m, 29-m, 33-f, 35-*, 37-*, 38-*, 41-f*, |
| | 43-m, 44-f, 46-*, 50-m, 52-m, 54-*, 55-*, 56-m, 57-*, 62-m, 63-f, 65-m, 71-m, 73-m |
| 19 | 1-m, 61-m, 62-m, 64-m, 65-m, 66-m, 67-m, 68-m, 69-m, 70-f, 71-m, 72-m, 73-m |
| 20 | 1-m, 14-m, 15-f, 16-m, 17-m, 18-m, 19-f, 20-m, 33-f, 35-*, 37-*, 38-*, 41-m, 42-m, |
| | 43-m, 44-m, 50-m, 52-m, 73-m |
| 21 | 1-m, 2-m, 3-m, 4-m, 5-m, 6-m, 14-m, 15-m, 16-m, 17-m, 18-m, 20-m, 25-m, 41-m, |
| | 42-m, 43-m, 44-m, 49-m, 50-m, 52-m, 54-m, 55-*, 56-m, 57-f, 59-m, 61-m, 65-m, |
| | 73-m |
| 22 | 1-m, 2-m, 3-m, 4-m, 5-m, 6-m, 14-m, 15-m, 16-m, 17-m, 18-m, 20-m, 25-m, 33-f, |
| | 35-*, 37-*, 38-*, 41-m, 42-m, 43-m, 44-f, 49-m, 50-m, 52-m, 54-m, 55-*, 56-m, 57-f, |
| | 59-m, 61-m, 65-m, 73-m |
| 23 | 1-m, 2-m, 3-m, 4-m, 5-m, 6-m, 16-m, 17-m, 18-m, 20-m, 25-m, 33-f, 35-*, 37-*, 38- |
| | *, 41-m, 42-m, 43-m, 44-m, 49-m, 50-m, 52-m, 54-*, 55-*, 61-m, 65-m, 73-m |
| 24 | 1-m, 2-m, 3-m, 4-m, 5-m, 6-m, 10-m, 11-m, 12-m, 13-m, 16-m, 17-m, 18-m, 20-m, |
| | 25-m, 33-f, 35-*, 37-*, 38-*, 41-f, 42-m, 43-m, 44-f, 49-m, 50-m, 52-m, 54-m, 55-*, |
| | 56-m, 57-f, 59-m, 61-m, 65-m, 73-m |
| 25 | 1-m, 2-m, 3-m, 4-m, 5-m, 6-m, 10-m, 11-m, 12-m, 13-m, 14-m, 15-f, 16-m, 17-m, |
| | 18-m, 19-f, 20-m, 25-m, 33-f, 35-*, 37-*, 38-*, 41-m, 42-m, 43-m, 44-m, 49-m, 50- |
| | m, 52-m, 54-m, 55-*, 56-m, 57-f, 59-m, 61-m, 65-m, 73-m |
| 26 | 1-m, 2-m, 3-m, 4-m, 5-m, 6-m, 16-m, 17-m, 18-m, 20-m, 22-m, 23-m, 24-m, 25-m, |
| | 33-f, 35-*, 37-*, 38-*, 41-m, 42-m, 43-m, 44-m, 45-m, 46-f, 48-f, 49-m, 50-m, 52-m, |
| | 54-m, 55-*, 56-m, 57-f, 59-m, 61-m, 65-m, 73-m |
| 27 | 1-m, 2-m, 3-m, 4-m, 5-m, 6-m, 14-m, 15-m, 16-m, 17-m, 18-m, 20-m, 25-m, 41-m, |
| | 42-m, 43-m, 44-m, 49-m, 50-m, 52-m, 54-m, 55-*, 56-m, 57-f, 59-m, 61-m, 62-m, |
| | 63-f, 64-m, 65-m, 73-m |
| 28 | 1-m, 2-m, 3-m, 4-m, 5-m, 6-m, 16-m, 17-m, 18-m, 20-m, 25-m, 33-f, 35-*, 37-*, 38- |
| | *, 41-f, 42-m, 43-m, 44-f, 49-m, 50-m, 52-m, 54-m, 55-*, 56-m, 57-f, 59-m, 61-m, |
| | 62-m, 63-f, 64-m, 65-m, 73-m |
| 29 | 1-m, 2-m, 3-m, 4-m, 5-m, 6-m, 14-m, 15-m, 16-m, 17-m, 18-m, 20-m, 25-m, 33-f, |
| | 35-*, 37-*, 38-*, 41-f, 42-m, 43-m, 44-f, 49-m, 50-m, 52-m, 54-m, 55-*, 56-m, 57-f, |
| | 59-m, 61-m, 62-m, 63-f, 64-m, 65-m, 73-m |
| 30 | 1-m, 2-m, 3-m, 4-m, 5-m, 6-m, 14-m, 17-m, 18-m, 20-m, 22-m, 25-m, 27-m, 33-f, |
| | 71-m, 73-m |
| 31 | 1-m*, 2-m, 3-m, 4-m, 5-m, 6-m, 44-m, 45-m, 46-f, 47-f, 48-m, 49-m, 73-m |
| 32 | 1-m*, 2-m*, 3-m, 4-m, 5-m, 6-m, 10-m, 11-m, 12-m, 13-m, 17-m, 18-m, 22-m, 23-m, |
| | 24-m, 25-m, 33-f, 35-*, 37-*, 38-*, 41-f, 42-m, 43-m, 44-f, 49-m, 50-m, 51-m, 52-m, |
| | 53-m, 54-*, 55-*, 61-m, 62-m, 63-f, 64-m, 65-m, 66-m, 67-m, 68-m, 73-m |

### TREM, TREM Core Fragment and TREM Fragment Fusions

In an embodiment, a TREM, a TREM core fragment or a TREM fragment disclosed herein comprises an additional moiety, e.g., a fusion moiety. In an embodiment, the fusion moiety can be used for purification, to alter folding of the TREM, TREM core fragment or TREM fragment, or as a targeting moiety. In an embodiment, the fusion moiety can comprise a tag, a linker, can be cleavable or can include a binding site for an enzyme. In an embodiment, the fusion moiety can be disposed at the N terminal of the TREM or at the C terminal of the TREM, TREM core fragment or TREM fragment. In an embodiment, the fusion moiety can be encoded by the same or different nucleic acid molecule that encodes the TREM, TREM core fragment or TREM fragment.

### TREM Consensus Sequence

In an embodiment, a TREM disclosed herein comprises a consensus sequence provided herein.

In an embodiment, a TREM disclosed herein comprises a consensus sequence of Formula I _{ZZZ}, wherein zzz indicates any of the twenty amino acids and Formula I corresponds to all species.

In an embodiment, a TREM disclosed herein comprises a consensus sequence of Formula II _{ZZZ}, wherein zzz indicates any of the twenty amino acids and Formula II corresponds to mammals.

In an embodiment, a TREM disclosed herein comprises a consensus sequence of Formula III _{ZZZ}, wherein zzz indicates any of the twenty amino acids and Formula III corresponds to humans.

In an embodiment, zzz indicates any of the twenty amino acids: alanine, arginine, asparagine, aspartate, cysteine, glutamine, glutamate, glycine, histidine, isoleucine, methionine, leucine, lysine, phenylalanine, proline, serine, threonine, tryptophan, tyrosine, or valine.

In an embodiment, a TREM disclosed herein comprises a property selected from the following:
a) under physiological conditions residue R₀ forms a linker region, e.g., a Linker 1 region;
b) under physiological conditions residues R₁-R₂-R₃-R₄-R₅-R₆-R₇ and residues R₆₅-R₆₆-R₆₇-R₆₈-R₆₉-R₇₀-R₇₁ form a stem region, e.g., an AStD stem region;
c) under physiological conditions residues R₈-R₉ forms a linker region, e.g., a Linker 2 region;
d) under physiological conditions residues —R₁₀-R₁₁-R₁₂-R₁₃-R₁₄ R₁₅-R₁₆-R₁₇-R₁₈-R₁₉-R₂₀-R₂₁-R₂₂-R₂₃-R₂₄-R₂₅-R₂₆-R₂₇-R₂₈ form a stem-loop region, e.g., a D arm Region;
e) under physiological conditions residue —R₂₉ forms a linker region, e.g., a Linker 3 Region;
f) under physiological conditions residues —R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆ form a stem-loop region, e.g., an AC arm region;
g) under physiological conditions residue —[R₄₇]ₓ comprises a variable region, e.g., as described herein;
h) under physiological conditions residues —R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄ form a stem-loop region, e.g., a T arm Region; or
i) under physiological conditions residue R₇₂ forms a linker region, e.g., a Linker 4 region.

### Alanine TREM Consensus Sequence

In an embodiment, a TREM disclosed herein comprises the sequence of Formula I_{ALA}(SEQ ID NO: 562),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆—[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂, wherein R is a ribonucleotide residue and the consensus for Ala is: R₀=absent; R₁₄, R₅₇= are independently A or absent; R₂₆=A, C, G or absent; R₅, R₆, R₁₅, R₁₆, R₂₁, R₃₀, R₃₁, R₃₂, R₃₄, R₃₇, R₄₁, R₄₂, R₄₃, R₄₄, R₄₅, R₄₈, R₄₉, R₅₀, R₅₈, R₅₉, R₆₃, R₆₄, R₆₆, R₆₇= are independently N or absent; R₁₁, R₃₅, R₆₅= are independently A, C, U or absent; R₁, R₉, R₂₀, R₃₈, R₄₀, R₅₁, R₅₂, R₅₆= are independently A, G or absent; R₇, R₂₂, R₂₅, R₂₇, R₂₉, R₄₆, R₅₃, R₇₂= are independently A, G, U or absent; R₂₄, R₆₉= are independently A, U or absent; R₇₀, R₇₁= are independently C or absent; R₃, R₄= are independently C, G or absent; R₁₂, R₃₃, R₃₆, R₆₂, R₆₈= are independently C, G, U or absent; R₁₃, R₁₇, R₂₈, R₃₉, R₅₅, R₆₀, R₆₁= are independently C, U or absent; R₁₀, R₁₉, R₂₃= are independently G or absent; R₂=G, U or absent; R₈, R₁₈, R₅₄= are independently U or absent; [R₄₇]ₓ═N or absent; wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271),
   provided that the TREM has one or both of the following properties: no more than 15% of the residues are N; or no more than 20 residues are absent.

In an embodiment, a TREM disclosed herein comprises the sequence of Formula II_{ALA} (SEQ ID NO: 563),

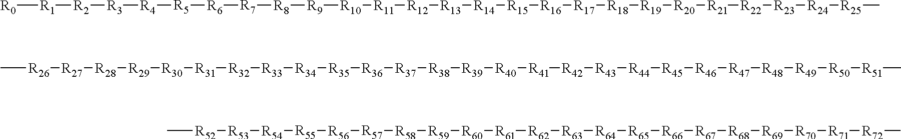

wherein R is a ribonucleotide residue and the consensus for Ala is:
R₀, R₁₈= are absent
R₁₄, R₂₄, R₅₇= are independently A or absent;
R₁₅, R₂₆, R₆₄= are independently A, C, G or absent;
R₁₆, R₃₁, R₅₀, R₅₉= are independently N or absent;
R₁, R₃₂, R₃₇, R₄₁, R₄₃, R₄₅, R₄₉, R₆₅, R₆₆= are independently A, C, U or absent;
R₁, R₅, R₉, R₂₅, R₂₇, R₃₈, R₄₀, R₄₆, R₁₁, R₅₆= are independently A, G or absent;
R₇, R₂, R₂₉, R₄₂, R₄₄, R₅₃, R₆₃, R₇₂= are independently A, G, U or absent;
R₆, R₃₅, R₆₉= are independently A, U or absent;
R₅₅, R₆₀, R₇₀, R₇₁= are independently C or absent;
R₃=C, G or absent;
R₁₂, R₃₆, R₄₈= are independently C, G, U or absent;
R₁₃, R₁₇, R₂₈, R₃₀, R₃₄, R₃₉, R₅₈, R₆₁, R₆₂, R₆₇, R₆₈= are independently C, U or absent;
R₄, R₁₀, R₁₉, R₂₀, R₂₃, R₅₂= are independently G or absent;
R₂, R₈, R₃₃= are independently G, U or absent;
R₂₁, R₅₄= are independently U or absent;
[R₄₇]ₓ═N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III_{ALA}(SEQ ID NO: 564),

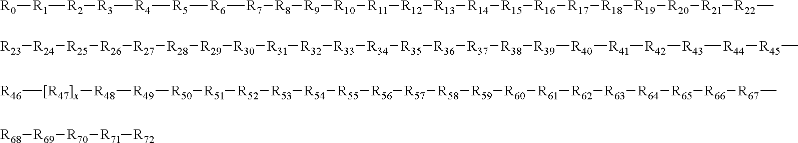

wherein R is a ribonucleotide residue and the consensus for Ala is:
R₀, R₁₈= are absent;
R₁₄, R₂₄, R₅₇, R₇₂= are independently A or absent;
R₁₅, R₂₆, R₆₄= are independently A, C, G or absent;
R₁₆, R₃, R₅₀= are independently N or absent;
R₁₁, R₃₂, R₃₇, R₄₁, R₄₃, R₄₅, R₄₉, R₆₅, R₆₆= are independently A, C, U or absent;
R₅, R₉, R₂₅, R₂₇, R₃₈, R₄₀, R₄₆, R₅₁, R₅₆= are independently A, G or absent;
R₇, R₂₂, R₂₉, R₄₂, R₄₄, R₅₃, R₆₃= are independently A, G, U or absent;
R₆, R₃₅= are independently A, U or absent;
R₅₅, R₆₀, R₆₁, R₇₀, R₇₁= are independently C or absent;
R₁₂, R₄₈, R₅₉= are independently C, G, U or absent;
R₁₃, R₁₇, R₂₈, R₃₀, R₃₄, R₃₉, R₅₈, R₆₂, R₆₇, R₆₈= are independently C, U or absent;
R₁, R₂, R₃, R₄, R₁₀, R₁₉, R₂₀, R₂₃, R₅₂= are independently G or absent;
R₃₃, R₃₆= are independently G, U or absent;
R₈, R₂₁, R₅₄, R₆₉= are independently U or absent;
[R₄₇]ₓ═N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Arginine TREM Consensus Sequence

In an embodiment, a TREM disclosed herein comprises the sequence of Formula I ARG (SEQ ID NO: 565),

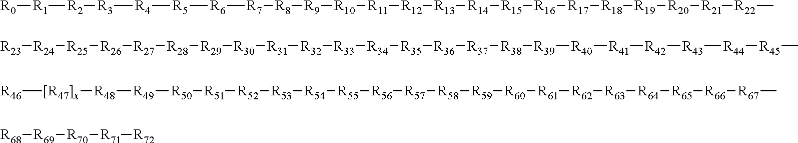

wherein R is a ribonucleotide residue and the consensus for Arg is:
R₅₇=A or absent;
R₉, R₂₇= are independently A,C,G or absent;
R₁, R₂, R₃, R₄, R₅, R₆, R₇, R₁₁, R₁₂, R₁₆, R₂₁, R₂₂, R₂₃, R₂₅, R₂₆, R₂₉, R₃₀, R₃₁, R₃₂, R₃₃, R₃₄, R₃₇, R₄₂, R₄₄, R₄₅, R₄₆, R₄₅, R₄₉, R₅₀, R₅₁, R₅₅, R₆₂, R₆₃, R₆₄, R₆₅, R₆₆, R₆₇, R₆₅, R₆₉, R₇₀, R₇₁= are independently N or absent;
R₁₃, R₁₇, R₄₁= are independently A,C,U or absent;
R₁₉, R₂₀, R₂₄, R₄₀, R₅₆= are independently A,G or absent;
R₁₄, R₁₅, R₇₂= are independently A,G,U or absent;
R₁₈=A,U or absent;
R₃₈═C or absent;
R₃₅, R₄₃, R₆₁= are independently C,G,U or absent;
R₂₈, R₅₅, R₅₉, R₆₀= are independently C,U or absent;
R₀, R₁₀, R₅₂= are independently G or absent;
R₈, R₃₉= are independently G,U or absent;
R₃₆, R₅₃, R₅₄= are independently U or absent;
[R₄₇]ₓ═N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II _{ARG} (SEQ ID NO: 566),

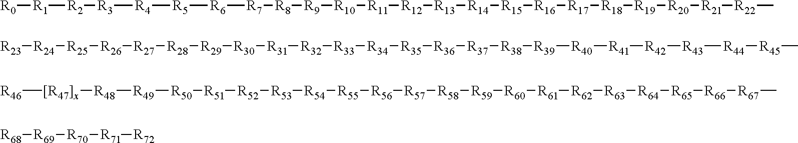

wherein R is a ribonucleotide residue and the consensus for Arg is:
R₁₈=absent;
R₂₄, R₅₇= are independently A or absent;
R₄₁=A,C or absent;
R₃, R₇, R₃₄, R₅₀= are independently A,C,G or absent;
R₂, R₅, R₆, R₁₂, R₂₆, R₃₂, R₃₇, R₄₄, R₅₈, R₆₆, R₆₇, R₆₈, R₇₀= are independently N or absent;
R₄₉, R₇₁= are independently A,C,U or absent;
R₁, R₁₅, R₁₉, R₂₅, R₂₇, R₄₀, R₄₅, R₄₆, R₅₆, R₇₂= are independently A,G or absent;
R₁₄, R₂₉, R₆3= are independently A,G,U or absent;
R₁₆, R₂= are independently A,U or absent;
R₃₅, R₆₁= are independently C or absent;
R₃₃, R₄₈= are independently C,G or absent;
R₄, R₉, R₁₁, R₄₃, R₆₂, R₆₄, R₆₉= are independently C,G,U or absent;
R₁₃, R₂₂, R₂₈, R₃₀, R₃₁, R₃₅, R₅₅, R₆₀, R₆₅= are independently C,U or absent;
R₀, R₁₀, R₂₀, R₂3, R₅₁, R₅₂= are independently G or absent;
R₈, R₃₉, R₄₂= are independently G,U or absent;
R₁₇, R₃₆, R₅₃, R₅₄, R₅₉= are independently U or absent;
[R₄₇]ₓ═N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III ARG (SEQ ID NO: 567),

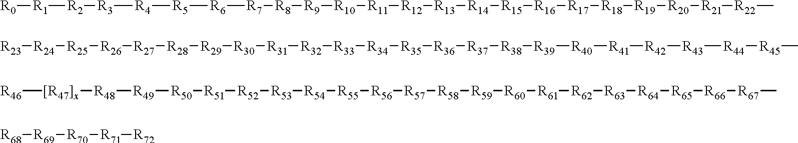

wherein R is a ribonucleotide residue and the consensus for Arg is:
R₁₈= is absent;
R₁₅, R₂₁, R₂₄, R₄₁, R₅₇= are independently A or absent;
R₃₄, R₄₄= are independently A,C or absent;
R₃, R₅, R₅₈= are independently A,C,G or absent;
R₂, R₆, R₆₆, R₇₀= are independently N or absent;
R₃₇, R₄₉= are independently A,C,U or absent;
R₁, R₂₅, R₂₉, R₄₀, R₄₅, R₄₆, R₅₀= are independently A,G or absent;
R₁₄, R₆₃, R₆₈= are independently A,G,U or absent;
R₁₆=A,U or absent;
R₃₅, R₆₁= are independently C or absent;
R₇, R₁₁, R₁₂, R₂₆, R₄₈= are independently C,G or absent;
R₆₄, R₆₇, R₆₉= are independently C,G,U or absent;
R₄, R₁₃, R₂₂, R₂₈, R₃₀, R₃₁, R₃₅, R₄₃, R₅₅, R₆₀, R₆₂, R₆₅, R₇₁= are independently C,U or absent;
R₀, R₁₀, R₁₉, R₂₀, R₂₃, R₂₇, R₃₃, R₅₁, R₅₂, R₅₆, R₇₂= are independently G or absent;
R₈, R₉, R₃₂, R₃₉, R₄₂= are independently G,U or absent;
R₁₇, R₃₆, R₅₃, R₅₄, R₅₉= are independently U or absent;
[R₄₇]ₓ═N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Asparagine TREM Consensus Sequence

In an embodiment, a TREM disclosed herein comprises the sequence of Formula I ASN (SEQ ID NO: 568),

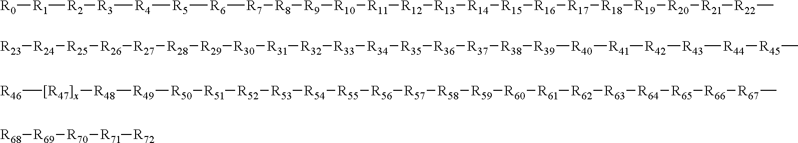

wherein R is a ribonucleotide residue and the consensus for Asn is:
R₀, R₁₈= are absent;
R₄₁=A or absent;
R₁₄, R₄₅, R₅₆= are independently A,C,G or absent;
R₂, R₄, R₅, R₆, R₁₂, R₁₇, R₂₆, R₂₉, R₃₀, R₃₁, R₄₄, R₄₅, R₄₆, R₄₉, R₅₀, R₅₅, R₆₂, R₆₃, R₆₅, R₆₆, R₆₇, R₆₅, R₇₀, R₇₁= are independently N or absent;
R₁₁, R₁₃, R₂₂, R₄₂, R₅₅, R₅₉= are independently A,C,U or absent;
R₉, R₁₅, R₂₄, R₂₇, R₃₄, R₃₇, R₅₁, R₇₂= are independently A,G or absent;
R₁, R₇, R₂₅, R₆₉= are independently A,G,U or absent;
R₄₀, R₅₇= are independently A,U or absent;
R₆₀=C or absent;
R₃₃=C,G or absent;
R₂₁, R₃₂, R₄₃, R₆₄= are independently C,G,U or absent;
R₃, R₁₆, R₂₅, R₃₅, R₃₆, R₆₁= are independently C,U or absent;
R₁₀, R₁₉, R₂₀, R₅₂= are independently G or absent;
R₅₄=G,U or absent;
R₈, R₂₃, R₃₅, R₃₉, R₅₃= are independently U or absent;
[R₄₇]ₓ═N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II _{ASN} (SEQ ID NO: 569),

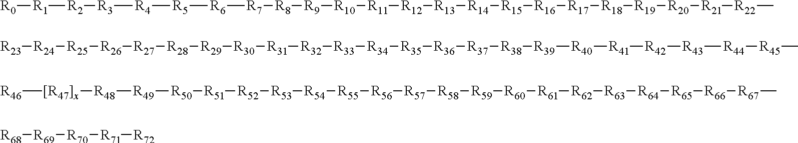

wherein R is a ribonucleotide residue and the consensus for Asn is:
R₀, R₁₈= are absent
R₂₄, R₄₁, R₄₆, R₆₂= are independently A or absent;
R₅₉=A,C or absent;
R₁₄, R₅₆, R₆₆= are independently A,C,G or absent;
R₁₇, R₂₉= are independently N or absent;
R₁₁, R₂₆, R₄₂, R₅₅= are independently A,C,U or absent;
R₁, R₉, R₁₂, R₁₅, R₂₅, R₃₄, R₃₇, R₄₈, R₅₁, R₆₇, R₆₈, R₆₉, R₇₀, R₇₂= are independently A,G or absent;
R₄₄, R₄₅, R₅₈= are independently A,G,U or absent;
R₄₀, R₅₇= are independently A,U or absent;
R₅, R₂₈, R₆₀= are independently C or absent;
R₃₃, R₆₅= are independently C,G or absent;
R₂₁, R₄₃, R₇₁= are independently C,G,U or absent;
R₃, R₆, R₁₃, R₂₂, R₃₂, R₃₅, R₃₆, R₆₁, R₆₃, R₆₄= are independently C,U or absent;
R₇, R₁₀, R₁₉, R₂₀, R₂₇, R₄₉, R₅₂= are independently G or absent;
R₅₄=G,U or absent;
R₂, R₄, R₈, R₁₆, R₂₃, R₃₀, R₃₁, R₃₈, R₃₉, R₅₀, R₅₃= are independently U or absent;
[R₄₇]ₓ═N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III ASN (SEQ ID NO: 570),

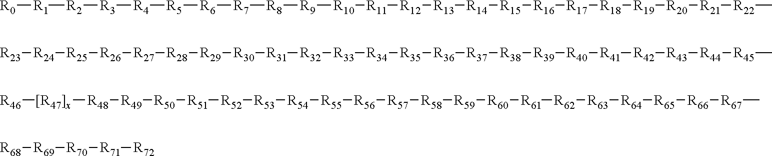

wherein R is a ribonucleotide residue and the consensus for Asn is:
R₀, R₁₈=are absent
R₂₄, R₄₀, R₄₁, R₄₆, R₆₂= are independently A or absent;
R₅₉=A,C or absent;
R₁₄, R₅₆, R₆₆= are independently A,C,G or absent;
R₁₁, R₂₆, R₄₂, R₅₅= are independently A,C,U or absent;
R₁, R₉, R₁₂, R₁₅, R₃₄, R₃₇, R₄₈, R₅₁, R₆₇, R₆₈, R₆₉, R₇₀= are independently A,G or absent;
R₄₄, R₄₅, R₅₈= are independently A,G,U or absent;
R₅₇=A,U or absent;
R₅, R₂₅, R₆₀= are independently C or absent;
R₃₃, R₆₅= are independently C,G or absent;
R₁₇, R₂₁, R₂₉= are independently C,G,U or absent;
R₃, R₆, R₁₃, R₂₂, R₃₂, R₃₅, R₃₆, R₄₃, R₆₁, R₆₃, R₆₄, R₇₁= are independently C,U or absent;
R₇, R₁₀, R₁₉, R₂₀, R₂₅, R₂₇, R₄₉, R₅₂, R₇₂= are independently G or absent;
R₅₄=G,U or absent;
R₂, R₄, R₈, R₁₆, R₂₃, R₃₀, R₃₁, R₃₈, R₃₉, R₅₀, R₅₃= are independently U or absent;
[R₄₇]ₓ═N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Aspartate TREM Consensus Sequence

In an embodiment, a TREM disclosed herein comprises the sequence of Formula I _{ASP} (SEQ ID NO: 571),

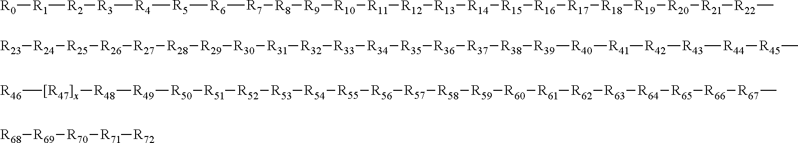

wherein R is a ribonucleotide residue and the consensus for Asp is:
R₀=absent
R₂₄, R₇₁= are independently A,C or absent;
R₃₃, R₄₆= are independently A,C,G or absent;
R₂, R₃, R₄, R₅, R₆, R₁₂, R₁₆, R₂, R₂₆, R₂₉, R₃, R₃₂, R₄₄, R₄₈, R₄₉, R₅₈, R₆₅, R₆₄, R₆₆, R₆₇, R₆₅, R₆₉= are
independently N or absent;
R₁₃, R₂₁, R₃₄, R₄₁, R₅₇, R₆₅= are independently A,C,U or absent;
R₉, R₁₀, R₁₄, R₁₅, R₂₀, R₂₇, R₃₇, R₄₀, R₅₁, R₅₆, R₇₂= are independently A,G or absent;
R₇, R₂₅, R₄₂= are independently A,G,U or absent;
R₃₉=C or absent;
R₅₀, R₆₂= are independently C,G or absent;
R₃₀, R₄₃, R₄₅, R₅₅, R₇₀= are independently C,G,U or absent;
R₈, R₁₁, R₁₇, R₁₅, R₂₅, R₃₅, R₅₃, R₅₉, R₆₀, R₆₁= are independently C,U or absent;
R₁₉, R₅₂= are independently G or absent;
R₁=G,U or absent;
R₂₃, R₃₆, R₃₈, R₅₄= are independently U or absent;
[R₄₇]ₓ═N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II A_{SP}(SEQ ID NO: 572),

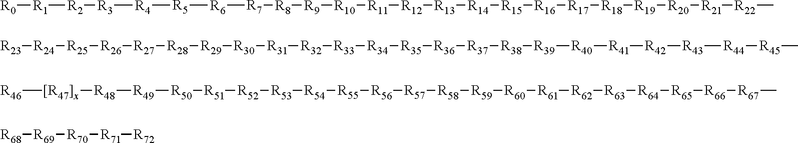

wherein R is a ribonucleotide residue and the consensus for Asp is:
R₀, R₁₇, R₁₈, R₂₃= are independently absent;
R₉, R₄₀= are independently A or absent;
R₂₄, R₇₁= are independently A,C or absent;
R₆₇, R₆₈= are independently A,C,G or absent;
R₂, R₆, R₆₆= are independently N or absent;
R₅₇, R₆₃= are independently A,C,U or absent;
R₁₀, R₁₄, R₂₇, R₃₃, R₃₇, R₄₄, R₄₆, R₅₁, R₅₆, R₆₄, R₇₂= are independently A,G or absent;
R₇, R₁₂, R₂₆, R₆₅= are independently A,U or absent;
R₃₉, R₆₁, R₆₂= are independently C or absent;
R₃, R₃₁, R₄₅, R₇₀= are independently C,G or absent;
R₄, R₅, R₂₉, R₄₃, R₅₅= are independently C,G,U or absent;
R₈, R₁₁, R₁₃, R₃₀, R₃₂, R₃₄, R₃₅, R₄₁, R₄₈, R₅₃, R₅₉, R₆₀= are independently C,U or absent;
R₁₅, R₁₉, R₂₀, R₂₅, R₄₂, R₅₀, R₅₂= are independently G or absent;
R₁, R₂₂, R₄₉, R₅₈, R₆₉= are independently G,U or absent;
R₁₆, R₂₁, R₂₈, R₃₆, R₃₈, R₅₄= are independently U or absent;
[R₄₇]ₓ═N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III ASP (SEQ ID NO: 573),

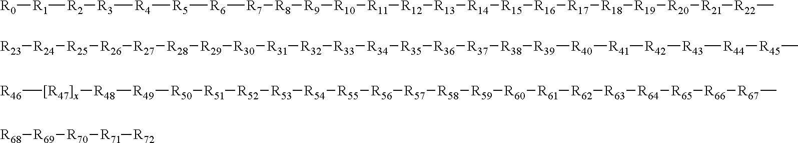

wherein R is a ribonucleotide residue and the consensus for Asp is:
R₀, R₁₇, R₁₅, R₂₃= are absent
R₉, R₁₂, R₄₀, R₆₅, R₇₁= are independently A or absent;
R₂, R₂₄, R₅₇= are independently A,C or absent;
R₆, R₁₄, R₂₇, R₄₆, R₅₁, R₅₆, R₆₄, R₆₇, R₆₈= are independently A,G or absent;
R₃, R₃₁, R₃₅, R₃₉, R₆₁, R₆₂= are independently C or absent;
R₆₆=C,G or absent;
R₅, R₈, R₂₉, R₃₀, R₃₂, R₃₄, R₄₁, R₄₃, R₄₈, R₅₅, R₅₉, R₆₀, R₆₃= are independently C,U or absent;
R₁₀, R₁₅, R₁₉, R₂₀, R₂₅, R₃₃, R₃₇, R₄₂, R₄₄, R₄₅, R₄₉, R₅₀, R₅₂, R₆₉, R₇₀, R₇₂= are independently G or
absent;
R₂₂, R₅₈= are independently G,U or absent;
R₁, R₄, R₇, R₁₁, R₁₃, R₁₆, R₂₁, R₂₆, R₂₈, R₃₆, R₃₈, R₅₃, R₅₄= are independently U or absent;
[R₄₇]ₓ═N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Cysteine TREM Consensus Sequence

In an embodiment, a TREM disclosed herein comprises the sequence of Formula I _{CYS} (SEQ ID NO: 574),

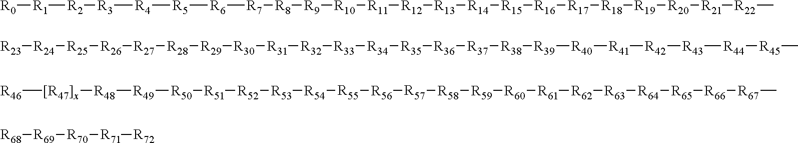

wherein R is a ribonucleotide residue and the consensus for Cys is:
R₀=absent
R₁₄, R₃₉, R₅₇= are independently A or absent;
R₄₁=A,C or absent;
R₁₀, R₁₅, R₂₇, R₃₃, R₆₂= are independently A,C,G or absent;
R₃, R₄, R₅, R₆, R₁₂, R₁₃, R₁₆, R₂₄, R₂₆, R₂₉, R₃₀, R₃₁, R₃₂, R₃₄, R₄₂, R₄₄, R₄₅, R₄₆, R₄₅, R₄₉, R₅₅, R₆₃, R₆₄, R₆₆, R₆₇, R₆₅, R₆₉, R₇₀= are independently N or absent;
R₆₅=A,C,U or absent;
R₉, R₂₅, R₃₇, R₄₀, R₅₂, R₅₆= are independently A,G or absent;
R₇, R₂₀, R₅₁= are independently A,G,U or absent;
R₁₈, R₃₈, R₅₅= are independently C or absent;
R₂═C, G or absent;
R₂₁, R₂₅, R₄₃, R₅₀= are independently C,G,U or absent;
R₁₁, R₂₂, R₂₃, R₃₅, R₃₆, R₅₉, R₆₀, R₆₁, R₇₁, R₂= are independently C,U or absent;
R₁, R₁₉= are independently G or absent;
R₁₇=G,U or absent;
R₈, R₅₃, R₅₄= are independently U or absent;
[R₄₇]ₓ═N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II _{CYS} (SEQ ID NO: 575),

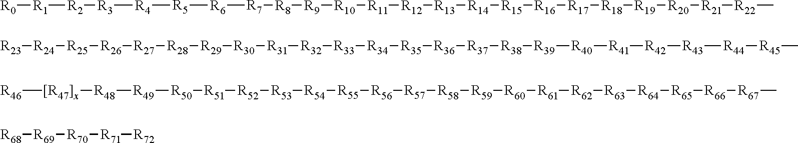

wherein R is a ribonucleotide residue and the consensus for Cys is:
R₀, R₁₈, R₂₃= are absent;
R₁₄, R₂₄, R₂₆, R₂₉, R₃₉, R₄₁, R₄₅, R₅₇= are independently A or absent;
R₄₄=A,C or absent;
R₂₇, R₆₂= are independently A,C,G or absent;
R₁₆=A,C,G,U or absent;
R₃₀, R₇₀= are independently A,C,U or absent;
R₅, R₇, R₉, R₂₅, R₃₄, R₃₇, R₄₀, R₄₆, R₅₂, R₅₆, R₅₈, R₆₆= are independently A,G or absent;
R₂₀, R₅₁= are independently A,G,U or absent;
R₃₅, R₃₈, R₄₃, R₅₅, R₆₉= are independently C or absent;
R₂, R₄, R₁₅= are independently C,G or absent;
R₁₃═C,G,U or absent;
R₆, R₁₁, R₂₅, R₃₆, R₄₈, R₄₉, R₅₀, R₆₀, R₆₁, R₆₇, R₆₈, R₇₁, R₂₂= are independently C,U or absent;
R₁, R₃, R₁₀, R₁₉, R₃₃, R₆₃= are independently G or absent;
R₈, R₁₇, R₂₁, R₆₄= are independently G,U or absent;
R₁₂, R₂₂, R₃₁, R₃₂, R₄₂, R₅₃, R₅₄, R₆₅= are independently U or absent;
R₅₉=U, or absent;
[R₄₇]ₓ═N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III _{CYS} (SEQ ID NO: 576),

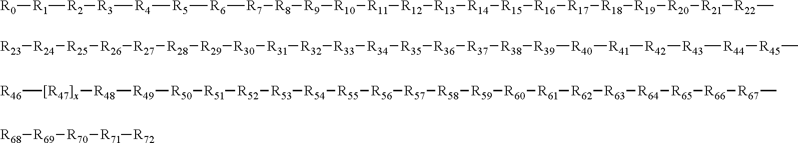

wherein R is a ribonucleotide residue and the consensus for Cys is:
R₀, R₁₅, R₂₃= are absent
R₁₄, R₂₄, R₂₆, R₂₉, R₃₄, R₃₉, R₄₁, R₄₅, R₅₇, R₅₈= are independently A or absent;
R₄₄, R₇₀= are independently A,C or absent;
R₆₂=A,C,G or absent;
R₁₆=N or absent;
R₅, R₇, R₉, R₂₀, R₄₀, R₄₆, R₅₁, R₅₂, R₅₆, R₆₆= are independently A,G or absent;
R₂₈, R₃₅, R₃₈, R₄₃, R₅₅, R₆₇, R₆₉= are independently C or absent;
R₄, R₁₅= are independently C,G or absent;
R₆, R₁₁, R₁₃, R₃₀, R₄₈, R₄₉, R₅₀, R₆₀, R₆₁, R₆₈, R₇₁, R₇₂= are independently C,U or absent;
R₁, R₂, R₃, R₁₀, R₁₉, R₂₅, R₂₇, R₃₃, R₃₇, R₆₃= are independently G or absent;
R₈, R₂₁, R₆₄= are independently G,U or absent;
R₁₂, R₁₇, R₂₂, R₃₁, R₃₂, R₃₆, R₄₂, R₅₃, R₅₄, R₅₉, R₆₅= are independently U or absent;
[R₄₇]ₓ═N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Glutamine TREM Consensus Sequence

In an embodiment, a TREM disclosed herein comprises the sequence of Formula I _{GLN} (SEQ ID NO: 577),

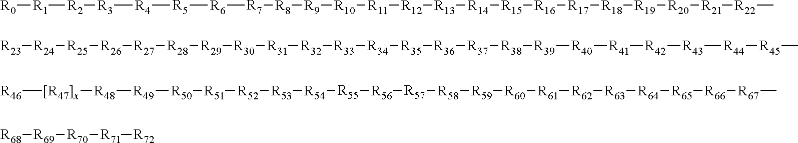

wherein R is a ribonucleotide residue and the consensus for Gln is:
R₀, R₁₈= are absent;
R₁₄, R₂₄, R₅₇= are independently A or absent;
R₉, R₂₆, R₂₇, R₃₃, R₅₆= are independently A,C,G or absent;
R₂, R₄, R₅, R₆, R₁₂, R₁₃, R₁₆, R₂₁, R₂₂, R₂₅, R₂₉, R₃₀, R₃₁, R₃₂, R₃₄, R₄₁, R₄₂, R₄₄, R₄₅, R₄₆, R₄₅, R₄₉, R₅₀, R₅₅, R 62, R₆₃, R₆₆, R₆₇, R₆₈, R₆₉, R₇₀= are independently N or absent;
R₁₇, R₂₃, R₄₃, R₆₅, R₇₁= are independently A,C,U or absent;
R₁₅, R₄₀, R₅₁, R₅₂= are independently A,G or absent;
R₁, R₇, R₇₂= are independently A,G,U or absent;
R₃, R₁₁, R₃₇, R₆₀, R₆₄= are independently C,G,U or absent;
R₂₈, R₃₅, R₅₅, R₅₉, R₆₁= are independently C,U or absent;
R₁₀, R₁₉, R₂₀= are independently G or absent;
R₃₉=G,U or absent;
R₈, R₃₆, R₃₈, R₅₃, R₅₄= are independently U or absent;
[R₄₇]ₓ═N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II _{GLN}(SEQ ID NO: 578),

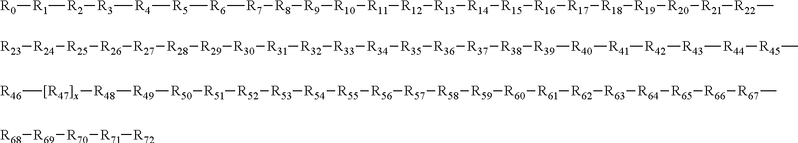

wherein R is a ribonucleotide residue and the consensus for Gln is:
R₀, R₁₈, R₂₃= are absent
R₁₄, R₂₄, R₅₇= are independently A or absent;
R₁₇, R₇₁= are independently A,C or absent;
R₂₅, R₂₆, R₃₃, R₄₄, R₄₆, R₅₆, R₆₉= are independently A,C,G or absent;
R₄, R₅, R₁₂, R₂₂, R₂₉, R₃₀, R₄₈, R₄₉, R₆₃, R₆₇, R₆₈= are independently N or absent;
R₃₁, R₄₃, R₆₂, R₆₅, R₇₀= are independently A,C,U or absent;
R₁₅, R₂₇, R₃₄, R₄₀, R₄₁, R₅₁, R₅₂= are independently A,G or absent;
R₂, R₇, R₂₁, R₄₅, R₅₀, R₅₈, R₆₆, R₂₂= are independently A,G,U or absent;
R₃, R₁₃, R₃₂, R₃₇, R₄₂, R₆₀, R₆₄= are independently C,G,U or absent;
R₆, R₁₁, R₂₈, R₃₅, R₅₅, R₅₉, R₆₁= are independently C,U or absent;
R₉, R₁₀, R₁₉, R₂₀= are independently G or absent;
R₁, RLO, R₃₉= are independently G,U or absent;
R₈, R_{36, R}_{₃₈}, R₅₃, R₅₄= are independently U or absent;
[R₄₇]ₓ═N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III _{GLN} (SEQ ID NO: 579),

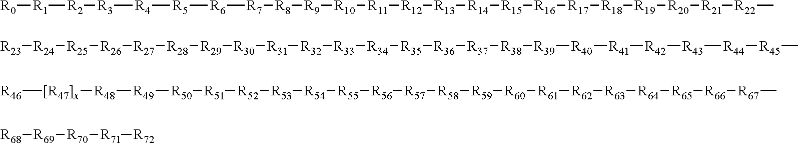

wherein R is a ribonucleotide residue and the consensus for Gln is:
R₀, R₁₈, R₂₃= are absent
R₁₄, R₂₄, R₄₁, R₅₇= are independently A or absent;
R₁₇, R₇₁= are independently A,C or absent;
R₅, R₂₅, R₂₆, R₄₆, R₅₆, R₆₉= are independently A,C,G or absent;
R₄, R₂₂, R₂₉, R₃₀, R₄₈, R₄₉, R₆₃, R₆₈= are independently N or absent;
R₄₃, R₆₂, R₆₅, R₇₀= are independently A,C,U or absent;
R₁₅, R₂₇, R₃₃, R₃₄, R₄₀, R₅₁, R₅₂= are independently A,G or absent;
R₂, R₇, R₁₂, R₄₅, R₅₀, R₅₈, R₆₆= are independently A,G,U or absent;
R₃₁=A,U or absent;
R₃₂, R₄₄, R₆₀= are independently C,G or absent;
R₃, R₁₃, R₃₇, R₄₂, R₆₄, R₆₇= are independently C,G,U or absent;
R₆, R₁₁, R₂₈, R₃₅, R₅₅, R₅₉, R₆₁= are independently C,U or absent;
R₉, R₁₀, R₁₉, R₂₀= are independently G or absent;
R₁, R₂₁, R₃₉, R₂= are independently G,U or absent;
R₈, R₁₆, R₃₆, R₃₈, R₅₃, R₅₄= are independently U or absent;
[R₄₇]ₓ═N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Glutamate TREM Consensus Sequence

In an embodiment, a TREM disclosed herein comprises the sequence of Formula I _{GLU} (SEQ ID NO: 580),

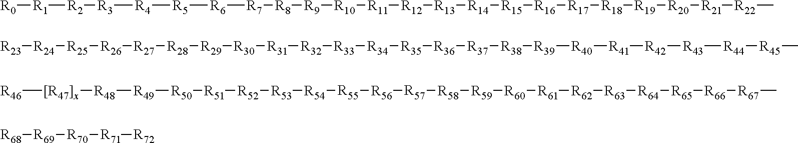

wherein R is a ribonucleotide residue and the consensus for Glu is:
R₀=absent;
R₃₄, R₄₃, R₆₈, R₆₉= are independently A,C,G or absent;
R₁, R₂, R₅, R₆, R₉, R₁₂, R₁₆, R₂₀, R₂₁, R₂₆, R₂₇, R₂₉, R₃₀, R₃₁, R₃₂, R₃₃, R₄₁, R₄₄, R₄₅, R₄₆, R₄₅, R₅₀, R₅₁, R₅₅, R_{6 3}, R₆₄, R₆₅, R₆₆, R₀₀, R₇₁= are independently N or absent;
R₁₃, R₁₇, R₂₃, R₆₁= are independently A,C,U or absent;
R₁₀, R₁₄, R₂₄, R₄₀, R₅₂, R₅₆= are independently A,G or absent;
R₇, R₁₅, R₂₅, R₆₇, R₇₂= are independently A,G,U or absent;
R₁₁, R₅₇= are independently A,U or absent;
R₃₉=C,G or absent;
R₃, R₄, R₂₂, R₄₂, R₄₉, R₅₅, R₆₂= are independently C,G,U or absent;
R₁₈, R₂₈, R₃₅, R₃₇, R₅₃, R₅₉, R₆₀= are independently C,U or absent;
R₁₉=G or absent;
R₈, R₃₆, R₃₈, R₅₄= are independently U or absent;
[R₄₇]ₓ═N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II _{GLU} (SEQ ID NO: 581),

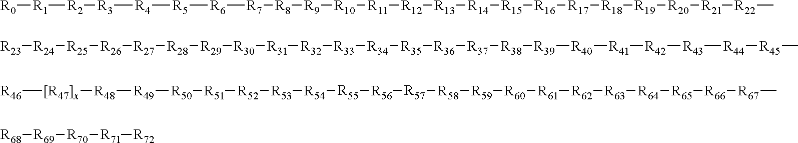

wherein R is a ribonucleotide residue and the consensus for Glu is:
R₀, R₁₅, R₂₃= are absent
R₁₇, R₄₀= are independently A or absent;
R₂₆, R₂₇, R₃₄, R₄₃, R₆₈, R₆₉, R₇₁= are independently A,C,G or absent;
R₁, R₂, R₅, R₁₂, R₂₁, R₃₁, R₃₃, R₄₁, R₄₅, R₄₅, R₅₁, R₅₅, R₆₆, R₇₀= are independently N or absent;
R₄₄, R₆₁= are independently A,C,U or absent;
R₉, R₁₄, R₂₄, R₂₅, R₅₂, R₅₆, R₆₃= are independently A,G or absent;
R₇, R₁₅, R₄₆, R₅₀, R₆₇, R₇₂= are independently A,G,U or absent;
R₂₉, R₅₇= are independently A,U or absent;
R₆₀=C or absent;
R₃₉=C,G or absent;
R₃, R₆, R₂₀, R₃₀, R₃₂, R₄₂, R₅₅, R₆₂, R₆₅= are independently C,G,U or absent;
R₄, R₅, R₁₆, R₂₈, R₃₅, R₃₇, R₄₉, R₅₃, R₅₉= are independently C,U or absent;
R₁₀, R₁₉= are independently G or absent;
R₂₂, R₆₄= are independently G,U or absent;
R₁₁, R₁₃, R₃₆, R₃₈, R₅₄= are independently U or absent;
[R₄₇]ₓ═N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III _{GLU} (SEQ ID NO: 582),

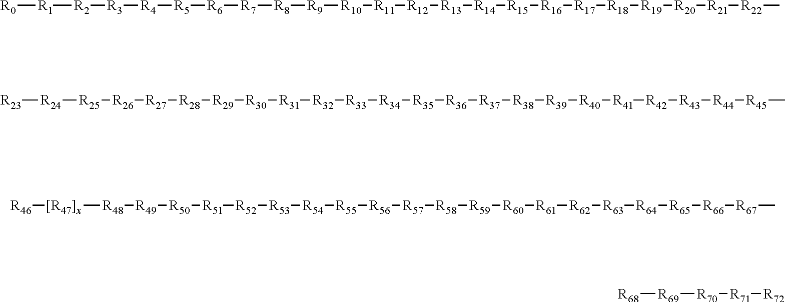

wherein R is a ribonucleotide residue and the consensus for Glu is:
R₀, R₁₇, R₁₅, R₂₃= are absent
R₁₄, R₂₇, R₄₀, R₇₁= are independently A or absent;
R₄₄=A,C or absent;
R₄₃=A,C,G or absent;
R₁, R₃₁, R₃₃, R₄₅, R₅₁, R₆₆= are independently N or absent;
R₂₁, R₄₁= are independently A,C,U or absent;
R₇, R₂₄, R₂₅, R₅₀, R₅₂, R₅₆, R₆₃, R₆₈, R₇₀= are independently A,G or absent;
R₅, R₄₆= are independently A,G,U or absent;
R₂₉, R₅₇, R₆₇, R₂= are independently A,U or absent;
R₂, R₃₉, R₆₀= are independently C or absent;
R₃, R₁₂, R₂₀, R₂₆, R₃₄, R₆₉= are independently C,G or absent;
R₆, R₃₀, R₄₂, R₄₈, R₆₅= are independently C,G,U or absent;
R₄, R₁₆, R₂₅, R₃₅, R₃₇, R₄₉, R₅₃, R₅₅, R₅₈, R₆₁, R₆₂= are independently C,U or absent;
R₉, R₁₀, R₁₉, R₆₄= are independently G or absent;
R₁₅, R₂₂, R₃₂= are independently G,U or absent;
R₈, R₁₁, R₁₃, R₃₆, R₃₈, R₅₄, R₅₉= are independently U or absent;
[R₄₇]ₓ═N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x1-21, x=1-20, x=1-19, x1-18, x1-17, x=1-16, x1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Glycine TREM Consensus Sequence

In an embodiment, a TREM disclosed herein comprises the sequence of Formula I GLY (SEQ ID NO: 583),

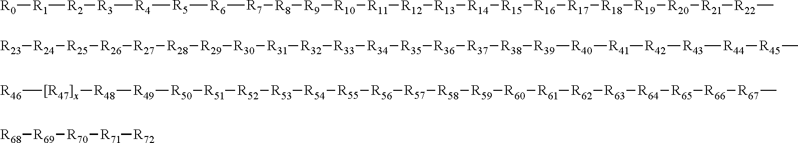

wherein R is a ribonucleotide residue and the consensus for Gly is:
R₀=absent;
R₂₄=A or absent;
R₃, R₉, R₄₀, R₅₀, R₅₁= are independently A,C,G or absent;
R₄, R₅, R₆, R₇, R₂, R₁₆, R₂₁, R₂₂, R₂₆, R₂₉, R₃₀, R₃₁, R₃₂, R₃₃, R₃₄, R₄₁, R₄₂, R₄₃, R₄₄, R₄₅, R₄₆, R₄₅, R₄₉, R₅₈, R₆₃, R₆₄, R₆₅, R₆₆, R₆₇, R₆₈= are independently N or absent;
R₅₉=A,C,U or absent;
R₁, R₁₀, R₁₄, R₁₅, R₂₇, R₅₆= are independently A,G or absent;
R₂₀, R₂₅= are independently A,G,U or absent;
R₅₇, R₇₂= are independently A,U or absent;
R₃₈, R₃₉, R₆₀= are independently C or absent;
R₅₂=C,G or absent;
R₂, R₁₉, R₃₇, R₅₄, R₅₅, R₆₁, R₆₂, R₆₉, R₇₀= are independently C,G,U or absent;
R₁₁, R₁₃, R₁₇, R₂₅, R₃₅, R₃₆, R₇₁= are independently C,U or absent;
R₈, R₁₈, R₂₃, R₅₃= are independently U or absent;
[R₄₇]ₓ═N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II _{GLY}(SEQ ID NO: 584),

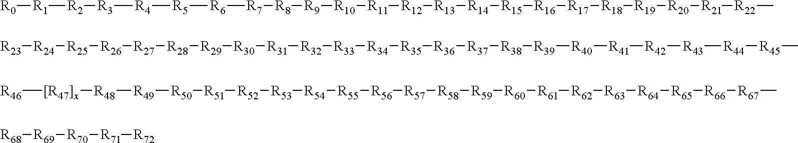

wherein R is a ribonucleotide residue and the consensus for Gly is:
R₀, R₁₈, R₂₃= are absent
R₂₄, R₂₇, R₄₀, R₇₂= are independently A or absent;
R₂₆=A,C or absent;
R₃, R₇, R₆₈= are independently A,C,G or absent;
R₅, R₃₀, R₄₁, R₄₂, R₄₄, R₄₉, R₆₇= are independently A,C,G,U or absent;
R₃₁, R₃₂, R₃₄= are independently A,C,U or absent;
R₉, R₁₀, R₁₄, R₁₅, R₃₃, R₅₀, R₅₆= are independently A,G or absent;
R₁₂, R₁₆, R₂₂, R₂₅, R₂₉, R₄₆= are independently A,G,U or absent;
R₅₇=A,U or absent;
R₁₇, R₃₈, R₃₉, R₆₀, R₆₁, R₇₁= are independently C or absent;
R₆, R₅₂, R₆₄, R₆₆= are independently C,G or absent;
R₂, R₄, R₃₇, R₄₈, R₅₅, R₆₅= are independently C,G,U or absent;
R₁₃, R₃₅, R₄₃, R₆₂, R₆₉= are independently C,U or absent;
R₁, R₁₉, R₂₀, R₅₁, R₇₀= are independently G or absent;
R₂₁, R₄₅, R₆₃= are independently G,U or absent;
R₈, R₁₁, R₂₅, R₃₆, R₅₃, R₅₄, R₅₅, R₅₉= are independently U or absent;
[R₄₇]ₓ═N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III GLY (SEQ ID NO: 585),

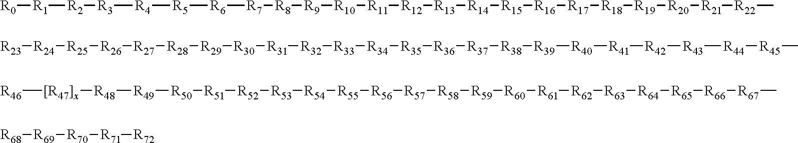

wherein R is a ribonucleotide residue and the consensus for Gly is:
R₀, R₁₈, R₂₃= are absent
R₂₄, R₂₇, R₄₀, R₇₂= are independently A or absent;
R₂₆=A,C or absent;
R₃, R₇, R₄₉, R₆₈= are independently A,C,G or absent;
R₅, R₃₀, R₄₁, R₄₄, R₆₇= are independently N or absent;
R₃₁, R₃₂, R₃₄= are independently A,C,U or absent;
R₉, R₁₀, R₁₄, R₁₅, R₃₃, R₅₀, R₅₆= are independently A,G or absent;
R₁₂, R₂₅, R₂₉, R₄₂, R₄₆= are independently A,G,U or absent;
R₁₀, R₅₇= are independently A,U or absent;
R₁₇, R₃₅, R₃₉, R₆₀, R₆₁, R₇₁= are independently C or absent;
R₆, R₅₂, R₆₄, R₆₆= are independently C,G or absent;
R₃₇, R₄₈, R₆₅= are independently C,G,U or absent;
R₂, R₄, R₁₃, R₃₅, R₄₃, R₅₅, R₆₂, R₆₉= are independently C,U or absent;
R₁, R₁₉, R₂₀, R₅₁, R₇₀= are independently G or absent;
R₂₁, R₂₂, R₄₅, R₆₃= are independently G,U or absent;
R₈, R₁₁, R₂₈, R₃₆, R₅₃, R₅₄, R₅₈, R₅₉= are independently U or absent;
[R₄₇]ₓ═N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Histidine TREM Consensus Sequence

In an embodiment, a TREM disclosed herein comprises the sequence of Formula I HIS (SEQ ID NO: 586),

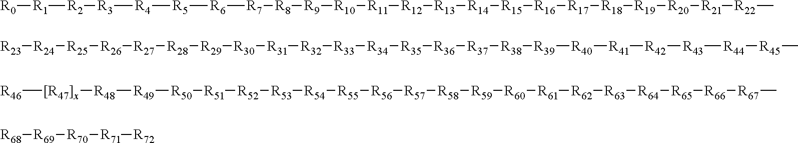

wherein R is a ribonucleotide residue and the consensus for His is:
R₂₃=absent;
R₁₄, R₂₄, R₅₇= are independently A or absent;
R₇₂=A,C or absent;
R₉, R₂₇, R₄₃, R₄₈, R₆₉= are independently A,C,G or absent;
R₃, R₄, R₅, R₆, R₁₂, R₂₅, R₂₆, R₂₉, R₃₀, R₃₁, R₃₄, R₄₂, R₄₅, R₄₆, R₄₉, R₅₀, R₅₈, R₆₂, R₆₃, R₆₆, R₆₇, R₆₈= are independently N or absent;
R₁₃, R₂₁, R₄₁, R₄₄, R₆₅= are independently A,C,U or absent;
R₄₀, R₅₁, R₅₆, R₇₀= are independently A,G or absent;
R₇, R₃₂= are independently A,G,U or absent;
R₅₅, R₀= are independently C or absent;
R₁₁, R₁₆, R₃₃, R₆₄= are independently C,G,U or absent;
R₂, R₁₇, R₂₂, R₂₃, R₃₅, R₅₃, R₅₉, R₆₁, R₇₁= are independently C,U or absent;
R₁, R₁₀, R₁₅, R₁₉, R₂₀, R₃₇, R₃₉, R₅₂= are independently G or absent;
R₀=G,U or absent;
R₈, R₁₅, R₃₆, R₃₈, R₅₄= are independently U or absent;
[R₄₇]ₓ═N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II _{HIS} (SE ID NO: 587),

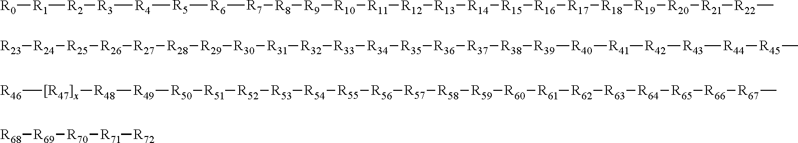

wherein R is a ribonucleotide residue and the consensus for His is:
R₀, R₁₇, R₁₅, R₂₃= are absent;
R₇, R₁₂, R₁₄, R₂₄, R₂₇, R₄₅, R₅₇, R₅₈, R₆₃, R₆₇, R₇₂= are independently A or absent;
R₃=A,C,U or absent;
R₄, R₄₃, R₅₆, R₇₀= are independently A,G or absent;
R₄₉=A,U or absent;
R₂, R₂₈, R₃₀, R₄₁, R₄₂, R₄₄, R₄₈, R₅₅, R₆₀, R₆₆, R₇₁= are independently C or absent;
R₂₅=C,G or absent;
R₉=C,G,U or absent;
R₈, R₁₃, R₂₆, R₃₃, R₃₅, R₅₀, R₅₃, R₆₁, R₆₈= are independently C,U or absent;
R₁, R₆, R₁₀, R₅, R₁₉, R₂, R₃₂, R₃₄, R₃₇, R₃₉, R₄, R₄₆, R₅₁, R₅₂, R₆₂, R₆₄, R₆₉= are independently G or absent;
R₁₆=G,U or absent;
R₅, R₁₁, R₂₁, R₂₂, R₂₉, R₃₁, R₃₆, R₃₈, R₅₄, R₅₉, R₆₅= are independently U or absent;
[R₄₇]ₓ═N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III HIS (SEQ ID NO: 588),

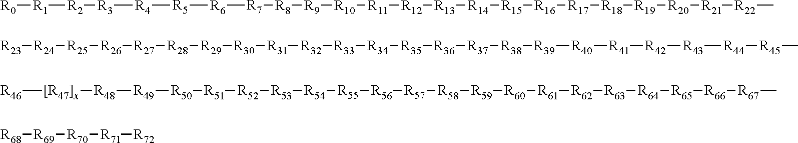

wherein R is a ribonucleotide residue and the consensus for His is:
R₀, R₁₇, R₁₅, R₂₃= are absent
R₇, R₁₂, R₁₄, R₂₄, R₂₇, R₄₅, R₅₇, R₅₈, R₆₃, R₆₇, R₂= are independently A or absent;
R₃=A,C or absent;
R₄, R₄₃, R₅₆, R₇₀= are independently A,G or absent;
R₄₉=A,U or absent;
R₂, R₂₈, R₃₀, R₄₁, R₄₂, R₄₄, R₄₈, R₅₅, R₆₀, R₆₆, R₇₁= are independently C or absent;
R₈, R₉, R₂₆, R₃₃, R₃₅, R₅₀, R₆₁, R₆₈= are independently C,U or absent;
R₁, R₆, R₁₀, R₁₅, R₁₉, R₂₀, R₂₅, R₃₂, R₃₄, R₃₇, R₃₉, R₄₀, R₄₆, R₅₁, R₅₂, R₆₂, R₆₄, R₆₉= are independently G or absent;
R₅, R₁₁, R₁₃, R₁₆, R₂₁, R₂₂, R₂₉, R₃₁, R₃₆, R₃₈, R₅₃, R₅₄, R₅₉, R₆₅= are independently U or absent;
[R₄₇]ₓ═N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Isoleucine TREM Consensus Sequence

In an embodiment, a TREM disclosed herein comprises the sequence of Formula I I_{LE} (SEQ ID NO: 589),

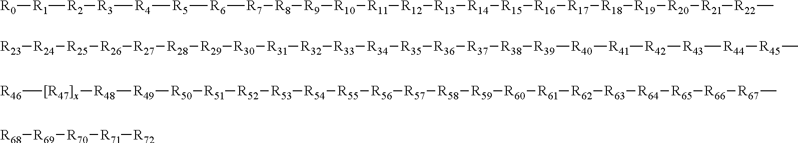

wherein R is a ribonucleotide residue and the consensus for Ile is:
R₂₃=absent;
R₃₈, R₄₁, R₅₇, R₇₂= are independently A or absent;
R₁, R₂₆= are independently A,C,G or absent;
R₀, R₃, R₄, R₆, R₁₆, R₃₁, R₃₂, R₃₄, R₃₇, R₄₂, R₄₃, R₄₄, R₄₅, R₄₆, R₄₈, R₄₉, R₅₀, R₅₈, R₅₉, R₆₂, R₆₃, R₆₄, R₆₆, R₆₇, R₆₈, R₆₉= are independently N or absent;
R₂₂, R₆₁, R₆₅= are independently A,C,U or absent;
R₉, R₁₄, R₁₅, R₂₄, R₂₇, R₄₀= are independently A,G or absent;
R₇, R₂₅, R₂₉, R₅₁, R₅₆= are independently A,G,U or absent;
R₁₅, R₅₄= are independently A,U or absent;
R₆₀═C or absent;
R₂, R₅₂, R₇₀= are independently C,G or absent;
R₅, R₁₂, R₂₁, R₃₀, R₃₃, R₇₁= are independently C,G,U or absent;
R₁₁, R₁₃, R₁₇, R₂₈, R₃₅, R₅₃, R₅₅= are independently C,U or absent;
R₁₀, R₁₉, R₂₀= are independently G or absent;
R₈, R₃₆, R₃₉= are independently U or absent;
[R₄₇]ₓ═N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II _{ILE} (SEQ ID NO: 590),

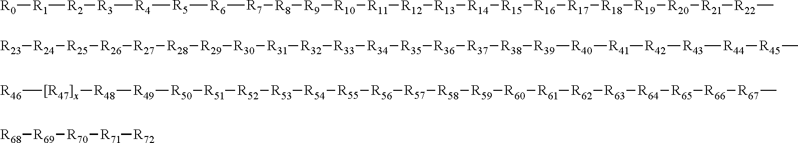

wherein R is a ribonucleotide residue and the consensus for Ile is:
R₀, R₁₈, R₂₃= are absent
R₂₄, R₃₈, R₄₀, R₄₁, R₅₇, R₇₂= are independently A or absent;
R₂₆, R₆₅= are independently A,C or absent;
R₅₈, R₅₉, R₆₇= are independently N or absent;
R₂₂=A,C,U or absent;
R₆, R₉, R₁₄, R₁₅, R₂₉, R₃₄, R₄₃, R₄₆, R₄₈, R₅₀, R₅₁, R₆₃, R₆₉= are independently A,G or absent;
R₃₇, R₅₆= are independently A,G,U or absent;
R₅₄=A,U or absent;
R₂₈, R₃₅, R₆₀, R₆₂, R₇₁= are independently C or absent;
R₂, R₅₂, R₇₀= are independently C,G or absent;
R₅=C,G,U or absent;
R₃, R₄, R₁₁, R₁₃, R₁₇, R₂₁, R₃₀, R₄₂, R₄₄, R₄₅, R₄₉, R₅₃, R₅₅, R₆₁, R₆₄, R₆₆= are independently C,U or absent;
R₁, R₁₀, R₁₉, R₂₀, R₂₅, R₂₇, R₃₁, R₆₈= are independently G or absent;
R₇, R₁₂, R₃₂= are independently G,U or absent;
R₈, R₁₆, R₃₃, R₃₆, R₃₉= are independently U or absent;
[R₄₇]ₓ═N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III ILE (SEQ ID NO: 591),

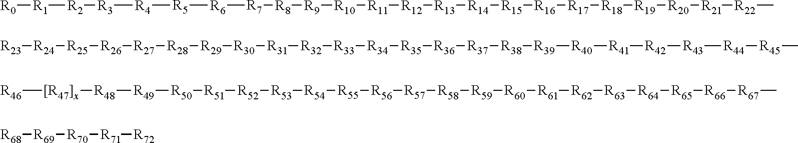

wherein R is a ribonucleotide residue and the consensus for Ile is:
R₀, R₁₁, R₂₃= are absent
R₁₄, R₂₄, R₃₈, R₄₀, R₄₁, R₅₇, R₇₂= are independently A or absent;
R₂₆, R₆₅= are independently A,C or absent;
R₂₂, R₅₉= are independently A,C,U or absent;
R₆, R₉, R₁₅, R₃₄, R₄₃, R₄₆, R₅₁, R₅₆, R₆₃, R₆₉= are independently A,G or absent;
R₃₇=A,G,U or absent;
R₁₃, R₂₈, R₃₅, R₄₄, R₅₅, R₆₀, R₆₂, R₇₁= are independently C or absent;
R₂, R₅, R₇₀= are independently C,G or absent;
R₅₈, R₆₇= are independently C,G,U or absent;
R₃, R₄, R₁₁, R₁₇, R₂₁, R₃₀, R₄₂, R₄₅, R₄₉, R₅₃, R₆₁, R₆₄, R₆₆= are independently C,U or absent;
R₁, R₁₀, R₁₉, R₂₀, R₂, R₇, R₂₉, R₃₁, R₃₂, R₄₈, R₅₀, R₅₂, R₆₈= are independently G or absent;
R₇, R₁₂= are independently G,U or absent;
R₈, R₁₆, R₃₃, R₃₆, R₃₉, R₅₄= are independently U or absent;
[R₄₇]ₓ═N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Methionine TREM Consensus Sequence

In an embodiment, a TREM disclosed herein comprises the sequence of Formula I _{MET} (SEQ ID NO: 592),

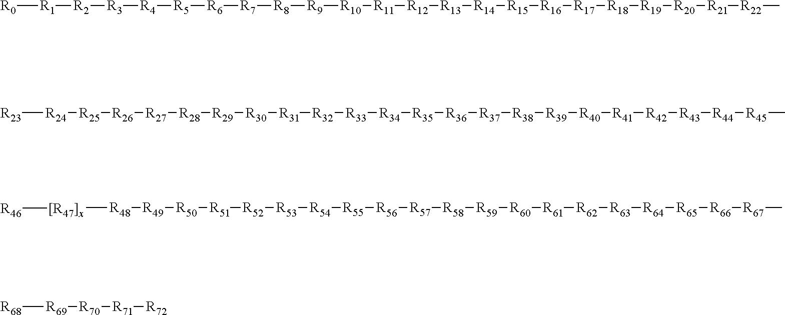

wherein R is a ribonucleotide residue and the consensus for Met is:
R₀, R₂₃= are absent;
R₁₄, R₃₈, R₄₀, R₅₇= are independently A or absent;
R₆₀=A,C or absent;
R₃₃, R₄₈, R₇₀= are independently A,C,G or absent;
R₁, R₃, R₄, R₅, R₆, R₁₁, R₁₂, R₁₆, R₁₇, R₂₁, R₂₂, R₂₆, R₂₇, R₂₉, R₃₀, R₃₁, R₃₂, R₄₂, R₄₄, R₄₅, R₄₆, R₄₉, R₅₀, R₅₈, R₆ 2, R₆₃, R₆₆, R₆₇, R₆₈, R₆₉, R₇₁= are independently N or absent;
R₁₈, R₃₅, R₄₁, R₅₉, R₆₅= are independently A,C,U or absent;
R₉, R₁₅, R₅₁= are independently A,G or absent;
R₇, R₂₄, R₂₅, R₃₄, R₅₃, R₅₆= are independently A,G,U or absent;
R₇₂=A,U or absent;
R₃₇=C or absent;
R₁₀, R₅₅= are independently C,G or absent;
R₂, R₁₃, R₂₈, R₄₃, R₆₄= are independently C,G,U or absent;
R₃₆, R₆₁= are independently C,U or absent;
R₁₉, R₂₀, R₅₂= are independently G or absent;
R₈, R₃₉, R₅₄= are independently U or absent;
[R₄₇]ₓ ═N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II _{MET}(SEQ ID NO: 593),

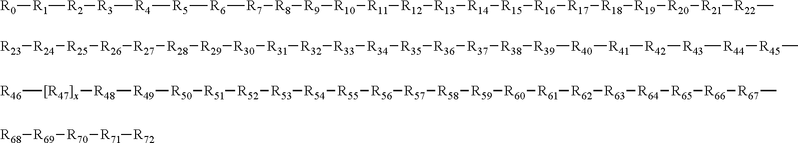

wherein R is a ribonucleotide residue and the consensus for Met is:
R₀, R₁₈, R₂₂, R₂₃= are absent
R₁₄, R₂₄, R₃₈, R₄₀, R₄₁, R₅₇, R₇₂= are independently A or absent;
R₅₉, R₆₀, R₆₂, R₆₅= are independently A,C or absent;
R₆, R₄₅, R₆₇= are independently A,C,G or absent;
R₄=N or absent;
R₂1, R₄₂= are independently A,C,U or absent;
R₁, R₉, R₂₇, R₂₉, R₃₂, R₄₆, R₅₁= are independently A,G or absent;
R₁₇, R₄₉, R₅₃, R₅₆, R₅₈= are independently A,G,U or absent;
R₆₃=A,U or absent;
R₃, R₁₃, R₃₇= are independently C or absent;
R₄₈, R₅₅, R₆₄, R₇₀= are independently C,G or absent;
R₂, R₅, R₆₆, R₆₈= are independently C,G,U or absent;
R₁₁, R₁₆, R₂₆, R₂₈, R₃₀, R₃₁, R₃₅, R₃₆, R₄₃, R₄₄, R₆₁, R₇₁= are independently C,U or absent;
R₁₀, R₁₂, R₁₅, R₁₉, R₂₀, R₂₅, R₃₃, R₅₂, R₆₉= are independently G or absent;
R₇, R₃₄, R₅₀= are independently G,U or absent;
R₈, R₃₉, R₅₄= are independently U or absent;
[R₄₇]ₓ═N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III _{MET} (SEQ ID NO: 594),

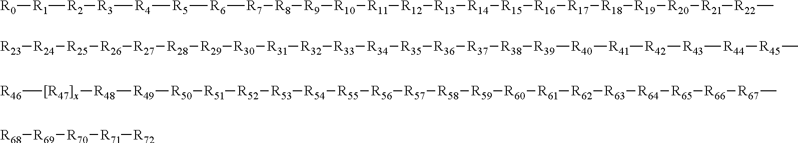

wherein R is a ribonucleotide residue and the consensus for Met is:
R₀, R₁₈, R₂₂, R₂₃= are absent
R₁₄, R₂₄, R₃₈, R₄₀, R₄₁, R₅₇, R₂₂= are independently A or absent;
R₅₉, R₆₂, R₆₅= are independently A,C or absent;
R₆, R₆₇= are independently A,C,G or absent;
R₄, R₂₁= are independently A,C,U or absent;
R₁, R₉, R₂₇, R₂₉, R₃₂, R₄₅, R₄₆, R₅₁= are independently A,G or absent;
R₁₇, R₅₆, R₅₈= are independently A,G,U or absent;
R₄₉, R₅₃, R₆₃= are independently A,U or absent;
R₃, R₁₃, R₂₆, R₃₇, R₄₃, R₆₀= are independently C or absent;
R₂, R₄₈, R₅₅, R₆₄, R₇₀= are independently C,G or absent;
R₅, R₆₆= are independently C,G,U or absent;
R₁₁, R₁₆, R₂₈, R₃₀, R₃₁, R₃₅, R₃₆, R₄₂, R₄₄, R₆₁, R₇₁= are independently C,U or absent;
R₁₀, R₁₂, R₁₅, R₁₉, R₂₀, R₂₅, R₃₃, R₅₂, R₆₉= are independently G or absent;
R₇, R₃₄, R₅₀, R₆₈= are independently G,U or absent;
R₈, R₃₉, R₅₄= are independently U or absent;
[R₄₇]ₓ═N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Leucine TREM Consensus Sequence

In an embodiment, a TREM disclosed herein comprises the sequence of Formula I _{LEU} (SEQ ID NO: 595),

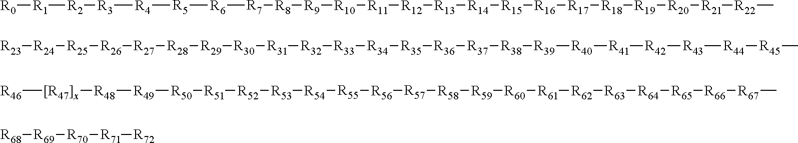

wherein R is a ribonucleotide residue and the consensus for Leu is:
R₀=absent;
R₃₈, R₅₇= are independently A or absent;
R₆₀=A,C or absent;
R₁, R₁₃, R₂₇, R₄₈, R₅₁, R₅₆= are independently A,C,G or absent;
R₂, R₃, R₄, R₅, R₆, R₇, R₉, R₁₀, R₁₁, R₂, R₁₆, R₂₃, R₂₆, R₂₈, R₂₉, R₃₀, R₃₁, R₃₂, R₃₃, R₃₄, R₃₇, R₄₁, R₄₂, R₄₃, R₄₄, R₄₅, R₄₆, R₄₉, R₅₀, R₅₈, R₆₂, R₆₃, R₆₅, R₆₆, R₆₇, R₆₈, R₆₉, R₇₀= are independently N or absent;
R₁₇, R₁₅, R₂₁, R₂₂, R₂₅, R₃₅, R₅₅= are independently A,C,U or absent;
R₁₄, R₁₅, R₃₉, R₇₂= are independently A,G or absent;
R₂₄, R₄₀= are independently A,G,U or absent;
R₅₂, R₆₁, R₆₄, R₇₁= are independently C,G,U or absent;
R₃₆, R₅₃, R₅₉= are independently C,U or absent;
R₁₉=G or absent;
R₂₀=G,U or absent;
R₈, R₅₄=are independently U or absent;
[R₄₇]ₓ═N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II _{LEU} (SEQ ID NO: 596),

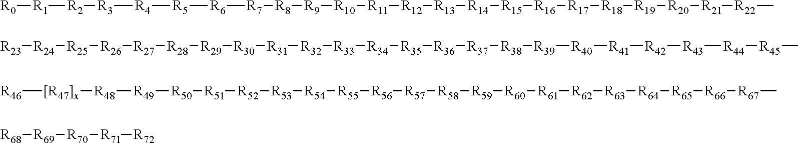

wherein R is a ribonucleotide residue and the consensus for Leu is:
R₀=absent
R₃₅, R₅₇, R₇₂=are independently A or absent;
R₆₀=A, C or absent;
R₄, R₅, R₄₈, R₅₀, R₅₆, R₆₉=are independently A, C, G or absent;
R₆, R₃₃, R₄₁, R₄₃, R₄₆, R₄₉, R₅₈, R₆₃, R₆₆, R₇₀=are independently N or absent;
R₁₁, R₁₂, R₁₇, R₂₁, R₂₂, R₂₈, R₃₁, R₃₇, R₄₄, R₅₅=are independently A, C, U or absent;
R₁, R₉, R₁₄, R₁₅, R₂₄, R₂₇, R₃₄, R₃₉=are independently A, G or absent;
R₇, R₂₉, R₃₂, R₄₀, R₄₅=are independently A, G, U or absent;
R₂₅=A, U or absent;
R₁₃=C, G or absent;
R₂, R₃, R₁₆, R₂₆, R₃₀, R₅₂, R₆₂, R₆₄, R₆₅, R₆₇, R₆₈=are independently C, G, U or absent;
R₁₈, R₃₅, R₄₂, R₅₃, R₅₉, R₆₁, R₇₁=are independently C, U or absent;
R₁₉, R₅₁=are independently G or absent; R₁₀, R₂₀=are independently G, U or absent;
R₈, R₂₃, R₃₆, R₅₄=are independently U or absent;
[R₄₇]ₓ═N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III LEU (SEQ ID NO: 597),

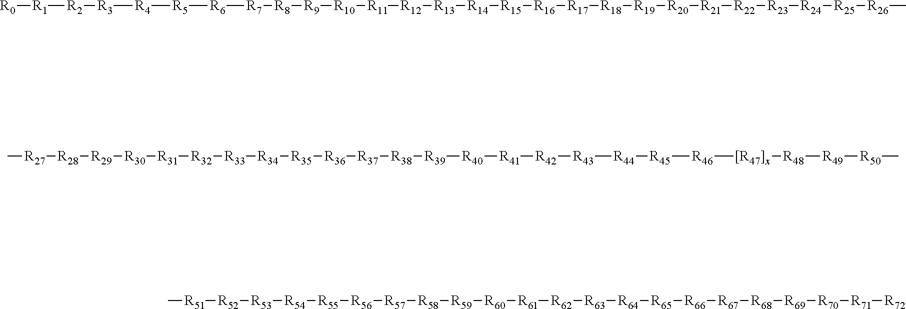

wherein R is a ribonucleotide residue and the consensus for Leu is:
R₀=absent
R₃₈, R₅₇, R₇₂=are independently A or absent;
R₆₀=A, C or absent;
R₄, R₅, R₄₈, R₅₀, R₅₆, R₅₈, R₆₉=are independently A, C, G or absent;
R₆, R₃₃, R₄₃, R₄₆, R₄₉, R₆₃, R₆₆, R₇₀=are independently N or absent;
R₁₁, R₁₂, R₁₇, R₂₁, R₂₂, R₈, R₃₁, R₃₇, R₄₁, R₄₄, R₅₅=are independently A, C, U or absent;
R₁, R₉, R₁₄, R₁₅, R₂₄, R₂₇, R₃₄, R₃₉=are independently A, G or absent;
R₇, R₂₉, R₃₂, R₄₀, R₄₅=are independently A, G, U or absent;
R₂₅=A, U or absent;
R₁₃=C, G or absent;
R₂, R₃, R₁₆, R₃₀, R₅₂, R₆₂, R₆₄, R₆₇, R₆₈=are independently C, G, U or absent;
R₁₈, R₃₅, R₄₂, R₅₃, R₅₉, R₆₁, R₆₅, R₇₁=are independently C, U or absent;
R₁₉, R₅₁=are independently G or absent; R₁₀, R₂₀, R₂₆=are independently G, U or absent;
R₈, R₂₃, R₃₆, R₅₄=are independently U or absent;
[R₄₇]ₓ═N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Lysine TREM Consensus Sequence

In an embodiment, a TREM disclosed herein comprises the sequence of Formula I LYS (SEQ ID NO: 598),

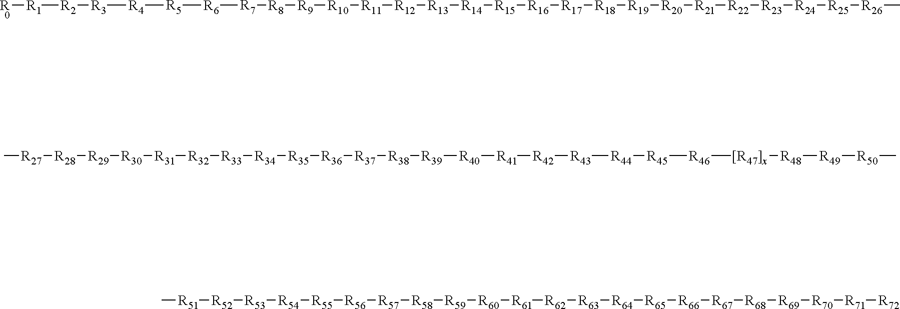

wherein R is a ribonucleotide residue and the consensus for Lys is:
R₀=absent
R₁₄=A or absent;
R₄₀, R₄₁=are independently A, C or absent;
R₃₄, R₄₃, R₅₁=are independently A, C, G or absent;
R₁, R₂, R₃, R₄, R₅, R₆, R₇, R₁₁, R₁₂, R₁₆, R₂₁, R₂₆, R₃₀, R₃₁, R₃₂, R₄₄, R₄₅, R₄₆, R₄₈, R₄₉, R₅₀, R₅₈, R₆₂, R₆₃, R₆₅, R₆₆, R₆₇, R₆₈, R₆₉, R₇₀=are independently N or absent;
R₁₃, R₁₇, R₅₉, R₇₁=are independently A, C, U or absent;
R₉, R₁₅, R₁₉, R₂₀, R₂₅, R₂₇, R₅₂, R₅₆=are independently A, G or absent;
R₂₄, R₂₉, R₇₂=are independently A, G, U or absent;
R₁₈, R₅₇=are independently A, U or absent;
R₁₀, R₃₃=are independently C, G or absent;
R₄₂, R₆₁, R₆₄=are independently C, G, U or absent;
R₂₈, R₃₅, R₃₆, R₃₇, R₅₃, R₅₅, R₆₀=are independently C, U or absent;
R₈, R₂₂, R₂₃, R₃₈, R₃₉, R₅₄=are independently U or absent;
[R₄₇]ₓ═N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II _{LYS}(SEQ ID NO: 599),

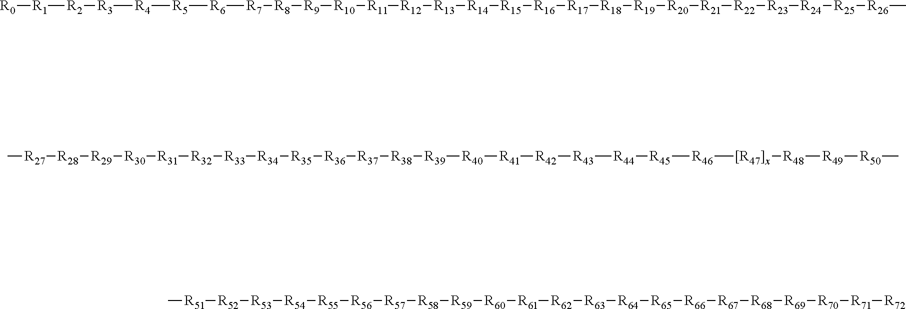

wherein R is a ribonucleotide residue and the consensus for Lys is:
R₀, R₁₈, R₂₃=are absent
R₁₄=A or absent;
R₄₀, R₄₁, R₄₃=are independently A, C or absent;
R₃, R₇=are independently A, C, G or absent;
R₁, R₆, R₁, R₃₁, R₄₅, R₄₈, R₄₉, R₆₃, R₆₅, R₆₆, R₆₈=are independently N or absent;
R₂, R₁₂, R₁₃, R₁₇, R₄₄, R₆₇, R₇₁=are independently A, C, U or absent;
R₉, R₁₅, R₁₉, R₂₀, R₂₅, R₂₇, R₃₄, R₅₀, R₅₂, R₅₆, R₇₀, R₇₂=are independently A, G or absent;
R₅, R₂₄, R₂₆, R₂₉, R₃₂, R₄₆, R₆₉=are independently A, G, U or absent;
R₅₇=A, U or absent;
R₁₀, R₆₁=are independently C, G or absent;
R₄, R₁₆, R₂1, R₃₀, R₅₅, R₆₄=are independently C, G, U or absent;
R₂₈, R₃₅, R₃₆, R₃₇, R₄₂, R₅₃, R₅₅, R₅₉, R₆₀, R₆₂=are independently C, U or absent;
R₃₃, R₅₁=are independently G or absent;
R₈=G, U or absent;
R₂₂, R₃₈, R₃₉, R₅₄=are independently U or absent;
[R₄₇]ₓ═N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III _{LYS} (SEQ ID NO: 600),

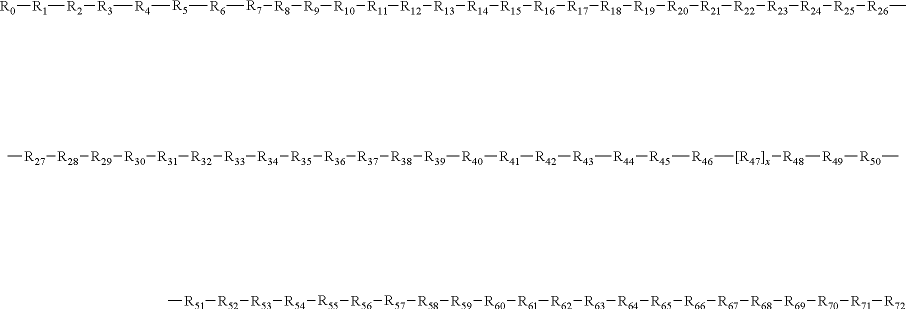

wherein R is a ribonucleotide residue and the consensus for Lys is:
R₀, R₁₈, R₂₃=absent
R₉, R₁₄, R₃₄, R₄₁=are independently A or absent;
R₄₀=A, C or absent;
R₁, R₃, R₇, R₃₁=are independently A, C, G or absent;
R₄₈, R₆₅, R₆₈=are independently N or absent;
R₂, R₁₃, R₁₇, R₄₄, R₆₃, R₆₆=are independently A, C, U or absent;
R₅, R₁₅, R₁₉, R₂, R₂₅, R₂₇, R₂₉, R₅₀, R₅₂, R₅₆, R₀, R₇₂=are independently A, G or absent;
R₆, R₂₄, R₃₂, R₄₉=are independently A, G, U or absent;
R₁₂, R₂₆, R₄₆, R₅₇=are independently A, U or absent;
R₁1, R₂₈, R₃₅, R₄₃=are independently C or absent;
R₁₀, R₄₅, R₆₁=are independently C, G or absent;
R₄, R₂₁, R₆₄=are independently C, G, U or absent;
R₃₇, R₅₃, R₅₅, R₅₉, R₆₀, R₆₂, R₆₇, R₇₁=are independently C, U or absent;
R₃₃, R₅₁=are independently G or absent;
R₈, R₃₀, R₅₈, R₆₉=are independently G, U or absent;
R₁6, R₂₂, R₃₆, R₃₈, R₃₉, R₄₂, R₅₄=are independently U or absent;
[R₄₇]ₓ═N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Phenylalanine TREM Consensus Sequence

In an embodiment, a TREM disclosed herein comprises the sequence of Formula I PHE (SEQ ID NO: 601),

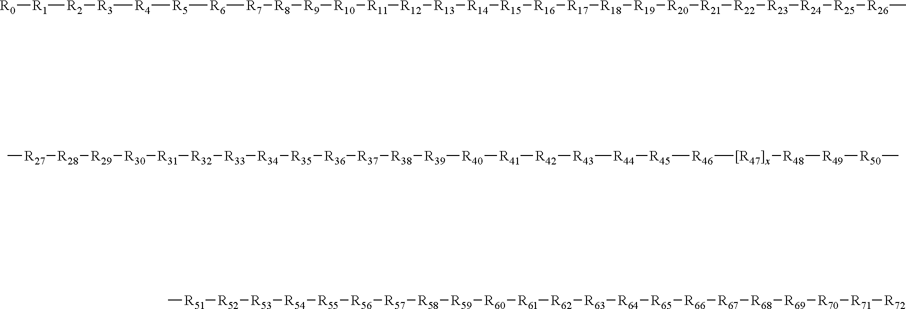

wherein R is a ribonucleotide residue and the consensus for Phe is:
R₀, R₂₃=are absent
R₉, R₁₄, R₃₈, R₃₉, R₅₇, R₇₂=are independently A or absent;
R₇₁=A, C or absent;
R₄₁, R₇₀=are independently A, C, G or absent;
R₄, R₅, R₆, R₃₀, R₃₁, R₃₂, R₃₄, R₄₂, R₄₄, R₄₅, R₄₆, R₄₈, R₄₉, R₅₅, R₆₂, R₆₃, R₆₆, R₆₇, R₆₅, R₆₉=are independently N or absent;
R₁₆, R₆₁, R₆₅=are independently A, C, U or absent;
R₁₅, R₂₆, R₂₇, R₂₉, R₄₀, R₅₆=are independently A, G or absent;
R₇, R₅₁=are independently A, G, U or absent;
R₂₂, R₂₄=are independently A, U or absent;
R₅₅, R₆₀=are independently C or absent;
R₂, R₃, R₂₁, R₃₃, R₄₃, R₅₀, R₆₄=are independently C, G, U or absent;
R₁₁, R₁₂, R₁₃, R₁₇, R₂₈, R₃₅, R₃₆, R₅₉=are independently C, U or absent;
R₁₀, R₁₉, R₂₀, R₂₅, R₃₇, R₅₂=are independently G or absent;
R₁=G, U or absent;
R₈, R₁₈, R₅₃, R₅₄=are independently U or absent;
[R₄₇]ₓ═N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II _{PIIE} (SEQ ID NO: 602),

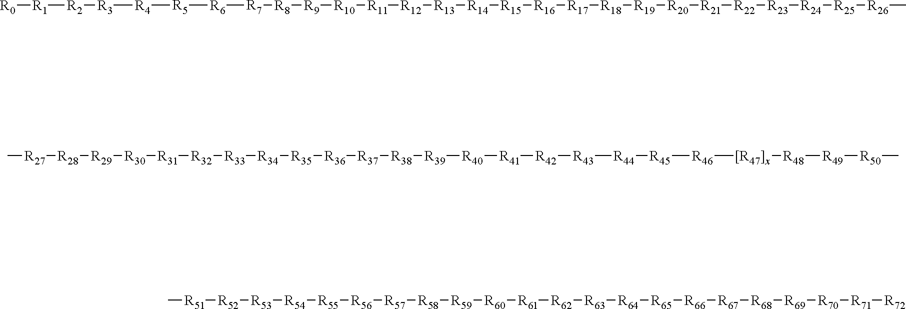

wherein R is a ribonucleotide residue and the consensus for Phe is:
R₀, R₁₈, R₂₃=absent
R₁₄, R₂₄, R₃₈, R₃₉, R₅₇, R₂=are independently A or absent;
R₄₆, R₇₁=are independently A, C or absent;
R₄, R₇₀=are independently A, C, G or absent;
R₄₅=A, C, U or absent;
R₆, R₇, R₁₅, R₂₆, R₂₇, R₃₂, R₃₄, R₄₀, R₄₁, R₅₆, R₆₉=are independently A, G or absent;
R₂₉=A, G, U or absent;
R₅, R₉, R₆₇=are independently A, U or absent;
R₃₅, R₄₉, R₅₅, R₆₀=are independently C or absent;
R₂₁, R₄₃, R₆₂=are independently C, G or absent;
R₂, R₃₃, R₆₈=are independently C, G, U or absent;
R₃, R₁₁, R₁₂, R₁₃, R₂₈, R₃₀, R₃₆, R₄₂, R₄₄, R₄₈, R₅₈, R₅₉, R₆₁, R₆₆=are independently C, U or absent;
R₁₀, R₁₉, R₂₀, R₂₅, R₃₇, R₅₁, R₅₂, R₆₃, R₆₄=are independently G or absent;
R₁, R₃₁, R₅₀=are independently G, U or absent;
R₈, R₁₆, R₁₇, R₂₂, R₅₃, R₅₄, R₆₅=are independently U or absent;
[R₄₇]ₓ═N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III PHE (SEQ ID NO: 603),

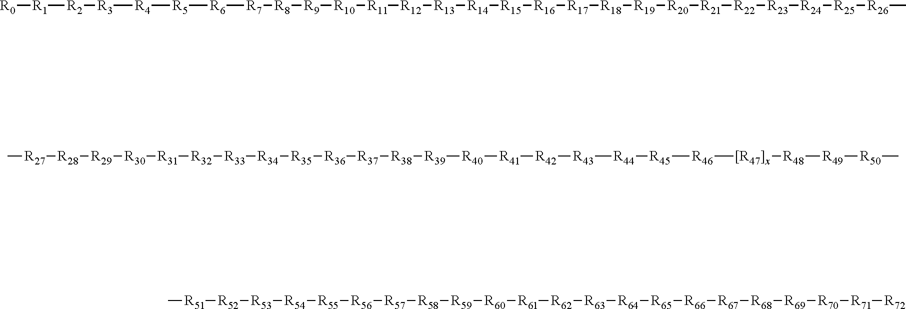

wherein R is a ribonucleotide residue and the consensus for Phe is:
R₀, R₁₅, R₂₂, R₂₃=absent
R₅, R₇, R₁₄, R₂₄, R₂₆, R₃₂, R₃₄, R₃₈, R₃₉, R₄₁, R₅₇, R₇₂=are independently A or absent;
R₄₆=A, C or absent;
R₇₀=A, C, G or absent;
R₄, R₆, R₁5, R₅₆, R₆₉=are independently A, G or absent;
R₉, R₄₅=are independently A, U or absent;
R₂, R₁1, R₁₃, R₃₅, R₄₃, R₄₉, R₅₅, R₆₀, R₆₅, R₇₁=are independently C or absent;
R₃₃=C, G or absent;
R₃, R₂₅, R₃₆, R₄₃, R₅₅, R₅₉, R₆₁=are independently C, U or absent;
R₁, R₁₀, R₁₉, R₂₀, R₂₁, R₂₅, R₂₇, R₂₉, R₃₇, R₄₀, R₅₁, R₅₂, R₆₂, R₆₃, R₆₄=are independently G or absent;
R₈, R₁₂, R₁₆, R₁₇, R₃₀, R₃₁, R₄₂, R₄₄, R₅₀, R₅₃, R₅₄, R₆₅, R₆₆, R₆₇=are independently U or absent;
[R₄₇]ₓ═N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Proline TREM Consensus Sequence

In an embodiment, a TREM disclosed herein comprises the sequence of Formula I _{PRO} (SEQ ID NO: 604),

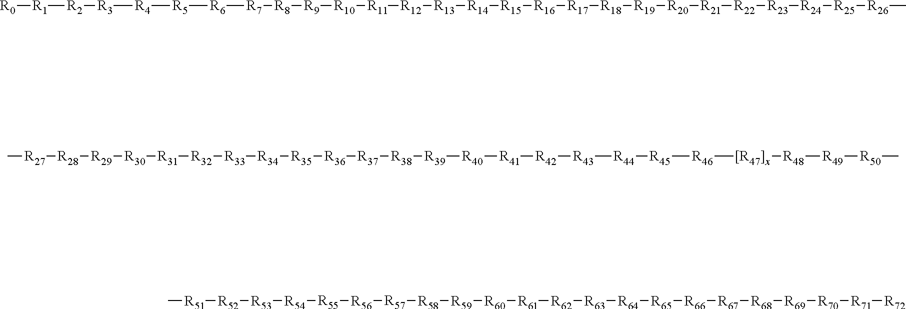

wherein R is a ribonucleotide residue and the consensus for Pro is:
R₀=absent
R₁₄, R₅₇=are independently A or absent;
R₇₀, R₇₂=are independently A, C or absent;
R₉, R₂₆, R₂₇=are independently A, C, G or absent;
R₄, R₅, R₆, R₁₆, R₂₁, R₂₉, R₃₀, R₃₁, R₃₂, R₃₃, R₃₄, R₃₇, R₄₁, R₄₂, R₄₃, R₄₄, R₄₅, R₄₆, R₄₅, R₄₉, R₅₀, R₅₅, R₆₁, R₆₂, R₆₃, R₆₄, R₆₆, R₆₇, R₆₈=are independently N or absent;
R₃₅, R₆₅=are independently A, C, U or absent;
R₂₄, R₄₀, R₅₆=are independently A, G or absent;
R₇, R₂₅, R₅₁=are independently A, G, U or absent;
R₅₅, R₆₀=are independently C or absent;
R₁, R₃, R₇₁=are independently C, G or absent;
R₁, R₁₂, R₂₀, R₆₉=are independently C, G, U or absent;
R₁₃, R₁₇, R₁₈, R₂₂, R₂₃, R₂₈, R₅₉=are independently C, U or absent;
R₁₀, R₁₅, R₁₉, R₃₅, R₃₉, R₅₂=are independently G or absent;
R₂=are independently G, U or absent;
R₈, R₃₆, R₅₃, R₅₄=are independently U or absent;
[R₄₇]ₓ═N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II PRO (SEQ ID NO: 605),

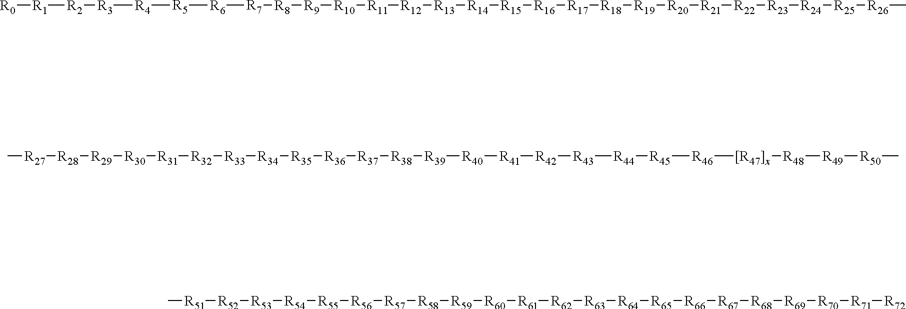

wherein R is a ribonucleotide residue and the consensus for Pro is:
R₀, R₁₇, R₁₅, R₂₂, R₂₃=absent;
R₁₄, R₄₅, R₅₆, R₅₇, R₅₈, R₆₅, R₆₈=are independently A or absent;
R₆₁=A, C, G or absent;
R₄₃=N or absent;
R₃₇=A, C, U or absent;
R₂₄, R₂₇, R₃₃, R₄₀, R₄₄, R₆₃=are independently A, G or absent;
R₃, R₁₂, R₃₀, R₃₂, R₄₈, R₅₅, R₆₀, R₇₀, R₇₁, R₇₂=are independently C or absent;
R₈, R₃₄, R₄₂, R₆₆=are independently C, G or absent;
R₂₀=C, G, U or absent;
R₃₅, R₄1, R₄9, R₆₂=are independently C, U or absent;
R₁, R₂, R₆, R₉, R₁₀, R₁₅, R₁₉, R₂₆, R₃₅, R₃₉, R₄₆, R₅₀, R₅₁, R₅₂, R₆₄, R₆₇, R₆₉=are independently G or absent;
R₁₁, R₁₆=are independently G, U or absent;
R₄, R₇, R₈, R₁₃, R₂₁, R₂₅, R₂₈, R₂₉, R₃1, R₃₆, R₅₃, R₅₄, R₅₉=are independently U or absent;
[R₄₇]ₓ═N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III PRO (SEQ ID NO: 606),

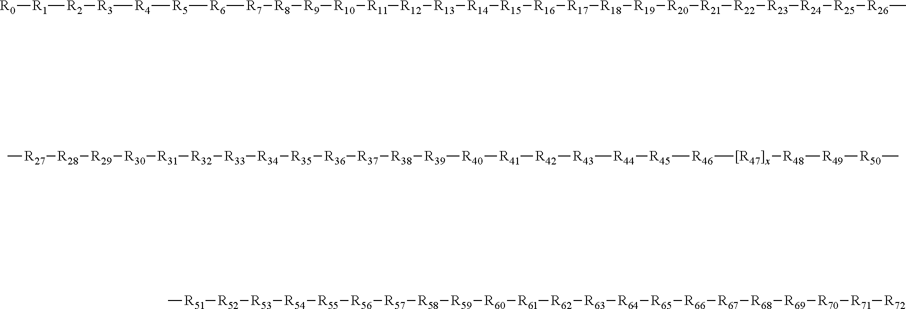

wherein R is a ribonucleotide residue and the consensus for Pro is:
R₀, R₁₇, R₁₈, R₂₂, R₂₃=absent
R₁₄, R₄₅, R₅₆, R₅₇, R₅₈, R₆₅, R₆₈=are independently A or absent;
R₃₇=A, C, U or absent;
R₂₄, R₂₇, R₄₀=are independently A, G or absent;
R₃, R₅, R₁₂, R₃₀, R₃₂, R₄₈, R₄₉, R₅₅, R₆₀, R₆₁, R₆₂, R₆₆, R₇₀, R₇₁, R₇₂=are independently C or absent;
R₃₄, R₄₂=are independently C, G or absent;
R₄₃=C, G, U or absent;
R₄₁=C, U or absent;
R₁, R₂, R₆, R₉, R₁₀, R₁₅, R₉, R₂₀, R₂₆, R₃₃, R₃₅, R₃₉, R₄₄, R₄₆, R₅₀, R₅₁, R₅₂, R₆₃, R₆₄, R₆₇, R₆₉=are independently G or absent;
R₁₆=G, U or absent;
R₄, R₇, R₈, R₁₁, R₁₃, R₂₁, R₂₅, R₂₈, R₂₉, R₃1, R₃₅, R₃₆, R₅₃, R₅₄, R₅₉=are independently U or absent;
[R₄₇]ₓ═N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Serine TREM Consensus Sequence

In an embodiment, a TREM disclosed herein comprises the sequence of Formula I _{SER} (SEQ ID NO: 607),

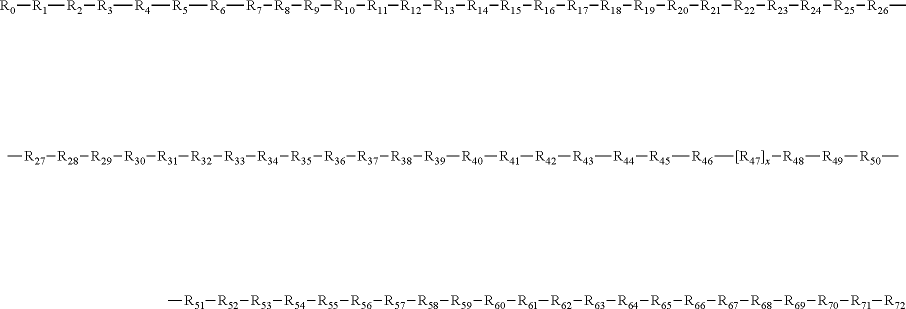

wherein R is a ribonucleotide residue and the consensus for Ser is:
R₀=absent;
R₁₄, R₂₄, R₅₇=are independently A or absent;
R₄₁=A, C or absent;
R₂, R₃, R₄, R₅, R₆, R₇, R₉, R₁₀, R₁, R₁₂, R₁₃, R₁₆, R₂₁, R₂, R₂₆, R₂₇, R₂₈, R₃₀, R₃₁, R₃₂, R₃₃, R₃₄, R₃₇, R₄₂, R₄₃, R₄₄, R₄₅, R₄₆, R₄₈, R₄₉, R₅₀, R₆₂, R₆₃, R₆₄, R₆₅, R₆₆, R₆₇, R₆₈, R₆₉, R₇₀=are independently N or absent;
R₁₈=A, C, U or absent;
R₁₅, R₄₀, R₅₁, R₅₆=are independently A, G or absent;
R₁, R₂₉, R₅₈, R₇₂=are independently A, G, U or absent;
R₃₉=A, U or absent;
R₆₀=C or absent;
R₃₈=C, G or absent;
R₁₇, R₂₂, R₂₃, R₇₁=are independently C, G, U or absent;
R₈, R₃₅, R₃₆, R₅₅, R₅₉, R₆₁=are independently C, U or absent;
R₁₉, R₂₀=are independently G or absent;
R₅₂=G, U or absent;
R₅₃, R₅₄=are independently U or absent;
[R₄₇]ₓ═N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II _{SER}(SEQ ID NO: 608),

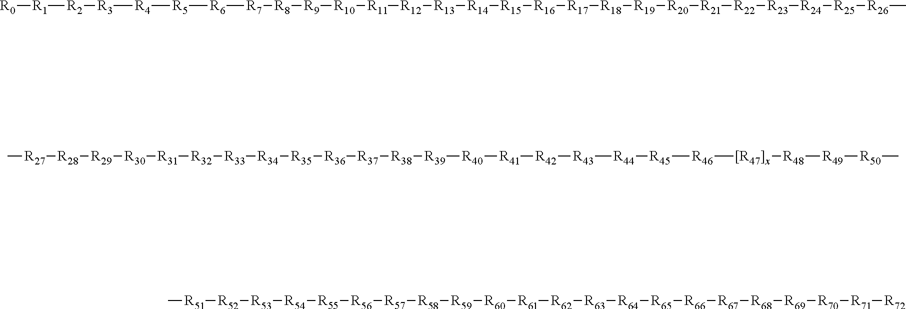

wherein R is a ribonucleotide residue and the consensus for Ser is:
R₀, R₂₃=absent
R₁4, R₂₄, R₄₁, R₅₇=are independently A or absent;
R₄₄=A, C or absent;
R₂₅, R₄₅, R₄₈=are independently A, C, G or absent;
R₂, R₃, R₄, R₅, R₃₇, R₅₀, R₆₂, R₆₆, R₆₇, R₆₉, R₇₀=are independently N or absent;
R₁₂, R₂₃, R₆₅=are independently A, C, U or absent;
R₉, R₁₅, R₂₉, R₃₄, R₄₀, R₅₆, R₆₃=are independently A, G or absent;
R₇, R₂₆, R₃₀, R₃₃, R₄₆, R₅₈, R₇₂=are independently A, G, U or absent;
R₃₉=A, U or absent;
R₁1, R₃₅, R₆₀, R₆₁=are independently C or absent;
R₁₃, R₃₈=are independently C, G or absent;
R₆, R₁₇, R₃₁, R₄₃, R₆₄, R₆₈=are independently C, G, U or absent;
R₃₆, R₄₂, R₄₉, R₅₅, R₅₉, R₇₁=are independently C, U or absent;
R₁₀, R₁₉, R₂₀, R₂₇, R₅₁=are independently G or absent;
R₁, R₁₆, R₃₂, R₅₂=are independently G, U or absent;
R₈, R₁₈, R₂₁, R₂₂, R₅₃, R₅₄=are independently U or absent;
[R₄₇]ₓ═N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III _{SER} (SEQ ID NO: 609),

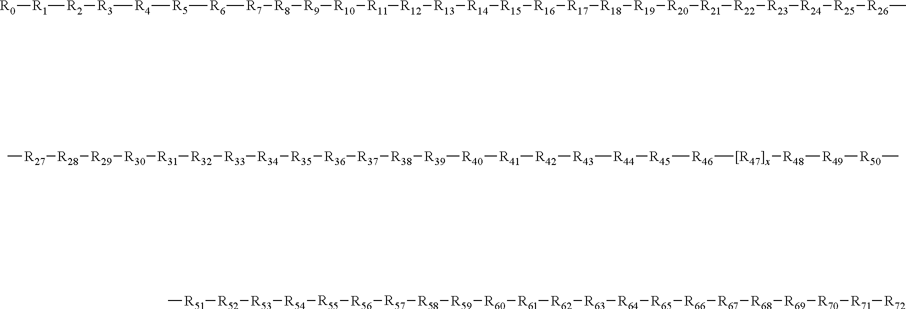

wherein R is a ribonucleotide residue and the consensus for Ser is:
R₀, R₃=absent
R₁₄, R₂₄, R₄₁, R₅₇, R₅₈=are independently A or absent;
R₄₄=A, C or absent;
R₂₅, R₄₈=are independently A, C, G or absent;
R₂, R₃, R₅, R₃₇, R₆₆, R₆₇, R₆₉, R₇₀=are independently N or absent;
R₁₂, R₂₅, R₆₂=are independently A, C, U or absent;
R₇, R₉, R₁₅, R₂₉, R₃, R₃₄, R₄₀, R₄₅, R₅₆, R₆3=are independently A, G or absent;
R₄, R₂₆, R₄₆, R₅₀=are independently A, G, U or absent;
R₃₀, R₃₉=are independently A, U or absent;
R₁1, R₁₇, R₃₅, R₆₀, R₆₁=are independently C or absent;
R₁₃, R₃₈=are independently C, G or absent;
R₆, R₆₄=are independently C, G, U or absent;
R₃₁, R₄₂, R₄₃, R₄₉, R₅₅, R₅₉, R₆₅, R₆₅, R₁=are independently C, U or absent;
R₁₀, R₁9, R₂₀, R₂₇, R₅₁, R₅₂=are independently G or absent;
R₁, R₁₆, R₃₂, R₇₂=are independently G, U or absent;
R₈, R₁₈, R₂₁, R₂₂, R₃₆, R₅₃, R₅₄=are independently U or absent;
[R₄₇]ₓ═N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Threonine TREM Consensus sequence

In an embodiment, a TREM disclosed herein comprises the sequence of Formula I THR (SEQ ID NO: 610),

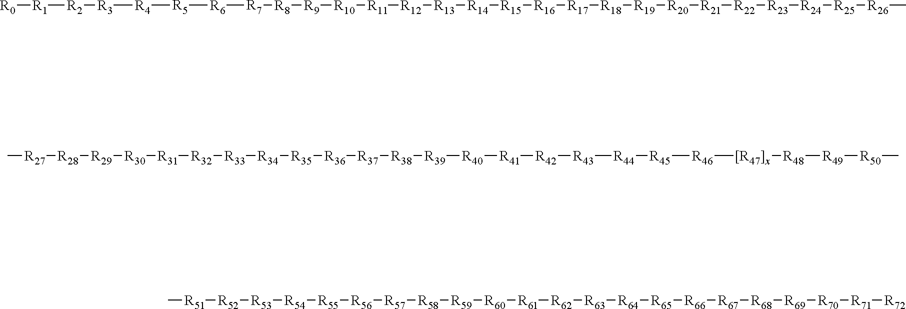

wherein R is a ribonucleotide residue and the consensus for Thr is:
R₀, R₂₃=absent
R₁₄, R₄₁, R₅₇=are independently A or absent;
R₅₆, R₇₀=are independently A, C, G or absent;
R₄, R₅, R₆, R₇, R₁₂, R₁₆, R₂₆, R₃₀, R₃₁, R₃₂, R₃₄, R₃₇, R₄₂, R₄₄, R₄₅, R₄₆, R₄₅, R₄₉, R₅₀, R₅₅, R₆₂, R₆₅, R₆₄, R₆₅, R₆₆, R₆₇, R₆₅, R₇₂=are independently N or absent;
R₁₃, R₁₇, R₂₁, R₃₅, R₆₁=are independently A, C, U or absent;
R₁, R₉, R₂₄, R₂₇, R₂₉, R₆₉=are independently A, G or absent;
R₁₅, R₂₅, R₅₁=are independently A, G, U or absent;
R₄₀, R₅₃=are independently A, U or absent;
R₃₃, R₄₃=are independently C, G or absent;
R₂, R₃, R₅₉=are independently C, G, U or absent;
R₁₁, R₁₈, R₂₂, R₂₅, R₃₆, R₅₄, R₅₅, R₆₀, R₁₁=are independently C, U or absent;
R₁₀, R₂₀, R₃₅, R₅₂=are independently G or absent;
R₁₉=G, U or absent;
R₈, R₃₉=are independently U or absent;
[R₄₇]ₓ═N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II _{T}HR (SEQ ID NO: 611),

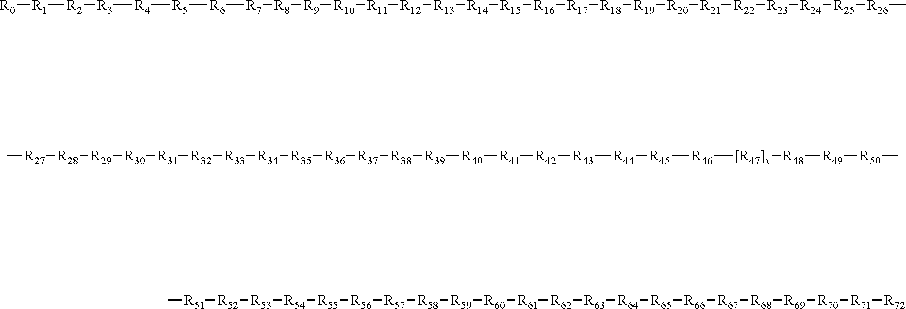

wherein R is a ribonucleotide residue and the consensus for Thr is:
R₀, R₁₅, R₂₃=absent
R₁₄, R₄₁, R₅₇=are independently A or absent;
R₉, R₄₂, R₄₄, R₄₈, R₅₆, R₇₀=are independently A, C, G or absent;
R₄, R₆, R₁₂, R₂₆, R₄₉, R₅₈, R₆₃, R₆₄, R₆₆, R₆₈=are independently N or absent;
R₁₃, R₂₁, R₃1, R₃₇, R₆₂=are independently A, C, U or absent;
R₁, R₁₅, R₂₄, R₂₇, R₂₉, R₄₆, R₅₁, R₆₀=are independently A, G or absent;
R₇, R₂₅, R₄₅, R₅₀, R₆₇=are independently A, G, U or absent;
R₄₀, R₅₃=are independently A, U or absent;
R₃₅=C or absent;
R₃₃, R₄₃=are independently C, G or absent;
R₂, R₃, R₅, R₁₆, R₃₂, R₃₄, R₅₉, R₆₅, R₂₂=are independently C, G, U or absent;
R₁1, R₁7, R₂₂, R₂₈, R₃₀, R₃₆, R₅₅, R₆₀, R₆, R₇₁=are independently C, U or absent;
R₁₀, R₁₉, R₂₀, R₃₈, R₅₂=are independently G or absent;
R₈, R₃₉, R₅₄=are independently U or absent;
[R₄₇]ₓ═N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III _{TIIR} (SEQ ID NO: 612),

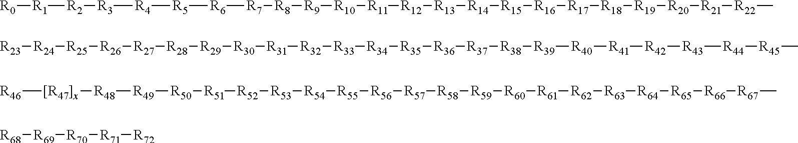

wherein R is a ribonucleotide residue and the consensus for Thr is:
R₀, R₁₈, R₂₃=absent
R₁₄, R₄₀, R₄₁, R₅₇=are independently A or absent;
R₄₄=A, C or absent;
R₉, R₄₂, R₄₅, R₅₆=are independently A, C, G or absent;
R₄, R₆, R₁₂, R₂₆, R₅₈, R₆₄, R₆₆, R₆₈=are independently N or absent;
R₁₃, R₂₁, R₃1, R₃₇, R₄₉, R₆₂=are independently A, C, U or absent;
R₁, R₁₅, R₂₄, R₂₇, R₂₉, R₄₆, R₅₁, R₆₉=are independently A, G or absent;
R₇, R₂₅, R₄₅, R₅₀, R₆₃, R₆₇=are independently A, G, U or absent;
R₅₃=A, U or absent;
R₃₅=C or absent;
R₂, R₃₃, R₄₃, R₇₀=are independently C, G or absent;
R₅, R₁₆, R₃₄, R₅₉, R₆₅=are independently C, G, U or absent;
R₃, R₁₁, R₂₂, R₂₅, R₃₀, R₃₆, R₅₅, R₆₀, R₆₁, R₇₁=are independently C, U or absent;
R₁₀, R₁9, R₂₀, R₃₅, R₅₂=are independently G or absent;
R₃₂=G, U or absent;
R₈, R₁₇, R₃₉, R₅₄, R₇₂=are independently U or absent;
[R₄₇]ₓ═N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Tryptophan TREM Consensus Sequence

In an embodiment, a TREM disclosed herein comprises the sequence of Formula I T_{RP} (SEQ ID NO: 613),

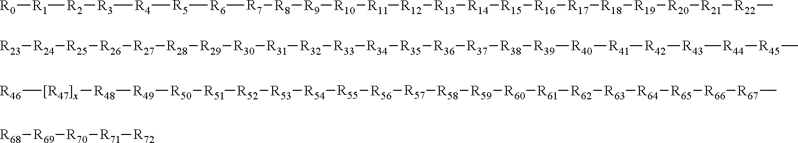

wherein R is a ribonucleotide residue and the consensus for Trp is:
R₀=absent;
R₂₄, R₃₉, R₄₁, R₅₇=are independently A or absent;
R₂, R₃, R₂₆, R₂₇, R₄₀, R₄₈=are independently A, C, G or absent;
R₄, R₅, R₆, R₂₉, R₃₀, R₃₁, R₃₂, R₃₄, R₄₂, R₄₄, R₄₅, R₄₆, R₄₉, R₅₁, R₅₈, R₆₃, R₆₆, R₆₇, R₆₈=are independently N or absent;
R₁₃, R₁₄, R₁₆, R₁₈, R₂₁, R₆₁, R₆₅, R₇₁=are independently A, C, U or absent;
R₁, R₉, R₁₀, R₁₅, R₃₃, R₅₀, R₅₆=are independently A, G or absent;
R₇, R₂₅, R₂=are independently A, G, U or absent;
R₃₇, R₃₅, R₅₅, R₆₀=are independently C or absent;
R₁2, R₃₅, R₄₃, R₆₄, R₆₉, R₇₀=are independently C, G, U or absent;
R₁₁, R₁₇, R₂₂, R₂₈, R₅₉, R₆₂=are independently C, U or absent;
R₁₉, R₂₀, R₅₂=are independently G or absent;
R₈, R₂₃, R₃₆, R₅₃, R₅₄=are independently U or absent;
[R₄₇]ₓ═N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II T_{RP}(SEQ ID NO: 614),

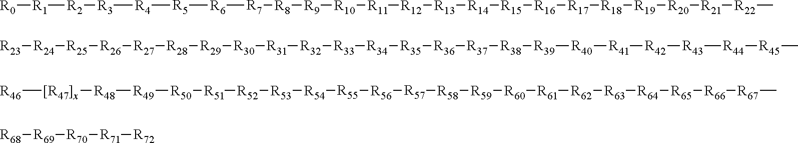

wherein R is a ribonucleotide residue and the consensus for Trp is:
R₀, R₁₅, R₂₂, R₂₃=absent
R₁₄, R₂₄, R₃₉, R₄₁, R₅₇, R₇₂=are independently A or absent;
R₃, R₄, R₁₃, R₆₁, R₇₁=are independently A, C or absent;
R₆, R₄₄=are independently A, C, G or absent;
R₂₁=A, C, U or absent;
R₂, R₇, R₁₅, R₂₅, R₃₃, R₃₄, R₄₅, R₅₆, R₆₃=are independently A, G or absent;
R₅₈=A, G, U or absent;
R₄₆=A, U or absent;
R₃₇, R₃₅, R₅₅, R₆₀, R₆₂=are independently C or absent;
R₁₂, R₂₆, R₂₇, R₃₅, R₄₀, R₄₈, R₆₇=are independently C, G or absent;
R₃₂, R₄₃, R₆₈=are independently C, G, U or absent;
R₁₁, R₁₆, R₂₈, R₃₁, R₄₉, R₅₉, R₆₅, R₇₀=are independently C, U or absent;
R₁, R₉, R₁₀, R₁₉, R₂₀, R₅₀, R₅₂, R₆₉=are independently G or absent;
R₅, R₈, R₂₉, R₃₀, R₄₂, R₅₁, R₆₄, R₆₆=are independently G, U or absent;
R₁₇, R₃₆, R₅₃, R₅₄=are independently U or absent;
[R₄₇]ₓ═N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III_{TRP} (SEQ ID NO: 615),

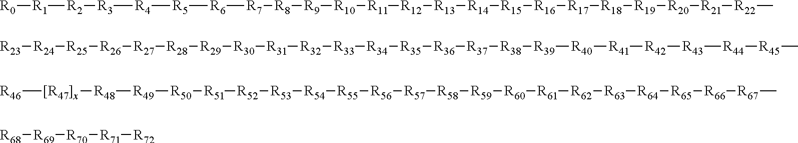

wherein R is a ribonucleotide residue and the consensus for Trp is:
R₀, R₁, R₂₂, R₂₃=absent
R₁₄, R₂₄, R₃₉, R₄₁, R₅₇, R₇₂=are independently A or absent;
R₃, R₄, R₁₃, R₆₁, R₇₁=are independently A, C or absent;
R₆, R₄₄=are independently A, C, G or absent;
R₂=A, C, U or absent;
R₂, R₇, R₁₅, R₂₅, R₃₃, R₃₄, R₄₅, R₅₆, R₆₃=are independently A, G or absent;
R₅₈=A, G, U or absent;
R₄₆=A, U or absent;
R₃₇, R₃₈, R₅₅, R₆₀, R₆₂=are independently C or absent;
R₁₂, R₂₆, R₂₇, R₃₅, R₄₀, R₄₈, R₆₇=are independently C, G or absent;
R₃₂, R₄₃, R₆₈=are independently C, G, U or absent;
R₁₁, R₁₆, R₂₈, R₃₁, R₄₉, R₅₉, R₆₅, R₇₀=are independently C, U or absent;
R₁, R₉, R₁₀, R₁₉, R₂₀, R₅₀, R₅₂, R₆₉=are independently G or absent;
R₅, R₈, R₂₉, R₃₀, R₄₂, R₅₁, R₆₄, R₆₆=are independently G, U or absent;
R₁₇, R₃₆, R₅₃, R₅₄=are independently U or absent;
[R₄₇]ₓ═N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Tyrosine TREM Consensus Sequence

In an embodiment, a TREM disclosed herein comprises the sequence of Formula I _{TYR} (SEQ ID NO: 616),

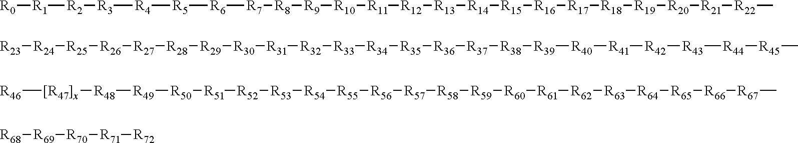

wherein R is a ribonucleotide residue and the consensus for Tyr is:
R₀=absent
R₁₄, R₃₉, R₅₇=are independently A or absent;
R₄₁, R₄₈, R₅₁, R₇₁=are independently A, C, G or absent;
R₃, R₄, R₅, R₆, R₉, R₁₀, R₁₂, R₁₃, R₁₆, R₂₅, R₂₆, R₃₀, R₃₁, R₃₂, R₄₂, R₄₄, R₄₅, R₄₆, R₄₉, R₅₀, R₅₅, R₂, R₃, R₆₆, R₆₇, R₆₅, R₆₉, R₇₀=are independently N or absent;
R₂₂, R₆₅=are independently A, C, U or absent;
R₁₅, R₂₄, R₂₇, R₃₃, R₃₇, R₄₀, R₅₆=are independently A, G or absent;
R₇, R₂₉, R₃₄, R₇₂=are independently A, G, U or absent;
R₂₃, R₅₃=are independently A, U or absent;
R₃₅, R₆₀=are independently C or absent;
R₂₀=C, G or absent;
R₁, R₂, R₂₈, R₆₁, R₆₄=are independently C, G, U or absent;
R₁1, R₁₇, R₂₁, R₄₃, R₅₅=are independently C, U or absent;
R₁₉, R₅₂=are independently G or absent;
R₈, R₁₈, R₃₆, R₃₈, R₅₄, R₅₉=are independently U or absent;
[R₄₇]ₓ═N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II _{TYR}(SEQ ID NO: 617),

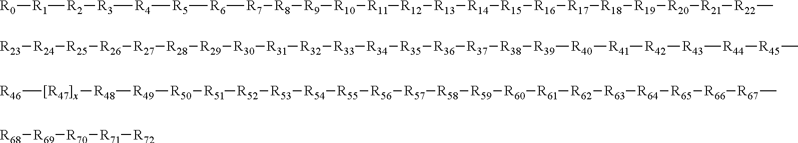

wherein R is a ribonucleotide residue and the consensus for Tyr is:
R₀, R₁₈, R₂₃=absent
R₇, R₉, R₁₄, R₂₄, R₂₆, R₃₄, R₃₉, R₅₇=are independently A or absent;
R₄₄, R₆₉=are independently A, C or absent;
R₇₁=A, C, G or absent;
R₆₈=N or absent;
R₅₈=A, C, U or absent;
R₃₃, R₃₇, R₄₁, R₅₆, R₆₂, R₆₃=are independently A, G or absent;
R₆, R₂₉, R₇₂=are independently A, G, U or absent;
R₃₁, R₄₅, R₅₃=are independently A, U or absent;
R₁₃, R₃₅, R₄₉, R₆₀=are independently C or absent;
R₂₀, R₄₈, R₆₄, R₆₇, R₇₀=are independently C, G or absent;
R₁, R₂, R₅, R₁₆, R₆₆=are independently C, G, U or absent;
R₁₁, R₂₁, R₂₅, R₄₃, R₅₅, R₆₁=are independently C, U or absent;
R₁₀,R₁5,R₉,R₂₅,R₂₇,R₄₀,R₅₁,R₅₂=are independently G or absent;
R₃,R₄,R₃₀,R₃₂,R₄₂,R₄₆=are independently G, U or absent;
R₈,R₁₂,R₁₇,R₂₂,R₃₆,R₃₈,R₅₀,R₅₄,R₅₉,R₆₅=are independently U or absent;
[R₄₇]ₓ═N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III TYR (SEQ ID NO: 618),

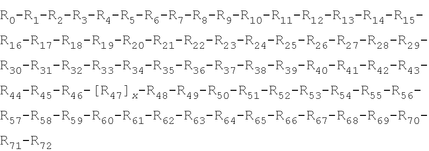

wherein R is a ribonucleotide residue and the consensus for Tyr is:
R₀,R₁,R₂₃=absent
R₇,R₉,R₁₄,R₂₄,R₂₆,R₃₄,R₃₉,R₅₇,R₂=are independently A or absent;
R₄₄,R₆₉=are independently A, C or absent;
R₇₁=A, C, G or absent;
R₃₇,R₄₁,R₅₆,R₆₂,R₆₃=are independently A, G or absent;
R₆,R₂₉,R₆₈=are independently A, G, U or absent;
R₃₁,R₄₅,R₅₈=are independently A, U or absent;
R₁₃,R₂₈,R₃₅,R₄₉,R₆₀,R₆₁=are independently C or absent;
R₅,R₄₈,R₆₄,R₆₇,R₇₀=are independently C, G or absent;
R₁,R₂=are independently C, G, U or absent;
R₁1,R₁₆,R₂₁,R₄₃,R₅₅,R₆₆=are independently C, U or absent;
R₁₀,R₁₅,R₁₉,R₂₀,R₂₅,R₂₇,R₃₃,R₄₀,R₅₁,R₅₂=are independently G or absent;
R₃,R₄,R₃₀,R₃₂,R₄₂,R₄₆=are independently G, U or absent;
R₈,R₁₂,R₁₇,R₂₂,R₃₆,R₃₈,R₅₀,R₅₃,R₅₄,R₅₉,R₆₅=are independently U or absent;
[R₄₇]ₓ═N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Valine TREM Consensus Sequence

In an embodiment, a TREM disclosed herein comprises the sequence of Formula I VAL (SEQ ID NO: 619),

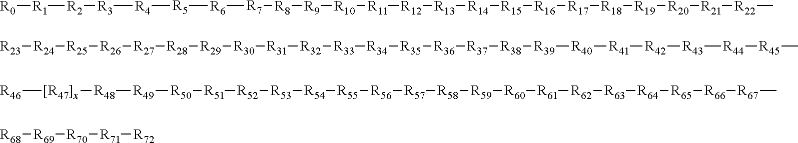

wherein R is a ribonucleotide residue and the consensus for Val is:
R₀,R₂₃=absent;
R₂₄,R₃₅,R₅₇=are independently A or absent;
R₉,R₇₂=are independently A, C, G or absent;
R₂,R₄,R₅,R₆,R₇,R₁₂,R₁₅,R₁₆,R₂₁,R₂₅,R₂₆,R₂₉,R₃₁,R₃₂,R₃₃,R₃₄,R₃₇,R₄₁,R₄₂,R₄₃,R₄₄,R₄₅,R₄₆,R₄₈,R₄₉,R₅₀,R₅₈,R₆₁,R₆₂,R₆₃,R₆₄,R₆₅,R₆₆,R₆₇,R₆₈,R₆₉,R₇₀=are independently N or absent;
R₁₇,R₃₅,R₅₉=are independently A, C, U or absent;
R₁₀,R₁₄,R₂₇,R₄₀,R₅₂,R₅₆=are independently A, G or absent;
R₁,R₃,R₅₁,R₅₃=are independently A, G, U or absent;
R₃₉=C or absent;
R₁₃,R₃₀,R₅₅=are independently C, G, U or absent;
R₁1,R₂₂,R₂₈,R₆₀,R₇₁=are independently C, U or absent;
R₁₉=G or absent;
R₂₀=G, U or absent;
R₈,R₁₈,R₃₆,R₅₄=are independently U or absent;
[R₄₇]ₓ═N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II _{VAL} (SEQ ID NO: 620),

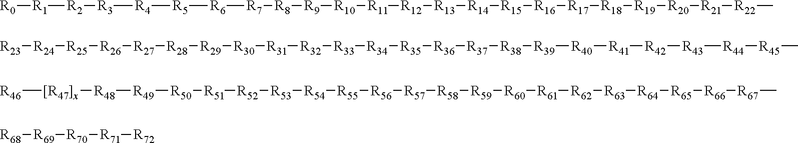

wherein R is a ribonucleotide residue and the consensus for Val is:
R₀,R₁₅,R₂₃=absent;
R₂₄,R₃₅,R₅₇=are independently A or absent;
R₆₄,R₇₀,R₇₂=are independently A, C, G or absent;
R₁₅,R₁₆,R₂₆,R₂₉,R₃₁,R₃₂,R₄₃,R₄₄,R₄₅,R₄₉,R₅₀,R₅₈,R₆₂,R₆₅=are independently N or absent;
R₆,R₁₇,R₃₄,R₃₇,R₄₁,R₅₉=are independently A, C, U or absent;
R₉,R₁₀,R₁₄,R₂₇,R₄₀,R₄₆,R₅₁,R₅₂,R₅₆=are independently A, G or absent;
R₇,R₁₂,R₂₅,R₃₃,R₅₃,R₆₃,R₆₆,R₆₈=are independently A, G, U or absent;
R₆₉=A, U or absent;
R₃₉=C or absent;
R₅,R₆₇=are independently C, G or absent;
R₂,R₄,R₁₃,R₄₈,R₅₅,R₆₁=are independently C, G, U or absent;
R₁1,R₂₂,R₂₈,R₃₀,R₃₅,R₆₀,R₇₁=are independently C, U or absent;
R₁₉=G or absent;
R₁,R₃,R₂₀,R₄₂=are independently G, U or absent;
R₈,R₂₁,R₃₆,R₅₄=are independently U or absent;
[R₄₇]ₓ═N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III _{VAL}(SEQ ID NO: 621),

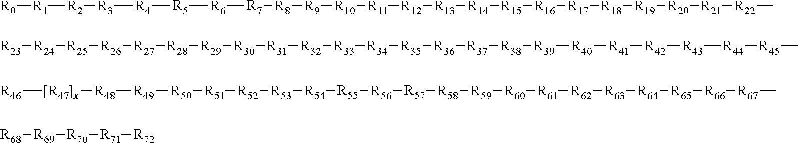

wherein R is a ribonucleotide residue and the consensus for Val is:
R₀,R₁₅,R₂₃=absent
R₂₄,R₃₈,R₄₀,R₅₇,R₇₂=are independently A or absent;
R₂₉,R₆₄,R₇₀=are independently A, C, G or absent;
R₄₉,R₅₀,R₆₂=are independently N or absent;
R₁₆,R₂₆,R₃1,R₃₂,R₃₇,R₄₁,R₄₃,R₅₉,R₆₅=are independently A, C, U or absent;
R₉,R₁₄,R₂₇,R₄₆,R₅₂,R₅₆,R₆₆=are independently A, G or absent;
R₇,R₁₂,R₂₅,R₃₃,R₄₄,R₄₅,R₅₃,R₅₈,R₆₃,R₆₈=are independently A, G, U or absent;
R₆₉=A, U or absent;
R₃₉=C or absent;
R₅,R₆₇=are independently C, G or absent;
R₂,R₄,R₁₃,R₁₅,R₄₈,R₅₅=are independently C, G, U or absent;
R₆,R₁1,R₂₂,R₂₈,R₃₀,R₃₄,R₃₅,R₆₀,R₆₁,R₇₁=are independently C, U or absent;
R₁₀,R₁₉,R₅₁=are independently G or absent;
R₁,R₃,R₂₀,R₄₂=are independently G, U or absent;
R₈,R₁₇,R₂₁,R₃₆,R₅₄=are independently U or absent;
[R₄₇]ₓ═N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Variable Region Consensus Sequence

In an embodiment, a TREM disclosed herein comprises a variable region at position R₄₇.

In an embodiment, the variable region is 1-271 ribonucleotides in length (e.g. 1-250, 1-225, 1-200, 1-175, 1-150, 1-125, 1-100, 1-75, 1-50, 1-40, 1-30, 1-29, 1-28, 1-27, 1-26, 1-25, 1-24, 1-23, 1-22, 1-21, 1-20, 1-19, 1-18, 1-17, 1-16, 1-15, 1-14, 1-13, 1-12, 1-11, 1-10, 10-271, 20-271, 30-271, 40-271, 50-271, 60-271, 70-271, 80-271, 100-271, 125-271, 150-271, 175-271, 200-271, 225-271, 1,2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 110, 125, 150, 175, 200, 225, 250, or 271 ribonucleotides). In an embodiment, the variable region comprises any one, all or a combination of Adenine, Cytosine, Guanine or Uracil.

In an embodiment, the variable region comprises a ribonucleic acid (RNA) sequence encoded by a deoxyribonucleic acid (DNA) sequence disclosed in Table 4, e.g., any one of SEQ ID NOs: 452-561 disclosed in Table 4.

TABLE 4
Exemplary variable region sequences.

| | SEQ ID NO | SEQUENCE |
|---|---|---|
| 1 | 452 | AAAATATAAATATATTTC |
| 2 | 453 | AAGCT |
| 3 | 454 | AAGTT |
| 4 | 455 | AATTCTTCGGAATGT |
| 5 | 456 | AGA |
| 6 | 457 | AGTCC |
| 7 | 458 | CAACC |
| 8 | 459 | CAATC |
| 9 | 460 | CAGC |
| 10 | 461 | CAGGCGGGTTCTGCCCGCGC |
| 11 | 462 | CATACCTGCAAGGGTATC |
| 12 | 463 | CGACCGCAAGGTTGT |
| 13 | 464 | CGACCTTGCGGTCAT |
| 14 | 465 | CGATGCTAATCACATCGT |
| 15 | 466 | CGATGGTGACATCAT |
| 16 | 467 | CGATGGTTTACATCGT |
| 17 | 468 | CGCCGTAAGGTGT |
| 18 | 469 | CGCCTTAGGTGT |
| 19 | 470 | CGCCTTTCGACGCGT |
| 20 | 471 | CGCTTCACGGCGT |
| 21 | 472 | CGGCAGCAATGCTGT |
| 22 | 473 | CGGCTCCGCCTTC |
| 23 | 474 | CGGGTATCACAGGGTC |
| 24 | 475 | CGGTGCGCAAGCGCTGT |
| 25 | 476 | CGTACGGGTGACCGTACC |
| 26 | 477 | CGTCAAAGACTTC |
| 27 | 478 | CGTCGTAAGACTT |
| 28 | 479 | CGTTGAATAAACGT |
| 29 | 480 | CTGTC |
| 30 | 481 | GGCC |
| 31 | 482 | GGGGATT |
| 32 | 483 | GGTC |
| 33 | 484 | GGTTT |
| 34 | 485 | GTAG |
| 35 | 486 | TAACTAGATACTTTCAGAT |
| 36 | 487 | TACTCGTATGGGTGC |
| 37 | 488 | TACTTTGCGGTGT |
| 38 | 489 | TAGGCGAGTAACATCGTGC |
| 39 | 490 | TAGGCGTGAATAGCGCCTC |
| 40 | 491 | TAGGTCGCGAGAGCGGCGC |
| 41 | 492 | TAGGTCGCGTAAGCGGCGC |
| 42 | 493 | TAGGTGGTTATCCACGC |
| 43 | 494 | TAGTC |
| 44 | 495 | TAGTT |
| 45 | 496 | TATACGTGAAAGCGTATC |
| 46 | 497 | TATAGGGTCAAAAACTCTATC |
| 47 | 498 | TATGCAGAAATACCTGCATC |
| 48 | 499 | TCCCCATACGGGGGC |
| 49 | 500 | TCCCGAAGGGGTTC |
| 50 | 501 | TCTACGTATGTGGGC |
| 51 | 502 | TCTCATAGGAGTTC |
| 52 | 503 | TCTCCTCTGGAGGC |
| 53 | 504 | TCTTAGCAATAAGGT |
| 54 | 505 | TCTTGTAGGAGTTC |
| 55 | 506 | TGAACGTAAGTTCGC |
| 56 | 507 | TGAACTGCGAGGTTCC |
| 57 | 508 | TGAC |
| 58 | 509 | TGACCGAAAGGTCGT |
| 59 | 510 | TGACCGCAAGGTCGT |
| 60 | 511 | TGAGCTCTGCTCTC |
| 61 | 512 | TGAGGCCTCACGGCCTAC |
| 62 | 513 | TGAGGGCAACTTCGT |
| 63 | 514 | TGAGGGTCATACCTCC |
| 64 | 515 | TGAGGGTGCAAATCCTCC |
| 65 | 516 | TGCCGAAAGGCGT |
| 66 | 517 | TGCCGTAAGGCGT |
| 67 | 518 | TGCGGTCTCCGCGC |
| 68 | 519 | TGCTAGAGCAT |
| 69 | 520 | TGCTCGTATAGAGCTC |
| 70 | 521 | TGGACAATTGTCTGC |
| 71 | 522 | TGGACAGATGTCCGT |
| 72 | 523 | TGGACAGGTGTCCGC |
| 73 | 524 | TGGACGGTTGTCCGC |
| 74 | 525 | TGGACTTGTGGTC |
| 75 | 526 | TGGAGATTCTCTCCGC |
| 76 | 527 | TGGCATAGGCCTGC |
| 77 | 528 | TGGCTTATGTCTAC |
| 78 | 529 | TGGGAGTTAATCCCGT |
| 79 | 530 | TGGGATCTTCCCGC |
| 80 | 531 | TGGGCAGAAATGTCTC |
| 81 | 532 | TGGGCGTTCGCCCGC |
| 82 | 533 | TGGGCTTCGCCCGC |
| 83 | 534 | TGGGGGATAACCCCGT |
| 84 | 535 | TGGGGGTTTCCCCGT |
| 85 | 536 | TGGT |
| 86 | 537 | TGGTGGCAACACCGT |
| 87 | 538 | TGGTTTATAGCCGT |
| 88 | 539 | TGTACGGTAATACCGTACC |
| 89 | 540 | TGTCCGCAAGGACGT |
| 90 | 541 | TGTCCTAACGGACGT |
| 91 | 542 | TGTCCTATTAACGGACGT |
| 92 | 543 | TGTCCTTCACGGGCGT |
| 93 | 544 | TGTCTTAGGACGT |
| 94 | 545 | TGTGCGTTAACGCGTACC |
| 95 | 546 | TGTGTCGCAAGGCACC |
| 96 | 547 | TGTTCGTAAGGACTT |
| 97 | 548 | TTCACAGAAATGTGTC |
| 98 | 549 | TTCCCTCGTGGAGT |
| 99 | 550 | TTCCCTCTGGGAGC |
| 100 | 551 | TTCCCTTGTGGATC |
| 101 | 552 | TTCCTTCGGGAGC |
| 102 | 553 | TTCTAGCAATAGAGT |
| 103 | 554 | TTCTCCACTGGGGAGC |
| 104 | 555 | TTCTCGAGAGGGAGC |
| 105 | 556 | TTCTCGTATGAGAGC |
| 106 | 557 | TTTAAGGTTTTCCCTTAAC |
| 107 | 558 | TTTCATTGTGGAGT |
| 108 | 559 | TTTCGAAGGAATCC |
| 109 | 560 | TTTCTTCGGAAGC |
| 110 | 561 | TTTGGGGCAACTCAAC |

Corresponding Nucleotide Positions To determine if a selected nucleotide position in a candidate sequence corresponds to a selected position in a reference sequence (e.g., SEQ ID NO: 622, SEQ ID NO: 623, SEQ ID NO: 624), one or more of the following Evaluations is performed.

### Evaluation A:

1. The candidate sequence is aligned with each of the consensus sequences in Tables 9 and 10. The consensus sequence(s) having the most positions aligned (and which has at least 60% of the positions of the candidate sequence aligned) is selected.

The alignment is performed as is follows. The candidate sequence and an isodecoder consensus sequence from Tables 10A-10B are aligned based on a global pairwise alignment calculated with the Needleman-Wunsch algorithm when run with match scores from Table 11, a mismatch penalty of −1, a gap opening penalty of −1, and a gap extension penalty of −0.5, and no penalty for end gaps. The alignment with the highest overall alignment score is then used to determine the percent similarity between the candidate and the consensus sequence by counting the number of matched positions in the alignment, dividing it by the larger of the number of non-N bases in the candidate sequence or the consensus sequence, and multiplying the result by 100. In cases where multiple alignments (of the candidate and a single consensus sequence) tie for the same score, the percent similarity is the largest percent similarity calculated from the tied alignments. This process is repeated for the candidate sequence with each of the remaining isodecoder consensus sequences in Tables 10A-10B, and the alignment resulting in the greatest percent similarity is selected. If this alignment has a percent similarity equal to or greater than 60%, it is considered a valid alignment and used to relate positions in the candidate sequence to those in the consensus sequence, otherwise the candidate sequence is considered to have not aligned to any of the isodecoder consensus sequences. If there is a tie at this point, all tied consensus sequences are taken forward to step 2 in the analysis.
2. Using the selected consensus sequence(s) from step 1, one determines the consensus sequence position number that aligns with the selected position (e.g., a modified position) in the candidate sequence. One then assigns the position number of the aligned position in the consensus sequence to the selected position in the candidate sequence, in other words, the selected position in the candidate sequence is numbered according to the numbering of the consensus sequence. If there were tied consensus sequences from step one, and they give different position numbers in this step 2, then all such position numbers are taken forward to step 5.
3. The reference sequence is aligned with the consensus sequence chosen in step 1. The alignment is performed as described in step 1.
4. From the alignment in step 3, one determines the consensus sequence position number that aligns with the selected position (e.g., a modified position) in the reference sequence. One then assigns the position number of the aligned position in the consensus sequence to the selected position in the reference sequence, in other words, the selected position in the reference sequence is numbered according to the numbering of the consensus sequence. If there is a tie at this point, all tied consensus sequences are taken forward to step 5 in the analysis.
5. If a value for a position number determined for the reference sequence in step 2 is the same as the value for the position number determined for the candidate sequence in step 4, the positions are defined as corresponding.

### Evaluation B:

The reference sequence (e.g., a TREM sequence described herein) and the candidate sequence are aligned with one another. The alignment is performed as follows.

The reference sequence and the candidate sequence are aligned based on a global pairwise alignment calculated with the Needleman-Wunsch algorithm when run with match scores from Table 11, a mismatch penalty of −1, a gap opening penalty of −1, and a gap extension penalty of −0.5, and no penalty for end gaps. The alignment with the highest overall alignment score is then used to determine the percent similarity between the candidate and reference sequence by counting the number of matched based in the alignment, dividing it by the larger of the number of non-N bases in the candidate or reference sequence, and multiplying the result by 100. In cases where multiple alignments tie for the same score, the percent similarity is the largest percent similarity calculated from the tied alignments. If this alignment has a percent similarity equal to or greater than 60%, it is considered a valid alignment and used to relate positions in the candidate sequence to those in the reference sequence, otherwise the candidate sequence is considered to have not aligned to the reference sequence.

If the selected nucleotide position in the reference sequence (e.g., a modified position) is paired with a selected nucleotide position (e.g., a modified position) in the candidate sequence, the positions are defined as corresponding.

### Evaluation C:

The candidate sequence is assigned a nucleotide position number according to the comprehensive tRNA numbering system (CtNS), also referred to as the tRNAviz method (e.g., as described in Lin et al., Nucleic Acids Research, 47:W1, pages W542-W547, 2 Jul. 2019), which serves as a global numbering system for tRNA molecules. The alignment is performed as follows.
1. The candidate sequence is assigned a nucleotide position according to the tRNAviz method. For a novel sequence not present in the tRNAviz database, the numbering for the closest sequence in the database is obtained. For example, if a TREM differs at any given nucleotide position from a sequence in the database, the numbering for the tRNA having the wildtype sequence at said given nucleotide position is used.
2. The reference sequence is assigned a nucleotide position according to the method described in 1.
3. If a value for a position number determined for the reference sequence in step 1 is the same as the value for the position number determined for the candidate sequence in step 2, the positions are defined as corresponding.

If the selected position in the reference sequence and the candidate sequence are found to be corresponding in at least one of Evaluations A, B, and C, the positions correspond. For example, if two positions are found to be corresponding under Evaluation A, but do not correspond under Evaluation B or Evaluation C, the positions are defined as corresponding. Similarly, if two positions are found to be corresponding under Evaluation B, but do not correspond under Evaluation A or Evaluation C, the positions are defined as corresponding. In addition, if two positions are found to be corresponding under Evaluation C, but do not correspond under Evaluation A or Evaluation B, the positions are defined as corresponding

The numbering given above is used for ease of presentation and does not imply a required sequence. If more than one Evaluation is performed, they can be performed in any order.

TABLE 10A
Consensus sequence computationally generated for each isodecoder by aligning
members of the isodecoder family

| SEQ ID | Amino | | |
|---|---|---|---|
| NO. | Acid | Anticodon | Consensus sequence |
| 1200 | Ala | AGC | GGGGAATTAGCTCAAGTGGTAGAGCGCTTG |
| | | | CTTAGCATGCAAGAGGTAGTGGGATCGATG |
| | | | CCCACATTCTCCA |
| 1201 | Ala | CGC | GGGGATGTAGCTCAGTGGTAGAGCGCATGC |
| | | | TTCGCATGTATGAGGTCCCGGGTTCGATCCC |
| | | | CGGCATCTCCA |
| 1202 | Ala | TGC | GGGGGTGTAGCTCAGTGGTAGAGCGCATGC |
| | | | TTTGCATGTATGAGGCCCCGGGTTCGATCCC |
| | | | CGGCACCTCCA |
| 1203 | Arg | ACG | GGGCCAGTGGCGCAATGGATAACGCGTCTG |
| | | | ACTACGGATCAGAAGATTCCAGGTTCGACTC |
| | | | CTGGCTGGCTCG |
| 1204 | Arg | CCG | GGCCGCGTGGCCTAATGGATAAGGCGTCTG |
| | | | ATTCCGGATCAGAAGATTGAGGGTTCGAGTC |
| | | | CCTTCGTGGTCG |
| 1205 | Arg | CCT | GCCCCAGTGGCCTAATGGATAAGGCACTGG |
| | | | CCTCCTAAGCCAGGGATTGTGGGTTCGAGTC |
| | | | CCACCTGGGGTA |
| 1206 | Arg | TCG | GACCGCGTGGCCTAATGGATAAGGCGTCTG |
| | | | ACTTCGGATCAGAAGATTGAGGGTTCGAGTC |
| | | | CCTCCGTGGTCG |
| 1207 | Arg | TCT | GGCTCTGTGGCGCAATGGATNAGCGCATTG |
| | | | GACTTCTAATTCAAAGGTTGCGGGTTCGAGT |
| | | | CCCNCCAGAGTCG |
| 1208 | Asn | GTT | GTCTCTGTGGCGCAATCGGTTAGCGCGTTCG |
| | | | GCTGTTAACCGNAAAGGTTGGTGGTTCGAGC |
| | | | CCACCCAGGGACG |
| 1209 | Asp | GTC | TCCTCGTTAGTATAGTGGTGAGTATCCCCGC |
| | | | CTGTCACGCGGGAGACCGGGGTTCGATTCCC |
| | | | CGACGGGGAG |
| 1210 | Cys | GCA | GGGGGTATAGCTCAGNGGGTAGAGCATTTG |
| | | | ACTGCAGATCAAGAGGTCCCCGGTTCAAATC |
| | | | CGGGTGCCCCCT |
| 1211 | Gln | CTG | GGTTCCATGGTGTAATGGTNAGCACTCTGGA |
| | | | CTCTGAATCCAGCGATCCGAGTTCAAGTCTC |
| | | | GGTGGAACCT |
| 1212 | Gln | TTG | GGTCCCATGGTGTAATGGTTAGCACTCTGGA |
| | | | CTTTGAATCCAGCGATCCGAGTTCAAATCTC |
| | | | GGTGGGACCT |
| 1213 | Glu | CTC | TCCCTGGTGGTCTAGTGGTTAGGATTCGGCG |
| | | | CTCTCACCGCCGCGGCCCGGGTTCGATTCCC |
| | | | GGTCAGGGAA |
| 1214 | Glu | TTC | TCCCTGGTGGTCTAGTGGCTAGGATTCGGCG |
| | | | CTTTCACCGCNGCGGCCCGGGTTCGATTCCC |
| | | | GGTCAGGGAA |
| 1215 | Gly | CCC | GCATTGGTGGTTCAGTGGTAGAATTCTCGCC |
| | | | TCCCACGCNGGAGACCCGGGTTCGATTCCCG |
| | | | GCCAATGCA |
| 1216 | Gly | GCC | GCATTGGTGGTTCAGTGGTAGAATTCTCGCC |
| | | | TGCCACGCGGGAGGCCCGGGTTCGATTCCCG |
| | | | GCCAATGCA |
| 1217 | Gly | TCC | GCGTTGGTGGTATAGTGGTGAGCATAGCTGC |
| | | | CTTCCAAGCAGTTGACCCGGGTTCGATTCCC |
| | | | GGCCAACGCA |
| 1218 | Ile | AAT | GGCCGGTTAGCTCAGTTGGTTAGAGCGTGGT |
| | | | GCTAATAACGCCAAGGTCGCGGGTTCGATCC |
| | | | CCGTACGGGCCA |
| 1219 | Ile | TAT | GCTCCAGTGGCGCAATCGGTTAGCGCGCGGT |
| | | | ACTTATAATGCCGAGGTTGTGAGTTCGAGCC |
| | | | TCACCTGGAGCA |
| 1220 | Leu | AAG | GGTAGCGTGGCCGAGCGGTCTAAGGCGCTG |
| | | | GATTAAGGCTCCAGTCTCTTCGGGGGCGTGG |
| | | | GTTCGAATCCCACCGCTGCCA |
| 1221 | Leu | CAA | GTCAGGATGGCCGAGTGGTCNTAAGGCGCC |
| | | | AGACTCAAGTTCTGGTCTCCGNATGGAGGCG |
| | | | TGGGTTCGAATCCCACTTCTGACA |
| 1222 | Leu | CAG | GTCAGGATGGCCGAGCGGTCTAAGGCGCTG |
| | | | CGTTCAGGTCGCAGTCTCCCCTGGAGGCGTG |
| | | | GGTTCGAATCCCACTCCTGACA |
| 1223 | Leu | TAA | ACCAGGATGGCCGAGTGGTTAAGGCGTTGG |
| | | | ACTTAAGATCCAATGGACAGATGTCCGCGTG |
| | | | GGTTCGAACCCCACTCCTGGTA |
| 1224 | Leu | TAG | GGTAGCGTGGCCGAGCGGTCTAAGGCGCTG |
| | | | GATTTAGGCTCCAGTCTCTTCGGNGGCGTGG |
| | | | GTTCGAATCCCACCGCTGCCA |
| 1225 | Lys | CTT | GCCCGGCTAGCTCAGTCGGTAGAGCATGAG |
| | | | ACTCTTAATCTCAGGGTCGTGGGTTCGAGCC |
| | | | CCACGTTGGGCGNNN |
| 1226 | Lys | TTT | GCCTGGATAGCTCAGTCGGTAGAGCATCAG |
| | | | ACTTTTAATCTGAGGGTCCAGGGTTCAAGTC |
| | | | CCTGTTCAGGCG |
| 1227 | Met | CAT | GCCCTCTTAGCGCAGTNGGCAGCGCGTCAGT |
| | | | CTCATAATCTGAAGGTCCTGAGTTCGAGCCT |
| | | | CAGAGAGGGCA |
| 1228 | Phe | GAA | GCCGAAATAGCTCAGTTGGGAGAGCGTTAG |
| | | | ACTGAAGATCNTAAAGGTCCCTGGTTCAATC |
| | | | CCGGGTTTCGGCA |
| 1229 | Pro | AGG | GGCTCGTTGGTCTAGGGGTATGATTCTCGCT |
| | | | TAGGATGCGAGAGGTCCCGGGTTCAAATCC |
| | | | CGGACGAGCCC |
| 1230 | Pro | CGG | GGCTCGTTGGTCTAGGGGTATGATTCTCGCT |
| | | | TCGGGTGCGAGAGGTCCCGGGTTCAAATCCC |
| | | | GGACGAGCCC |
| 1231 | Pro | TGG | GGCTCGTTGGTCTAGGGGTATGATTCTCGCT |
| | | | TTGGGTGCGAGAGGTCCCGGGTTCAAATCCC |
| | | | GGACGAGCCC |
| 1232 | Ser | AGA | GTAGTCGTGGCCGAGTGGTTAAGGCGATGG |
| | | | ACTAGAAATCCATTGGGGTTTCCCCGCGCAG |
| | | | GTTCGAATCCTGCCGACTACG |
| 1233 | Ser | CGA | GCTGTGATGGCCGAGTGGTTAAGGCGTTGG |
| | | | ACTCGAAATCCAATGGGGTCTCCCCGCGCAG |
| | | | GTTCGAATCCTGCTCACAGCG |
| 1234 | Ser | GCT | GACGAGGNNTGGCCGAGTGGTTAAGGCGAT |
| | | | GGACTGCTAATCCATTGTGCTCTGCACGCGT |
| | | | GGGTTCGAATCCCATCCTCGTCG |
| 1235 | Ser | TGA | GTAGTCGTGGCCGAGTGGTTAAGGCGATGG |
| | | | ACTTGAAATCCATTGGGGTCTCCCCGCGCAG |
| | | | GTTCGAATCCTGCCGGCTACG |
| 1236 | Thr | AGT | GGCTCCGTGGCTTAGCTGGTTAAAGCGCCTG |
| | | | TCTAGTAAACAGGAGATCCTGGGTTCGAATC |
| | | | CCAGCGGGGCCT |
| 1237 | Thr | CGT | GGCNCTGTGGCTNAGTNGGNTAAAGCGCCG |
| | | | GTCTCGTAAACCNGGAGATCNTGGGTTCGA |
| | | | ATCCCANCNGGGCCT |
| 1238 | Thr | TGT | GGCTCCATAGCTCAGNGGGTTAGAGCACTG |
| | | | GTCTTGTAAACCAGGGGTCGCGAGTTCAAAT |
| | | | CTCGCTGGGGCCT |
| 1239 | Trp | CCA | GACCTCGTGGCGCAACGGTAGCGCGTCTGA |
| | | | CTCCAGATCAGAAGGTTGCGTGTTCAAATCA |
| | | | CGTCGGGGTCA |
| 1240 | Tyr | GTA | CCTTCGATAGCTCAGCTGGTAGAGCGGAGG |
| | | | ACTGTAGATCCTTAGGTCGCTGGTTCGATTC |
| | | | CGGCTCGAAGGA |
| 1241 | Val | AAC | GTTTCCGTAGTGTAGTGGTTATCACGTTCGC |
| | | | CTAACACGCGAAAGGTCCCCGGTTCGAAAC |
| | | | CGGGCGGAAACA |
| 1242 | Val | CAC | GTTTCCGTAGTGTAGTGGTTATCACGTTCGC |
| | | | CTCACACGCGAAAGGTCCCCGGTTCGAAAC |
| | | | CGGGCGGAAACA |
| 1243 | Val | TAC | GGTTCCATAGTGTAGTGGTTATCACGTCTGC |
| | | | TTTACACGCAGAAGGTCCTGGGTTCGAGCCC |
| | | | CAGTGGAACCA |
| 1244 | iMet | CAT | AGCAGAGTGGCGCAGCGGAAGCGTGCTGGG |
| | | | CCCATAACCCAGAGGTCGATGGATCGAAAC |
| | | | CATCCTCTGCTA |

TABLE 10B
Consensus sequence computationally generated for each isodecoder by aligning
members of the isodecoder family

| SEQ ID | Amino | | |
|---|---|---|---|
| NO | Acid | Anticodon | Consensus sequence |
| 1245 | Ala | AGC | GGGGAATTAGCTCAAGTGGTAGAGCGCTTGC |
| | | | TTAGCATGCAAGAGGTAGTGGGATCGATGCC |
| | | | CACATTCTCCANNN |
| 1246 | Ala | CGC | GGGGATGTAGCTCAGTGGTAGAGCGCATGCT |
| | | | TCGCATGTATGAGGTCCCGGGTTCGATCCCC |
| | | | GGCATCTCCANNN |
| 1247 | Ala | TGC | GGGGGTGTAGCTCAGTGGTAGAGCGCATGCT |
| | | | TTGCATGTATGAGGCCCCGGGTTCGATCCCC |
| | | | GGCACCTCCANNN |
| 1248 | Arg | ACG | GGGCCAGTGGCGCAATGGATAACGCGTCTGA |
| | | | CTACGGATCAGAAGATTCCAGGTTCGACTCC |
| | | | TGGCTGGCTCGNNN |
| 1249 | Arg | CCG | GGCCGCGTGGCCTAATGGATAAGGCGTCTGA |
| | | | TTCCGGATCAGAAGATTGAGGGTTCGAGTCC |
| | | | CTTCGTGGTCGNNN |
| 1250 | Arg | CCT | GCCCCAGTGGCCTAATGGATAAGGCACTGGC |
| | | | CTCCTAAGCCAGGGATTGTGGGTTCGAGTCC |
| | | | CACCTGGGGTANNN |
| 1251 | Arg | TCG | GACCGCGTGGCCTAATGGATAAGGCGTCTGA |
| | | | CTTCGGATCAGAAGATTGAGGGTTCGAGTCC |
| | | | CTCCGTGGTCGNNN |
| 1252 | Arg | TCT | GGCTCTGTGGCGCAATGGATNAGCGCATTGG |
| | | | ACTTCTAATTCAAAGGTTGCGGGTTCGAGTC |
| | | | CCNCCAGAGTCGNNN |
| 1253 | Asn | GTT | GTCTCTGTGGCGCAATCGGTTAGCGCGTTCG |
| | | | GCTGTTAACCGNAAAGGTTGGTGGTTCGAGC |
| | | | CCACCCAGGGACGNNN |
| 1254 | Asp | GTC | TCCTCGTTAGTATAGTGGTGAGTATCCCCGCC |
| | | | TGTCACGCGGGAGACCGGGGTTCGATTCCCC |
| | | | GACGGGGAGNNN |
| 1255 | Cys | GCA | GGGGGTATAGCTCAGNGGGTAGAGCATTTGA |
| | | | CTGCAGATCAAGAGGTCCCCGGTTCAAATCC |
| | | | GGGTGCCCCCTNNN |
| 1256 | Gln | CTG | GGTTCCATGGTGTAATGGTNAGCACTCTGGA |
| | | | CTCTGAATCCAGCGATCCGAGTTCAAGTCTC |
| | | | GGTGGAACCTNNN |
| 1257 | Gln | TTG | GGTCCCATGGTGTAATGGTTAGCACTCTGGA |
| | | | CTTTGAATCCAGCGATCCGAGTTCAAATCTC |
| | | | GGTGGGACCTNNN |
| 1258 | Glu | CTC | TCCCTGGTGGTCTAGTGGTTAGGATTCGGCG |
| | | | CTCTCACCGCCGCGGCCCGGGTTCGATTCCC |
| | | | GGTCAGGGAANNN |
| 1259 | Glu | TTC | TCCCTGGTGGTCTAGTGGCTAGGATTCGGCG |
| | | | CTTTCACCGCNGCGGCCCGGGTTCGATTCCC |
| | | | GGTCAGGGAANNN |
| 1260 | Gly | CCC | GCATTGGTGGTTCAGTGGTAGAATTCTCGCCT |
| | | | CCCACGCNGGAGACCCGGGTTCGATTCCCGG |
| | | | CCAATGCANNN |
| 1261 | Gly | GCC | GCATTGGTGGTTCAGTGGTAGAATTCTCGCCT |
| | | | GCCACGCGGGAGGCCCGGGTTCGATTCCCGG |
| | | | CCAATGCANNN |
| 1262 | Gly | TCC | GCGTTGGTGGTATAGTGGTGAGCATAGCTGC |
| | | | CTTCCAAGCAGTTGACCCGGGTTCGATTCCC |
| | | | GGCCAACGCANNN |
| 1263 | Ile | AAT | GGCCGGTTAGCTCAGTTGGTTAGAGCGTGGT |
| | | | GCTAATAACGCCAAGGTCGCGGGTTCGATCC |
| | | | CCGTACGGGCCANNN |
| 1264 | Ile | TAT | GCTCCAGTGGCGCAATCGGTTAGCGCGCGGT |
| | | | ACTTATAATGCCGAGGTTGTGAGTTCGAGCC |
| | | | TCACCTGGAGCANNN |
| 1265 | Leu | AAG | GGTAGCGTGGCCGAGCGGTCTAAGGCGCTGG |
| | | | ATTAAGGCTCCAGTCTCTTCGGGGGCGTGGG |
| | | | TTCGAATCCCACCGCTGCCANNN |
| 1266 | Leu | CAA | GTCAGGATGGCCGAGTGGTCNTAAGGCGCCA |
| | | | GACTCAAGTTCTGGTCTCCGNATGGAGGCGT |
| | | | GGGTTCGAATCCCACTTCTGACANNN |
| 1267 | Leu | CAG | GTCAGGATGGCCGAGCGGTCTAAGGCGCTGC |
| | | | GTTCAGGTCGCAGTCTCCCCTGGAGGCGTGG |
| | | | GTTCGAATCCCACTCCTGACANNN |
| 1268 | Leu | TAA | ACCAGGATGGCCGAGTGGTTAAGGCGTTGGA |
| | | | CTTAAGATCCAATGGACAGATGTCCGCGTGG |
| | | | GTTCGAACCCCACTCCTGGTANNN |
| 1269 | Leu | TAG | GGTAGCGTGGCCGAGCGGTCTAAGGCGCTGG |
| | | | ATTTAGGCTCCAGTCTCTTCGGNGGCGTGGG |
| | | | TTCGAATCCCACCGCTGCCANNN |
| 1270 | Lys | CTT | GCCCGGCTAGCTCAGTCGGTAGAGCATGAGA |
| | | | CTCTTAATCTCAGGGTCGTGGGTTCGAGCCCC |
| | | | ACGTTGGGCGNNNNNN |
| 1271 | Lys | TTT | GCCTGGATAGCTCAGTCGGTAGAGCATCAGA |
| | | | CTTTTAATCTGAGGGTCCAGGGTTCAAGTCCC |
| | | | TGTTCAGGCGNNN |
| 1272 | Met | CAT | GCCCTCTTAGCGCAGTNGGCAGCGCGTCAGT |
| | | | CTCATAATCTGAAGGTCCTGAGTTCGAGCCT |
| | | | CAGAGAGGGCANNN |
| 1273 | Phe | GAA | GCCGAAATAGCTCAGTTGGGAGAGCGTTAGA |
| | | | CTGAAGATCNTAAAGGTCCCTGGTTCAATCC |
| | | | CGGGTTTCGGCANNN |
| 1274 | Pro | AGG | GGCTCGTTGGTCTAGGGGTATGATTCTCGCTT |
| | | | AGGATGCGAGAGGTCCCGGGTTCAAATCCCG |
| | | | GACGAGCCCNNN |
| 1275 | Pro | CGG | GGCTCGTTGGTCTAGGGGTATGATTCTCGCTT |
| | | | CGGGTGCGAGAGGTCCCGGGTTCAAATCCCG |
| | | | GACGAGCCCNNN |
| 1276 | Pro | TGG | GGCTCGTTGGTCTAGGGGTATGATTCTCGCTT |
| | | | TGGGTGCGAGAGGTCCCGGGTTCAAATCCCG |
| | | | GACGAGCCCNNN |
| 1277 | Ser | AGA | GTAGTCGTGGCCGAGTGGTTAAGGCGATGGA |
| | | | CTAGAAATCCATTGGGGTTTCCCCGCGCAGG |
| | | | TTCGAATCCTGCCGACTACGNNN |
| 1278 | Ser | CGA | GCTGTGATGGCCGAGTGGTTAAGGCGTTGGA |
| | | | CTCGAAATCCAATGGGGTCTCCCCGCGCAGG |
| | | | TTCGAATCCTGCTCACAGCGNNN |
| 1279 | Ser | GCT | GACGAGGNNTGGCCGAGTGGTTAAGGCGAT |
| | | | GGACTGCTAATCCATTGTGCTCTGCACGCGT |
| | | | GGGTTCGAATCCCATCCTCGTCGNNN |
| 1280 | Ser | TGA | GTAGTCGTGGCCGAGTGGTTAAGGCGATGGA |
| | | | CTTGAAATCCATTGGGGTCTCCCCGCGCAGG |
| | | | TTCGAATCCTGCCGGCTACGNNN |
| 1281 | Thr | AGT | GGCTCCGTGGCTTAGCTGGTTAAAGCGCCTG |
| | | | TCTAGTAAACAGGAGATCCTGGGTTCGAATC |
| | | | CCAGCGGGGCCTNNN |
| 1282 | Thr | CGT | GGCNCTGTGGCTNAGTNGGNTAAAGCGCCGG |
| | | | TCTCGTAAACCNGGAGATCNTGGGTTCGAAT |
| | | | CCCANCNGGGCCTNNN |
| 1283 | Thr | TGT | GGCTCCATAGCTCAGNGGGTTAGAGCACTGG |
| | | | TCTTGTAAACCAGGGGTCGCGAGTTCAAATC |
| | | | TCGCTGGGGCCTNNN |
| 1284 | Trp | CCA | GACCTCGTGGCGCAACGGTAGCGCGTCTGAC |
| | | | TCCAGATCAGAAGGTTGCGTGTTCAAATCAC |
| | | | GTCGGGGTCANNN |
| 1285 | Tyr | GTA | CCTTCGATAGCTCAGCTGGTAGAGCGGAGGA |
| | | | CTGTAGATCCTTAGGTCGCTGGTTCGATTCCG |
| | | | GCTCGAAGGANNN |
| 1286 | Val | AAC | GTTTCCGTAGTGTAGTGGTTATCACGTTCGCC |
| | | | TAACACGCGAAAGGTCCCCGGTTCGAAACCG |
| | | | GGCGGAAACANNN |
| 1287 | Val | CAC | GTTTCCGTAGTGTAGTGGTTATCACGTTCGCC |
| | | | TCACACGCGAAAGGTCCCCGGTTCGAAACCG |
| | | | GGCGGAAACANNN |
| 1288 | Val | TAC | GGTTCCATAGTGTAGTGGTTATCACGTCTGCT |
| | | | TTACACGCAGAAGGTCCTGGGTTCGAGCCCC |
| | | | AGTGGAACCANNN |
| 1289 | iMet | CAT | AGCAGAGTGGCGCAGCGGAAGCGTGCTGGG |
| | | | CCCATAACCCAGAGGTCGATGGATCGAAACC |
| | | | ATCCTCTGCTANNN |

TABLE 11
Score values alignment

| | Candidate | Reference | Match |
|---|---|---|---|
| Row | nucleotide | nucleotide | score |
| 1 | A | A | 1 |
| 2 | T | T | 1 |
| 3 | U | T | 1 |
| 4 | C | C | 1 |
| 5 | G | G | 1 |
| 6 | A | N | 0 |
| 7 | T | N | 0 |
| 8 | C | N | 0 |
| 9 | G | N | 0 |
| 10 | N | A | 0 |
| 11 | N | T | 0 |
| 12 | N | C | 0 |
| 13 | N | G | 0 |
| 14 | N | N | 0 |

### Premature Termination Codons (PTC) and ORFs Comprising PTCs

Mutations underlie many diseases. For example, a point mutation in the open reading frame (ORF) of a gene which creates a premature stop codon (PTC) can result in altered expression and/or activity of a polypeptide encoded by the gene. Table 15 provides single mutations in codons encoding amino acids which can result in a stop codon. In an embodiment, a PTC disclosed herein comprises a mutation disclosed in Table 15.

In an embodiment, the codon having the first sequence or the PTC comprises a mutation disclosed in Table 15. In an embodiment, the non-mutated, e.g., wildtype, codon sequence of the codon having the first sequence or the PTC is an original codon sequence provided in Table 15 and the amino acid corresponding to the non-mutated codon is an original AA provided in Table 15.

In an embodiment, the TREM, TREM core fragment or TREM fragment recognizes a stop codon and mediates incorporation of the original AA provided in Table 15 at the position of the stop codon. In an embodiment, the TREM, TREM core fragment or TREM fragment recognizes a stop codon and mediates incorporation of an amino acid belonging to the same group as the original AA, e.g., as provided in Table 16. Other genetic abnormalities, such as insertions and/or deletions can also result in a PTC in an ORF.

TABLE 15
Select amino acids and stop codons

| Original | Original | One mutation to stop |
|---|---|---|
| AA | codon | codon |
| TRP | UGG | UGA |
| TYR | UAU | UAA |
| | UAC | UAG |
| CYS | UGU | UGA |
| | UGC | UGA |
| GLU | GAA | UAA |
| | GAG | UAG |
| LYS | AAA | UAA |
| | AAG | UAG |
| GLN | CAA | UAA |
| | CAG | UAG |
| SER | UCA | UGA |
| | UCG | UAG |
| LEU | UUA | UAA OR UGA |
| | UUG | UAG |
| ARG | CGA | UGA |
| GLY | GGA | UGA |

TABLE 16
Amino acids and amino acid groupings

| Group | Amino acid |
|---|---|
| Nonpolar, aliphatic R group | leucine |
| | methionine |
| | isoleucine |
| | glycine |
| | alanine |
| | valine |
| Polar, uncharged R group | serine |
| | threonine |
| | cysteine |
| | proline |
| | asparagine |
| | glutamine |
| positively charged r group | lysine |
| | arginine |
| | histidine |
| Negatively charged R group | aspartate |
| | glutamate |
| Nonpolar, aromatic R group | phenylalanine |
| | tyrosine |
| | tryptophan |

Disclosed herein, inter alia, are endogenous ORFs comprising a codon having a first sequence, e.g., a mutation, e.g., a PTC. An ORF having a PTC, e.g., as described herein, can be present, or part of in any gene. As an example, the ORF can be present or be part of any gene in the human genome.

In an embodiment, a PTC disclosed herein is present in a gene disclosed in any one of Tables 17, 18, or 20. Exemplary genes having OR-Fs comprising a PTC are provided in Table 3.

TABLE 17
Exemplary genes with ORFs having a PTC

| | | | | |
|---|---|---|---|---|
| A2ML1 | ARFGEF1 | CACNA1G | CNOT1 | DLG4 |
| AARS1 | ARFGEF2 | CACNA1S | COG1 | DLL1 |
| AARS2 | ARHGAP21 | CACNA2D1 | COL11A2 | DNA2 |
| ABCA13 | ARHGEF9 | CACNA2D2 | COL13A1 | DNM1L |
| ABCB11 | ARMC4 | CACNB2, NSUN6 | COL4A1 | DNMT1 |
| ABCG5 | ARV1 | CAD | COL4A2 | DNMT3A |
| ABHD5 | ARX | CAMTA1 | COL4A4 | DNMT3B |
| ACAD8 | ASCC3 | CARS2 | COL9A1 | DPH1 |
| ACADL | ASH1L | CCDC140 | COQ4 | DPYD-AS1 |
| ACSF3 | ASPH | CCDC8 | COQ6 | DSEL |
| ACTA2 | ASXL2 | CCM2 | COX14 | DSPP |
| ACTC1, | ATAD3A | CD40LG | CPE | DUOXA1 |
| ACTN2 | ATP2A2 | CDAN1 | CPEB1-AS1 | DUOXA2 |
| ACVR1 | ATP6V1B1 | CDH15 | CREBBP | DVL1 |
| ADAR | ATP8A2 | CDK11A | CRELD1 | EARS2 |
| ADAT3 | AUH | CEBPA | CSNK2A1 | EBF3 |
| ADCY5 | AUTS2 | CELF5 | CSNK2B | EBP |
| ADIPOQ | AVPR2 | CELSR2 | CSTA | EDAR |
| ADIPOQ-AS1 | B3GLCT | CEP135 | CTNND2 | EFHC1 |
| ADIPOR1 | B4GAT1 | CEP164 | CTSA | EFNB1 |
| AFF2 | BCAP31 | CEP83 | CTSC | EFTUD2 |
| ALG11 | BCL11A | CETP | CUL3 | EIF2B5 |
| ALG14 | BCL11B | CFI | CYLD | ELANE |
| ALG6 | BCORL1 | CHAMP1 | CYP11A1 | EMC1 |
| ALOXE3 | BEND2 | CHAT | DAG1 | EMC1-AS1 |
| AMER1 | BGN | CHD1 | DARS2 | ENO3 |
| AMH | BMP4 | CHD4 | DCX | ENTPD5 |
| AMMECR1 | BRD4 | CHD8 | DDHD2 | EP300 |
| AMN | BRPF1 | CHRM2 | DDR2 | EPM2A |
| ANK2 | BRSK2 | CHRNB1 | DEAF1 | ERLIN2 |
| ANK3 | BUB1B | CIC | DENND5A | EVC |
| ANKS6 | BUB1B-PAK6 | CLCN7 | DGAT1 | EZH2 |
| ANOS1 | C8G | CLTC | DHFR | FAM111A |
| AP1S1 | CACNA1E | CNGA3 | DIAPH3 | FAM126A |
| AP3B2 | CACNA1F | CNKSR2 | DISP1 | FAN1 |
| FANCD2 | GJB6 | ISLR2 | LOC110673972 | MTR |
| FANCE | GJC2 | ITGA3 | LOC112997540 | MYCBP2 |
| FASTKD2 | GK | ITGA8 | LOC113788297 | MYCNOS |
| FAT1 | GLI2 | ITGB6 | LOC349160 | MYH7B |
| FBN2 | GNAI1 | JMJD1C | LONP1 | MYL3 |
| FBP1 | GNB1L | KANK1 | LORICRIN | MYOM1 |
| FDXR | GNE | KANSL1 | LPIN1 | MYPN |
| FGA | GNRH1 | KBTBD13 | LPIN2 | MYT1L |
| FGD1 | GNS | KCNA2 | LRP2 | NAA15 |
| FGF10 | GPAA1 | KCNB1 | LRRTM4 | NAGA |
| FGFR1 | GPD1L | KCND2 | MAB21L2 | NARS2 |
| FGFR2 | GRIN2A | KCNE3 | MAF | NAXE |
| FIBP | GTPBP3 | KCNMA1 | MARS1 | NCAPH2 |
| FLAD1 | HACE1 | KCNQ5 | MARS2 | NCF2 |
| FLG-AS1 | HADH | KCTD7 | MASP1 | NDP |
| FLVCR1 | HADHB | KIDINS220 | MBOAT7 | NDP-AS1 |
| FLVCR2 | HDAC4 | KIF21B | MCM3AP | NDRG1 |
| FMN2 | HERC1 | KIF6 | MCM3AP-AS1 | NDUFA2, TMCO6 |
| FOXA2 | HESX1 | KIT | MED13 | NDUFAF1 |
| FOXC1 | HIBCH | KLHL40 | MED17 | NDUFAF5 |
| FOXC2 | HNF4A | KLHL41 | MEGF10 | NDUFAF6 |
| FOXC2-AS1 | HNRNPH2 | KNL1 | MET | NDUFAF7 |
| FOXP2 | HPRT1 | KRAS | MGAT2 | NDUFS1 |
| FREM1 | HRG | LAMB1 | MIB1 | NDUFS3 |
| FRYL | HUWE1 | LAMB2 | MICU1 | NEFH |
| FTL | IARS1 | LAMC3 | MIR302CHG | NEK8 |
| FUS | IBA57 | LARP7 | MIR5004 | NEXMIF |
| GABRG2 | IDH2 | LARS1 | MIR6501 | NFIA |
| GAN | IFNAR1 | LCT | MITD1 | NFKB1 |
| GATA2 | IFT122 | LEMD3 | MMP13 | NHEJ1 |
| GATA4 | IFT80 | LGI4 | MMP21 | NICN1 |
| GATAD1 | IGF2 | LIAS | MNX1 | NID1 |
| GDF5 | ILDR1 | LINS1 | MNX1-AS2 | NKX2-1 |
| GDF5-AS1 | ILK, TAF10 | LIPC | MPDU1 | NLRP1 |
| GFM1 | INF2 | LIPT1 | MRPS22 | NLRP3 |
| GH-LCR | INS-IGF2 | LOC101448202 | MSL3 | NOD2 |
| GHRHR | INSR | LOC106804612, HBA2 | MSRB3 | NONO |
| GHSR | IRAK1BP1 | LOC107303338 | MT-ND2 | NOTCH3 |
| GJA1 | IRAK3 | RARS2 | SETD1B | SPTLC1 |
| NPHP4 | PLEKHG5 | RAX | SETD2 | SRD5A3 |
| NPR2 | PLEKHM2 | RBM10 | SETX | SRPX2 |
| NR2F2 | PLK4 | RELN | SFTA3 | SSBP2 |
| NR5A1 | PLPBP | RERE | SHANK2 | ST3GAL3 |
| NRL, PCK2 | PNKD | RFT1 | SHH | ST3GAL5 |
| NRXN1 | PNPLA1 | RMND1 | SIN3A | STAMBP |
| NT5DC1 | POC1A | RNASEH1 | SIX3 | STAT3 |
| NTRK2 | POLG2, MILR1 | RNF17 | SKI | STIL |
| NUBPL | POMGNT2 | ROR2 | SLC13A5 | STX11 |
| NUS1 | PPM1D | RP2 | SLC16A1 | STX1B |
| OCRL | PPP3CA | RPL11 | SLC16A2 | SUCLA2 |
| OPTN | PRDM1 | RPS19 | SLC17A8 | SYN1 |
| P4HA1 | PRDM12 | RRM2B | SLC18A3 | SYN2 |
| PAK6 | PREPL | RS1 | SLC20A2 | SYNJ1 |
| PBX1 | PRICKLE1 | RUNX2 | SLC25A4 | TAB2 |
| PCARE | PRKAG2 | RXYLT1 | SLC25A46 | TACR3 |
| PCDH12 | PROS1 | S100PBP | SLC2A1 | TBCD |
| PDCD10 | PRPF31 | SALL4 | SLC39A8 | TBL1XR1 |
| PDE11A-AS1 | PRPF8 | SAMD9 | SLC52A2 | TBX1 |
| PDE4D | PRPS1 | SAR1B | SLC6A3 | TBX18 |
| PDE6A | PRSS1, TRB | SASH3 | SLC6A8 | TCIRG1 |
| PDHX | PSAT1 | SBF2 | SLC6A9 | TELO2 |
| PDLIM3 | PSMD12 | SBF2-AS1 | SLC7A9 | TFAP2A |
| PDP1 | PSTPIP1 | SCAMP4 | SMAD2 | TFG |
| PDSS1 | PTCHD1 | SCLT1 | SMAD9 | TGIF1 |
| PEX5 | PTF1A | SCN10A | SMARCA2 | THAP1 |
| PHF21A | PTPN23 | SCN11A | SMC3 | TINF2 |
| PHF8 | PTPRQ | SCN1B | SMOC2 | TLK2 |
| PHKA2 | PTRH2 | SCN3A | SNTA1 | TMEM43 |
| PHKB | PUS1 | SCN4A | SNX14 | TMIE |
| PIEZO1 | PYCR2 | SCN4B | SNX22 | TMPO |
| PIGG | QARS1 | SCN8A | SOCS1 | TMPRSS15 |
| PIGL | RAB3GAP1, ZRANB3 | SCO2 | SOX11 | TMTC3 |
| PIGM | RAB3GAP2 | SCYL1 | SOX17 | TNFAIP3 |
| PIGP | RAC1 | SEMA4A | SPATA5 | TNFRSF11A |
| PIK3CA | RAF1 | SEPTIN9 | SPATA7 | TOE1 |
| PIN4, ERCC6L | RAG1 | SET | SPRED1 | TOR1AIP1 |
| PLAT | RARB | SETD1A | SPTBN2 | TPK1 |
| TPM1 | UTP14C | ZEB1 | | |
| TRAPPC9 | VPS53 | ZFHX4 | | |
| TRIM37 | WDR19 | ZFPM2 | | |
| TRIM59-IFT80 | WDR26 | ZFPM2-AS1 | | |
| TRIO | WDR62 | ZIC1 | | |
| TRIP12 | WDR81 | ZIC2 | | |
| TRIP4 | WNT1 | ZMYND11 | | |
| TRPM1 | WNT10A | ZNF335 | | |
| TSEN54 | WRAP53 | ZNF423 | | |
| TUBA4A | WWOX | ZNF469 | | |
| TUBGCP4 | YARS1 | | | |
| TUBGCP6 | YARS2 | | | |
| TWNK | YY1 | | | |
| TXNRD2 | ZAP70 | | | |
| UBA2 | ZBTB20 | | | |
| UBA5 | ZBTB24 | | | |
| UMOD | ZC4H2 | | | |
| UNC5B | ZDHHC9 | | | |

Additional exemplary genes containing a PTC include FVIII, FIX, CFTR, MeCP2, NAGLU, DMD, GAA, RP1, RP2, ABCA4, PCDH15, REP1, GLA, MUT, TP53, and ATM. In an embodiment, the PTC is present within the FVIII gene and comprises an R mutation, e.g., an R2228X mutation. In an embodiment, the PTC is present within the FIX gene and comprises an R mutation, e.g., an R29X mutation, an R116X mutation, an R248X mutation, an R252X mutation, an R333X mutation, and/or an R338X mutation. In an embodiment, the PTC is present within the CFTR gene and comprises an R mutation, e.g., an R553X mutation. In an embodiment, the PTC is present within the MeCP2 gene and comprises an R mutation, e.g., an R168X mutation. In an embodiment, the PTC is present within the NAGLU gene and comprises an R mutation, e.g., an R626X mutation. In an embodiment, t the PTC is present within the DMD gene and comprises an R mutation, e.g., an R3881X mutation. In an embodiment, the PTC is present within the GAA gene and comprises an R mutation, e.g., an R854X mutation. In an embodiment, the PTC is present within the RP1 gene and comprises an R mutation, e.g., an R667X mutation. In an embodiment, the PTC is present within the RP2 gene and comprises an R mutation, e.g., an R120X mutation. In an embodiment, the PTC is present within the ABCA4 gene and comprises an R mutation, e.g., an R2030X mutation. In an embodiment, the PTC is present within the PCD gene and comprises an R mutation, e.g., an R245X mutation. In an embodiment, the PTC is present within the REP1 gene and comprises an R mutation, e.g., an R270X mutation. In an embodiment, the PTC is a mutation in the GLA gene, e.g., an R220X mutation and/or an R227X mutation. In an embodiment, the PTC is present within the MUT gene and comprises an R mutation, e.g., an R228X mutation, an R403X mutation, an R467X mutation, and/or an R727X mutation. In an embodiment, the PTC is present within the TP53 gene and comprises an R mutation, e.g., an R578X mutation. In an embodiment the PTC is present within the ATM gene and comprises an R mutation, e.g., an R35X mutation.

Diseases or Disorders Associated with a PTC

A TREM composition disclosed herein can be used treat a disorder or disease associated with a PTC, e.g., as described herein. Exemplary diseases or disorders associated with a PTC are listed in Tables 18, 19, and 20.

In an embodiment, the subject has a disease or disorder provided in any one of Tables 4-6. In an embodiment, the cell is associated with, e.g., is obtained from a subject who has, a disorder or disease listed in any one of Tables 18-20.

For example, the disorder or disease can be chosen from the left column of Table 18. As another example, the disorder or disease is chosen from the left column of Table 18 and, in embodiments the PTC is in a gene chosen from the right column of Table 18, e.g., any one of the genes provided in the right column of Table 18. In some embodiments, the PTC is in a gene corresponding to the disorder or disease provided in the left column of Table 18. As a further non-limiting example, the PTC can be at a position provided in Table 18.

As another example, the disorder or symptom is chosen from a disorder or disease provided in Table 19.

As yet another example, the disorder or symptom is chosen from a disorder or disease provided in Table 20. In an embodiment, the disorder or symptom is chosen from a disorder or disease provided in Table 20 and, in embodiments, the PTC is in any gene provided in Table 20. In an embodiment, the disorder or symptom is chosen from a disorder or disease provided in Table 20 and the PTC is in a corresponding gene provided in Table 20, e.g., a gene corresponding to the disease or disorder. In an embodiment, the disorder or symptom is chosen from a disorder or disease provided in Table 20 and the PTC is not in a gene provided in Table 20.

In an embodiment of any of the methods disclosed herein, the PTC is at any position within the ORF of the gene, e.g., upstream of the naturally occurring stop codon.

TABLE 18
Exemplary diseases or disorders

| | |
|---|---|
| Disease/disorder or protein | Exemplary Point Mutation |

G to A point mutations

| | |
|---|---|
| Dihydropyrimidine dehydrogenase | NM 000110.3(DPYD): c.1905 + 1G > A |
| deficiency | |
| Noonan syndrome | NM 005633.3(SOS1): c.2536G > A |
| | (p.Glu846Lys) |
| Lynch syndrome | NM 000251.2(MSH2): c.212 − 1G > A |
| Breast-ovarian cancer, familial 1 | NM 007294.3(BRCA1): c.963G > A |
| | (p.Trp321Ter) |
| Cystic fibrosis | NM 000492.3(CFTR): c.57G > A (p.Trpl9Ter) |
| Anemia, due to G6PD deficiency | NM 000402.4(G6PD): c.292G > A |
| | (p.Val98Met) |
| AVPR2 | NM 000054.4(AVPR2): c.878G > A |
| Nephrogenic diabetes insipidus, X-linked | (p.Trp293Ter) |
| FANCC | NM 000054.4(AVPR2): c.878G > A |
| Fanconi anemia, complementation group C | (p.Trp293Ter) |
| FANCC | NM 000136.2(FANCC): c.1517G > A |
| Fanconi anemia, complementation group C | (p.Trp506Ter) |
| IL2RG | NM 000206.2(IL2RG): c.710G > A |
| X-linked severe combined | (p.Trp237Ter) |
| immunodeficiency | |
| F8 Hereditary factor VIII deficiency | NM 000132.3(F8): c.3144G > A |
| disease | (p.Trpl048Ter) |
| LDLR | NM 000527.4(LDLR): c.1449G > A |
| Familial hypercholesterolemia | (p.Trp483Ter) |
| CBS | NM 000071.2(CBS): c.162G > A |
| Homocystinuria due to CBS deficiency | (p.Trp54Ter) |
| HBB | NM 000518.4(HBB): c.114G > A |
| betaThalassemia | (p.Trp38Ter) |
| ALDOB | NM 000035.3(ALDOB): c.888G > A |
| Hereditary fmctosuria | (p.Trp296Ter) |
| DMD | NM 004006.2(DMD): c.3747G > A |
| Duchenne muscular dystrophy | (p.Trpl249Ter) |
| SMAD4 | NM 005359.5(SMAD4): c.906G > A |
| Juvenile polyposis syndrome | (p.Trp302Ter) |
| BRCA2 | NM 000059.3(BRCA2): c.582G > A |
| Familial cancer ofbreastlBreast-ovarian | (p.Trpl94Ter) |
| cancer, familial 2 | |
| GRIN2A | NM 000833.4(GRIN2A): c.3813G > A |
| Epilepsy, focal, with speech disorder and | (p.Trpl271Ter) |
| with or without mental retardation | |
| SCN9A | NM 002977.3(SCN9A): c.2691G > A |
| Indifference to pain, congenital, | (p.Trp897Ter) |
| autosomal recessive | |
| TARDBP | NM 007375.3(TARDBP): c.943G > A |
| Amyotrophic lateral sclerosis type 10 | (p.Ala315Thr) |
| CFTR | NM 000492.3(CFTR): c.3846G > A |
| Cystic fibrosislHereditary | (p.Trpl282Ter) |
| pancreatitislnot providedlataluren | |
| response - Efficacy | |
| UBE3A | NM 130838. l(UBE3A): c.2304G > A |
| Angelman syndrome | (p.Trp768Ter) |
| SMPD1 | NM 000543.4(SMPD1): c.168G > A |
| Niemann-Pick disease, type A | (p.Trp56Ter) |
| USH2A | NM 206933.2(USH2A): c.9390G > A |
| Usher syndrome, type 2A | (p.Trp3130Ter) |
| MENl | NM 130799.2(MEN1): c.1269G > A |
| Hereditary cancer-predisposing syndrome | (p.Trp423Ter) |
| C8orf37 | NM 177965.3(C8orf37): c.555G > A |
| Retinitis pigmentosa 64 | (p.Trpl85Ter) |
| MLHl | NM 000249.3(MLH1): c.1998G > A |
| Lynch syndrome | (p.Trp666Ter) |
| TSC2 | NM 000548.4(TSC2): c.2108G > A |
| Tuberous sclerosis 21Tuberous | (p.Trp703Ter) |
| sclerosis syndrome 46 | |
| NFl | NM 000267.3(NF1): c.7044G > A |
| Neurofibromatosis, type 1 | (p.Trp2348Ter) |
| MSH6 | NM 000179.2(MSH6): c.3020G > A |
| Lynch syndrome | (p.Trpl007Ter) |
| SMNl | NM 000344.3(SMN1): c.305G > A |
| Spinal muscular atrophy, type III | (p.Trpl02Ter) |
| Kugelberg- Welander disease | |
| SH3TC2 | NM 024577.3(SH3TC2): c.920G > A |
| Charcot-Marie-Tooth disease, type 4C | (p.Trp307Ter) |
| DNAH5 | NM 001369.2(DNAH5): c.8465G > A |
| Primary ciliary dyskinesia | (p.Trp2822Ter) |
| MECP2 | NM 004992.3(MECP2): c.311G > A |
| Rett syndrome | (p.Trpl04Ter) |
| ADGRVl | NM 032119.3(ADGRV1): c.7406G > A |
| Usher syndrome, type 2C | (p.Trp2469Ter) |
| AHil | NM 017651.4(AHI1): c.2174G > A |
| Joubert syndrome 3 | (p.Trp725Ter) |
| PRKN | NM 004562.2(PRKN): c.1358G > A |
| Parkinson disease 2 | (p.Trp453Ter) |
| COL3Al | NM 000090.3(COL3Al): c.3833G > A |
| Ehlers-Danlos syndrome, type 4 | (p.Trpl278Ter) |
| BRCAl | NM 007294.3(BRCA1): c.5511G > A |
| Familial cancer ofbreastlBreast-ovarian | (p.Trpl837Ter) |
| cancer, familial 1 | |
| MYBPC3 | NM 000256.3(MYBPC3): c.3293G > A |
| Primary familial hypertrophic | (p.Trpl098Ter) |
| cardiomyopathy | |
| APC | NM 000038.5(APC): c.1262G > A |
| Familial adenomatous polyposis 1 | (p.Trp421Ter) |
| BMPR2 | NM 001204.6(BMPR2): c.893G > A |
| Primary pulmonary hypertension | (p.W298*) |

T to C point mutations

| | |
|---|---|
| Wilson disease | NM_000053.3(ATP7B): c.3443T > C (p.Ile l l |
| | 48Thr) |
| Leukodystrophy, hypomyelinating, 2 | NM_020435.3(GJC2): c.857T > C |
| | (p.Met286Thr) |
| Alport syndrome, X-linked recessive | NM_000495.4(COL4A5): c.438 + 2T > C |
| Leigh disease | NC 012920.1: m.9478T > C |
| Gaucher disease, type 1 | NM_001005741.2(GBA): c.751T > C |
| | (p.Tyr251His) |
| Renal dysplasia, retinal pigmentary | NM_0l4714.3(IFT140): c.4078T > C |
| dystrophy, cerebellar ataxia and skeletal | (p.Cysl360Arg) |
| dysplasia | |
| Marfan syndrome | NM_000138.4(FBN1): c.3793T > C |
| | (p.Cysl265Arg) |
| Deficiency of UDPglucose-hexose-1- | NM_000155.3(GALT): c.482T > C |
| phosphate uridylyltransferase | (p.Leul61Pro) |
| Familial hypercholesterolemia | NM_000527.4(LDLR): c.694 + 2T > C |
| Episodic pain syndrome, familial, 3 | NM_001287223.l(SCN11A): c.1142T > C |
| | (p.Ile381Thr) |
| Navajo neurohepatopathy | NM_002437.4(MPV17): c.186 + 2T > C |
| Congenital muscular dystrophy, LMNA- | NM_l 70707.3(LMNA): c.l139T > C |
| related | (p.Leu380Ser) |
| Hereditary factor VIII deficiency disease | NM_000132.3(F8): c.5372T > C |
| | (p.Metl791Thr) |
| Insulin-dependent diabetes mellitus | NM_0l4009.3(FOXP3): c.970T > C |
| secretory diarrhea syndrome | (p.Phe324Leu) |
| Hereditary factor IX deficiency disease | NM_000133.3(F9): c.1328T > C (p.Ile443Thr) |
| Familial cancer of breast, Breast-ovarian | NM_000059.3(BRCA2): c.316 + 2T > C |
| cancer, familial 2, Hereditary cancer | |
| predisposing syndrome | |
| Cardiac arrhythmia | NM_000238.3(KCNH2): c.1945 + 6T > C |
| Tangier disease | NM_005502.3(ABCA1): c.4429T > C |
| | (p.Cysl477Arg) |
| Dilated cardiomyopathy lAA | NM_001103.3(ACTN2): c.683T > C |
| | (p.Met228Thr) |
| Mental retardation 3, X-linked | NM_005334.2(HCFC1): c.−970T > C |
| Limb-girdle muscular dystrophy, type 2B | NM_003494.3(DYSF): c.1284 + 2T > C |
| Macular dystrophy, vitelliform, 5 | NM_0l6247.3(IMPG2): c.370T > C |
| | (p.Phel24Leu) |
| Retinitis pigmentosa | NM_000322.4(PRPH2): c.736T > C |
| | (p.Trp246Arg) |

TABLE 19
Additional exemplary disorders

| | |
|---|---|
| 5q-syndrome | Holt-Oram syndrome |
| Adams-Oliver syndrome-1 | Hypoparathyroidism |
| Adams-Oliver syndrome-3 | Intellectual disability |
| Adams-Oliver syndrome-5 | Kleefstra syndrome 1 |
| Adams-Oliver syndrome-6 | Klippel-Trenaunay syndrome (AAGF-related) |
| Alagille syndrome-1 | Marfan syndrome |
| Autoimmune lymphoproliferative syndrome | Mental retardation |
| type IA | |
| Autoimmune lymphoproliferative syndrome | Mental retardation |
| type V | |
| Autosomal dominant deafness-2A | Mental retardation |
| Brain malformations with or without urinary | Mental retardation and distinctive facial |
| tract defects (BRMUTD) | features with or without cardiac defects |
| | (MRFACD) |
| Carney complex type 1 | Nail-patella syndrome (NPS) |
| CHARGE syndrome | Obesity |
| Cleidocranial dysplasia | Phelan-McDermid syndrome |
| Currarino syndrome | Pitt-Hopkins syndrome |
| Denys-Drash syndrome/Frasier syndrome | Primary pulmonary hypertension-1 |
| Developmental delay | Rett syndrome (congenital variant) |
| DiGeorge syndrome (TBXl-associated) | Sensorineural deafness |
| Dravet syndrome | Smith-Magenis syndrome (RAI1-associated) |
| Duane-radial ray syndrome | Sotos syndrome-1 |
| Ehlers-Danlos syndrome (classic-like) | Sotos syndrome-2 |
| Ehlers-Danlos syndrome (vascular type) | Stickler syndrome type I |
| Feingold syndrome-1 | Supravalvular aortic stenosis |
| Feri-Weill dyschondrosteosis | SYNGAP1-related intellectual disability |
| Frontotemporal lobar degeneration with | Treacher Collins syndrome |
| TDP43 inclusions (FTFD-TDP) | |
| GFUT1 deficiency syndrome | Trichorhinophalangeal syndrome type I |
| Greig cephalopolysyndactyly syndrome | Ulnar-mammary syndrome |
| Hereditary hemorrhagic telangiectasia type 1 | van der Woude syndrome-1 |
| Holoprosencephaly-3 | Waardenburg syndrome type 1 |
| Holoprosencephaly-4 | Waardenburg syndrome type 2A |
| Holoprosencephaly-5 | Waardenburg syndrome type 4C |

TABLE 20
Exemplary genes with ORFs comprising a PTC and exemplary disorders

| Gene | Disease/Disorder |
|---|---|
| AAAS | Glucocorticoid deficiency with achalasia |
| AAGAB | Keratosis palmoplantaris papulosa |
| AASS | Hyperlysinemia |
| ABCA1 | Tangier disease |
| ABCA12, | Autosomal recessive congenital ichthyosis-4B |
| SNHG31 | |
| ABCA3 | Surfactant metabolism dysfunction-3 |
| ABCA4 | Bietti crystalline corneoretinal dystrophy; Cone-rod degeneration; Cone-rod |
| | dystrophy-3; Macular dystrophy; Retinal dystrophy; Retinitis pigmentosa; |
| | Retinitis pigmentosa-19; Stargardt disease; Stargardt disease type 1 |
| ABCB4 | Intrahepatic cholestasis of pregnancy-3; Progressive familial intrahepatic |
| | cholestasis-3 |
| ABCC2 | Dubin-Johnson syndrome |
| ABCC6 | Cutis laxa; Generalized arterial calcification of infancy-2; Papule; |
| | Pseudoxanthoma elasticum forme fruste |
| ABCC8 | Familial hyperinsulinism; Familial hyperinsulinemic hypoglycemia-1 |
| ABCC9 | Arrhythmogenic right ventricular cardiomyopathy; Cardiomyopathy; |
| | Cardiovascular phenotype; Dilated cardiomyopathy-10; Primary dilated |
| | cardiomyopathy |
| ABCD1 | Adrenoleukodystrophy; Spastic gait; Spastic paraplegia |
| ABHD12 | Polyneuropathy; Cataract; Ataxia; Hearing loss; Retinitis pigmentosa |
| ABRAXAS1 | Hereditary breast and ovarian cancer syndrome |
| ACAD9 | Deficiency of acyl-CoA dehydrogenase family member 9 |
| ACADM | Medium-chain acyl-coenzyme A dehydrogenase deficiency |
| ACADS | Deficiency of butyryl-CoA dehydrogenase |
| ACADVL | Very long chain acyl-CoA dehydrogenase deficiency |
| ACAN | Osteochondritis dissecans; Kimberly type spondyloepiphyseal dysplasia |
| ACAT1 | Deficiency of acetyl-CoA acetyltransferase |
| ACBD5 | Retinal dystrophy with leukodystrophy |
| ACBD6, LHX4, | Short stature; Pituitary and cerebellar defects; Small sella turcica syndrome |
| LHX4-AS1 | |
| ACE | Renal dysplasia |
| ACOX1 | Peroxisomal acyl-CoA oxidase deficiency |
| ACP5 | Spondyloenchondrodysplasia with immune dysregulation |
| ACP5, ZNF627 | Spondyloenchondrodysplasia with immune dysregulation |
| ACTA1 | Congenital myopathy with excess of thin filaments |
| ACTB | Baraitser-Winter syndrome |
| ACVRL1 | Hereditary hemorrhagic telangiectasia type 1; Primary pulmonary |
| | hypertension; Pulmonary arterial hypertension related to hereditary |
| | hemorrhagic telangiectasia; ; Hereditary hemorrhagic; Telangiectasia type 2 |
| ACY1 | Neurological conditions associated with aminoacylase-1 deficiency |
| ADA | Severe combined immunodeficiency disease; Severe combined |
| | immunodeficiency due to ADA deficiency |
| ADAM10 | Reticulate acropigmentation of Kitamura |
| ADAMTS17 | Weill-Marchesani syndrome type 4 |
| ADAMTS2 | Dermatosparaxis-type Ehlers-Danlos syndrome |
| ADAMTSL4 | Ectopia lentis; Ectopia lentis et pupillae; Isolated autosomal recessive |
| | ectopia lentis-2 |
| ADCY3 | Body mass index quantitative trait locus-19 |
| ADCY3, | Body mass index quantitative trait locus-19 |
| CENPO | |
| ADGRG1 | Bilateral frontoparietal polymicrogyria |
| ADGRG2 | Congenital bilateral aplasia of vas deferens from CFTR mutation; X-linked |
| | congenital bilateral aplasia of vas deferens |
| ADGRG6 | Arthrogryposis multiplex congenita; Lethal congenital contracture |
| | syndrome-9 |
| ADGRV1 | Familial febrile seizures-4; Rare genetic deafness; Retinal dystrophy; Usher |
| | syndrome type 2C |
| ADNP | Helsmoortel-Van der Aa Syndrome; History of neurodevelopmental |
| | disorder; Inborn genetic diseases |
| AEBP1 | Classic-like Ehlers-Danlos syndrome-2 |
| AGA | Aspartylglucosaminuria |
| AGK | Sengers syndrome |
| AGK, | Congenital autosomal recessive cataract-5; Sengers syndrome |
| DENND11 | |
| AGL | Glycogen storage disease; Glycogen storage disease type IIIa; Glycogen |
| | storage disease type IIIb; Glycogen storage disease type III |
| AGPAT2 | Congenital generalized lipodystrophy type 1 |
| AGRN | Congenital myasthenic syndrome |
| AGT | Renal dysplasia |
| AGTR1 | Renal dysplasia |
| AGXT | Primary hyperoxaluria type I |
| AHDC1 | Delayed speech and language development; Global developmental delay; |
| | Intellectual disability; Muscular hypotonia; Neonatal hypotonia; Sleep |
| | apnea; Xia-Gibbs syndrome |
| AHI1 | Joubert syndrome; Joubert syndrome-3; Retinal dystrophy; Retinitis |
| | pigmentosa |
| AHR | Retinitis pigmentosa-85 |
| AIRE | Autoimmune polyglandular syndrome type 1; Autoimmune polyglandular |
| | syndrome type 1 with reversible metaphyseal dysplasia |
| ALB | Analbuminemia |
| ALDH18A1 | Cutis laxa; Corneal clouding; Oligophrenia syndrome |
| ALDH3A2 | Sjogren-Larsson syndrome |
| ALDH5A1 | Succinate-semialdehyde dehydrogenase deficiency |
| ALDH7A1 | Pyridoxine-dependent epilepsy; Seizures |
| ALDOB | Hereditary fructosuria |
| ALG1 | Congenital disorder of glycosylation |
| ALG3 | Congenital disorder of glycosylation |
| ALMS1 | Alstrom syndrome |
| ALOX12B | Autosomal recessive congenital ichthyosis-2 |
| ALPK3 | Familial hypertrophic cardiomyopathy-27; Hypertrophic cardiomyopathy |
| ALPL | Hypophosphatasia; Infantile hypophosphatasia |
| ALS2 | Amyotrophic lateral sclerosis type 2; Infantile-onset ascending hereditary |
| | spastic paralysis; Juvenile primary lateral sclerosis |
| ALX4 | Parietal foramina-2 |
| AMPD2 | Pontocerebellar hypoplasia type 9 |
| AMT | Non-ketotic hyperglycinemia |
| ANAPC1 | Rothmund-Thomson syndrome type 1 |
| ANGPTL3, | Familial h; ypobetalipoproteinemia-2 |
| DOCK7 | |
| ANKRD1 | ANKRD1-related dilated cardiomyopathy; Cardiovascular phenotype; |
| | Primary dilated cardiomyopathy |
| ANKRD11 | Abnormal facial shape; Clinodactyly of the 5th finger; Conductive hearing |
| | impairment; Delayed speech and language development; Global |
| | developmental delay; Inborn genetic diseases; Intellectual disability; KBG |
| | syndrome; Ptosis; Seizures; Short foot; Short palm; Unilateral |
| | cryptorchidism |
| ANO10 | Autosomal recessive cerebellar ataxia; Autosomal recessive spinocerebellar |
| | ataxia-10 |
| ANO5 | ANO5-related disorders; Achilles tendon contracture; Elevated serum |
| | creatine phosphokinase; Gnathodiaphyseal dysplasia; Limb-girdle muscular |
| | dystrophy; Lower limb amyotrophy; Lower limb muscle weakness; Miyoshi |
| | muscular dystrophy-3; Muscular diseases; Polycystic kidney dysplasia type |
| | 2L |
| ANTXR1 | Odontotrichomelic syndrome |
| AP1B1 | Autosomal recessive keratitis-ichthyosis-deafness syndrome |
| AP3B1 | Hermansky-Pudlak syndrome type 2 |
| AP4B1, AP4B1- | Inborn genetic diseases; Autosomal recessive spastic paraplegia-47 |
| AS1 | |
| AP4M1 | Autosomal recessive spastic paraplegia-50 |
| AP5Z1 | Autosomal recessive spastic paraplegia-48 |
| APC | Adenomatous colonic polyposis; Adenomatous polyposis coli with |
| | congenital cholesteatoma; Brain tumor-polyposis syndrome-2; Carcinoma |
| | of colon; Colon adenocarcinoma; Colorectal cancer; Craniopharyngioma; |
| | Desmoid disease; Desmoid tumors; Duodenal polyposis; Familial |
| | adenomatous polyposis; Familial adenomatous polyposis-1; Familial |
| | multiple polyposis syndrome; Gardner syndrome; Gastric polyposis; |
| | Hepatocellular carcinoma; Hereditary cancer-predisposing syndrome; |
| | Hyperplastic colonic polyposis; Intestinal polyp; Malignant colorectal |
| | neoplasm; Neoplasm of stomach; Neoplasm of the large intestine; |
| | Periampullary adenoma |
| APOA1, | Familial hypoalphalipoproteinemia |
| APOA1-AS | |
| APOB | Familial hypobetalipoproteinemia-1; Normotriglyceridemic familial |
| | hypobetalipoproteinemia |
| APOC2 | Nijmegen apolipoprotein C-II deficiency; Apolipoprotein C2 deficiency |
| APOC2, | Padova apolipoprotein C-II deficiency; Apolipoprotein C2 deficiency |
| APOC4-APOC2 | |
| APTX | Ataxia-oculomotor apraxia type 1 |
| AR | Androgen resistance syndrome; X-linked bulbo-spinal atrophy; Partial |
| | androgen insensitivity syndrome |
| ARCN1 | Rhizolemic short stature, micrognathia, and developmental delay with |
| | microcephaly |
| ARG1, MED23 | Arginase deficiency |
| ARHGEF18 | Retinitis pigmentosa-78 |
| ARID1A | Autosomal dominant mental retardation-14 |
| ARID1B | Absent speech; Blepharophimosis; Coffin-Siris syndrome-1; Constipation; |
| | Decreased body weight; Failure to thrive; Inborn genetic diseases; |
| | Intellectual disability; Long eyelashes; Microcephaly; Recurrent respiratory |
| | infections; Seizures; Short stature; Thick lower lip vermilion; Thin upper lip |
| | vermilion |
| ARID2 | Coffin-Siris syndrome type 6 |
| ARL2BP | Retinitis pigmentosa-82 with or without situs inversus |
| ARMC2 | Male infertility with teratozoospermia due to single gene mutation; |
| | Spermatogenic failure-38; Sperm tail anomaly |
| ARMC2, | Male infertility with teratozoospermia due to single gene mutation; |
| ARMC2-AS1 | Spermatogenic failure-38 |
| ARMC5 | Acth-independent macronodular adrenal hyperplasia-2 |
| ARSA | Late infantile metachromatic leukodystrophy; Pseudoarylsulfatase A |
| | deficiency |
| ARSB | Metachromatic leukodystrophy; Mucopolysaccharidosis type 6 |
| ART4 | Dombrock blood group system |
| ASAH1 | Farber disease; Spinal muscular atrophy-progressive myoclonic epilepsy |
| | syndrome |
| ASL | Argininosuccinate lyase deficiency |
| ASPA, | Canavan disease; Familial spongy degeneration of central nervous system |
| SPATA22 | |
| ASPM | Microcephaly; Primary autosomal recessive microcephaly; Primary |
| | autosomal recessive microcephaly-1; Primary autosomal recessive |
| | microcephaly-5 |
| ASS1 | Citrullinemia type I |
| ASXL1 | Bohring-Opitz syndrome; Inborn genetic diseases |
| ASXL3 | Bainbridge-Ropers syndrome |
| ATF6 | Achromatopsia; Achromatopsia-7 |
| ATL1 | Hereditary spastic paraplegia-3A |
| ATM | Ataxia-telangiectasia syndrome; Familial cancer of breast; Hereditary breast |
| | and ovarian cancer syndrome; Hereditary cancer-predisposing syndrome; |
| | Ovarian neoplasms |
| ATM, C11orf65, | Ataxia-telangiectasia syndrome; Ataxia-telangiectasia without |
| ATP13A2 | immunodeficiency; Breast cancer; Familial cancer of breast; Hereditary |
| | breast and ovarian cancer syndrome; Hereditary cancer-predisposing |
| | syndrome; Neoplasm of the breast; Susceptibility to Kufor-Rakeb syndrome |
| ATP1A2 | Abnormality of neuronal migration; Arthrogryposis multiplex congenita; |
| | Epilepsy; Hydrops fetalis |
| ATP2A1 | Brody myopathy |
| ATP2C1 | Familial benign pemphigus |
| ATP6V0A2 | ALG9 congenital disorder of glycosylation; Cutis laxa with osteodystrophy |
| ATP6V0A4 | Autosomal recessive distal renal tubular acidosis |
| ATP7A | Cutis laxa; X-linked Menkes kinky-hair syndrome |
| ATP7B | Inborn genetic diseases; Wilson disease |
| ATRX | Alpha thalassemia-X-linked intellectual disability syndrome; Intellectual |
| | disability; Mental retardation-hypotonic facies syndrome; X-linked mental |
| | retardation-hypotonic facies syndrome-1 |
| AXIN2 | Oligodontia-colorectal cancer syndrome |
| B3GALNT1 | P-blood group phenotype |
| B3GALNT2 | Congenital muscular dystrophy-dystroglycanopathy type A-11 with brain |
| | and eye anomalies |
| B3GALT6 | Spondylo-epi-metaphyseal dysplasia |
| B4GALNT1 | Hereditary spastic paraplegia-26; Inborn genetic diseases |
| B4GALT7 | Progeroid type Ehlers-Danlos syndrome |
| B9D1 | Joubert syndrome; Meckel syndrome; Meckel-Gruber syndrome type 9 |
| B9D2 | Joubert syndrome |
| BAG3 | BAG3-related cardiovascular phenotype; Dilated cardiomyopathy-1HH; |
| | Inborn genetic diseases; Myofibrillar myopathy; Primary dilated |
| | cardiomyopathy |
| BAP1 | Hereditary cancer-predisposing syndrome; Tumor susceptibility linked to |
| | germline BAP1 mutations |
| BARD1 | Breast cancer; Familial cancer of breast; Hereditary breast and ovarian |
| | cancer syndrome; Hereditary cancer-predisposing syndrome; Triple- |
| | negative breast cancer |
| BBS1 | Bardet-Biedl syndrome |
| BBS1, | Bardet-Biedl syndrome; Bardet-Biedl syndrome-1 |
| ZDHHC24 | |
| BBS10 | Bardet-Biedl syndrome; Bardet-Biedl syndrome-1; Bardet-Biedl syndrome- |
| | 10; Digenic Bardet-biedl syndrome-6/10; Inborn genetic diseases; Retinal |
| | dystrophy; Retinitis pigmentosa |
| BBS2 | Bardet-Biedl syndrome; Bardet-Biedl syndrome-2; Digenic Bardet-Biedl |
| | syndrome-1/2; Digenic Bardet-biedl syndrome-2/6; Retinal dystrophy; |
| | Retinitis pigmentosa; Retinitis pigmentosa-74 |
| BBS5 | Bardet-Biedl syndrome-5 |
| BBS9 | Bardet-Biedl syndrome |
| BCKDHA | Maple syrup urine disease; Maple syrup urine disease type-1A |
| BCKDHB | Classic; maple syrup urine disease type 1B; Maple syrup urine disease; |
| | Maple syrup urine disease type 1B |
| BCOR | Oculofaciocardiodental syndrome |
| BCS1L | BCS1L-related disorders; Gracile syndrome; Leigh syndrome; |
| | Mitochondrial complex III deficiency nuclear type 1; Pili torti-deafness |
| | syndrome |
| BEST1 | Autosomal recessive bestrophinopathy; Retinal dystrophy; Vitelliform |
| | macular dystrophy type 2 |
| BET1 | Progressive muscle weakness; Seizures |
| BFSP1 | Cataract-33 (multiple types) |
| BLM | Bloom syndrome; Hereditary breast and ovarian cancer syndrome; |
| | Hereditary cancer-predisposing syndrome |
| BMP1 | Osteogenesis imperfecta type XIII |
| BMP2 | Skeletal anomalies with or without cardiac anomalies; Facial dysmorphism; |
| | Short stature |
| BMPR1A | Hereditary cancer-predisposing syndrome; Juvenile polyposis syndrome |
| BMPR2 | Primary pulmonary hypertension |
| BNC1 | Premature ovarian failure-16 |
| BOLA3 | Multiple mitochondrial dysfunctions syndrome-2 |
| BPNT2 | GPAPP type chondrodysplasia with joint dislocations |
| BPTF | Neurodevelopmental disorder with dysmorphic facies and distal limb |
| | anomalies |
| BRAT1 | Inborn genetic diseases; Neurodevelopmental disorder with cerebellar |
| | atrophy and with or without seizures; Lethal neonatal rigidity and |
| | multifocal seizure syndrome |
| BRCA1 | Breast and/or ovarian cancer; Breast carcinoma; Familial breast-ovarian |
| | cancer-1; Dysgerminoma; Complementation group S Fanconi anemia; |
| | Familial cancer of breast; Hereditary breast and ovarian cancer syndrome; |
| | Hereditary cancer-predisposing syndrome; Infiltrating duct carcinoma of |
| | breast; Neoplasm of ovary; Neoplasm of the breast; Ovarian neoplasms; |
| | Ovarian serous surface papillary adenocarcinoma; Ovarian cancer; |
| | Pancreatic cancer; Pancreatic cancer-4; Porokeratosis punctata palmaris et |
| | plantaris; Rhabdomyosarcoma; Bilateral breast cancer; Breast cancer |
| BRCA2 | Asthma; BRCA2-related disorders; Breast and/or ovarian cancer; Breast |
| | carcinoma; Breast-ovarian cancer; Cancer of the pancreas; Colorectal |
| | cancer; Diffuse intrinsic pontine glioma; Ectopic ossification; Familial |
| | cancer of breast; Complementation group D1 Fanconi anemia; Focal |
| | seizures; Genetic non-acquired premature ovarian failure; Glioma |
| | susceptibility-3; Headache; Hereditary cancer syndrome; Hereditary breast |
| | and ovarian cancer syndrome; Hereditary cancer-predisposing syndrome; |
| | Inborn genetic diseases; Malignant tumor of prostate; Medulloblastoma; |
| | Migraine; Muscle weakness; Neoplasm of the breast; Nephrolithiasis; |
| | Obesity; Ovarian neoplasms; Ovarian cancer; Pancreatic cancer-2; |
| | Polydactyly; Short attention span; Striae distensae; Tracheoesophageal |
| | fistula; Tumor susceptibility linked to germline BAP1 mutations; Wilms |
| | tumor-1 |
| BRIP1 | BRIP1-related disorders; Breast cancer; Carcinoma of colon; Familial |
| | cancer of breast; Complementation group J Fanconi anemia; Hereditary |
| | breast and ovarian cancer syndrome; Hereditary cancer-predisposing |
| | syndrome; Neoplasm of ovary; Neoplasm of the breast; Ovarian Cancers; |
| | Ovarian Neoplasms; Tracheoesophageal fistula |
| BRWD3 | X-linked mental retardation-93 |
| BSND | Bartter disease type 4A |
| BTD | Biotinidase deficiency |
| BTK | Non-Bruton agammaglobulinemia; X-linked agammaglobulinemia; X- |
| | linked agammaglobulinemia with growth hormone deficiency |
| C11orf65, ATM | Ataxia-telangiectasia syndrome; Hereditary breast and ovarian cancer |
| | syndrome; Hereditary cancer-predisposing syndrome |
| C12orf4 | Autosomal recessive mental retardation-66; Attention deficit hyperactivity |
| | disorder; Intellectual disability; Muscular hypotonia |
| C12orf65 | Combined oxidative phosphorylation deficiency-7; Spastic paraplegia |
| C19orf12 | Neurodegeneration with brain iron accumulation-4; Autosomal recessive |
| | spastic paraplegia-43 |
| C1QB | C1q deficiency |
| C1S | Complement component C1s deficiency |
| C2 | Complement component 2 deficiency |
| C2CD3 | Orofaciodigital syndrome-XIV |
| C5 | Leiner disease |
| C6 | Complement component 6 deficiency; Immunodeficiency due to a late |
| | component of complement deficiency |
| C7 | Complement component 7 deficiency |
| C8B | Complement component 6 deficiency; Type II complement component 8 |
| | deficiency |
| C8orf37 | Cone-rod dystrophy-16 |
| C8orf37 | Retinitis pigmentosa-64 |
| CA2 | Osteopetrosis with renal tubular acidosis |
| CABP4 | Congenital stationary night blindness type 2B |
| CACNA1A | Bulbar palsy; early infantile epileptic encephalopathy-42; Episodic ataxia; |
| | Episodic ataxia type 2; Recurrent respiratory infections and epilepsy |
| CACNA1C | Long QT syndrome |
| CACNA2D4 | Abnormality of the eye; Retinal cone dystrophy-4 |
| CAPN1 | Autosomal recessive spastic paraplegia-76; |
| CAPN3 | Absent Achilles reflex; Absent muscle fiber calpain-3; Arrhythmia; Calf |
| | muscle hypertrophy; Congenital muscular dystrophy; Contractures of the |
| | joints of the lower limbs; Difficulty walking; EMG: myopathic |
| | abnormalities; EMG: neuropathic changes; Elbow flexion contracture; |
| | Elevated serum creatine phosphokinase; Recessive limb-girdle muscular |
| | dystrophy type 2A; Limb-girdle muscle weakness; Limb-girdle muscular |
| | dystrophy; Migraine; Muscle weakness; Muscular diseases; Muscular |
| | dystrophy; Eosinophilic myositis; Paresthesia; Positive Romberg sign; |
| | Progressive spinal muscular atrophy; Shoulder girdle muscle weakness |
| CASK | Mental retardation and microcephaly with pontine and cerebellar hypoplasia |
| CASP14 | Congenital autosomal recessive ichthyosis-12 |
| CASQ2 | Catecholaminergic polymorphic ventricular tachycardia-2 |
| CASR | Familial hypocalciuric hypercalcemia type 1; Inborn genetic diseases |
| CAST | Peeling skin with leukonychia, acral punctate keratoses, and knuckle pads; |
| | Cheilitis |
| CAST, ERAP1 | Peeling skin with leukonychia, acral punctate keratoses, and knuckle pads; |
| | Cheilitis |
| CAT | Acatalasemia; Japanese type acatalasia |
| CATSPER1 | Spermatogenic failure-7 |
| CAV1 | Congenital generalized lipodystrophy type 3 |
| CAV3, SSUH2 | Long QT syndrome |
| CBL | Noonan syndrome-like disorder with or without juvenile myelomonocytic |
| | leukemia |
| CBS | Cystathionine beta-synthetase polymorphism; Classic homocystinuria; |
| | Homocystinuria |
| CC2D1A | Psychosocial mental retardation; Autosomal recessive mental retardation-3 |
| CC2D2A | Joubert syndrome; Joubert syndrome-9; Meckel syndrome type 6; Meckel- |
| | Gruber syndrome |
| CCBE1 | Hennekam lymphangiectasia-lymphedema syndrome-1 |
| CCDC103 | Primary ciliary dyskinesia |
| CCDC28B | Bardet-Biedl syndrome; Bardet-Biedl syndrome-1 |
| CCDC39 | Primary ciliary dyskinesia-14; Primary ciliary dyskinesia |
| CCDC40 | Primary ciliary dyskinesia-15; Primary ciliary dyskinesia |
| CCDC47 | Global developmental delay with dysmorphic features, woolly hair, liver |
| | dysfunction, and pruritus; Trichohepatoneurodevelopmental syndrome |
| CCDC65 | Primary ciliary dyskinesia-27; Kartagener syndrome; Primary ciliary |
| | dyskinesia |
| CCDC78 | Centronuclear myopathy-4 |
| CCDC88C | Congenital hydrocephalus-1 |
| CCN6 | Progressive pseudorheumatoid dysplasia |
| CCNH, RASA1 | Capillary malformation; Arteriovenous malformation |
| CCNO | Primary ciliary dyskinesia-29; Kartagener syndrome; Primary ciliary |
| | dyskinesia |
| CCNQ | Syndactyly-telecanthus-anogenital and renal malformations syndrome |
| CD19 | Common variable immunodeficiency-3 |
| CD247 | Immunodeficiency due to defect in CD3-zeta |
| CD36 | Susceptibility to malaria; Platelet glycoprotein IV deficiency |
| CD46 | Atypical hemolytic-uremic syndrome-2 |
| CD55 | Cromer blood group system Dr(a-) phenotype; Protein-losing enteropathy |
| CDC14A | Autosomal recessive deafness-32; Rare genetic deafness |
| CDC73 | Parathyroid adenoma; Parathyroid carcinoma |
| CDH1 | Blepharocheilodontic syndrome-1; Lobular breast cancer; Endometrial |
| | carcinoma; Familial cancer of breast; Hereditary cancer-predisposing |
| | syndrome; Hereditary diffuse gastric cancer; Malignant tumor of prostate; |
| | Neoplasm of ovary |
| CDH11 | Brachioskeletogenital syndrome |
| CDH23 | Autosomal recessive deafness-12; Inborn genetic diseases; Pituitary |
| | adenoma-5 (multiple types); Rare genetic deafness; Usher syndrome type |
| | 1D |
| CDH23, | Rare genetic deafness |
| C10orf105 | |
| CDH23, | Digenic Usher syndrome type 1D/F; Usher syndrome type 1; Usher |
| CDH23-AS1 | syndrome type 1D |
| CDH3 | Congenital hypotrichosis with juvenile macular dystrophy; EEM syndrome; |
| | Hypotrichosis with juvenile macular dystrophy; Macular dystrophy |
| CDHR1 | Cone-rod dystrophy-15; Leber congenital amaurosis; Retinal dystrophy; |
| | Retinitis pigmentosa-65 |
| CDK10 | Al Kaissi syndrome |
| CDK13 | Congenital heart defects, intellectual developmental disorder, and |
| | dysmorphic facial features |
| CDK5RAP2 | Primary autosomal recessive microcephaly-3 |
| CDKL5 | Angelman syndrome-like; Atypical Rett syndrome; Early infantile epileptic |
| | encephalopathy-2; Epileptic encephalopathy; Inborn genetic diseases |
| CDKN2A | Hereditary cancer-predisposing syndrome; Hereditary cutaneous melanoma; |
| | Melanoma-pancreatic cancer syndrome; Neoplasm |
| CDSN, | Peeling skin syndrome-1 |
| PSORS1C1 | |
| CEL | Maturity-onset diabetes of the young type 8 |
| CELA2A | Coronary artery disease; Diabetes; Familial partial lipodystrophy-6; |
| | Hypertensive disorder; Hypertriglyceridemia |
| CENPF | Stromme syndrome |
| CENPJ | Congenital microcephaly; Intellectual disability; Perisylvian |
| | polymicrogyria; Primary autosomal recessive microcephaly; Primary |
| | autosomal recessive microcephaly-1; Primary autosomal recessive |
| | microcephaly-6; Seckel syndrome-4; Type III lissencephaly |
| CEP120 | Joubert syndrome-31 |
| CEP152 | Seckel syndrome |
| CEP290 | Abnormality of the kidney; Bardet-Biedl syndrome-14; Blindness; CEP290- |
| | related disorders; Cerebellar cyst; Cerebellar vermis hypoplasia; Global |
| | developmental delay; Hyperechogenic kidneys; Joubert syndrome; Joubert |
| | syndrome-5; Leber congenital amaurosis-10; Meckel syndrome type 4; |
| | Meckel-Gruber syndrome; Nephronophthisis; Polycystic kidney dysplasia; |
| | Retinal dystrophy; Senior-Loken syndrome-6 |
| CEP290, | Bardet-Biedl syndrome-14; Joubert syndrome; Joubert syndrome-5; |
| C12orf29 | Meckel-Gruber syndrome; Nephronophthisis |
| CEP41 | Joubert syndrome-15 |
| CEP78 | Cone-rod degeneration; Cone-rod dystrophy and hearing loss-1; |
| | Sensorineural hearing loss |
| CFAP251 | Male infertility with teratozoospermia due to single gene mutation; Non- |
| | syndromic male infertility due to sperm motility disorder; Spermatogenic |
| | failure-18; Spermatogenic failure-33; Asthenozoospermia; Dysplasia of the |
| | mitochondrial sheath; Multiple morphologic abnormalities of the sperm |
| | flagellum |
| CFAP410 | Axial spondylometaphyseal dysplasia; Retinal dystrophy with or without |
| | macular staphyloma |
| CFAP43 | Spermatogenic failure-19 |
| CFAP44 | Spermatogenic failure-20 |
| CFHR5 | CFHR5 deficiency |
| CFTR | Bronchiectasis with or without elevated sweat chloride-1; CFTR-related |
| | disorders; Congenital bilateral aplasia of vas deferens from CFTR mutation; |
| | Cystic fibrosis; Hereditary pancreatitis; Inborn genetic diseases; Ataluren |
| | response |
| CFTR, CFTR- | CFTR-related disorders; Congenital bilateral aplasia of vas deferens from |
| AS1 | CFTR mutation; Cystic fibrosis |
| CFTR, | Bronchiectasis with or without elevated sweat chloride-1; CFTR-related |
| LOC111674472 | disorders; Congenital bilateral aplasia of vas deferens from CFTR mutation; |
| | Cystic fibrosis; Hereditary pancreatitis |
| CFTR, | Bronchiectasis with or without elevated sweat chloride-1; CFTR-related |
| LOC111674475 | disorders; Congenital bilateral aplasia of vas deferens from CFTR mutation; |
| | Cystic fibrosis; Hereditary pancreatitis; Inborn genetic diseases; ataluren |
| | response |
| CFTR, | Cystic fibrosis |
| LOC111674477 | |
| CFTR, | Bronchiectasis with or without elevated sweat chloride-1; CFTR-related |
| LOC113633877 | disorders; Congenital bilateral aplasia of vas deferens from CFTR mutation; |
| | Cystic fibrosis; Hereditary pancreatitis |
| CFTR, | Bronchiectasis with or without elevated sweat chloride-1; Congenital |
| LOC113664106 | bilateral aplasia of vas deferens from CFTR mutation; Cystic fibrosis; |
| | Hereditary pancreatitis |
| CHD2 | CHD2-related disorder; Childhood-onset epileptic encephalopathy; History |
| | of neurodevelopmental disorder |
| CHD7 | CHARGE association; Hypogonadism with anosmia; Hypogonadotropic |
| | hypogonadism-5 with or without anosmia |
| CHEK2 | Astrocytoma; B Lymphoblastic leukemia/lymphoma; Breast and colorectal |
| | cancer; Breast cancer; CHEK2-related cancer susceptibility; Colitis; |
| | Congenital heart defects; Diffuse intrinsic pontine glioma; Familial cancer |
| | of breast; Hematochezia; Hereditary breast and ovarian cancer syndrome; |
| | Hereditary cancer; Hereditary cancer-predisposing syndrome; Inflammation |
| | of the large intestine; Leiomyosarcoma; Li-Fraumeni syndrome; Li- |
| | Fraumeni syndrome-2; Malignant tumor of prostate; Neoplasm of the |
| | breast; Osteosarcoma; Ovarian neoplasms; Prostate cancer; |
| | Thrombocytopenia |
| CHM | Retinal dystrophy |
| CHRDL1 | Megalocornea |
| CHRNA1 | Congenital myasthenic syndrome |
| CHRNA2 | Autosomal dominant nocturnal frontal lobe epilepsy |
| CHRNA3 | CHRNA3-related condition |
| CHRND | Lethal multiple pterygium syndrome |
| CHRNE | Slow-channel congenital myasthenic syndrome-4A; Congenital myasthenic |
| | syndrome-4C; Congenital slow-channel myasthenic syndrome |
| CHRNE, | Slow-channel congenital myasthenic syndrome-4A; Congenital myasthenic |
| C17orf107 | syndrome-4C; Fast-channel congenital myasthenic syndrome-4B |
| CHRNG | Autosomal recessive multiple pterygium syndrome; CHRNG-related |
| | disorders; Inborn genetic diseases; Lethal multiple pterygium syndrome |
| CHST14 | Musculocontractural type Ehlers-Danlos syndrome |
| CHST3 | Spondyloepiphyseal dysplasia with congenital joint dislocations |
| CHSY1 | Temtamy preaxial brachydactyly syndrome |
| CIB1 | Susceptibility to epidermodysplasia verruciformis-3 |
| CIITA | Bare lymphocyte syndrome-2 |
| CKAP2L | Filippi syndrome |
| CLCN1 | Autosomal dominant intermediate Charcot-Marie-Tooth disease; Congenital |
| | myotonia; EMG: myopathic abnormalities; Muscular diseases; Autosomal |
| | dominant myotonia congenita; Autosomal recessive myotonia congenita |
| CLCN2 | Leukoencephalopathy with ataxia; Juvenile myoclonic epilepsy-8 |
| CLCN5 | X-linked recessive nephrolithiasis; X-linked recessive nephrolithiasis with |
| | renal failure |
| CLDN1, | Neonatal ichthyosis-sclerosing cholangitis syndrome |
| CLDN16 | |
| CLIC5 | Autosomal recessive deafness |
| CLN3 | Juvenile neuronal ceroid lipofuscinosis; Neuronal ceroid lipofuscinosis |
| CLN5, FBXL3 | Neuronal ceroid lipofuscinosis; Neuronal ceroid lipofuscinosis-5 |
| CLRN1 | Rare genetic deafness; Retinal dystrophy; Retinitis pigmentosa; Usher |
| | syndrome type 3A |
| CNGA1, | Retinal dystrophy; Retinitis pigmentosa-49 |
| LOC101927157 | |
| CNGB1 | Retinal dystrophy; Retinitis pigmentosa; Retinitis pigmentosa-45 |
| CNGB3 | Abnormality of the eye; Achromatopsia; Achromatopsia-3; CNGB3-related |
| | disorders; Cone-rod dystrophy; Leber congenital amaurosis; Retinal |
| | dystrophy; Retinitis pigmentosa; Stargardt Disease |
| CNNM2 | Renal hypomagnesemia-6 |
| CNNM4 | Jalili syndrome |
| CNTNAP1 | Lethal congenital contracture syndrome-7 |
| CNTNAP2 | Pitt-Hopkins-like syndrome-1 |
| COASY | Neurodegeneration with brain iron accumulation-6 |
| COG4 | Congenital disorder of glycosylation type 2J |
| COG5 | Congenital disorder of glycosylation type 2I |
| COG5, DUS4L, | Congenital disorder of glycosylation type 2I |
| DUS4L- | |
| BCAP29 | |
| COL10A1 | Schmid type metaphyseal chondrodysplasia |
| COL11A1 | Fibrochondrogenesis-1 |
| COL12A1 | Ullrich congenital muscular dystrophy-2 |
| COL17A1 | Junctional epidermolysis bullosa non-Herlitz type; Junctional epidermolysis |
| | bullosa localisata variant |
| COL18A1 | Primary closed-angle glaucoma; Knobloch syndrome-1 |
| COL18A1, | Knobloch syndrome-1; Macular dystrophy; Retinal dystrophy; Retinitis |
| SLC19A1 | pigmentosa |
| COL1A1 | Procollagen proteinase deficient Ehlers-Danlos syndrome; Infantile cortical |
| | hyperostosis; Recessive perinatal lethal osteogenesis imperfecta; |
| | Osteogenesis imperfecta type I; Osteogenesis imperfecta type III; Dominant |
| | form osteogenesis imperfecta with normal sclerae; Postmenopausal |
| | osteoporosis |
| COL1A2 | COL1A2-related disorder; Autosomal recessive cardiac valvular form |
| | Ehlers-Danlos syndrome; Inborn genetic diseases; Osteogenesis imperfecta |
| | type I (classic type) |
| COL2A1 | Spondyloperipheral dysplasia-short ulna syndrome; Stickler syndrome type |
| | 1 |
| COL3A1 | Ehlers-Danlos syndrome; type 4 |
| COL4A3, MFF- | Autosomal recessive Alport syndrome |
| DT | |
| COL4A5 | X-linked recessive Alport syndrome-1 |
| COL5A1 | Classic type Ehlers-Danlos syndrome |
| COL5A2 | Classic type Ehlers-Danlos syndrome; Ehlers-Danlos syndrome classic |
| | type-2 |
| COL6A1 | Bethlem myopathy-1 |
| COL6A2 | Bethlem myopathy-1; Ullrich congenital muscular dystrophy-1 |
| COL6A3 | Bethlem myopathy-1 |
| COL7A1 | Autosomal dominant dystrophic epidermolysis bullosa; Epidermolysis |
| | bullosa pruriginosa; Recessive dystrophic epidermolysis bullosa; Transient |
| | bullous dermolysis of the newborn |
| COL9A2 | Stickler syndrome type 5 |
| COLEC10 | 3MC syndrome-3 |
| COLEC10, | 3MC syndrome-3 |
| LOC101927513 | |
| COLQ | Congenital myasthenic syndrome; Endplate acetylcholinesterase deficiency |
| COQ2 | Primary coenzyme Q10 deficiency-1 |
| COQ8A | ADCK3-related disorders; Primary coenzyme Q10 deficiency-4 |
| COQ9 | Primary coenzyme Q10 deficiency-5 |
| COX15 | Fatal infantile cardioencephalomyopathy due to cytochrome c oxidase |
| | deficiency-2; Leigh syndrome; Leigh syndrome due to mitochondrial |
| | complex IV deficiency |
| CP | Ceruloplasmin belfast; Deficiency of ferroxidase; Systemic hemosiderosis |
| | due to aceruloplasminemia |
| CPAMD8 | Anterior segment dysgenesis-8 |
| CPLANE1 | Global developmental delay; Jaundice; Joubert syndrome; Joubert |
| | syndrome-1; Joubert syndrome-17; Orofaciodigital syndrome type 6; |
| | Typical Joubert syndrome MRI findings |
| CPOX | Coproporphyria |
| CPS1 | Congenital hyperammonemia type I |
| CPSF1 | Myopia-27 |
| CPT2 | Infantile carnitine palmitoyltransferase II deficiency; Lethal neonatal |
| | carnitine palmitoyltransferase II deficiency; Myopathic stress-induced |
| | carnitine palmitoyltransferase II deficiency |
| CRB1 | Leber congenital amaurosis-8 |
| CRB2 | Focal segmental glomerulosclerosis-9; Steroid-resistant nephrotic syndrome |
| CRIPT | Ateleiotic dwarfism; Short stature with microcephaly and distinctive facies |
| CRPPA | Congenital muscular dystrophy-dystroglycanopathy with brain and eye |
| | anomalies type A7; Limb-girdle muscular dystrophy-dystroglycanopathy |
| | type C7 |
| CRTAP | Osteogenesis imperfecta type 7 |
| CRX | Leber congenital amaurosis-7 |
| CRYAB | Alpha-B crystallinopathy; Dilated cardiomyopathy-1II |
| CRYBA4, | Autosomal recessive congenital nuclear cataract-3 |
| CRYBB1 | |
| CRYBB2 | Cataract-3; Congenital cataract |
| CSGALNACT1 | Mild skeletal dysplasia with joint laxity and advanced bone age |
| CSPP1 | Joubert syndrome-21; Meckel-Gruber syndrome |
| CSRP3 | Cardiovascular phenotype |
| CSTB | Inborn genetic diseases; Progressive myoclonic epilepsy; Unverricht- |
| | Lundborg syndrome |
| CTC1 | Cerebroretinal microangiopathy with calcifications and cysts; |
| | Cerebroretinal microangiopathy with calcifications and cysts-1; |
| | Dyskeratosis congenita |
| CTCF | Autosomal dominant mental retardation-21 |
| CTNNB1 | Exudative vitreoretinopathy-7; Exudative vitreoretinopathy-1; |
| | Hepatocellular carcinoma; Inborn genetic diseases; Autosomal dominant |
| | mental retardation-19 |
| CTNND1, | Blepharocheilodontic syndrome-2 |
| TMX2- | |
| CTNND1 | |
| CTNS | Cystinosis; Juvenile nephropathic cystinosis; Nephropathic cystinosis; |
| | Ocular cystinosis |
| CTSD | Neuronal ceroid lipofuscinosis-10 |
| CTSH | Variant of unknown significance |
| CTU2 | Microcephaly, facial dysmorphism, renal agenesis, and ambiguous genitalia |
| | syndrome |
| CUBN | Megaloblastic anemia due to inborn errors of metabolism |
| CUL4B | Cabezas type syndromic X-linked mental retardation |
| CUL7 | Three M syndrome-1 |
| CWC27 | Retinitis pigmentosa with or without skeletal anomalies |
| CWF19L1 | Autosomal recessive spinocerebellar ataxia-17 |
| CYB5R3 | Methemoglobinemia type 2 |
| CYBB | X-linked chronic granulomatous disease |
| CYP11B1, | Deficiency of steroid 11-beta-monooxygenase |
| LOC106799833 | |
| CYP17A1 | Combined partial 17-alpha-hydroxylase/17/20-lyase deficiency; Complete |
| | combined 17-alpha-hydroxylase/17/20-lyase deficiency; Deficiency of |
| | steroid 17-alpha-monooxygenase |
| CYP1B1 | Anterior segment dysgenesis-6; CYP1B1-related disorders; Congenital |
| | glaucoma; Congenital ocular coloboma; Primary congenital glaucoma-3A; |
| | Primary infantile glaucoma-3B; Irido-corneo-trabecular dysgenesis |
| CYP21A2, | Classic congenital adrenal hyperplasia due to 21-hydroxylase deficiency |
| LOC106780800 | |
| CYP21A2, | Classic congenital adrenal hyperplasia due to 21-hydroxylase deficiency |
| TNXB, | |
| LOC106780800 | |
| CYP24A1 | Infantile hypercalcemia-1 |
| CYP26C1 | Optic nerve hypoplasia |
| CYP27A1 | Cholestanol storage disease |
| CYP27B1 | Vitamin D-dependent rickets type 1 |
| CYP2C19 | CYP2C19-related poor metabolism of clopidogrel, mephenytoin, proguanil, |
| | amitriptyline, citalopram, clomipramine, and clopidogrel |
| CYP2D6 | CYP2D6-related poor metabolism of debrisoquine, deutetrabenazine, |
| | tamoxifen, tramadol, amitriptyline, antidepressants, clomipramine, |
| | desipramine, doxepin, imipramine, nortriptyline, tamoxifen, and |
| | trimipramine |
| CYP2U1 | Autosomal recessive spastic paraplegia-56 |
| CYP4F22 | Autosomal recessive congenital ichthyosis-5 |
| CZ1P-ASNS, | Asparagine synthetase deficiency |
| ASNS | |
| DBH | Orthostatic hypotension-1 |
| DBT | Maple syrup urine disease; Maple syrup urine disease type 2 |
| DCAF17 | Hypogonadism, alopecia, diabetes mellitus, mental retardation, and |
| | electrocardiogramormalities |
| DCLRE1C | Partial severe combined immunodeficiency; Severe combined |
| | immunodeficiency due to DCLRE1C deficiency |
| DCN | Congenital stromal corneal dystrophy |
| DDHD1 | Autosomal recessive spastic paraplegia-28 |
| DDRGK1 | Shohat type spondyloepimetaphyseal dysplasia |
| DDX3X | Delayed speech and language development; Global developmental delay; |
| | History of neurodevelopmental disorder; X-linked mental retardation-102; |
| | Microcephaly |
| DDX41 | Acute myeloid leukemia; Susceptibility to familial myeloproliferative, |
| | lymphoproliferative neoplasms |
| DEPDC5 | DEPDC5-related disorder; Familial focal epilepsy with variable foci |
| DES | Limb-girdle muscular dystrophy type 2R; Myofibrillar myopathy-1; |
| | Neuromuscular disease; Primary dilated cardiomyopathy |
| DGKE | Nephrotic syndrome type 7 |
| DGUOK | Hepatocerebral form mitochondrial DNA depletion syndrome due to |
| | DGUOK deficiency; Hepatocerebral mitochondrial DNA depletion |
| | syndrome-3; Autosomal recessive progressive external ophthalmoplegia |
| | with mitochondrial DNA deletions-4 |
| DHCR7 | 2-3 toe syndactyly; Congenital microcephaly; Elevated 7- |
| | dehydrocholesterol; History of neurodevelopmental disorder; Inborn genetic |
| | diseases; Small for gestational age; Smith-Lemli-Opitz syndrome |
| DHH | 46, XY sex reversal type 7 |
| DHTKD1 | 2-aminoadipic 2-oxoadipic aciduria |
| DIAPH1 | Seizures, cortical blindness, and microcephaly syndrome |
| DICER1 | DICER1-related pleuropulmonary blastoma cancer predisposition |
| | syndrome; Hereditary cancer-predisposing syndrome |
| DIPK1A, RPL5 | Diamond-Blackfan anemia-6 |
| DLD | Maple syrup urine disease type 3 |
| DLG3 | X-Linked mental retardation-90 |
| DLL3, | Leukodystrophy and acquired microcephaly with or without dystonia; |
| PLEKHG2 | Autosomal recessive spondylocostal dysostosis-1 |
| DLX3 | Amelogenesis imperfecta type IV; Tricho-dento-osseous syndrome |
| DLX4 | Orofacial cleft-15 |
| DMD | Becker muscular dystrophy; Duchenne muscular dystrophy |
| DMP1 | Autosomal recessive hypophosphatemic vitamin D refractory rickets |
| DNAAF2 | Primary ciliary dyskinesia |
| DNAAF4, | Primary ciliary dyskinesia |
| DNAAF4- | |
| CCPG1 | |
| DNAH1 | Non-syndromic male infertility due to sperm motility disorder; |
| | Spermatogenic failure-18 |
| DNAH11 | Primary ciliary dyskinesia-7; Primary ciliary dyskinesia |
| DNAH17 | Spermatogenic failure-39 |
| DNAH5 | Primary ciliary dyskinesia-3; Primary ciliary dyskinesia |
| DNAI1 | Kartagener syndrome; Primary ciliary dyskinesia |
| DNAI2 | Primary ciliary dyskinesia-9; Primary ciliary dyskinesia |
| DNAJB2 | Charcot-Marie-Tooth disease; Autosomal recessive distal spinal muscular |
| | atrophy-5 |
| DNAJC12 | Mild non-BH4-deficient hyperphenylalaninemia |
| DNAL1 | Primary ciliary dyskinesia-16; Primary ciliary dyskinesia |
| DNM2 | Intermediate dominant Charcot-Marie-Tooth disease-B |
| DNMBP | Cataract-48 |
| DOCK6 | Adams-Oliver syndrome-2 |
| DOCK6, | Adams-Oliver syndrome; Adams-Oliver syndrome-2 |
| LOC105372273 | |
| DOCK8 | Autosomal recessive hyperimmunoglobulin E recurrent infection syndrome; |
| | Inborn genetic diseases |
| DOK7 | Congenital myasthenic syndrome; Inborn genetic diseases; Familial limb- |
| | girdle myasthenia; Pena-Shokeir syndrome type I |
| DOLK | Congenital disorder of glycosylation type 1M |
| DONSON | Microcephaly short stature and limb abnormalities; Microcephaly- |
| | micromelia syndrome |
| DPY19L2 | Spermatogenic failure-9 |
| DPYD | Dihydropyrimidine dehydrogenase deficiency; Fluorouracil response |
| DRAM2 | Cone-rod dystrophy 21; Retinal dystrophy |
| DRC1 | Primary ciliary dyskinesia-21; Kartagener syndrome; Primary ciliary |
| | dyskinesia |
| DSC2 | Arrhythmogenic right ventricular cardiomyopathy with mild palmoplantar |
| | keratoderma and woolly hair; Familial arrhythmogenic right ventricular |
| | dysplasia type 11 |
| DSC2, DSCAS | Arrhythmogenic right ventricular cardiomyopathy type 11 |
| DSG1 | Focal, striate, or diffuse palmoplantar keratoderma-I |
| DSG1, DSG1- | Congenital erythroderma with palmoplantar keratoderma, hypotrichosis, |
| AS1 | and hyper-Ige |
| DSG2 | Arrhythmogenic right ventricular cardiomyopathy type 10; Cardiac arrest; |
| | Cardiomyopathy; Cardiovascular phenotype; Dilated cardiomyopathy; |
| | Hypertrophic cardiomyopathy |
| DSG2, DSG2- | Dilated cardiomyopathy-1BB |
| AS1 | |
| DSG4, DSG1- | Hypotrichosis-6 |
| AS1 | |
| DSP | Arrhythmogenic right ventricular cardiomyopathy type 8; Arrhythmogenic |
| | right ventricular dysplasia, cardiomyopathy; Cardiac arrest; Dilated |
| | cardiomyopathy with woolly hair, keratoderma, and tooth agenesis; |
| | Cardiovascular phenotype; DSP-related disorders; Dilated cardiomyopathy |
| | with woolly hair and keratoderma; Keratosis palmoplantaris striata-II; Left |
| | ventricular noncompaction cardiomyopathy; Lethal acantholytic |
| | epidermolysis bullosa; Long QT syndrome-1; Primary dilated |
| | cardiomyopathy; Skin fragility-woolly hair-palmoplantar keratoderma |
| | syndrome; Ventricular tachycardia |
| DST | Autosomal recessive epidermolysis bullosa simplex-2; Hereditary sensory |
| | and autonomic neuropathy type VI |
| DUOX2 | Congenital hypothyroidism; Familial thyroid dyshormonogenesis; Inborn |
| | genetic diseases; Nongoitrous euthyroid hyperthyrotropinemia; Thyroid |
| | dyshormonogenesis-6 |
| DVL3 | Autosomal dominant Robinow syndrome-1; Autosomal dominant Robinow |
| | syndrome-3 |
| DYNC2H1 | Jeune thoracic dystrophy; Short-rib polydactyly syndrome; Short-rib |
| | polydactyly syndrome type III; Short-rib thoracic dysplasia-3 with or |
| | without polydactyly |
| DYNC2I1 | Short-rib thoracic dysplasia-8 with or without polydactyly |
| DYNC2I2 | Jeune thoracic dystrophy; Short-rib thoracic dysplasia-11 with or without |
| | polydactyly |
| DYNC2LI1 | Short-rib thoracic dysplasia-15 with polydactyly |
| DYRK1A | Autosomal dominant mental retardation-7 |
| DYSF | Autosomal recessive limb-girdle muscular dystrophy type 2B; Miyoshi |
| | muscular dystrophy-1; Distal myopathy with anterior tibial onset; |
| | Qualitative or quantitative defects of dysferlin |
| ECEL1 | Distal arthrogryposis type 5D; Inborn genetic diseases |
| ECHS1 | Inborn genetic diseases; Mitochondrial short-chain enoyl-CoA hydratase 1 |
| | deficiency |
| ECM1 | Lipid proteinosis |
| EDA | Hypohidrotic X-linked ectodermal dysplasia |
| EDARADD | Autosomal recessive hypohidrotic/hair/tooth type Ectodermal dysplasia-11b |
| EDN3 | Congenital central hypoventilation; Dominant Hirschsprung disease; |
| | Hirschsprung disease; Waardenburg syndrome; Waardenburg syndrome |
| | type 4B |
| EDNRB, | Rare genetic deafness |
| EDNRB-AS1 | |
| EFEMP2 | Autosomal recessive cutis laxa type 1B; Autosomal recessive cutis laxa |
| | type IA |
| EHMT1 | Kleefstra syndrome-1 |
| EIF2AK3 | Wolcott-Rallison dysplasia |
| EIF2AK4 | Autosomal recessive pulmonary venoocclusive disease-2 |
| EIF2B2 | Leukoencephalopathy with vanishing white matter; Ovarioleukodystrophy |
| EIF2S3 | MEHMO syndrome |
| ELN | Inborn genetic diseases; Supravalvar aortic stenosis |
| ELOVL4 | Retinal dystrophy; Stargardt Disease-3 |
| ELP1 | Familial dysautonomia |
| ELP2 | ELP2-related disorders; Autosomal recessive mental retardation-58 |
| EMD | Cardiovascular phenotype; X-linked Emery-Dreifuss muscular dystrophy-1; |
| | Neuromuscular disease |
| ENAM | Amelogenesis imperfecta type IC; Autosomal dominant hypoplastic local |
| | amelogenesis imperfecta |
| ENG | Hereditary hemorrhagic telangiectasia; Hereditary hemorrhagic |
| | telangiectasia type 1 |
| ENG, | Hereditary hemorrhagic telangiectasia type 1 |
| LOC102723566 | |
| EOGT | Adams-Oliver syndrome; Adams-Oliver syndrome-4 |
| EPB42 | Spherocytosis type 5 |
| EPCAM | Congenital diarrhea with tufting enteropathy-5 |
| EPG5 | Vici syndrome |
| EPHB4 | Capillary malformation-arteriovenous malformation-2 |
| EPHB4, | Capillary malformation-arteriovenous malformation-2 |
| SLC12A9 | |
| EPOR | Primary familial polycythemia due to EPO receptor mutation |
| ERCC2 | Metachromatic leukodystrophy variant; Photosensitive trichothiodystrophy- |
| | 1; Xeroderma pigmentosum group D |
| ERCC3 | Complementation group B Xeroderma pigmentosum |
| ERCC4 | Cockayne syndrome; Complementation group Q Fanconi anemia; |
| | Hutchinson-Gilford syndrome; Pre-B-cell acute lymphoblastic leukemia; |
| | XFE progeroid syndrome; Xeroderma pigmentosum group F |
| ERCC5, BIVM- | Xeroderma pigmentosum; group G |
| ERCC5 | |
| ERCC6 | Cerebrooculofacioskeletal syndrome-1; Cockayne syndrome-B; De Sanctis- |
| | Cacchione syndrome |
| ERCC8 | Cockayne syndrome type A |
| ERCC8, | Cockayne syndrome type A |
| ERCC8-AS1 | |
| ERCC8, | Cockayne syndrome type A; Nuclear type 10 mitochondrial complex I |
| NDUFAF2 | deficiency |
| ERF | Craniosynostosis-1; Craniosynostosis-4 |
| ERI1 | Abnormality of finger; Coarse facial features; Global developmental delay; |
| | Unilateral renal agenesis |
| ESCO2 | Roberts-SC phocomelia syndrome |
| ESRP1 | Autosomal recessive deafness-109 |
| ESRRB | Rare genetic deafness |
| ETFDH | Multiple acyl-CoA dehydrogenase deficiency |
| ETHE1 | Ethylmalonic encephalopathy |
| EVC2 | Curry-Hall syndrome; Ellis-van Creveld syndrome |
| EXOSC3 | Pontocerebellar hypoplasia type 1b |
| EXPH5 | Autosomal recessive nonspecific epidermolysis bullosa |
| EXT1 | Sporadic chondrosarcoma; Multiple congenital exostosis; Multiple |
| | exostoses type 1 |
| EXT2 | Multiple exostoses type 2 |
| EYA1 | Branchiootic syndrome; Melnick-Fraser syndrome; Rare genetic deafness |
| EYA4 | Autosomal dominant deafness-10; Dilated cardiomyopathy-1J; Rare genetic |
| | deafness |
| EYA4, TARID | EYA4-related disorders |
| EYS | Retinal dystrophy; Retinitis pigmentosa; Retinitis pigmentosa-25 |
| F13A1 | Factor XIII subunit A deficiency |
| F13B | Factor XIII subunit B deficiency |
| F2 | Congenital prothrombin deficiency |
| F5 | Factor V deficiency |
| F8 | Hereditary factor VIII deficiency disease |
| F9 | Hereditary factor IX deficiency disease; X-linked thrombophilia due to |
| | factor IX defect |
| FA2H | Spastic paraplegia-35 |
| FAH | Tyrosinemia type I |
| FAM161A | Retinal dystrophy; Retinitis pigmentosa; Retinitis pigmentosa-28 |
| FAM20A | Amelogenesis imperfecta type 1G |
| FANCA | Complementation group A Fanconi anemia |
| FANCB | Complementation group B Fanconi anemia |
| FANCC | Complementation group C Fanconi anemia; Hereditary cancer-predisposing |
| | syndrome |
| FANCC, | Complementation group C Fanconi anemia; Hereditary cancer-predisposing |
| AOPEP | syndrome; Tracheoesophageal fistula |
| FANCF | Complementation group F Fanconi anemia |
| FANCM | Fanconi anemia; Malignant germ cell tumor of ovary; Spermatogenic |
| | failure-28 |
| FARS2 | Combined oxidative phosphorylation deficiency-14 |
| FARSB | Interstitial lung and liver disease; Rajab interstitial lung disease with brain |
| | calcifications |
| FAS | Autoimmune lymphoproliferative syndrome |
| FAT4 | Van Maldergem syndrome |
| FBN1 | Acromicric dysplasia; Acute aortic dissection; Cardiovascular phenotype; |
| | Autosomal dominant isolated ectopia lentis; Familial thoracic aortic |
| | aneurysm; Familial thoracic aortic aneurysm and aortic dissection; |
| | Geleophysic dysplasia-2; Inborn genetic diseases; MASS syndrome; Marfan |
| | syndrome, Loeys-Dietz syndrome, Familial thoracic aortic aneurysms and |
| | dissections; Marfan lipodystrophy syndrome; Marfan syndrome; Stiff skin |
| | syndrome; Weill-Marchesani syndrome-2 |
| FBN1, | Marfan syndrome; Loeys-Dietz syndrome; Familial thoracic aortic |
| LOC113939944 | aneurysms and dissections; Marfan syndrome |
| FBXL4 | Inborn genetic diseases; Mitochondrial DNA depletion syndrome; |
| | Encephalomyopathic type mitochondrial DNA depletion syndrome-13 |
| FERMT1 | Kindler's syndrome |
| FEZF1-AS1, | Hypogonadotropic hypogonadism-22 with anosmia |
| FEZF1 | |
| FGD4 | Charcot-Marie-Tooth disease; Charcot-Marie-Tooth disease type 4 |
| FGF16 | Metacarpal 4-5 fusion |
| FGF3 | Deafness with labyrinthine aplasia microtia and microdontia (LAMM) |
| FGG | Congenital afibrinogenemia; Hypofibrinogenemia |
| FH | Fumarase deficiency; Hereditary cancer-predisposing syndrome; Hereditary |
| | leiomyomatosis and renal cell cancer |
| FIG4 | Charcot-Marie-Tooth disease type 4J; Charcot-Marie-Tooth disease type 4; |
| | Yunis-Varon syndrome |
| FKBP10 | Bruck syndrome-1; Osteogenesis imperfecta type 12 |
| FKBP14, | Congenital muscular dystrophy; Ehlers-Danlos syndrome with progressive |
| FKBP14-AS1 | kyphoscoliosis, hearing loss, myopathy; Inborn genetic diseases; Joint |
| | hypermobility; Muscular hypotonia; Pes valgus; Thoracolumbar scoliosis |
| FKRP | Limb-girdle muscular dystrophy-dystroglycanopathy type C5 |
| FKTN | Congenital muscular dystrophy-dystroglycanopathy with brain and eye |
| | anomalies type A4; Congenital muscular dystrophy-dystroglycanopathy |
| | without mental retardation type B4; FKTN-related disorders; Fukuyama |
| | congenital muscular dystrophy; Limb-girdle muscular dystrophy- |
| | dystroglycanopathy type C4; Walker-Warburg congenital muscular |
| | dystrophy |
| FLCN | Hereditary cancer-predisposing syndrome; Multiple fibrofolliculomas; |
| | Primary spontaneous pneumothorax |
| FLG | Atopic dermatitis-2; FLG-related disorder; Ichthyosis vulgaris |
| FLNA | Periventricular nodular heterotopia-1 |
| FLNB | Spondylocarpotarsal synostosis syndrome |
| FLNC | Familial hypertrophic cardiomyopathy-26; Dilated Cardiomyopathy; |
| | Dominant filamin C-related myofibrillar myopathy; Distal myopathy-4 |
| FLNC, FLNC- | Familial hypertrophic cardiomyopathy-26; Dilated Cardiomyopathy; |
| AS1 | Dominant filamin C-related myofibrillar myopathy; Distal myopathy-4 |
| FLT4 | Congenital heart defects-7 |
| FMR1 | Intellectual disability |
| FOXF1 | Persistent fetal circulation syndrome |
| FOXG1 | History of neurodevelopmental disorder; Congenital variant Rett syndrome |
| FOXL2 | Blepharophimosis, ptosis, epicanthus inversus type 1 |
| FOXN1 | Autosomal dominant infantile T-cell lymphopenia with or without nail |
| | dystrophy; T-cell immunodeficiency, congenital alopecia, and nail |
| | dystrophy |
| FOXP1 | Mental retardation with language impairment and with or without autistic |
| | features |
| FOXRED1 | Leigh syndrome; Nuclear type 1 mitochondrial complex I deficiency |
| FRAS1 | Fraser syndrome-1 |
| FREM2 | Isolated unilateral or bilateral cryptophthalmos; Fraser syndrome-2; Fraser |
| | syndrome-1 |
| FSHB | Hypogonadotropic hypogonadism-24 without anosmia |
| FSIP2 | Spermatogenic failure-34 |
| FSIP2, FSIP2- | Spermatogenic failure-34 |
| AS1 | |
| FTCD | Glutamate formiminotransferase deficiency |
| FTSJ1 | X-linked mental retardation-9 |
| FUCA1 | Fucosidosis |
| FYCO1 | Cataract-18 |
| FZD4, PRSS23 | Exudative retinopathy; Familial exudative vitreoretinopathy |
| G6PC | Glycogen storage disease; Glycogen storage disease due to glucose-6- |
| | phosphatase deficiency type IA |
| GAA | Glycogen storage disease type II; Pompe disease |
| GABRA1 | Juvenile myoclonic epilepsy-5; Early infantile epileptic encephalopathy-19 |
| GABRA6 | GABRA6-related disorder |
| GALC | Galactosylceramide beta-galactosidase deficiency |
| GALM | Galactosemia-IV |
| GALNS | MPS-IV-A mucopolysaccharidosis; Morquio syndrome |
| GALT | Deficiency of UDPglucose-hexose-1-phosphate uridylyltransferase |
| GAMT | Cerebral creatine deficiency syndrome; Deficiency of guanidinoacetate |
| | methyltransferase |
| GAREM2, | Long-chain 3-hydroxyacyl-CoA dehydrogenase deficiency; Mitochondrial |
| HADHA | trifunctional protein deficiency |
| GATA1 | Acute megakaryoblastic leukemia |
| GATA3 | Hypoparathyroidism-deafness-renal disease syndrome |
| GATA6 | Abnormality of cardiovascular system morphology; Congenital |
| | diaphragmatic hernia; Pancreatic agenesis and congenital heart disease; |
| | Persistent truncus arteriosus |
| GATAD1, PEX1 | Deafness, enamel hypoplasia, and nail defects; Zellweger peroxisome |
| | biogenesis disorder 1A |
| GATAD2B | GATAD2B-related disorder; Autosomal dominant mental retardation-18 |
| GBA | Acute neuronopathic Gaucher's disease; Gaucher disease; Gaucher disease |
| | type 3C; Gaucher's disease type 1; Subacute neuronopathic Gaucher's |
| | disease |
| GBA, | Gaucher disease type 1; Perinatal lethal Gaucher's disease |
| LOC106627981 | |
| GBE1 | Classic hepatic glycogen storage disease type IV; Fatal perinatal |
| | neuromuscular glycogen storage disease-IV |
| GCDH | Glutaric aciduria type 1 |
| GCH1 | Dystonia-5 |
| GCK | Maturity onset diabetes mellitus in young; Maturity-onset diabetes of the |
| | young type 2 |
| GDAP1 | Recessive intermediate Charcot-Marie-Tooth disease type 4A |
| GDF1, CERS1 | Heterotaxia |
| GDF9 | Premature ovarian failure-14 |
| GFER | Mitochondrial diseases |
| GHR | Laron syndrome with elevated serum GH-binding protein; Laron-type |
| | isolated somatotropin defect |
| GJB1 | Charcot-Marie-Tooth Neuropathy-X; Charcot-Marie-Tooth disease |
| GJB2 | Bilateral conductive hearing impairment; Bilateral sensorineural hearing |
| | impairment; GJB2-related disorders; Dominant digenic deafness- |
| | GJB2/GJB3; Digenic deafness-GJB2/GJB6; Hearing impairment; |
| | Autosomal dominant hearing loss-3A; Hystrix-like ichthyosis with |
| | deafness; Inborn genetic diseases; Autosomal dominant keratitis ichthyosis |
| | and deafness syndrome; Recessive keratitis-ichthyosis-deafness syndrome; |
| | Knuckle pads, deafness, and leukonychia syndrome; Mutilating |
| | keratoderma; Autosomal recessive nonsyndromic hearing loss-1A; |
| | Nonsyndromic hearing loss and deafness; Palmoplantar keratoderma- |
| | deafness syndrome; Rare genetic deafness |
| GJB3 | Autosomal dominant deafness-2B |
| GLA, RPL36A- | Fabry disease |
| HNRNPH2 | |
| GLB1 | GLB1-related disorders; GM1 gangliosidosis; GM1 gangliosidosis type 2; |
| | GM1 gangliosidosis type 3; GM1-gangliosidosis type I with cardiac |
| | involvement; Infantile GM1 gangliosidosis; MPS-IV- |
| | Bmucopolysaccharidosis |
| GLDC | Non-ketotic hyperglycinemia |
| GLDN | Lethal congenital contracture syndrome-11 |
| GLI3 | Greig cephalopolysyndactyly syndrome; Pallister-Hall syndrome; Postaxial |
| | polydactyly type A1, B; Preaxial polydactyly-4 |
| GLIS3 | Neonatal diabetes mellitus with congenital hypothyroidism |
| GLMN | Glomuvenous malformations |
| GLRA1 | Hyperekplexia-1 |
| GNAS | Progressive osseous heteroplasia; Pseudohypoparathyroidism; |
| | Pseudopseudohypoparathyroidism |
| GNAT2 | Achromatopsia-4 |
| GNB5 | Intellectual developmental disorder with cardiac arrhythmia; Language |
| | delay and attention deficit-hyperactivity disorder, cognitive impairment |
| | with or without cardiac arrhythmia |
| GNPAT | Rhizomelic chondrodysplasia punctata type 2 |
| GNPTAB | GNPTAB-related disorders; Inborn genetic diseases; Mucolipidosis-III |
| | Alpha, Beta; Mucolipidosis; Mucolipidosis type II; Pseudo-Hurler |
| | polydystrophy |
| GNPTG | Mucolipidosis; Mucolipidosis type III Gamma |
| GORAB | Geroderma osteodysplastica |
| GOSR2, | Progressive myoclonic epilepsy |
| LRRC37A2 | |
| GPC3 | Simpson-Golabi-Behmel syndrome; Wilms tumor-1 |
| GPC4 | Keipert syndrome |
| GPC6 | Autosomal recessive omodysplasia |
| GPC6, GPC6- | Autosomal recessive omodysplasia |
| AS2 | |
| GPI | Nonspherocytic hemolytic anemia due to glucose phosphate isomerase |
| | deficiency |
| GPNMB | Primary localized cutaneous amyloidosis-3 |
| GPR143 | Ocular albinism type I |
| GPR179 | Congenital stationary night blindness type 1E; Retinal dystrophy |
| GPSM2 | Chudley-Mccullough syndrome; GPSM2-related disorders; Rare genetic |
| | deafness |
| GRHL2 | Autosomal dominant deafness-28 |
| GRHL3 | Van der Woude syndrome-2 |
| GRHPR | Nephrocalcinosis; Nephrolithiasis; Primary hyperoxaluria type II |
| GRIN2B | Autosomal dominant mental retardation-6 |
| GRIP1 | Fraser syndrome-3 |
| GRN | Frontotemporal dementia |
| GRXCR1 | Autosomal recessive deafness-25; Rare genetic deafness |
| GSDME | Autosomal dominant deafness-5 |
| GUCY2C, | Meconium ileus |
| C12orf60 | |
| GUSB | Mucopolysaccharidosis type 6; Mucopolysaccharidosis type 7 |
| GYG1 | Glycogen storage disease XV; Polyglucosan body myopathy-2 |
| GYS1 | Muscle glycogen storage disease-0 |
| GYS2 | Glycogen storage disease; Glycogen storage disease due to hepatic |
| | glycogen synthase deficiency |
| GZF1 | Joint laxity, short stature, and myopia |
| H1-4 | Inborn genetic diseases; Rahman syndrome |
| H6PD | Cortisone reductase deficiency-1 |
| HADHA | HADHA-related disorders; Long-chain 3-hydroxyacyl-CoA dehydrogenase |
| | deficiency; Mitochondrial trifunctional protein deficiency |
| HADHA, | HADHA-related disorders; Inborn genetic diseases; LCHAD deficiency; |
| GAREM2 | LCHAD deficiency with maternal acute fatty liver of pregnancy; Long- |
| | chain 3-hydroxyacyl-CoA dehydrogenase deficiency; Mitochondrial |
| | trifunctional protein deficiency |
| HAX1 | Autosomal recessive severe congenital neutropenia-3 |
| HBA2, | Alpha plus thalassemia |
| LOC106804612 | |
| HBB, | Anemia; Beta thalassemia major; Beta-plus-thalassemia; Beta-thalassemia; |
| LOC106099062, | Familial erythrocytosis-6; Fetal hemoglobin quantitative trait locus 1; HBB- |
| LOC107133510 | related disorders; Hb SS disease; Heinz body anemia; Hemoglobin E; |
| | Hemoglobin E disease; Hemoglobin E, Beta thalassemia disease; |
| | Hemoglobin M disease; Hemoglobinopathy; Resistance to malaria; |
| | Susceptibility to malaria; Alpha thalassemia; Dominant inclusion body type |
| | beta thalassemia |
| HBB, | Beta thalassemia |
| LOC107133510, | |
| LOC110006319 | |
| HCN4 | Brugada syndrome-8; Autosomal dominant sick sinus syndrome-2 |
| HEXA | Inborn genetic diseases; Tay-Sachs disease |
| HEXB | Infantile Sandhoff disease |
| HFM1 | Premature ovarian failure-9 |
| HGD | Alkaptonuria |
| HGSNAT | MPS-III-C mucopolysaccharidosis; Retinitis pigmentosa-73; Sanfilippo |
| | syndrome |
| HIVEP2 | Angelman syndrome-like; Autosomal dominant mental retardation-43 |
| HJV | Hemochromatosis type 2A |
| HLCS | Holocarboxylase synthetase deficiency |
| HMCN1 | Age-related macular degeneration-1 |
| HMGB3 | Syndromic microphthalmia-13 |
| HMGCL | Deficiency of hydroxymethylglutaryl-CoA lyase |
| HNF1A | Clear cell carcinoma of kidney; Insulin-dependent diabetes mellitus; Type 1 |
| | diabetes mellitus-20; Familial hepatic adenomas; Maturity onset diabetes |
| | mellitus in young; Maturity-onset diabetes of the young type 3 |
| HNF1B | Familial hypoplastic renal cysts and diabetes syndrome; Glomerulocystic |
| | kidney |
| HNRNPK | Au-Kline syndrome |
| HNRNPU | Epileptic encephalopathy |
| HOXA1 | Athabaskan brainstem dysgenesis syndrome; Bosley-Salih-Alorainy |
| | syndrome |
| HOXA11 | Radioulnar synostosis with amegakaryocytic thrombocytopenia-1 |
| HOXD13 | Synpolydactyly-1 |
| HPGD | HPGD-related disorders; Autosomal recessive primary hypertrophic |
| | osteoarthropathy-1 |
| HPS1 | Hermansky-Pudlak syndrome; Hermansky-Pudlak syndrome-1 |
| HPS5 | Hermansky-Pudlak syndrome; Hermansky-Pudlak syndrome-5 |
| HPS6 | Hermansky-Pudlak syndrome; Hermansky-Pudlak syndrome-6 |
| HPSE2 | Urofacial syndrome-1 |
| HR | Atrichia with papular lesions |
| HSD17B10 | HSD10 disease |
| HSD17B4 | Bifunctional peroxisomal enzyme deficiency; Perrault syndrome |
| HSPA9 | Even-plus syndrome; Sideroblastic anemia-4 |
| HSPB1 | Charcot-Marie-Tooth disease; Charcot-Marie-Tooth disease axonal type 2F; |
| | Distal hereditary motor neuronopathy type 2B |
| HSPG2 | Lethal Kniest-like syndrome; Schwartz-Jampel syndrome |
| HYAL1 | Deficiency of hyaluronoglucosaminidase |
| HYDIN | Primary ciliary dyskinesia-5 |
| ICAM4 | Landsteiner-Wiener phenotype |
| DDS | MPS-II mucopolysaccharidosis |
| IDS, | MPS-II mucopolysaccharidosis |
| LOC106050102 | |
| IDUA | Hurler syndrome; MPS-I-H/S mucopolysaccharidosis; MPS-I-S |
| | mucopolysaccharidosis type 1 |
| IDUA, | Hurler syndrome; MPS-I-H/S mucopolysaccharidosis; MPS-I-S |
| SLC26A1 | mucopolysaccharidosis type 1 |
| IFIH1 | Aicardi-Goutieres syndrome-7; Singleton-Merten syndrome-1 |
| IFNGR1 | Disseminated atypical mycobacterial infection; IFN-gamma receptor 1 |
| | deficiency; Immunodeficiency-27B; Inherited immunodeficiency diseases |
| IFNGR2 | Immunodeficiency-28 |
| IFT140 | Retinitis pigmentosa-80 |
| IFT140, | Jeune thoracic dystrophy; Joubert syndrome with Jeune asphyxiating |
| LOC105371046 | thoracic dystrophy; Renal dysplasia, cerebellar ataxia, skeletal dysplasia, |
| | and retinal pigmentary dystrophy |
| IFT172 | Short-rib thoracic dysplasia-10 with or without polydactyly |
| IFT52 | Short Rib Polydactyly Syndrome; Short-rib thoracic dysplasia-16 with or |
| | without polydactyly |
| IGF1 | Growth delay due to insulin-like growth factor type 1 deficiency |
| IGF1R | Inborn genetic diseases |
| IGFALS | Acid-labile subunit deficiency |
| IGHM | Non-Bruton type agammaglobulinemia |
| IGHMBP2 | Autosomal dominant distal hereditary motor neuropathy; Charcot-Marie- |
| | Tooth disease axonal type 2S; Distal spinal muscular atrophy; Inborn |
| | genetic diseases; Autosomal recessive distal spinal muscular atrophy-1 |
| IGLL1 | Autosomal recessive agammaglobulinemia-2 |
| IGSF1 | Central hypothyroidism and testicular enlargement |
| IGSF3 | Lacrimal duct defect |
| IKBKG | Ectodermal dysplasia and immunodeficiency-1; Immunodeficiency without |
| | anhidrotic ectodermal dysplasia; Incontinentia pigmenti |
| IL12B | Immunodeficiency-29 |
| IL12RB1 | Immunodeficiency-30 |
| IL2RB | Ichthyosis |
| IL2RG | X-linked combined immunodeficiency; X-linked severe combined |
| | immunodeficiency |
| IL36RN | Generalized pustular psoriasis |
| IL7R | Autosomal recessive T cell-negative, B cell-positive, NK cell-positive |
| | severe combined immunodeficiency |
| INPP5E | Retinal dystrophy |
| INPPL1 | Opsismodysplasia |
| INTU | Mohr syndrome; Orofaciodigital syndrome-17 |
| IQCB1 | Renal dysplasia and retinal aplasia |
| IQCE | Postaxial polydactyly type A7 |
| IQSEC2 | Mental retardation; X-linked severe intellectual deficiency-1 |
| IRAK4 | Immunodeficiency due to interleukin-1 receptor-associated kinase-4 |
| | deficiency |
| IRF2BPL | Neurodevelopmental disorder; Neurodevelopmental disorder with |
| | regression, abnormal movements, loss of speech, and seizures |
| IRF6 | Van der Woude syndrome |
| IRS4 | Congenital nongoitrous hypothyroidism-9 |
| ISCA2 | Multiple mitochondrial dysfunctions syndrome-4 |
| ISG15 | Immunodeficiency-38 with basal ganglia calcification |
| ITGA7 | Congenital muscular dystrophy due to integrin alpha-7 deficiency |
| ITGB2 | Leukocyte adhesion deficiency |
| ITGB4 | Epidermolysis bullosa junctionalis with pyloric atresia |
| ITPA | Early infantile epileptic encephalopathy-35; Inosine triphosphatase |
| | deficiency |
| ITPR1 | Gillespie syndrome |
| IVD | Isovaleric acidemia; Isovaleryl-CoA dehydrogenase deficiency type III |
| JAG1 | Alagille syndrome-1; Arteriohepatic dysplasia; Malformation of heart |
| JAK3 | Autosomal recessive T cell-negative, B cell-positive, NK cell-negative |
| | severe combined immunodeficiency; Severe combined immunodeficiency |
| | disease |
| KAT6A | History of neurodevelopmental disorder; Autosomal dominant mental |
| | retardation-32 |
| KAT6B | SBBYS type blepharophimosis-intellectual disability syndrome |
| KAT6B, DUPD1 | SBBYS type blepharophimosis-intellectual disability syndrome; |
| | Genitopatellar syndrome; Inborn genetic diseases |
| KATNIP | Joubert syndrome-26 |
| KCNA1 | Episodic ataxia type 1 |
| KCNA5 | Familial atrial fibrillation-7 |
| KCNC1 | Progressive myoclonic epilepsy-7 |
| KCNE1 | Long QT syndrome |
| KCNH2 | Cardiac arrhythmia; Cardiovascular phenotype; Congenital long QT |
| | syndrome; Long QT syndrome; Digenic long QT syndrome-1/2; Long QT |
| | syndrome-2 |
| KCNK18 | Migraine with or without aura-13 |
| KCNQ1 | Cardiac arrhythmia; Cardiovascular phenotype; Congenital long QT |
| | syndrome; Jervell and Lange-Nielsen syndrome; Jervell and Lange-Nielsen |
| | syndrome-1; KCNQ1-related disorders; Long QT syndrome LQT1 subtype; |
| | Long QT syndrome-1; Rare genetic deafness; Romano-Ward syndrome |
| KCNQ1-AS1, | Jervell and Lange-Nielsen syndrome-1 |
| KCNQ1 | |
| KCNQ1, | Cardiovascular phenotype; Long QT syndrome; Long QT syndrome-1 |
| KCNQ1-AS1 | |
| KCNQ1, | Congenital long QT syndrome; Long QT syndrome LQT1 subtype |
| KCNQ1OT1 | |
| KCNQ2 | Benign familial neonatal seizures-1; Early infantile epileptic |
| | encephalopathy; Early infantile epileptic encephalopathy-7; Epileptic |
| | encephalopathy; Inborn genetic diseases; Seizures |
| KCNQ3 | Intellectual disability; Seizures |
| KCNQ4 | Autosomal dominant nonsyndromic deafness-2A |
| KCNT1 | Early infantile epileptic encephalopathy-14; Nocturnal frontal lobe |
| | epilepsy-5 |
| KCNV2 | Cone dystrophy with supernormal rod response; Progressive cone dystrophy |
| | without rod involvement; Retinal dystrophy; Stargardt disease |
| KDM5B | Autosomal recessive intellectual disability-65 |
| KDM5C | X-linked Claes-Jensen type syndromic mental retardation |
| KDM6A | Kabuki syndrome-2 |
| KERA | Cornea plana-2 |
| KHDC3L | Recurrent hydatidiform mole-2 |
| KIAA0586 | Congenital cerebellar hypoplasia; Intellectual disability; Joubert syndrome; |
| | Joubert syndrome-23; Retinal dystrophy; Rod-cone dystrophy; Short-rib |
| | thoracic dysplasia-14 with polydactyly |
| KIAA0753 | Orofaciodigital syndrome-XV |
| KIAA0825 | Postaxial polydactyly type A10; Postaxial polydactyly type A1 |
| KIAA1549 | Retinitis pigmentosa-86 |
| KIF11 | Microcephaly with or without chorioretinopathy, lymphedema, and mental |
| | retardation |
| KIF7 | Acrocallosal syndrome; Joubert syndrome-12 |
| KIFBP | Goldberg-Shprintzen megacolon syndrome |
| KISSIR | Hypogonadotropic hypogonadism-8 without anosmia |
| KIZ | Retinitis pigmentosa-69 |
| KMT2A | Wiedemann-Steiner syndrome |
| KMT2B | Childhood-onset dystonia-28 |
| KMT2C | Kleefstra syndrome due to a point mutation |
| KMT2D | CHARGE association; Kabuki syndrome; Kabuki syndrome-1 |
| KMT2E | Epilepsy; Leukoencephalopathy; Macrocephalus; O'Donnell-Luria-Rodan |
| | syndrome; Intellectual deficiency |
| KPTN | Autosomal recessive mental retardation-41 |
| KRIT1 | Cavernous malformations of CNS and retina; Cerebral cavernous |
| | malformation; Cerebral cavernous malformations-1 |
| KRT1 | Curth-Macklin type ichthyosis histrix; |
| KRT10 | Bullous ichthyosiform erythroderma |
| KRT10, | Bullous ichthyosiform erythroderma |
| TMEM99 | |
| KRT14 | Autosomal recessive epidermolysis bullosa simplex |
| KRT5 | Dowling-Degos disease-1 |
| KRT6A | Pachyonychia congenita-3 |
| KRT85 | ‘Pure’ hair-nail type ectodermal dysplasia |
| KYNU | Congenital NAD deficiency disorder; Vertebral, cardiac, renal, and limb |
| | defects syndrome-2 |
| L1CAM | MASA syndrome; Spastic paraplegia |
| L2HGDH | L-2-hydroxyglutaric aciduria |
| LACC1 | Juvenile arthritis |
| LAMA2 | Inborn genetic diseases; Laminin alpha 2-related dystrophy; Merosin |
| | deficient congenital muscular dystrophy |
| LAMA3 | Junctional epidermolysis bullosa gravis of Herlitz |
| LAMA4 | Dilated cardiomyopathy-1JJ |
| LAMB3 | Amelogenesis imperfecta type 1A; Non-Herlitz type junctional |
| | epidermolysis bullosa; Junctional epidermolysis bullosa gravis of Herlitz |
| LAMC2 | Non-Herlitz type junctional epidermolysis bullosa; Junctional epidermolysis |
| | bullosa gravis of Herlitz |
| LAMP2 | Cardiomyopathy; Danon disease; Hypertrophic cardiomyopathy; Primary |
| | dilated cardiomyopathy |
| LARGE1 | Congenital muscular dystrophy-dystroglycanopathy with mental retardation |
| | type B6 |
| LBR | Disproportionate short stature; Femoral bowing; Pelger-Huet anomaly; |
| | Regressive spondylometaphyseal dysplasia; Retrognathia; Rhizomelic arm |
| | shortening; Rhizomelic leg shortening; Short long bone |
| LDB3 | Cardiomyopathy; ZASP-related myofibrillar myopathy |
| LDLR | Familial hypercholesterolemia; Familial hypercholesterolemia-1; |
| | Homozygous familial hypercholesterolemia |
| LDLRAP1 | Familial hypercholesterolemia-4 |
| LEP | Leptin deficiency or dysfunction |
| LFNG | Autosomal recessive spondylocostal dysostosis-3 |
| LGI1 | Familial temporal lobe epilepsy-1 |
| LHFPL5 | Rare genetic deafness |
| LHX3 | Non-acquired combined pituitary hormone deficiency with spine |
| | abnormalities |
| LIFR | Stuve-Wiedemann syndrome |
| LIG4 | LIG4-related disorders; Lig4 syndrome |
| LIPA | Lysosomal acid lipase deficiency |
| LIPE | Familial partial lipodystrophy-6 |
| LIPE, LIPE- | Familial partial lipodystrophy-6 |
| AS1, | |
| LOC101930071 | |
| LIPH | Hypotrichosis-7; Autosomal recessive woolly hair-2 with or without |
| | hypotrichosis |
| LIPN | Autosomal recessive congenital ichthyosis-8 |
| LMBR1 | Acheiropodia |
| LMBRD1 | Inborn genetic diseases; Methylmalonic aciduria and homocystinuria type |
| | cb1F |
| LMNA | Cardiovascular phenotype; Charcot-Marie-Tooth disease type 2; Primary |
| | dilated cardiomyopathy |
| LMOD3 | Nemaline myopathy-10 |
| LMX1B | Nail-patella syndrome |
| LOC100507346, | Gorlin syndrome; Medulloblastoma |
| PTCH1 | |
| LOC101927055, | Dilated cardiomyopathy 1G; Limb-girdle muscular dystrophy type 2J; |
| TTN | Primary dilated cardiomyopathy |
| LOC101927157, | Retinitis pigmentosa; Retinitis pigmentosa-49 |
| CNGA1 | |
| LOC101927188, | Poretti-Boltshauser syndrome |
| LAMA1 | |
| LOC102723566, | Hereditary hemorrhagic telangiectasia type 1 |
| ENG | |
| LOC106694316, | Myeloperoxidase deficiency |
| MPO | |
| LOC110006319, | Beta thalassemia |
| HBB, | |
| LOC107133510 | |
| LOXHD1 | Autosomal recessive deafness-77; Rare genetic deafness |
| LPL | Hyperlipoproteinemia type I |
| LRAT | Leber congenital amaurosis; Leber congenital amaurosis-14; Early-onset |
| | severe retinal dystrophy; Juvenile LRAT-related retinitis pigmentosa |
| LRBA | Common variable immunodeficiency-8 with autoimmunity |
| LRIT3 | Congenital stationary night blindness type 1F |
| LRP4 | Cenani-Lenz syndactyly syndrome |
| LRP5 | Exudative vitreoretinopathy-4; Autosomal dominant familial exudative |
| | vitreoretinopathy |
| LRP6 | Selective tooth agenesis-7 |
| LRPAP1 | Autosomal recessive myopia-23; Rare isolated myopia |
| LRPPRC | Saguenay-Lac-Saint-Jean type congenital lactic acidosis |
| LRSAM1 | Charcot-Marie-Tooth disease type 2P |
| LRTOMT | Autosomal recessive deafness-63; Rare genetic deafness |
| LTBP2 | Congenital glaucoma; Microspherophakia |
| LTBP3 | Dental anomalies and short stature |
| LTBP4 | Cutis laxa with severe pulmonary, gastrointestinal, and urinary |
| | abnormalities |
| LYRM7 | Nuclear type 8 Mitochondrial complex III deficiency |
| LZTFL1 | Bardet-Biedl syndrome-17 |
| LZTR1 | Noonan syndrome-2; Schwannomatosis-2 |
| MAB21L1, | Cerebellar, ocular, craniofacial, and genital syndrome |
| NBEA | |
| MAFB | Duane retraction syndrome-2; Duane retraction syndrome-3 with or without |
| | deafness; Duane syndrome type 1; Duane syndrome type 3 |
| MAGED2 | Transient antenatal Bartter syndrome type 5 |
| MAGEL2 | Inborn genetic diseases; Schaaf-Yang syndrome |
| MAGT1 | X-Linked immunodeficiency with magnesium defect, Epstein-Barr virus |
| | infection, and neoplasia |
| MAK | Retinal dystrophy |
| MAN2B1 | Deficiency of alpha-mannosidase |
| MANBA | Beta-D-mannosidosis |
| MAP2K2 | Rasopathy |
| MAPRE2 | Congenital symmetric circumferential skin creases-2 |
| MARVELD2 | Autosomal recessive neurosensory deafness-49; Rare genetic deafness |
| MAX | Hereditary cancer-predisposing syndrome |
| MBD5 | Autosomal dominant mental retardation-1 |
| MC2R | ACTH resistance |
| MC4R | Monogenic diabetes; Obesity; Schizophrenia |
| MCCC1 | Methylcrotonyl-CoA carboxylase 1 deficiency-3 |
| MCCC2 | Methylcrotonyl CoA carboxylase 2 deficiency-3 |
| MCM5 | Meier-Gorlin syndrome-8 |
| MCM8 | Premature ovarian failure-10 |
| MCOLN1 | Mucolipidosis type IV |
| MCPH1 | Abnormality of brain morphology; Primary autosomal recessive |
| | microcephaly-1 |
| MECP2 | Angelman syndrome; Atypical Rett syndrome; X-linked susceptibility to |
| | autism-3; Delayed gross motor development; Delayed speech and language |
| | development; Developmental regression; Encephalopathy; Global |
| | developmental delay; History of neurodevelopmental disorder; Inborn |
| | genetic diseases; Intellectual disability; Loss of ability to walk; X-linked |
| | syndromic mental retardation-13; Rett syndrome; Severe neonatal-onset |
| | encephalopathy with microcephaly, Smith-Magenis Syndrome-like; Lubs |
| | type syndromic X-linked intellectual disability; Neonatal severe mental |
| | retardation; Syndromic Rett syndrome-13 |
| MED12 | Cardiovascular phenotype; FG syndrome-1; History of neurodevelopmental |
| | disorder |
| MED13L | Mental retardation and distinctive facial features with or without cardiac |
| | defects |
| MED25 | Broad-based gait; Charcot-Marie-Tooth disease type 2; Decreased body |
| | weight; Failure to thrive; Generalized hypotonia; Impaired distal |
| | proprioception; Sensory ataxia; Sensory ataxic neuropathy; Sensory |
| | neuropathy |
| MEF2C | MEF2C-related disorder; Mental retardation, epilepsy, and stereotypic |
| | movements and/or cerebral malformations |
| MEFV | Familial Mediterranean fever |
| MEN1 | Hereditary cancer-predisposing syndrome; Somatic lipoma; Multiple |
| | endocrine neoplasia type 1 |
| MERTK | Retinitis pigmentosa-38 |
| MESD | Osteogenesis imperfecta type XX |
| METTL23 | Inborn genetic diseases; Autosomal recessive mental retardation-44 |
| MFN2 | Charcot-Marie-Tooth disease type 2 |
| MFRP, | Isolated microphthalmia-5; Nanophthalmos-2 |
| C1QTNF5 | |
| MFSD8 | Neuronal ceroid lipofuscinosis-7 |
| MIP | Cataract-15 (multiple types) |
| MIR6886, | Familial hypercholesterolemia; Familial hypercholesterolemia-1; |
| LDLR | Homozygous familial hypercholesterolemia |
| MITF | Coloboma, osteopetrosis, microphthalmia, macrocephaly, albinism, and |
| | deafness; Rare genetic deafness; Waardenburg syndrome type 2A |
| MKRN3 | Central precocious puberty-2 |
| MKS1 | Joubert syndrome; Joubert syndrome-28; Meckel syndrome type 1; Meckel- |
| | Gruber syndrome |
| MLC1 | Megalencephalic leukoencephalopathy with subcortical cysts-1 |
| MLH1 | Carcinoma of colon; Colon cancer; Hereditary cancer-predisposing |
| | syndrome; Hereditary nonpolyposis colon cancer; Lynch syndrome; Lynch |
| | syndrome-I; Lynch syndrome-II; Muir-Torre syndrome; Turcot syndrome |
| MLH3 | Hereditary nonpolyposis colorectal cancer type 7 |
| MLYCD | Deficiency of malonyl-CoA decarboxylase |
| MMAA | Methylmalonic acidemia; Vitamin B12-responsive methylmalonic acidemia |
| | type cblA |
| MMAB | Methylmalonic acidemia; Vitamin B12-responsive methylmalonic acidemia |
| | type cblB |
| MMACHC | Disorders of intracellular cobalamin metabolism; Digenic methylmalonic |
| | aciduria and homocystinuria clbC type; Methylmalonic acidemia with |
| | homocystinuria; Methylmalonic aciduria due to methylmalonyl-CoA |
| | mutase deficiency |
| MME | Charcot-Marie-Tooth disease axonal type 2T; Congenital membranous |
| | nephropathy due to fetomaternal anti-neutral endopeptidase |
| | alloimmunization |
| MMUT | Methylmalonic acidemia; Methylmalonic aciduria due to methylmalonyl- |
| | CoA mutase deficiency |
| MOCS2 | Complementation group B molybdenum cofactor deficiency |
| MPDZ | Congenital hydrocephalus; Congenital hydrocephalus-2 with or without |
| | brain or eye anomalies |
| MPL | Congenital amegakaryocytic thrombocytopenia; Essential thrombocytemia |
| MPLKIP | Nonphotosensitive trichothiodystrophy-1 |
| MPO | Myeloperoxidase deficiency |
| MPV17 | Navajo neurohepatopathy |
| MPZ | Charcot-Marie-Tooth disease |
| MPZL2 | Autosomal recessive deafness-111 |
| MRE11 | Hereditary cancer-predisposing syndrome |
| MSH2 | Carcinoma of colon; Colon cancer; Glioblastoma; Hereditary cancer- |
| | predisposing syndrome; Hereditary nonpolyposis colon cancer; Lynch |
| | syndrome; Lynch syndrome-I; Malignant tumor of ascending colon; |
| | Malignant tumor of sigmoid colon; Muir-Torre syndrome; Ovarian |
| | neoplasms; Turcot syndrome |
| MSH6 | Endometrial carcinoma; Hereditary cancer-predisposing syndrome; |
| | Hereditary nonpolyposis colon cancer; Hereditary nonpolyposis colorectal |
| | cancer type 5; Hereditary nonpolyposis colorectal carcinoma; Lynch |
| | syndrome; Lynch syndrome-I; Turcot syndrome |
| MSTO1 | Mitochondrial myopathy-cerebellar ataxia-pigmentary retinopathy |
| | syndrome |
| MSX2 | Parietal foramina-1 |
| MTFMT | Abnormal facial shape; Combined oxidative phosphorylation deficiency-15; |
| | Cytochrome C oxidase-negative muscle fibers; Decreased activity of |
| | mitochondrial complex I; Inability to walk by childhood/adolescence; Leigh |
| | syndrome; Nuclear type 27 Mitochondrial complex I deficiency; |
| | Mitochondrial oxidative phosphorylation disorder; Poor speech; Short |
| | stature |
| MTHFD1 | Combined immunodeficiency and megaloblastic anemia with or without |
| | hyperhomocysteinemia |
| MTM1 | Severe X-linked myotubular myopathy |
| MTRR | Disorders of intracellular cobalamin metabolism; Homocystinuria without |
| | methylmalonic aciduria; CblE complementation type homocystinuria- |
| | megaloblastic anemia due to defect in cobalamin metabolism |
| MTTP | Abetalipoproteinaemia |
| MUTYH | Carcinoma of colon; Colon cancer; Familial colorectal cancer; Hereditary |
| | cancer-predisposing syndrome; MUTYH-associated polyposis; MYH- |
| | associated polyposis; Neoplasm of stomach; Pilomatrixoma |
| MVK | Hyperimmunoglobulin D with periodic fever; Mevalonic aciduria; |
| | Disseminated superficial actinic type porokeratosis-3 |
| MYBPC3 | Asymmetric septal hypertrophy; Cardiomyopathy; Cardiovascular |
| | phenotype; Dyspnea; Familial dilated cardiomyopathy; Familial |
| | hypertrophic cardiomyopathy-1; Familial hypertrophic cardiomyopathy-4; |
| | Heart block; Hypertrophic cardiomyopathy; Inborn genetic diseases; Left |
| | ventricular hypertrophy; Left ventricular noncompaction; Left ventricular |
| | noncompaction-10; Long QT syndrome; MYBPC3-related disorders; |
| | Noncompaction cardiomyopathy; Primary dilated cardiomyopathy; Primary |
| | familial hypertrophic cardiomyopathy; Tachycardia; Ventricular |
| | extrasystoles |
| MYCN | Inborn genetic diseases |
| MYEF2, | Oculocutaneous albinism type VI |
| SLC24A5 | |
| MYF5 | Abnormality of the ribs; External ophthalmoplegia with rib and vertebral |
| | anomalies; External ophthalmoplegia; Scoliosis |
| MYH11, NDE1 | Familial aortopathy |
| MYH2, MYHAS | Proximal myopathy and ophthalmoplegia |
| MYH3 | Contractures, pterygia, and variable skeletal fusions syndrome 1A; |
| | Spondylocarpotarsal synostosis syndrome |
| MYH6 | Familial hypertrophic cardiomyopathy-1 |
| MYH7 | Hypertrophic cardiomyopathy; Primary dilated cardiomyopathy |
| MYH7, MHRT | Cardiomyopathy; Cardiovascular phenotype; Hypertrophic |
| | cardiomyopathy; MYH7-related disorders |
| MYL2, | Familial hypertrophic cardiomyopathy-10 |
| LOC114827850 | |
| MYLK | Visceral myopathy |
| MYO15A | Congenital sensorineural hearing impairment; Autosomal recessive |
| | deafness-3; Nonsyndromic hearing loss and deafness; Rare genetic deafness |
| MYO3A | Autosomal recessive deafness-30 |
| MYO5B | Congenital microvillous atrophy |
| MYO6 | Deafness; Autosomal dominant nonsyndromic hearing loss and deafness- |
| | 22; Rare genetic deafness |
| MYO7A | MYO7A-related disorders; Rare genetic deafness; Retinal dystrophy; |
| | Retinitis pigmentosa; Usher syndrome type 1B; Usher syndrome type 1; |
| | Autosomal dominant deafness-11; Autosomal recessive deafness-2 |
| MYOCD | Congenital megabladder; Prune belly syndrome |
| MYRF | Cardiac-urogenital syndrome |
| NADSYN1 | Vertebral, cardiac, renal and limb defects syndrome-3; Congenital NAD |
| | deficiency disorder |
| NAGLU | Charcot-Marie-Tooth disease axonal type 2V; MPS-III-B |
| | mucopolysaccharidosis; Sanfilippo syndrome |
| NALCN | Infantile hypotonia with psychomotor retardation and characteristic facies-1 |
| NBAS | Fever-associated acute infantile liver failure syndrome; Infantile liver |
| | failure syndrome-2 |
| NBN | Acute lymphoid leukemia; Aplastic anemia; Breast-ovarian cancer; Familial |
| | cancer of breast; Hereditary breast and ovarian cancer syndrome; Hereditary |
| | cancer-predisposing syndrome; Lissencephaly; Microcephaly with normal |
| | intelligence and immunodeficiency; Ovarian neoplasms |
| NCF1, | Autosomal recessive cytochrome b-positive chronic granulomatous disease |
| LOC106029312 | type 1; Chronic granulomatous disease due to deficiency of NCF-1; |
| | Autosomal recessive cytochrome b-positive chronic granulomatous disease |
| | type III |
| NCR1, NLRP7 | Recurrent hydatidiform mole-1 |
| NCSTN | Familial acne inversa-1 |
| NDE1 | Lissencephaly-4 |
| NDNF | Hypogonadotropic hypogonadism-25 with anosmia |
| NDUFA12 | Leigh syndrome |
| NDUFAF2 | Inborn genetic diseases; Leigh syndrome; Nuclear type 10 mitochondrial |
| | complex I deficiency; Nuclear type 1 mitochondrial complex I deficiency; |
| | NDUFAF2-related disorders |
| NDUFAF3 | Mitochondrial complex I deficiency |
| NDUFB11 | Linear skin defects with multiple congenital anomalies-3 |
| NDUFS4 | Leigh syndrome; Nuclear type 1 mitochondrial complex I deficiency |
| NDUFS6 | Nuclear type 9 mitochondrial complex I deficiency |
| NDUFV1 | Nuclear type 4 mitochondrial complex I deficiency; Nuclear type 1 |
| | mitochondrial complex I deficiency |
| NEB | Inborn genetic diseases; Nemaline myopathy; Nemaline myopathy-2; Non- |
| | immune hydrops fetalis |
| NEB, RIF1 | Nemaline myopathy; Nemaline myopathy-2 |
| NEBL | Hypertrophic cardiomyopathy; Long QT syndrome; Primary dilated |
| | cardiomyopathy; Primary familial hypertrophic cardiomyopathy; Sudden |
| | unexplained death |
| NEFL | Charcot-Marie-Tooth disease type 2E |
| NEK1 | Susceptibility to amyotrophic lateral sclerosis-24; Majewski type; Short rib- |
| | polydactyly syndrome; Short-rib thoracic dysplasia-3 with or without |
| | polydactyly |
| NEUROD1 | Maturity-onset diabetes of the young type 6 |
| NEXN | Dilated cardiomyopathy-1CC; Familial hypertrophic cardiomyopathy-20 |
| NF1 | Axillary freckling; Cafe-au-lait macules with pulmonary stenosis; Focal T2 |
| | hyperintense basal ganglia lesion; Ganglioglioma; Hereditary cancer- |
| | predisposing syndrome; Inborn genetic diseases; Juvenile myelomonocytic |
| | leukemia; Multiple cafe-au-lait spots; Familial spinal neurofibroma; |
| | Neurofibromas; Neurofibromatosis type 1; Neurofibromatosis-Noonan |
| | syndrome; Optic nerve glioma; Pilocytic astrocytoma; Tibial |
| | pseudoarthrosis |
| NF1, | Hereditary cancer-predisposing syndrome; Neurofibromatosis type 1 |
| LOC111811965 | |
| NF2 | Meningioma; Neurofibromatosis type 2 |
| NFIB | Acquired macrocephaly with impaired intellectual development; Intellectual |
| | disability; Macrocephaly; Macrocephalus |
| NFIX | Marshall-Smith syndrome |
| NGLY1 | Congenital disorder of deglycosylation; Intellectual disability; Neuromotor |
| | delay; Peripheral neuropathy |
| NHLRC1 | Lafora disease; Progressive myoclonic epilepsy-2 |
| NHLRC2 | Fibrosis, neurodegeneration, and cerebral angiomatosis |
| NHS | Nance-Horan syndrome |
| NIPAL4 | Autosomal recessive congenital ichthyosis-6 |
| NIPBL | Cornelia de Lange syndrome-1 |
| NKX2-5 | Abnormality of cardiovascular system morphology; Atrial septal defect-7 |
| | with or without atrioventricular conduction defects |
| NKX3-2 | Spondylo-megaepiphyseal-metaphyseal dysplasia |
| NKX6-2 | Autosomal recessive spastic ataxia-8 with hypomyelinating leukodystrophy |
| NLGN4X | Susceptibility to X-linked autism-2; Non-syndromic X-linked intellectual |
| | disability |
| NLRP7 | Recurrent hydatidiform mole-1 |
| NOTCH1 | Adams-Oliver syndrome-5; Aortic valve disorder; Congenital heart defect |
| NPC1 | Niemann-Pick disease type C; Niemann-Pick disease type C1 |
| NPHP1 | Nephronophthisis; Nephronophthisis-1 |
| NPHP3, NPHP3- | Meckel syndrome type 7 |
| ACAD11 | |
| NPHS1 | Finnish congenital nephrotic syndrome |
| NPHS2 | Idiopathic nephrotic syndrome; Idiopathic steroid-resistant nephrotic |
| | syndrome |
| NPHS2, | Idiopathic nephrotic syndrome; Nephrotic range proteinuria; Idiopathic |
| AXDND1 | steroid-resistant nephrotic syndrome |
| NPRL3, HBA- | Familial focal epilepsy with variable foci-3 |
| LCR | |
| NR0B1 | X-linked congenital adrenal hypoplasia |
| NR2E3 | Abnormality of color vision; Cone-rod dystrophy; Enhanced s-cone |
| | syndrome; Horizontal nystagmus; NR2E3-related disorders; Retinal |
| | dystrophy; Retinitis pigmentosa; Retinitis pigmentosa-37; Visual |
| | impairment |
| NR3C2 | Autosomal dominant pseudohypoaldosteronism type 1 |
| NSD1 | Beckwith-Wiedemann syndrome; Inborn genetic diseases; Sotos syndrome- |
| | 1 |
| NSD2 | 4p partial monosomy syndrome; Wolf-Hirschhorn like syndrome |
| NSMCE2 | Seckel syndrome-10 |
| NSMF | Hypogonadotropic hypogonadism-9 with or without anosmia |
| NSUN2 | Autosomal recessive mental retardation-5 |
| NT5E | Calcification of joints and arteries |
| NTHL1 | Familial adenomatous polyposis-3; Hereditary cancer-predisposing |
| | syndrome |
| NTRK1 | Hereditary insensitivity to pain with anhidrosis |
| OAT | Ornithine aminotransferase deficiency |
| OBSL1 | Three M syndrome-2 |
| OCA2 | Variation in skin/hair/eye pigmentation-1; Tyrosinase-positive |
| | oculocutaneous albinism |
| OCLN | Pseudo-TORCH syndrome-1 |
| OFD1 | Joubert syndrome; Orofaciodigital syndrome I; Simpson-Golabi-Behmel |
| | syndrome type 2 |
| OPA1 | Abortive cerebellar ataxia; Dominant hereditary optic atrophy; Inborn |
| | genetic diseases; Mitochondrial diseases; Retinal dystrophy |
| OPHN1 | Mental retardation X-linked with cerebellar hypoplasia and distinctive facial |
| | appearance |
| OPN1LW | Cone monochromatism |
| ORC6 | Meier-Gorlin syndrome-3 |
| OSGIN2, NBN | Hereditary cancer-predisposing syndrome; Microcephaly with normal |
| | intelligence and immunodeficiency |
| OTC | Abnormality of ornithine metabolism; Hyperammonemia; Ornithine |
| | carbamoyltransferase deficiency; Protein avoidance |
| OTOA | Autosomal recessive deafness-22; Rare genetic deafness |
| OTOF | Autosomal recessive deafness-9; Rare genetic deafness |
| OTOG | Autosomal recessive deafness-18B; Intellectual disability; Rare genetic |
| | deafness; Seizures |
| OTOGL | Rare genetic deafness |
| OTUD6B | Dysmorphic features; Epilepsy; Intellectual developmental disorder with |
| | dysmorphic facies, seizures, and distal limb anomalies; Intellectual |
| | disability |
| OTX2 | Syndromic microphthalmia type 5 |
| P2RY12, | Platelet-type bleeding disorder-8 |
| MED12L | |
| P3H1 | Osteogenesis imperfecta type 8 |
| P3H2 | High myopia with cataract and vitreoretinal degeneration |
| P4HA2 | Autosomal dominant myopia-25 |
| PAFAH1B1 | Inborn genetic diseases; Lissencephaly due to LIS1 mutation |
| PAH | Phenylketonuria |
| PALB2 | Basal cell carcinoma; Breast cancer; Cancer of the pancreas; Familial |
| | cancer of breast; Complementation group N Fanconi anemia; Generalized |
| | hypopigmentation; Hereditary breast and ovarian cancer syndrome; |
| | Hereditary cancer; Hereditary cancer-predisposing syndrome; Neoplasm of |
| | the breast; Ovarian neoplasms; PALB2-related disorders; Susceptibility to |
| | pancreatic cancer-3; Pre-B-cell acute lymphoblastic leukemia; |
| | Tracheoesophageal fistula; Tumor susceptibility linked to germline BAP1 |
| | mutations |
| PANK2 | Pigmentary pallidal degeneration |
| PAPSS2 | Pakistani type spondyloepimetaphyseal dysplasia |
| PARN | Autosomal recessive dyskeratosis congenita-6 |
| PATL2 | Oocyte maturation defect-4 |
| PAX2 | Focal segmental glomerulosclerosis-7; Renal coloboma syndrome |
| PAX3 | Rare genetic deafness; Waardenburg syndrome; Waardenburg syndrome |
| | type 1 |
| PAX6 | Aniridia-1; Autosomal dominant keratitis |
| PAX9 | Selective tooth agenesis-3 |
| PC | Pyruvate carboxylase deficiency |
| PCCA | Propionic acidemia |
| PCCB | Propionic acidemia |
| PCDH15 | Deafness; Autosomal dominant nonsyndromic deafness-23; Rare genetic |
| | deafness; Retinal dystrophy; Digenic Usher syndrome type ID/F; Usher |
| | syndrome type 1G; Usher syndrome type 1; Usher syndrome type 1D; |
| | Usher syndrome type 1F |
| PCDH19 | Absence seizures; Delayed speech and language development; Early |
| | infantile epileptic encephalopathy-9; Frontal cortical atrophy; Generalized |
| | seizures; Generalized tonic-clonic seizures; Global developmental delay; |
| | Hand tremor; Long palpebral fissure; Prominent fingertip pads; Strabismus; |
| | Temporal cortical atrophy |
| PCLO | Pontocerebellar hypoplasia type 3 |
| PCNT | Microcephalic osteodysplastic primordial dwarfism type II |
| PCSK1, | Proprotein convertase 1/3 deficiency |
| LOC101929710 | |
| PCSK9 | Familial hypercholesterolemia; Familial hypercholesterolemia-1; Low |
| | density lipoprotein cholesterol level quantitative trait locus 1 |
| PCYT1A | Spondylometaphyseal dysplasia-cone-rod dystrophy syndrome |
| PDE11A | Primary pigmented nodular adrenocortical disease-2 |
| PDE6B | Retinal dystrophy; Retinitis pigmentosa; Retinitis pigmentosa-40 |
| PDE6C | Achromatopsia-5 |
| PDE8B | Autosomal dominant striatal degeneration-1 |
| PDHA1 | Inborn genetic diseases; Pyruvate dehydrogenase E1-alpha deficiency |
| PDX1 | Permanent neonatal diabetes mellitus-1; Maturity-onset diabetes of the |
| | young type 4; Pancreatic agenesis-1 |
| PDZD7 | Autosomal recessive deafness-57; Rare genetic deafness; Usher syndrome |
| | type 2A |
| PEPD | Prolidase deficiency |
| PEX1 | Deafness enamel hypoplasia nail defects; Zellwegar peroxisome biogenesis |
| | disorder-1A; Peroxisome biogenesis disorder-1B; Peroxisome biogenesis |
| | disorders; Retinal dystrophy; Zellweger syndrome spectrum |
| PEX1, GATAD1 | Deafness/enamel hypoplasia/nail defects; Zellweger peroxisome biogenesis |
| | disorder 1A; Peroxisome biogenesis disorder 1B; Peroxisome biogenesis |
| | disorders; Zellweger syndrome spectrum |
| PEX10 | Complementation group 7 peroxisome biogenesis disorder; Peroxisome |
| | biogenesis disorder-6A; Peroxisome biogenesis disorder-6B; Peroxisome |
| | biogenesis disorders; Zellweger syndrome spectrum |
| PEX10, PLCH2 | Peroxisome biogenesis disorder-6B |
| PEX12 | Infantile Refsum's disease; Peroxisome biogenesis disorder-3A; |
| | Peroxisome biogenesis disorders; Zellweger syndrome spectrum |
| PEX2 | Peroxisome biogenesis disorder-5B; Zellweger peroxisome biogenesis |
| | disorder-5A; Peroxisome biogenesis disorders; Zellweger syndrome |
| | spectrum |
| PEX26 | Peroxisome biogenesis disorder-7A; Peroxisome biogenesis disorder-7B; |
| | Peroxisome biogenesis disorders; Zellweger syndrome spectrum |
| PEX6 | Heimler syndrome-2; Peroxisome biogenesis disorder-4B; Zellweger |
| | peroxisome biogenesis disorder-4A; Peroxisome biogenesis disorders; |
| | Retinal dystrophy; Zellweger syndrome spectrum |
| PEX7 | PEX7-related disorders; Peroxisome biogenesis disorder-9B; Phytanic acid |
| | storage disease; Rhizomelic chondrodysplasia punctata type 1 |
| PGAM2, DBNL | Glycogen storage disease type X |
| PGAP1 | Autosomal recessive mental retardation-42 |
| PGAP3 | Hyperphosphatasia with mental retardation syndrome-4 |
| PGM3, DOP1A | Immunodeficiency-23 |
| PHEX | Familial X-linked hypophosphatemic vitamin D refractory rickets |
| PHEX, | Familial X-linked hypophosphatemic vitamin D refractory rickets |
| PTCHD1-AS | |
| PHF3, EYS | Retinal dystrophy; Retinitis pigmentosa-25 |
| PHF6 | Borjeson-Forssman-Lehmann syndrome |
| PHGDH | Phosphoglycerate dehydrogenase deficiency |
| PHIP | Developmental delay, intellectual disability, obesity, and dysmorphic |
| | features |
| PHYH | Phytanic acid storage disease; Adult refsum disease-1 |
| PI4KA | Perisylvian polymicrogyria with cerebellar hypoplasia and arthrogryposis |
| PIGA | Paroxysmal nocturnal hemoglobinuria-1 |
| PIGN | Multiple congenital anomalies-hypotonia-seizures syndrome-1 |
| PIGO | Hyperphosphatasia with mental retardation syndrome-2; |
| | Hyperphosphatasia-intellectual disability syndrome |
| PIGT | Multiple congenital anomalies-hypotonia-seizures syndrome-3; PIGT- |
| | related disorder |
| PIK3R1 | SHORT syndrome |
| PINK1 | Autosomal recessive early-onset Parkinson disease-6 |
| PIRC66, | Aromatase deficiency |
| MIR 4713HG, | |
| CYP19A1 | |
| PITX3 | Anterior segment mesenchymal dysgenesis; Cataract-11 |
| PJVK | Autosomal recessive deafness-59; Rare genetic deafness |
| PKD1 | Autosomal recessive polycystic kidney disease; Adult type polycystic |
| | kidney disease |
| PKD1, | Adult type polycystic kidney disease |
| LOC105371049 | |
| PKHD1 | Autosomal recessive polycystic kidney disease; Polycystic kidney |
| | dysplasia; Polycystic liver disease |
| PKP1 | Epidermolysis bullosa simplex due to plakophilin deficiency |
| PKP2 | Arrhythmogenic right ventricular cardiomyopathy; Arrhythmogenic right |
| | ventricular dysplasia/cardiomyopathy; Arrhythmogenic ventricular |
| | cardiomyopathy type 9; Cardiac arrhythmia; Cardiomyopathy; |
| | Cardiovascular phenotype; Sudden unexplained death |
| PLA2G5 | Familial benign fleck retina |
| PLA2G6 | Infantile neuroaxonal dystrophy; Iron accumulation in brain; |
| | Neurodegeneration with brain iron accumulation-2B; PLA2G6-associated |
| | neurodegeneration |
| PLCB1 | Early infantile epileptic encephalopathy-12 |
| PLCB4 | Auriculocondylar syndrome-2 |
| PLCD1 | Leukonychia totalis |
| PLD1 | Developmental cardiac valvular defect |
| PLD3, PRX | Charcot-Marie-Tooth disease; Spinocerebellar ataxia-46 |
| PLEC | Epidermolysis bullosa simplex with muscular dystrophy |
| PLN, CEP85L | Cardiac arrest; Cardiomyopathy; Cardiovascular phenotype; Dilated |
| | cardiomyopathy-1P; Familial hypertrophic cardiomyopathy-18; |
| | Hypertrophic cardiomyopathy; Primary dilated cardiomyopathy; Sudden |
| | cardiac death |
| PLOD1 | Cardiovascular phenotype; Hydroxylysine-deficient Ehlers-Danlos |
| | syndrome |
| PLOD2 | Bruck syndrome-2 |
| PLP1, RAB9B | Hereditary spastic paraplegia-2 |
| PLS3 | Bone mineral density quantitative trait locus 18 |
| PMFBP1 | Spermatogenic failure-31 |
| PMM2 | Congenital disorder of glycosylation type IA |
| PMP22 | Charcot-Marie-Tooth disease |
| PMS2 | Acute lymphoid leukemia; Burkitt lymphoma; Colorectal cancer; |
| | Glioblastoma; Hereditary cancer; Hereditary cancer-predisposing |
| | syndrome; Hereditary nonpolyposis colon cancer; Hereditary nonpolyposis |
| | colorectal cancer type 4; Lymphoma; Lynch syndrome; Lynch syndrome-I; |
| | Pulmonary arterial hypertension; Pulmonary insufficiency; Respiratory |
| | insufficiency; Tumor susceptibility linked to germline BAP1 mutations; |
| | Turcot syndrome |
| PNKD, CATIP- | Paroxysmal nonkinesigenic dyskinesia-1 |
| AS2 | |
| PNKP | Ataxia-oculomotor apraxia-4; Early infantile epileptic encephalopathy-10; |
| | Early infantile epileptic encephalopathy-12; History of neurodevelopmental |
| | disorder |
| PNPLA2 | Neutral lipid storage myopathy |
| PNPLA6 | Hereditary spastic paraplegia-39; Laurence-Moon syndrome; PNPLA6- |
| | related disorders; Trichomegaly-retina pigmentary degeneration-dwarfism |
| | syndrome |
| PNPLA8 | Mitochondrial myopathy-lactic acidosis-deafness syndrome |
| PNPO | Pyridoxal phosphate-responsive seizures |
| POC5 | Retinitis pigmentosa; Syndromic retinitis pigmentosa |
| POGLUT1 | Dowling-degos disease-4 |
| POGZ | Global developmental delay; Speech apraxia; White-sutton syndrome; |
| | Dysmorphy; Intellectual deficiency |
| POLA1 | Van Esch-O'Driscoll syndrome; Van Esch type X-linked intellectual |
| | disability |
| POLD1 | Colorectal cancer-10; Hereditary cancer-predisposing syndrome; |
| | Mandibular hypoplasia, deafness, progeroid features and lipodystrophy |
| | syndrome |
| POLE | Susceptibility to colorectal cancer-12; Hereditary cancer-predisposing |
| | syndrome; Intrauterine growth retardation, metaphyseal dysplasia, adrenal |
| | hypoplasia congenita, genital anomalies, and immunodeficiency |
| POLG | Generalized epilepsy; Global developmental delay; Obesity; Progressive |
| | sclerosing poliodystrophy; Seizures |
| POLH | Variant type xeroderma pigmentosum |
| POLR1A | Cincinnati type acrofacial dysostosis |
| POLR1C | Treacher Collins syndrome-3 |
| POLR1D | Treacher Collins syndrome-2 |
| POLR2F, | Rare genetic deafness; Waardenburg syndrome type 4C |
| SOX10 | |
| POLR3A | Hypomyelinating leukodystrophy-7; Neonatal pseudo-hydrocephalic |
| | progeroid syndrome |
| POLR3B | Cerebellar hypoplasia with endosteal sclerosis; Hypogonadotropic |
| | hypogonadism-7 with or without anosmia; Hypomyelinating |
| | leukodystrophy-7; Hypomyelinating leukodystrophy-8 with or without |
| | oligodontia and/or hypogonadotropic hypogonadism |
| POMK | Limb-girdle muscular dystrophy-dystroglycanopathy type C-12 |
| POMT1 | Congenital muscular dystrophy-dystroglycanopathy with mental retardation |
| | type B1; Limb-girdle muscular dystrophy-dystroglycanopathy type C1; |
| | Congenital muscular dystrophy-dystroglycanopathy type A1 with brain and |
| | eye anomalies; POMT1-related disorders; Walker-Warburg congenital |
| | muscular dystrophy |
| POMT2 | Congenital muscular dystrophy-dystroglycanopathy with brain and eye |
| | anomalies type A2; Limb-girdle muscular dystrophy-dystroglycanopathy |
| | type C2 |
| POP1 | Anauxetic dysplasia-2 |
| POR | Antley-Bixler syndrome with genital anomalies and disordered |
| | steroidogenesis; Disordered steroidogenesis due to cytochrome p450 |
| | oxidoreductase deficiency |
| PORCN | Focal dermal hypoplasia |
| POT1 | Hereditary cancer-predisposing syndrome; Susceptibility to cutaneous |
| | malignant melanoma-10 |
| POU3F4 | X-linked deafness-2; Rare genetic deafness |
| POU4F3 | Rare genetic deafness |
| PPARG | Type II diabetes mellitus; Digenic noninsulin-dependent diabetes mellitus |
| | with acanthosis nigricans and hypertension |
| PPIB | Osteogenesis imperfecta type 9 |
| PPOX | Variegate porphyria |
| PPP1R12A | Genitourinary and/or brain malformation syndrome |
| PPT1 | History of neurodevelopmental disorder; Neuronal ceroid-lipofuscinosis; |
| | Recessive neuronal ceroid lipofuscinosis; Neuronal ceroid lipofuscinosis-1 |
| PQBP1 | Delayed speech and language development; Hyperactivity; Inborn genetic |
| | diseases; Intellectual disability; Microcephaly; Renpenning syndrome-1 |
| PRDM16 | Left ventricular noncompaction-8 |
| PRDM5 | Brittle cornea syndrome-2 |
| PRDX1, | Digenic cblC type methylmalonic aciduria and homocystinuria |
| MMACHC | |
| PRF1 | Familial hemophagocytic lymphohistiocytosis; Familial hemophagocytic |
| | lymphohistiocytosis-2 |
| PRKAR1A | Carney complex type 1 |
| PRKAR1A, | Amelogenesis imperfecta type 1G |
| FAM20A | |
| PRKAR1B, | Primary ciliary dyskinesia-18 |
| DNAAF5 | |
| PRKCSH | Polycystic liver disease-1 |
| PRKN | Parkinson disease-2 |
| PRMT7 | Short stature, brachydactyly, intellectual developmental disability, and |
| | seizures |
| PROK2 | Hypogonadotropic hypogonadism-4 with or without anosmia |
| PROKR2 | Inborn genetic diseases; Kallmann syndrome-3 |
| PROM1 | Cone-rod dystrophy-12; PROM1-related disorders; Retinal dystrophy; |
| | Retinitis pigmentosa; Retinitis pigmentosa-41 |
| PROP1 | Combined pituitary hormone deficiency-2 |
| PRPH2 | Adult-onset vitelliform macular dystrophy; Retinal dystrophy; Retinitis |
| | pigmentosa-7; Retinitis punctata albescens |
| PRRT2 | Episodic kinesigenic dyskinesia-1; History of neurodevelopmental disorder; |
| | Infantile convulsions and choreoathetosis; Paroxysmal kinesigenic |
| | dyskinesia; Paroxysmal nonkinesigenic dyskinesia-1; Benign familial |
| | infantile seizures-2 |
| PRSS12 | Autosomal recessive mental retardation-1 |
| PRSS56 | Isolated microphthalmia-6 |
| PRX | Demyelinating Charcot-Marie-Tooth disease type 4F |
| PSAP | Combined saposin deficiency |
| PSEN1 | Familial acne inversa-3 |
| PSENEN | Familial acne inversa-2 |
| PTCH1 | Gorlin syndrome; Hereditary cancer-predisposing syndrome |
| PTCH2 | Gorlin syndrome; Medulloblastoma |
| PTEN | Cowden syndrome; Cowden syndrome-1; Glioblastoma; Glioma |
| | susceptibility-2; Hemangioma; Hereditary cancer-predisposing syndrome; |
| | Inborn genetic diseases; Macrocephaly/autism syndrome; Malignant tumor |
| | of prostate; Familial meningioma; Neoplasm of brain; Neoplasm of the |
| | breast; Neoplasm of the large intestine; Non-small cell lung cancer; Ovarian |
| | neoplasms; PTEN hamartoma tumor syndrome; PTEN-related disorder; |
| | Proteus-like syndrome; VACTERL association with hydrocephalus |
| PTH1R | Blomstrand type chondrodysplasia |
| PTPN11 | Metachondromatosis |
| PTPRF | Aplasia or hypoplasia of breasts and/or nipples-2 |
| PTPRO | Nephrotic syndrome type 6 |
| PTS | BH4-deficient hyperphenylalaninemia-A; BH4-deficient |
| | hyperphenylalaninemia due to partial PTS deficiency |
| PUF60 | Verheij syndrome |
| PURA | Apnea; Generalized hypotonia; Autosomal dominant intellectual disability- |
| | 31; Limb dystonia; Mental retardation; PURA Syndrome; PURA-related |
| | severe neonatal hypotonia-seizures-encephalopathy syndrome due to a point |
| | mutation |
| PUS7 | Intellectual developmental disorder with abnormal behavior, microcephaly, |
| | and short stature |
| PXDN | Anterior segment dysgenesis-7 |
| PYCR1 | Autosomal recessive cutis laxa type 2B |
| PYGL | Glycogen storage disease type VI |
| PYGM | Glycogen storage disease type V |
| RAB23 | Carpenter syndrome; Carpenter syndrome-1 |
| RAB27A | Griscelli syndrome; Griscelli syndrome type 2 |
| RAB33B | Smith-McCort dysplasia-2 |
| RAB3GAP1 | Warburg micro syndrome-1 |
| RABL3 | Susceptibility to pancreatic cancer-5 |
| RAD50 | Hereditary cancer-predisposing syndrome; Nijmegen breakage syndrome- |
| | like disorder |
| RAD51C | Familial breast-ovarian cancer-3; Complementation group O Fanconi |
| | anemia; Hereditary breast and ovarian cancer syndrome; Hereditary cancer- |
| | predisposing syndrome; Ovarian neoplasms; RAD51C-related disorders |
| RAD51D, | Familial breast-ovarian cancer-4; Hereditary breast and ovarian cancer |
| RAD51L3-RFFL | syndrome; Hereditary cancer-predisposing syndrome; Ovarian neoplasms |
| RAD51L3- | Familial breast-ovarian cancer-4; Hereditary cancer-predisposing syndrome |
| RFFL, RAD51D | |
| RAI1 | Smith-Magenis syndrome |
| RAPSN | Congenital myasthenic syndrome-11 associated with acetylcholine receptor |
| | deficiency |
| RARS1 | Hypomyelinating leukodystrophy-9 |
| RASA1 | Capillary malformation-arteriovenous malformation; Capillary |
| | malformation-arteriovenous malformation-1 |
| RB1 | Hereditary cancer-predisposing syndrome; Neoplasm; Osteosarcoma; |
| | Trilateral retinoblastoma; Small cell lung cancer; Urinary bladder cancer |
| RBBP8 | Microcephaly with mental retardation and digital anomalies |
| RBM20 | Cardiovascular phenotype; Dilated cardiomyopathy-1DD; Primary dilated |
| | cardiomyopathy |
| RBP3 | Retinitis pigmentosa-66 |
| RD3 | Leber congenital amaurosis-12 |
| RDH12 | Retinitis pigmentosa-53 |
| RDH5, | Fundus albipunctatus; Pigmentary retinal dystrophy |
| BLOC1S1- | |
| RDH5 | |
| RECQL | Hereditary cancer-predisposing syndrome |
| RECQL, | Hereditary cancer-predisposing syndrome |
| PYROXD1 | |
| RECQL4 | B lymphoblastic leukemia with t(12; 21)(p13; q22); Baller-Gerold syndrome; |
| | High grade surface osteosarcoma; Rapadilino syndrome; Rothmund- |
| | Thomson syndrome; Rothmund-Thomson syndrome type 2 |
| REEP6 | Retinitis pigmentosa-77 |
| RELT | Amelogenesis imperfecta type IIIC |
| REN | Familial hyperproreninemia |
| RET | Hirschsprung disease-1; Sensorineural hearing loss |
| RFX5 | Complementation group C bare lymphocyte syndrome type II |
| RFXANK | Complementation group B bare lymphocyte syndrome type II |
| RFXAP | Complementation group D bare lymphocyte syndrome type II; Bare |
| | lymphocyte syndrome-2 |
| RHAG | Regulatory type Rh-null disease |
| RHCE | Amorph type Rh-null disease |
| RHO | Autosomal dominant retinitis pigmentosa |
| RIF1, NEB | Nemaline myopathy; Nemaline myopathy-2 |
| RIN2 | Macrocephaly, alopecia, cutis laxa, and scoliosis |
| RIPK1 | Immunodeficiency-57 with autoinflammation |
| RIPK4 | Bartsocas-Papas syndrome |
| RNASEH2A | Aicardi Goutieres syndrome-4 |
| RNASEH2B | Aicardi Goutieres syndrome-2 |
| RNF113A | Nonphotosensitive trichothiodystrophy-5 |
| RNF216 | Gordon Holmes syndrome |
| ROBO3 | Familial horizontal gaze palsy with progressive scoliosis-1 |
| RORA, RORA- | Intellectual developmental disorder with or without epilepsy or cerebellar |
| AS1 | ataxia |
| RP1 | Retinal dystrophy; Retinitis pigmentosa; Retinitis pigmentosa-1 |
| RP1L1 | Retinitis pigmentosa-88 |
| RPE65 | Leber congenital amaurosis-2; Retinitis pigmentosa-87 with choroidal |
| | involvement; RPE65-related disorders; Retinal dystrophy; Retinitis |
| | pigmentosa-20 |
| RPGR | Inborn genetic diseases; Retinal dystrophy; X-linked retinitis pigmentosa |
| | and sinorespiratory infections with deafness; Retinitis pigmentosa-15 |
| RPGRIP1 | Leber congenital amaurosis-6 |
| RPGRIP1L | Joubert syndrome; Joubert syndrome-7 |
| RPL36A- | Fabry disease |
| HNRNPH2, | |
| GLA | |
| RPL5, DIPK1A | Diamond-Blackfan anemia; Diamond-Blackfan anemia-1 |
| RPS10, RPS10- | Diamond-Blackfan anemia-9 |
| NUDT3 | |
| RPS27 | Diamond-Blackfan anemia-17 |
| RPS6KA3 | Coffin-Lowry syndrome; X-linked mental retardation-19 |
| RSPH1 | Kartagener syndrome; Primary ciliary dyskinesia; Primary ciliary |
| | dyskinesia-24 |
| RSPH4A | Primary ciliary dyskinesia-11; Kartagener syndrome; Primary ciliary |
| | dyskinesia |
| RSPO2 | Tetraamelia syndrome-2 |
| RTEL1, RTEL1- | Autosomal recessive dyskeratosis congenita-5; Chronic form idiopathic |
| TNFRSF6B | fibrosing alveolitis; Telomere-related pulmonary fibrosis and/or bone |
| | marrow failure-3; Autosomal dominant dyskeratosis congenita-4 |
| RTN2 | Hereditary spastic paraplegia-12 |
| RTTN | Congenital microcephaly; Microcephaly, short stature, and polymicrogyria |
| | with or without seizures |
| RUNX1 | Acute myeloid leukemia; Familial platelet disorder with associated myeloid |
| | malignancy |
| RYR1 | Central core myopathy; Susceptibility to malignant hyperthermia-1; |
| | Minicore myopathy; Multi-minicore disease and atypical periodic paralysis; |
| | Neuromuscular disease; RYR1-related disorders |
| SACS | Autosomal recessive spastic ataxia; Charlevoix-Saguenay spastic ataxia; |
| | Spastic paraplegia |
| SAG | Oguchi's disease; Retinitis pigmentosa-47; SAG-related disorders |
| SALL1 | Townes syndrome |
| SAMD9L | Ataxia-pancytopenia syndrome |
| SAMHD1 | Aicardi Goutieres syndrome-5 |
| SASH1 | Dyschromatosis universalis hereditarian-1 |
| SATB2 | SATB2-related disorder |
| SBDS | Inborn genetic diseases; Shwachman-Diamond syndrome-1 |
| SBF1 | Charcot-Marie-Tooth disease type 4 |
| SCAPER | Attention deficit hyperactivity disorder; Intellectual developmental disorder |
| | and retinitis pigmentosa; Intellectual disability; Rod-cone dystrophy |
| SCARB2 | Progressive myoclonic epilepsy-4 with or without renal failure |
| SCARF2 | Van den Ende-Gupta syndrome |
| SCN1A | Autosomal dominant epilepsy; Early infantile epileptic encephalopathy; |
| | Familial hemiplegic migraine type 3; Generalized epilepsy with febrile |
| | seizures plus type 2; History of neurodevelopmental disorder; Severe |
| | myoclonic epilepsy in infancy; Dravet syndrome |
| SCN1A, | Autosomal dominant epilepsy; Early infantile epileptic encephalopathy; |
| LOC102724058 | Epileptic encephalopathy; Generalized epilepsy with febrile seizures plus |
| | type 2; Seizures; Severe myoclonic epilepsy in infancy |
| SCN2A | SCN2A-related disorder |
| SCN5A | Brugada syndrome; Brugada syndrome with shorter-than-normal QT |
| | interval; Brugada syndrome-1; Cardiovascular phenotype; Dilated |
| | cardiomyopathy-1E; Nonprogressive heart block; Long QT syndrome-1 |
| SCN5A, | Brugada syndrome; Brugada syndrome with shorter-than-normal QT |
| LOC110121269 | interval |
| SCN9A, | Generalized epilepsy with febrile seizures plus type 7; Hereditary sensory |
| SCN1A-AS1 | and autonomic neuropathy type IIA; Autosomal recessive congenital |
| | indifference to pain |
| SCNN1A | Autosomal recessive pseudohypoaldosteronism type 1; Idiopathic |
| | bronchiectasis |
| SCNN1B | Liddle syndrome-1 |
| SCNN1G | Autosomal recessive pseudohypoaldosteronism type 1; Liddle syndrome-2 |
| SCO1 | Mitochondrial complex IV deficiency |
| SCP2 | Leukoencephalopathy with dystonia and motor neuropathy |
| SDCCAG8 | Bardet-Biedl syndrome; Bardet-Biedl syndrome-16; Senior-Loken |
| | syndrome-7 |
| SDHA | Carney triad; Dilated cardiomyopathy-1GG; Hereditary cancer-predisposing |
| | syndrome; Leigh syndrome; Mitochondrial complex II deficiency; |
| | Paragangliomas-5; Pilocytic astrocytoma |
| SDHAF2 | Hereditary paraganglioma-pheochromocytoma syndromes |
| SDHB | Carney-Stratakis syndrome; Gastrointestinal stromal tumor; Hereditary |
| | paraganglioma-pheochromocytoma syndromes; Hereditary cancer- |
| | predisposing syndrome; Paragangliomas-4; Pheochromocytoma |
| SDHC | Gastrointestinal stromal tumor; Hereditary paraganglioma- |
| | pheochromocytoma syndromes; Hereditary cancer-predisposing syndrome; |
| | Paragangliomas-3 |
| SDHD | Carney-Stratakis syndrome; Cowden syndrome-3; Hereditary |
| | paraganglioma-pheochromocytoma syndromes; Hereditary cancer- |
| | predisposing syndrome; Paragangliomas-1; Paragangliomas-1 with |
| | sensorineural hearing loss; Pheochromocytoma |
| SDR9C7 | Autosomal recessive congenital ichthyosis-13 |
| SEC23B | Congenital dyserythropoietic anemia |
| SEC24D | Cole-Carpenter syndrome-2 |
| SECISBP2 | Anormal thyroid hormone metabolism |
| SELENBP1 | Extraoral halitosis due to methanethiol oxidase deficiency; Extraoral |
| | halitosis |
| SELENON | Eichsfeld type congenital muscular dystrophy |
| SEMA3A | Hypogonadotropic hypogonadism-16 with or without anosmia |
| SEPSECS | Pontocerebellar hypoplasia type 2D |
| SEPTIN12 | Spermatogenic failure-10 |
| SERAC1 | 3-methylglutaconic aciduria with deafness, encephalopathy, and Leigh-like |
| | syndrome; Mitochondrial oxidative phosphorylation disorder |
| SERPINA6 | Corticosteroid-binding globulin deficiency |
| SERPINA7 | Thyroxine-binding globulin quantitative trait locus |
| SERPINB6 | Rare genetic deafness |
| SERPINB7 | Nagashima type palmoplantar keratoderma |
| SERPINC1 | Antithrombin III deficiency |
| SERPINF1 | Osteogenesis imperfecta type VI |
| SERPING1 | Hereditary angioedema type 1 |
| SERPINH1 | Osteogenesis imperfecta type 10 |
| SETBP1 | SETBP1-related disorder |
| SETD5 | Inborn genetic diseases; Autosomal dominant mental retardation-23 |
| SF3B4 | Hereditary hearing loss and deafness; Inborn genetic diseases; Nager |
| | syndrome |
| SFRP4 | Pyle metaphyseal dysplasia |
| SFTPA1 | Respiratory distress associated with prematurity |
| SFTPB | Pulmonary surfactant metabolism dysfunction-1 |
| SGCA | Autosomal recessive limb-girdle muscular dystrophy type 2D |
| SGCD | Neuromuscular disease |
| SGCE, CASD1 | Myoclonic dystonia |
| SGCG | North African type severe autosomal recessive muscular dystrophy of |
| | childhood |
| SGSH | Developmental regression; Diarrhea; Gastrointestinal dysmotility; Global |
| | developmental delay; MPS-III-A mucopolysaccharidosis; Nystagmus; |
| | Retinal dystrophy; Sanfilippo syndrome; Severe visual impairment |
| SH2D1A | X-linked lymphoproliferative syndrome-1; X-Linked Lymphoproliferative |
| | Syndrome |
| SH3PXD2B | Frank-Ter Haar syndrome |
| SH3TC2 | Charcot-Marie-Tooth disease type 4C; Charcot-Marie-Tooth disease type 4; |
| | Inborn genetic diseases; Mild mononeuropathy of the median nerve; |
| | SH3TC2-related disorders |
| SHANK3 | 22q13.3 deletion syndrome; Autism spectrum disorder; History of |
| | neurodevelopmental disorder; Inborn genetic diseases; SHANK3-related |
| | disorder |
| SHOX | Leri-Weill dyschondrosteosis |
| SI | Sucrase-isomaltase deficiency |
| SIX6 | Colobomatous optic disc-macular atrophy-chorioretinopathy syndrome |
| SKIV2L | Trichohepatoenteric syndrome-2 |
| SLC10A7 | Amelogenesis imperfecta, short stature, and skeletal dysplasia with scoliosis |
| SLC12A1 | Antenatal Bartter syndrome type 1 |
| SLC12A3 | Familial hypokalemia-hypomagnesemia |
| SLC12A6 | Agenesis of the corpus callosum with peripheral neuropathy; Charcot- |
| | Marie-Tooth disease |
| SLC17A5 | Salla disease; Severe infantile type sialic acid storage disease |
| SLC19A1, | Knobloch syndrome-1 |
| COL18A1 | |
| SLC19A2 | Thiamine-responsive megaloblastic anemia with diabetes mellitus and |
| | sensorineural deafness |
| SLC19A3 | Biotin-responsive basal ganglia disease |
| SLC22A5 | Renal carnitine transport defect |
| SLC25A20 | Carnitine acylcarnitine translocase deficiency |
| SLC26A2 | 3MC syndrome-2; Achondrogenesis type IB; Atelosteogenesis type II; |
| | Diastrophic dysplasia; Multiple epiphyseal dysplasia type 4; |
| | Osteochondrodysplasia; SLC26A2-related disorders |
| SLC26A3 | Congenital chloride type secretory diarrhea |
| SLC26A4 | Enlarged vestibular aqueduct; Pendred syndrome; Rare genetic deafness |
| SLC2A10 | Arterial tortuosity syndrome; Cardiovascular phenotype |
| SLC2A2 | Fanconi-Bickel syndrome |
| SLC30A8 | Diabetes mellitus type 2 |
| SLC33A1 | Autosomal dominant spastic paraplegia; Spastic paraplegia-42 |
| SLC34A3 | Autosomal recessive hypophosphatemic bone disease |
| SLC35A2 | Congenital disorder of glycosylation type IIM |
| SLC35D1 | Schneckenbecken dysplasia |
| SLC37A4 | Glucose-6-phosphate transport defect; Glycogen storage disease; Inborn |
| | genetic diseases; Phosphate transport defect |
| SLC38A8 | Foveal hypoplasia-2 with optic nerve misrouting and anterior segment |
| | dysgenesis |
| SLC39A4 | Hereditary acrodermatitis enteropathica |
| SLC45A2 | Oculocutaneous albinism type 4 |
| SLC4A1 | Autosomal dominant distal renal tubular acidosis |
| SLC4A11 | Corneal endothelial dystrophy; Fuchs endothelial corneal dystrophy-4 |
| SLC52A3 | Brown-Vialetto-Van Laere syndrome-1 |
| SLC6A1 | Myoclonic-atonic epilepsy; SLC6A1-related disorder |
| SLC9A3 | Congenital secretory sodium diarrhea-8 |
| SLC9A3, | Congenital secretory sodium diarrhea-8 |
| SLC9A3-AS1 | |
| SLC9A6 | Gastrostomy tube feeding in infancy; Global developmental delay; |
| | Recurrent respiratory infections; Scoliosis; Seizures; Sleep disturbance |
| SLCO2A1 | Autosomal recessive primary hypertrophic osteoarthropathy-2 |
| SLITRK1 | Tourette Syndrome; Trichotillomania |
| SLURP1 | Acroerythrokeratoderma |
| SMAD3 | Familial thoracic aortic aneurysm and aortic dissection |
| SMAD4 | Carcinoma of pancreas; Hereditary cancer-predisposing syndrome; Juvenile |
| | polyposis syndrome; Juvenile polyposis/hereditary hemorrhagic |
| | telangiectasia syndrome; Myhre syndrome |
| SMAD6 | Aortic valve disease-2; Aortic valve disorder; Susceptibility to |
| | craniosynostosis-7 |
| SMARCA4 | Neuroblastoma |
| SMARCAL1 | Schimke immuno-osseous dysplasia |
| SMARCB1 | Atypical teratoid tumor |
| SMARCE1 | Familial meningioma |
| SMC1A | Congenital muscular hypertrophy-cerebral syndrome; Early infantile |
| | epileptic encephalopathy-85 with or without midline brain defects |
| SMN1 | Werdnig-Hoffmann disease |
| SMPD1 | Niemann-Pick disease type A; Niemann-Pick disease type B; |
| | Sphingomyelin/cholesterol lipidosis |
| SNAP29 | CEDNIK syndrome; Hypomyelinating leukodystrophy-2 |
| SNRPB | Cerebro-costo-mandibular syndrome |
| SOHLH1 | Nonsyndromic hypergonadotropic hypogonadism; Ovarian dysgenesis-5 |
| SON | Inborn genetic diseases; ZTTK syndrome |
| SOS1 | Gingival fibromatosis-1 |
| SOX2, SOX2- | Anophthalmia/microphthalmia-esophageal atresia syndrome |
| OT | |
| SOX9 | Campomelic dysplasia with autosomal sex reversal; Camptomelic dysplasia |
| SOX9, | Campomelic dysplasia with autosomal sex reversal |
| LOC108021846 | |
| SP110, SP140 | Hepatic veno-occlusive disease-immunodeficiency syndrome |
| SP7 | Osteogenesis imperfecta type 12 |
| SPART | Troyer syndrome |
| SPAST | Autosomal dominant spastic paraplegia-4 |
| SPEF2 | Primary ciliary dyskinesia; Spermatogenic failure-43 |
| SPEG | Centronuclear myopathy-5 |
| SPEG, ASIC4- | Centronuclear myopathy-5 |
| AS1 | |
| SPG11 | Amyotrophic lateral sclerosis type 5; Autosomal recessive hereditary |
| | spastic paraplegia; Spastic paraplegia-11 |
| SPG7 | Hereditary spastic paraplegia; Hereditary spastic paraplegia-7; |
| | Mitochondrial diseases |
| SPINK2 | Spermatogenic failure-29 |
| SPINK5 | Netherton syndrome |
| SPNS2 | Autosomal recessive deafness-115; Inborn genetic diseases |
| SPRTN | Ruijs-Aalfs syndrome |
| SPTA1 | Elliptocytosis-2; Hereditary pyropoikilocytosis |
| SPTB | Hereditary spherocytosis; Spherocytosis type 2 |
| SQSTM1 | Amyotrophic lateral sclerosis and/or frontotemporal dementia-1; Early- |
| | onset Paget disease of bone-2; SQSTM1-related disorder |
| SRCAP | Floating-Harbor syndrome |
| SRY | 46, XY sex reversal type 1 |
| ST14 | Autosomal recessive congenital ichthyosis-11 |
| STAG1 | Autosomal dominant mental retardation-47 |
| STAG3 | Abnormality of the ovary; Female infertility; Premature ovarian failure-8; |
| | Premature ovarian insufficiency |
| STAT1 | Autosomal recessive susceptibility to mycobacterial and viral infections |
| STIM1 | Combined immunodeficiency due to STIM1 deficiency; Tubular aggregate |
| | myopathy-1; Stormorken syndrome |
| STK11 | Hereditary cancer-predisposing syndrome; Peutz-Jeghers syndrome |
| STRA6 | Microphthalmia syndromic-9 |
| STRC | Autosomal recessive deafness-16; Rare genetic deafness |
| STXBP1 | Early infantile epileptic encephalopathy; Early infantile epileptic |
| | encephalopathy-4; Epileptic encephalopathy |
| STXBP2 | Familial hemophagocytic lymphohistiocytosis-5 |
| SUCLG1 | Encephalomyopathic mitochondrial DNA depletion syndrome-9 with |
| | methylmalonic aciduria |
| SUFU | Gorlin syndrome; Desmoplastic medulloblastoma; Medulloblastoma with |
| | extensive nodularity |
| SULT2B1 | Autosomal recessive congenital ichthyosis-14; Autosomal recessive |
| | congenital ichthyosis-2 |
| SUMF1 | Multiple sulfatase deficiency |
| SUN5 | Spermatogenic failure-16 |
| SURF1 | Abnormal pyramidal signs; Cerebellar ataxia; Charcot-Marie-Tooth disease |
| | type 4K; Dysarthria; Inborn genetic diseases; Leigh syndrome; Leigh |
| | syndrome due to COX IV deficiency; Leigh syndrome due to mitochondrial |
| | complex IV deficiency; Mitochondrial complex IV deficiency; Muscle |
| | weakness |
| SUZ12 | Imagawa-Matsumoto syndrome |
| SYCP2 | Cryptozoospermia; Early spermatogenesis maturation arrest; Oligosynaptic |
| | infertility |
| SYCP3 | Spermatogenic failure-4 |
| SYNE1 | Myogenic type arthrogryposis multiplex congenita; Cerebellar ataxia; |
| | Autosomal dominant Emery-Dreifuss muscular dystrophy-4; Autosomal |
| | recessive spinocerebellar ataxia-8 |
| SYNE4 | Rare genetic deafness |
| SYNGAP1 | Inborn genetic diseases; Autosomal dominant mental retardation-5 |
| SZT2 | Early infantile epileptic encephalopathy-18 |
| TAC3 | Hypogonadotropic hypogonadism-10 with or without anosmia |
| TACO1 | Mitochondrial complex IV deficiency |
| TALDO1 | Deficiency of transaldolase |
| TANGO2 | Acute rhabdomyolysis; Cardiac arrhythmia; Episodic flaccid weakness; |
| | Intellectual functioning disability; Seizures; Recurrent metabolic crises with |
| | rhabdomyolysis, cardiac arrhythmias, and neurodegeneration |
| TAP1 | Bare lymphocyte syndrome type 1 |
| TAP2 | Bare lymphocyte syndrome type 1; Peptide transporter PSF2 polymorphism |
| TAZ | 3-Methylglutaconic aciduria type 2 |
| TBC1D20 | Warburg micro syndrome-4 |
| TBC1D24 | DOORS syndrome caused by mutation in the TBC1 domain family member |
| | 24; Early infantile epileptic encephalopathy-1; Inborn genetic diseases; |
| | Autosomal dominant deafness-65 |
| TBCK | Inborn genetic diseases; Syndromic infantile encephalopathy; Infantile |
| | hypotonia with psychomotor retardation and characteristic facies-3 |
| TBR1 | Susceptibility to autism-5; Autistic behavior; Intellectual disability; |
| | Moderate global developmental delay; Neurodevelopmental disorder; |
| | Severe global developmental delay |
| TBX19 | Adrenocorticotropic hormone deficiency |
| TBX22 | Cleft palate with ankyloglossia |
| TBX3 | Ulnar-mammary syndrome |
| TBX4 | Coxopodopatellar syndrome |
| TBX5 | Variable congenital heart disease; Holt-Oram syndrome |
| TBXAS1 | Ghosal hematodiaphyseal dysplasia; Thromboxane synthetase deficiency |
| TCAP | Autosomal recessive limb-girdle muscular dystrophy type 2G; Dilated |
| | cardiomyopathy-1N; Primary familial hypertrophic cardiomyopathy |
| TCF12 | Craniosynostosis-3 |
| TCF20 | Neurodevelopmental abnormality |
| TCF4 | Intellectual disability; Pitt-Hopkins syndrome |
| TCN2 | Inborn genetic diseases; Transcobalamin II deficiency |
| TCOF1 | Treacher Collins syndrome-1 |
| TCTEX1D2 | Short-rib thoracic dysplasia-17 with or without polydactyly |
| TCTEX1D2, | Short-rib thoracic dysplasia-17 with or without polydactyly |
| TM4SF19- | |
| TCTEX1D2 | |
| TCTN2 | Joubert syndrome; Meckel syndrome type 8 |
| TCTN3 | Orofacial-digital syndrome-IV |
| TDO2 | Familial hypertryptophanemia |
| TDRD7 | Autosomal recessive congenital cataract-4 |
| TDRD9 | Spermatogenic failure-30 |
| TECPR2 | Autosomal recessive spastic paraplegia-49 |
| TECTA | Autosomal dominant nonsyndromic hearing loss and deafness-12; Rare |
| | genetic deafness; Autosomal recessive deafness-21; Neurosensory |
| | autosomal recessive deafness-21 |
| TENM3 | Syndromic microphthalmia-15 |
| TENT5A | Osteogenesis imperfecta type 18 |
| TEX14 | Spermatogenic failure-23 |
| TEX15 | Spermatogenic failure-25 |
| TFAP2B | Patent ductus arteriosus-2 |
| TFR2 | Hemochromatosis type 3 |
| TG | Iodotyrosyl coupling defect |
| TGFB2 | Cardiovascular phenotype; Holt-Oram syndrome; Loeys-Dietz syndrome-4 |
| TGFB3 | Cardiovascular phenotype; Loeys-Dietz syndrome-5 |
| TGFBR1 | Familial thoracic aortic aneurysm and aortic dissection |
| TGFBR2 | Familial thoracic aortic aneurysm and aortic dissection; Hereditary |
| | nonpolyposis colorectal cancer type 6; Loeys-Dietz syndrome; Loeys-Dietz |
| | syndrome-2; Malignant tumor of esophagus |
| TGM1 | Autosomal recessive congenital ichthyosis-1; Ichthyosis |
| TGM5 | Peeling skin syndrome-2 |
| TH | Autosomal recessive Segawa syndrome |
| THRB | Autosomal dominant generalized thyroid hormone resistance |
| TICAM1 | Susceptibility to herpes simplex encephalitis-4 |
| TIMM8A | Deafness dystonia syndrome |
| TIMMDC1 | Leigh syndrome |
| TJP2 | Progressive familial intrahepatic cholestasis-4 |
| TK2 | Mitochondrial DNA depletion syndrome-2 |
| TLR5 | Legionellosis; Melioidosis; Susceptibility to systemic lupus erythematosus- |
| | 1 |
| TM4SF20 | Specific language impairment-5 |
| TMC1 | Rare genetic deafness; Autosomal recessive nonsyndromic hearing loss-7 |
| TMCO1 | Craniofacial dysmorphism, skeletal anomalies, and mental retardation |
| | syndrome |
| TMCO6, | Cystic leukoencephalopathy |
| NDUFA2 | |
| TMEM127 | Hereditary paraganglioma-pheochromocytoma syndromes; Hereditary |
| | cancer-predisposing syndrome; Pheochromocytoma |
| TMEM216 | Joubert syndrome; Joubert syndrome-2; Meckel syndrome type 2; |
| | TMEM216-related disorders |
| TMEM237 | Joubert syndrome |
| TMEM260 | Structural heart defects and renal anomalies syndrome |
| TMEM67 | Cerebellar vermis hypoplasia; Generalized hypotonia; Iris coloboma; |
| | Joubert syndrome; Joubert syndrome-6; Meckel syndrome type 3; Meckel- |
| | Gruber syndrome; Nystagmus; TMEM67-related disorders |
| TMEM70 | Mitochondrial proton-transporting ATP synthase complex deficiency; |
| | Nuclearly-encoded mitochondrial complex V (ATP synthase) deficiency-2 |
| TMEM94 | Intellectual developmental disorder with cardiac defects and dysmorphic |
| | facies |
| TMEM99, | Bullous ichthyosiform erythroderma |
| KRT10 | |
| TMPRSS3 | Inborn genetic diseases; Rare genetic deafness; Autosomal recessive |
| | deafness-8 |
| TNFRSF10B | Squamous cell carcinoma of the head and neck |
| TNFRSF11B | Hyperphosphatasemia with bone disease |
| TNFRSF13B | Absent epiphyses; Chronic lung disease; Cleft palate; Clubfoot; Coat hanger |
| | sign of ribs; Common variable immune deficiency; Common variable |
| | immunodeficiency-2; Hemivertebrae; Immunoglobulin A deficiency-2; |
| | Interstitial pulmonary abnormality; Micrognathia; Patent ductus arteriosus; |
| | Preaxial foot polydactyly; Pseudoarthrosis; Respiratory failure; Short |
| | femur; Skeletal dysplasia; Vertebral hypoplasia; Vertebral segmentation |
| | defect |
| TNFRSF1A | Familial Periodic Fever; Susceptibility to multiple sclerosis-5 |
| TNFSF11 | Autosomal recessive osteopetrosis-2 |
| TNNI3 | Cardiovascular phenotype |
| TNNI3K, FPGT- | Cardiac conduction disease with or without dilated cardiomyopathy |
| TNNI3K | |
| TNNT2 | Cardiomyopathy; Cardiovascular phenotype; Familial hypertrophic |
| | cardiomyopathy-2; Familial restrictive cardiomyopathy-3; Hypertrophic |
| | cardiomyopathy; Left ventricular noncompaction-6; Primary familial |
| | hypertrophic cardiomyopathy |
| TNPO3 | Limb-girdle muscular dystrophy type 1F |
| TNXB | Classic-like Ehlers-Danlos syndrome-1; Ehlers-Danlos syndrome due to |
| | tenascin-X deficiency |
| TONSL | Sponastrime dysplasia |
| TONSL, | Sponastrime dysplasia |
| TONSL-AS1 | |
| TOP3A | Microcephaly, growth restriction, and increased sister chromatid exchange- |
| | 2 |
| TOPORS | Retinal dystrophy; Retinitis pigmentosa |
| TP53 | Head and neck neoplasms; Hereditary cancer-predisposing syndrome; Li- |
| | Fraumeni syndrome; Li-Fraumeni syndrome-1; Li-Fraumeni-like syndrome; |
| | Multiple myeloma; Neoplasm of the large intestine; Ovarian neoplasms |
| TP63 | Ectrodactyly, ectodermal dysplasia, and cleft lip/palate syndrome-3; |
| | Orofacial cleft-8 |
| TPI1 | Triosephosphate isomerase deficiency |
| TPM2 | Distal arthrogryposis type 2B4 |
| TPO | Deficiency of iodide peroxidase |
| TPP1 | Ceroid lipofuscinosis neuronal-2; Childhood-onset autosomal recessive |
| | slowly progressive spinocerebellar ataxia; Inborn genetic diseases; |
| | Neuronal ceroid lipofuscinosis |
| TPRN | Autosomal recessive deafness-79 |
| TRAPPC11 | Limb-girdle muscular dystrophy type 2S |
| TRAPPC2 | Spondyloepiphyseal dysplasia tarda |
| TRDN | Catecholaminergic polymorphic ventricular tachycardia-5; |
| | Catecholaminergic polymorphic ventricular tachycardia with or without |
| | muscle weakness |
| TREX1, ATRIP, | Aicardi Goutieres syndrome-1; Chilblain lupus; Retinal vasculopathy with |
| ATRIP-TREX1 | cerebral leukoencephalopathy and systemic manifestations; TREX1-related |
| | disorders |
| TRIM14, NANS | Genevieve type spondyloepimetaphyseal dysplasia |
| TRIM32, | Limb-girdle muscular dystrophy |
| ASTN2 | |
| TRIOBP | Nonsyndromic hearing loss and deafness |
| TRIP11 | Achondrogenesis type IA; Goldblatt hypertension; Osteochondrodysplasia |
| TRMU | Acute infantile liver failure due to synthesis defect of mtDNA-encoded |
| | proteins |
| TRNT1 | Retinitis pigmentosa and erythrocytic microcytosis; Sideroblastic anemia |
| | with B-cell immunodeficiency, periodic fevers, and developmental delay |
| TRPM4 | Cardiomyopathy; Progressive familial heart block type IB; TRPM4-related |
| | disorders |
| TRPS1 | Trichorhinophalangeal dysplasia type I |
| TRPV4 | Charcot-Marie-Tooth disease axonal type 2C |
| TRPV6 | Transient neonatal hyperparathyroidism |
| TSC1 | Cortical dysplasia; Cortical tubers; Focal cortical dysplasia type II; |
| | Hereditary cancer-predisposing syndrome; Lymphangiomyomatosis; |
| | Multiple renal cysts; Renal cortical cysts; Renal insufficiency; Seizures; |
| | Tuberous sclerosis-1; Tuberous sclerosis syndrome; Urinary bladder cancer |
| TSC2 | Focal cortical dysplasia type II; Lymphangiomyomatosis; Tuberous |
| | sclerosis-2; Tuberous sclerosis syndrome |
| TSFM | Combined oxidative phosphorylation deficiency-3; Primary dilated |
| | cardiomyopathy |
| TSHB | Secondary hypothyroidism |
| TSHR | Congenital nongoitrous hypothyroidism-1 |
| TSHZ1 | Congenital aural atresia |
| TSPAN1, | Congenital muscular alpha-dystroglycanopathy with brain and eye |
| POMGNT1 | anomalies; Congenital muscular dystrophy-dystroglycanopathy with mental |
| | retardation type B3; Limb-girdle muscular dystrophy-dystroglycanopathy |
| | type C3; Muscle eye brain disease; POMGNT1-related disorders; Retinitis |
| | pigmentosa-76 |
| TSPAN12 | Exudative vitreoretinopathy-5 |
| TSPAN7 | X-linked mental retardation-58 |
| TSPEAR | Hair/tooth type ectodermal dysplasia-14 with hypohidrosis |
| TSPEAR-AS1, | Hair/tooth type ectodermal dysplasia-14 with hypohidrosis; Autosomal |
| TSPEAR | recessive deafness-98 |
| TTC19 | Nuclear type 2 mitochondrial complex III deficiency |
| TTC21A | Spermatogenic failure-37 |
| TTC21B, | Short-rib thoracic dysplasia-4 with polydactyly |
| TTC21B-AS1 | |
| TTC29 | Spermatogenic failure-42 |
| TTC37 | Trichohepatoenteric syndrome; Trichohepatoenteric syndrome-1 |
| TTC7A | Multiple gastrointestinal atresias |
| TTLL5 | Cone-rod dystrophy-19 |
| TTN | Cardiomyopathy; Cardiovascular phenotype; Dilated cardiomyopathy-1G; |
| | Limb-girdle muscular dystrophy type 2J; Myotubular myopathy; Primary |
| | dilated cardiomyopathy; Tibial muscular dystrophy |
| TTN-AS1, TTN | Cardiovascular phenotype; Dilated cardiomyopathy-1G; Limb-girdle |
| | muscular dystrophy type 2J; Primary dilated cardiomyopathy; TTN-related |
| | disorders |
| TTN, | Primary dilated cardiomyopathy |
| LOC101927055 | |
| TTN, TTN-AS1 | Broad-based gait; Cardiomyopathy; Cardiovascular phenotype; Congenital |
| | muscular dystrophy; Decreased patellar reflex; Delayed gross motor |
| | development; Dilated cardiomyopathy-1G; Dilated cardiomyopathy-1S; |
| | Distal muscle weakness; Familial dilated cardiomyopathy; Familial |
| | hypertrophic cardiomyopathy-9; Gowers sign; Heart murmur; Limb-girdle |
| | muscular dystrophy type 2J; Muscular dystrophy; Myofibrillar myopathy-9 |
| | with early respiratory failure; Primary dilated cardiomyopathy; Proximal |
| | lower limb amyotrophy; Scoliosis; Severe muscular hypotonia; TTN-related |
| | disorder; Tibial muscular dystrophy; Waddling gait; Early-onset myopathy |
| | with fatal cardiomyopathy |
| TTPA | Familial isolated deficiency of vitamin E; Friedreich-like ataxia with |
| | isolated vitamin E deficiency |
| TUB, RIC3 | Retinal dystrophy and obesity |
| TUBA3D, | Keratoconus-9 |
| MZT2A | |
| TUBB8 | Oocyte maturation defect-2 |
| TULP1 | Leber congenital amaurosis; Retinitis pigmentosa |
| TWIST1 | Craniosynostosis-1; Robinow-Sorauf syndrome; Saethre-Chotzen syndrome |
| TXNL4A | Burn-Mckeown syndrome |
| TYK2 | Tyrosine kinase 2 deficiency |
| TYR | Ocular albinism with sensorineural deafness; Inborn genetic diseases; |
| | Myopia; Nonsyndromic oculocutaneous albinism; Nystagmus; |
| | Oculocutaneous albinism; Oculocutaneous albinism type 1B; Variation in |
| | skin/hair/eye pigmentation-3; Tyrosinase-negative oculocutaneous albinism |
| TYRP1, | Oculocutaneous albinism type 3 |
| LURAP1L-AS1 | |
| UBAP1 | Autosomal dominant spastic paraplegia-80 |
| UBE3A, | Angelman syndrome; History of neurodevelopmental disorder; Inborn |
| SNHG14 | genetic diseases |
| UBE3B | Kaufman oculocerebrofacial syndrome |
| UBR1 | Johanson-Blizzard syndrome |
| UCP3 | Severe obesity and type II diabetes |
| UGT1A, | Crigler-Najjar syndrome type II; Crigler-Najjar syndrome type 1 |
| UGT1A10, | |
| UGT1A8, | |
| UGT1A7, | |
| UGT1A6, | |
| UGT1A5, | |
| UGT1A9, | |
| UGT1A4, | |
| UGT1A1, | |
| UGT1A3 | |
| UNC13D | Familial hemophagocytic lymphohistiocytosis-3 |
| UNC80 | Hypotonia-speech impairment-severe cognitive delay syndrome; Infantile |
| | hypotonia with psychomotor retardation and characteristic facies-2 |
| UNG | Hyper-IgM syndrome type 5 |
| UPF3B | X-linked syndromic mental retardation-14 |
| USH1C | Autosomal recessive deafness-18; Rare genetic deafness; Retinal dystrophy; |
| | Retinitis pigmentosa; Usher syndrome type 1C; Usher syndrome type 1 |
| USH2A | Abnormality of the upper limb; Abnormality of upper limb bone; |
| | Abnormality of upper limb joint; Anxiety; Brisk reflexes; Chronic pain; |
| | Cognitive impairment; Cone-rod dystrophy; Congenital sensorineural |
| | hearing impairment; Congenital stationary night blindness; Dislocated |
| | radial head; Distal arthrogryposis; Dysautonomia; Hearing impairment; |
| | High palate; Inborn genetic diseases; Macular dystrophy; Multiple joint |
| | contractures; Rare genetic deafness; Retinal dystrophy; Retinitis |
| | pigmentosa; Retinitis pigmentosa-39; Short stature; USH2A-related |
| | disorders; Usher syndrome type 2A; Usher syndrome type 2 |
| USH2A, | Rare genetic deafness; Retinal dystrophy; Retinitis pigmentosa-39; USH2A- |
| USH2A-AS1 | related disorders; Usher syndrome type 2A |
| USH2A, | Rare genetic deafness; Retinitis pigmentosa-39; Usher syndrome type 2A |
| USH2A-AS2 | |
| USP18 | Pseudo-TORCH syndrome-2 |
| USP27X | X-linked mental retardation-105 |
| USP9X | USP9X related disorders; X-linked female-restricted syndromic mental |
| | retardation-99 |
| VCL | Dilated cardiomyopathy-1W; Familial hypertrophic cardiomyopathy-15; |
| | Primary dilated cardiomyopathy |
| VHL | Familial erythrocytosis-2; Hereditary cancer-predisposing syndrome; Von |
| | Hippel-Lindau syndrome |
| VHL, | Familial erythrocytosis-2; Hereditary cancer-predisposing syndrome; |
| LOC107303340 | Papillary renal cell carcinoma-1; Von Hippel-Lindau syndrome |
| VIM, VIM-AS1 | Cataract-30; Congenital cataract |
| VIPAS39 | Arthrogryposis, renal dysfunction, and cholestasis-2 |
| VPS13A | Choreoacanthocytosis |
| VPS13B | Abnormality of the eye; Cohen syndrome; Inborn genetic diseases; |
| | Intellectual disability; Microcephaly; Neutropenia; Progressive visual loss; |
| | Recurrent aphthous stomatitis; Retinal dystrophy; Short foot; Short stature; |
| | Small hand |
| VPS33B | Arthrogryposis, renal dysfunction, and cholestasis-1; Inborn genetic |
| | diseases |
| VRK2, FANCL | Complementation group A Fanconi anemia; Complementation group L |
| | Fanconi anemia |
| VWF | von Willebrand disorder |
| WAC | Desanto-Shinawi syndrome |
| WAS | Wiskott-Aldrich syndrome; X-linked severe congenital neutropenia; X- |
| | linked thrombocytopenia with normal platelets |
| WDR35 | Cranioectodermal dysplasia; Cranioectodermal dysplasia-2; Jeune thoracic |
| | dystrophy; Short-rib thoracic dysplasia-7 without polydactyly; Short rib |
| | polydactyly syndrome; Short rib polydactyly syndrome-5; Digenic short-rib |
| | thoracic dysplasia-7/20 with polydactyly; WDR35-related disorders |
| WDR45 | Neurodegeneration with brain iron accumulation; Neurodegeneration with |
| | brain iron accumulation-5 |
| WDR72 | Amelogenesis imperfecta |
| WDR73 | Galloway-Mowat syndrome-1 |
| WEE2-AS1, | Oocyte maturation defect-5 |
| WEE2 | |
| WFS1 | Autosomal dominant nonsyndromic deafness-6; Diabetes mellitus and |
| | insipidus with optic atrophy and deafness; WFS1-related spectrum |
| | disorders; Wolfram-like syndrome |
| WHRN | Autosomal recessive deafness-31; Rare genetic deafness; Usher syndrome |
| | type 2D |
| WRN | Medulloblastoma; Werner syndrome |
| WT1 | Drash syndrome; Frasier syndrome; Wilms tumor, aniridia, genitourinary |
| | anomalies, and mental retardation syndrome; Wilms tumor-1 |
| WT1, | Drash syndrome; Frasier syndrome; Pre-B-cell acute lymphoblastic |
| LOC107982234 | leukemia; Wilms tumor, aniridia, genitourinary anomalies, and mental |
| | retardation syndrome; Wilms tumor-1 |
| XDH | Deficiency of xanthine oxidase |
| XIAP | X-linked lymphoproliferative syndrome-2 |
| XK | McLeod neuroacanthocytosis syndrome |
| XPA | Xeroderma pigmentosum; Xeroderma pigmentosum group A |
| XPC | Xeroderma pigmentosum group C |
| XRCC2 | Complementation group U Fanconi anemia; Hereditary cancer syndrome; |
| | Hereditary breast and ovarian cancer syndrome; Hereditary cancer- |
| | predisposing syndrome; Ovarian neoplasms |
| XRCC4 | Short stature, microcephaly, and endocrine dysfunction |
| XYLT1 | Desbuquois dysplasia-2 |
| XYLT1, | Desbuquois dysplasia-2 |
| LOC102723692 | |
| XYLT2 | Inborn genetic diseases; Autosomal recessive spondyloocular syndrome |
| YY1AP1 | Grange syndrome |
| ZBTB18 | Autosomal dominant mental retardation-22 |
| ZDBF2 | Nasopalpebral lipoma-coloboma syndrome |
| ZEB2 | Mowat-Wilson syndrome |
| ZFYVE26 | Hereditary spastic paraplegia-15; Spastic paraplegia |
| ZFYVE26, | Abnormality of the eye; Leber congenital amaurosis-13; RDH12-related |
| RDH12 | disorders; Retinal dystrophy; Retinitis pigmentosa |
| ZMPSTE24 | Lethal tight skin contracture syndrome; Mandibuloacral dysplasia with type |
| | B lipodystrophy; ZMPSTE24-related disorders |
| ZNF408 | Retinitis pigmentosa-72 |
| ZNF462 | Craniosynostosis; Mental retardation; Weiss-Kruszka syndrome |
| ZNF711 | ZNF711-related X-linked mental retardation |
| ZP1 | Oocyte maturation defect-1 |
| ZP2 | Oocyte maturation defect-6 |

In an embodiment, the disease or disorder associated with a PTC is a lysosomal storage disease (e.g., Fabry disease, Gaucher disease, or Niemann-Pick disease). In some embodiments, the disease or disorder associated with a PTC is Fabry disease. In an embodiment, upon administration of a TREM (e.g., a TREM described herein) to a cell or subject, the level of a GLA protein in the cell or subject is modulated, e.g., increased, by about 0.1%, 0.500, 100, 200, 3%, 4% 5%, 6%, 7%, 8%, 9%, 10%, 15%, 20%, 25%30%, 35%, 40%, 45%, 50%, 55%, 60%, 65%, 70%, 75%, 80%, 85%, 900%, 9500 or more, e.g., compared with a reference value (e.g., level of a GLA protein in a healthy, non-Fabry disease fibroblast).

In some embodiments, the disease or disorder associated with a PTC is a blood clotting disorder, e.g., Hemophilia B. In an embodiment, upon administration of a TREM (e.g., a TREM described herein) to a cell or subject, the level of a Factor IX (FIX) protein in the cell or subject is modulated, e.g., increased, by about 0.1%, 0.5%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or more, e.g., compared with a reference value (e.g., level of a FIX protein in a healthy, non-disease cell).

In some embodiments, the disease or disorder associated with a PTC is an autosomal recessive disorder, such as neuronal ceroid lipofuscinosis type 2 (CNL2). In an embodiment, upon administration of a TREM (e.g., a TREM described herein) to a cell or subject, the level of a tripeptidyl peptidase 1 (TPP1) protein in the cell or subject is modulated, e.g., increased, by about 0.1%, 0.5%, 1%, 2%, 3%, 4% 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or more, e.g., compared with a reference value (e.g., level of a TPP1 protein in a healthy, non-disease cell).

In some embodiments, the disease or disorder associated with a PTC is a disease or disorder associated with hearing loss, such as Usher syndrome (e.g., Usher syndrome type 1 F). In an embodiment, upon administration of a TREM (e.g., a TREM described herein) to a cell or subject, the level of a protocadherin 15 precursor (PCDH15) protein in the cell or subject is modulated, e.g., increased, by about 0.1%, 0.5%, 1%, 2%, 3%, 4% 5%, 6%, 7%, 8% 9%, 10%, 15%, 20%, 25%, 30%, 35%, 40%, 45%, 50%, 55%, 60%, 65%, 70%, 75%, 80%, 85%, 90%, 95%, or more, e.g., compared with a reference value (e.g., level of a PCDH15 protein in a healthy, non-disease cell).

In an embodiment, the disease or disorder associated with a PTC is a proliferative disease, such as a benign neoplasm or a cancer. In an embodiment, the proliferative disease is associated with a benign neoplasm. For example, a benign neoplasm may include adenoma, fibroma, hemangioma, tuberous sclerosis, and lipoma. All types of benign neoplasms disclosed herein or known in the art are contemplated as being within the scope of the disclosure.

In an embodiment, the proliferative disease is a cancer. As used herein, the term “cancer” refers to a malignant neoplasm (Stedman's Medical Dictionary, 25th ed.; Hensyl ed.; Williams & Wilkins: Philadelphia, 1990). All types of cancers disclosed herein or known in the art are contemplated as being within the scope of the disclosure. Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; eye cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer), e.g., adenoid cystic carcinoma (ACC)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenstrom's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendocrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic adenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva). In some embodiments, the cancer is a solid tumor, such as a sarcoma or a carcinoma (e.g., lung cancer, brain cancer, breast cancer, bladder cancer, prostate cancer, colon cancer, rectal cancer).

In another aspect, the present disclosure features methods of treating a disease or disorder in a cell or subject by administration of a TREM (e.g., a TREM described herein) to the cell or subject. Exemplary diseases or disorders include hemophilias, aminoacidopathies, metal storage disorders, perox isome biogenesis disorder, progressive rare lung disease, diseases related to lipid metabolism, diseases related to galactose metabolism, systemic organic acidemias, urea cycle disorders, cholestastis disorders, bilirubin metabolism disorders, lysososomal storage disorders, glycogen storage diseases, and oxalate metabolism disorders. In an embodiment, the disease or disorder is a hemophilia, e.g., hemophilia A or hemophilia B. In an embodiment, the disease or disorder is an aminoacidopathy, e.g., tyrosinemia type 1, tyrosinemia type 2, tyrosinemia type 3, maple syrup urine disease, alkaptonuria, or phenylketonuria. In an embodiment, the disease or disorder is a systemic organic acidemia, e.g., methylmalonic acidemia (MMUT), methylmalonic acidemia (non-MMUT), propionic acidemia type A, propionic acidemia type B, or isovaleric acidemia. In an embodiment, the disease or disorder is a urea cycle disorder, e.g, argininosuccinate lyase deficiency, argininosuccinate lyase deficiency-D, citrullinemia type 1, citrullinemia type 2, carbamoyl phosphate synthetase-D, ornithine transcarbamylase, arginemia, or hyperornithinemia-hyperammonemia-homocitrullinuria (HIHH) syndrome. In an embodiment, the disease or disorder is lysosomal storage disorder, e.g., mucopolysaccharidosis 1, mucopolysaccharidosis 2, Fabry disease, lysosomal acid lipas deficiency, Pompe disease, Gaucher disease, Niemann Pick A, or Niemann Pick B. In an embodiment, the disease or disorder is a bilirubin metabolism disorder, e.g., Crigler-Najjar syndrome. In an embodiment, the disease or disorder is a cholestastis disorder, e.g., progressive familial intrahepatic cholestasis (PFIC) type 1, PFIC type 2, or PFIC type 3. In an embodiment, the disease or disorder is a disease related to lipid metabolism, e.g., sitosterolemia (ABCG5) or sitosterolemia (ABCG8). In an embodiment, the disease or disorder is a glycogen storage disease, e.g., glycogen storage disease 1a, glycogen storage disease 1b, or glycogen storage disease 3a. In an embodiment, the disease or disorder is a metal storage disorder, e.g., Wilson disease or hereditary hemochromatosis. In an embodiment, the disease or disorder is a progressive rare lung disease, e.g., alpha-1 antitrypsin deficiency. In an embodiment, the disease or disorder is a perox isome biogenesis disorder, e.g., PBD RCDP1. In an embodiment, the disease or disorder is an oxalate metabolism disorder, e.g, primary hyperoxaluria type 1, primary hyperoxaluria type 2, or primary hyperoxaluria type 3. In an embodiment, the disease or disorder is a congenital disorder related to Notch signaling, e.g., Alagille syndrome. In an embodiment, the disease or disorder is an amyloidosis, e.g., familial amyloid polyneuropathy.

In one aspect, the present disclosure features a method of treating a disease or disorder in a subject, the method comprising administering to the subject a TREM comprising the nucleotide sequence of any one of the TREMS listed in FIG. 3. In an embodiment, the disease or disorder is selected from a hemophilia, aminoacidopathy, metal storage disorder, perox isome biogenesis disorder, progressive rare lung disease, disease related to lipid metabolism, disease related to galactose metabolism, systemic organic acidemia, urea cycle disorder, cholestastis disorder, bilirubin metabolism disorder, lysososomal storage disorder, glycogen storage disease, and oxalate metabolism disorder. In an embodiment, the TREM comprises the sequence of any one of SEQ ID NO: 622, 623, 624, 4249, 4386, 4834, 5630, 6707, 6749, 6947, or 8051, or a fragment or variant thereof.

### Method of Making TREMs, TREM Core Fragments, and TREM Fragments

In vitro methods for synthesizing oligonucleotides are known in the art and can be used to make a TREM, a TREM core fragment or a TREM fragment disclosed herein. For example, a TREM, TREM core fragment or TREM fragment can be synthesized using solid state synthesis or liquid phase synthesis.

In an embodiment, a TREM, a TREM core fragment or a TREM fragment made according to an in vitro synthesis method disclosed herein has a different modification profile compared to a TREM expressed and isolated from a cell, or compared to a naturally occurring tRNA.

An exemplary method for making a modified TREM is provided in Example 1. The method provided in Example 1 can also be used to make a synthetic TREM core fragment or synthetic TREM fragment. Additional synthetic methods are disclosed in Hartsel S A et al., (2005) Oligonucleotide Synthesis, 033-050, the entire contents of which are hereby incorporated by reference.

### TREM Composition

In an embodiment, a TREM composition, e.g., a TREM pharmaceutical composition, comprises a pharmaceutically acceptable excipient. Exemplary excipients include those provided in the FDA Inactive Ingredient Database (https://www.accessdata.fda.gov/scripts/cder/iig/index. Cfm).

In an embodiment, a TREM composition, e.g., a TREM pharmaceutical composition, comprises at least 1, 2, 3, 4, 5, 6, 7, 8, 9, 10, 15, 20, 30, 40, 50, 60, 70, 80, 90, 100 or 150 grams of TREM, TREM core fragment or TREM fragment. In an embodiment, a TREM composition, e.g., a TREM pharmaceutical composition, comprises at least 1, 2, 3, 4, 5, 6, 7, 8, 9, 10, 15, 20, 30, 40, 50 or 100 milligrams of TREM, TREM core fragment or TREM fragment.

In an embodiment, a TREM composition, e.g., a TREM pharmaceutical composition, is at least 10, 20, 30, 40, 50, 60, 70, 80, 90, 95 or 99% dry weight TREMs, TREM core fragments or TREM fragments.

In an embodiment, a TREM composition comprises at least 1×10⁶ TREM molecules, at least 1×10⁷ TREM molecules, at least 1×10⁸ TREM molecules or at least 1×10⁹ TREM molecules.

In an embodiment, a TREM composition comprises at least 1×10⁶ TREM core fragment molecules, at least 1×10⁷ TREM core fragment molecules, at least 1×10⁸ TREM core fragment molecules or at least 1×10⁹ TREM core fragment molecules.

In an embodiment, a TREM composition comprises at least 1×10⁶ TREM fragment molecules, at least 1×10⁷ TREM fragment molecules, at least 1×10⁸ TREM fragment molecules or at least 1×10⁹ TREM fragment molecules.

In an embodiment, a TREM composition produced by any of the methods of making disclosed herein can be charged with an amino acid using an in vitro charging reaction as known in the art.

In an embodiment, a TREM composition comprise one or more species of TREMs, TREM core fragments, or TREM fragments. In an embodiment, a TREM composition comprises a single species of TREM, TREM core fragment, or TREM fragment. In an embodiment, a TREM composition comprises a first TREM, TREM core fragment, or TREM fragment species and a second TREM, TREM core fragment, or TREM fragment species. In an embodiment, the TREM composition comprises X TREM, TREM core fragment, or TREM fragment species, wherein X=2, 3, 4, 5, 6, 7, 8, 9, or 10.

In an embodiment, the TREM, TREM core fragment, or TREM fragment has at least 70, 75, 80, 85, 90, or 95, or has 100%, identity with a sequence encoded by a nucleic acid in Table 1.

In an embodiment, the TREM comprises a consensus sequence provided herein.

A TREM composition can be formulated as a liquid composition, as a lyophilized composition or as a frozen composition.

In some embodiments, a TREM composition can be formulated to be suitable for pharmaceutical use, e.g., a pharmaceutical TREM composition. In an embodiment, a pharmaceutical TREM composition is substantially free of materials and/or reagents used to separate and/or purify a TREM, TREM core fragment, or TREM fragment.

In some embodiments, a TREM composition can be formulated with water for injection. In some embodiments, a TREM composition formulated with water for injection is suitable for pharmaceutical use, e.g., comprises a pharmaceutical TREM composition.

### TREM Characterization

A TREM, TREM core fragment, or TREM fragment, or a TREM composition, e.g., a pharmaceutical TREM composition, produced by any of the methods disclosed herein can be assessed for a characteristic associated with the TREM, TREM core fragment, or TREM fragment or the TREM composition, such as purity, sterility, concentration, structure, or functional activity of the TREM, TREM core fragment, or TREM fragment. Any of the above-mentioned characteristics can be evaluated by providing a value for the characteristic, e.g., by evaluating or testing the TREM, TREM core fragment, or TREM fragment, or the TREM composition, or an intermediate in the production of the TREM composition. The value can also be compared with a standard or a reference value. Responsive to the evaluation, the TREM composition can be classified, e.g., as ready for release, meets production standard for human trials, complies with ISO standards, complies with cGMP standards, or complies with other pharmaceutical standards. Responsive to the evaluation, the TREM composition can be subjected to further processing, e.g., it can be divided into aliquots, e.g., into single or multi-dosage amounts, disposed in a container, e.g., an end-use vial, packaged, shipped, or put into commerce. In embodiments, in response to the evaluation, one or more of the characteristics can be modulated, processed or re-processed to optimize the TREM composition. For example, the TREM composition can be modulated, processed or re-processed to (i) increase the purity of the TREM composition; (ii) decrease the amount of fragments in the composition; (iii) decrease the amount of endotoxins in the composition; (iv) increase the in vitro translation activity of the composition; (v) increase the TREM concentration of the composition; or (vi) inactivate or remove any viral contaminants present in the composition, e.g., by reducing the pH of the composition or by filtration.

In an embodiment, the TREM, TREM core fragment, or TREM fragment (e.g., TREM composition or an intermediate in the production of the TREM composition) has a purity of at least 30%, 40%, 50%, 60%, 70%, 80%, 85%, 90%, 91%, 92%, 93%, 94%, 95%, 96%, 97%, 98%, or 99%, i.e., by mass.

In an embodiment, the TREM (e.g., TREM composition or an intermediate in the production of the TREM composition) has less than 0.1%, 0.5%, 1%, 2%, 3%, 4%, 5%, 6%, 7%, 8%, 9%, 10%, 15%, 20%, 25% TREM fragments relative to full length TREMs.

In an embodiment, the TREM, TREM core fragment, or TREM fragment (e.g., TREM composition or an intermediate in the production of the TREM composition) has low levels or absence of endotoxins, e.g., a negative result as measured by the Limulus amebocyte lysate (LAL) test.

In an embodiment, the TREM, TREM core fragment, or TREM fragment (e.g., TREM composition or an intermediate in the production of the TREM composition) has in-vitro translation activity, e.g., as measured by an assay described in Examples 12-13.

In an embodiment, the TREM, TREM core fragment, or TREM fragment (e.g., TREM composition or an intermediate in the production of the TREM composition) has a TREM concentration of at least 0.1 ng/mL, 0.5 ng/mL, 1 ng/mL, 5 ng/mL, 10 ng/mL, 50 ng/mL, 0.1 ug/mL, 0.5 ug/mL, 1 ug/mL, 2 μg/mL, 5 μg/mL, 10 μg/mL, 20 μg/mL, 30 μg/mL, 40 μg/mL, 50 μg/mL, 60 μg/mL, 70 μg/mL, 80 μg/mL, 100 μg/mL, 200 μg/mL, 300 μg/mL, 500 μg/mL, 1000 μg/mL, 5000 μg/mL, 10,000 μg/mL, or 100,000 μg/mL.

In an embodiment, the TREM, TREM core fragment, or TREM fragment (e.g., TREM composition or an intermediate in the production of the TREM composition) is sterile, e.g., the composition or preparation supports the growth of fewer than 100 viable microorganisms as tested under aseptic conditions, the composition or preparation meets the standard of USP<71>, and/or the composition or preparation meets the standard of USP<85>.

In an embodiment, the TREM, TREM core fragment, or TREM fragment (e.g., TREM composition or an intermediate in the production of the TREM composition) has an undetectable level of viral contaminants, e.g., no viral contaminants. In an embodiment, any viral contaminant, e.g., residual virus, present in the composition is inactivated or removed. In an embodiment, any viral contaminant, e.g., residual virus, is inactivated, e.g., by reducing the pH of the composition. In an embodiment, any viral contaminant, e.g., residual virus, is removed, e.g., by filtration or other methods known in the field.

### TREM Administration

Any TREM composition or pharmaceutical composition described herein can be administered to a cell, tissue or subject, e.g., by direct administration to a cell, tissue and/or an organ in vitro, ex-vivo or in vivo. In-vivo administration may be via, e.g., by local, systemic and/or parenteral routes, for example intravenous, subcutaneous, intraperitoneal, intrathecal, intramuscular, ocular, nasal, urogenital, intradermal, dermal, enteral, intravitreal, intracerebral, intrathecal, or epidural.

### Vectors and Carriers

In some embodiments the TREM, TREM core fragment, or TREM fragment or TREM composition described herein, is delivered to cells, e.g. mammalian cells or human cells, using a vector. The vector may be, e.g., a plasmid or a virus. In some embodiments, delivery is in vivo, in vitro, ex vivo, or in situ. In some embodiments, the virus is an adeno associated virus (AAV), a lentivirus, or an adenovirus. In some embodiments, the system or components of the system are delivered to cells with a viral-like particle or a virosome. In some embodiments, the delivery uses more than one virus, viral-like particle or virosome.

### Carriers

A TREM, a TREM composition or a pharmaceutical TREM composition described herein may comprise, may be formulated with, or may be delivered in, a carrier.

### Viral Vectors

The carrier may be a viral vector (e.g., a viral vector comprising a sequence encoding a TREM, a TREM core fragment or a TREM fragment). The viral vector may be administered to a cell or to a subject (e.g., a human subject or animal model) to deliver a TREM, a TREM core fragment or a TREM fragment, a TREM composition or a pharmaceutical TREM composition.

A viral vector may be systemically or locally administered (e.g., injected). Viral genomes provide a rich source of vectors that can be used for the efficient delivery of exogenous genes into a mammalian cell. Viral genomes are known in the art as useful vectors for delivery because the polynucleotides contained within such genomes are typically incorporated into the nuclear genome of a mammalian cell by generalized or specialized transduction. These processes occur as part of the natural viral replication cycle, and do not require added proteins or reagents in order to induce gene integration. Examples of viral vectors include a retrovirus (e.g., Retroviridae family viral vector), adenovirus (e.g., Ad5, Ad26, Ad34, Ad35, and Ad48), parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g., measles and Sendai), positive strand RNA viruses, such as picornavirus and alphavirus, and double stranded DNA viruses including adenovirus, herpesvirus (e.g., Herpes Simplex virus types 1 and 2, Epstein-Barr virus, cytomegalovirus, replication deficient herpes virus), and poxvirus (e.g., vaccinia, modified vaccinia Ankara (MVA), fowlpox and canarypox). Other viruses include Norwalk virus, togavirus, flavivirus, reoviruses, papovavirus, hepadnavirus, human papilloma virus, human foamy virus, and hepatitis virus, for example. Examples of retroviruses include: avian leukosis-sarcoma, avian C-type viruses, mammalian C-type, B-type viruses, D-type viruses, oncoretroviruses, HTLV-BLV group, lentivirus, alpharetrovirus, gammaretrovirus, spumavirus (Coffin, J. M., Retroviridae: The viruses and their replication, Virology (Third Edition) Lippincott-Raven, Philadelphia, 1996). Other examples include murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, in U.S. Pat. No. 5,801,030, the teachings of which are incorporated herein by reference. In some embodiments the system or components of the system are delivered to cells with a viral-like particle or a virosome.

### Cell and Vesicle-Based Carriers

A TREM, a TREM core fragment or a TREM fragment, a TREM composition or a pharmaceutical TREM composition described herein can be administered to a cell in a vesicle or other membrane-based carrier.

In embodiments, a TREM, a TREM core fragment or a TREM fragment, or TREM composition, or pharmaceutical TREM composition described herein is administered in or via a cell, vesicle or other membrane-based carrier. In one embodiment, the TREM, TREM core fragment, TREM fragment, or TREM composition or pharmaceutical TREM composition can be formulated in liposomes or other similar vesicles. Liposomes are spherical vesicle structures composed of a uni- or multilamellar lipid bilayer surrounding internal aqueous compartments and a relatively impermeable outer lipophilic phospholipid bilayer. Liposomes may be anionic, neutral or cationic. Liposomes are biocompatible, nontoxic, can deliver both hydrophilic and lipophilic drug molecules, protect their cargo from degradation by plasma enzymes, and transport their load across biological membranes and the blood brain barrier (BBB) (see, e.g., Spuch and Navarro, Journal of Drug Delivery, vol. 2011, Article ID 469679, 12 pages, 2011. doi:10.1155/2011/469679 for review).

Vesicles can be made from several different types of lipids; however, phospholipids are most commonly used to generate liposomes as drug carriers. Methods for preparation of multilamellar vesicle lipids are known in the art (see for example U.S. Pat. No. 6,693,086, the teachings of which relating to multilamellar vesicle lipid preparation are incorporated herein by reference). Although vesicle formation can be spontaneous when a lipid film is mixed with an aqueous solution, it can also be expedited by applying force in the form of shaking by using a homogenizer, sonicator, or an extrusion apparatus (see, e.g., Spuch and Navarro, Journal of Drug Delivery, vol. 2011, Article ID 469679, 12 pages, 2011. doi:10.1155/2011/469679 for review). Extruded lipids can be prepared by extruding through filters of decreasing size, as described in Templeton et al., Nature Biotech, 15:647-652, 1997, the teachings of which relating to extruded lipid preparation are incorporated herein by reference.

### Lipid Nanoformulations/Lipid-Based Carriers

Lipid nanoparticles are another example of a carrier that provides a biocompatible and biodegradable delivery system for the TREM, TREM core fragment, TREM fragment, or TREM composition or pharmaceutical TREM composition described herein. Nanostructured lipid carriers (NLCs) are modified solid lipid nanoparticles (SLNs) that retain the characteristics of the SLN, improve drug stability and loading capacity, and prevent drug leakage. Polymer nanoparticles (PNPs) are an important component of drug delivery. These nanoparticles can effectively direct drug delivery to specific targets and improve drug stability and controlled drug release. Lipid-polymer nanoparticles (PLNs), a new type of carrier that combines liposomes and polymers, may also be employed. These nanoparticles possess the complementary advantages of PNPs and liposomes. A PLN is composed of a core-shell structure; the polymer core provides a stable structure, and the phospholipid shell offers good biocompatibility. As such, the two components increase the drug encapsulation efficiency rate, facilitate surface modification, and prevent leakage of water-soluble drugs. For a review, see, e.g., Li et al. 2017, Nanomaterials 7, 122; doi:10.3390/nano7060122.

Exemplary lipid nanoparticles are disclosed in International Application PCT/US2014/053907, the entire contents of which are hereby incorporated by reference. For example, an LNP described in paragraphs [403-406] or [410-413] ofPCT/US2014/053907 can be used as a carrier for the TREM, TREM core fragment, TREM fragment, or TREM composition or pharmaceutical TREM composition described herein.

Additional exemplary lipid nanoparticles are disclosed in U.S. Pat. No. 10,562,849 the entire contents of which are hereby incorporated by reference. For example, an LNP of formula (I) as described in columns 1-3 of U.S. Pat. No. 10,562,849 can be used as a carrier for the TREM, TREM core fragment, TREM fragment, or TREM composition or pharmaceutical TREM composition described herein.

Lipids that can be used in nanoparticle formations (e.g., lipid nanoparticles) include, for example those described in Table 4 of WO2019217941, which is incorporated by reference, e.g., a lipid-containing nanoparticle can comprise one or more of the lipids in Table 4 of WO2019217941. Lipid nanoparticles can include additional elements, such as polymers, such as the polymers described in Table 5 of WO2019217941, incorporated by reference.

In some embodiments, conjugated lipids, when present, can include one or more of PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-0-(2′,3′-di(tetradecanoyloxy)propyl-1-0(w-methoxy(polyethoxy)ethyl) butanedioate (PEG-S-DMG)), PEG dialkoxypropylcarbam, N-(carbonyl-methoxypoly ethylene glycol 2000)-1,2-distearoyl-sn-glycero-3-phosphoethanolamine sodium salt, and those described in Table 2 of WO2019051289 (incorporated by reference), and combinations of the foregoing.

In some embodiments, sterols that can be incorporated into lipid nanoparticles include one or more of cholesterol or cholesterol derivatives, such as those in WO2009/127060 or US2010/0130588, which are incorporated by reference. Additional exemplary sterols include phytosterols, including those described in Eygeris et al (2020), incorporated herein by reference.

### Cationic Lipids (Positively Charged) and Ionizable Lipids

In some embodiments, the lipid-based carrier (or lipid nanoformulation) comprises one or more cationic lipids, e.g., a cationic lipid that can ex ist in a positively charged or neutral form depending on pH, or an amine-containing lipid that can be readily protonated. In some embodiments, the cationic lipid is a lipid capable of being positively charged, e.g., under physiological conditions.

Exemplary cationic lipids include one or more amine group(s) which bear the positive charge. Examples of positively charged (cationic) lipids include, but are not limited to, N,N-dimethyl-N,N-dioctacyl ammonium bromide (DDAB) and chloride DDAC), N-(1-(2,3-dioleyloxy)propyl)-N,N,N-trimethylammonium chloride (DOTMA), 3β-[N(N,N-dimethylaminoethyl)carbamoyl) cholesterol (DC-chol), 1,2-dioleoyloxy-3-[trimethylammonio]-propane (DOTAP), 1,2-dioctadecyloxy-3-[trimethylammonio]-propane (DSTAP), and 1,2-dioleoyloxypropyl-3-dimethyl-hydroxy ethyl ammonium chloride (DORI), N,N-dioleyl-N,N-dimethyliammonium chloride (DODAC), N,N-dimethyl-2,3-dioleyloxy)propylamine (DODNMA), 1,2-Dioleoyl-3-Dimethylammonium-propane (DODAP), 1,2-Dioleoylcarbarnyl-3-Dimethylammonium-propane (DOCDAP). 1,2-Dilineoyl-3-Dimethylammonium-propane (DLINDAP), 3-Dimethylamino-2-(Cholest-5-en-3-beta-oxy butan-4-oxy)-1-(cis,cis-9,12-octadecadienoxy)propane (CLinDMA), 2-[5′-(cholest-5-en-3-beta-oxy)-3′oxapentoxy)-3-dimethyl-1-(cis, cis-9′,12-octadecadienoxy)propane CpLin DMA), N,N-Dimethyl-3,4-dioleyloxybenzylamine (DMOBA), and the cationic lipids described in e.g. Martin et al., Current Pharmaceutical Design, pages 1-394, which is herein incorporated by reference in its entirety. In some embodiments, the lipid-based carrier (or lipid nanoformulation) comprises more than one cationic lipid.

In some embodiments, the lipid-based carrier (or lipid nanoformulation) comprises a cationic lipid having an effective pKa over 6.0. In some embodiments, the lipid-based carrier (or lipid nanoformulation) further comprises a second cationic lipid having a different effective pKa (e.g., greater than the first effective pKa) than the first cationic lipid.

In some embodiments, cationic lipids that can be used in the lipid-based carrier (or lipid nanoformulation) include, for example those described in Table 4 of WO 2019/217941, which is incorporated by reference.

In some embodiments, the cationic lipid is an ionizable lipid (e.g., a lipid that is protonated at low pH, but that remains neutral at physiological pH). In some embodiments, the lipid-based carrier (or lipid nanoformulation) may comprise one or more additional ionizable lipids, different than the ionizable lipids described herein, Exemplary ionizable lipids include, but are not limited to,

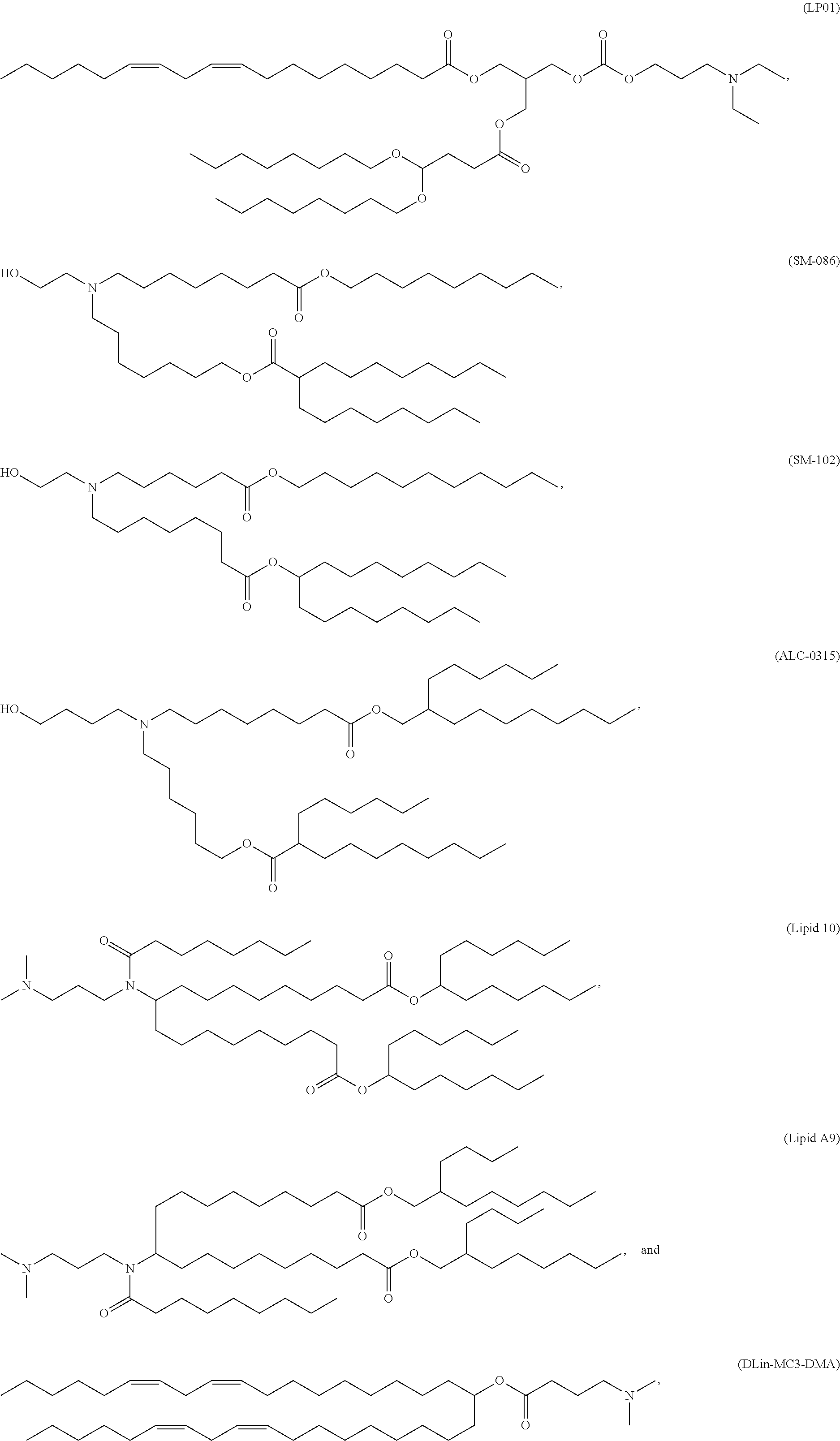

(see WO 2017/004143AL, which is incorporated herein by reference in its entirety).

In some embodiments, the lipid-based carrier (or lipid nanoformulation) further comprises one or more compounds described by WO 2021/113777 (e.g., a lipid of Formula (3) such as a lipid of Table 3 of WO 2021/113777), which is incorporated herein by reference in its entirety.

In one embodiment, the ionizable lipid is a lipid disclosed in Hou, X., et al. Nat Rev Mater 6, 1078-1094 (2021). https://doi.org/10.1038/s41578-021-00358-0 (e.g., L319, C12-200, and DLin-MC3-DMA), (which is incorporated by reference herein in its entirety).

Examples of other ionizable lipids that can be used in lipid-based carrier (or lipid nanoformulation) include, without limitation, one or more of the following formulas: X of US 2016/0311759; I of US 20150376115 or in US 2016/0376224; Compound 5 or Compound 6 in US 2016/0376224; I, IA, or II of U.S. Pat. No. 9,867,888; I, II or III of US 2016/0151284; I, IA, II, or IIA of US 2017/0210967; I-c of US 2015/0140070; A of US 2013/0178541; I of US 2013/0303587 or US 2013/0123338; I of US 2015/0141678; II, III, IV, or V of US 2015/0239926; I of US 2017/0119904; I or II of WO 2017/117528; A of US 2012/0149894; A of US 2015/0057373; A of WO 2013/116126; A of US 2013/0090372; A of US 2013/0274523; A of US 2013/0274504; A of US 2013/0053572; A of WO 2013/016058; A of WO 2012/162210; I of US 2008/042973; I, II, III, or IV of US 2012/01287670; I or II of US 2014/0200257; I, II, or III of US 2015/0203446; I or III of US 2015/0005363; I, IA, IB, IC, ID, II, IIA, IIB, IIC, IID, or III-XXIV of US 2014/0308304; of US 2013/0338210; I, II, III, or IV of WO 2009/132131; A of US 2012/01011478; I or XXXV of US 2012/0027796; XIV or XVII of US 2012/0058144; of US 2013/0323269; I of US 2011/0117125; I, II, or III of US 2011/0256175; I, II, III, IV, V, VI, VII, VIII, IX, X, XI, XII of US 2012/0202871; I, II, III, IV, V, VI, VII, VIII, X, XII, XIII, XIV, XV, or XVI of US 2011/0076335; I or II of US 2006/008378; I of WO2015/074085 (e.g., ATX-002); I of US 2013/0123338; I or X-A-Y-Z of US 2015/0064242; XVI, XVII, or XVIII of US 2013/0022649; I, II, or III of US 2013/0116307; I, II, or III of US 2013/0116307; I or II of US 2010/0062967; I-X of US 2013/0189351; I of US 2014/0039032; V of US 2018/0028664; I of US 2016/0317458; I of US 2013/0195920; 5, 6, or 10 of U.S. Pat. No. 10,221,127; 111-3 of WO 2018/081480; I-5 or 1-8 of WO 2020/081938; I of WO 2015/199952 (e.g., compound 6 or 22) and Table 1 therein; 18 or 25 of U.S. Pat. No. 9,867,888; A of US 2019/0136231; II of WO 2020/219876; 1 of US 2012/0027803; OF-02 of US 2019/0240349; 23 of U.S. Pat. No. 10,086,013; cKK-E12/A6 of Miao et al (2020); C12-200 of WO 2010/053572; 7 C1 of Dahlman et al (2017); 304-013 or 503-013 of Whitehead et al; TS-P4C2 of U.S. Pat. No. 9,708,628; I of WO 2020/106946; I of WO 2020/106946; (1), (2), (3), or (4) ofWO 2021/113777; and any one of Tables 1-16 of WO 2021/113777, all of which are incorporated herein by reference in their entirety.

In some embodiments, the lipid-based carrier (or lipid nanoformulation) further includes biodegradable ionizable lipids, for instance, (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate). See, e.g., lipids of WO 2019/067992, WO 2017/173054, WO 2015/095340, and WO 2014/136086, which are incorporated herein by reference in their entirety.

In some embodiments, the lipid particle comprises an ionizable lipid, a non-cationic lipid, a conjugated lipid that inhibits aggregation of particles, and a sterol. The amounts of these components can be varied independently and to achieve desired properties. For example, in some embodiments, the lipid nanoparticle comprises an ionizable lipid is in an amount from about 20 mol % to about 90 mol % of the total lipids (in other embodiments it may be 20-70% (mol), 30-60% (mol) or 40-50% (mol); about 50 mol % to about 90 mol % of the total lipid present in the lipid nanoparticle), a non-cationic lipid in an amount from about 5 mol % to about mol % of the total lipids, a conjugated lipid in an amount from about 0.5 mol % to about 20 mol % of the total lipids, and a sterol in an amount from about 20 mol % to about 50 mol % of the total lipids. The ratio of total lipid to nucleic acid can be varied as desired. For example, the total lipid to nucleic acid (mass or weight) ratio can be from about 10:1 to about 30:1.

In some embodiments, the lipid to nucleic acid ratio (mass/mass ratio; w/w ratio) can be in the range of from about 1:1 to about 25:1, from about 10:1 to about 14:1, from about 3:1 to about 15:1, from about 4:1 to about 10:1, from about 5:1 to about 9:1, or about 6:1 to about 9:1. The amounts of lipids and nucleic acid can be adjusted to provide a desired N/P ratio, for example, N/P ratio of 3, 4, 5, 6, 7, 8, 9, 10 or higher. Generally, the lipid nanoparticle formulation's overall lipid content can range from about 5 mg/ml to about 30 mg/mL.

Some non-limiting example of lipid compounds that may be used (e.g., in combination with other lipid components) to form lipid nanoparticles for the delivery of compositions described herein, e.g., nucleic acid (e.g., RNA) described herein includes,

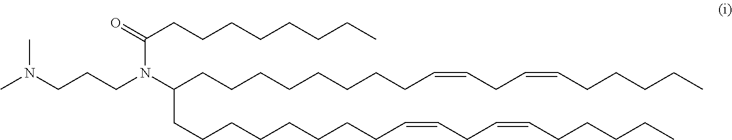 [SMILES: CCCCC/C=C\C/C=C\CCCCCCCCC(CCCCCCCC/C=C\C/C=C\CCCCC)N(CCCN(C)C)C(=O)CCCCCCCC]

In some embodiments an LNP comprising Formula (i) is used to deliver a TREM composition described herein to the liver and/or hepatocyte cells.

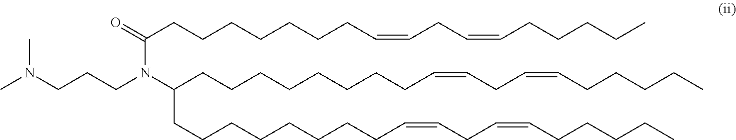 [SMILES: CCCCC/C=C\C/C=C\CCCCCCCCC(CCCCCCCC/C=C\C/C=C\CCCCC)N(CCCN(C)C)C(=O)CCCCCCC/C=C\C/C=C\CCCCC]

In some embodiments an LNP comprising Formula ii is use to deliver a TREM composition described herein to the liver and/or hepatocyte cells.

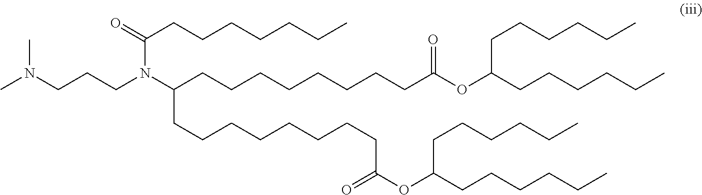 [SMILES: CCCCCCCC(=O)N(CCCN(C)C)C(CCCCCCCCC(=O)OC(CCCCCC)CCCCCC)CCCCCCCCC(=O)OC(CCCCCC)CCCCCC]

In some embodiments an LNP comprising Formula (iii) is used to deliver a TREM composition described herein to the liver and/or hepatocyte cells.

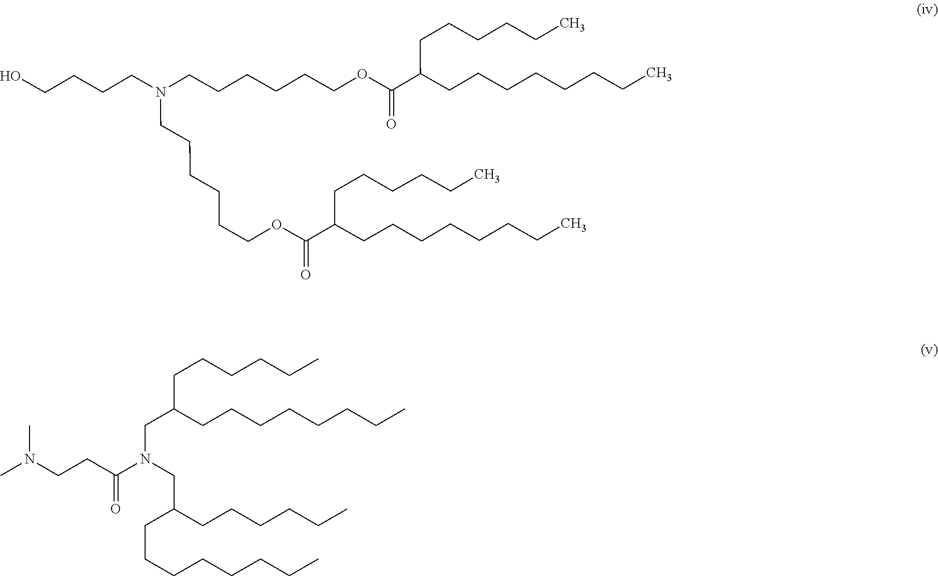

In some embodiments an LNP comprising Formula (v) is used to deliver a TREM composition described herein to the liver and/or hepatocyte cells.

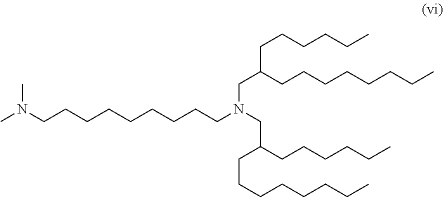 [SMILES: CCCCCCCCC(CCCCCC)CN(CCCCCCCCCN(C)C)CC(CCCCCC)CCCCCCCC]

In some embodiments an LNP comprising Formula (vi) is used to deliver a TREM composition described herein to the liver and/or hepatocyte cells.

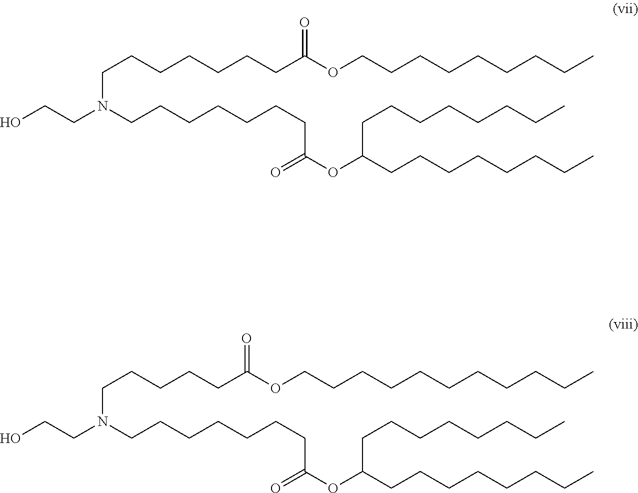 [SMILES: CCCCCCCCCOC(=O)CCCCCCCN(CCO)CCCCCCCC(=O)OC(CCCCCCCC)CCCCCCCC ; CCCCCCCCCCCOC(=O)CCCCCN(CCO)CCCCCCCC(=O)OC(CCCCCCCC)CCCCCCCC]

In some embodiments an LNP comprising Formula (viii) is used to deliver a TREM composition described herein to the liver and/or hepatocyte cells.

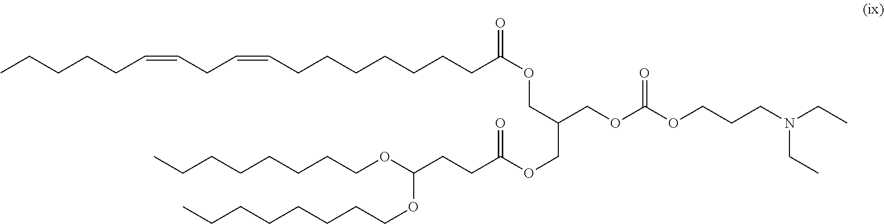 [SMILES: CCCCC/C=C\C/C=C\CCCCCCCC(=O)OCC(COC(=O)CCC(OCCCCCCCC)OCCCCCCCC)COC(=O)OCCCN(CC)CC]

In some embodiments an LNP comprising Formula (ix) is used to deliver a TREM composition described herein to the liver and/or hepatocyte cells.

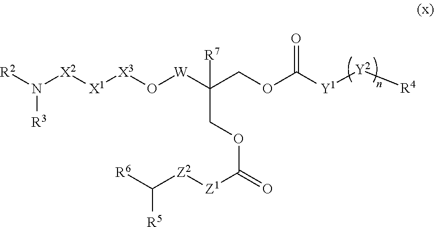

wherein X¹ is O, NR¹, or a direct bond, X² is C2-5 alkylene, X³ is C(═O) or a direct bond, R¹ is H or Me, R³ is Ci-3 alkyl, R² is Ci-3 alkyl, or R² taken together with the nitrogen atom to which it is attached and 1-3 carbon atoms of X² form a 4-, 5-, or 6-membered ring, or X¹ is NR¹, R¹ and R² taken together with the nitrogen atoms to which they are attached form a 5- or 6-membered ring, or R² taken together with R³ and the nitrogen atom to which they are attached form a 5-, 6-, or 7-membered ring, Y¹ is C2-12 alkylene, Y² is selected from

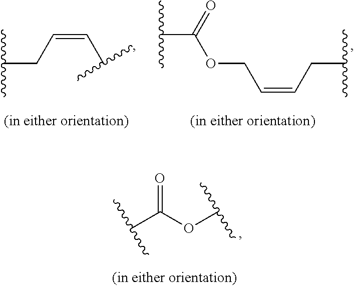 [SMILES: */C=C\C* ; *C/C=C\COC(*)=O ; *OC(*)=O]

n is 0 to 3, R₄ is Ci-15 alkyl, Z¹ is Ci-6 alkylene or a direct bond, Z⁷ is

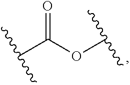 [SMILES: *OC(*)=O]

(in either orientation) or absent, provided that if Z is a direct bond, Z² is absent; R⁵ is C5-9 alky or 6 alkoxy, R⁶ is C5-9 alkyl or C6-10 alkoxy, W is methylene or a direct bond, and R⁷ is H or Me, or a salt thereof, provided that if R₃ and R² are C2 alkyls, X¹ is O, X² is linear C3 alkylene, X³ is C(═O), Y¹ is linear Ce alkylene, (Y²)n-R⁴ is:

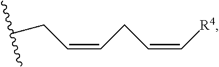

R⁴ is linear C alkyl, Z¹ is C2 alkylene, Z² is absent, W is methylene, and R⁷ is H, then R⁵ and R⁶ are not Cx alkoxy.

In some embodiments an LNP comprising Formula (xii) is used to deliver a TREM composition described herein to the liver and/or hepatocyte cells.

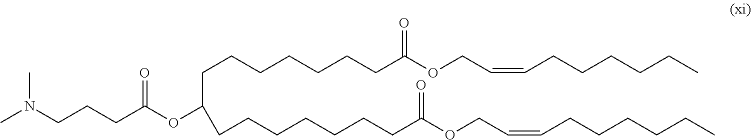 [SMILES: CCCCCC/C=C\COC(=O)CCCCCCCC(CCCCCCCC(=O)OC/C=C\CCCCCC)OC(=O)CCCN(C)C]

In some embodiments an LNP comprising Formula (xi) is used to deliver a TREM composition described herein to the liver and/or hepatocyte cells.

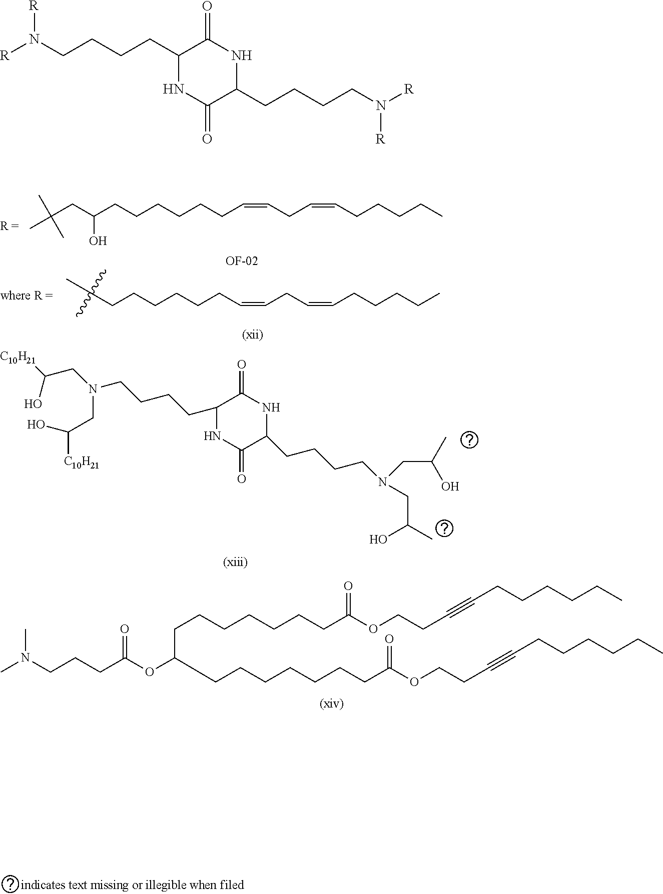

In some embodiments an LNP comprises a compound of Formula (xiii) and a compound of Formula (xiv).

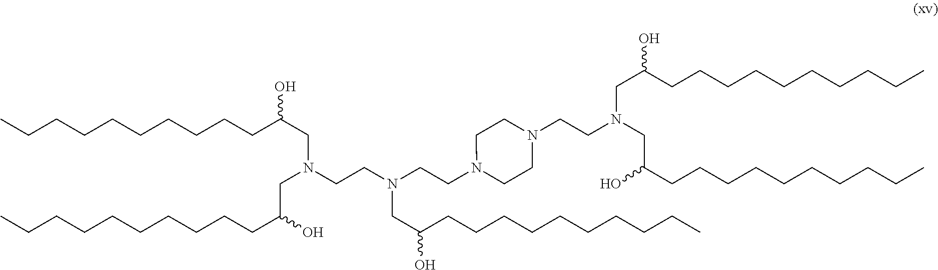 [SMILES: CCCCCCCCCCC(O)CN(CCN1CCN(CCN(CC(O)CCCCCCCCCC)CC(O)CCCCCCCCCC)CC1)CCN(CC(O)CCCCCCCCCC)CC(O)CCCCCCCCCC]

In some embodiments, an LNP comprising Formula (xv) is used to deliver a TREM composition described herein to the liver and/or hepatocyte cells.

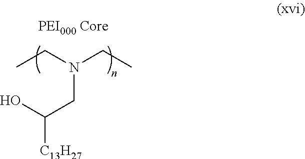

In some embodiments an LNP comprising a formulation of Formula (xvi) is used to deliver a TREM composition described herein to the lung endothelial cells.

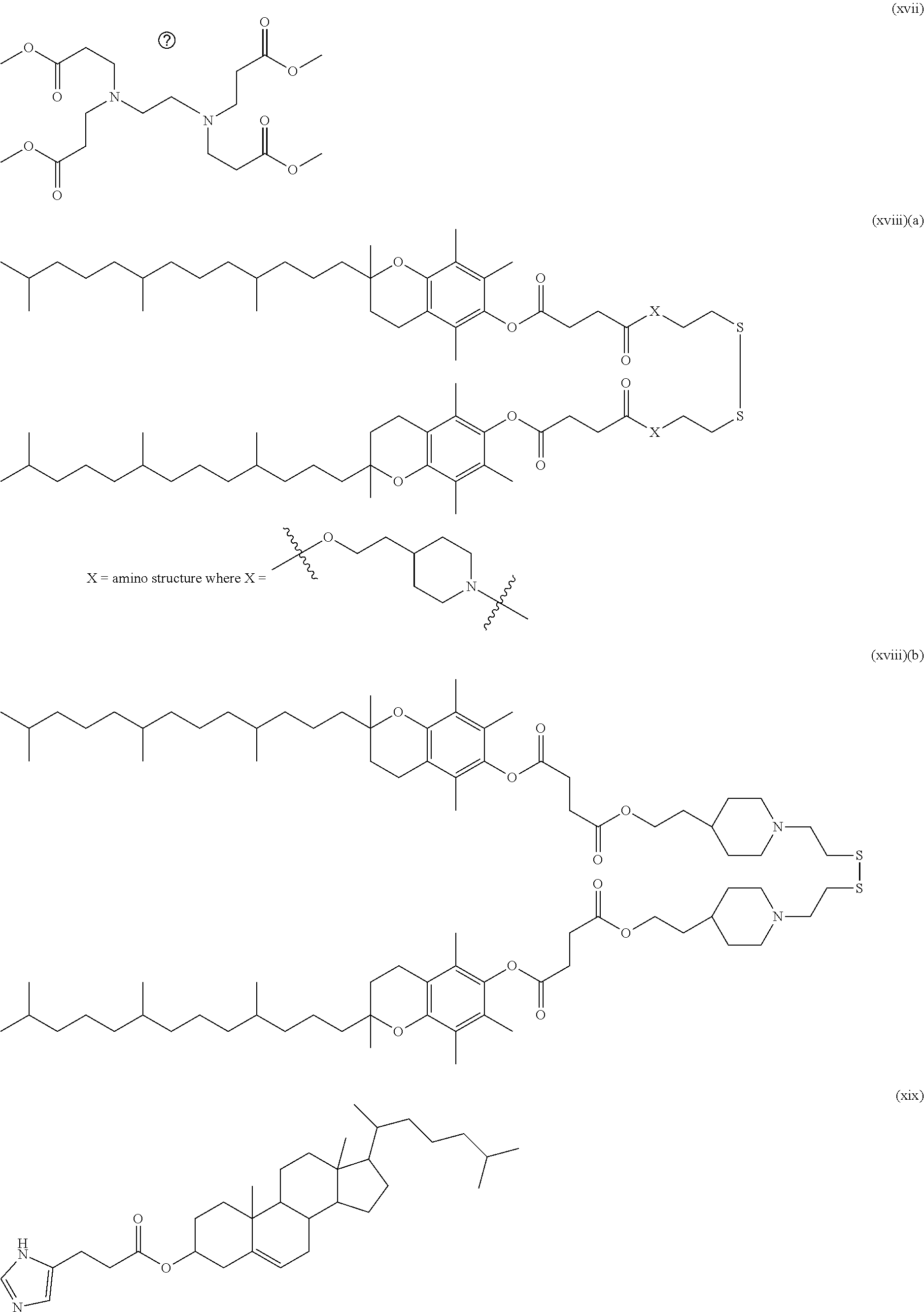

In some embodiments a lipid compound used to form lipid nanoparticles for the delivery of compositions described herein, e.g., a TREM described herein is made by one of the following reactions:

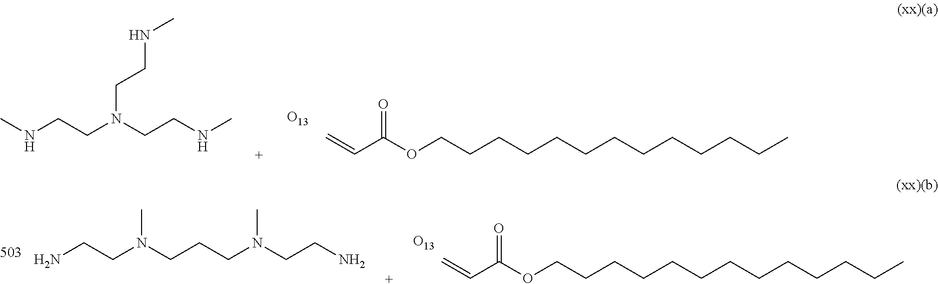

In some embodiments, a composition described herein (e.g., TREM composition) is provided in an LNP that comprises an ionizable lipid. In some embodiments, the ionizable lipid is heptadecan-9-yl 8-((2-hydroxyethyl)(6-oxo-6-(undecyloxy)hexyl)amino)octanoate (SM-102); e.g., as described in Example 1 of U.S. Pat. No. 9,867,888 (incorporated by reference herein in its entirety). In some embodiments, the ionizable lipid is 9Z,12Z)-3-((4,4-bis(octyloxy)butanoyl)oxy)-2-((((3-(diethylamino)propoxy)carbonyl)oxy)methyl)propyl octadeca-9,12-dienoate (LP01), e.g., as synthesized in Example 13 of WO2015/095340 (incorporated by reference herein in its entirety). In some embodiments, the ionizable lipid is Di((Z)-non-2-en-1-yl) 9-((4-dimethylamino)-butanoyl)oxy)heptadecanedioate (L319), e.g. as synthesized in Example 7, 8, or 9 of US2012/0027803 (incorporated by reference herein in its entirety). In some embodiments, the ionizable lipid is 1,1′-((2-(4-(2-((2-(Bis(2-hydroxydodecyl)amino)ethyl)(2-hydroxydodecyl) amino)ethyl)piperazin-1-yl)ethyl)azanediyl)bis(dodecan-2-ol) (C12-200), e.g., as synthesized in Examples 14 and 16 of WO2010/053572 (incorporated by reference herein in its entirety). In some embodiments, the ionizable lipid is Imidazole cholesterol ester (ICE) lipid (3S,10R, 13R, 17R)-10, 13-dimethyl-17-((R)-6-methylheptan-2-yl)-2, 3, 4, 7, 8, 9, 10, 11, 12, 13, 14, 15, 16, 17-tetradecahydro-1H-cyclopenta[a]phenanthren-3-yl 3-(1H-imidazol-4-yl)propanoate, e.g., Structure (I) from WO2020/106946 (incorporated by reference herein in its entirety).

In some embodiments, an ionizable lipid may be a cationic lipid, an ionizable cationic lipid, e.g., a cationic lipid that can ex ist in a positively charged or neutral form depending on pH, or an amine-containing lipid that can be readily protonated. In some embodiments, the cationic lipid is a lipid capable of being positively charged, e.g., under physiological conditions. Exemplary cationic lipids include one or more amine group(s) which bear the positive charge. In some embodiments, the lipid particle comprises a cationic lipid in formulation with one or more of neutral lipids, ionizable amine-containing lipids, biodegradable alkyne lipids, steroids, phospholipids including polyunsaturated lipids, structural lipids (e.g., sterols), PEG, cholesterol and polymer conjugated lipids. In some embodiments, the cationic lipid may be an ionizable cationic lipid. An exemplary cationic lipid as disclosed herein may have an effective pKa over 6.0. In embodiments, a lipid nanoparticle may comprise a second cationic lipid having a different effective pKa (e.g., greater than the first effective pKa), than the first cationic lipid. A lipid nanoparticle may comprise between 40 and 60 mol percent of a cationic lipid, a neutral lipid, a steroid, a polymer conjugated lipid, and a therapeutic agent, e.g., a TREM described herein, encapsulated within or associated with the lipid nanoparticle. In some embodiments, the TREM is co-formulated with the cationic lipid. The TREM may be adsorbed to the surface of an LNP, e.g., an LNP comprising a cationic lipid. In some embodiments, the TREM may be encapsulated in an LNP, e.g., an LNP comprising a cationic lipid. In some embodiments, the lipid nanoparticle may comprise a targeting moiety, e.g., coated with a targeting agent. In embodiments, the LNP formulation is biodegradable. In some embodiments, a lipid nanoparticle comprising one or more lipid described herein, e.g., Formula (i), (ii), (ii), (vii) and/or (ix) encapsulates at least 1%, at least 5%, at least 10%, at least 20%, at least 30%, at least 40%, at least 50%, at least 60%, at least 70%, at least 80%, at least 90%, at least 92%, at least 95%, at least 97%, at least 98% or 100% of a TREM.

Exemplary ionizable lipids that can be used in lipid nanoparticle formulations include, without limitation, those listed in Table 1 of WO2019051289, incorporated herein by reference. Additional exemplary lipids include, without limitation, one or more of the following formulae: X of US2016/0311759; I of US20150376115 or in US2016/0376224; I, II or III of US20160151284; I, IA, II, or IIA of US20170210967; I-c of US20150140070; A of US2013/0178541; I of US2013/0303587 or US2013/0123338; I of US2015/0141678; II, III, IV, or V of US2015/0239926; I of US2017/0119904; I or II of WO2017/117528; A of US2012/0149894; A of US2015/0057373; A of WO2013/116126; A of US2013/0090372; A of US2013/0274523; A of US2013/0274504; A of US2013/0053572; A of WO2013/016058; A of WO2012/162210; I of US2008/042973; I, II, III, or IV of US2012/01287670; I or II of US2014/0200257; I, II, or III of US2015/0203446; I or III of US2015/0005363; I, IA, IB, IC, ID, II, IIA, IIB, IIC, IID, or III-XXIV of US2014/0308304; of US2013/0338210; I, II, III, or IV of WO2009/132131; A of US2012/01011478; I or XXXV of US2012/0027796; XIV or XVII of US2012/0058144; of US2013/0323269; I of US2011/0117125; 1, II, or III of US2011/0256175; I, II, III, IV, V, VI, VII, VIII, IX, X, XI, XII of US2012/0202871; I, II, III, IV, V, VI, VII, VIII, X, XII, XIII, XIV, XV, or XVI of US2011/0076335; I or II of US2006/008378; I of US2013/0123338; I or X-A-Y-Z of US2015/0064242; XVI, XVII, or XVIII of US2013/0022649; I, II, or III of US2013/0116307; I, II, or III of US2013/0116307; I or II of US2010/0062967; I-X of US2013/0189351; I of US2014/0039032; V of US2018/0028664; I of US2016/0317458; I of US2013/0195920; 5, 6, or 10 of U.S. Pat. No. 10,221,127; III-3 of WO2018/081480; I-5 or I-8 of WO2020/081938; 18 or 25 of U.S. Pat. No. 9,867,888; A of US2019/0136231; II of WO2020/219876; 1 of US2012/0027803; OF-02 of US2019/0240349; 23 of U.S. Pat. No. 10,086,013; cKK-E12/A6 of Miao et al (2020); C12-200 of WO2010/053572; 7C1 of Dahlman et al (2017); 304-013 or 503-013 of Whitehead et al; TS-P4C2 of U.S. Pat. No. 9,708,628; I of WO2020/106946; I of WO2020/106946.

In some embodiments, the ionizable lipid is MC3 (6Z,9Z,28Z,3 IZ)-heptatriaconta-6,9,28,3 1-tetraen-19-yl-4-(dimethylamino) butanoate (DLin-MC3-DMA or MC3), e.g., as described in Example 9 of WO2019051289A9 (incorporated by reference herein in its entirety). In some embodiments, the ionizable lipid is the lipid ATX-002, e.g., as described in Example 10 of WO2019051289A9 (incorporated by reference herein in its entirety). In some embodiments, the ionizable lipid is (13Z,16Z)-A,A-dimethyl-3-nonyldocosa-13, 16-dien-1-amine (Compound 32), e.g., as described in Example 11 of WO2019051289A9 (incorporated by reference herein in its entirety). In some embodiments, the ionizable lipid is Compound 6 or Compound 22, e.g., as described in Example 12 of WO2019051289A9 (incorporated by reference herein in its entirety).

### Non-Cationic Lipids (e.g., Phospholipids)

In some embodiments, the lipid-based carrier (or lipid nanoformulation) further comprises one or more non-cationic lipids. In some embodiments, the non-cationic lipid is a phospholipid. In some embodiments, the non-cationic lipid is a phospholipid substitute or replacement. In some embodiments, the non-cationic lipid is a negatively charged (anionic) lipid.

Exemplary non-cationic lipids include, but are not limited to, distearoyl-sn-glycero-phosphoethanolamine, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), monomethyl-phosphatidylethanolamine (such as 16-O-monomethyl PE), dimethyl-phosphatidylethanolamine (such as 16-O-dimethyl PE), 18-1-trans PE, 1-stearoyl-2-oleoyl-phosphatidyethanolamine (SOPE), hydrogenated soy phosphatidylcholine (HSPC), egg phosphatidylcholine (EPC), dioleoylphosphatidylserine (DOPS), sphingomyelin (SM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), 1,2-dilauroyl-sn-glycero-3-phosphocholine (DLPC), Sodium 1,2-ditetradecanoyl-sn-glycero-3-phosphate (DMPA), phosphatidylcholine (lecithin), phosphatidylethanolamine, lysolecithin, lysophosphatidylethanolamine, phosphatidylserine, phosphatidylinositol, sphingomyelin, egg sphingomyelin (ESM), phosphatidylethanolamine (cephalin), cardiolipin, phosphatidic acid, cerebrosides, dicetylphosphate, lysophosphatidylcholine, dilinoleoylphosphatidylcholine, or mixtures thereof. It is understood that other diacylphosphatidylcholine and diacylphosphatidylethanolamine phospholipids can also be used. The acyl groups in these lipids are preferably acyl groups derived from fatty acids having C₁₀-C₂₄ carbon chains, e.g., lauroyl, myristoyl, paimitoyl, stearoyl, or oleoyl. Additional exemplary lipids, in certain embodiments, include, without limitation, those described in Kim et al. (2020) dx.doi.org/10.1021/acs.nanolett.Oc01386, which is incorporated herein by reference. Such lipids include, in some embodiments, plant lipids found to improve liver transfection with mRNA (e.g., DGTS).

In some embodiments, the lipid-based carrier (or lipid nanoformulation) may comprise a combination of distearoylphosphatidylcholine/cholesterol, dipalmitoylphosphatidylcholine/cholesterol, dimyrystoylphosphatidylcholine/cholesterol, 1,2-Dioleoyl-sn-glycero-3-phosphocholine (DOPC)/cholesterol, or egg sphingomyelin/cholesterol.

Other examples of suitable non-cationic lipids include, without limitation, nonphosphorous lipids such as, e.g., stearylamine, dodecylamine, hexadecylamine, acetyl palmitate, glycerol ricinoleate, hexadecyl stearate, isopropyl myristate, amphoteric acrylic polymers, triethanolamine-lauryl sulfate, alkyl-aryl sulfate polyethyloxylated fatty acid amides, dioctadecyl dimethyl ammonium bromide, ceramide, sphingomyelin, and the like. Other non-cationic lipids are described in WO 2017/099823 or US 2018/0028664, which are incorporated herein by reference in their entirety.

In one embodiment, the lipid-based carrier (or lipid nanoformulation) further comprises one or more non-cationic lipid that is oleic acid or a compound of Formula I, II, or IV of US 2018/0028664, which is incorporated herein by reference in its entirety.

The non-cationic lipid content can be, for example, 0-30% (mol) of the total lipid components present. In some embodiments, the non-cationic lipid content is 5-20% (mol) or 10-15% (mol) of the total lipid components present.

In some embodiments, the lipid-based carrier (or lipid nanoformulation) further comprises a neutral lipid, and the molar ratio of an ionizable lipid to a neutral lipid ranges from about 2:1 to about 8:1 (e.g., about 2:1, 3:1, 4:1, 5:1, 6:1, 7:1, or 8:1).

In some embodiments, the lipid-based carrier (or lipid nanoformulation) does not include any phospholipids.

In some embodiments, the lipid-based carrier (or lipid nanoformulation) can further include one or more phospholipids, and optionally one or more additional molecules of similar molecular shape and dimensions having both a hydrophobic moiety and a hydrophilic moiety (e.g., cholesterol).

Exemplary non-cationic lipids include, but are not limited to, distearoyl-sn-glycero-phosphoethanolamine, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), monomethyl-phosphatidylethanolamine (such as 16-O-monomethyl PE), dimethyl-phosphatidylethanolamine (such as 16-O-dimethyl PE), 18-1-trans PE, 1-stearoyl-2-oleoyl-phosphatidyethanolamine (SOPE), hydrogenated soy phosphatidylcholine (HSPC), egg phosphatidylcholine (EPC), dioleoylphosphatidylserine (DOPS), sphingomyelin (SM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidicacid, cerebrosides, dicetylphosphate, lysophosphatidylcholine, dilinoleoylphosphatidylcholine, or mixtures thereof. It is understood that other diacylphosphatidylcholine and diacylphosphatidylethanolamine phospholipids can also be used. The acyl groups in these lipids are preferably acyl groups derived from fatty acids having C10-C24 carbon chains, e.g., lauroyl, myristoyl, paimitoyl, stearoyl, or oleoyl. Additional exemplary lipids, in certain embodiments, include, without limitation, those described in Kim et al. (2020) dx.doi.org/10.1021/acs.nanolett.Oc01386, incorporated herein by reference. Such lipids include, in some embodiments, plant lipids found to improve liver transfection with mRNA (e.g., DGTS).

Other examples of non-cationic lipids suitable for use in the lipid nanoparticles include, without limitation, nonphosphorous lipids such as, e.g., stearylamine, dodeeylamine, hexadecylamine, acetyl palmitate, glycerol ricinoleate, hexadecyl stereate, isopropyl myristate, amphoteric acrylic polymers, triethanolamine-lauryl sulfate, alkyl-aryl sulfate polyethyloxylated fatty acid amides, dioctadecyl dimethyl ammonium bromide, ceramide, sphingomyelin, and the like. Other non-cationic lipids are described in WO2017/099823 or US patent publication US2018/0028664, the contents of which is incorporated herein by reference in their entirety.

In some embodiments, the non-cationic lipid is oleic acid or a compound of Formula I, II, or IV of US2018/0028664, incorporated herein by reference in its entirety. The non-cationic lipid can comprise, for example, 0-30% (mol) of the total lipid present in the lipid nanoparticle. In some embodiments, the non-cationic lipid content is 5-20% (mol) or 10-15% (mol) of the total lipid present in the lipid nanoparticle. In embodiments, the molar ratio of ionizable lipid to the neutral lipid ranges from about 2:1 to about 8:1 (e.g., about 2:1, 3:1, 4:1, 5:1, 6:1, 7:1, or 8:1).

In some embodiments, the lipid nanoparticles do not comprise any phospholipids.

### Structural Lipids

The lipid-based carrier (or lipid nanoformulation) described herein may further comprise one or more structural lipids. As used herein, the term “structural lipid” refers to sterols (e.g., cholesterol) and also to lipids containing sterol moieties.

Incorporation of structural lipids in the lipid nanoparticle may help mitigate aggregation of other lipid in the particle. Structural lipids can be selected from the group including but not limited to, cholesterol or cholesterol derivative, fecosterol, sitosterol, ergosterol, campesterol, stigmasterol, brassicasterol, tomatidine, tomatine, ursolic acid, alpha-tocopherol, hopanoids, phytosterols, steroids, and mixtures thereof. In some embodiments, the structural lipid is a sterol. In certain embodiments, the structural lipid is a steroid. In certain embodiments, the structural lipid is cholesterol. In certain embodiments, the structural lipid is an analog of cholesterol. In certain embodiments, the structural lipid is alpha-tocopherol.

In some embodiments, structural lipids may be incorporated into the lipid-based carrier at molar ratios ranging from about 0.1 to 1.0 (cholesterol phospholipid).

In some embodiments, sterols, when present, can include one or more of cholesterol or cholesterol derivatives, such as those described in WO 2009/127060 or US 2010/0130588, which are incorporated herein by reference in their entirety. Additional exemplary sterols include phytosterols, including those described in Eygeris et al. (2020), Nano Lett. 2020; 20(6):4543-4549, incorporated herein by reference.

In some embodiments, the structural lipid is a cholesterol derivative. Non-limiting examples of cholesterol derivatives include polar analogues such as 5a-cholestanol, 53-coprostanol, cholesteryl-(2′-hydroxy)-ethyl ether, cholesteryl-(4-hydroxy)-butyl ether, and 6-ketocholestanol; non-polar analogues such as 5a-cholestane, cholestenone, 5a-cholestanone, 5p-cholestanone, and cholesteryl decanoate; and mixtures thereof. In some embodiments, the cholesterol derivative is a polar analogue, e.g., cholesteryl-(4-hydroxy)-butyl ether. Exemplary cholesterol derivatives are described in WO 2009/127060 and US 2010/0130588, each of which is incorporated herein by reference in its entirety.

In some embodiments, the lipid-based carrier (or lipid nanoformulation) further comprises sterol in an amount of 0-50 mol % (e.g., 0-10 mol %, 10-20 mol %, 20-50 mol %, 20-mol %, 30-40 mol %, or 40-50 mol %) of the total lipid components.

In some aspects, the lipid nanoparticle can further comprise a component, such as a sterol, to provide membrane integrity. One exemplary sterol that can be used in the lipid nanoparticle is cholesterol and derivatives thereof. Non-limiting examples of cholesterol derivatives include polar analogues such as 5a-choiestanol, 53-coprostanol, choiesteryl-(2′-hydroxy)-ethyl ether, choiesteryl-(4′-hydroxy)-butyl ether, and 6-ketocholestanol; non-polar analogues such as 5a-cholestane, cholestenone, 5a-cholestanone, 5p-cholestanone, and cholesteryl decanoate; and mixtures thereof. In some embodiments, the cholesterol derivative is a polar analogue, e.g., choiesteryl-(4′-hydroxy)-buty 1 ether. Exemplary cholesterol derivatives are described in PCT publication WO2009/127060 and US patent publication US2010/0130588, each of which is incorporated herein by reference in its entirety.

In some embodiments, the component providing membrane integrity, such as a sterol, can comprise 0-50% (mol) (e.g., 0-10%, 10-20%, 20-30%, 30-40%, or 40-50%) of the total lipid present in the lipid nanoparticle. In some embodiments, such a component is 20-50% (mol) 30-40% (mol) of the total lipid content of the lipid nanoparticle.

### Polymers and Polyethylene Glycol (PEG)-Lipids

In some embodiments, the lipid-based carrier (or lipid nanoformulation) may include one or more polymers or co-polymers, e.g., poly(lactic-co-glycolic acid) (PFAG) nanoparticles.

In some embodiments, the lipid-based carrier (or lipid nanoformulation) may include one or more polyethylene glycol (PEG) lipid. Examples of useful PEG-lipids include, but are not limited to, 1,2-Diacyl-sn-Glycero-3-Phosphoethanolamine-N-[Methoxy(Polyethylene glycol)-350](mPEG 350 PE); 1,2-Diacyl-sn-Glycero-3-Phosphoethanolamine-N-[Methoxy(Polyethylene glycol)-550](mPEG 550 PE); 1,2-Diacyl-sn-Glycero-3-Phosphoethanolamine-N-[Methoxy(Polyethylene glycol)-750](mPEG 750 PE); 1,2-Diacyl-sn-Glycero-3-Phosphoethanolamine-N-[Methoxy(Polyethylene glycol)-1000](mPEG 1000 PE); 1,2-Diacyl-sn-Glycero-3-Phosphoethanolamine-N-[Methoxy(Polyethylene glycol)-2000](mPEG 2000 PE); 1,2-Diacyl-sn-Glycero-3-Phosphoethanolamine-N-[Methoxy(Polyethylene glycol)-3000](mPEG 3000 PE), 1,2-Diacyl-sn-Glycero-3-Phosphoethanolamine-N-[Methoxy(Polyethylene glycol)-5000](mPEG 5000 PE); N-Acyl-Sphingosine-1-[Succinyl(Methoxy Polyethylene Glycol) 750](mPEG 750 Ceramide); N-Acyl-Sphingosine-1-[Succinyl(Methoxy Polyethylene Glycol) 2000](mPEG 2000 Ceramide); and N-Acyl-Sphingosine-1-[Succinyl(Methoxy Polyethylene Glycol) 5000](mPEG 5000 Ceramide). In some embodiments, the PEG lipid is a polyethyleneglycol-diacylglycerol (i.e, polyethyleneglycol diacylglycerol (PEG-DAG), PEG-cholesterol, or PEG-DMB) conjugate.

In some embodiments, the lipid-based carrier (or nanoformulation) includes one or more conjugated lipids (such as PEG-conjugated lipids or lipids conjugated to polymers described in Table 5 of WO 2019/217941, which is incorporated herein by reference in its entirety). In some embodiments, the one or more conjugated lipids is formulated with one or more ionic lipids (e.g., non-cationic lipid such as a neutral or anionic, or zwitterionic lipid); and one or more sterols (e.g., cholesterol).

The PEG conjugate can comprise a PEG-dilaurylglycerol (C12), a PEG-dimyristoylglycerol (C14), a PEG-dipalmitoylglycerol (C16), a PEG-disterylglycerol (C18), PEG-dilaurylglycamide (C12), PEG-dimyristylglycamide (C14), PEG-dipalmitoylglycamide (C16), and PEG-disterylglycamide (C18).

In some embodiments, conjugated lipids, when present, can include one or more of PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-O-(2,3-di(tetradecanoyloxy)propyl-1-0-(w-methoxy(polyethoxy)ethyl) butanedioate (PEG-S-DMG)), PEG dialkoxypropylcarbam, N-(carbonyl-methoxypolyethylene glycol 2000)-1,2-distearoyl-sn-glycero-3-phosphoethanolamine sodium salt, and those described in Table 2 of WO 2019/051289 (which is herein incorporated by reference in its entirety), and combinations of the foregoing.

Additional exemplary PEG-lipid conjugates are described, for example, in U.S. Pat. Nos. 5,885,613, 6,287,591, US 2003/0077829, US 2003/0077829, US 2005/0175682, US 2008/0020058, US 2011/0117125, US 2010/0130588, US 2016/0376224, US 2017/0119904, US 2018/0028664, and WO 2017/099823, all of which are incorporated herein by reference in their entirety.

In some embodiments, the PEG-lipid is a compound of Formula III, III-a-I, III-a-2, III-b-1, III-b-2, or V of US 2018/0028664, which is incorporated herein by reference in its entirety. In some embodiments, the PEG-lipid is of Formula II of US 2015/0376115 or US 2016/0376224, both of which are incorporated herein by reference in their entirety. In some embodiments, the PEG-DAA conjugate can be, for example, PEG-dilauryloxypropyl, PEG-dimyristyloxypropyl, PEG-dipalmityloxypropyl, or PEG-distearyloxypropyl. In some embodiments, the PEG-lipid includes one of the following:

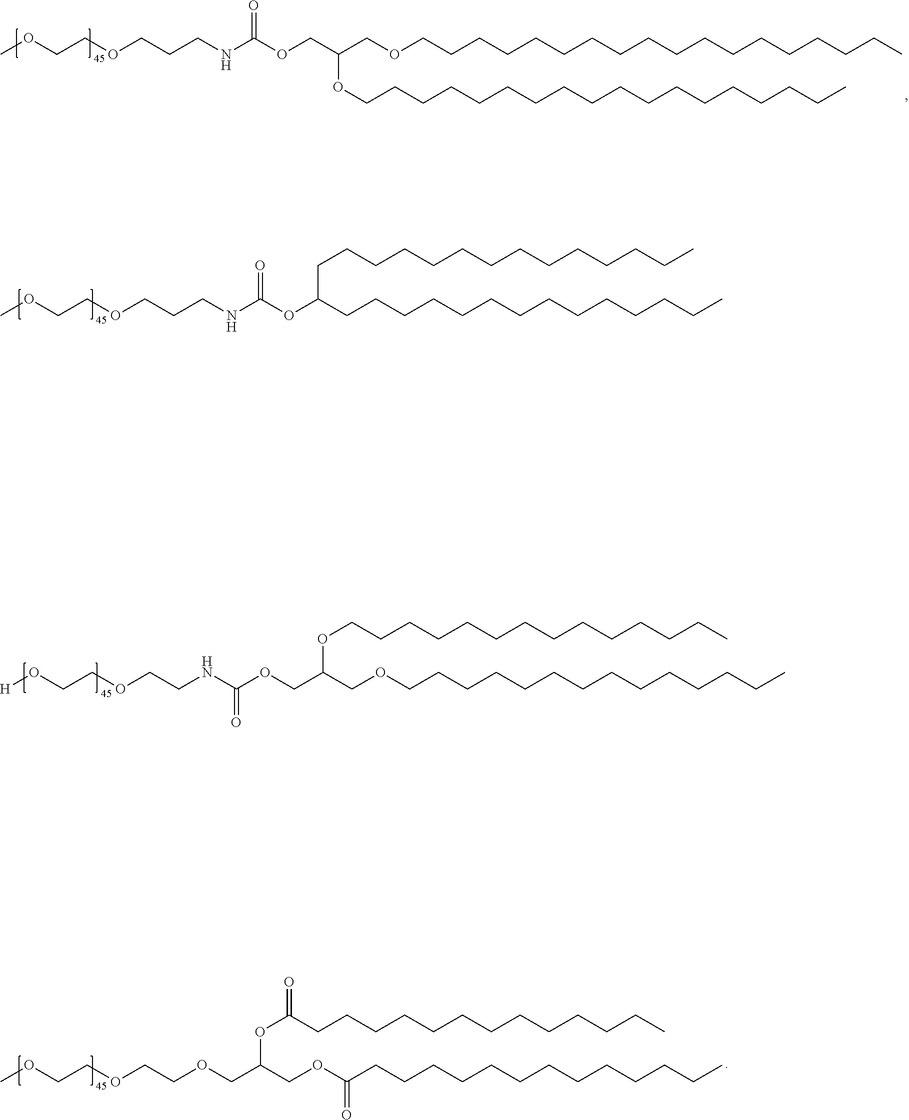

In some embodiments, lipids conjugated with a molecule other than a PEG can also be used in place of PEG-lipid. For example, polyoxazoline (POZ)-lipid conjugates, polyamide-lipid conjugates (such as ATTA-lipid conjugates), and cationic-polymer lipid (GPL) conjugates can be used in place of or in addition to the PEG-lipid.

Exemplary conjugated lipids, e.g., PEG-lipids, (POZ)-lipid conjugates, ATTA-lipid conjugates and cationic polymer-lipids, include those described in Table 2 of WO 2019/051289A9, which is incorporated herein by reference in its entirety.

In some embodiments, the conjugated lipid (e.g., the PEGylated lipid) can be present in an amount of 0-20 mol % of the total lipid components present in the lipid-based carrier (or lipid nanoformulation). In some embodiments, the conjugated lipid (e.g., the PEGylated lipid) content is 0.5-10 mol % or 2-5 mol % of the total lipid components.

When needed, the lipid-based carrier (or lipid nanoformulation) described herein may be coated with a polymer layer to enhance stability in vivo (e.g., sterically stabilized LNPs).

Examples of suitable polymers include, but are not limited to, poly(ethylene glycol), which may form a hydrophilic surface layer that improves the circulation half-life of liposomes and enhances the amount of lipid nanoformulations (e.g., liposomes or LNPs) that reach therapeutic targets. See, e.g., Working et al. J Pharmacol Exp Ther, 289: 1128-1133 (1999); Gabizon et al., J Controlled Release 53: 275-279 (1998); Adlakha Hutcheon et al., Nat Biotechnol 17: 775-779 (1999); and Koning et al., Biochim Biophys Acta 1420: 153-167 (1999), which are incorporated herein by reference in their entirety.

In some embodiments, the lipid nanoparticle can comprise a polyethylene glycol (PEG) or a conjugated lipid molecule. Generally, these are used to inhibit aggregation of lipid nanoparticles and/or provide steric stabilization. Exemplary conjugated lipids include, but are not limited to, PEG-lipid conjugates, polyoxazoline (POZ)-lipid conjugates, polyamide-lipid conjugates (such as ATTA-lipid conjugates), cationic-polymer lipid (CPL) conjugates, and mixtures thereof. In some embodiments, the conjugated lipid molecule is a PEG-lipid conjugate, for example, a (methoxy polyethylene glycol)-conjugated lipid.

Exemplary PEG-lipid conjugates include, but are not limited to, PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-0-(2′,3′-di(tetradecanoyloxy)propyl-1-0-(w-methoxy(polyethoxy)ethyl) butanedioate (PEG-S-DMG)), PEG dialkoxypropylcarbam, N-(carbonyl-methoxypolyethylene glycol 2000)-1,2-distearoyl-sn-glycero-3-phosphoethanolamine sodium salt, or a mixture thereof. Additional exemplary PEG-lipid conjugates are described, for example, in U.S. Pat. Nos. 5,885,613, 6,287,591, US2003/0077829, US2003/0077829, US2005/0175682, US2008/0020058, US2011/0117125, US2010/0130588, US2016/0376224, US2017/0119904, and US/099823, the contents of all of which are incorporated herein by reference in their entirety. In some embodiments, a PEG-lipid is a compound of Formula III, III-a-I, III-a-2, III-b-1, III-b-2, or V of US2018/0028664, the content of which is incorporated herein by reference in its entirety. In some embodiments, a PEG-lipid is of Formula II of US20150376115 or US2016/0376224, the content of both of which is incorporated herein by reference in its entirety. In some embodiments, the PEG-DAA conjugate can be, for example, PEG-dilauryloxypropyl, PEG-dimyristyloxypropyl, PEG-dipalmityloxypropyl, or PEG-distearyloxypropyl. The PEG-lipid can be one or more of PEG-DMG, PEG-dilaurylglycerol, PEG-dipalmitoylglycerol, PEG-disterylglycerol, PEG-dilaurylglycamide, PEG-dimyristylglycamide, PEG-dipalmitoylglycamide, PEG-disterylglycamide, PEG-cholesterol (1-[8′-(Cholest-5-en-3[beta]-oxy)carboxamido-3′,6′-dioxaoctanyl]carbamoyl-[omega]-methyl-poly(ethylene glycol), PEG-DMB (3,4-Ditetradecoxylbenzyl-[omega]-methyl-poly(ethylene glycol) ether), and 1,2-dimyristoyl-sn-glycero-3-phosphoethanolamine-N-[methoxy(polyethylene glycol)-2000]. In some embodiments, the PEG-lipid comprises PEG-DMG, 1,2-dimyristoyl-sn-glycero-3-phosphoethanolamine-N-[methoxy(polyethylene glycol)-2000]. In some embodiments, the PEG-lipid comprises a structure selected from:

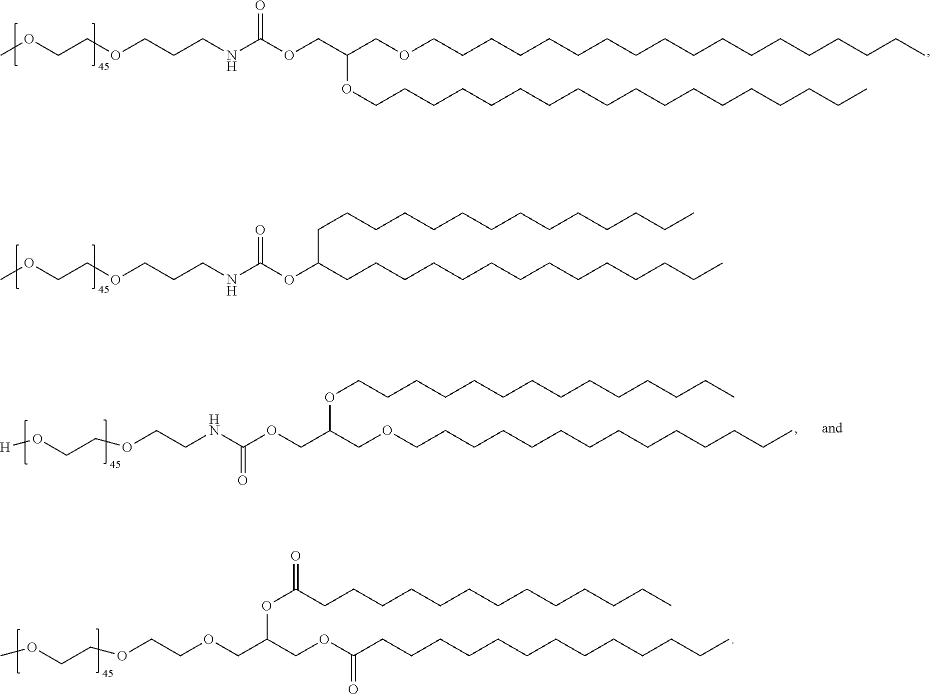

In some embodiments, lipids conjugated with a molecule other than a PEG can also be used in place of PEG-lipid. For example, polyoxazoline (POZ)-lipid conjugates, polyamide-lipid conjugates (such as ATTA-lipid conjugates), and cationic-polymer lipid (GPL) conjugates can be used in place of or in addition to the PEG-lipid.

Exemplary conjugated lipids, i.e., PEG-lipids, (POZ)-lipid conjugates, ATTA-lipid conjugates and cationic polymer-lipids are described in the PCT and LIS patent applications listed in Table 2 of WO2019051289A9, the contents of all of which are incorporated herein by reference in their entirety.

In some embodiments, the PEG or the conjugated lipid can comprise 0-20% (mol) of the total lipid present in the lipid nanoparticle. In some embodiments, PEG or the conjugated lipid content is 0.5-10% or 2-5% (mol) of the total lipid present in the lipid nanoparticle. Molar ratios of the ionizable lipid, non-cationic-lipid, sterol, and PEG/conjugated lipid can be varied as needed. For example, the lipid particle can comprise 30-70% ionizable lipid by mole or by total weight of the composition, 0-60% cholesterol by mole or by total weight of the composition, 0-30% non-cationic-lipid by mole or by total weight of the composition and 1-10% conjugated lipid by mole or by total weight of the composition. Preferably, the composition comprises 30-40% ionizable lipid by mole or by total weight of the composition, 40-50% cholesterol by mole or by total weight of the composition, and 10-20% non-cationic-lipid by mole or by total weight of the composition. In some other embodiments, the composition is 50-75% ionizable lipid by mole or by total weight of the composition, 20-40% cholesterol by mole or by total weight of the composition, and 5 to 10% non-cationic-lipid, by mole or by total weight of the composition and 1-10% conjugated lipid by mole or by total weight of the composition. The composition may contain 60-70% ionizable lipid by mole or by total weight of the composition, 25-35% cholesterol by mole or by total weight of the composition, and 5-10% non-cationic-lipid by mole or by total weight of the composition. The composition may also contain up to 90% ionizable lipid by mole or by total weight of the composition and 2 to 15% non-cationic lipid by mole or by total weight of the composition. The formulation may also be a lipid nanoparticle formulation, for example comprising 8-30% ionizable lipid by mole or by total weight of the composition, 5-30% non-cationic lipid by mole or by total weight of the composition, and 0-20% cholesterol by mole or by total weight of the composition; 4-25% ionizable lipid by mole or by total weight of the composition, 4-25% non-cationic lipid by mole or by total weight of the composition, 2 to 25% cholesterol by mole or by total weight of the composition, 10 to 35% conjugate lipid by mole or by total weight of the composition, and 5% cholesterol by mole or by total weight of the composition; or 2-30% ionizable lipid by mole or by total weight of the composition, 2-30% non-cationic lipid by mole or by total weight of the composition, 1 to 15% cholesterol by mole or by total weight of the composition, 2 to 35% conjugate lipid by mole or by total weight of the composition, and 1-20% cholesterol by mole or by total weight of the composition; or even up to 90% ionizable lipid by mole or by total weight of the composition and 2-10% non-cationic lipids by mole or by total weight of the composition, or even 100% cationic lipid by mole or by total weight of the composition. In some embodiments, the lipid particle formulation comprises ionizable lipid, phospholipid, cholesterol and a PEG-ylated lipid in a molar ratio of 50:10:38.5:1.5. In some other embodiments, the lipid particle formulation comprises ionizable lipid, cholesterol and a PEG-ylated lipid in a molar ratio of 60:38.5:1.5.

In some embodiments, the lipid particle comprises ionizable lipid, non-cationic lipid (e.g. phospholipid), a sterol (e.g., cholesterol) and a PEG-ylated lipid, where the molar ratio of lipids ranges from 20 to 70 mole percent for the ionizable lipid, with a target of 40-60, the mole percent of non-cationic lipid ranges from 0 to 30, with a target of 0 to 15, the mole percent of sterol ranges from 20 to 70, with a target of 30 to 50, and the mole percent of PEG-ylated lipid ranges from 1 to 6, with a target of 2 to 5.

In some embodiments, the lipid particle comprises ionizable lipid/non-cationic-lipid/sterol/conjugated lipid at a molar ratio of 50:10:38.5:1.5.

In an aspect, the disclosure provides a lipid nanoparticle formulation comprising phospholipids, lecithin, phosphatidylcholine and phosphatidylethanolamine.

In some embodiments, one or more additional compounds can also be included. Those compounds can be administered separately, or the additional compounds can be included in the lipid nanoparticles of the invention. In other words, the lipid nanoparticles can contain other compounds in addition to the nucleic acid or at least a second nucleic acid, different than the first. Without limitations, other additional compounds can be selected from the group consisting of small or large organic or inorganic molecules, monosaccharides, disaccharides, trisaccharides, oligosaccharides, polysaccharides, peptides, proteins, peptide analogs and derivatives thereof, peptidomimetics, nucleic acids, nucleic acid analogs and derivatives, an extract made from biological materials, or any combinations thereof.

In some embodiments, LNPs are directed to specific tissues by the addition of targeting domains. For example, biological ligands may be displayed on the surface of LNPs to enhance interaction with cells displaying cognate receptors, thus driving association with and cargo delivery to tissues wherein cells express the receptor. In some embodiments, the biological ligand may be a ligand that drives delivery to the liver, e.g., LNPs that display GalNAc result in delivery of nucleic acid cargo to hepatocytes that display asialoglycoprotein receptor (ASGPR).

The work of Akinc et al. Mol Ther 18(7):1357-1364 (2010) teaches the conjugation of a trivalent GalNAc ligand to a PEG-lipid (GalNAc-PEG-DSG) to yield LNPs dependent on ASGPR for observable LNP cargo effect (see, e.g., FIG. 6 of Akinc et al. 2010, supra). Other ligand-displaying LNP formulations, e.g., incorporating folate, transferrin, or antibodies, are discussed in WO2017223135, which is incorporated herein by reference in its entirety, in addition to the references used therein, namely Kolhatkar et al., Curr Drug Discov Technol. 2011 8:197-206; Musacchio and Torchilin, Front Biosci. 2011 16:1388-1412; Yu et al., Mol Membr Biol. 2010 27:286-298; Patil et al., Crit Rev Ther Drug Carrier Syst. 2008 25:1-61; Benoit et al., Biomacromolecules. 2011 12:2708-2714; Zhao et al., Expert Opin Drug Deliv. 2008 5:309-319; Akinc et al., Mol Ther. 2010 18:1357-1364; Srinivasan et al., Methods Mol Biol. 2012 820:105-116; Ben-Arie et al., Methods Mol Biol. 2012 757:497-507; Peer 2010 J Control Release. 20:63-68; Peer et al., Proc Natl Acad Sci USA. 2007 104:4095-4100; Kim et al., Methods Mol Biol. 2011 721:339-353; Subramanya et al., Mol Ther. 2010 18:2028-2037; Song et al., Nat Biotechnol. 2005 23:709-717; Peer et al., Science. 2008 319:627-630; and Peer and Lieberman, Gene Ther. 2011 18:1127-1133.

In some embodiments, LNPs are selected for tissue-specific activity by the addition of a Selective ORgan Targeting (SORT) molecule to a formulation comprising traditional components, such as ionizable cationic lipids, amphipathic phospholipids, cholesterol and poly(ethylene glycol) (PEG) lipids. The teachings of Cheng et al. Nat Nanotechnol 15(4):313-320 (2020) demonstrate that the addition of a supplemental “SORT” component precisely alters the in vivo RNA delivery profile and mediates tissue-specific (e.g., lungs, liver, spleen) gene delivery and editing as a function of the percentage and biophysical property of the SORT molecule.

In some embodiments, the LNPs comprise biodegradable, ionizable lipids. In some embodiments, the LNPs comprise (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate) or another ionizable lipid. See, e.g, lipids of WO2019/067992, WO/2017/173054, WO2015/095340, and WO2014/136086, as well as references provided therein. In some embodiments, the term cationic and ionizable in the context of LNP lipids is interchangeable, e.g., wherein ionizable lipids are cationic depending on the pH.

In some embodiments, the average LNP diameter of the LNP formulation may be between 10 s of nm and 100 s of nm, e.g., measured by dynamic light scattering (DLS). In some embodiments, the average LNP diameter of the LNP formulation may be from about 40 nm to about 150 nm, such as about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In some embodiments, the average LNP diameter of the LNP formulation may be from about 50 nm to about 100 nm, from about 50 nm to about 90 nm, from about 50 nm to about 80 nm, from about 50 nm to about 70 nm, from about 50 nm to about 60 nm, from about 60 nm to about 100 nm, from about 60 nm to about 90 nm, from about 60 nm to about 80 nm, from about 60 nm to about 70 nm, from about 70 nm to about 100 nm, from about 70 nm to about 90 nm, from about 70 nm to about 80 nm, from about 80 nm to about 100 nm, from about 80 nm to about 90 nm, or from about 90 nm to about 100 nm. In some embodiments, the average LNP diameter of the LNP formulation may be from about 70 nm to about 100 nm. In a particular embodiment, the average LNP diameter of the LNP formulation may be about 80 nm. In some embodiments, the average LNP diameter of the LNP formulation may be about 100 nm. In some embodiments, the average LNP diameter of the LNP formulation ranges from about 1 mm to about 500 mm, from about 5 mm to about 200 mm, from about 10 mm to about 100 mm, from about 20 mm to about 80 mm, from about 25 mm to about 60 mm, from about 30 mm to about 55 mm, from about 35 mm to about 50 mm, or from about 38 mm to about 42 mm.

A LNP may, in some instances, be relatively homogenous. A polydispersity index may be used to indicate the homogeneity of a LNP, e.g., the particle size distribution of the lipid nanoparticles. A small (e.g., less than 0.3) polydispersity index generally indicates a narrow particle size distribution. A LNP may have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a LNP may be from about 0.10 to about 0.20.

The zeta potential of a LNP may be used to indicate the electrokinetic potential of the composition. In some embodiments, the zeta potential may describe the surface charge of an LNP. Lipid nanoparticles with relatively low charges, positive or negative, are generally desirable, as more highly charged species may interact undesirably with cells, tissues, and other elements in the body. In some embodiments, the zeta potential of a LNP may be from about −10 mV to about +20 mV, from about −10 mV to about +15 mV, from about −10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

The efficiency of encapsulation of a TREM describes the amount of TREM that is encapsulated or otherwise associated with a LNP after preparation, relative to the initial amount provided. The encapsulation efficiency is desirably high (e.g., close to 100%). The encapsulation efficiency may be measured, for example, by comparing the amount of TREM in a solution containing the lipid nanoparticle before and after breaking up the lipid nanoparticle with one or more organic solvents or detergents. An anion exchange resin may be used to measure the amount of free protein or nucleic acid (e.g., RNA) in a solution. Fluorescence may be used to measure the amount of free TREM in a solution. For the lipid nanoparticles described herein, the encapsulation efficiency of a TREM may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 80%. In some embodiments, the encapsulation efficiency may be at least 90%. In some embodiments, the encapsulation efficiency may be at least 95%.

A LNP may optionally comprise one or more coatings. In some embodiments, a LNP may be formulated in a capsule, film, or table having a coating. A capsule, film, or tablet including a composition described herein may have any useful size, tensile strength, hardness or density.

Additional exemplary lipids, formulations, methods, and characterization of LNPs are taught by WO2020061457, which is incorporated herein by reference in its entirety.

In some embodiments, in vitro or ex vivo cell lipofections are performed using Lipofectamine MessengerMax (Thermo Fisher) or TransIT-mRNA Transfection Reagent (Mirus Bio). In certain embodiments, LNPs are formulated using the GenVoy_ILM ionizable lipid mix (Precision NanoSystems). In certain embodiments, LNPs are formulated using 2,2-dilinoleyl-4-dimethylaminoethyl-[1,3]-dioxolane (DLin-KC2-DMA) or dilinoleylmethyl-4-dimethylaminobutyrate (DLin-MC3-DMA or MC3), the formulation and in vivo use of which are taught in Jayaraman et al. Angew Chem Int Ed Engl 51(34):8529-8533 (2012), incorporated herein by reference in its entirety.

LNP formulations optimized for the delivery of CRISPR-Cas systems, e.g., Cas9-gRNA RNP, gRNA, Cas9 mRNA, are described in WO2019067992 and WO2019067910, both incorporated by reference.

Additional specific LNP formulations useful for delivery of nucleic acids are described in U.S. Pat. Nos. 8,158,601 and 8,168,775, both incorporated by reference, which include formulations used in patisiran, sold under the name ONPATTRO.

Exosomes can also be used as drug delivery vehicles for the TREM, TREM core fragment, TREM fragment, or TREM compositions or pharmaceutical TREM composition described herein. For a review, see Ha et al. July 2016. Acta Pharmaceutica Sinica B. Volume 6, Issue 4, Pages 287-296; https://doi.org/10.1016/j.apsb.2016.02.001.

Ex vivo differentiated red blood cells can also be used as a carrier for a TREM, TREM core fragment, TREM fragment, or TREM composition, or pharmaceutical TREM composition described herein. See, e.g., WO2015073587; WO2017123646; WO2017123644; WO2018102740; w02016183482; WO2015153102; WO2018151829; WO2018009838; Shi et al. 2014. Proc Natl Acad Sci USA. 111(28): 10131-10136; U.S. Pat. No. 9,644,180; Huang et al. 2017. Nature Communications 8: 423; Shi et al. 2014. Proc Natl Acad Sci USA. 111(28): 10131-10136.

Fusosome compositions, e.g., as described in WO2018208728, can also be used as carriers to deliver the TREM, TREM core fragment, TREM fragment, or TREM composition, or pharmaceutical TREM composition described herein.

Virosomes and virus-like particles (VLPs) can also be used as carriers to deliver a TREM, TREM core fragment, TREM fragment, or TREM composition, or pharmaceutical TREM composition described herein to targeted cells.

Plant nanovesicles, e.g., as described in WO2011097480A1, WO2013070324A1, or WO2017004526A1 can also be used as carriers to deliver the TREM, TREM core fragment, TREM fragment, or TREM composition, or pharmaceutical TREM composition described herein.

### Percentages of Lipid Nanoformulation Components

In some embodiments, the lipid-based carrier (or lipid nanoformulation) comprises one of more of the compounds described herein, optionally a non-cationic lipid (e.g., a phospholipid), a sterol, a neutral lipid, and optionally conjugated lipid (e.g., a PEGylated lipid) that inhibits aggregation of particles. In some embodiments, the lipid-based carrier (or lipid nanoformulation) further comprises a TREM (e.g., a TREM described herein). The amounts of these components can be varied independently and to achieve desired properties. For example, in some embodiments, the ionizable lipid including the lipid compounds described herein is present in an amount from about 20 mol % to about 100 mol % (e.g., 20-90 mol %, 20-80 mol %, 20-70 mol %, 25-100 mol %, 30-70 mol %, 30-60 mol %, 30-40 mol %, 40-50 mol %, or 50-90 mol %) of the total lipid components; a non-cationic lipid (e.g., phospholipid) is present in an amount from about 0 mol % to about 50 mol % (e.g., 0-40 mol %, 0-30 mol %, 5-50 mol %, 5-40 mol %, 5-30 mol %, or 5-10 mol %) of the total lipid components, a conjugated lipid (e.g., a PEGylated lipid) in an amount from about 0.5 mol % to about 20 mol % (e.g., 1-10 mol % or 5-10%) of the total lipid components, and a sterol in an amount from about 0 mol % to about 60 mol % (e.g., 0-50 mol %, 10-60 mol %, 10-50 mol %, 15-60 mol %, 15-50 mol %, 20-50 mol %, 20-mol %) of the total lipid components, provided that the total mol % of the lipid component does not exceed 100%.

In some embodiments, the lipid-based carrier (or lipid nanoformulation) comprises about 25-100 mol % of the ionizable lipid including the lipid compounds described herein, about 0-50 mol % phospholipid, about 0-50 mol % sterol, and about 0-10 mol % PEGylated lipid.

In some embodiments, the lipid-based carrier comprises a TREM (e.g., a TREM described herein) that is formulated in a lipid nanoparticle, wherein the lipid nanoparticle comprises about 25-100 mol % of the ionizable lipid including the lipid compounds described herein, about 0-50 mol % phospholipid, about 0-50 mol % sterol, and about 0-10 mol % PEGylated lipid. In some embodiments, the encapsulation efficiency of the payload may be at least 70%.

In one embodiment, the lipid-based carrier (or lipid nanoformulation) comprises about 25-100 mol % of the ionizable lipid including the lipid compounds described herein; about 0-40 mol % phospholipid (e.g., DSPC), about 0-50 mol % sterol (e.g., cholesterol), and about 0-10 mol % PEGylated lipid.

In some embodiments, the lipid-based carrier comprises a TREM (e.g., a TREM described herein) that is formulated in a lipid nanoparticle, wherein the lipid nanoparticle comprises about 25-100 mol % of the ionizable lipid including the lipid compounds described herein; about 0-40 mol % phospholipid (e.g., DSPC), about 0-50 mol % sterol (e.g., cholesterol), and about 0-10 mol % PEGylated lipid. In some embodiments, the encapsulation efficiency of the payload may be at least 70%.

In some embodiments, the lipid-based carrier (or lipid nanoformulation) comprises about 30-60 mol % (e.g., about 35-55 mol %, or about 40-50 mol %) of the ionizable lipid including the lipid compounds described herein, about 0-30 mol % (e.g., 5-25 mol %, or 10-20 mol %) phospholipid, about 15-50 mol % (e.g., 18.5-48.5 mol %, or 30-40 mol %) sterol, and about 0-10 mol % (e.g., 1-5 mol %, or 1.5-2.5 mol %) PEGylated lipid.

In some embodiments, the lipid-based carrier comprises a TREM (e.g., a TREM described herein) that is formulated in a lipid nanoparticle, wherein the lipid nanoparticle comprises about 30-60 mol % (e.g., about 35-55 mol %, or about 40-50 mol %) of the ionizable lipid including the lipid compounds described herein, about 0-30 mol % (e.g., 5-25 mol %, or 10-mol %) phospholipid, about 15-50 mol % (e.g., 18.5-48.5 mol %, or 30-40 mol %) sterol, and about 0-10 mol % (e.g., 1-5 mol %, or 1.5-2.5 mol %) PEGylated lipid. In some embodiments, the encapsulation efficiency of the payload may be at least 70%.

In some embodiments, molar ratios of ionizable lipid/sterol/phospholipid (or another structural lipid)/PEG-lipid/additional components is varied in the following ranges: ionizable lipid (25-100%); phospholipid (DSPC) (0-40%); sterol (0-50%); and PEG lipid (0-5%).

In some embodiments, the lipid-based carrier comprises a TREM (e.g., a TREM described herein) that is formulated in a lipid nanoparticle, wherein the lipid nanoparticle comprises molar ratios of ionizable lipid/sterol/phospholipid (or another structural lipid)/PEG-lipid/additional components in the following ranges: ionizable lipid (25-100%); phospholipid (DSPC) (0-40%); sterol (0-50%); and PEG lipid (0-5%). In some embodiments, the encapsulation efficiency of the payload may be at least 70%.

In some embodiments, the lipid-based carrier (or lipid nanoformulation) comprises, by mol % or wt % of the total lipid components, 50-75% ionizable lipid (including the lipid compound as described herein), 20-40% sterol (e.g., cholesterol or derivative), 0 to 10% non-cationic-lipid, and 1-10% conjugated lipid (e.g., the PEGylated lipid).

In some embodiments, the lipid-based carrier comprises a TREM (e.g., a TREM described herein) that is formulated in a lipid nanoparticle, wherein the lipid nanoparticle comprises, by mol % or wt % of the total lipid components, 50-75% ionizable lipid (including the lipid compound as described herein), 20-40% sterol (e.g., cholesterol or derivative), 0 to 10% non-cationic-lipid, and 1-10% conjugated lipid (e.g., the PEGylated lipid). In some embodiments, the encapsulation efficiency of the payload may be at least 70%.

In some embodiments, the lipid-based carrier (or lipid nanoformulation) comprises (i) a TREM (e.g., a TREM described herein); (ii) a cationic lipid comprising from 50 mol % to 65 mol % of the total lipid present in the lipid-based carrier; (iii) a non-cationic lipid comprising a mixture of a phospholipid and a cholesterol derivative thereof, wherein the phospholipid comprises from 3 mol % to 15 mol % of the total lipid present in the lipid-based carrier and the cholesterol or derivative thereof comprises from 30 mol % to 40 mol % of the total lipid present in the lipid-based carrier; and (iv) a conjugated lipid comprising 0.5 mol % to 2 mol % of the total lipid present in the particle.

In some embodiments, the lipid-based carrier (or lipid nanoformulation) comprises (i) a TREM (e.g., a TREM described herein); (ii) a cationic lipid comprising from 50 mol % to 85 mol % of the total lipid present in the lipid-based carrier; (iii) a non-cationic lipid comprising from 13 mol % to 49.5 mol % of the total lipid present in the lipid-based carrier; and (d) a conjugated lipid comprising from 0.5 mol % to 2 mol % of the total lipid present in the lipid-based carrier.

In some embodiments, the phospholipid component in the mixture may be present from 2 mol % to 20 mol %, from 2 mol % to 15 mol %, from 2 mol % to 12 mol %, from 4 mol % to 15 mol %, from 4 mol % to 10 mol %, from 5 mol % to 10 mol %, (or any fraction of these ranges) of the total lipid components. In some embodiments, the lipid-based carrier (or lipid nanoformulation) is phospholipid-free.

In some embodiments, the sterol component (e.g. cholesterol or derivative) in the mixture may comprise from 25 mol % to 45 mol %, from 25 mol % to 40 mol %, from 25 mol % to 35 mol %, from 25 mol % to 30 mol %, from 30 mol % to 45 mol %, from 30 mol % to 40 mol %, from mol % to 35 mol %, from 35 mol % to 40 mol %, from 27 mol % to 37 mol %, or from 27 mol % to 35 mol % (or any fraction of these ranges) of the total lipid components.

In some embodiments, the non-ionizable lipid components in the lipid-based carrier (or lipid nanoformulation) may be present from 5 mol % to 90 mol %, from 10 mol % to 85 mol %, or from 20 mol % to 80 mol % (or any fraction of these ranges) of the total lipid components.

The ratio of total lipid components to the payload (e.g., an encapsulated therapeutic agent such as a TREM (e.g., a TREM described herein) can be varied as desired. For example, the total lipid components to the payload (mass or weight) ratio can be from about 10:1 to about 30:1. In some embodiments, the total lipid components to the payload ratio (mass/mass ratio; w/w ratio) can be in the range of from about 1:1 to about 25:1, from about 10:1 to about 14:1, from about 3:1 to about 15:1, from about 4:1 to about 10:1, from about 5:1 to about 9:1, or about 6:1 to about 9:1. The amounts of total lipid components and the payload can be adjusted to provide a desired N/P ratio, for example, N/P ratio of 3, 4, 5, 6, 7, 8, 9, 10, 11, 12, 13, 14, 15, 16, 17, 18, 19, 20, 21, 22, 23, 24, 25, 26, 27, 28, 29, 30, or higher. Generally, the lipid-based carrier (or lipid nanoformulation's) overall lipid content can range from about 5 mg/ml to about 30 mg/mL. Nitrogen:phosphate ratios (N:P ratio) is evaluated at values between 0.1 and 100.

The efficiency of encapsulation of a payload such as a protein and/or nucleic acid, describes the amount of protein and/or nucleic acid that is encapsulated or otherwise associated with a lipid nanoformulation (e.g., liposome or LNP) after preparation, relative to the initial amount provided. The encapsulation efficiency is desirably high (e.g., at least 70%, 80%. 90%, 95%, close to 100%). The encapsulation efficiency may be measured, for example, by comparing the amount of protein or nucleic acid in a solution containing the liposome or LNP before and after breaking up the liposome or LNP with one or more organic solvents or detergents. An anion exchange resin may be used to measure the amount of free protein or nucleic acid (e.g., RNA) in a solution. Fluorescence may be used to measure the amount of free protein and/or nucleic acid (e.g., RNA) in a solution. For the lipid-based carrier (or lipid nanoformulation) described herein, the encapsulation efficiency of a protein and/or nucleic acid may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 70%. In some embodiments, the encapsulation efficiency may be at least 80%. In some embodiments, the encapsulation efficiency may be at least 90%. In some embodiments, the encapsulation efficiency may be at least 95%.

Delivery without a Carrier

A TREM, a TREM core fragment or a TREM fragment, a TREM composition or a pharmaceutical TREM composition described herein can be administered to a cell without a carrier, e.g., via naked delivery of the TREM, a TREM core fragment or a TREM fragment, a TREM composition or a pharmaceutical TREM composition.

In some embodiments, naked delivery as used herein refers to delivery without a carrier. In some embodiments, delivery without a carrier, e.g., naked delivery, comprises delivery with a moiety, e.g., a targeting peptide.

In some embodiments, a TREM, a TREM core fragment or a TREM fragment, or TREM composition, or pharmaceutical TREM composition described herein is delivered to a cell without a carrier, e.g., via naked delivery. In some embodiments, the delivery without a carrier, e.g., naked delivery, comprises delivery with a moiety, e.g., a targeting peptide.

### Use of TREMs

A TREM composition (e.g., a pharmaceutical TREM composition described herein) can modulate a function in a cell, tissue or subject. In embodiments, a TREM composition (e.g., a pharmaceutical TREM composition) described herein is contacted with a cell or tissue, or administered to a subject in need thereof, in an amount and for a time sufficient to modulate (increase or decrease) one or more of the following parameters: adaptor function (e.g., cognate or non-cognate adaptor function), e.g., the rate, efficiency, robustness, and/or specificity of initiation or elongation of a polypeptide chain; ribosome binding and/or occupancy; regulatory function (e.g., gene silencing or signaling); cell fate; mRNA stability; protein stability; protein transduction; protein compartmentalization. A parameter may be modulated, e.g., by at least 5% (e.g., at least 10%, 15%, 20%, 25%, 30%, 40%, 50%. 60%, 70%, 80%, 90%, 100%, 150%, 200% or more) compared to a reference tissue, cell or subject (e.g., a healthy, wild-type or control cell, tissue or subject).

All references and publications cited herein are hereby incorporated by reference.

The following examples are provided to further illustrate some embodiments of the present invention, but are not intended to limit the scope of the invention; it will be understood by their exemplary nature that other procedures, methodologies, or techniques known to those skilled in the art may alternatively be used.

### ENUMERATED EMBODIMENTS

1. A tRNA effector molecule (TREM) comprising a sequence of Formula (I):

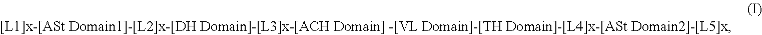

wherein:
independently, [L1] and [VL Domain], are optional;
   x is 0 or 1; and
   the TREM comprises a nucleotide substitution (e.g., a nucleotide mutation) in the TREM capable of modulating a functional parameter of the TREM.
2. The TREM of embodiment 1, wherein the functional parameter is activity of the TREM or stability of the TREM.
3. The TREM of any one of the preceding embodiments, wherein the functional parameter comprises premature termination codon (PTC) readthrough activity of the TREM (e.g., an increased level of PTC readthrough activity or decreased PTC readthrough activity), e.g., in a sample (e.g., a cell) or in a subject.
4. The TREM of any one of the preceding embodiments, wherein the functional parameter comprises stability of the TREM (e.g., increased stability of the TREM or decreased stability of the TREM), e.g., in a sample (e.g., a cell) or in a subject.
5. The TREM of any one of the preceding embodiments, wherein the functional parameter is selected from:
   (a) protein translation;
   (b) expression level (e.g., of polypeptide or protein, or mRNA);
   (c) post-translational modification of polypeptide or protein;
   (d) folding (e.g., of polypeptide or protein, or mRNA),
   (e) structure (e.g., of polypeptide or protein, or mRNA),
   (f) transduction (e.g., of polypeptide or protein),
   (g) compartmentalization (e.g., of polypeptide or protein, or mRNA),
   (h) incorporation (e.g., of polypeptide or protein, or mRNA) into a supermolecular structure, e.g., incorporation into a membrane, proteasome, or ribosome,
   (i) incorporation into a multimeric polypeptide, e.g., a homo or heterodimer, and/or
   (j) stability.
6. The TREM of embodiment 5, wherein the functional parameter further comprises:
   (1) modulation of a signaling pathway, e.g., a cellular signaling pathway which is downstream or upstream of the protein encoded by the endogenous ORF having a first sequence or PTC;
   (2) cell fate modulation;
   (3) ribosome occupancy modulation;
   (4) protein translation modulation;
   (5) mRNA stability modulation;
   (6) protein folding and structure modulation;
   (7) protein transduction or compartmentalization modulation; and/or
   (8) protein stability modulation.
7. The TREM of any one of the preceding embodiments, wherein the TREM comprises a TREM having at least 70%, 75%, 80%, 85%, 90%, 95%, or 99% sequence identity with a TREM provided in FIG. 3 or 23.
8. The TREM of any one of the preceding embodiments, wherein the TREM comprises a nucleotide sequence that differs by 2, 3, 4, 5, 6, 7, 8, 9, 10, 11, 12, 13, 14, 15, 16, 17, 18, 19, or nucleotides from the nucleotide sequence of a TREM provided in FIG. 3 or 23.
9. The TREM of any one of the preceding embodiments, wherein the TREM comprises a nucleotide sequence that differs by 10, 15, 20, 25, 30, 35 or 40 nucleotides from the nucleotide sequence of a TREM provided in FIG. 3 or 23.
10. The TREM of any one of the preceding embodiments, wherein the TREM comprises a nucleotide sequence that differs by more than 5 nucleotides from the nucleotide sequence of a TREM provided in FIG. 3 or 23.
11. The TREM of any one of the preceding embodiments, wherein the TREM comprises a nucleotide sequence that differs by more than 10 nucleotides from the nucleotide sequence of a TREM provided in FIG. 3 or 23.
12. The TREM of any one of the preceding embodiments, wherein the TREM comprises a sequence that differs by no more than 1, 2, 3, 4, 5, 6, 7, 8, 9, or 10 nucleotides from the nucleotide sequence of a TREM provided in FIG. 3 or 23.
13. The TREM of any one of the preceding embodiments, wherein the TREM comprises a nucleotide sequence that differs by no more than 10, 15, 20, 25, 30, 35 or 40 nucleotides from the nucleotide sequence of a TREM provided in FIG. 3 or 23.
14. The TREM of any one of the preceding embodiments, wherein the TREM comprises a nucleotide sequence that differs by no more than 1 nucleotide from the nucleotide sequence of a TREM provided in FIG. 3 or 23.
15. The TREM of any one of the preceding embodiments, wherein the TREM comprises a nucleotide sequence that differs by no more than 5 nucleotides from the nucleotide sequence of a TREM provided in FIG. 3 or 23.
16. The TREM of any one of the preceding embodiments, wherein the TREM comprises a nucleotide sequence that differs by no more than 10 nucleotides from the nucleotide sequence of a TREM provided in FIG. 3 or 23.
17. The TREM of any one of the preceding embodiments, wherein the TREM comprises a nucleotide substitution within the [ASt Domain1], e.g., the nucleotide substitution is at any of positions 1, 2, 3, 4, 5, 6, 7, 8, or 9 within the [ASt Domain1].
18. The TREM of any one of the preceding embodiments, wherein the TREM comprises a nucleotide substitution within the [DH Domain], e.g., the nucleotide substitution is at any of positions 1, 2, 3, 4, 5, 6, 7, 8, or 9 within the [DH Domain].
19. The TREM of any one of the preceding embodiments, wherein the TREM comprises a nucleotide substitution within the [ACH Domain], e.g., the nucleotide substitution is at any of positions 1, 2, 3, 4, 5, 6, 7, 8, or 9 within the [ACH Domain].
20. The TREM of any one of the preceding embodiments, wherein the TREM comprises a nucleotide substitution within the [VL Domain], e.g., the nucleotide substitution is at any of positions 1, 2, 3, 4, 5, 6, 7, 8, or 9 within the [VL Domain].
21. The TREM of any one of the preceding embodiments, wherein the TREM comprises a nucleotide substitution within the [TH Domain], e.g., the nucleotide substitution is at any of positions 1, 2, 3, 4, 5, 6, 7, 8, or 9 within the [TH Domain].
22. The TREM of any one of the preceding embodiments, wherein the TREM comprises a nucleotide substitution within the [ASt Domain2], e.g., the nucleotide substitution is at any of positions 1, 2, 3, 4, 5, 6, 7, 8, or 9 within the [ASt Domain2].
23. The TREM of any one of the preceding embodiments, wherein the TREM comprises a nucleotide substitution within the [DH Domain], e.g., the nucleotide substitution is at any of positions 10, 11, 12, 13, 14, 15, 16, 17, 18, 19, 20, 21, 22, 23, 24, 25, or 26 within the [DH Domain].
24. The TREM of any one of the preceding embodiments, wherein the TREM comprises a nucleotide substitution within the [ACH Domain], e.g., the nucleotide substitution is at any of positions 27, 28, 29, 30, 31, 32, 33, 34, 35, 36, 37, 38, 39, 40, 41, 42, or 43 within the [ACH Domain].
25. The TREM of any one of the preceding embodiments, wherein the TREM comprises a nucleotide substitution within the [VL Domain], e.g., the nucleotide substitution is at any of positions 44, 45, 46, 47, 48, or 49 within the [VL Domain].
26. The TREM of any one of the preceding embodiments, wherein the TREM comprises a nucleotide substitution within the [TH Domain], e.g., the nucleotide substitution is at any of positions 50, 51, 52, 53, 54, 55, 56, 57, 58, 59, 60, 61, 62, 63, or 64 within the [TH Domain].
27. The TREM of any one of the preceding embodiments, wherein the TREM comprises a nucleotide substitution within the [ASt Domain2], e.g., the nucleotide substitution is at any of positions 65, 66, 67, 68, 69, 70, 71, 72, 73, 74, 75, or 76 within the [ASt Domain2].
28. The TREM of any one of the preceding embodiments, wherein the TREM has a sequence selected from a sequence provided in FIG. 3 or 23.
29. The TREM of any one of the preceding embodiments, wherein the TREM comprises a plurality of nucleotide substitutions.
30. The TREM of any one of any one of the preceding embodiments, wherein the nucleotide substitution comprises substituting a first naturally occurring nucleotide with an adenosine, guanosine, cytosine, or uracil nucleotide.
31. The TREM of any one of the preceding embodiments, wherein the TREM is selected from SEQ ID NOs. 625-700, 701-800, 801-900, 901-1000, 1001-1100, 1101-1151, 1290-1300, 1301-1400, or 1401-1456 in FIG. 3.
32. The TREM of any one of the preceding embodiments, wherein the TREM is selected from SEQ ID NOs: 1457-1500, 1501-1600, 1601-1700, 1701-1800, 1801-1900, 1901-2000, or 2001-2032 in FIG. 23.
33. The TREM of any one of the preceding embodiments, wherein the TREM comprising a nucleotide substitution does not exhibit a difference in a functional parameter from a TREM in FIG. 3 or 23.
34. The TREM of any one of embodiments 1-26, wherein the TREM comprising a nucleotide substitution exhibits a difference in a functional parameter from a TREM in FIG. 3 or 23.
35. The TREM of any one of the preceding embodiments, wherein the TREM comprising a nucleotide substitution exhibits an improvement in a functional parameter from a TREM in FIG. 3 or 23.
36. The TREM of any one of the preceding embodiments, wherein the improvement comprises an improvement of about 5%, 10%, 15%, 20%, 25%, 30%, 35%, 40%, 45%, 50%,
55%, 60%, 65%, 70%, 75%, 80%, 85%, 90%, 95%, or greater, e.g., compared with the functional parameter of a TREM provided in FIG. 3 or 23.
37. The TREM of any one of the preceding embodiments, wherein the TREM comprising a nucleotide substitution exhibits a reduction in a functional parameter from a TREM in FIG. 3 or 23.
38. The TREM of any one of the preceding embodiments, wherein the reduction comprises a reduction of about 5%, 10%, 15%, 20%, 25%, 30%, 35%, 40%, 45%, 50%, 55%, 60%, 65%, 70%, 75%, 80%, 85%, 90%, 95%, or greater, e.g., compared with the functional parameter of a TREM provided in FIG. 3 or 23.
39. The TREM of any one of the preceding embodiments, wherein the TREM comprises a nucleotide substitution at any of positions 1, 2, 3, 4, 5, 6, or 7, e.g., within the [ASt Domain1].
40. The TREM of any one of the preceding embodiments, wherein the TREM comprises a nucleotide substitution at any of positions 16, 17, or 20a, e.g., within the [DH Domain].
41. The TREM of any one of the preceding embodiments, wherein the TREM comprises a nucleotide substitution at any of positions 27, 28, 29, 30, 31, 39, 40, 41, 42, or 43, e.g., within the [ACH Domain].
42. The TREM of any one of the preceding embodiments, wherein the TREM comprises a nucleotide substitution at any of positions 51, 59, 60, or 63, e.g., within the [TH Domain].
43. The TREM of any one of the preceding embodiments, wherein the TREM comprises a nucleotide substitution at any of positions 66, 67, 68, 69, 70, 71, 72, or 73, e.g., within the [ASt Domain2].
44. The TREM of any one of the preceding embodiments, wherein the TREM comprises a nucleotide substitution at any of positions 32, 33, 34, 35, 36, 37, or 38, e.g., within the [ACH Domain].
45. The TREM of any one of the preceding embodiments, wherein the TREM comprising a nucleotide substitution at any of positions 1, 2, 3, 4, 5, 6, or 7, e.g., within the [ASt Domain1], exhibits an improvement in a functional parameter of the TREM.
46. The TREM of any one of the preceding embodiments, wherein the TREM comprising a nucleotide substitution at any of positions 16, 17, or 20a, e.g., within the [DH Domain], exhibits an improvement in a functional parameter of the TREM.
47. The TREM of any one of the preceding embodiments, wherein the TREM comprising a nucleotide substitution at any of positions 27, 28, 29, 30, 31, 39, 40, 41, 42, or 43, e.g., within the [ACH Domain], exhibits an improvement in a functional parameter of the TREM.
48. The TREM of any one of the preceding embodiments, wherein the TREM comprising a nucleotide substitution at any of positions 51, 59, 60, or 63, e.g., within the [TH Domain], exhibits an improvement in a functional parameter of the TREM.
49. The TREM of any one of the preceding embodiments, wherein the TREM comprising a nucleotide substitution at any of positions 66, 67, 68, 69, 70, 71, 72, or 73, e.g., within the [ASt Domain2], exhibits an improvement in a functional parameter of the TREM.
50. The TREM of any one of the preceding embodiments, wherein the TREM comprising a nucleotide substitution at any of positions 32, 33, 34, 35, 36, 37, or 38, e.g., within the [ACH Domain], exhibits a reduction in a functional parameter of the TREM.
51. The TREM of any one of the preceding embodiments, wherein the TREM comprising a nucleotide substitution comprises a scaffold, e.g., an Arg-TGA, Gln-TAG, Glu-TAG, Leu-TAG, Tyr-TAG, Ser-TAG, or Lys-TAG scaffold, e.g., a TREM corresponding to a reference sequence of SEQ ID NO: 1638, 1835, 1660, 1654, 1867, 2000, 2001, 2016, 2017, 2022, 2023, 2020, 2021, 2018, or 2019.
52. The TREM of any one of the preceding embodiments, wherein the TREM comprising a nucleotide substitution corresponds to a scaffold, e.g., an Arg-TGA, Gln-TAG, Glu-TAG, Leu-TAG, Tyr-TAG, Ser-TAG, or Lys-TAG scaffold, e.g., a scaffold corresponding to a reference sequence of SEQ ID NO: 1638, 1835, 1660, 1654, 1867, 2000, 2001, 2016, 2017, 2022, 2023, 2020, 2021, 2018, or 2019.
53. The TREM of any one of the preceding embodiments, wherein the TREM comprising a nucleotide substitution corresponds to an Arg-TGA scaffold, e.g., corresponds to a reference sequence of SEQ ID NO: 1638.
54. The TREM of any one of the preceding embodiments, wherein the TREM comprising a nucleotide substitution corresponds to a Gln-TAG scaffold, e.g., corresponds to a reference sequence of SEQ ID NO: 1835, 1660, or 1654.
55. The TREM of any one of the preceding embodiments, wherein the TREM comprising a nucleotide substitution corresponds to a Glu-TAG scaffold, e.g., corresponds to a reference sequence of SEQ ID NO: 1867, 2000, or 2001.
56. The TREM of any one of the preceding embodiments, wherein the TREM comprising a nucleotide substitution corresponds to a Leu-TAG scaffold, e.g., corresponds to a reference sequence of SEQ ID NO: 2016 or 2017.
57. The TREM of any one of the preceding embodiments, wherein the TREM comprising a nucleotide substitution corresponds to a Tyr-TAG scaffold, e.g., corresponds to a reference sequence of SEQ ID NO: 2022 or 2023.
58. The TREM of any one of the preceding embodiments, wherein the TREM comprising a nucleotide substitution corresponds to a Ser-TAG scaffold, e.g., corresponds to a reference sequence of SEQ ID NO: 2020 or 2021.
59. The TREM of any one of the preceding embodiments, wherein the TREM comprising a nucleotide substitution corresponds to a Lys-TAG scaffold, e.g., corresponds to a reference sequence of SEQ ID NO: 2018 or 2019.
60. The TREM of any one of the preceding embodiments, wherein the TREM comprising a nucleotide substitution corresponding to a reference sequence of SEQ ID NO: 1638, 1835, 1660, 1654, 1867, 2000, 2001, 2016, 2017, 2022, 2023, 2020, 2021, 2018, or 2019, exhibits an improvement of a functional parameter of the TREM, e.g., relative to the functional parameter of the reference sequence.
61. The TREM of any one of the preceding embodiments, wherein the TREM comprising a nucleotide substitution corresponding to an Arg-TGA reference sequence, e.g., SEQ ID NO: 1638, exhibits an improvement of a functional parameter of the TREM, e.g., relative to the functional parameter of the reference sequence.
62. The TREM of any one of the preceding embodiments, wherein the TREM comprising a nucleotide substitution at any of nucleotide positions 1, 2, 3, 4, 5, 6, or 7, e.g., in the [ASt Domain1], corresponding to an Arg-TGA reference sequence, e.g., SEQ ID NO: 1638, exhibits an improvement of a functional parameter of the TREM, e.g., relative to the functional parameter of the reference sequence.
63. The TREM of any one of the preceding embodiments, wherein the TREM comprising a nucleotide substitution at any of nucleotide positions 32, 33, 34, 35, 36, 37, or 38, e.g., in the [ACH Domain], corresponding to an Arg-TGA reference sequence, e.g., SEQ ID NO: 1638, exhibits a reduction of a functional parameter of the TREM, e.g., relative to the functional parameter of the reference sequence.
64. The TREM of any one of the preceding embodiments, wherein the TREM comprising a nucleotide substitution at any of nucleotide positions 65, 66, 67, 68, 69, 70, 71, 72, or 73, e.g., in the [ASt Domain2], corresponding to an Arg-TGA reference sequence, e.g., SEQ ID NO: 1638, exhibits an improvement of a functional parameter of the TREM, e.g., relative to the functional parameter of the reference sequence.
65. The TREM of any one of the preceding embodiments, wherein the TREM comprising a nucleotide substitution corresponding to a Gln-TAG reference sequence, e.g., SEQ ID NO: 1835, 1660, or 1654, exhibits an improvement of a functional parameter of the TREM, e.g., relative to the functional parameter of the reference sequence.
66. The TREM of any one of the preceding embodiments, wherein the TREM comprising a nucleotide substitution at any of nucleotide positions 26, 27, 28, 29, 30, or 31, e.g., in the [ACH Domain], corresponding to a Gln-TAG reference sequence, e.g., SEQ ID NO: 1835, 1660, or 1654, exhibits an improvement of a functional parameter of the TREM, e.g., relative to the functional parameter of the reference sequence.
67. The TREM of any one of the preceding embodiments, wherein the TREM comprising a nucleotide substitution at any of nucleotide positions 32, 33, 34, 35, 36, 37, or 38, e.g., in the [ACH Domain], corresponding to a Gln-TAG reference sequence, e.g., SEQ ID NO: 1835, 1660, or 1654, exhibits a reduction of a functional parameter of the TREM, e.g., relative to the functional parameter of the reference sequence.
68. The TREM of any one of the preceding embodiments, wherein the TREM comprising a nucleotide substitution at any of nucleotide positions 39, 40, 41, 42, 43, or 44, e.g., in the [ACH Domain], corresponding to a Gln-TAG reference sequence, e.g., SEQ ID NO: 1835, 1660, or 1654, exhibits an improvement of a functional parameter of the TREM, e.g., relative to the functional parameter of the reference sequence.
69. The TREM of any one of the preceding embodiments, wherein the TREM comprising a nucleotide substitution corresponding to a Glu-TAG reference sequence, e.g., SEQ ID NO: 1867, 2000, or 2001, exhibits an improvement of a functional parameter of the TREM, e.g., relative to the functional parameter of the reference sequence.
70. The TREM of any one of the preceding embodiments, wherein the TREM comprising a nucleotide substitution at any of nucleotide positions 1, 2, 3, 4, 5, 6, 7, 8, 9, 10, 11, 12, 13, 14, or 15, e.g., in the [ASt Domain1] or the [DH Domain], corresponding to a Glu-TAG reference sequence, e.g., SEQ ID NO: 1867, 2000, or 2001, exhibits an improvement of a functional parameter of the TREM, e.g., relative to the functional parameter of the reference sequence.
71. The TREM of any one of the preceding embodiments, wherein the TREM comprising a nucleotide substitution at any of nucleotide positions 20a, 21, 22, 23, 24, 25, 26, 27, 28, 29, 30, or 31, e.g., in the [DH Domain] or the [ACH Domain], corresponding to a Glu-TAG reference sequence, e.g., SEQ ID NO: 1867, 2000, or 2001, exhibits an improvement of a functional parameter of the TREM, e.g., relative to the functional parameter of the reference sequence.
72. The TREM of any one of the preceding embodiments, wherein the TREM comprising a nucleotide substitution at any of nucleotide positions 32, 33, 34, 35, 36, 37, or 38, e.g., in the [ACH Domain], corresponding to a Glu-TAG reference sequence, e.g., SEQ ID NO: 1867, 2000, or 2001, exhibits a reduction of a functional parameter of the TREM, e.g., relative to the functional parameter of the reference sequence.
73. The TREM of any one of the preceding embodiments, wherein the TREM comprising a nucleotide substitution at any of nucleotide positions 40, 41, 42, 43, 44, or 45, e.g., in the [ACH Domain], corresponding to a Glu-TAG reference sequence, e.g., SEQ ID NO: 1867, 2000, or 2001, exhibits an improvement of a functional parameter of the TREM, e.g., relative to the functional parameter of the reference sequence.
74. The TREM of any one of the preceding embodiments, wherein the TREM comprising a nucleotide substitution at any of nucleotide positions 46 or 48, e.g., in the [VL Domain], corresponding to a Glu-TAG reference sequence, e.g., SEQ ID NO: 1867, 2000, or 2001, exhibits an improvement of a functional parameter of the TREM, e.g., relative to the functional parameter of the reference sequence.
75. The TREM of any one of the preceding embodiments, wherein the TREM comprising a nucleotide substitution at any of nucleotide positions 59 or 60, e.g., in the [TH Domain], corresponding to a Glu-TAG reference sequence, e.g., SEQ ID NO: 1867, 2000, or 2001, exhibits an improvement of a functional parameter of the TREM, e.g., relative to the functional parameter of the reference sequence.
76. The TREM of any one of the preceding embodiments, wherein the TREM comprising a nucleotide substitution at any of nucleotide positions 66, 67, 68, 69, 70, 71, 72, or 73, e.g., in the [ASt Domain2], corresponding to a Glu-TAG reference sequence, e.g., SEQ ID NO: 1867, 2000, or 2001, exhibits an improvement of a functional parameter of the TREM, e.g., relative to the functional parameter of the reference sequence.
77. The TREM of any one of the preceding embodiments, wherein the TREM comprising a nucleotide substitution corresponding to a Leu-TAG reference sequence, e.g., SEQ ID NO: 2016 or 2017, exhibits an improvement of a functional parameter of the TREM, e.g., relative to the functional parameter of the reference sequence.
78. The TREM of any one of the preceding embodiments, wherein the TREM comprising a nucleotide substitution at any of nucleotide positions 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30, e.g., in the [ASt Domain1], the [DH Domain], or the [ACH Domain], corresponding to a Leu-TAG reference sequence, e.g., SEQ ID NO: 2016 or 2017, exhibits an improvement of a functional parameter of the TREM, e.g., relative to the functional parameter of the reference sequence.
79. The TREM of any one of the preceding embodiments, wherein the TREM comprising a nucleotide substitution at any of nucleotide positions 32, 33, 34, 35, 36, 37, or 38, e.g., in the [ACH Domain], corresponding to a Leu-TAG reference sequence, e.g., SEQ ID NO: 2016 or 2017, exhibits a reduction of a functional parameter of the TREM, e.g., relative to the functional parameter of the reference sequence.
80. The TREM of any one of the preceding embodiments, wherein the TREM comprising a nucleotide substitution at any of nucleotide positions 39, 40, 41, 42, 43, 44, 45, V11, V12, V13, V14, V15, V16, V17, V1, V2, V3, V4, V5, V27, V26, V25, V24, V23, V22, V21, 46, 47, 48, 49, or 50, e.g., in the [ACH Domain], the [VL Domain], or the [TH Domain], corresponding to a Leu-TAG reference sequence, e.g., SEQ ID NO: 2016 or 2017, exhibits an improvement of a functional parameter of the TREM, e.g., relative to the functional parameter of the reference sequence.
81. The TREM of any one of the preceding embodiments, wherein the TREM comprising a nucleotide substitution at any of nucleotide positions 64, 65, 66, 67, 68, 69, 70, 71, 72, or 73, e.g., in the [TH Domain] or the [ASt Domain2], corresponding to a Leu-TAG reference sequence, e.g., SEQ ID NO: 2016 or 2017, exhibits an improvement of a functional parameter of the TREM, e.g., relative to the functional parameter of the reference sequence.
82. The TREM of any one of the preceding embodiments, wherein the TREM comprising a nucleotide substitution corresponding to a Tyr-TAG reference sequence, e.g., SEQ ID NO: 2022 or 2023, exhibits an improvement of a functional parameter of the TREM, e.g., relative to the functional parameter of the reference sequence.
83. The TREM of any one of the preceding embodiments, wherein the TREM comprising a nucleotide substitution at any of nucleotide positions 4, 5, 6, or 7, e.g., in the [ASt Domain1], corresponding to a Tyr-TAG reference sequence, e.g., SEQ ID NO: 2022 or 2023, exhibits an improvement of a functional parameter of the TREM, e.g., relative to the functional parameter of the reference sequence.
84. The TREM of any one of the preceding embodiments, wherein the TREM comprising a nucleotide substitution at any of nucleotide positions 16, 17, or 20, e.g., in the [DH Domain], corresponding to a Tyr-TAG reference sequence, e.g., SEQ ID NO: 2022 or 2023, exhibits an improvement of a functional parameter of the TREM, e.g., relative to the functional parameter of the reference sequence.
85. The TREM of any one of the preceding embodiments, wherein the TREM comprising a nucleotide substitution at any of nucleotide positions 28, 29, or 30, e.g., in the [ACH Domain], corresponding to a Tyr-TAG reference sequence, e.g., SEQ ID NO: 2022 or 2023, exhibits an improvement of a functional parameter of the TREM, e.g., relative to the functional parameter of the reference sequence.
86. The TREM of any one of the preceding embodiments, wherein the TREM comprising a nucleotide substitution at any of nucleotide positions 32, 33, 34, 35, 36, 37, or 38, e.g., in the [ACH Domain], corresponding to a Tyr-TAG reference sequence, e.g., SEQ ID NO: 2022 or 2023, exhibits a reduction of a functional parameter of the TREM, e.g., relative to the functional parameter of the reference sequence.
87. The TREM of any one of the preceding embodiments, wherein the TREM comprising a nucleotide substitution at any of nucleotide positions 40, 41, 42, 43, or 44, e.g., in the [ACH Domain], corresponding to a Tyr-TAG reference sequence, e.g., SEQ ID NO: 2022 or 2023, exhibits an improvement of a functional parameter of the TREM, e.g., relative to the functional parameter of the reference sequence.
88. The TREM of any one of the preceding embodiments, wherein the TREM comprising a nucleotide substitution at any of nucleotide positions 47, 48, 49, 50, or 51, e.g., in the [VL Domain] or the [TH Domain], corresponding to a Tyr-TAG reference sequence, e.g., SEQ ID NO: 2022 or 2023, exhibits an improvement of a functional parameter of the TREM, e.g., relative to the functional parameter of the reference sequence.
89. The TREM of any one of the preceding embodiments, wherein the TREM comprising a nucleotide substitution at any of nucleotide positions 59, 60, 61, 62, 63, 64, 65, 66, 67, 68, 69, or 70, e.g., in the [TH Domain] or the [ASt Domain2], corresponding to a Tyr-TAG reference sequence, e.g., SEQ ID NO: 2022 or 2023, exhibits an improvement of a functional parameter of the TREM, e.g., relative to the functional parameter of the reference sequence.
90. The TREM of any one of the preceding embodiments, wherein the TREM comprising a nucleotide substitution corresponding to a Ser-TAG reference sequence, e.g., SEQ ID NO: 2020 or 2021, exhibits an improvement of a functional parameter of the TREM, e.g., relative to the functional parameter of the reference sequence.
91. The TREM of any one of the preceding embodiments, wherein the TREM comprising a nucleotide substitution at any of nucleotide positions 3, 4, 5, or 6, e.g., in the [ASt Domain1], corresponding to a Ser-TAG reference sequence, e.g., SEQ ID NO: 2020 or 2021, exhibits an improvement of a functional parameter of the TREM, e.g., relative to the functional parameter of the reference sequence.
92. The TREM of any one of the preceding embodiments, wherein the TREM comprising a nucleotide substitution at any of nucleotide positions 29, 30 31, 32, or 33, e.g., in the [ACH Domain], corresponding to a Ser-TAG reference sequence, e.g., SEQ ID NO: 2020 or 2021, exhibits an improvement of a functional parameter of the TREM, e.g., relative to the functional parameter of the reference sequence.
93. The TREM of any one of the preceding embodiments, wherein the TREM comprising a nucleotide substitution at any of nucleotide positions 34, 35, or 36, e.g., in the [ACH Domain], corresponding to a Ser-TAG reference sequence, e.g., SEQ ID NO: 2020 or 2021, exhibits an improvement of a functional parameter of the TREM, e.g., relative to the functional parameter of the reference sequence.
94. The TREM of any one of the preceding embodiments, wherein the TREM comprising a nucleotide substitution at any of nucleotide positions 37, 38, 39, 40, or 41, e.g., in the [ACH Domain], corresponding to a Ser-TAG reference sequence, e.g., SEQ ID NO: 2020 or 2021, exhibits an improvement of a functional parameter of the TREM, e.g., relative to the functional parameter of the reference sequence.
95. The TREM of any one of the preceding embodiments, wherein the TREM comprising a nucleotide substitution at any of nucleotide positions 51 or 63, e.g., in the [ACH Domain], corresponding to a Ser-TAG reference sequence, e.g., SEQ ID NO: 2020 or 2021, exhibits an improvement of a functional parameter of the TREM, e.g., relative to the functional parameter of the reference sequence.
96. The TREM of any one of the preceding embodiments, wherein the TREM comprising a nucleotide substitution at position 67, e.g., in the [ASt Domain2], corresponding to a Ser-TAG reference sequence, e.g., SEQ ID NO: 2020 or 2021, exhibits an improvement of a functional parameter of the TREM, e.g., relative to the functional parameter of the reference sequence.
97. The TREM of any one of the preceding embodiments, wherein the TREM comprising a nucleotide substitution corresponding to a Lys-TAG reference sequence, e.g., SEQ ID NO: 2018 or 2019, exhibits an improvement of a functional parameter of the TREM, e.g., relative to the functional parameter of the reference sequence.
98. The TREM of any one of the preceding embodiments, wherein the TREM comprising a nucleotide substitution at any of nucleotide positions 1, 2, 3, 4, 5, 6, or 7, e.g., in the [ASt Domain1], corresponding to a Lys-TAG reference sequence, e.g., SEQ ID NO: 2018 or 2019, exhibits an improvement of a functional parameter of the TREM, e.g., relative to the functional parameter of the reference sequence.
99. The TREM of any one of the preceding embodiments, wherein the TREM comprising a nucleotide substitution at any of nucleotide positions 17 or 18, e.g., in the [DH Domain], corresponding to a Lys-TAG reference sequence, e.g., SEQ ID NO: 2018 or 2019, exhibits an improvement of a functional parameter of the TREM, e.g., relative to the functional parameter of the reference sequence.
100. The TREM of any one of the preceding embodiments, wherein the TREM comprising a nucleotide substitution at any of nucleotide positions 32, 33, 34, 35, 36, 37, or 38, e.g., in the [ACH Domain], corresponding to a Lys-TAG reference sequence, e.g., SEQ ID NO: 2018 or 2019, exhibits a reduction of a functional parameter of the TREM, e.g., relative to the functional parameter of the reference sequence.
101. The TREM of any one of the preceding embodiments, wherein the TREM comprising a nucleotide substitution at position 37, e.g., in the [ACH Domain], corresponding to a Lys-TAG reference sequence, e.g., SEQ ID NO: 2018 or 2019, exhibits an improvement of a functional parameter of the TREM, e.g., relative to the functional parameter of the reference sequence.
102. The TREM of any one of the preceding embodiments, wherein the TREM comprising a nucleotide substitution at position 50, e.g., in the [TH Domain], corresponding to a Lys-TAG reference sequence, e.g., SEQ ID NO: 2018 or 2019, exhibits an improvement of a functional parameter of the TREM, e.g., relative to the functional parameter of the reference sequence.
103. The TREM of any one of the preceding embodiments, wherein the TREM comprising a nucleotide substitution at any of nucleotide positions 64, 65, 66, 67, 68, 69, 70, 71, 72, or 73, e.g., in the [DH Domain] or the [ASt Domain2], corresponding to a Lys-TAG reference sequence, e.g., SEQ ID NO: 2018 or 2019, exhibits an improvement of a functional parameter of the TREM, e.g., relative to the functional parameter of the reference sequence.
104. The TREM of any one of the preceding embodiments, wherein the TREM comprising a nucleotide substitution corresponds to a reference sequence of SEQ ID NO: 1638, 1835, 1660, 1654, 1867, 2000, 2001, 2016, 2017, 2022, 2023, 2020, 2021, 2018, or2019.
105. The TREM of any one of the preceding embodiments, wherein the TREM comprises a non-naturally occurring modification.
106. The TREM of embodiment 105, wherein the non-naturally occurring modification is present on the 2′-position of a nucleotide sugar or within the internucleotide region (e.g., a backbone modification).
107. The TREM of any one of embodiments 105-106, wherein the non-naturally occurring modification is selected from a 2′-O-methyl (2-OMe), 2′-halo (e.g., 2′F or 2′C1), 2′-O-methoxyethyl (2′MOE), or 2′deoxy modification.
108. The TREM of any one of embodiments 105-107, wherein the non-naturally occurring modification is a phosphorothioate modification.
109. The TREM of any one of embodiments 105-108, wherein the TREM comprises 1, 2, 3, 4, 5, 6, 7, 8, 9, or 10 additional non-naturally occurring modifications compared with a TREM provided in FIG. 3 (e.g., 2′-ribose modifications or an internucleotide modification, e.g., 2′OMe, 2′-halo, 2′-MOE, 2′-deoxy, or phosphorothiorate modifications).
110. The TREM of any one of the preceding embodiments, wherein the TREM comprises a pattern of non-naturally occurring modifications selected from the patterns listed in Table 6.
111. The TREM of any one of the preceding embodiments, wherein:
(i) the non-naturally occurring modification is present at a nucleotide position which corresponds to one or more of nucleotides 1-9 of SEQ ID NO: 734; and/or
   (ii) the TREM differs from the nucleotide sequence of SEQ ID NO: 734 by about 1, 2, 3, 4, 5, 6, 7, 8, 9, or 10 nucleotides.
112. The TREM of any one of the preceding embodiments, wherein:
(i) the non-naturally occurring modification is present at a nucleotide position which corresponds to one or more of nucleotides 10-19 of SEQ ID NO: 734; and/or
   (ii) the TREM differs from the nucleotide sequence of SEQ ID NO: 734 by about 1, 2, 3, 4, 5, 6, 7, 8, 9, or 10 nucleotides.
113. The TREM of any one of the preceding embodiments, wherein:
(i) the non-naturally occurring modification is present at a nucleotide position which corresponds to one or more of nucleotides 20-29 of SEQ ID NO: 734; and/or
   (ii) the TREM differs from the nucleotide sequence of SEQ ID NO: 734 by about 1, 2, 3, 4, 5, 6, 7, 8, 9, or 10 nucleotides.
114. The TREM of any one of the preceding embodiments, wherein:
(i) the non-naturally occurring modification is present at a nucleotide position which corresponds to one or more of nucleotides 30-39 of SEQ ID NO: 734; and/or
   (ii) the TREM differs from the nucleotide sequence of SEQ ID NO: 734 by about 1, 2, 3, 4, 5, 6, 7, 8, 9, or 10 nucleotides.
115. The TREM of any one of the preceding embodiments, wherein:
(i) the non-naturally occurring modification is present at a nucleotide position which corresponds to one or more of nucleotides 40-49 of SEQ ID NO: 734; and/or
   (ii) the TREM differs from the nucleotide sequence of SEQ ID NO: 734 by about 1, 2, 3, 4, 5, 6, 7, 8, 9, or 10 nucleotides.
116. The TREM of any one of the preceding embodiments, wherein:
(i) the non-naturally occurring modification is present at a nucleotide position which corresponds to one or more of nucleotides 50-59 of SEQ ID NO: 734; and/or
   (ii) the TREM differs from the nucleotide sequence of SEQ ID NO: 734 by about 1, 2, 3, 4, 5, 6, 7, 8, 9, or 10 nucleotides.
117. The TREM of any one of the preceding embodiments, wherein:
(i) the non-naturally occurring modification is present at a nucleotide position which corresponds to one or more of nucleotides 60-69 of SEQ ID NO: 734; and/or
   (ii) the TREM differs from the nucleotide sequence of SEQ ID NO: 734 by about 1, 2, 3, 4, 5, 6, 7, 8, 9, or 10 nucleotides.
118. The TREM of any one of the preceding embodiments, wherein:
(i) the non-naturally occurring modification is present at a nucleotide position which corresponds to one or more of nucleotides 70-76 of SEQ ID NO: 734; and/or
   (ii) the TREM differs from the nucleotide sequence of SEQ ID NO: 734 by about 1, 2, 3, 4, 5, 6, 7, 8, 9, or 10 nucleotides.
119. The TREM of any one of the preceding embodiments, wherein the TREM comprises the nucleotide sequence of SEQ ID NO: 743.
120. The TREM of any one of the preceding embodiments, wherein the TREM comprises the nucleotide sequence of SEQ ID NO: 652.
121. The TREM of any one of the preceding embodiments, wherein the TREM comprises the nucleotide sequence of SEQ ID NO: 702.
122. The TREM of any one of the preceding embodiments, wherein the TREM comprises the nucleotide sequence of SEQ ID NO: 627.
123. The TREM of any one of the preceding embodiments, wherein the TREM comprises the nucleotide sequence of SEQ ID NO: 1150.
124. The TREM of any one of the preceding embodiments, wherein the TREM comprises the nucleotide sequence of SEQ ID NO: 1151.
125. The TREM of any one of the preceding embodiments, wherein the TREM comprises the nucleotide sequence of SEQ ID NO: 794.
126. The TREM of any one of the preceding embodiments, wherein the TREM comprises the nucleotide sequence of SEQ ID NO: 641.
127. The TREM of any one of the preceding embodiments, wherein the TREM comprises the nucleotide sequence of SEQ ID NO: 710.
128. The TREM of any one of the preceding embodiments, wherein the non-naturally occurring modification is present at a nucleotide position which corresponds to one or more of nucleotides 1-76, according to the CtNS.
129. A TREM (e.g., a TREM provided in Table 7) that has a pattern of non-naturally occurring modifications according to a pattern in Table 6 (e.g., a pattern selected from Pattern Nos. 1-32).
130. A TREM (e.g., a TREM provided in Table 7) that has the pattern of non-naturally occurring modifications according to Pattern No. 1 in Table 6.
131. A TREM (e.g., a TREM provided in Table 7) that has the pattern of non-naturally occurring modifications according to Pattern No. 2 in Table 6.
132. A TREM (e.g., a TREM provided in Table 7) that has the pattern of non-naturally occurring modifications according to Pattern No. 3 in Table 6.
133. A TREM (e.g., a TREM provided in Table 7) that has the pattern of non-naturally occurring modifications according to Pattern No. 4 in Table 6.
134. A TREM (e.g., a TREM provided in Table 7) that has the pattern of non-naturally occurring modifications according to Pattern No. 5 in Table 6.
135. A TREM (e.g., a TREM provided in Table 7) that has the pattern of non-naturally occurring modifications according to Pattern No. 6 in Table 6.
136. A TREM (e.g., a TREM provided in Table 7) that has the pattern of non-naturally occurring modifications according to Pattern No. 7 in Table 6.
137. A TREM (e.g., a TREM provided in Table 7) that has the pattern of non-naturally occurring modifications according to Pattern No. 8 in Table 6.
138. A TREM (e.g., a TREM provided in Table 7) that has the pattern of non-naturally occurring modifications according to Pattern No. 9 in Table 6.
139. A TREM (e.g., a TREM provided in Table 7) that has the pattern of non-naturally occurring modifications according to Pattern No. 10 in Table 6.
140. A TREM (e.g., a TREM provided in Table 7) that has the pattern of non-naturally occurring modifications according to Pattern No. 11 in Table 6.
141. A TREM (e.g., a TREM provided in Table 7) that has the pattern of non-naturally occurring modifications according to Pattern No. 12 in Table 6.
142. A TREM (e.g., a TREM provided in Table 7) that has the pattern of non-naturally occurring modifications according to Pattern No. 13 in Table 6.
143. A TREM (e.g., a TREM provided in Table 7) that has the pattern of non-naturally occurring modifications according to Pattern No. 14 in Table 6.
144. A TREM (e.g., a TREM provided in Table 7) that has the pattern of non-naturally occurring modifications according to Pattern No. 15 in Table 6.
145. A TREM (e.g., a TREM provided in Table 7) that has the pattern of non-naturally occurring modifications according to Pattern No. 16 in Table 6.
146. A TREM (e.g., a TREM provided in Table 7) that has the pattern of non-naturally occurring modifications according to Pattern No. 17 in Table 6.
147. A TREM (e.g., a TREM provided in Table 7) that has the pattern of non-naturally occurring modifications according to Pattern No. 18 in Table 6.
148. A TREM (e.g., a TREM provided in Table 7) that has the pattern of non-naturally occurring modifications according to Pattern No. 19 in Table 6.
149. A TREM (e.g., a TREM provided in Table 7) that has the pattern of non-naturally occurring modifications according to Pattern No. 20 in Table 6.
150. A TREM (e.g., a TREM provided in Table 7) that has the pattern of non-naturally occurring modifications according to Pattern No. 21 in Table 6.
151. A TREM (e.g., a TREM provided in Table 7) that has the pattern of non-naturally occurring modifications according to Pattern No. 22 in Table 6.
152. A TREM (e.g., a TREM provided in Table 7) that has the pattern of non-naturally occurring modifications according to Pattern No. 23 in Table 6.
153. A TREM (e.g., a TREM provided in Table 7) that has the pattern of non-naturally occurring modifications according to Pattern No. 24 in Table 6.
154. A TREM (e.g., a TREM provided in Table 7) that has the pattern of non-naturally occurring modifications according to Pattern No. 25 in Table 6.
155. A TREM (e.g., a TREM provided in Table 7) that has the pattern of non-naturally occurring modifications according to Pattern No. 26 in Table 6.
156. A TREM (e.g., a TREM provided in Table 7) that has the pattern of non-naturally occurring modifications according to Pattern No. 27 in Table 6.
157. A TREM (e.g., a TREM provided in Table 7) that has the pattern of non-naturally occurring modifications according to Pattern No. 28 in Table 6.
158. A TREM (e.g., a TREM provided in Table 7) that has the pattern of non-naturally occurring modifications according to Pattern No. 29 in Table 6.
159. A TREM (e.g., a TREM provided in Table 7) that has the pattern of non-naturally occurring modifications according to Pattern No. 30 in Table 6.
160. A TREM (e.g., a TREM provided in Table 7) that has the pattern of non-naturally occurring modifications according to Pattern No. 31 in Table 6.
161. A TREM (e.g., a TREM provided in Table 7) that has the pattern of non-naturally occurring modifications according to Pattern No. 32 in Table 6.
162. A pharmaceutical composition comprising a TREM of any one of the preceding embodiments.
163. The pharmaceutical composition of embodiment 162, further comprising a pharmaceutically acceptable component, e.g., an excipient.
164. A lipid nanoparticle formulation comprising a TREM of any one of embodiments 1-163.
165. A lipid nanoparticle formulation comprising a pharmaceutical composition of embodiment 164.
166. A method of treating a subject having a disease or disorder associated with a PTC comprising administering to the subject a TREM, TREM core fragment, or TREM fragment described herein (e.g., a TREM of any one of the preceding claims), thereby treating the subject having the disease or disorder.
167. The method of embodiment 166, wherein the disease or disorder associated with a PTC comprises Hemophilia B, Fabry disease, Usher syndrome, or CLN2 disease.
168. A method of modulating a functional parameter of a tRNA effector molecule (TREM), wherein the TREM comprises a sequence of Formula (I):

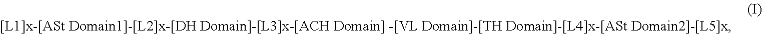

wherein:
independently, [L1] and [VL Domain], are optional;
x is 0 or 1; and
the TREM comprises a nucleotide substitution (e.g., a nucleotide mutation) in the TREM capable of modulating a functional parameter of the TREM;
thereby modulating the functional parameter of the TREM.

### EXAMPLES

Table of Contents for Examples

| | |
|---|---|
| Example 1 | Synthesis of modified TREMs |
| Example 2 | HPLC and MS analysis of modified TREMs |
| Example 3 | Analysis of modified TREMs via anion-exchange HPLC |
| Example 4 | Analysis of TREMs via PAGE Purification and Analysis |
| Example 5 | Deprotection of synthesized TREM |
| Example 6 | Characterization of chemically modified TREMs for |
| | readthrough of a premature termination codon (PTC) in a |
| | reporter protein |
| Example 7 | Characterization of chemically modified TREMs for |
| | readthrough of a premature termination codon (PTC) in a |
| | disease reporter cell line |
| Example 8 | Pooled screen for increased premature termination codon |
| | (PTC) readthrough activity of exemplary TREMs having |
| | nucleotide substitutions. |

### Example 1: Synthesis of Modified TREMS

Generally, TREM molecules (e.g., modified TREMs) may be chemically synthesized and purified by HPLC according to standard solid phase synthesis methods using phosphoramidite chemistry. (see, e.g., Scaringe S. et al. (2004) Curr Protoc Nucleic Acid Chem, 2.10.1-2.10.16; Usman N. et al. (1987) J. Am. Chem. Soc, 109, 7845-7854). Individually modified TREM molecules containing one or more 2′-methoxy (2′OMe), 2′fluoro (2′F), 2′-methoxyethyl (2′-MOE), or phosphorothioate (PS) modifications were prepared using TREM-Arg-TGA sequences as a framework according to phosphoramidite technology on solid phase used in oligonucleotide synthesis. For clarity, the arginine non-cognate TREM molecule named TREM-Arg-TGA contains the sequence of ARG-UCU-TREM body but with the anticodon sequence corresponding to UCA instead of UCU (i.e., SEQ ID NO: 622).

To make the 2′OMe modified TREMs, the following 2′-O-methyl phosphoramidites were used: (5′-O-dimethoxytrityl-N6-(benzoyl)-2′-O-methyl-adenosine-3′-O-(2-cyanoethyl-N,N-diisopropy-lamino) phosphoramidite, 5′-O-dimethoxy-trityl-N4-(acetyl)-2′-O-methyl-cytidine-3′-O-(2-cyanoethyl-N,N-diisopropyl-amino) phosphoramidite, (5′-O-dimethoxytrityl-N2-(isobutyryl)-2′-O-methyl-guanosine-3′-O-(2-cyano-ethyl-N,N-diisopropylamino)-phosphoramidite, and 5′-O-dimethoxy-trityl-2′-O-methyluridine-3′-O-(2-cyanoethyl-N,N-diisopropylamino)phosphoramidite. To make the 2′-deoxy and 2′-F modified TREMs, analogous 2′-deoxy and 2′-fluoro-phosphoramidites with the same protecting groups as the 2′-O-methyl RNA amidites were used. To make the 2′-MOE modified TREMs, the following 2′-MOE-phosphoramidites were used: 5′-O-(4,4′-Dimethoxytrityl)-2′-O-methoxyethyl-N6-benzoyl-adenosine-3′-O-[(2-cyanoethyl)-(N,N-diisopropyl)]-phosphoramidite, 5′-O-(4,4′-Dimethoxytrityl)-2′-O-methoxyethyl-5-methyl-N4-benzoyl- cytidine-3′-O-[(2-cyanoethyl)-(N,N-diisopropyl)]-phosphoramidite, 5′-O-(4,4′-Dimethoxytrityl)-2′-O-methoxyethyl-N2-isobutyryl-guanosine-3′-O-[(2-cyanoethyl)-(N,N-diisopropyl)]-phosphoramidite, 5′-O-(4,4′-Dimethoxytrityl)-2′-O-methoxyethyl-5-methyl-uridine-3′-O-[(2-cyanoethyl)-(N,N-diisopropyl)]-phosphoramidite.

During the oligonucleotide synthesis via this phosphoramidites approach, the phosphorothioate was introduced by oxidizing the phosphite triester using a sulfur transfer reagent, such as tetraethylthiuram disulfide (TETD), bis(O,O-diisopropoxy phosphinothioyl) disulfide (Stec's reagent), 3H-1,2-benzodithiol-3-one-1,1,-dioxide (Beaucage reagent), phenylacetyl disulfide (PADS), 3-ethoxy-1,2,4-dithiazoline-5-one (EDITH), 1,2-dithiazole-5-thione (xanthane hydride or ADTT), 3-((dimethylamino-methylidene)amino)-3H-1,2,4-dithiazole-3-thione (DDTT), dimethylthiuram disulfide (DTD), 3-phenyl-1,2,4-dithiazoline-5-one (PolyOrg Sulfa or POS).

FIG. 3 describes a series of singly and multiply modified TREMs synthesized according to this procedure. The sequences of each of these TREMs are provided in the table, wherein r: ribonucleotide; m: 2′-OMe; *: PS linkage; f: 2′-fluoro; moe: 2′-moe; d: deoxyribonucleotide; 5MeC: 5-methylcytosine. Thus, for example, mA represents 2′-O-methyl adenosine, moe5MeC represents 2′-MOE nucleotide with 5-methylcytosine nucleobase, and dA represents an adenosine deoxyribonucleotide.

### Example 2: HPLC and Ms Analysis of Modified Trems

Chemically modified TREM molecules may be analyzed by HPLC, for example, to evaluate the purity and homogeneity of the compositions. A Waters Aquity UPLC system using a Waters BEH C18 column (2.1 mm×50 mm×1.7 μm) may be used for this analysis. Samples may be prepared by dissolving 0.5 nmol of the TREM in 75 μL of water and injecting 2 L of the solution. The buffers used may be 50 mM dimethylhexylammonium acetate with 10% CH₃CN (acetonitrile) as buffer A and 50 mM dimethylhexylammonium acetate with 75% CH₃CN as buffer B (gradient 25-75% buffer B over 5 mins), with a flow rate of 0.5 mL/min at 60° C. ESI-LCMS data for the chemically modified TREMs may be acquired on a Thermo Ultimate 3000-LTQ-XL mass spectrometer.

FIG. 3 describes a series of singly and multiply modified TREMs synthesized according to the protocol outlined in Example 1. The calculated and detected molecular weights for each sequence were determined as outlined herein.

### Example 3: Analysis of Modified TREMs Via Anion-Exchange HPLC

This example describes the quality control of a synthesized TREM via anion-exchange HPLC. Using the Dionex DNA-Pac-PA-100 column, a gradient is employed using HPLC buffer A and HPLC buffer B. 0.5 ODUs of a sample that has been dissolved in H2O or Tris buffer, pH 7.5 is injected onto the gradient. The gradient employed is based on oligonucleotide length and can be applied according to Table 13. The parameters provided in Table 14 can be used to program a linear gradient on the HPLC analyzer.

TABLE 13
Oligonucleotide length and gradient percentages

| Length (bases) | Gradient (% B) |
|---|---|
| 0-5 | 0-30 |
| 6-10 | 10-40 |
| 11-16 | 20-50 |
| 17-32 | 30-60 |
| 33-50 | 40-70 |
| >50 | 50-80 |

TABLE 14
Parameters for a linear gradient on HPLC analyzer

| Time | Flow | % Buffer | % Buffer |
|---|---|---|---|
| (min) | (mL/min) | A | B |
| 0 | 1.5 | 100 | 0 |
| 1 | 1.5 | 100 | 0 |
| 3 | 1.5 | 70a | 30a |
| 15 | 1.5 | 40a | 60a |
| 15.5 | 2.5 | 0 | 100 |
| 17 | 2.5 | 0 | 100 |
| 17.25 | 2.5 | 100 | 0 |
| 23 | 2.5 | 100 | 0 |
| s23.1 | 1.5 | 100 | 0 |
| 24 | 1.5 | 100 | 0 |
| 25 | 0.1 | 100 | 0 |

### Example 4: Analysis of TREMs Via PAGE Purification and Analysis

This example describes the quality control of a synthesized TREM via PAGE Purification and Analysis. Gel purification and analysis of 2′-ACE protected RNA follows standard protocols for denaturing PAGE (Ellington and Pollard (1998) In Current Protocols in Molecular Biology, Chanda, V). Briefly, the 2′-ACE protected oligo is resuspended in 200 mL of gel loading buffer. Invitrogen™ NuPAGE™ 4-12% Bis-Tris Gels or similar gel is prepared in gel apparatus. Samples are loaded and gel ran at 50-120 W, maintaining the apparatus at 40° C. When complete, the gel is exposed to ultraviolet (UV) light at 254 nm to visualize the purity of the RNA using UV shadowing. If necessary, the desired gel band is excised with a clean razor blade. The gel slice is crushed and 0.3M NaOAc elution buffer is added to the gel particles, and soaked overnight. The mixture is decanted and filtered through a Sephadex column such as Nap-10 or Nap-25.

### Example 5: Deprotection of Synthesized TREM

This example describes the deprotection of a TREM made according to an in vitro synthesis method. The 2′-protecting groups are removed using 100 mM acetic acid, pH 3.8. The formic acid and ethylene glycol byproducts are removed by incubating at 60° C. for 30 min followed by lyophilization or SpeedVac-ing to dryness. After this final deprotection step, the oligonucleotides are ready for use.

### Example 6. Characterization of Chemically Modified TREMs for Readthrough of a Premature Termination Codon (PTC) in a Reporter Protein

This example describes an assay to test the ability of a chemically modified TREM to readthrough a PTC in a cell line expressing a reporter protein having a PTC. This protocol describes analysis of chemically modified TREM (i.e., Arg-TGA), though a non-cognate TREM specifying any one of the other amino acids can also be used.

A cell line engineered to stably express the NanoLuc reporter construct containing a premature termination codon (PTC) may be generated using the FlpIn and/or JumpIN cell system (ThermoFisher Scientific, USA) according to the manufacturer's instructions. The NanoLuc reporter can also be modified with a destabilizing PEST domain at its C-terminus to reduce the protein half-life. Delivery of the chemically modified TREMs into the NanoLuc reporter cells is carried out via a reverse transfection reaction using lipofectamine RNAiMAX (ThermoFisher Scientific, USA) according to manufacturer instructions. Briefly, 0.15 uL of a 5 uM solution of chemically modified TREM sample are diluted in a 7.35 uL RNAiMAX/OptiMEM mixture. After 30 min gentle mixing at room temperature, the 7.5 uL TREM/transfection mixture is added to a 384-well plate and kept still for 20-30 min before adding the cells stably expressing the NanoLuc reporter construct containing a PTC. The NanoLuc reporter cells are harvested and diluted to 4×10⁵ cells/mL in complete growth medium, and 30 μL of the diluted cell suspension is added and mixed to the plate containing the TREM.

To monitor the efficacy of the chemically modified TREM to read through the PTC in the reporter construct at 24 or 48 hours after TREM delivery into cells, a NanoGlo bioluminescent assay (Promega, USA) may be performed according to manufacturer instruction. Briefly, for cells that are to be assayed after 48 hours, 30 uL complete growth medium is added to the 384-well plate 24 hours post-transfection to dilute the transfection reagent for cell health. At the time of harvest, either 24-or 48-hours post-transfection, cell media is removed and plates are stored at −80 C. To perform the assay, thaw plates are thawed on ice for 1 hour, then transferred to room temperature and allowed to equilibrate. Add 15 uL room-temperature complete growth media to each well. NanoGlo reagent is prepared by mixing the buffer with substrate in a 50:1 ratio. 15 uL of mixed NanoGlo reagent is added to each well of the 384-well plate and mixed on the shaker at 600 rpm for 10 min. Then the plate is centrifuged at 10 0g for 1 min, followed by a 5 min incubation step at room temperature before measuring sample bioluminescence. As a positive control, a host cell expressing the NanoLuc reporter construct with a PTC is used and transfected with a previously-identified unmodified, active TREM. As a negative control, a host cell expressing the NanoLuc reporter construct with a PTC is used, but no TREM is transfected. The efficacy of the chemically modified TREMs is measured as a ratio of the NanoLuc luminescence in the experimental sample to the NanoLuc luminescence of the positive control or as a ratio of the NanoLuc luminescence in the experimental sample to the NanoLuc luminescence of the negative control. It is expected that if the sample TREM is functional, it may be able to read-through the stop mutation in the NanoLuc reporter and produce a luminescent reading higher than the luminescent reading measured in the negative control. If the sample TREM is not functional, the stop mutation is not rescued, and luminescence less or equal to the negative control is detected.

The impacts of chemical modifications were evaluated in singly and multiply modified TREM sequences and are summarized in FIG. 3. In this figure, the TREMs are annotated as follows: r: ribonucleotide; m: 2′-OMe; *: PS linkage; f: 2′-fluoro; moe: 2′-moe; d: deoxyribonucleotide; 5MeC: 5-methylcytosine. Thus, for example, mA represents 2′-O-methyl adenosine, moe5MeC represents 2′-MOE nucleotide with 5-methylcytosine nucleobase, and dA represents an adenosine deoxyribonucleotide.

FIG. 3 also summarizes the results of the activity screen in column “Activity A” for measurements made using NanoLuc reporter cells at 48 hours post-transfection, which reported as log 2 fold changes compared with the appropriate unmodified TREM, wherein “1” indicates less than a 1 log 2 fold change; “2” indicates greater than or equal to 1 and less than 3.32 log 2 fold change; and “3” indicates greater than or equal to 3.32 log 2 fold change. The results show that certain modifications were tolerated at many positions, but particular sites were sensitive to modification or exhibited improved activity when modified.

### Example 7: Characterization of Chemically Modified TREMs for Readthrough of a Premature Termination Codon (PTC) in a Disease Reporter Cell Line

This example describes an assay to test the ability of an exemplary TREM to readthrough a PTC in a cell line expressing a disease reporter protein bearing the PTC.

### Host Cell Modification

A cell line engineered to stably express a HaloTag and HiBiT-tagged disease reporter construct containing a premature termination codon (PTC), such as Factor IX at position 248 (FIX^{R218X}), Factor IX at position 29 (FIX^{R29X}), Factor IX at position 333 (FIX^{R333X}), or Rhodopsin at position 334 (Rho^{S334X}) was generated using the Jump-In system according to manufacturer's instructions. Briefly, Jump-In GripTite HEK293 (Thermo Scientific A14150) cells were co-transfected with an expression vector containing the disease reporter, such as pJTI-R4-DEST-CMV—FIX-R298X-HaloTag-HiBiT-pA for FIX^{R298X} to make the Factor IX disease reporter expressing cell line, and a pJTI-R₄-Int PhiC31 integrase expression vector using Lipofectamine2000 according to manufacturer's instructions. After 24 hours, the media was replaced with fresh media. The next day, the cells were re-seeded at 50% confluency and selected with 10 ug/mL Blasticidin and 600ug/mL G418 for 7 days with media change every 2 days. The remaining cells were expanded and tested for reporter construct expression.

### Translation Suppression Assay

Exemplary TREMs were synthesized and characterized as described herein, then transfected into cells. Forty-eight hours after TREM delivery into cells, conditioned media was collected, fresh media was added to the cells and allowed to equilibrate to room temperature. To measure the efficacy of arginine TREMs in PTC readthrough, full-length HiBiT-tagged disease reporter protein was assayed in both cells, and 48-hour conditioned media. Briefly, reconstituted Nano-Glo® HiBiT Lytic Reagent was added to both cells containing fresh media and 48-hour conditioned media at a 1:1 v/v ratio, mixed on an orbital shaker at 500 rpm for 10 minutes, and incubated at room temperature for 10 minutes. The HiBiT-tagged disease reporter activity is measured by reading the luminescence in a plate reader.

### Example 8: Pooled Screen for Increased Premature Termination Codon (PTC) Readthrough Activity of Exemplary TREMs Having Nucleotide Substitutions

This example describes a pooled screen of a plurality of exemplary TREMs bearing nucleotide substitutions to identify nucleotide substitutions that increase the premature termination codon (PTC) readthrough activity of the TREM.

### Pool Design

To perform a pooled screen of TREMs having nucleotide substitutions, a library of 5,000 to 100,000 unique TREM sequences were designed using saturating or sub-saturating mutagenesis strategies. The TREMs typically contain 1-10 nucleotide substitutions relative to one or more starting sequences. In this example, the library comprised TREMs corresponding to a parental Gln-TAG TREM sequence (e.g., SEQ ID NOs: 1654, 1660, or 1835)

### Pool Construction

The TREMs are synthesized as a single-stranded DNA oligo pool and then cloned using established techniques (e.g., as described in Yau et al., Methods Mol Biol, 2018) into a lentiviral vector that expresses the candidate tRNA from a PolIII promoter. The lentiviral pool is transduced into a reporter cell line where readthrough of a premature termination codon (PTC) in a reporter gene produces a fluorescent signal.

### Screen for Premature Termination Codon (PTC) Read-Through

Cells positive for fluorescence are isolated using fluorescence-activated cell sorting and the abundance of each TREM candidate in the positive population is compared to that of the input population using Next-Generation sequencing.

An enrichment score is calculated for each TREM as the relative abundance of each TREM in the positive population relative to the input population, with a higher enrichment score indicating that a TREM was positively associated with PTC readthrough. Each screen additionally contains 50 negative control sequences that are not expected to produce PTC readthrough and the enrichment scores are used to determine a hit threshold that excludes all or nearly all negative control sequences. Additionally, many human tRNAs that are converted to recognize the PTC are included and some of these are expected to score above the hit threshold. Novel TREMs above the hit threshold in two biological replicates are classified as hits. For example, a pooled screen for TREMs that recognize a TAG PTC and insert Gln was performed on 83,945 candidate tRNAs, with 801 candidates showing an enrichment score above the hit threshold of 2.5 in both replicates, e.g., as shown in FIG. 17.

### Hit Validation

A subset of hits were selected to be validated as individually synthesized RNA oligonucleotides. Hits for validation are selected based on two criteria: (i) They must span a range of enrichment scores above the hit threshold; (ii) They must differ from the parent sequence in different regions of the TREM two-dimensional structure. The diversity in enrichment scores and TREM sequences indicate which regions are amenable to mutations to produce an active PTC-suppressing TREM. The selected hits are synthesized as oligos and validated using a luciferase reporter assay. For example, 138 of the 801 hits from the Gln-TAG pooled screen shown in FIG. 17 were selected for validation as synthesized RNA oligonucleotides and evaluated in a luciferase reporter assay at two concentrations. As shown in FIG. 18, essentially all Gln-TAG candidates validated as having activity above background, and the majority were more active than the initial sequence from which candidates were derived.

Based on the screen and validation data, the hits identified during the Gln-TAG pooled screen represent a set of sequence changes to a starting TREM that typically lead to a new TREM sequence with maintained or greater PTC-suppression activity. The sequence changes that are overrepresented among hits in terms of nucleotide and structural changes across each position and TREM region are used to identify sequences changes that are likely to increase the activity of a starting Gln-TAG TREM when used individually or in combination. Each hit may provide a new sequence variant that can be used as the starting sequence for further rounds of pooled screening to improve its activity and identify additional nucleotide substitutions that may not have been among the dominant changes observed in a prior round of screening.

In addition to validating many Gln-TAG hits as synthesized oligonucleotides, 47 of these TREMs were additionally evaluated for their ability to promote PTC readthrough when delivered as lentiviral particles. The candidate TREMs selected here span a range of enrichment scores above the hit threshold, differ from the parent sequence in different regions of the TREM two-dimensional structure, and must have also been evaluated as synthesized oligonucleotides. For each selected TREM, a lentiviral transfer plasmid that will express the TREM from a PolIII promoter was produced using standard molecular cloning methods and then packaged to produce lentiviral particles. Cells that are transduced with the virus will express the TREM as well as a puromycin resistance gene. The titer of each virus was measured and then luciferase reporter cells with a TAG PTC mutation were transduced at a multiplicity of infection of 0.3 to create a cell line for each TREM candidate. Cells that were successfully transduced were selected using puromycin (2 ug/mL) over the next several days. At 7 days post-transduction 20,000 cells from each cell line were replated in a 96-well plate, and after growing a further 2 days cells were lysed and analyzed by luciferase assay to determine the PTC readthrough activity mediated by each TREM candidate. As shown in FIG. 22, the activity of the selected candidates delivered as synthesized oligonucleotides or lentiviral particles were well correlated, demonstrating that sequence engineering results from the pooled screens are suitable for RNA or viral delivery.

The results of pooled screens for each of various TREM scaffolds are summarized in FIGS. 23-25. FIG. 23 shows the oligo sequences of validated hits from the pooled screens and the log 2 PTC read-through activity of each TREM at low, intermediate, and high doses relative to the parent TREM, wherein “1” indicates no activity; “2” indicates active; and “3” indicates high activity. In this figure, the TREMs are annotated as follows: r: ribonucleotide; m: 2′-OMe. The results show that nucleotide substitutions at particular positions resulted in high PTC readthrough activity. FIG. 24 shows the nucleotide substitutions of validated hits listed in FIG. 23, the type of substitution, and the PTC read-through activity relative to the parent TREM at low and high doses, wherein “1” indicates less activity than the parent TREM, “2” equal activity as the parent TREM; and “3” indicates higher activity than the parent TREM. The results show that nucleotide substitutions at particular positions resulted in high PTC readthrough activity. FIG. 25 shows the PTC read-through activity of hits delivered as synthesized oligonucleotides as listed in FIGS. 23 and 24 in comparison with PTC read-through activity of the same hits delivered as lentiviral particles, wherein column Activity A lists the activity from lentiviral delivery and “1” indicates no activity; “2” indicates active; and “3” indicates high activity; column Activity B lists the activity from lentiviral delivery relative to the parent TREM and “1” indicates less activity than the parent TREM; “2” equal activity as the parent TREM; and “3” indicates higher activity than the parent TREM; column Activity C lists the activity from synthesized oligonucleotide delivery as shown in FIG. 23 and “1” indicates no activity; “2” indicates active; and “3” indicates high activity; and column Activity D lists the activity from synthesized oligonucleotide delivery relative to the parent TREM as shown in FIG. 24 and “1” indicates less activity than the parent TREM; “2” equal activity as the parent TREM; and “3” indicates higher activity than the parent TREM. The results show that the PTC read-through activity of hits is about equivalent when validated as synthesized oligonucleotides and by lentiviral delivery, e.g., the activity is not affected by the validation method of hits from the pooled screen.

What is claimed is:

1. A tRNA effector molecule (TREM) comprising a sequence of Formula (I): 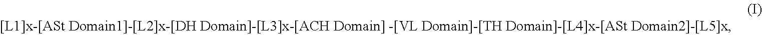
wherein:
independently, [L1] and [VL Domain], are optional;
x is 0 or 1; and
the TREM comprises a nucleotide substitution (e.g., a nucleotide mutation) in the TREM capable of modulating a functional parameter of the TREM.

2. The TREM of claim 1, wherein the functional parameter comprises activity of the TREM or stability of the TREM.

3. The TREM of any one of the preceding claims, wherein the functional parameter comprises premature termination codon (PTC) readthrough activity of the TREM (e.g., an increased level of PTC readthrough activity or decreased PTC readthrough activity), e.g., in a sample (e.g., a cell) or in a subject.

4. The TREM of any one of the preceding claims, wherein the functional parameter comprises stability of the TREM (e.g., increased stability of the TREM or decreased stability of the TREM), e.g., in a sample (e.g., a cell) or in a subject.

5. The TREM of any one of the preceding claims, wherein the functional parameter is selected from:
(a) protein translation;
(b) expression level (e.g., of polypeptide or protein, or mRNA);
(c) post-translational modification of polypeptide or protein;
(d) folding (e.g., of polypeptide or protein, or mRNA),
(e) structure (e.g., of polypeptide or protein, or mRNA),
(f) transduction (e.g., of polypeptide or protein),
(g) compartmentalization (e.g., of polypeptide or protein, or mRNA),
(h) incorporation (e.g., of polypeptide or protein, or mRNA) into a supermolecular structure, e.g., incorporation into a membrane, proteasome, or ribosome,
(i) incorporation into a multimeric polypeptide, e.g., a homo or heterodimer, and/or
(j) stability.

6. The TREM of claim 5, wherein the functional parameter further comprises:
(1) modulation of a signaling pathway, e.g., a cellular signaling pathway which is downstream or upstream of the protein encoded by the endogenous ORF having a first sequence or PTC;
(2) cell fate modulation;
(3) ribosome occupancy modulation;
(4) protein translation modulation;
(5) mRNA stability modulation;
(6) protein folding and structure modulation;
(7) protein transduction or compartmentalization modulation; and/or
(8) protein stability modulation.

7. The TREM of any one of the preceding claims, wherein the TREM comprises a TREM having at least 70%, 75%, 80%, 85%, 90%, 95%, or 99% sequence identity with a TREM provided in FIG. 3 or 23.

8. The TREM of any one of the preceding claims, wherein the TREM comprises a nucleotide sequence that differs by 2, 3, 4, 5, 6, 7, 8, 9, 10, 11, 12, 13, 14, 15, 16, 17, 18, 19, or nucleotides from the nucleotide sequence of a TREM provided in FIG. 3 or 23.

9. The TREM of any one of the preceding claims, wherein the TREM comprises a nucleotide sequence that differs by 10, 15, 20, 25, 30, 35 or 40 nucleotides from the nucleotide sequence of a TREM provided in FIG. 3 or 23.

10. The TREM of any one of the preceding claims, wherein the TREM comprises a nucleotide sequence that differs by more than 5 nucleotides from the nucleotide sequence of a TREM provided in FIG. 3 or 23.

11. The TREM of any one of the preceding claims, wherein the TREM comprises a nucleotide sequence that differs by more than 10 nucleotides from the nucleotide sequence of a TREM provided in FIG. 3 or 23.

12. The TREM of any one of the preceding claims, wherein the TREM comprises a sequence that differs by no more than 1, 2, 3, 4, 5, 6, 7, 8, 9, or 10 nucleotides from the nucleotide sequence of a TREM provided in FIG. 3 or 23.

13. The TREM of any one of the preceding claims, wherein the TREM comprises a nucleotide sequence that differs by no more than 10, 15, 20, 25, 30, 35 or 40 nucleotides from the nucleotide sequence of a TREM provided in FIG. 3 or 23.

14. The TREM of any one of the preceding claims, wherein the TREM comprises a nucleotide sequence that differs by no more than 1 nucleotide from the nucleotide sequence of a TREM provided in FIG. 3 or 23.

15. The TREM of any one of the preceding claims, wherein the TREM comprises a nucleotide sequence that differs by no more than 5 nucleotides from the nucleotide sequence of a TREM provided in FIG. 3 or 23.

16. The TREM of any one of the preceding claims, wherein the TREM comprises a nucleotide sequence that differs by no more than 10 nucleotides from the nucleotide sequence of a TREM provided in FIG. 3 or 23.

17. The TREM of any one of the preceding claims, wherein the TREM comprises a nucleotide substitution within the [ASt Domain1], e.g., the nucleotide substitution is at any of positions 1, 2, 3, 4, 5, 6, 7, 8, or 9 within the [ASt Domain1].

18. The TREM of any one of the preceding claims, wherein the TREM comprises a nucleotide substitution within the [DH Domain], e.g., the nucleotide substitution is at any of positions 10, 11, 12, 13, 14, 15, 16, 17, 18, 19, 20, 21, 22, 23, 24, 25, or 26 within the [DH Domain].

19. The TREM of any one of the preceding claims, wherein the TREM comprises a nucleotide substitution within the [ACH Domain], e.g., the nucleotide substitution is at any of positions 27, 28, 29, 30, 31, 32, 33, 34, 35, 36, 37, 38, 39, 40, 41, 42, or 43 within the [ACH Domain].

20. The TREM of any one of the preceding claims, wherein the TREM comprises a nucleotide substitution within the [VL Domain], e.g., the nucleotide substitution is at any of positions 44, 45, 46, 47, 48, or 49 within the [VL Domain].

21. The TREM of any one of the preceding claims, wherein the TREM comprises a nucleotide substitution within the [TH Domain], e.g., the nucleotide substitution is at any of positions 50, 51, 52, 53, 54, 55, 56, 57, 58, 59, 60, 61, 62, 63, or 64 within the [TH Domain].

22. The TREM of any one of the preceding claims, wherein the TREM comprises a nucleotide substitution within the [ASt Domain2], e.g., the nucleotide substitution is at any of positions 65, 66, 67, 68, 69, 70, 71, 72, 73, 74, 75, or 76 within the [ASt Domain2].

23. The TREM of any one of the preceding claims, wherein the TREM has a sequence selected from a sequence provided in FIG. 3 or 23.

24. The TREM of any one of the preceding claims, wherein the TREM comprises a plurality of nucleotide substitutions.

25. The TREM of any one of any one of the preceding claims, wherein the nucleotide substitution comprises substituting a first naturally occurring nucleotide with an adenosine, guanosine, cytosine, or uracil nucleotide.

26. The TREM of any one of the preceding claims, wherein the TREM is selected from SEQ ID NOs. 625-700, 701-800, 801-900, 901-1000, 1001-1100, 1101-1151, 1290-1300, 1301-1400, or 1401-1456 in FIG. 3.

27. The TREM of any one of the preceding claims, wherein the TREM comprising a nucleotide substitution does not exhibit a difference in a functional parameter from a TREM in FIG. 3 or 23.

28. The TREM of any one of claims 1-26, wherein the TREM comprising a nucleotide substitution exhibits a difference in a functional parameter from a TREM in FIG. 3 or 23.

29. The TREM of any one of the preceding claims, wherein the TREM comprising a nucleotide substitution exhibits an improvement in a functional parameter from a TREM in FIG. 3 or 23.

30. The TREM of any one of the preceding claims, wherein the improvement comprises an improvement of about 5%, 10%, 15%, 20%, 25%, 30% 35%, 40%, 45%, 50%, 55%, 60%, 65%, 70%, 75%, 80%, 85%, 90%, 95%, or greater, e.g., compared with the functional parameter of a TREM provided in FIG. 3 or 23.

31. The TREM of any one of the preceding claims, wherein the TREM comprising a nucleotide substitution exhibits a reduction in a functional parameter from a TREM in FIG. 3 or 23.

32. The TREM of any one of the preceding claims, wherein the reduction comprises a reduction of about 5%, 10%, 15%, 20%, 25%, 30%, 35%, 40%,45%, 50%, 55%, 60%, 65%, 70%, 75%, 80%, 85%, 90%, 95%, or greater, e.g., compared with the functional parameter of a TREM provided in FIG. 3 or 23.

33. The TREM of any one of the preceding claims, wherein the TREM comprises a nucleotide substitution at any of positions 1, 2, 3, 4, 5, 6, or 7, e.g., within the [ASt Domain1].

34. The TREM of any one of the preceding claims, wherein the TREM comprises a nucleotide substitution at any of positions 16, 17, or 20a, e.g., within the [DH Domain].

35. The TREM of any one of the preceding claims, wherein the TREM comprises a nucleotide substitution at any of positions 27, 28, 29, 30, 31, 39, 40, 41, 42, or 43, e.g., within the [ACH Domain].

36. The TREM of any one of the preceding claims, wherein the TREM comprises a nucleotide substitution at any of positions 51, 59, 60, or 63, e.g., within the [TH Domain].

37. The TREM of any one of the preceding claims, wherein the TREM comprises a nucleotide substitution at any of positions 66, 67, 68, 69, 70, 71, 72, or 73, e.g., within the [ASt Domain2].

38. The TREM of any one of the preceding claims, wherein the TREM comprises a nucleotide substitution at any of positions 32, 33, 34, 35, 36, 37, or 38, e.g., within the [ACH Domain].

39. The TREM of any one of the preceding claims, wherein the TREM comprising a nucleotide substitution at any of positions 1, 2, 3, 4, 5, 6, or 7, e.g., within the [ASt Domain1], exhibits an improvement in a functional parameter of the TREM.

40. The TREM of any one of the preceding claims, wherein the TREM comprising a nucleotide substitution at any of positions 16, 17, or 20a, e.g., within the [DH Domain], exhibits an improvement in a functional parameter of the TREM.

41. The TREM of any one of the preceding claims, wherein the TREM comprising a nucleotide substitution at any of positions 27, 28, 29, 30, 31, 39, 40, 41, 42, or 43, e.g., within the [ACH Domain], exhibits an improvement in a functional parameter of the TREM.

42. The TREM of any one of the preceding claims, wherein the TREM comprising a nucleotide substitution at any of positions 51, 59, 60, or 63, e.g., within the [TH Domain], exhibits an improvement in a functional parameter of the TREM.

43. The TREM of any one of the preceding claims, wherein the TREM comprising a nucleotide substitution at any of positions 66, 67, 68, 69, 70, 71, 72, or 73, e.g., within the [ASt Domain2], exhibits an improvement in a functional parameter of the TREM.

44. The TREM of any one of the preceding claims, wherein the TREM comprising a nucleotide substitution at any of positions 32, 33, 34, 35, 36, 37, or 38, e.g., within the [ACH Domain], exhibits a reduction in a functional parameter of the TREM.

45. The TREM of any one of the preceding claims, wherein the TREM comprising a nucleotide substitution comprises a scaffold, e.g., an Arg-TGA, Gln-TAG, Glu-TAG, Leu-TAG, Tyr-TAG, Ser-TAG, or Lys-TAG scaffold, e.g., a TREM corresponding to a reference sequence of SEQ ID NO: 1638, 1835, 1660, 1654, 1867, 2000, 2001, 2016, 2017, 2022, 2023, 2020, 2021, 2018, or 2019.

46. The TREM of any one of the preceding claims, wherein the TREM comprising a nucleotide substitution corresponds to a reference sequence of SEQ ID NO: 1638, 1835, 1660, 1654, 1867, 2000, 2001, 2016, 2017, 2022, 2023, 2020, 2021, 2018, or 2019.

47. The TREM of any one of the preceding claims, wherein the TREM comprises a non-naturally occurring modification.

48. The TREM of claim 47, wherein the non-naturally occurring modification is present on the 2′-position of a nucleotide sugar or within the internucleotide region (e.g., a backbone modification).

49. The TREM of any one of claims 47-48, wherein the non-naturally occurring modification is selected from a 2′-O-methyl (2-OMe), 2′-halo (e.g., 2′F or 2′C1), 2′-O-methoxyethyl (2′MOE), or 2′deoxy modification.

50. The TREM of any one of claims 47-49, wherein the non-naturally occurring modification is a phosphorothioate modification.

51. The TREM of any one of claims 47-50, wherein the TREM comprises 1, 2, 3, 4, 5, 6, 7, 8, 9, or 10 additional non-naturally occurring modifications compared with a TREM provided in FIG. 3 (e.g., 2′-ribose modifications or an internucleotide modification, e.g., 2′OMe, 2′-halo, 2′-MOE, 2′-deoxy, or phosphorothiorate modifications).

52. The TREM of any one of claims 47-51, wherein the TREM comprises a pattern of non-naturally occurring modifications selected from the patterns listed in Table 6.

53. The TREM of any one of the preceding claims, wherein:
(i) the non-naturally occurring modification is present at a nucleotide position which corresponds to one or more of nucleotides 1-9 of SEQ ID NO: 734; and/or
(ii) the TREM differs from the nucleotide sequence of SEQ ID NO: 734 by about 1, 2, 3, 4, 5, 6, 7, 8, 9, or 10 nucleotides.

54. The TREM of any one of the preceding claims, wherein:
(i) the non-naturally occurring modification is present at a nucleotide position which corresponds to one or more of nucleotides 10-19 of SEQ ID NO: 734; and/or
(ii) the TREM differs from the nucleotide sequence of SEQ ID NO: 734 by about 1, 2, 3, 4, 5, 6, 7, 8, 9, or 10 nucleotides.

55. The TREM of any one of the preceding claims, wherein:
(i) the non-naturally occurring modification is present at a nucleotide position which corresponds to one or more of nucleotides 20-29 of SEQ ID NO: 734; and/or
(ii) the TREM differs from the nucleotide sequence of SEQ ID NO: 734 by about 1, 2, 3, 4, 5, 6, 7, 8, 9, or 10 nucleotides.

56. The TREM of any one of the preceding claims, wherein:
(i) the non-naturally occurring modification is present at a nucleotide position which corresponds to one or more of nucleotides 30-39 of SEQ ID NO: 734; and/or
(ii) the TREM differs from the nucleotide sequence of SEQ ID NO: 734 by about 1, 2, 3, 4, 5, 6, 7, 8, 9, or 10 nucleotides.

57. The TREM of any one of the preceding claims, wherein:
(i) the non-naturally occurring modification is present at a nucleotide position which corresponds to one or more of nucleotides 40-49 of SEQ ID NO: 734; and/or
(ii) the TREM differs from the nucleotide sequence of SEQ ID NO: 734 by about 1, 2, 3, 4, 5, 6, 7, 8, 9, or 10 nucleotides.

58. The TREM of any one of the preceding claims, wherein:
(i) the non-naturally occurring modification is present at a nucleotide position which corresponds to one or more of nucleotides 50-59 of SEQ ID NO: 734; and/or
(ii) the TREM differs from the nucleotide sequence of SEQ ID NO: 734 by about 1, 2, 3, 4, 5, 6, 7, 8, 9, or 10 nucleotides.

59. The TREM of any one of the preceding claims, wherein:
(i) the non-naturally occurring modification is present at a nucleotide position which corresponds to one or more of nucleotides 60-69 of SEQ ID NO: 734; and/or
(ii) the TREM differs from the nucleotide sequence of SEQ ID NO: 734 by about 1, 2, 3, 4, 5, 6, 7, 8, 9, or 10 nucleotides.

60. The TREM of any one of the preceding claims, wherein:
(i) the non-naturally occurring modification is present at a nucleotide position which corresponds to one or more of nucleotides 70-76 of SEQ ID NO: 734; and/or
(ii) the TREM differs from the nucleotide sequence of SEQ ID NO: 734 by about 1, 2, 3, 4, 5, 6, 7, 8, 9, or 10 nucleotides.

61. The TREM of any one of the preceding claims, wherein the non-naturally occurring modification is present at a nucleotide position which corresponds to one or more of nucleotides 1-76, according to the CtNS.

62. A pharmaceutical composition comprising a TREM of any one of the preceding claims.

63. The pharmaceutical composition of claim 90, further comprising a pharmaceutically acceptable component, e.g., an excipient.

64. A lipid nanoparticle formulation comprising a TREM of any one of claims 1-91.

65. A lipid nanoparticle formulation comprising a pharmaceutical composition of claim 92.

66. A composition for use in treating a subject having a disease or disorder associated with a PTC comprising administering to the subject a TREM described herein (e.g., a TREM of any one of the preceding claims).

67. The composition for use of claim 66, wherein the disease or disorder associated with a PTC comprises Hemophilia B, Fabry disease, Usher syndrome, or CLN2 disease.

68. A method of treating a subject having a disease or disorder associated with a PTC comprising administering to the subject a TREM described herein (e.g., a TREM of any one of the preceding claims), thereby treating the subject having the disease or disorder.

69. The method of claim 68, wherein the disease or disorder associated with a PTC comprises Hemophilia B, Fabry disease, Usher syndrome, or CLN2 disease.

70. A method of modulating a functional parameter of a tRNA effector molecule (TREM), wherein the TREM comprises a sequence of Formula (A): 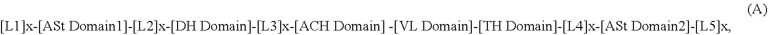
wherein:
independently, [L1] and [VL Domain], are optional;
x is 0 or 1; and
the TREM comprises a nucleotide substitution (e.g., a nucleotide mutation) in the TREM capable of modulating a functional parameter of the TREM;
thereby modulating the functional parameter of the TREM.
